(19) United States
(12) Patent Application Publication
(10) Pub. No.: US 2019/0290692 A1
(43) Pub. Date: Sep. 26, 2019

(54) HUMANIZED ANTI-MUC1* ANTIBODIES AND USE OF CLEAVAGE ENZYME

(71) Applicant: MINERVA BIOTECHNOLOGIES CORPORATION, Waltham, MA (US)

(72) Inventors: Cynthia Bamdad, Waltham, MA (US); Benoit Smagghe, Waltham, MA (US); Luke Deary, Waltham, MA (US)

(73) Assignee: MINERVA BIOTECHNOLOGIES CORPORATION, Waltham, MA (US)

(21) Appl. No.: 16/340,974

(22) PCT Filed: Oct. 11, 2017
(86) PCT No.: PCT/US17/56204

§ 371 Date: Apr. 10, 2019

Related U.S. Application Data
(60) Provisional application No. 62/555,950, filed on Sep. 8, 2017.
(60) Provisional application No. 62/487,999, filed on Apr. 20, 2017.
(60) Provisional application No. 62/462,667, filed on Feb. 23, 2017.
(60) Provisional application No. 62/461,170, filed on Feb. 20, 2017.
(60) Provisional application No. 62/406,878, filed on Oct. 11, 2016.

(51) Int. Cl. A61K 35/17; A61P 35/00; C12N 15/85; C07K 16/30; C07K 14/725; C12N 5/0783

(52) U.S. Cl. CPC A61K 35/17; A61P 35/00; C12N 15/85; C07K 16/3092; C07K 14/7051; C07K 2317/24; C07K 2317/622; C07K 2317/73; C07K 2319/03; C07K 2319/033; C12N 5/0638

(57) ABSTRACT

The present application discloses humanized antibodies and antibody like proteins and fragments thereof and use of cleavage enzyme.

### BACKGROUND OF THE INVENTION

### 1. Field of the Invention

The present application relates to humanized and non-human anti-MUC1* antibodies and methods of making and using them. The present application also relates to using an immune cell transfected or transduced with a cleavage enzyme for the treatment of cancer. The present invention also relates to using an immune cells transfected or transduced with a CAR and another protein for the treatment of cancer.

### 2. General Background and State of the Art

We previously discovered that a cleaved form of the MUC1 (SEQ ID NO:1) transmembrane protein is a growth factor receptor that drives the growth of over 75% of all human cancers. The cleaved form of MUC1, which we called MUC1* (pronounced muk 1 star), is a powerful growth factor receptor. Cleavage and release of the bulk of the extracellular domain of MUC1 unmasks a binding site for activating ligands dimeric NME1, NME6, NME7, NME7_{AB}, NME7-X1 or NME8. It is an ideal target for cancer drugs as it is aberrantly expressed on over 75% of all cancers and is likely overexpressed on an even higher percentage of metastatic cancers (Mahanta et al. (2008) A Minimal Fragment of MUC1 Mediates Growth of Cancer Cells. PLoS ONE 3(4): e2054. doi:10.1371/journal.pone.0002054; Fessler et al. (2009), “MUC1* is a determinant of trastuzumab (Herceptin) resistance in breast cancer cells,” Breast Cancer Res Treat. 118(1):113-124). After MUC1 cleavage most of its extracellular domain is shed from the cell surface. The remaining portion has a truncated extracellular domain that comprises most or all of the primary growth factor receptor sequence called PSMGFR (SEQ ID NO:2).

Antibodies are increasingly used to treat human diseases. Antibodies generated in non-human species have historically been used as therapeutics in humans, such as horse antibodies. More recently, antibodies are engineered or selected so that they contain mostly human sequences in order to avoid a generalized rejection of the foreign antibody. The process of engineering recognition fragments of a non-human antibody into a human antibody is generally called ‘humanizing’. The amount of non-human sequences that are used to replace the human antibody sequences determines whether they are called chimeric, humanized or fully human.

Alternative technologies exist that enable generation of humanized or fully human antibodies. These strategies involve screening libraries of human antibodies or antibody fragments and identifying those that bind to the target antigen, rather than immunizing an animal with the antigen. Another approach is to engineer the variable region(s) of an antibody into an antibody-like molecule. Another approach involves immunizing a humanized animal. The present invention is intended to also encompass these approaches for use with recognition fragments of antibodies that the inventors have determined bind to the extracellular domain of MUC1*.

In addition to treating patients with an antibody, cancer immunotherapies have recently been shown to be effective in the treatment of blood cancers. One cancer immunotherapy, called CAR T (chimeric antigen receptor T cell) therapy, engineers a T cell so that it expresses a chimeric receptor having an extra cellular domain that recognizes a tumor antigen, and a transmembrane and cytoplasmic tail of a T cell (Dai H, Wang Y, Lu X, Han W. (2016) Chimeric Antigen Receptors Modified T-Cells for Cancer Therapy. J Natl Cancer Inst. 108(7): djv439). Such receptor is composed of an single chain antibody fragment (scFv) that recognizes a tumor antigen, linked to a T cell transmembrane and signaling domains. Upon binding of the receptor to a cancer associated antigen, a signal is transmitted resulting in T-cell activation, propagation and the targeted killing of the cancer cells. In practice, a patient's T cells are isolated and transduced with a CAR, expanded and then injected back into the patient. When the patient's CAR T cells bind to the antigen on a cancer cell, the CAR T cells expand and attack the cancer cells. A drawback of this method is the risk of activating the patient's immune system to destroy cells bearing the target antigen, when most cancer antigens are expressed on some healthy tissues, but overexpressed on cancerous tissues. To minimize the risk of off-tumor/on-target effects, the cancer antigen should be minimally expressed on healthy tissues.

Another cancer immunotherapy involves BiTEs (Bi-specific T cell Engagers). The BiTE approach attempts to eliminate the CAR T associated risk of off-tumor/on-target effects. Unlike CAR T, BiTEs are bispecific antibodies that should not pose any greater risk than regular antibody-based therapies. However, unlike typical anti-cancer antibodies that bind to and block a cancer antigen, BiTEs are designed to bind to an antigen on the tumor cell and simultaneously bind to an antigen on an immune cell, such as a T cell. In this way, a BiTE recruits the T cell to the tumor. BiTEs are engineered proteins that simultaneously bind to a cancer associated antigen and a T-cell surface protein such as CD3-epsilon. BiTEs are antibodies made by genetically linking the scFv's of an antibody that binds to a T cell antigen, like anti-CD3-epsilon to a scFv of a therapeutic monoclonal antibody that binds to a cancer antigen (Patrick A. Baeuerle, and Carsten Reinhardt (2009) Bispecific T-cell engaging antibodies for cancer therapy. Cancer Res. 69(12):4941-4944).

### SUMMARY OF THE INVENTION

In one aspect, the present invention is directed to a non-human, human or humanized anti-MUC1* antibody or antibody fragment or antibody-like protein that binds to a region on extracellular domain of MUC1 isoform or cleavage product that is devoid of the tandem repeat domains. The non-human, human or humanized anti-MUC1* antibody or antibody fragment or antibody-like protein may specifically bind to

(i) PSMGFR region of MUC1;

(ii) PSMGFR peptide;

(iii) a peptide having amino acid sequence of SNIKFRPGSVVVQLTLAFREGTINVHDVETQFNQYKTEAASRY (SEQ ID NO:620);

(iv) a peptide having amino acid sequence of SVVVQLTLAFREGTINVHDVETQFNQYKTEAASRY (SEQ ID NO:621);

(v) a peptide having amino acid sequence of VQLTLAFREGTINVHDVETQFNQY (SEQ ID NO:622); or

(vi) a peptide having amino acid sequence of SNIKFRPGSVVVQLTLAFREGTIN (SEQ ID NO:623).

The non-human, human or humanized antibody may be IgG1, IgG2, IgG3, IgG4 or IgM. The human or humanized antibody fragment or antibody-like protein may be scFv or scFv-Fc.

The human or humanized antibody, antibody fragment or antibody-like protein as in above may comprise a heavy chain variable region and light chain variable region which is derived from mouse monoclonal MN-E6 antibody, and has at least 80%, 90% or 95% or 98% sequence identity to the mouse monoclonal MN-E6 antibody. The heavy chain variable region may have at least 90% or 95% or 98% sequence identity to SEQ ID NO:13 and the light chain variable region may have at least 90% or 95% or 98% sequence identity to SEQ ID NO:66.

The human or humanized antibody, antibody fragment or antibody-like protein according to above may include complementarity determining regions (CDRs) in the heavy chain variable region and light chain variable region having at least 90% or 95% or 98% sequence identity to CDR1, CDR2 or CDR3 regions having sequence as follows:

CDR1 heavy chain SEQ ID NO:17

CDR1 light chain SEQ ID NO:70,

CDR2 heavy chain SEQ ID NO:21

CDR2 light chain SEQ ID NO:74,

CDR3 heavy chain SEQ ID NO:25

CDR3 light chain SEQ ID NO:78.

The human or humanized antibody, antibody fragment or antibody-like protein described above may include a heavy chain variable region and light chain variable region which is derived from mouse monoclonal MN-C2 antibody, and has at least 80%, 90% or 95% or 98% sequence identity to the mouse monoclonal MN-C2 antibody. The heavy chain variable region may have at least 90% or 95% or 98% sequence identity to SEQ ID NO:119 and the light chain variable region has at least 90% or 95% or 98% sequence identity to SEQ ID NO:169. The complementarity determining regions (CDRs) in the heavy chain variable region and light chain variable region may have at least 90% or 95% or 98% sequence identity to CDR1, CDR2 or CDR3 regions having sequence as follows:

CDR1 heavy chain SEQ ID NO:123

CDR1 light chain SEQ ID NO: 173,

CDR2 heavy chain SEQ ID NO: 127

CDR2 light chain SEQ ID NO: 177,

CDR3 heavy chain SEQ ID NO:131

CDR3 light chain SEQ ID NO:181.

The human or humanized antibody, antibody fragment or antibody-like protein as in above may include a heavy chain variable region and light chain variable region which is derived from mouse monoclonal MN-C3 antibody, and may have at least 80%, 90% or 95% or 98% sequence identity to the mouse monoclonal MN-C3 antibody. The heavy chain variable region may have at least 90% or 95% or 98% sequence identity to SEQ ID NO:414 and the light chain variable region may have at least 90% or 95% or 98% sequence identity to SEQ ID NO:459. The complementarity determining regions (CDRs) in the heavy chain variable region and light chain variable region may have at least 90% or 95% or 98% sequence identity to CDR1, CDR2 or CDR3 regions having sequence as follows:

CDR1 heavy chain SEQ ID NO:418

CDR1 light chain SEQ ID NO:463,

CDR2 heavy chain SEQ ID NO:422

CDR2 light chain SEQ ID NO:467,

CDR3 heavy chain SEQ ID NO:426,

CDR3 light chain SEQ ID NO:471.

The human or humanized antibody, antibody fragment or antibody-like protein described above may include a heavy chain variable region and light chain variable region which is derived from mouse monoclonal MN-C8 antibody, and has at least 80%, 90% or 95% or 98% sequence identity to the mouse monoclonal MN-C8 antibody. The heavy chain variable region may have at least 90% or 95% or 98% sequence identity to SEQ ID NO:506 and the light chain variable region may have at least 90% or 95% or 98% sequence identity to SEQ ID NO:544. The complementarity determining regions (CDRs) in the heavy chain variable region and light chain variable region may have at least 90% or 95% or 98% sequence identity to CDR1, CDR2 or CDR3 regions having sequence as follows:

CDR1 heavy chain SEQ ID NO:508

CDR1 light chain SEQ ID NO:546,

CDR2 heavy chain SEQ ID NO:510

CDR2 light chain SEQ ID NO:548,

CDR3 heavy chain SEQ ID NO:512,

CDR3 light chain SEQ ID NO:550.

In another aspect, the present invention is directed to an anti-MUC1* extracellular domain antibody comprised of sequences of a humanized MN-E6 represented by humanized IgG2 heavy chain, or humanized IgG1 heavy chain, paired with humanized Kappa light chain, or humanized Lambda light chain. The humanized IgG2 heavy chain may be SEQ ID NOS:53, humanized IgG1 heavy chain may be SEQ ID NO:57, humanized Kappa light chain may be SEQ ID NO: 108, and humanized Lambda light chain may be SEQ ID NO: 112, or a sequence having 90%, 95% or 98% sequence identity thereof.

In another aspect, the invention is directed to an anti-MUC1* extracellular domain antibody comprised of sequences of a humanized MN-C2 represented by humanized IgG1 heavy chain, humanized IgG2 heavy chain, paired with humanized Lambda light chain, and humanized Kappa light chain. The humanized IgG1 heavy chain MN-C2 may be SEQ ID NOS:159 or IgG2 heavy chain may be SEQ ID NOS:164 paired with Lambda light chain (SEQ ID NO:219) or Kappa light chain (SEQ ID NO:213), or a sequence having 90%, 95% or 98% sequence identity thereof.

In another aspect, the invention is directed to an anti-MUC1* extracellular domain antibody comprised of sequences of a humanized MN-C3 represented by humanized IgG1 heavy chain or humanized IgG2 heavy chain paired with humanized Lambda light chain or humanized Kappa light chain. The humanized MN-C3 IgG1 heavy chain may be SEQ ID NOS:454, IgG2 heavy chain may be SEQ ID NOS:456, Lambda light chain may be SEQ ID NO:501, and Kappa light chain may be SEQ ID NO:503, or a sequence having 90%, 95% or 98% sequence identity thereof.

In another aspect, the invention is directed to an anti-MUC1* extracellular domain antibody comprised of sequences of a humanized MN-C8 represented by humanized IgG1 heavy chain or humanized IgG2 heavy chain paired with humanized Lambda light chain or humanized Kappa light chain. The humanized MN-C8 IgG1 heavy chain may be SEQ ID NOS:540, IgG2 heavy chain may be SEQ ID NOS:542, Lambda light chain may be SEQ ID NO:580 and Kappa light chain may be SEQ ID NO:582, or a sequence having 90%, 95% or 98% sequence identity thereof.

In another aspect, the invention is directed to a human or humanized anti-MUC1* antibody or antibody fragment or antibody-like protein according to above, which inhibits the binding of NME protein to MUC1*. The NME may be NME1, NME6, NME7AB, NME7-X1, NME7 or NME8.

In yet another aspect, the invention is directed to a single chain variable fragment (scFv) comprising a heavy and light chain variable regions connected via a linker, further comprising CDRs of antibodies that bind to MUC1* extracellular domain. The CDRs may be derived from MN-E6, MN-C2, MN-C3 or MN-C8 antibodies or humanized antibodies thereof. The scFv may be one that possesses the SEQ ID NOS:233, 235 and 237 (E6); SEQ ID NOS:239, 241, and 243 (C2); SEQ ID NOS:245, 247, and 249 (C3); or SEQ ID NOS:251, 253, and 255 (C8).

In still another aspect, the invention is directed to a chimeric antigen receptor (CAR) comprising a scFv or a humanized variable region that binds to the extracellular domain of a MUC1 that is devoid of tandem repeats, a linker molecule, a transmembrane domain and a cytoplasmic domain. The single chain antibody fragment may bind to

(i) PSMGFR region of MUC1,

(ii) PSMGFR peptide,

(iii) a peptide having amino acid sequence SNIKFRPGSVVVQLTLAFREGTINVHDVETQFNQYKTEAASRY (SEQ ID NO:620);

(iv) a peptide having amino acid sequence of SVVVQLTLAFREGTINVHDVETQFNQYKTEAASRY (SEQ ID NO:621);

(v) a peptide having amino acid sequence of VQLTLAFREGTINVHDVETQFNQY (SEQ ID NO:622); or

(vi) a peptide having amino acid sequence of SNIKFRPGSVVVQLTLAFREGTIN (SEQ ID NO:623).

In the CAR as described above, portions of any of the variable regions set forth and described above, or combination thereof may be used in the extracellular domain of the CAR. The CAR also comprises a transmembrane region and a cytoplasmic tail that comprises sequence motifs that signal immune system activation. The extracellular domain may be comprised of non-human, or humanized single chain antibody fragments of an MN-E6 scFv, MN-C2 scFv, MN-C3 scFv or MN-C8 scFv.

In the CAR as described above, the extracellular domain may include a non-human or humanized single chain antibody fragments of an MN-E6 scFv set forth as SEQ ID NOS: 233, 235, or 237), MN-C2 scFv (SEQ ID NOS:239, 241, or 243), MN-C3 scFv (SEQ ID NOS: 245, 247, or 249) or MN-C8 scFv (SEQ ID NOS:251, 253, or 255).

In any of the CARs described above, the cytoplasmic tail may be comprised of one or more of signaling sequence motifs CD3-zeta, CD27, CD28, 4-1BB, OX40, CD30, CD40, ICAm-1, LFA-1, ICOS, CD2, CD5, or CD7.

In any of the CARs described above, the sequence may be CARMN-E6 CD3z (SEQ ID NOS:295), CARMN-E6 CD28/CD3z (SEQ ID NOS:298); CARMN-E6 4-1BB/CD3z (SEQ ID NOS:301); CARMN-E6 OX40/CD3z (SEQ ID NOS:617); CARMN-E6 CD28/4-1BB/CD3z (SEQ ID NOS:304); CARMN-E6 CD28/OX40/CD3z (SEQ ID NOS:619); CAR MN-C2 CD3z (SEQ ID NOS:607); CAR MN-C2 CD28/CD3z (SEQ ID NOS:609); CAR MN-C2 4-1BB/CD3z (SEQ ID NOS:611 and SEQ ID NOS: 719); CAR MN-C2 OX40/CD3z (SEQ ID NOS:613); CAR MN-C2 CD28/4-1BB/CD3z (SEQ ID NOS: 307); CAR MN-C2 CD28/OX40/CD3z (SEQ ID NOS:615) or CAR MN-C3 4-1BB/CD3z (SEQ ID NOS: 601).

In another aspect, the CAR may have an extracellular domain unit that recognizes a peptide. The peptide may be PSMGFR (SEQ ID NO:2). The peptide may be a peptide derived from NME7. The peptide may be

NME7A peptide 1 (A domain): MLSRKEALDFHVDHQS (SEQ ID NO:7);

NME7A peptide 2 (A domain): SGVARTDASES (SEQ ID NO:8);

NME7B peptide 1 (B domain): DAGFEISAMQMFNMDRVNVE (SEQ ID NO:9);

NME7B peptide 2 (B domain): EVYKGVVTEYHDMVTE (SEQ ID NO:10); or

NME7B peptide 3 (B domain): AIFGKTKIQNAVHCTDLPEDGLLEVQYFF (SEQ ID NO:11).

In another aspect, the invention is directed to a composition that includes at least two CARs with different extracellular domain units transfected into the same cell.

The at least two CARs may have one CAR that does not have a tumor antigen targeting recognition unit and the other CAR does have a tumor antigen targeting recognition unit. In another aspect of the invention, one of the extracellular domain recognition units may bind to MUC1* extracellular domain. In another aspect of the invention, one of the extracellular domain recognition units may be an antibody fragment and the other is a peptide, which may be devoid of transmembrane and signaling motifs; the peptide may be a single chain antibody fragment. In another aspect of the invention, one of the recognition units may bind PD-1 or PDL-1. In another aspect of the invention, one extra cellular domain recognition unit is an anti-MUC1* scFv chosen from the group consisting of scFv of MN-E6 antibody, scFv of MN-C2 antibody, scFv of MN-C3 antibody or scFv of MN-C8 antibody and the other is a peptide derived from NME7 or chosen from the group consisting of

NME7A peptide 1 (A domain): MLSRKEALDFHVDHQS (SEQ ID NO:7);

NME7A peptide 2 (A domain): SGVARTDASES (SEQ ID NO:8);

NME7B peptide 1 (B domain): DAGFEISAMQMFNMDRVNVE (SEQ ID NO:9);

NME7B peptide 2 (B domain): EVYKGVVTEYHDMVTE (SEQ ID NO:10); and

NME7B peptide 3 (B domain): AIFGKTKIQNAVHCTDLPEDGLLEVQYFF (SEQ ID NO:11).

In another aspect, the invention is directed to a cell comprising a CAR with an extracellular domain that binds to the extra cellular domain of a MUC1 molecule that is devoid of tandem repeats. In another aspect, the invention is directed to a cell comprising a CAR with an extracellular domain that binds to a MUC1* transfected or transduced cell. The cell that includes the CAR may be an immune system cell, preferably a T cell, a natural killer cell (NK), a dendritic cell or mast cell.

In another aspect, the invention is directed to an engineered antibody-like protein.

In another aspect, the invention is directed to a method of screening a library of antibodies or antibody fragments that are human, for those that bind to

(i) PSMGFR peptide;

(ii) a peptide having amino acid sequence SNIKFRPGSVVVQLTLAFREGTINVHDVETQFNQYKTEAASRY (SEQ ID NO:620);

(iii) a peptide having amino acid sequence of SVVVQLTLAFREGTINVHDVETQFNQYKTEAASRY (SEQ ID NO:621);

(iv) a peptide having amino acid sequence of VQLTLAFREGTINVHDVETQFNQY (SEQ ID NO:622);

(v) a peptide having amino acid sequence of SNIKFRPGSVVVQLTLAFREGTIN (SEQ ID NO:623);

(vi) NME7 protein; or

(vii) a peptide fragment of NME7 protein.

In another aspect, the invention is directed to a method for treating a disease in a subject comprising administering an antibody according to any claim above, to a person suffering from the disease, wherein the subject expresses MUC1 aberrantly. The disease may be cancer, such as breast cancer, ovarian cancer, lung cancer, colon cancer, gastric cancer or esophageal cancer.

In another aspect, the invention is directed to a method for treating a disease in a subject comprising administering an NME peptide, to a person suffering from the disease, wherein the subject expresses MUC1 aberrantly.

In another aspect, the invention is directed to a method of proliferating or expanding stem cell population comprising contacting the cells with the antibody according to any method or composition described above.

In another aspect, the invention is directed to a method of facilitating stem cell attachment to a surface comprising coating the surface with a humanized MN-C3 or MN-C8 antibody, antibody fragment or single chain antibody thereof and contacting stem cell to the surface.

In another aspect, the invention is directed to a method of delivering stem cell in vitro or in vivo comprising the steps of coating a surface with a humanized MN-C3 or MN-C8 antibody, antibody fragment or single chain antibody thereof, contacting the stem cell to the surface and delivering the stem cell to a specific location.

In another aspect, the invention is directed to a method of isolating stem cell comprising the steps of coating a surface with a humanized MN-C3 or MN-C8 antibody, antibody fragment or single chain antibody thereof, and contacting a mixed population of cells to the surface and isolating stem cell.

In another aspect, the invention is directed to a scFv comprising variable domain fragments derived from an antibody that binds to an extracellular domain of MUC1 isoform or cleavage product that is devoid of the tandem repeat domains. The variable domain fragments may be derived from mouse monoclonal antibody MN-E6 (SEQ ID NO:13 and 66) or from the humanized MN-E6 (SEQ ID NO: 39 and 94), or from MN-E6 scFv (SEQ ID NO: 233, 235 and 237). Or, the variable domain fragments may be derived from mouse monoclonal antibody MN-C2 (SEQ ID NO: 119 and 169) or from the humanized MN-C2 (SEQ ID NO: 145 and 195), or from MN-C2 scFv (SEQ ID NO: 239, 241 and 243). Or, the variable domain fragments may be derived from mouse monoclonal antibody MN-C3 (SEQ ID NO: 414 and 459) or from the humanized MN-C3 (SEQ ID NO: 440 and 487), or from MN-C3 scFv (SEQ ID NO: 245, 247 and 249). Or, the variable domain fragments may be derived from mouse monoclonal antibody MN-C8 (SEQ ID NO: 505 and 544) or from the humanized MN-C8 (SEQ ID NO: 526 and 566), or from MN-C8 scFv (SEQ ID NO: 251, 253, 255).

In another aspect, the invention is directed to a method for the treatment of a person diagnosed with, suspected of having or at risk of developing a MUC1 or MUC1* positive cancer involving administering to the person an effective amount of the scFv described above.

In another aspect, the invention is directed to a scFv-Fc construct comprising the scFv as described above. The scFv-Fc may be dimerized. The Fc component may be mutated so that scFv-Fc is monomeric. The mutation may include mutating or deleting hinge region on Fc, making F405Q, Y407R, T366W/L368W, or T364R/L368R mutation or combinations thereof on the Fc represented by SEQ ID NO: 281, 279, 285 and 287.

In another aspect, the invention is directed to a polypeptide comprising at least two different scFv sequences, wherein one of the scFv sequences is a sequence that binds to extracellular domain of MUC1 isoform or cleavage product that is devoid of the tandem repeat domains. The polypeptide may bind to

(i) PSMGFR region of MUC1;

(ii) PSMGFR peptide;

(iii) a peptide having amino acid sequence of SNIKFRPGSVVVQLTLAFREGTINVHDVETQFNQYKTEAASRY (SEQ ID NO:620);

(iv) a peptide having amino acid sequence of VQLTLAFREGTINVHDVETQFNQYKTEAASRY (SEQ ID NO:621);

(v) a peptide having amino acid sequence of VQLTLAFREGTINVHDVETQFNQY (SEQ ID NO:622); or

(vi) a peptide having amino acid sequence of SNIKFRPGSVVVQLTLAFREGTIN (SEQ ID NO:623).

The polypeptide may bind to a receptor on an immune cell, such as T cell, and in particular, CD3 on T-cell.

In another aspect, the invention is directed to a method of detecting presence of a cell that expresses MUC1* aberrantly, comprising contacting a sample of cells with the scFv-Fc described above and detecting for the presence of the binding of scFv-Fc to the cell. The cell may be cancer cell.

In another aspect, the invention is directed to a method for testing a subject's cancer for suitability of treatment with a composition comprising portions of the variable regions of MN-E6, MN-C2, MN-C3 or MN-C8, comprising the steps of contacting a bodily specimen from the patient with the corresponding MN-E6 scFv-Fc, MN-C2 scFv-Fc, MN-C3 scFv-Fc or MN-C8 scFv-Fc.

In another aspect, the invention is directed to a method of treating a subject suffering from a disease comprising, exposing T cells from the subject to MUC1* peptides wherein through various rounds of maturation, T cells develop MUC1* specific receptors, creating adapted T cells, and expanding and administering the adapted T cells to the donor patient who is diagnosed with, suspected of having, or is at risk of developing a MUC1* positive cancer.

In one aspect, the invention may be directed to an immune cell transfected or transduced with a cleavage enzyme for the treatment of cancer. The cancer may be a MUC1 positive cancer. The immune cell may be a T cell. The immune cell may be derived from the patient to be treated. The cleavage enzyme may be an MMP or ADAM family member. The cleavage enzyme may be MMP2, MMP9, MMP3, MMP14, ADAM17, ADAM28, or ADAM TS16.

In another aspect, the invention may be directed to an immune cell transfected or transduced with a CAR wherein its extra cellular domain comprises an antibody scFv that binds to the extra cellular domain of a MUC1 molecule that is devoid of the tandem repeats.

In another aspect, the invention may be directed to an immune cell transfected or transduced with a cleavage enzyme for the treatment of cancer. The cancer may be a MUC1 positive cancer. The immune cell may be a T cell. The immune cell may be an NK cell. The cleavage enzyme may be any enzyme that cleaves MUC1 such that the tandem repeat domain is separated from the transmembrane domain. Such cleavage enzymes include but are not limited to MMP1, MMP2, MMP3, MMP7, MMP8, MMP9, MMP11, MMP12, MMP13, MMP14, MMP16, ADAM9, ADAM10, ADAM17, ADAM 19, ADAMTS16, ADAM28 or a catalytically active fragment thereof. The immune cell may be further transfected or transduced with an activator of the cleavage enzyme. The cleavage enzyme may be without limitation, MMP2 or MMP9 or ADAM17, and the activator of cleavage enzymes MMP2 and MMP9 may be MMP14 and MMP3, respectively. The nucleic acid encoding the cleavage enzyme may be linked to an inducible promoter. The expression of the cleavage enzyme may be induced by an event that occurs specifically when the immune cell mounts an immune response to a target tumor cell. In one aspect of the invention, the cleavage enzyme cleaves MUC1 such that the cleavage product is recognized by an antibody that specifically recognizes cleaved MUC1 on cancerous tissues. In one aspect, the antibody that specifically recognizes cleaved MUC1 on cancerous tissues would bind to cancerous tissues at least two-times more than it binds to healthy tissues where T cells normally traffic.

In another aspect, the invention may be directed to an immune cell transfected or transduced with a CAR comprising an antibody fragment, and a cleavage enzyme for the treatment of cancer. The cancer may be a MUC1 positive cancer. The immune cell may be a T cell. The antibody fragment of the CAR on the T cell may direct the cell to a MUC1* positive tumor. The antibody fragment of the CAR on the T cell may recognize a form of MUC1 after it is cleaved by that specific cleavage enzyme. The antibody fragment of the CAR may be derived from MNC2 or MNE6 and the cleavage enzyme may be MMP9, MMP2, or ADAM17 or an activated form of MMP9, MMP2 or ADAM17. The immune cell may be further transfected or transduced with an activator of the cleavage enzyme. The cleavage enzyme maybe MMP2 or MMP9 or ADAM17, and an activator of cleavage enzymes MMP2 and MMP9 may be MMP14 and MMP3, respectively. The nucleic acid encoding the cleavage enzyme may be linked to an inducible promoter. The expression of the cleavage enzyme may be induced by an event that occurs specifically when the immune cell mounts an immune response to a target tumor cell. The antibody fragment may recognize a form of MUC1 or MUC1* that is created when the cleavage enzyme cleaves MUC1 or MUC1*. Expression of the cleavage enzyme by the inducible promoter may be induced when the antibody fragment of the CAR engages or binds to a MUC1 or MUC1* on the tumor.

In another aspect, the invention is directed to a method of treating cancer in a patient comprising administering to the patient the immune cell of any of the above, in combination with a checkpoint inhibitor.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIGS. 1A-1D show cell growth assay graphs of MUC1* positive cells treated with either bivalent ‘bv’ anti-MUC1* antibody, monovalent ‘my’ or Fab, NM23-H1 dimers or NME7-AB. Bivalent anti-MUC1* antibodies stimulate growth of cancer cells whereas the monovalent Fab inhibits growth (A, B). Classic bell-shaped curve indicates ligand induced dimerization stimulates growth. Dimeric NM23-H1, aka NME1, stimulates growth of MUC1* positive cancer cells but siRNA to suppress MUC1 expression eliminate its effect (C). NME7-AB also stimulates the growth of MUC1* positive cells (D).

FIGS. 2A-2F show results of ELISA assays. MUC1* peptides PSMGFR, PSMGFR minus 10 amino acids from the N-terminus aka N-10, or PSMGFR minus 10 amino acids from the C-terminus, aka C-10 are immobilized on the plate and the following are assayed for binding: NME7-AB (A), MN-C2 monoclonal antibody (B), MN-E6 monoclonal antibody (C), or dimeric NME1 (D). These assays show that NME1, NME7-AB and monoclonal antibodies MN-C2 and MN-E6 all require the first membrane proximal 10 amino acids of the MUC1* extracellular domain to bind. MUC1* peptides PSMGFR minus 10 amino acids from the N-terminus aka N-10, or PSMGFR minus 10 amino acids from the C-terminus, aka C-10, are immobilized on the plate and the following are assayed for binding: MN-C3 (E) and MN-C8 (F).

FIGS. 3A-3C show results of competitive ELISA assays. The PSMGFR MUC1* peptide is immobilized on the plate and dimeric NM23-H1, aka NME1, is added either alone or after the MN-E6 antibody has been added (A). The same experiment was performed wherein NM23-H7, NME7-AB, is added alone or after MN-E6 has been added (B). Results show that MN-E6 competitively inhibits the binding of MUC1* activating ligands NME1 and NME7. In a similar experiment (C), PSMGFR or PSMGFR minus 10 amino acids from the N-terminus, aka N-10, is immobilized on the plate. Dimeric NM23-H1 is then added. Anti-MUC1* antibodies MN-E6, MN-C2, MN-C3 or MN-C8 are then tested for their ability to compete off the NM23-H1. Results show that although all three antibodies bind to the PSMGFR peptides, MN-E6 and MN-C2 competitively inhibit binding of the MUC1* activating ligands.

FIGS. 4A-4F show FACS scans of anti-MUC1* antibodies binding specifically to MUC1* positive cancer cells and MUC1* transfected cells but not MUC1* or MUC1 negative cells. ZR-75-1, aka 1500, MUC1* positive breast cancer cells were stained with 1:2 or 1:10 dilutions of the 1.5 ug/ml humanized MN-C2. After two washes, cells were stained with secondary antibody, Anti-Penta-His antibody at conjugated to Alexa 488 (Qiagen) dilutions of 1:200 (A), 1:50 (B), or 1:10 (C) to detect the 6× His tag on the huMN-C2 scFv. Flow cytometric analysis revealed a concentration-dependent shift of a subset of cells, indicating specific binding, which is unseen in the absence of the MN-C2 scFv (A-C). In another case, MN-E6 was used to stain MUC1 negative HCT-116 colon cancer cells transfected with the empty vector, single cell clone #8 (D), HCT-116 colon cancer cells transfected with MUC1* single cell clone #10 (E), or ZR-75-1, aka 1500, MUC1* positive breast cancer cells. As the FACS scans show, both MN-C2 and MN-E6 only stain MUC1* positive cells and not MUC1 or MUC1* negative cells.

FIG. 5 shows a graph of an ELISA in which surface is coated with either the MUC1* PSMGFR peptide or a control peptide. Humanized MN-C2 scFv is then incubated with the surface, washed and detected according to standard methods. The ELISA shows that the huMN-C2 scFv binds to the MUC1* peptide with an EC-50 of about 333 nM.

FIGS. 6A-6B show graphs of cancer cell growth inhibition by MUC1* antibody variable region fragment humanized MN-C2 scFv. hMN-C2 scFv potently inhibited the growth of ZR-75-1, aka 1500, MUC1* positive breast cancer cells (A) and T47D MUC1* positive breast cancer cells (B) with approximately the same EC-50 as the in vitro ELISAs.

FIGS. 7A-7B show graphs of tumor growth in immune compromised mice that have been implanted with human tumors then treated with anti-MUC1* antibody MN-E6 Fab or mock treatment. Female nu/nu mice implanted with 90-day estrogen pellets were implanted with 6 million T47D human breast cancer cells that had been mixed 50/50 with Matrigel. Mice bearing tumors that were at least 150 mm³ and had three successive increases in tumor volume were selected for treatment. Animals were injected sub cutaneously twice per week with 80 mg/kg MN-E6 Fab and an equal number of mice fitting the same selection criteria were injected with vehicle alone (A). Male NOD/SCID mice were implanted with 6 million DU-145 human prostate cancer cells that had been mixed 50/50 with Matrigel. Mice bearing tumors that were at least 150 mm³ and had three successive increases in tumor volume were selected for treatment. Animals were injected sub-cutaneously every 48 hours with 160 mg/kg MN-E6 Fab and an equal number of mice fitting the same selection criteria were injected with vehicle alone (B). Tumors were measured independently by two researchers twice per week and recorded. Statistics were blindly calculated by independent statistician, giving a P value of 0.0001 for each. Anti-MUC1* Fab inhibited breast cancer growth and prostate cancer growth. Treatment had no effect on weight, bone marrow cell type or number.

FIG. 8 is a graph of an ELISA assay showing differing levels of expression of humanized MN-E6 anti-MUC1* antibody depending on whether the light chain was kappa or lambda and whether the variable portion was fused to a human IgG or IgG2.

FIG. 9 is a graph of an ELISA assay comparing the binding of the parent mouse MN-E6 antibody to the humanized versions of the MN-E6 antibody to a surface presenting the PSMGFR peptide derived from the MUC1* extracellular domain.

FIG. 10 is a graph of an ELISA assay showing differing levels of expression of humanized MN-C2 anti-MUC1* antibody depending on whether the light chain was kappa or lambda and whether the variable portion was fused to a human IgG or IgG2.

FIG. 11 is a graph of an ELISA assay comparing the binding of the parent mouse MN-C2 antibody to the humanized versions of the MN-C2 antibody to a surface presenting the PSMGFR peptide derived from the MUC1* extracellular domain.

FIG. 12 is a graph of an ELISA assay showing binding of humanized single chain (scFv) MN-C2 and MN-E6 antibodies binding to a surface presenting the PSMGFR peptide derived from the MUC1* extracellular domain.

FIGS. 13A-13C show FPLC traces of the purification of MN-E6 scFv-Fc fusion protein that was grown in low IgG FBS over a Protein A affinity column. A) is the trace of the flow through. B) is the trace of the elution. C) shows the purified protein on a reducing or non-reducing gel.

FIGS. 14A-14B show photographs of SDS-PAGE characterization of purified MN-E6 scFv-Fc fusion proteins on a non-reducing gels, wherein the Fc portion that was fused to the MN-E6 was either wild type (wt) or mutated as follows: A) F405Q, Y407R, T394D; B) T366W/L368W, T364R/L368R, T366W/L368W or T364R/L368R. Fc mutants F405Q, Y407R, T366W/L368W, T364R/L368R, T366W/L368W and T364R/L368R all favored monomer over dimer formation. The reference construct amino acid sequence for the indicated mutations is SEQ ID NO:273.

FIGS. 15A-15B show FPLC traces of the purification of MN-E6 scFv-Fc Y407Q fusion protein that was grown in low IgG FBS over a Protein A affinity column. A) is the trace of the flow through. B) is the trace of the elution. The protein was further purified by size exclusion over an S200 column (C). (D) is a photograph of an SDS-PAGE gel showing which fractions had a predominance of monomer. The reference construct amino acid sequence for the indicated mutations is SEQ ID NO:273.

FIG. 16 shows a photograph of SDS-PAGE characterization of purified MN-E6 scFv-Fc-mutant fusion proteins on a non-reducing gel, wherein the Fc portion that was fused to the MN-E6 scFv was either wild type (wt) or mutated by elimination of the hinge region, ‘DHinge’, of the Fc or elimination of the hinge region of the Fc and also bearing the Y407R mutation. All the Fc mutants favored monomer over dimer formation. The reference construct amino acid sequence for the indicated mutations is SEQ ID NO:273.

FIGS. 17A-17C. A and B show photograph of non-reducing SDS-PAGE characterization of large scale expression and purification of MN-E6 scFv-Fc hingeless mutant, showing that it is a monomer. FPLC characterization and purification of MN-E6 scFv-Fc hingeless mutant is shown (C).

FIGS. 18A-18C show photographs of the SDS-PAGE characterization of the purified MN-C3 scFv-Fc fusion protein on a non-reducing gel (A) or a reducing gel (B). The protein was purified by size exclusion. The FPLC trace is shown (C).

FIGS. 19A-19B show photographs of Native gels of MN-C3 or MN-E6 Fabs, scFv, scFv-Fc, wherein the Fc portion is wild type or mutants that prefer or are exclusively monomers. Native gels show that the Y407R Fc mutation (A) and the double mutant Y407R and a deleted hinge (B) favor monomer over dimer the best. Note that proteins are loaded onto a gel at much higher concentrations than typical use concentrations. The dimer formation of other Fc mutants may only reflect the fact that loading concentration is very high.

FIG. 20 shows a graph of an ELISA wherein the surface was immobilized with either PSMGFR peptide, PSMGFR minus 10 amino acids from the N-terminus or minus 10 amino acids from the C-terminus. The hu MN-E6 scFv-Fc bound to the PSMGFR peptide and to the PSMGFR N-10 peptide but not to the PSMGFR C-10 peptide. The parent MN-E6 antibody and the humanized MN-E6 require the C-terminal 10 amino acids of PSMGFR for binding.

FIGS. 21A-21B show an ELISA graph of several anti-MUC1* scFv-Fc fusion proteins wherein the Fc region has been eliminated or mutated. Shown are hu MN-E6 scFv-Fc-wt, hu MN-E6 scFv-Fc-Y407R, hu MN-E6 scFv-Fc-hingeless, and hu MN-E6 scFv-Fc- Y407R-hingeless. All mutants bind to the PSMGFR peptide of the MUC1* extracellular domain (A). An ELISA graph of several anti-MUC1* scFv-Fc fusion proteins wherein the Fc region is either wild type or mutated. Shown are hu MN-E6 scFv-Fc-wt, hu MN-E6 scFv-Fc-hingeless, and hu MN-C3 scFv-Fc is shown (B). All bind to the PSMGFR peptide of the MUC1* extracellular domain.

FIGS. 22A-22C show graphs of ELISAs wherein the assay plate surface was immobilized with either PSMGFR peptide, PSMGFR minus 10 amino acids from the N-terminus or minus 10 amino acids from the C-terminus. The MN-C3 antibody variants were then assayed for binding to the various MUC1* peptides. A) Purified mouse monoclonal MN-C3 antibody; B) Impure humanized MN-C3 antibody; and C) the humanized MN-C3 scFv-Fc. ELISAs show binding to the PSMGFR peptide as well as to certain deletion peptides.

FIG. 23 shows a graph of an ELISA assay that quantifies the binding of humanized MN-E6 scFv-Fc-delta hinge, aka Dhinge or hingeless, and humanized MN-E6 scFv to the MUC1* peptide PSMGFR.

FIG. 24 shows photographs of immunofluorescence experiments in which humanized MN-C2 scFv or MN-E6 scFv specifically binds to MUC1* positive breast cancer cells in an identical concentration dependent manner. A-G: hu MN-C2 scFv binding to T47D breast cancer cells at concentrations indicated. H—N shows the fluorescently labeled scFv and DAPI. O-U: hu MN-E6 scFv binding to T47D breast cancer cells at concentrations indicated. V-B′ shows the fluorescently labeled scFv and DAPI. C′ is the secondary antibody control.

FIGS. 25A-25L show photographs of 1500 MUC1* positive breast cancer cells that have been cultured in normal medium or in the presence of humanized MN-E6 scFv. A-D are bright field images taken at 4× magnification. E-H are calcein fluorescent images taken at 4× magnification. I-L are calcein fluorescent images taken at 10× magnification. A, E, I show control cells cultured in normal RPMI medium. B, F, J show control cells cultured in normal RPMI medium plus a volume of PBS equal to the volume of MN-E6 scFv in PBS that is added to experimental wells. C, G, K show cells cultured in normal RPMI medium plus 500 ug/mL MN-E6 scFv. D, H, L show cells cultured in normal RPMI medium plus 5 ug/mL MN-E6 scFv. The photographs show killing and/or growth inhibition of MUC1* positive cells by MN-E6 scFv at 5 ug/mL and an even greater effect at 500 ug/mL. Images were taken at 96 hours post addition of test molecule.

FIGS. 26A-26L show photographs of 1500 MUC1* positive breast cancer cells that have been cultured in normal medium or in the presence of humanized MN-E6 scFv-Fc Dhinge, which is a hingeless or delta hinge mutant. A-F are bright field images taken at 20× magnification. G-L are calcein fluorescent images taken at 4× magnification. A, G show control cells cultured in normal RPMI medium. B, H show cells cultured in normal RPMI medium plus 100 ug/mL hMN-E6 scFv-Fc Dhinge. C, I show cells cultured in normal RPMI medium plus 50 ug/mL hMN-E6 scFv-Fc Dhinge. D, J show cells cultured in normal RPMI medium plus 5 ug/mL hMN-E6 scFv-Fc Dhinge. E, K show cells cultured in normal RPMI medium plus 0.5 ug/mL hMN-E6 scFv-Fc Dhinge. F, L show cells cultured in normal RPMI medium plus 500 ug/mL of MN-E6 Fab. The photographs show killing and/or growth inhibition of MUC1* positive cells by hMN-E6 scFv-Fc Dhinge 5 ug/mL, an even greater effect at 50 ug/mL and yet an even greater effect at 100 ug/mL. Comparing cell morphology to the control cells, cancer cells grown in MN-E6 Fab or in an effective amount of hMN-E6 scFv-Fc Dhinge, show rounding up of the cells which morphology change occurs before cell death. Images were taken at 96 hours post addition of test molecule.

FIG. 27 shows a graph of the image analysis of the fluorescent images of FIGS. 25 and 26. Image J was used to quantify the number of cells remaining after 96 hours treatment in humanized MN-E6scFv or MN-E6 scFv-Fc-delta hinge, aka Dhinge. The analysis software uses pixel counting and pixel fluorescence intensity to quantify the number of cells in each photograph. Analysis was performed over the entire image 512×512 pixels, 8-bit image. For comparison, the inhibition of mouse monoclonal MN-E6 Fab is also analyzed.

FIGS. 28A-28C show schematics of CAR sequence components.

FIG. 29 is a graph of an experiment measuring IL-2 cytokine secretion by Jurkat T cells that were transduced with a panel of CARs, including MN-E6-CD8-3z, MN-E6-CD8-CD28-3z, MN-E6-CD8-41BB-3z, MN-E6-CD4-CD28-3z and MN-E6-CD4-CD28-41BB-3z, when the CAR T cells were exposed to K562-wt cells or K562 cells that had been transfected with MUC1*.

FIG. 30 is a graph of an experiment measuring IL-2 cytokine secretion by Jurkat T cells that were transduced with a panel of CARs, including MN-E6-CD8-CD28-3z, MN-E6-CD8-41BB-3z, MN-E6-CD4-CD28-3z and MN-E6-CD4-41BB-3z, when the CAR T cells were exposed to K562-wt cells or K562 cells that had been transfected with MUC1*.

FIG. 31 is a graph of an experiment measuring IL-2 cytokine secretion by primary human T cells, isolated from a blood sample, that were transduced with a panel of CARs, including MN-E6-CD8-CD28-3z, MN-E6-CD8-41BB-3z and MN-E6-CD4-41BB-3z, when the CAR T cells were exposed to K562-wt cells or K562 cells that had been transfected with MUC1*.

FIG. 32 is a graph of an experiment measuring interferon-gamma (IFN-g) cytokine secretion by primary human T cells, isolated from a blood sample, that were transduced with a panel of CARs, including MN-E6-CD8-CD28-3z and MN-E6-CD4-41BB-3z, when the CAR T cells were exposed to K562-wt cells or K562 cells that had been transfected with MUC1*.

FIG. 33 is a graph of an experiment measuring interferon-gamma (IFN-g) cytokine secretion by primary human T cells, isolated from a blood sample, that were transduced with a panel of CARs, including MN-E6-CD8-CD28-3z, MN-E6-CD8-41BB-3z and MN-E6-CD8-CD28-41BB-3z, when the CAR T cells were exposed to K562-wt cells, K562 cells that had been transfected with MUC1*, or MUC1* positive cancer cells of prostate cancer, breast cancer or pancreatic cancer.

FIG. 34 is a graph of an experiment measuring target cell death when primary human T cells, isolated from a blood sample, that were transduced with a panel of CARs, including MN-E6-CD8-CD28-3z, MN-E6-CD8-41BB-3z and MN-E6-CD4-41BB-3z, when the CAR T cells were exposed to K562-wt cells or K562 cells that had been transfected with MUC1*. The ratio of T cells to target cells was 1:1 and the cells were co-cultured for 24 hours.

FIGS. 35A-35B are graphs of FACS measuring a time course of target cell survival from Day 1 to Day 3. Primary human T cells, isolated from a blood sample, were transduced with a panel of CARs, including humanized MN-E6-CD8-3z, MN-E6-CD8-CD28-3z, MN-E6-CD8-41BB-3z and MN-E6-CD8-CD28-41BB-3z. The CAR T cells were then exposed to K562-wt cells that naturally express low levels of MUC1*, or K562 cells that had been transfected with MUC1* high. The ratio of MUC1* targeting CAR T cells to target cells was either 1:1, 10:1, or 20:1. Surviving cells were detected and measured at Day 1 (A) or Day 3 (B).

FIG. 36 is a graph of FACS measurements of target cell survival at Day 3 of co-culture experiment. Primary human T cells were transduced with a panel of CARs, including humanized MN-E6-CD8-3z, MN-E6-CD8-CD28-3z, MN-E6-CD8-41BB-3z and MN-E6-CD8-CD28-41BB-3z. The CAR T cells were then exposed to MUC1* positive T47D breast cancer cells or MUC1* positive 1500 aka ZR-75-1 breast cancer cells. The ratio of MUC1* targeting CAR T cells to target cells was either 1:1 or 10:1. As can be seen from the graph, T cells transduced with a MUC1* targeting CAR have a much greater killing effect on MUC1* cancer cells than the untransduced control T cells. In addition, the killing effect is much greater when the ratio of T cells:target cells is increased.

FIG. 37 is a graph of FACS measurements of target cell survival at Day 1 of co-culture experiment. Primary human T cells were transduced with a panel of CARs, including humanized MN-E6-CD8-41BB-3z, MN-E6-CD4-41BB-3z, and MN-E6-CD8-CD28-41BB-3z. The CAR T cells were then exposed to the following MUC1* positive cancer cells: T47D breast cancer; capan2 pancreatic cancer; or DU-145 prostate cancer. The ratio of MUC1* targeting CAR T cells to target cells was 5:1. As can be seen from the graph, T cells transduced with a MUC1* targeting CAR have a much greater killing effect on MUC1* cancer cells than the untransduced control T cells. Note that the measurements were taken after 24 hours with only a 5:1 T cell to target cell ratio. Also note that MUC1* targeting CARs that have a CD4 extracellular domain-transmembrane-cytoplasmic tail work equally well as CD8 constructs.

FIG. 38 is a graph of FACS measurements of target cell survival at Day 3 of co-culture experiment. Primary human T cells were transduced with a panel of CARs, including humanized MN-E6-CD8-41BB-3z, MN-E6-CD4-41BB-3z, and MN-E6-CD8-CD28-41BB-3z. The CAR T cells were then exposed to the following MUC1* positive cancer cells: K562 leukemia cells transfected with MUC1*; T47D breast cancer; 1500 aka ZR-75-1 breast cancer cells; or CAPAN-2 pancreatic cancer cells. In addition to the untransduced T cell controls, the assay was performed on PC3 MUC1* negative prostate cancer cells. The ratio of MUC1* targeting CAR T cells to target cells was 1:1. As can be seen from the graph, T cells transduced with a MUC1* targeting CAR have a much greater killing effect on MUC1* cancer cells than the untransduced control T cells. In addition, the killing effect is specific for MUC1* positive cells. Note that MUC1* targeting CARs that have a CD4 extracellular domain-transmembrane-cytoplasmic tail work equally well as CD8 constructs.

FIG. 39 is a graph of FACS measurements of CAR T cell expansion over 24 hours in co-culture with target cells at a ratio of 5:1 CAR T cells to target cells. The primary human T cells were transduced with a panel of CARs, including humanized MN-E6-CD8-41BB-3z, MN-E6-CD4-41BB-3z, and MN-E6-CD8-CD28-41BB-3z. The CAR T cells were co-cultured with MUC1* positive T47D breast cancer cells, MUC1* positive Capan pancreatic cancer cells, and MUC1-negative cells HCT-116 colon cancer cells and HEK-293 human embryonic kidney cells. As can be seen from the graph, the CAR T population is increased in the presence of MUC1* positive cells.

FIG. 40 shows a photograph of a Western blot of MUC1* targeting CARs. From 1 to 9 are: 1. E6scFv-Fc-8-41BB-CD3z (Human Fc as hinge region with CD8 TM); 2: E6scFv-FcH-8-41BB-CD3z (Human Fc hingeless as hinge region with CD8 TM) 3: E6scFv-Fc-4-41BB-CD3z (Human Fc as hinge region with CD4 TM) 4: E6scFv-FcH-4-41BB-CD3z (Human Fc as hingeless hinge region with CD4 TM) 5: E6scFv-IgD-8-41BB-CD3z (hinge region from human IgD with CD8 TM) 6: E6scFv-IgD-4-41BB-CD3z (hinge region from human IgD with CD4 TM) 7: E6scFv-X4-8-41BB-CD3z (Long flexible linker as hinge region with CD8 TM) 8: E6scFv-X4-4-41BB-CD3z (Long flexible linker as hinge region with CD4 TM) 9: E6scFv-8-4-41BB-CD3z (Hinge region from CD8 and CD4 a with CD4 TM).

FIG. 41 shows graphs of FACS scans of T47D breast cancer cells co-cultured with human T cells that were transduced with MN- E6scFv-Fc-8-41BB-CD3z, MN-E6scFv-FcH-8-41BB-CD3z, MN-E6scFv-Fc-4-41BB-CD3z, MN-E6scFv-IgD-8-41BB-CD3z, MN-E6scFv-X4-8-41BB-CD3z and MN-E6scFv-X4-4-41BB-CD3z. T cells and cancer cells were co-cultured at a 1:1 ratio for 48 hours. T cell counts were normalized to an average of all untransduced T cells and target cells were normalized to each specific cell type when co-cultured with untransduced T cells. The graph shows that when the CAR T cells are co-cultured with MUC1* positive cancer cells, the T cell population expands and the targeted cancer cell population decreases.

FIG. 42 shows graphs of FACS scans of T47D breast cancer cells, Capan-2 pancreatic cancer cells, K562-MUC1* transfected cells, and K562-wt cells that were co-cultured with human T cells that were transduced with MN- E6scFv-Fc-8-41BB-CD3z, MN-E6scFv-FcH-8-41BB-CD3z, MN-E6scFv-Fc-4-41 BB-CD3z, MN-E6scFv-IgD-8-41BB-CD3z, MN-E6scFv-X4-8-41BB-CD3z and MN-E6scFv-X4-4-41BB-CD3z. T cells and cancer cells were co-cultured at a 1:1 ratio for 48 hours. T cell counts were normalized to an average of all untransduced T cells and target cells were normalized to each specific cell type when co-cultured with untransduced T cells. The graph shows that when the CAR T cells are co-cultured with MUC1* positive cancer cells, the T cell population expands and the targeted cancer cell population decreases.

FIGS. 43A-43J. A,B are photographs of breast cancer tissue arrays. A) was stained with VU4H5 which recognizes MUC1-FL (full length); B) was stained with mouse monoclonal antibody MN-C2 which recognizes cancerous MUC1*. Following automated staining (Clarient Diagnostics), the tissue staining was scored using Allred scoring method which combines an intensity score and a distribution score. C,D,E,F are color coded graphs showing the score calculated for MUC1 full-length staining for each patient's tissue. G,H,I,J are color coded graphs showing the score calculated for MUC1* staining for each patient's tissue.

FIGS. 44A-44J. A,B are photographs of breast cancer tissue arrays. A) was stained with VU4H5 which recognizes MUC1-FL (full length); B) was stained with mouse monoclonal antibody MN-C2 which recognizes cancerous MUC1*. Following automated staining (Clarient Diagnostics), the tissue staining was scored using Allred scoring method which combines an intensity score and a distribution score. C,D,E,F are color coded graphs showing the score calculated for MUC1 full-length staining for each patient's tissue. G,H,I,J are color coded graphs showing the score calculated for MUC1* staining for each patient's tissue.

FIGS. 45A-45H show photographs of normal breast and breast cancer tissues stained with humanized MN-E6-scFv-Fc biotinylated anti-MUC1* antibody at 2.5 ug/mL, then stained with a secondary streptavidin HRP antibody. A) is a normal breast tissue. B-D are breast cancer tissues from patients as denoted in the figure. E-H are photographs of the corresponding serial sections that were stained with the secondary antibody alone.

FIGS. 46A-46F show photographs of normal breast and breast cancer tissues stained with humanized MN-E6-scFv-Fc biotinylated anti-MUC1* antibody at 2.5 ug/mL, then stained with a secondary streptavidin HRP antibody. A) is a normal breast tissue. B-C are breast cancer tissues from patients as denoted in the figure. D-F are photographs of the corresponding serial sections that were stained with the secondary antibody alone.

FIGS. 47A-47H show photographs of breast cancer tissues stained with MN-E6 anti-MUC1* antibody at 10 ug/mL, then stained with a rabbit anti mouse secondary HRP antibody. A-D are breast cancer tissues from patient #300. E-H are breast cancer tissues from metastatic patient #291.

FIGS. 48A-48F show photographs of normal lung and lung cancer tissues stained with humanized MN-E6-scFv-Fc biotinylated anti-MUC1* antibody at 2.5 ug/mL, then stained with a secondary streptavidin HRP antibody. A) is a normal lung tissue. B,C are lung cancer tissues from patients as denoted in the figure. D-F are photographs of the corresponding serial sections that were stained with the secondary antibody alone.

FIGS. 49A-49F show photographs of normal lung and lung cancer tissues stained with humanized MN-E6-scFv-Fc biotinylated anti-MUC1* antibody at 2.5 ug/mL, then stained with a secondary streptavidin HRP antibody. A) is a normal lung tissue. B,C are lung cancer tissues from patients as denoted in the figure. D-F are photographs of the corresponding serial sections that were stained with the secondary antibody alone.

FIGS. 50A-50F show photographs of normal lung and lung cancer tissues stained with humanized MN-E6-scFv-Fc biotinylated anti-MUC1* antibody at 25 ug/mL, then stained with a secondary streptavidin HRP antibody. A) is a normal lung tissue. B,C are lung cancer tissues from patients as denoted in the figure. D-F are photographs of the corresponding serial sections that were stained with the secondary antibody alone.

FIGS. 51A-51F show photographs of normal lung and lung cancer tissues stained with humanized MN-E6-scFv-Fc biotinylated anti-MUC1* antibody at 25 ug/mL, then stained with a secondary streptavidin HRP antibody. A) is a normal lung tissue. B,C are lung cancer tissues from patients as denoted in the figure. D-F are photographs of the corresponding serial sections that were stained with the secondary antibody alone.

FIGS. 52A-52D show photographs of normal small intestine and cancerous small intestine tissues stained with humanized MN-E6-scFv-Fc biotinylated anti-MUC1* antibody at 5 ug/mL, then stained with a secondary streptavidin HRP antibody. A) is a normal small intestine tissue. B) is small intestine cancer from patient as denoted in the figure. C,D are photographs of the corresponding serial sections that were stained with the secondary antibody alone.

FIGS. 53A-53H show photographs of normal small intestine tissues stained with humanized MN-E6-scFv-Fc anti-MUC1* antibody at 50 ug/mL, then stained with a secondary goat-anti-human HRP antibody. A-D are normal small intestine tissue. E-H are photographs of the corresponding serial sections that were stained with the secondary antibody alone.

FIGS. 54A-54H show photographs of cancerous small intestine tissues stained with humanized MN-E6-scFv-Fc anti-MUC1* antibody at 50 ug/mL, then stained with a secondary goat-anti-human HRP antibody. A-D are cancerous small intestine tissue from a patient as denoted in figure. E-H are photographs of the corresponding serial sections that were stained with the secondary antibody alone.

FIGS. 55A-55H show photographs of cancerous small intestine tissues stained with humanized MN-E6-scFv-Fc anti-MUC1* antibody at 50 ug/mL, then stained with a secondary goat-anti-human HRP antibody. A-D are cancerous small intestine tissue from a patient as denoted in figure. E-H are photographs of the corresponding serial sections that were stained with the secondary antibody alone.

FIGS. 56A-56H show photographs of normal colon tissues stained with humanized MN-E6-scFv-Fc anti-MUC1* antibody at 50 ug/mL, then stained with a secondary goat-anti-human HRP antibody. A-D are normal colon. E-H are photographs of the corresponding serial sections that were stained with the secondary antibody alone.

FIGS. 57A-57H show photographs of colon cancer tissues stained with humanized MN-E6-scFv-Fc anti-MUC1* antibody at 50 ug/mL, then stained with a secondary goat-anti-human HRP antibody. A-D are colon cancer tissue from a metastatic patient as denoted in figure. E-H are photographs of the corresponding serial sections that were stained with the secondary antibody alone.

FIGS. 58A-58H show photographs of colon cancer tissues stained with humanized MN-E6-scFv-Fc anti-MUC1* antibody at 50 ug/mL, then stained with a secondary goat-anti-human HRP antibody. A-D are colon cancer tissue from a Grade 2 patient as denoted in figure. E-H are photographs of the corresponding serial sections that were stained with the secondary antibody alone.

FIGS. 59A-59H show photographs of colon cancer tissues stained with humanized MN-E6-scFv-Fc anti-MUC1* antibody at 50 ug/mL, then stained with a secondary goat-anti-human HRP antibody. A-D are colon cancer tissue from a metastatic patient as denoted in figure. E-H are photographs of the corresponding serial sections that were stained with the secondary antibody alone.

FIGS. 60A-60H show photographs of prostate cancer tissues stained with humanized MN-E6-scFv-Fc anti-MUC1* antibody at 50 ug/mL, then stained with a secondary goat-anti-human HRP antibody. A-D are prostate cancer tissue from a patient as denoted in figure. E-H are photographs of the corresponding serial sections that were stained with the secondary antibody alone.

FIGS. 61A-61H show photographs of prostate cancer tissues stained with humanized MN-E6-scFv-Fc anti-MUC1* antibody at 50 ug/mL, then stained with a secondary goat-anti-human HRP antibody. A-D are prostate cancer tissue from a patient as denoted in figure. E-H are photographs of the corresponding serial sections that were stained with the secondary antibody alone.

FIGS. 62A-62H show photographs of prostate cancer tissues stained with humanized MN-E6-scFv-Fc anti-MUC1* antibody at 50 ug/mL, then stained with a secondary goat-anti-human HRP antibody. A-D are prostate cancer tissue from a patient as denoted in figure. E-H are photographs of the corresponding serial sections that were stained with the secondary antibody alone.

FIG. 63 shows fluorescence activated cell sorting (FACS) measurements of human CD34+ bone marrow cells stained with anti-MUC1* monoclonal antibodies MNC3 or MNE6 or an isotype control antibody. The histogram of the FACS assay and the bar graph showing the data show that the MUC1* positive cells of the bone marrow are recognized by one anti-MUC1* antibody, MNC3 but not by another MNE6.

FIG. 64 shows photographs of DU145 prostate cancer cells or T47D breast cancer cells that have been treated with either the Fab of anti-MUC1* antibody MNC2, MNE6, MNC3 or MNC8. The images show that cancer specific antibodies MNC2 and MNE6 effectively kill prostate and breast cancer cells while the monoclonal antibodies MNC3 and MNC8 do not.

FIG. 65 shows a graph of a PCR experiment comparing expression of a wide range of cleavage enzymes expressed in different cells lines, wherein the values have been normalized to those expressed in breast cancer cell line T47D. Cell lines that are compared are prostate cancer cell line DU145, HCT-MUC1-41TR that is a MUC1 negative colon cancer cell line transfected with a MUC1 whose extracellular domain is truncated after 41 tandem repeat units and that is not cleaved to the MUC1* form, T47D breast cancer cell line and CD34+ bone marrow cells.

FIG. 66 shows the graph of the PCR experiment of FIG. 65 but with the Y-axis maximum set to 5.

FIGS. 67A-67B show a graph of a FACS experiment wherein the effect of a panel of cleavage enzyme inhibitors is assessed for breast cancer cell line T47D. FIG. 67A shows the percentage of cells that test positive for full-length MUC1 antibody VU4H5 or for anti-MUC1* monoclonal antibody MNC2. FIG. 67B shows the mean fluorescence intensity of cells probed with antibody VU4H5 versus MNC2. As can be seen, TAPI-1 inhibitor and MMP2/9 V inhibitor inhibited cleavage of MUC1.

FIGS. 68A-68B show graphs of a FACS experiment wherein the effect of a panel of cleavage enzyme inhibitors is assessed for prostate cancer cell line DU145. FIG. 68A shows the percentage of cells that test positive for full-length MUC1 antibody VU4H5 or for anti-MUC1* monoclonal antibody MNC2. FIG. 68B shows the mean fluorescence intensity of cells probed with antibody VU4H5 versus MNC2. As can be seen, none of the cleavage enzyme inhibitors had an effect on MUC1 cleavage.

FIGS. 69A-69B show photographs of serial sections of a breast cancer array that was probed with either full-length antibody VU4H5, FIG. 69A, or anti-MUC1* antibody MNC2, FIG. 69B. The Allred score for each tissue specimen is shown in the graphs below each array photograph, FIG. 69C-69D. The percentage of each array that did not stain at all with the antibody or stained weakly, medium, or strong is graphed as a pie chart and shown in FIG. S7E-S7F.

FIGS. 70A-70F show photographs of a triple negative breast cancer array stained with anti-MUC1* antibody huMNC2scFv. The first score shown is the Allred score and the second is the tumor grade. The percentage of the array that scored zero, weak, medium or strong is graphed as a pie chart. FIG. 70A shows the pie chart of score of anti-MUC1* antibody staining. FIG. 70B shows a photograph of the array stained with the antibody. FIGS. 70C-70D show magnified photographs of two of the breast cancer specimens from the array. FIGS. 70C-70D show more magnified photographs of the portion of the specimen that is marked by a box.

FIGS. 71A-71F show photographs of an ovarian cancer array stained with anti-MUC1* antibody huMNC2scFv. The first score shown is the Allred score and the second is the tumor grade. The percentage of the array that scored zero, weak, medium or strong is graphed as a pie chart. FIG. 71A shows the pie chart of score of anti-MUC1* antibody staining. FIG. 71B shows a photograph of the array stained with the antibody. FIGS. 71C-71D show magnified photographs of two of the breast cancer specimens from the array. FIGS. 71C-71D show more magnified photographs of the portion of the specimen that is marked by a box.

FIGS. 72A-72F show photographs of a pancreatic cancer array stained with anti-MUC1* antibody huMNC2scFv. The first score shown is the Allred score and the second is the tumor grade. The percentage of the array that scored zero, weak, medium or strong is graphed as a pie chart. FIG. 72A shows the pie chart of score of anti-MUC1* antibody staining. FIG. 72B shows a photograph of the array stained with the antibody. FIGS. 72C-72D show magnified photographs of two of the breast cancer specimens from the array. FIGS. 72C-72D show more magnified photographs of the portion of the specimen that is marked by a box.

FIGS. 73A-73F show photographs of a lung cancer array stained with anti-MUC1* antibody huMNC2scFv. The first score shown is the Allred score and the second is the tumor grade. The percentage of the array that scored zero, weak, medium or strong is graphed as a pie chart. FIG. 73A shows the pie chart of score of anti-MUC1* antibody staining. FIG. 73B shows a photograph of the array stained with the antibody. FIGS. 73C-73D show magnified photographs of two of the breast cancer specimens from the array. FIGS. 73C-73D show more magnified photographs of the portion of the specimen that is marked by a box.

FIGS. 74A-74I show photographs of normal tissues stained with anti-MUC1* antibody huMNC2scFv.

FIGS. 75A-75P show photographs of a CAR T co-culture assay in which the targeting antibody fragment of the CAR is huMNC2scFv wherein CAR44 has a CD8 transmembrane domain, followed by 41BB-3zeta and CAR50 has a CD4 transmembrane domain, followed by 41BB-3zeta. The target cancer cells are: HCT-FLR which is HCT-116 cells transfected with MUC1*₄₅ and HCT-MUC1-41TR, which is a stable single cell clone HCT-116 cell line that expresses MUC1 with an extracellular domain truncated after 41 tandem repeats and that does not get cleaved to the MUC1* form on its own. The HCT-MUC1-41TR cancer cells were also incubated with conditioned media from cells transfected with MMP9 or ADAM17 before co-culture with the CAR T cells. Conditioned media of the MMP9 or ADAM17 expressing cells were also incubated with APMA which is an activator of those cleavage enzymes. The images shown are an overlay of the 4× bright field image and the fluorescent image of the same showing cancer cells dyed with a red CMTMR lipophilic dye. FIGS. 75A, 75E, 75I, 75M show photographs of cells co-cultured with untransduced human T cells.

FIGS. 75B, 75F, 75J, 75N show photographs of cells co-cultured with human T cells transduced with anti-MUC1* CAR44 at an MOI of 10. FIGS. 75C, 75G, 75K, 75O show photographs of cells co-cultured with human T cells transduced with anti-MUC1* CAR50 at an MOI of 10. FIGS. 75D, 75H, 75L, 75P show photographs of cells co-cultured with human T cells transduced with anti-MUC1* CAR44 at an MOI of 50, which increases transduction efficiency. FIGS. 75B, 75C, 75D show that both CAR44 and CAR50 transduced T cells recognized MUC1* expressed in these cancer cells, bound to them, induced clustering and killed many cancer cells. FIGS. 75F, 75G, 75H show that neither CAR44 nor CAR50 transduced T cells recognize full-length MUC1 expressed in HCT-MUC1-41TR cancer cells. There is no T cell induced clustering and the number of cancer cells has not decreased. FIGS. 75J, 75K, 75L show that activated MMP9 has cleaved full-length MUC1 to a MUC1* form that is recognized by both CAR44 and CAR50 transduced T cells. There is clearly visible CAR T cell induced clustering and a decrease in the number of cancer cells as they are killed.

FIGS. 75N, 75O, 75P show that activated ADAM17 has either not cleaved MUC1 or cleaved it at a position not recognized by MNC2. Neither huMNC2-CAR44 nor huMNC2-CAR50 transduced T cells recognized these cancer cells.

FIG. 76 shows photographs of a CAR T co-culture assay in which the targeting antibody fragment of the CAR is MNC2 scFv wherein CAR44 has a CD8 transmembrane domain, followed by 41BB-3zeta and CAR50 has a CD4 transmembrane domain, followed by 41BB-3zeta. The target cancer cells are breast cancer T47D cells that were also incubated with conditioned media from cells transfected with MMP2, MMP9 or ADAM17 before co-culture with the MNC2-CAR T cells. In some cases, the conditioned media of the MMP2 and MMP9 expressing cells were also incubated with APMA, which is an activator of these cleavage enzymes. The images shown are an overlay of the 4× bright field image and the fluorescent image of the same showing cancer cells dyed with a red CMTMR lipophilic dye. As can be seen, the MNC2-CAR T cells only bind to and attack the target cancer cells that express the cleaved form, MUC1*.

FIGS. 77A-77I show photographs of cancer cells co-cultured with anti-MUC1* CAR T cells, wherein some of the cancer cells were pre-incubated with activated MMP9 prior to co-culture with the CAR T cells. The cancer cells shown in FIGS. 77A-77C are MUC1 negative colon cancer cell line HCT-116 that have been stably transfected to express MUC1*. The cancer cells shown in FIGS. 77D-77F are MUC1 positive breast cancer cell line T47Ds that express high levels of both MUC1 full-length and MUC1*. The cancer cells shown in FIGS. 77G-77I are MUC1 positive breast cancer cell line T47Ds that were pre-incubated with activated MMP9. The cells shown in FIGS. 77A, 77D and 77G were co-cultured with untransduced human T cells and are the controls. The cells shown in FIGS. 77B, 77E and 77H were co-cultured with human T cells that were transduced with huMNC2-CAR44 at an MOI of 10, wherein MOI stands for multiplicity of infection and the higher the MOI the more CARs are expressed on the T cells. The cells shown in FIGS. 77C, 77F and 77I were co-cultured with human T cells that were transduced with huMNC2-CAR44 at an MOI of 50. As can be seen in the photographs, the CAR44 T cells bind to the target MUC1* positive cancer cells, surrounding and killing them. Comparing the photograph of FIG. S15F with that of FIG. 77I, it can be seen that the cells that were pre-incubated with MMP9 become much more susceptible to CAR T killing when the antibody targeting head of the CAR recognizes MUC1*. It also demonstrates that MUC1 cleaved by MMP9 is recognized by huMNC2scFv.

FIG. 78 shows an xCelligence graph of T47D breast cancer cells in co-culture with either untransduced T cells, as a control, or huMNC2-CAR44 T cells over a 45 hour period. After 18 hours of cancer cell growth, a catalytic sub-unit MMP9 was added to some of the cells. At 25 hours, T cells were added. As can be seen, huMNC2-CAR44 T cell killing is greatly improved when the T47D cells are pre-incubated with cleavage enzyme MMP9. In the xCelligence system, target cancer cells, which are adherent, are plated onto electrode array plates. Adherent cells insulate the electrode and increase the impedance. The number of adherent cancer cells is directly proportional to impedance. T cells are not adherent and do not contribute to impedance. Therefore, increasing impedance reflects growth of cancer cells and decreasing impedance reflects killing of cancer cells.

FIG. 79 shows an xCelligence graph of DU145 prostate cancer cells in co-culture with either untransduced T cells, as a control, or huMNC2-CAR44 T cells over a 45 hour period. After 18 hours of cancer cell growth, a catalytic sub-unit MMP9 was added to some of the cells. At 25 hours, T cells were added. As can be seen, huMNC2-CAR44 T cell killing is not affected by pre-incubation with cleavage enzyme MMP9. DU145 cancer cells express a significantly lower amount of MUC1 which includes the full-length form as well as MUC1*. The lower density of MUC1 full-length does not sterically hinder T cell access to the membrane proximal MUC1*.

FIGS. 80A-80F show photographs of T47D mCherry transfected breast cancer cells co-cultured with either normal human T cells or human T cells transduced with a MUC1* targeting CAR that is GFP positive, green, and wherein the antibody fragment that is the targeting head of the CAR is huMNC2-scFv. FIG. 80A shows breast cancer cells, red, co-cultured with normal human T cells. No T cell induced clustering is evident. FIG. 80B shows breast cancer cells, red, co-cultured with human T cells transduced with huMNC2-CAR18. T cell induced clustering can be seen. FIG. 80C shows the cancer cells co-cultured with huMNC2-CAR19 and T cell induced clustering is seen.

FIG. 80D shows the cancer cells co-cultured with a mixture of huMNC2-CAR44 and CAR49 and T cell induced clustering is seen. FIG. 80E shows the cancer cells co-cultured with a huMNC2-CAR44 and T cell induced clustering is seen. FIG. X1F shows the cancer cells co-cultured with huMNC2-CAR50 and T cell induced clustering is seen.

FIGS. 81A-81D show photographs of human huMNC2-CAR44 T cells injecting granzyme B (yellow) into MUC1* positive and GFP positive (green) DU145 prostate cancer cells. FIG. 81A is a 4× magnified photograph. FIG. 81B is a 20× magnified photograph. FIG. 81C is a 20× magnified photograph. FIG. 81D is a 40× magnified photograph.

FIGS. 82A-82B show the killing effect of huMNC2-CAR44 T cells on T47D MUC1* positive breast cancer cells, wherein the breast cancer cells have been transfected with increasing amounts of additional MUC1*. As can be seen, the killing effect of the huMNC2-CAR44 T cells increases as the amount of target MUC1* expressed on the cells increases. FIG. 82A is a graph of target cell killing as measured by FACS. FIG. 82B is a graph of an ELISA assay in which the supernatant from the huMNC2-CAR44 T cells in co-culture with the T47D cells is probed for the presence of secreted interferon gamma, which is a sign of T cell activation.

FIGS. 83A-83D show the results of FACS analysis of huMNC2-CAR44 T cells after 24 hours of co-culture with MUC1* positive cancer cells. FIG. 83A is a graph of FACS data showing the percentage of T47D cancer cells that were killed by huMNC2-CAR44 T cells (blue bars), compared to untransduced T cells (red bars). The X-axis shows the ratio of T cells to cancer cells. FIG. 83B is a graph of FACS data showing the percentage of K562-MUC1* cancer cells that were killed by huMNC2-CAR44 T cells (blue bars), compared to untransduced T cells (red bars). FIG. 83C shows the FACS scans wherein the T47D breast cancer cells were stained with the dye CMTMR. Sytox blue is a dead cell stain. Dead cancer cells are those in quadrants 2 and 3. FIG. 83D shows the FACS scans wherein the K562-MUC1* cancer cells were stained with the dye CMTMR. Sytox blue is a dead cell stain. Dead cancer cells are those in quadrants 2 and 3.

FIGS. 84A-84H show the cytotoxic effect of huMNC2-CAR44 T cells on MUC1* positive DU145 prostate cancer cells as measured by a variety of assays. FIG. 84A is a fluorescent photograph of untransduced T cells co-cultured with the prostate cancer cells, wherein granzyme B is stained with a red fluorophore. FIG. 84B shows merging of DAPI and granzyme B. FIG. 84C is a fluorescent photograph of huMNC2-CAR44 T cells co-cultured with the prostate cancer cells, wherein granzyme B is stained with a red fluorophore. FIG. 84D shows merging of DAPI and granzyme B. FIG. 84E is a FACS scan for fluorescently labeled granzyme B for untransduced T cells incubated with the cancer cells. FIG. 84F is a FACS scan showing a positive increase in fluorescently labeled granzyme B for huMNC2-CAR44 T cells incubated with the cancer cells. FIG. 84G is a graph of the mean fluorescent intensity. FIG. 84H is an xCELLigence scan tracking the real-time killing of DU145 cancer cells by huMNC2-CAR44 T cells (blue trace) but not by untransduced T cells (green).

FIGS. 85A-85H show the cytotoxic effect of huMNC2-CAR44 T cells on MUC1* positive CAPAN-2 pancreatic cancer cells as measured by a variety of assays. FIG. 85A is a fluorescent photograph of untransduced T cells co-cultured with the pancreatic cancer cells, wherein granzyme B is stained with a red fluorophore. FIG. 85B shows merging of DAPI and granzyme B. FIG. 85C is a fluorescent photograph of huMNC2-CAR44 T cells co-cultured with the pancreatic cancer cells, wherein granzyme B is stained with a red fluorophore. FIG. 85D shows merging of DAPI and granzyme B. FIG. 85E is a FACS scan for fluorescently labeled granzyme B for untransduced T cells incubated with the cancer cells. FIG. 85F is a FACS scan showing a positive increase in fluorescently labeled granzyme B for huMNC2-CAR44 T cells incubated with the cancer cells. FIG. 85G is a graph of the mean fluorescent intensity. FIG. 85H is an xCELLigence scan tracking the real-time killing of CAPAN-2 cancer cells by huMNC2-CAR44 T cells (blue trace) but not by untransduced T cells (green).

FIGS. 86A-86C show xCELLigence scans tracking the real-time killing of MUC1* positive cancer cells, but not MUC1* negative cells, by huMNC2-CAR44 T cells. FIG. 86A shows that huMNC2-CAR44 T cells effectively kill HCT colon cancer cells that have been stably transfected with MUC1*. FIG. 86B shows that huMNC2-CAR44 T cells have almost no effect on HCT-MUC1-41TR, which is a MUC1 negative cancer cell that has been stably transfected with a MUC1 full-length. In this cell line only about 10% of the cells have MUC1 cleaved to MUC1*. FIG. 86C shows that huMNC2-CAR44 T cells have no effect on HCT-116 cells, which is a MUC1 negative colon cancer cell line.

FIGS. 87A-87L show 4× magnification photographs of either untransduced T cells or huMNC2-scFv-CAR44 T cells subjected to either no stimulation, a 1-time bead bearing MUC1* peptide stimulation or a 2-times MUC1* positive cancer cell stimulation. FIGS. 87A-87F show the effect on untransduced T cells. FIGS. 87G-87L show the effect on huMNC2-scFv-CAR44 T cells. FIGS. 87A and 87G received no stimulation. FIGS. 87B and 87H were stimulated twice, 24 hours each stimulation with HCT-MUC1* cancer cells 24 hours prior to photography. FIGS. 87C-87F and FIGS. 871-87L were stimulated once for 24 hours with 1 μm or 4.5 μm beads coated with the PSMGFR MUC1* extra cellular domain peptide 24 hours prior to photography.

FIGS. 88A-88D show FACS analysis of the sub-populations of human T cells transduced with huMNC2-scFv-CAR44 as a result of 1-time stimulation by co-culture with beads bearing MUC1* synthetic peptide or 3-time stimulation by co-culture with HCT-MUC1* cancer cells. FIG. 88A shows FACS scan of huMNC2-scFv-CAR44 transduced human T cells without stimulation. FIG. 88B shows FACS scan of huMNC2-scFv-CAR44 transduced human T cells with 1-time stimulation by co-culture with MUC1* peptide presenting beads. FIG. 88C shows FACS scan of huMNC2-scFv-CAR44 transduced human T cells stimulated 3-times by co-culture with HCT-MUC1* cancer cells. FIG. 88D shows graphical representation of the FACS data. FIGS. 88E-88J show graphs of FACS analysis of T cell activation markers after huMNC2-scFv-CAR44 transduced human T cells were subjected to 1-time MUC1* peptide presenting bead stimulation. FIGS. 88E-88F show FACS of activation marker CD25. FIGS. 88G-88H show FACS of activation marker CD69. FIGS. 88I-88J show FACS of activation marker Granzyme B. FIGS. 88E, 88G, 88I show FACS of huMNC2-scFv-CAR44 transduced human T cells without bead stimulation. FIGS. 88F, 88H, 88J show FACS of huMNC2-scFv-CAR44 transduced human T cells after bead stimulation.

FIGS. 89A-89C show graphs of real time CAR T induced cancer cell killing as measured on an xCELLigence instrument. The figures show the enhanced killing effect of huMNC2-scFv-CAR44 T cells after pre-stimulation by co-culture with MUC1* presenting beads. FIG. 89A shows the enhanced killing effect of peptide bead stimulated huMNC2-CAR44 T cells on SKOV-3 ovarian cancer cells wherein the ratio of T cells to cancer cells was 1:1. FIG. 89B shows the enhanced killing effect of peptide bead stimulated huMNC2-CAR44 T cells on BT-20 triple negative breast cancer cells wherein the ratio of T cells to cancer cells was 1:1. FIG. 89C shows the enhanced killing effect of peptide bead stimulated huMNC2-CAR44 T cells on HCT-MUC1* colon cancer cells wherein the ratio of T cells to cancer cells was 1:1.

FIGS. 90A-90D show graphs of real time cell growth versus cell death as measured on an xCELLigence instrument. Shown is the effect of MUC1* cancer cell stimulation of huMNC2-scFv-CAR44 transduced human T cells on a variety of cancer cells, some of which were previously resistant to CAR T cell killing. FIG. 90A shows an xCELLigence graph of the effect of huMNC2-scFv-CAR44 transduced human T cells that were pre-stimulated by co-culture with HCT-MUC1* cancer cells 24 hours prior to co-culture with the target T47D breast cancer cells. FIG. 90B shows an xCELLigence graph of the effect of huMNC2-scFv-CAR44 transduced human T cells that were pre-stimulated by co-culture with HCT-MUC1* cancer cells 24 hours prior to co-culture with the target BT-20 triple negative breast cancer cells. FIG. 90C shows an xCELLigence graph of the effect of huMNC2-scFv-CAR44 transduced human T cells that were pre-stimulated by co-culture with HCT-MUC1* cancer cells 24 hours prior to co-culture with the target SKOV-3 ovarian cancer cells. FIG. 90D shows an xCELLigence graph of the effect of huMNC2-scFv-CAR44 transduced human T cells that were pre-stimulated by co-culture with HCT-MUC1* cancer cells 24 hours prior to co-culture with the target HCT-MUC1* cancer cells that were effectively killed with or without pre-stimulation.

FIGS. 91A-91Y show fluorescent photographs of mice taken on an IVIS instrument. NSG (NOD/SCID/GAMMA) immune compromised mice that on Day 0 were sub-cutaneously implanted on the flank with 500,000 human MUC1* positive cancer cells that had been stably transfected with Luciferase. Tumors were allowed to engraft. On Day 5 after IVIS measurement and on Day 12, animals were injected with 10 million of either human T cells transduced with huMNC2-scFv-CAR44, untransduced T cells or PBS. 5 million T cells were injected intra-tumor and 5 million T cells were injected into the tail vein. 10 minutes prior to IVIS photographs, mice were injected intraperitoneally (IP) with Luciferin, which fluoresces after cleavage by Luciferase, thus making tumor cells fluoresce. FIGS. 91A, 91E, 91I, 91M, 91Q, 91U show photographs of mice that were treated with huMNC2-scFv-CAR44 T cells that had been pre-stimulated by co-culturing for 24 hours with 4 μm beads to which was attached a synthetic MUC1*, PSMGFR peptide 24 hours prior to administration: Protocol 1. FIGS. 91B, 91F, 91J, 91N, 91R, 91V show photographs of mice that were treated with huMNC2-scFv-CAR44 T cells that had been pre-stimulated by twice co-culturing for 24 hours with MUC1* positive cancer cells 24 hours prior to administration: Protocol 2. FIGS. 91C, 91G, 91K, 91O, 91S, 91W show photographs of mice that were treated with untransduced human T cells. FIGS. 91D, 91H, 91L, 91P, 91T, 91X show photographs of mice that were treated with PBS. FIGS. 91A-91D show IVIS photographs taken on Day 5 before T cell injection. FIGS. 91E-91H show IVIS photographs taken on Day 7. FIGS. 91I-91L show IVIS photographs taken on Day 11. FIGS. 91M-91P show IVIS photographs taken on Day 13. FIGS. 91Q-91T show IVIS photographs taken on Day 18. FIGS. 91U-91V show IVIS photographs taken on Day 21. Animals in untransduced T cell and PBS group had to be sacrificed on Day 20 due to excessive tumor volume. FIGS. 91W-91X show photographs of excised tumors. FIG. 91Y is a color scale relating fluorescence in photons/second to color.

FIGS. 92A-92J show fluorescent photographs of mice taken on an IVIS instrument. NSG (NOD/SCID/GAMMA) immune compromised mice that on Day 0 were sub-cutaneously injected into the flank with 500K human BT-20 cells which are a MUC1* positive triple negative breast cancer cell line. The cancer cells had been stably transfected with Luciferase. Tumors were allowed to engraft. On Day 6 after IVIS measurement, animals were given a one-time injection of 10 million of either human T cells transduced with huMNC2-scFv-CAR44 or untransduced T cells. 5 million T cells were injected intra-tumor and 5 million were injected into the tail vein. 10 minutes prior to IVIS photographs, mice were IP injected with Luciferin, which fluoresces after cleavage by Luciferase, thus making tumor cells fluoresce. FIGS. 92A, 92D, 92G show photographs of mice that were treated with huMNC2-scFv-CAR44 T cells that had been pre-stimulated by co-culturing for 24 hours with 4 μm beads to which was attached a synthetic MUC1*, PSMGFR peptide 24 hours prior to administration: Protocol 1. FIGS. 92B, 92E, 92H show photographs of mice that were treated with huMNC2-scFv-CAR44 T cells that had been pre-stimulated by twice co-culturing for 24 hours with MUC1* positive cancer cells 24 hours prior to administration: Protocol 2. FIGS. 92C, 92F, 92I show photographs of mice that were treated with untransduced human T cells. FIG. 92J is a color scale relating fluorescence in photons/second to color.

FIGS. 93A-93M show fluorescent photographs of mice taken on an IVIS instrument. NSG (NOD/SCID/GAMMA) immune compromised mice that on Day 0 were injected into the intraperitoneal cavity (IP) with 500K human SKOV-3 cells which are a MUC1* positive ovarian cancer cell line. The cancer cells had been stably transfected with Luciferase. Tumors were allowed to engraft. On Day 4, animals were injected into the intraperitoneal space with 10M either human T cells transduced with huMNC2-scFv-CAR44, untransduced T cells or PBS. On Day 11, animals were injected again except that half the cells were injected into the tail vein and the other half was IP injected. Animals were imaged by IVIS on Days 3, 7, 10 and 15. 10 minutes prior to IVIS photographs, mice were IP injected with Luciferin, which fluoresces after cleavage by Luciferase, thus making tumor cells fluoresce. FIGS. 93A, 93D, 93G, and 93J show photographs of mice that were treated with huMNC2-scFv-CAR44 T cells that had been pre-stimulated by co-culturing for 24 hours with 1 μm beads to which was attached a synthetic MUC1*, PSMGFR peptide 24 hours prior to administration. FIGS. 93B, 93E, 93H, and 93K show photographs of mice that were treated with untransduced human T cells. FIGS. 93C, 93F, 93I, and 93L show photographs of mice that were treated with PBS. FIGS. 93A, 93B and 93C are IVIS images taken Day 3 prior to CAR T, T cell or PBS administration. FIGS. 93D, 93E and 93F show IVIS images of animals on Day 7, just four (4) days after treatment. FIGS. 93G, 93H, and 93I show IVIS images of animals on Day 10. FIGS. 93J, 93K, and 93L show IVIS images of animals on Day 15 FIG. 93M is the IVIS color scale relating fluorescence in photons/second to color.

FIGS. 94A-94B are cartoons depicting a steric hindrance problem of MUC1 full-length obstructing access of T cells to the growth factor receptor MUC1*. FIG. 94A is a cartoon showing that late stage cancer cells primarily express cleaved MUC1, such that T cells have easy access to the growth factor receptor. FIG. 94B is a cartoon showing that earlier stage cancer cells express both the MUC1* growth factor receptor and full-length MUC1. Full-length MUC1 is 10-times longer than MUC1* so sterically hinders T cell binding to MUC1*. MMP9 is depicted here as a molecular scissors that, after T cell activation, cuts through full-length protein to make MUC1* more accessible.

FIGS. 95A-95D show Western blots and corresponding FACs analysis of HCT-116 cells which are a MUC1 negative colon cancer cell line, that were then stably transfected with either MUC1* or MUC1 full-length. The single cell clones that are shown are HCT-MUC1-41TR, and HCT-MUC1*. FIG. 95A shows a Western blot of the parent cell line HCT-116, HCT-MUC1-41TR and HCT-MUC1* wherein the gel has been probed with a rabbit polyclonal antibody, SDIX, that only recognizes cleaved MUC1. A visible band between 25 and 35 kDa can be readily seen in Lane 6, loaded with HCT-MUC1*, whereas there is only a faint band in Lanes 4 and 5, showing that only a small amount of MUC1 is cleaved in the HCT-MUC1-41Tr cells. There is no cleaved MUC1 present in the parent cell line HCT-116 loaded into Lanes 2 and 3. FIG. 95B is a Western blot that was probed with a mouse monoclonal antibody VU4H5 that recognizes the tandem repeats of full-length MUC1. As can be seen, only HCT-MUC1-41TR contains full-length MUC1. FIG. 95C shows FACS scans showing that HCT-MUC1* is 95.7% positive for SDIX which only binds to MUC1* and essentially not at all for MUC1 full-length. FIG. 95D shows FACS scans that show that HCT-MUC1-41TR cells are 95% positive for full-length MUC1 and only about 11% positive for the cleaved form, MUC1*.

FIGS. 96A-96E show photographs of an immunofluorescence experiment. HCT-MUC1-41TR cancer cells express full-length MUC1. Notably, the cell line does not naturally cleave MUC1 to MUC1*. Only about 10-15% of the MUC1 is cleaved to a MUC1* form. Here, we show that exposure of MUC1 full-length to MMP9 catalytic domain causes MUC1 cleavage to MUC1* which is recognized by anti-MUC1* antibody MNC2. The amount of binding of MNC2 to the cells is proportional to the amount of MMP9 added to the cells, which shows that MNC2 binds to MUC1 when it is cleaved by MMP9. FIG. 96A is the control and shows HCT-MUC1-41TR cells that have not been incubated with MMP9 but have been stained with MNC2. FIG. 96B shows HCT-MUC1-41TR cells that were incubated with 12.5 ng/mL MMP9 catalytic domain. FIG. 96C shows HCT-MUC1-41TR cells that were incubated with 25 ng/mL MMP9 catalytic domain. FIG. 96D shows HCT-MUC1-41TR cells that were incubated with 50 ng/mL MMP9 catalytic domain. FIG. 96E shows HCT-MUC1-41TR cells that were incubated with 100 ng/mL MMP9 catalytic domain.

FIG. 97 shows a graph of a fluorogenic peptide substrate of MMP9, the OMNIMMP peptide, being cleaved by MMP9 catalytic domain at two concentrations in either PBS, solid trace, or cell culture media, dashed trace.

FIGS. 98A-98F are photographs of Western blots of cell lysates probed with an antibody that recognizes the MMP9 construct that was transfected. A plasmid was constructed then transfected into HEK293T cells, wherein the gene for MMP9 catalytic domain was inserted downstream of either 3 or 4 NFAT response elements. The NFAT pathway was activated by the addition of PMA at 10 ng/mL and lonomycin at either 1 uM or 2 uM, except in control (ctl) cells in lanes 1, 2, 5, 6, 9, 10, 13, and 14. Lysate from cells transfected with the plasmid containing 3 repeats of a NFAT Response element were loaded into lanes 1, 3, 5, 7, 9, 11, 13, and 15. Lysate from cells transfected with the plasmid containing 4 repeats of a NFAT Response element were loaded into lanes 2, 4, 6, 8, 10, 12, 14, and 16. FIG. 98A and FIG. 98C show photographs exposed for 1 minute, whereas FIG. 98B and FIG. 98D show photographs exposed for 5 minutes. To the cell lysates of FIG. 98A and FIG. 98B no protease inhibitor was added. To the cell lysates of FIG. 98C and FIG. 98D a protease inhibitor was added. FIG. 98E shows photograph of Western blot in which MMP9 catalytic domain expressed off repeats of NFAT response element was pulled down from conditioned media of cells whose lysates are shown in FIG. 98A and FIG. 98B, lanes 7 and 8. Pulldown was done using beads to which were coupled an antibody that recognizes a Flag tag that was incorporated at the C-terminus of the MMP9 construct. Lane 1 shows a molecular weight control. Lanes 2, 3, 4 and 5 show MMP9 that was eluted from the anti-Flag tag beads. Lanes 2 and 3 were first elutions and the cells shown in Lanes 4 and 5 were second elutions. Into Lanes 2 and 4 were loaded conditioned media from cells in which the NFAT pathway had been activated with PMA 10 ng/mL and lonomycin at 1 uM. Into Lanes 3 and 5 were loaded conditioned media from cells in which the NFAT pathway had been activated with PMA 10 ng/mL and lonomycin at 2 uM. FIG. 98F is a schematic of the construct.

FIGS. 99A-99C show graphs of a fluorogenic peptide, OMNIMMP peptide, substrate of MMP9 being cleaved by the cell lysate or conditioned media of HEK293T cells that were transfected with a plasmid containing an MMP9 gene downstream from 4 repeats of an NFAT response element. The MMP9 peptide substrate assay shows that activation of the NFAT pathway by PMA/ionomycin caused an MMP9 to be expressed and secreted and that it was active as evidenced by its ability to cleave a peptide substrate. FIG. 99C is a schematic of the construct.

FIGS. 100A-100E show NFAT-induced MMP9 catalytic domain expressed in HEK293T cells wherein the native leader sequence of MMP9 has been replaced by an IgK leader sequence and MMP9 catalytic domain is downstream of 4 repeats of an NFAT response element. FIG. 100A shows photograph of Western blot detecting expression of MMP9 in the cell lysate after activation of the NFAT pathway. FIG. 100B shows photograph of Western blot detecting expression of MMP9 in the conditioned media after activation of the NFAT pathway. FIG. 100C shows graph of MMP9 fluorogenic peptide substrate, OMNIMMP peptide, cleavage by MMP9 catalytic domain expressed and secreted in conditioned media of HEK293T cells wherein the native leader sequence of MMP9 has been replaced by an IgK leader sequence and MMP9 catalytic domain is downstream of 4 repeats of an NFAT response element. FIG. 100D shows graph of MMP9 fluorogenic peptide substrate cleavage by MMP9 catalytic domain expressed and secreted in conditioned media of HEK293T cells wherein the native leader sequence of MMP9 has been replaced by an IgK leader sequence and MMP9 catalytic domain is downstream of 4 repeats of an NFAT response element. FIG. 100E is a schematic of the construct.

FIGS. 101A-101E show MMP9 can be expressed with different leader sequences and also show subsequent activity of each. FIG. 101A shows a Western blot detecting an MMP9 protein in cell lysate wherein the leader sequence upstream of the MMP9 gene is either its native sequence or an IgK sequence. FIG. 101B shows a Western blot detecting MMP9 in conditioned media wherein the leader sequence upstream of the MMP9 gene is its native sequence or an IgK sequence. FIG. 101C shows a graph of an MMP9 peptide substrate cleaved by the expressed MMP9. FIGS. 101D-101E are schematics of the constructs.

FIGS. 102A-102D show three (3) clones 4, 6 and 7 of cells transfected with a plasmid that produces an NFAT inducible MMP9 wherein the NFATc1 promoter sequence is upstream of the MMP9 gene, which in this case is a truncated MMP9 comprising its catalytic domain. Also shown for comparison is a cell transfected with a plasmid that produces an NFAT inducible MMP9 wherein 4 repeats of an NFAT response element sequence are upstream of an MMP9 gene. FIG. 102A shows a Western blot detecting an MMP9 protein in cell lysate. FIG. 102B shows a Western blot detecting MMP9 in the conditioned media. FIGS. 102C-102D are schematics of the constructs.

FIGS. 103A-103D show graphs of an MMP9 peptide substrate cleavage assay. FIG. 103A shows the cleavage activity of MMP9 from the lysate of cells transfected with a plasmid having MMP9 expression driven off of the NFATc1 promoter or off of 4 repeats of an NFAT response element. FIG. 103B shows the cleavage activity of MMP9 from the conditioned media of cells transfected with a plasmid having MMP9 expression driven off of the NFATc1 promoter or off of 4 repeats of an NFAT response element. FIGS. 103C-103D are schematics of the constructs.

FIGS. 104A-104B show the results of the OMNIMMP9 fluorogenic substrate assay that measures activity of MMP9. Conditioned media from human T cells transduced with NFAT-inducible MMP9 alone or in combination with CAR44 were added to the assay and MMP9 substrate cleavage was measured as a function of time. FIG. 104A shows MMP9 activity when human T cells were transduced with both CAR44 and an NFAT-inducible MMP9 after the cells were activated by co-culturing with HCT-MUC1* cancer cells. The trace that does not show increased substrate cleavage as a function of time is the conditioned media from cells that were not activated. FIG. 104B shows MMP9 activity when human T cells were transduced with just an NFAT-inducible MMP9 after the cells were activated by co-culturing with beads coated with anti-CD3 and anti-CD28 which are known to activate T cells. The trace that does not show increased substrate cleavage as a function of time is the conditioned media from cells that were not activated.

FIGS. 105A-105E show photographs of Western blots of human T cells transduced with either CAR44 alone, NFAT-inducible MMP9 alone or transduced with both CAR44 and NFAT-inducible MMP9, wherein the resultant T cells are either not activated, chemically activated by PMA/Ionomycin, activated by co-culturing with beads presenting synthetic MUC1* peptide or co-culturing with MUC1* positive cancer cells. Western blot was probed with an anti-Flag tag also known as DYK tag antibody. Catalytic domain of MMP9 runs with an apparent molecular weight of about 40 kDa. FIGS. 105A-105D show photographs of Western blots of cleared cell lysates. FIG. 105A has Lanes 1-7 loaded with lysates of: Lane 1: T cells transduced with CAR44 and not activated; Lane 2: T cells transduced with CAR44 and activated with beads presenting synthetic MUC1* extra cellular domain peptide; Lane 3: T cells transduced with CAR44 and activated by co-culture with HCT-MUC1* cancer cells; Lane 4: T cells transduced with CAR44 and NFAT-inducible MMP9 but not activated; Lane 5: T cells transduced with CAR44 and NFAT-inducible MMP9 and activated with beads presenting synthetic MUC1* extra cellular domain peptide; Lane 6: T cells transduced with CAR44 and NFAT-inducible MMP9 and activated by co-culture with HCT-MUC1* cancer cells; Lane 7: an irrelevant protein also bearing the Flag DYK tag. Results show that T cells transduced with NFAT-inducible MMP9 only express MMP9 when they are activated by PMA/Ionomycin, MUC1* beads or MUC1* positive cancer cells. T cells transduced with both CAR44 and NFAT-inducible MMP9 only express MMP9 when the T cells are activated by stimulation with MUC1* beads or with MUC1* positive cancer cells. FIG. 105B has Lanes 1-7 loaded with lysates of: Lane 1: T cells transduced with CAR44 and not activated; Lane 2: T cells transduced with CAR44 and activated with beads presenting anti-CD3 and anti-CD28 antibodies that are known to activate T cells; Lane 3: T cells transduced with CAR44 and activated by co-culture with PMA/Ionomycin; Lane 4: T cells transduced with NFAT-inducible MMP9 but not activated; Lane 5: T cells transduced with NFAT-inducible MMP9 and activated with beads presenting anti-CD3 and anti-CD28 antibodies; Lane 6: T cells transduced with NFAT-inducible MMP9 and activated by PMA/Ionomycin; Lane 7: an irrelevant protein also bearing the Flag DYK tag. FIGS. 105C and 105D are darker exposures of the same Western blots shown in FIGS. 105A and 105B, respectively. FIG. 105E is a photograph of a Western blot of cell supernatants of cells transduced as follows: Lane 1: T cells transduced with CAR44 and not activated; Lane 2: T cells transduced with CAR44 and activated with beads presenting anti-CD3 and anti-CD28 antibodies that are known to activate T cells; Lane 3: T cells transduced with CAR44 and activated by co-culture with PMA/Ionomycin; Lane 4: T cells transduced with NFAT-inducible MMP9 but not activated; Lane 5: T cells transduced with NFAT-inducible MMP9 and activated with beads presenting anti-CD3 and anti-CD28 antibodies; Lane 6: T cells transduced with NFAT-inducible MMP9 and activated by PMA/Ionomycin; Lane 7: an irrelevant protein also bearing the Flag DYK tag. Results show that T cells transduced with NFAT-inducible MMP9 express MMP9 when they are activated. T cells transduced with both CAR44 and NFAT-inducible MMP9 are specifically activated when they are co-cultured with beads or cells presenting or expressing MUC1* (FIG. 105A Lane 5 and Lane 6).

FIGS. 106A-106B show a cartoon of a series of “long-arm” CARs that were generated to overcome the steric hindrance caused by full-length MUC1. FIG. 106A shows a cartoon of the CARs with longer linker region between the cell membrane and the antibody scfv. FIG. 106B shows a cartoon of how they overcome steric hindrance of MUC1 full-length.

FIGS. 107A-107B show xCelligence graphs of MUC1 positive breast cancer T47D cells in co-culture with either untransduced T cells, as a control, or several different long-arm CAR T cells, wherein the length and sequence of the linker between the antibody scFv and the transmembrane domain is varied as indicated. FIG. 107A shows impedance as a function of time for the various CAR T cells that were tested. FIG. 107B shows the same data but wherein the slope of the trace is graphed as a function of time.

FIGS. 108A-108P show photographs of a cell binding assay in which cells were transduced with CARs having variable length linker regions between the antibody fragment and the transmembrane domain. The CAR-transduced cells carry a GFP fluorescent maker so are green. MUC1* positive cancer cells that have been stained red with CMTMR dye are then added to the CAR expressing cells. The degree to which the CARs are able to recognize their target on cancer cells is reflected by the amount of yellow (green plus red). FIG. 108A is the control, untransduced cells. FIG. 108B cells were transduced with CAR44 where the linker region is derived from CD8 extra cellular domain. FIG. 108C shows a CAR with a linker that is a portion of an antibody Fc region. FIG. 108D shows a CAR with a linker that is a portion of an antibody Fc region, minus its hinge region. FIG. 108E shows a CAR with a linker that is a 4-repeat flexible linker sequence. FIG. 108F shows a CAR with a linker that is a portion of an IgD antibody. FIG. 108G shows a CAR with a linker that is a portion of an IgD antibody plus an Fc region. FIG. 108H shows a CAR with a linker that is a portion of an IgD antibody plus an Fc region devoid of its hinge region. FIGS. 1081-108M show photographs of CAR expressing cells after incubation with MUC1* expressing cancer cells, after sufficient wash steps.

Table 1 shows details of many of the anti-MUC1* CARs that were generated and tested. For each construct shown, a number assigned to that CAR, promoter used, signal peptide, antibody species, sequences of scFv, hinge region, transmembrane domain, and signaling motifs used in each CAR, length of the insert in number of base pairs, its molecular weight and the length of the construct are displayed.

Table 2 shows cytokine release data for some of the CARs after transduction into human T cells and co-cultured with a variety of cancer cells.

### DETAILED DESCRIPTION OF THE PREFERRED EMBODIMENTS

In the present application, “a” and “an” are used to refer to both single and a plurality of objects.

As used herein, occasionally, in short hand, a polypeptide is indicated as being “transduced or transfected” into a cell. In these occurrences, it is understood that the nucleic acid encoding the polypeptide sequence is transduced or transfected into the cell, as it is an impossibility that a polypeptide could be transduced or transfected into a cell.

As used herein, occasionally when referring to number of cells injected into an animal or otherwise contextually wherein the number of cells is referred to, “M” refers to millions, and “K” refers to thousands.

As used herein, interchangeable designations for various monoclonal antibodies are used, such as, “MN-C2”, which is interchangeable with “C2”, “Min-C2” and “MNC2”; “MN-E6”, which is interchangeable with “E6”, “Min-E6” and “MNE6”; “MN-C3”, which is interchangeable with “C3”, “Min-C3” and “MNC3”; and “MN-C8”, which is interchangeable with “C8”, “Min-C8” and “MNC8”.

As used herein, “h” or “hu” placed before an antibody construct is short-hand for humanized.

As used herein, the term “antibody-like” means a molecule that may be engineered such that it contains portions of antibodies but is not an antibody that would naturally occur in nature. Examples include but are not limited to CAR (chimeric antigen receptor) T cell technology and the Ylanthia® technology. The CAR technology uses an antibody epitope fused to a portion of a T cell so that the body's immune system is directed to attack a specific target protein or cell. The Ylanthia® technology consists of an “antibody-like” library that is a collection of synthetic human Fabs that are then screened for binding to peptide epitopes from target proteins. The selected Fab regions can then be engineered into a scaffold or framework so that they resemble antibodies.

As used herein, “PSMGFR” is abbreviation for Primary Sequence of the MUC1 Growth Factor Receptor which is identified by SEQ ID NO:2, and thus is not to be confused with a six amino acid sequence. “PSMGFR peptide” or “PSMGFR region” refers to a peptide or region that incorporates the Primary Sequence of the MUC1 Growth Factor Receptor (SEQ ID NO:2).

As used herein, the “MUC1*” extra cellular domain is defined primarily by the PSMGFR sequence (GTINVHDVETQFNQYKTEAASRYNLTISDVSVSDVPFPFSAQSGA (SEQ ID NO:2)). Because the exact site of MUC1 cleavage depends on the enzyme that clips it, and that the cleavage enzyme varies depending on cell type, tissue type or the time in the evolution of the cell, the exact sequence of the MUC1* extra cellular domain may vary at the N-terminus.

Other clipped amino acid sequences may include SNIKFRPGSVVVQLTLAFREGTINVHDVETQFNQYKTEAASRY (SEQ ID NO:620); or SVVVQLTLAFREGTINVHDVETQFNQYKTEAASRY (SEQ ID NO:621).

As used herein, the term “PSMGFR” is an acronym for Primary Sequence of MUC1 Growth Factor Receptor as set forth as GTINVHDVETQFNQYKTEAASRYNLTISDVSVSDVPFPFSAQSGA (SEQ ID NO:2). In this regard, the “N-number” as in “N-10 PSMGFR”, “N-15 PSMGFR”, or “N-20 PSMGFR” refers to the number of amino acid residues that have been deleted at the N-terminal end of PSMGFR. Likewise “C-number” as in “C-10 PSMGFR”, “C-15 PSMGFR”, or “C-20 PSMGFR” refers to the number of amino acid residues that have been deleted at the C-terminal end of PSMGFR.

As used herein, the “extracellular domain of MUC1*” refers to the extracellular portion of a MUC1 protein that is devoid of the tandem repeat domain. In most cases, MUC1* is a cleavage product wherein the MUC1* portion consists of a short extracellular domain devoid of tandem repeats, a transmembrane domain and a cytoplasmic tail. The precise location of cleavage of MUC1 is not known perhaps because it appears that it can be cleaved by more than one enzyme. The extracellular domain of MUC1* will include most of the PSMGFR sequence but may have an additional 10-20 N-terminal amino acids.

As used herein “sequence identity” means homology in sequence of a particular polypeptide or nucleic acid to a reference sequence of nucleic acid or amino acid such that the function of the homologous peptide is the same as the reference peptide or nucleic acid. Such homology can be so close with the reference peptide such that at times the two sequences may be 90%, 95% or 98% identical yet possess the same function in binding or other biological activities.

As used herein, “MUC1 positive” cell refers to a cell that expresses a gene for MUC1, MUC1-Y or MUC1-Z or other MUC1 variant.

As used herein, “MUC1 negative” cell refers to a cell that does not express a gene for MUC1.

As used herein, “MUC1* positive” cell refers to a cell that expresses a gene for MUC1, wherein that gene's expressed protein is a transmembrane protein that is devoid of tandem repeats, which may be a consequence of post-translational modification, cleavage, alternative splicing, or transfecting or transducing a cell with a MUC1 protein that is devoid of tandem repeats.

As used herein, “MUC1* negative” cell refers to a cell that may or may not express a gene for MUC1 but does not express a MUC1 transmembrane protein that is devoid of tandem repeats.

As used herein, “MUC1 positive” cancer cell refers to a cancer cell that overexpresses the gene for MUC1, expresses MUC1 in an aberrant pattern, wherein its expression is not restricted to the apical border and/or expresses a MUC1 that is devoid of tandem repeats.

As used herein, “MUC1 negative” cancer cell refers to a cancer cell that may or may not express a gene for MUC1 but does not overexpress MUC1 or does not overexpress a MUC1 transmembrane protein that is devoid of tandem repeats.

As used herein, “MUC1* positive” cancer cell refers to a cancer cell that overexpresses a MUC1 transmembrane protein that is devoid of tandem repeats.

As used herein, “MUC1* negative” cancer cell refers to a cancer cell that may or may not express a gene for MUC1 but does not overexpress a MUC1 transmembrane protein that is devoid of tandem repeats.

MUC1* Antibodies (Anti-PSMGFR) for Treatment or Prevention of Cancers

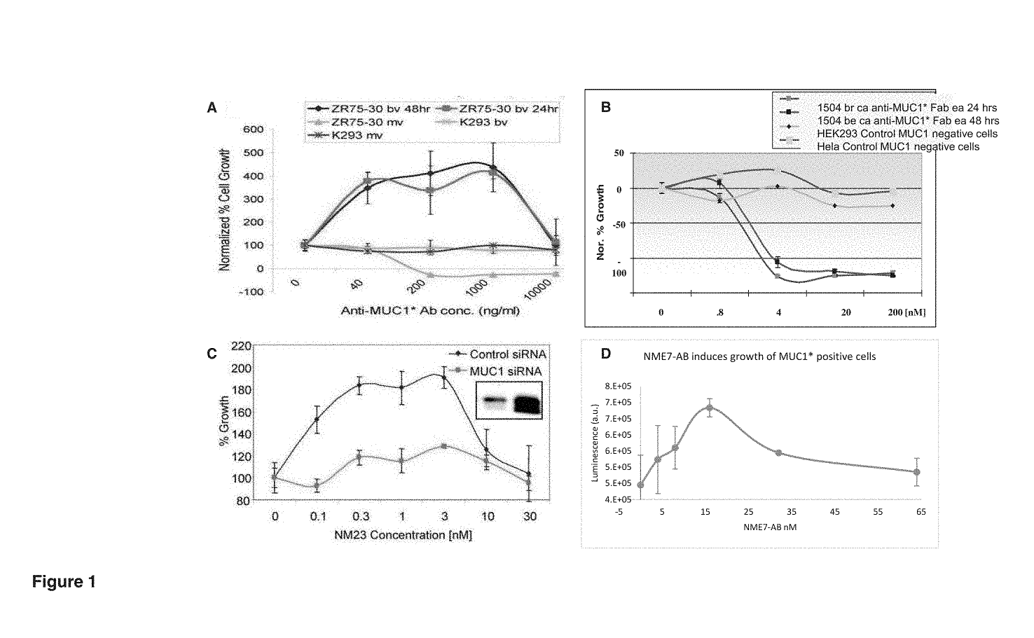

We discovered that a cleaved form of the MUC1 (SEQ ID NO:1) transmembrane protein is a growth factor receptor that drives the growth of over 75% of all human cancers. The cleaved form of MUC1, which we called MUC1* (pronounced muk 1 star), is a powerful growth factor receptor. Enzymatic cleavage releases the bulk of the MUC1 extracellular domain. It is the remaining portion comprising a truncated extracellular domain, transmembrane domain and cytoplasmic tail that is called MUC1*. Cleavage and release of the bulk of the extracellular domain of MUC1 unmasks a binding site for activating ligands dimeric NME1, NME6, NME8, NME7-AB, NME7-X1 or NME7. Cell growth assays show that it is ligand-induced dimerization of the MUC1* extracellular domain that promotes growth (FIGS. 1A-1D). MUC1* positive cells treated with either bivalent ‘bv’ anti-MUC1* antibody, monovalent ‘my’ or Fab, NM23-H1 dimers or NME7-AB. Bivalent anti-MUC1* antibodies stimulate growth of cancer cells whereas the monovalent Fab inhibits growth. Classic bell-shaped curve indicates ligand induced dimerization stimulates growth. Dimeric NM23-H1, aka NME1, stimulates growth of MUC1* positive cancer cells but siRNA to suppress MUC1 expression eliminate its effect (FIG. 1C). NME7-AB also stimulates the growth of MUC1* positive cells (FIG. 1D).

MUC1* is an excellent target for cancer drugs as it is aberrantly expressed on over 75% of all cancers and is likely overexpressed on an even higher percentage of metastatic cancers. After MUC1 cleavage, most of its extracellular domain is shed from the cell surface. The remaining portion has a truncated extracellular domain that at least comprises the primary growth factor receptor sequence, PSMGFR (SEQ ID NO:2). Antibodies that bind to the PSMGFR sequence and especially those that competitively inhibit the binding of activating ligands such as NME proteins, including NME1, NME6, NME8, NME7AB, NME7-X1 and NME7, are ideal therapeutics and can be used to treat or prevent MUC1 positive or MUC1* positive cancers, as stand-alone antibodies, antibody fragments or variable region fragments thereof incorporated into bispecific antibodies, or chimeric antigen receptors also called CARs, which are then transfected or transduced into immune cells, then administered to a patient.

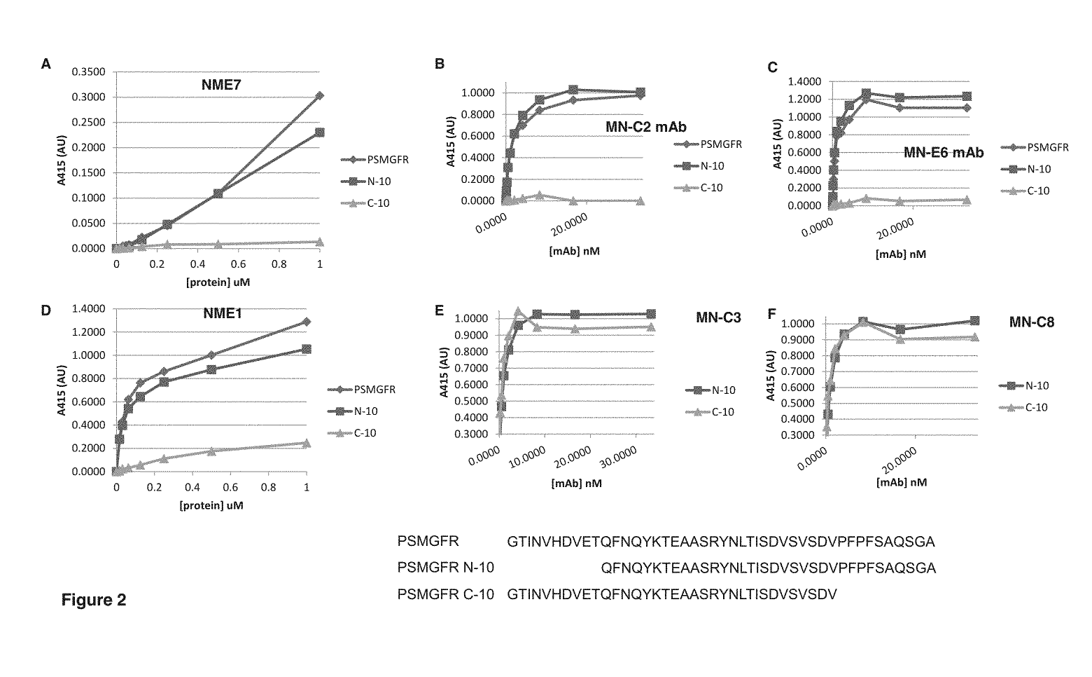

Therapeutic anti-MUC1* antibodies can be monoclonal, polyclonal, antibody mimics, engineered antibody-like molecules, full antibodies or antibody fragments. Examples of antibody fragments include but are not limited to Fabs, scFv, and scFv-Fc. Human or humanized antibodies are preferred for use in the treatment or prevention of cancers. In any of these antibody-like molecules, mutations can be introduced to prevent or minimize dimer formation. Anti-MUC1* antibodies that are monovalent or bispecific are preferred because MUC1* function is activated by ligand induced dimerization. Typical binding assays show that NME1 and NME7-AB bind to the PSMGFR peptide portion of MUC1* (FIGS. 2A, 2D). Further, they show that these activating growth factors bind to the membrane proximal portion of MUC1*, as they do not bind to the PSMGFR peptide if the 10 C-terminal amino acids are missing. Similarly, anti-MUC1* antibodies MN-C2 and MN-E6 bind to the PSMGFR peptide if an only if the 10 C-terminal amino acids are present (FIGS. 2B, 2C). Antibodies MN-C3 and MN-C8 bind to epitopes that are different from MN-C2 and MN-E6, as they do not depend on the presence of the 10 C-terminal amino acids of the PSMGFR peptide (FIGS. 2E, 2F). Antibodies MN-C2, MN-E6, MN-C3 or MN-C8, or fragments derived from them, can be administered to a patient for the treatment or prevention of cancers, as stand-alone antibodies or incorporated into bispecific antibodies, BiTEs or chimeric antigen receptors also called CARs that have been transduced into immune cells. MNC2 and MNE6 and other anti-MUC1* antibodies that competitively inhibit the binding of NME1 and NME7-AB are preferred for use as stand alone antibody therapeutics.

Therapeutic anti-MUC1* antibodies for use as a stand alone antibody therapeutic or for integration into a BiTE or a CAR can be selected based on specific criteria. The parent antibody can be generated using typical methods for generating monoclonal antibodies in animals. Alternatively, they can be selected by screening antibody and antibody fragment libraries for their ability to bind to a MUC1* peptide, which can be the PSMGFR peptide (SEQ ID NO:2), SNIKFRPGSVVVQLTLAFREGTINVHDVETQFNQYKTEAASRY (SEQ ID NO:620); or SVVVQLTLAFREGTINVHDVETQFNQYKTEAASRY (SEQ ID NO:621).

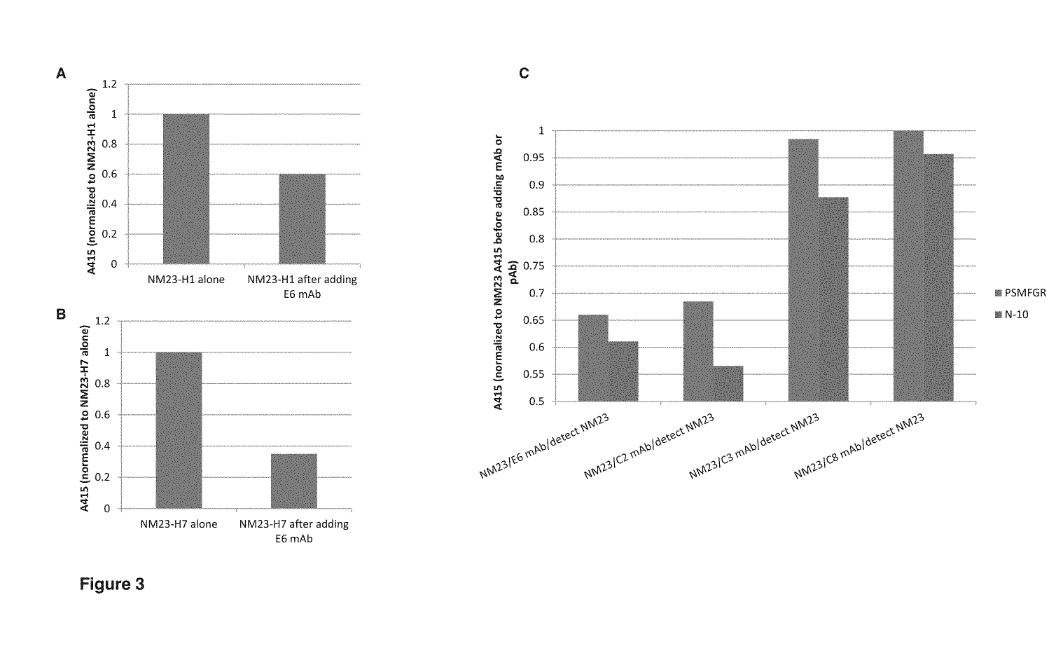

Resultant antibodies or antibody fragments generated or selected in this way can then be further selected by passing additional screens. For example, antibodies or antibody fragments become more preferred based on their ability to bind to MUC1* positive cancer cells or tissues but not to MUC1 negative cancer cells or to normal tissues. Further, anti-MUC1* antibodies or antibody fragments may be de-selected as anti-cancer therapeutics if they bind to stem or progenitor cells. Anti-MUC1* antibodies or antibody fragments become more preferred if they have the ability to competitively inhibit the binding of activating ligands to MUC1*. FIGS. 3A-3C shows that MN-E6 and MN-C2 competitively inhibit the binding of activating ligands NME1 and NME7 to MUC1*.

A process for selecting anti-MUC1* antibodies for use in treating a patient diagnosed with a MUC1 positive cancer, at risk of developing a MUC1 positive cancer or suspected of having a MUC1 positive cancer comprises one or more of the following steps of selecting antibodies or antibody fragments that 1) bind to the PSMGFR peptide; 2) bind to the N-10 PSMGFR peptide; 3) bind to cancer cells; 4) do not bind to stem or progenitor cells; and 5) competitively inhibited the binding of dimeric NME1 or NME7-AB to the PSMGFR peptide. For example, FIGS. 3A-3C show that monoclonals MN-E6 and MN-C2 satisfy all five criteria, while monoclonals MN-C3 and MN-C8 do not competitively inhibit the binding of activating ligands NME1 and NME7 (FIG. 3C). However, antibodies or antibody fragments derived from MN-C3 and MN-C8 are equally potent as anti-cancer agents when integrated into a BiTE or a CAR as in these methods, the killing effect of the immune cells is more important than the ability to inhibit the binding of activating ligands. In addition, toxic agents conjugated to MN-E6, MN-C2, MN-C3 or MN-C8 are potent anti-cancer therapeutics. Recall that the MUC1* growth factor receptor is activated by ligand-induced dimerization of its extracellular domain. Therefore, the ideal antibody therapeutic should not dimerize the MUC1* extracellular domain. Preferably, suitable antibodies in this regard include monovalent antibodies such as those generated in lamas and camels, Fabs, scFv's, single domain antibodies (sdAb), scFv-Fc as long as the Fc portion is constructed such that it does not homo-dimerize.

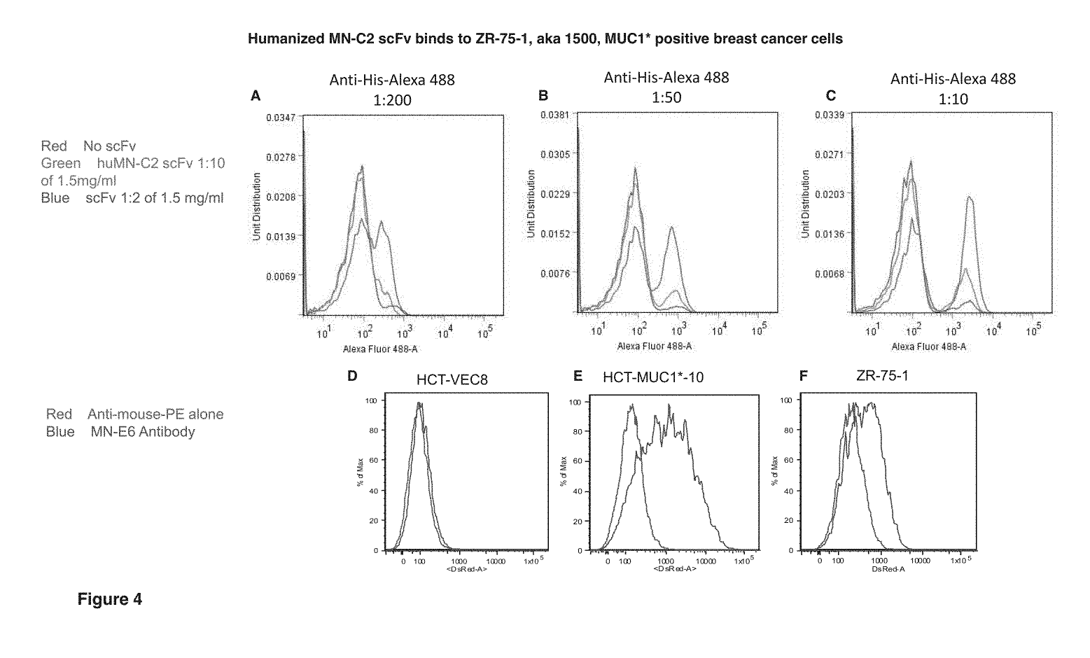

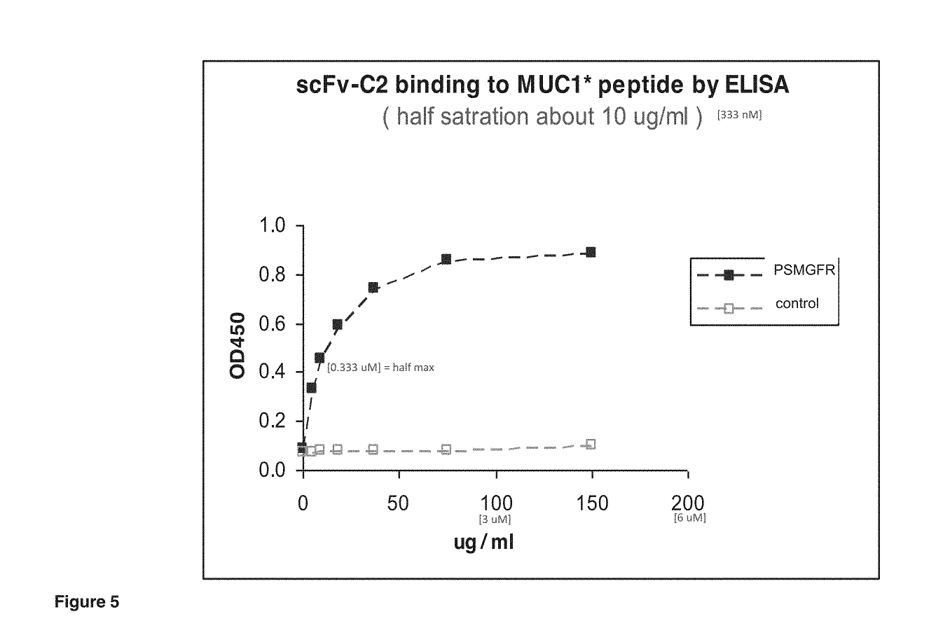

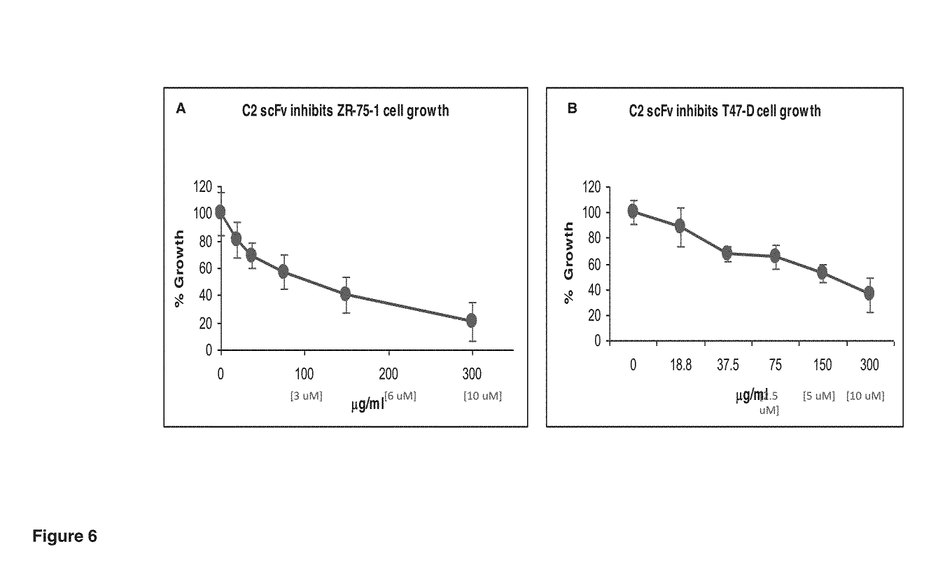

FACS scans show that anti-MUC1* antibodies MN-C2 and MN-E6 specifically bind to MUC1* positive solid tumor cancer cells and MUC1* transfected cells but not MUC1* negative or MUC1 negative cells. MNC3 and MNC8 bind to blood progenitor cells as well as to blood cancer cells, since these diseases are characterized by the inability of blood progenitor cells to terminally differentiate. Therefore, MNC3 and MNC8 are preferred for the treatment of blood cancers, as stand alone therapeutics, BiTEs or CAR T therapeutics. In one example, a humanized MN-C2 scFv is shown to bind to ZR-75-1, aka 1500, MUC1* positive breast cancer cells (FIGS. 4A-4C). MN-E6 was shown to bind to MUC1 negative HCT-116 colon cancer cells if an only if they were transfected with MUC1*. MN-E6 also bound to MUC1* positive cancer cells such as ZR-75-1, aka 1500, MUC1* positive breast cancer cells (FIGS. 4D-4F). Binding assays such as ELISAs, immunofluorescence, and the like all confirm that MN-C2 and MN-E6 bind to the PSMGFR peptide and to live MUC1 positive cancer cells. Humanized anti-MUC1* antibodies are selected based on their ability to also bind to the PSMGFR peptide or to MUC1 positive cancer cells. FIG. 5 shows that humanized MN-C2 scFv binds with high affinity to the MUC1* peptide PSMGFR with an EC-50 of about 333 nM. Humanized MN-C2 scFv, like Fabs, potently inhibits the growth of MUC1* positive cancer cells as is shown in one example in FIGS. 6A, 6B.

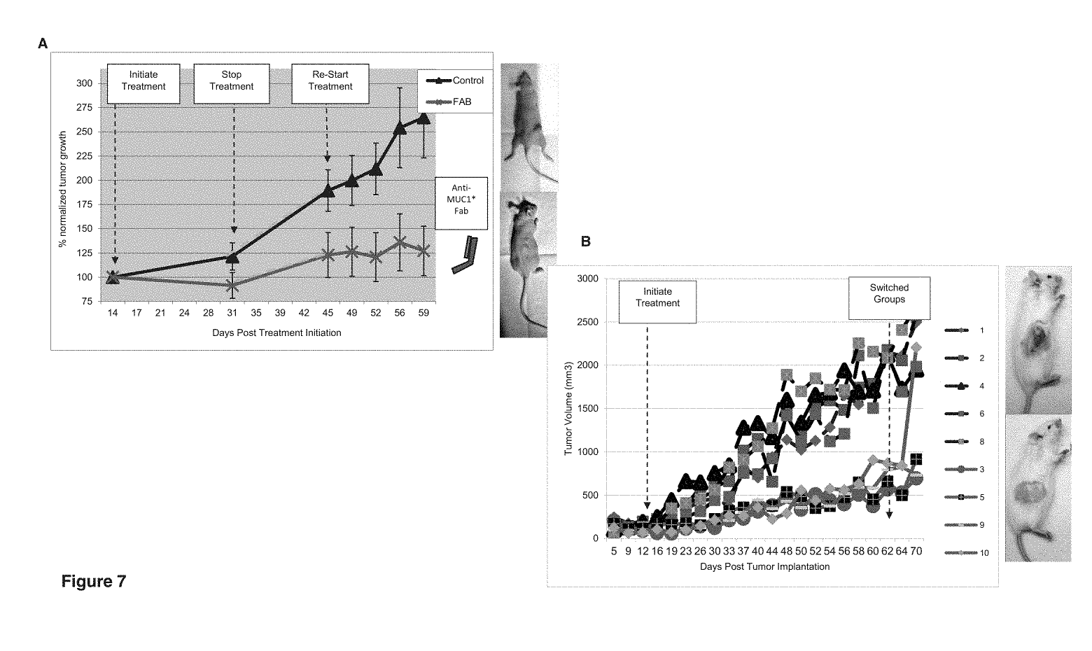

The Fabs of MN-E6 and MN-C2 or the comparable single chain variable regions derived from them potently inhibit the growth of MUC1* positive cancers in vitro and in vivo. In several examples, the Fabs of Anti-MUC1* antibodies inhibited the growth of human MUC1* positive cancers in vivo. In one case, immune-compromised mice were implanted with human breast tumors then treated with MN-E6 Fab after tumor engraftment. FIG. 7A shows that MN-E6 Fab potently inhibited the growth of MUC1* positive breast cancers. Female nu/nu mice implanted with 90-day estrogen pellets were implanted with 6 million T47D human breast cancer cells that had been mixed 50/50 with Matrigel. Mice bearing tumors that were at least 150 mm\hat{ }3 and had three successive increases in tumor volume were selected for treatment. Animals were injected sub-cutaneously twice per week with 80 mg/kg MN-E6 Fab and an equal number of mice fitting the same selection criteria were injected with vehicle alone (FIG. 7A).

In another aspect, MN-E6 was shown to halt the growth of prostate cancer. FIG. 7B shows that MN-E6 Fab potently inhibited the growth of MUC1* positive prostate cancers. Male NOD/SCID mice were implanted with 6 million DU-145 human prostate cancer cells that had been mixed 50/50 with Matrigel. Mice bearing tumors that were at least 150 mm\hat{ }3 and had three successive increases in tumor volume were selected for treatment. Animals were injected sub-cutaneously every 48 hours with 160 mg/kg MN-E6 Fab and an equal number of mice fitting the same selection criteria were injected with vehicle alone (FIG. 7B). Tumors were measured independently by two researchers twice per week and recorded. Statistics were blindly calculated by independent statistician, giving a P value of 0.0001 for each. Anti-MUC1* Fab inhibited breast cancer growth and prostate cancer growth. Treatment had no effect on weight, bone marrow cell type or number. The MN-E6 Fab effectively inhibited the growth of the tumors, while the control group's tumors continued to grow until sacrifice. No adverse effects of treatment were observed or detected.

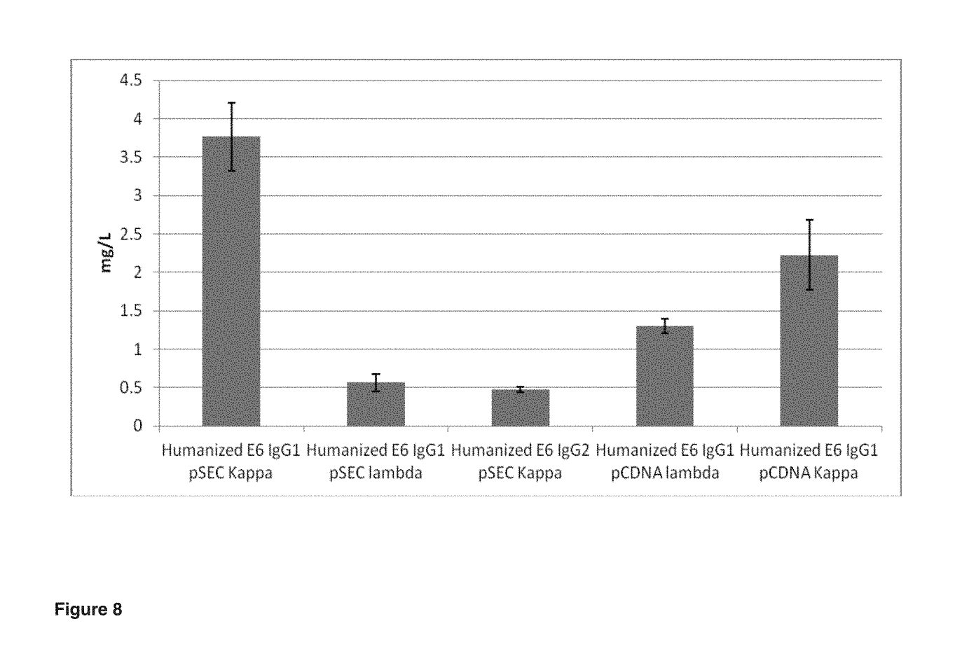

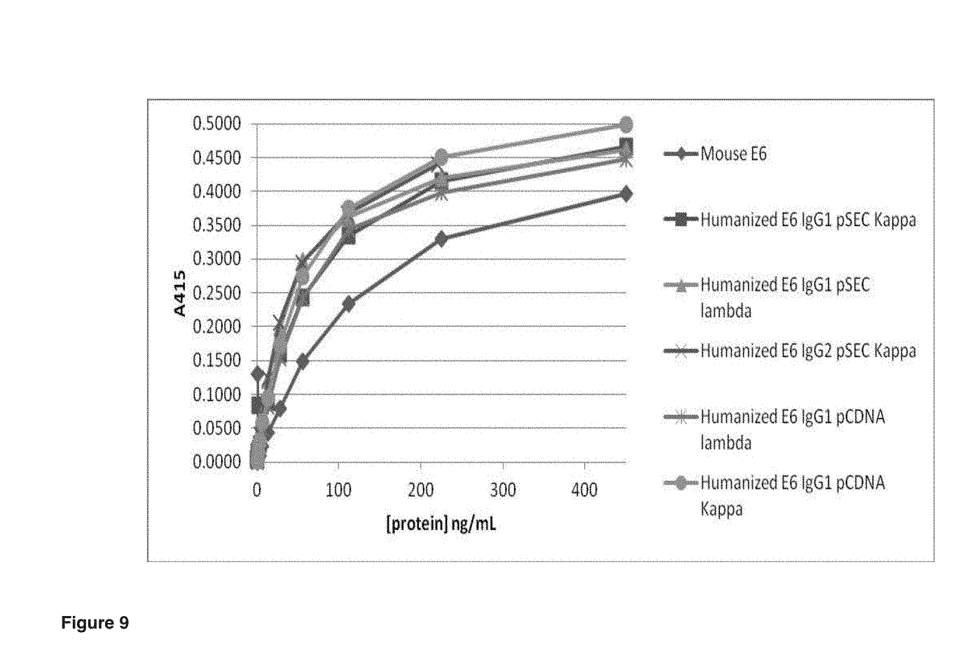

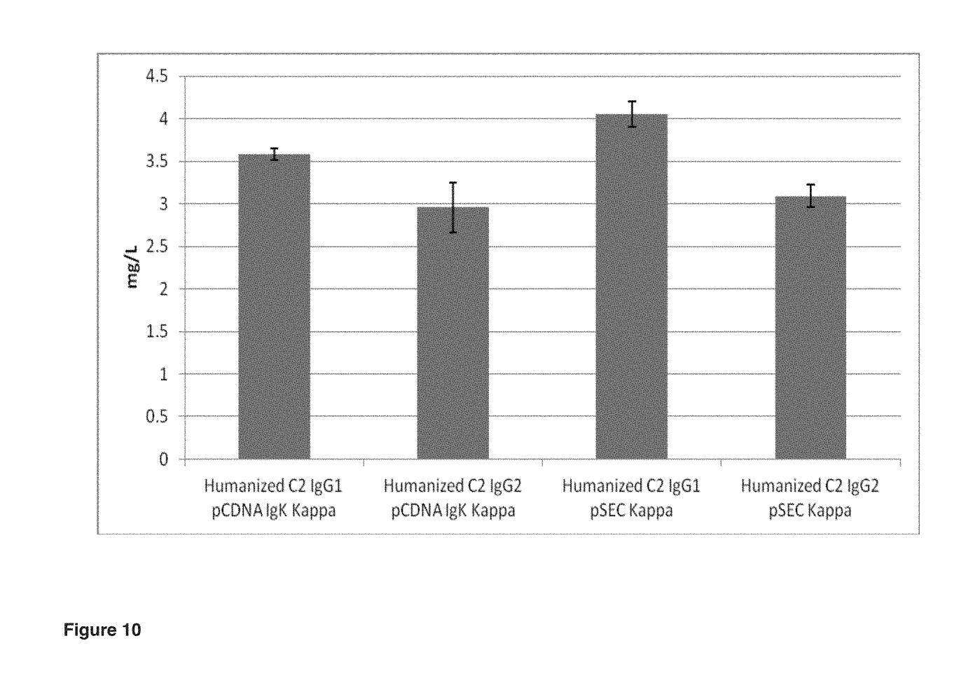

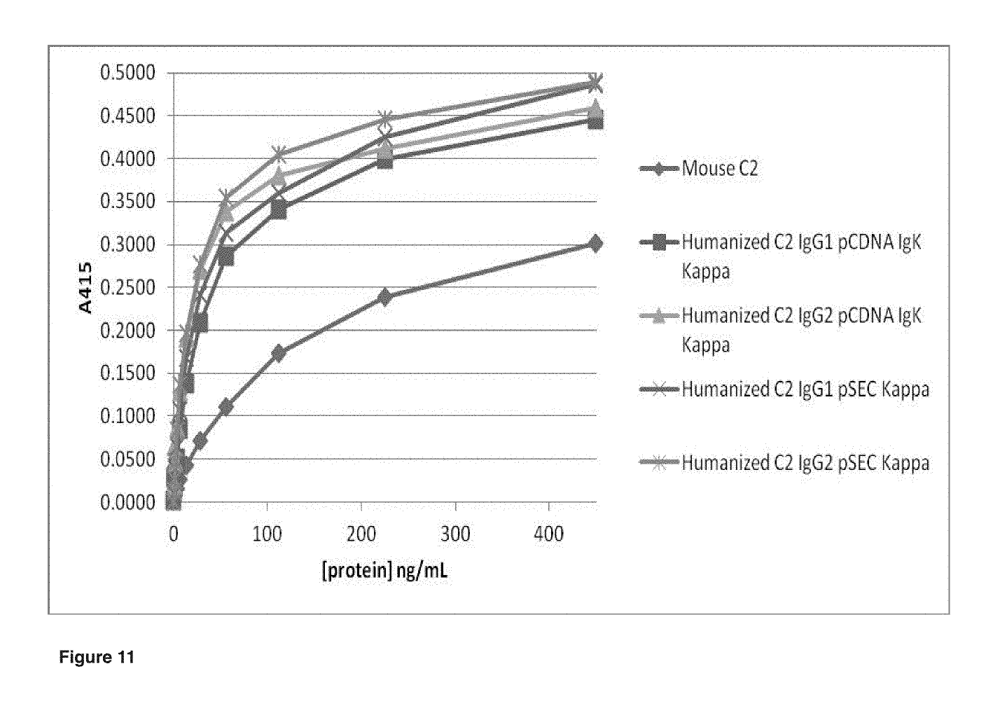

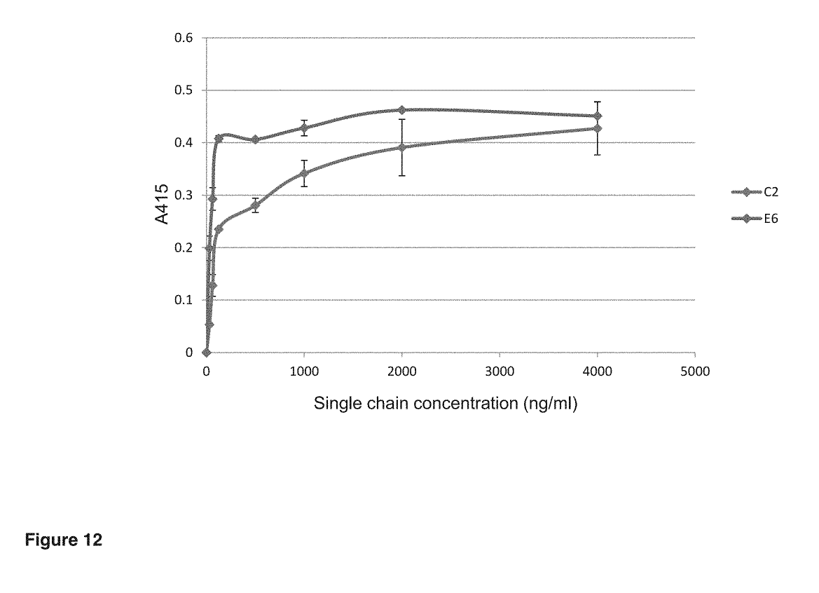

Recombinant forms of MN-E6 and MINERVA-C2 were constructed that like the Fab are monomeric. In this case, MN-E6 was humanized and MINERVA-C2 was humanized. There are a number of methods known to those skilled in the art for humanizing antibodies. In addition to humanizing, libraries of human antibodies can be screened to identify other fully human antibodies that bind to the PSMGFR. FIG. 8 is a graph of an ELISA assay showing differing levels of expression of humanized MN-E6 anti-MUC1* antibody depending on whether the light chain was kappa or lambda and whether the variable portion was fused to a human IgG1 or IgG2. FIG. 9 is a graph of an ELISA assay comparing the binding of the parent mouse MN-E6 antibody to the humanized versions of the MN-E6 antibody to a surface presenting the PSMGFR peptide derived from the MUC1* extracellular domain. FIG. 10 is a graph of an ELISA assay showing differing levels of expression of humanized MN-C2 anti-MUC1* antibody depending on whether the light chain was kappa or lambda and whether the variable portion was fused to a human IgG1 or IgG2. FIG. 11 is a graph of an ELISA assay comparing the binding of the parent mouse MN-C2 antibody to the humanized versions of the MN-C2 antibody to a surface presenting the PSMGFR peptide derived from the MUC1* extracellular domain. FIG. 12 is a graph of an ELISA assay showing binding of humanized single chain (scFv) MN-C2 and MN-E6 antibodies binding to a surface presenting the PSMGFR peptide derived from the MUC1* extracellular domain.

A single chain of the humanized MN-E6 variable region, called an scFv, was genetically engineered such that it was connected to the Fc portion of the antibody (SEQ ID NO:256 and 257). Fc regions impart certain benefits to antibody fragments for use as therapeutics. The Fc portion of an antibody recruits complement, which in general means it can recruit other aspects of the immune system and thus amplify the anti-tumor response beyond just inhibiting the target. The addition of the Fc portion also increases the half-life of the antibody fragment (Czajkowsky D M, Hu J, Shao Z and Pleass R J. (2012) Fc-fusion proteins: new developments and future perspectives. EMBO Mol Med. 4(10):1015-1028).

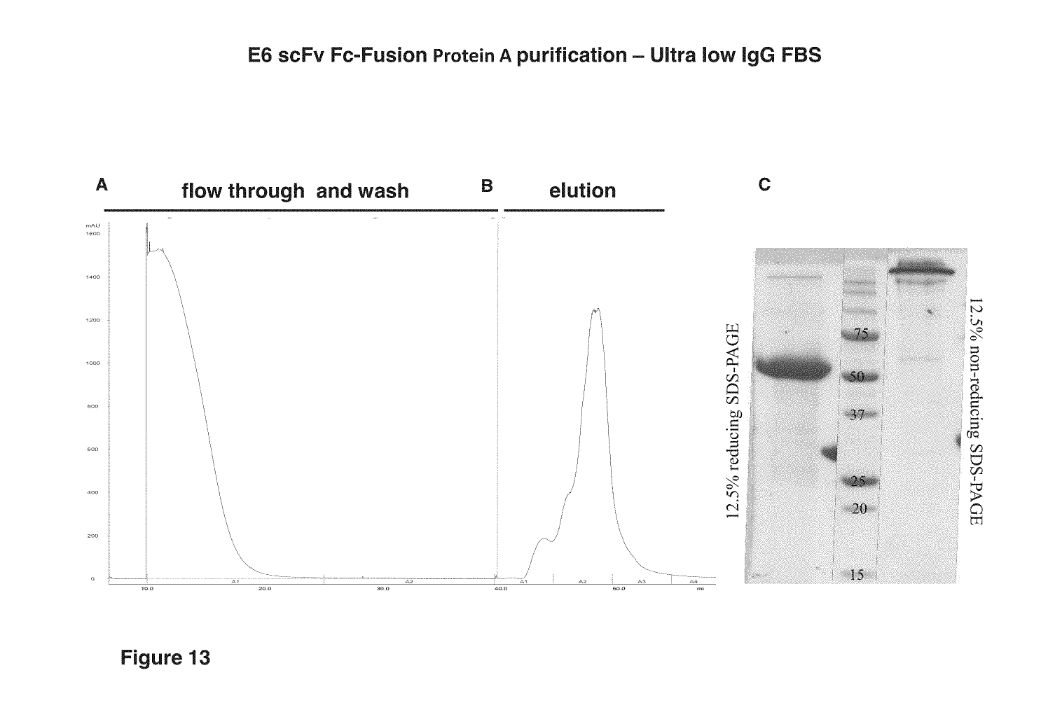

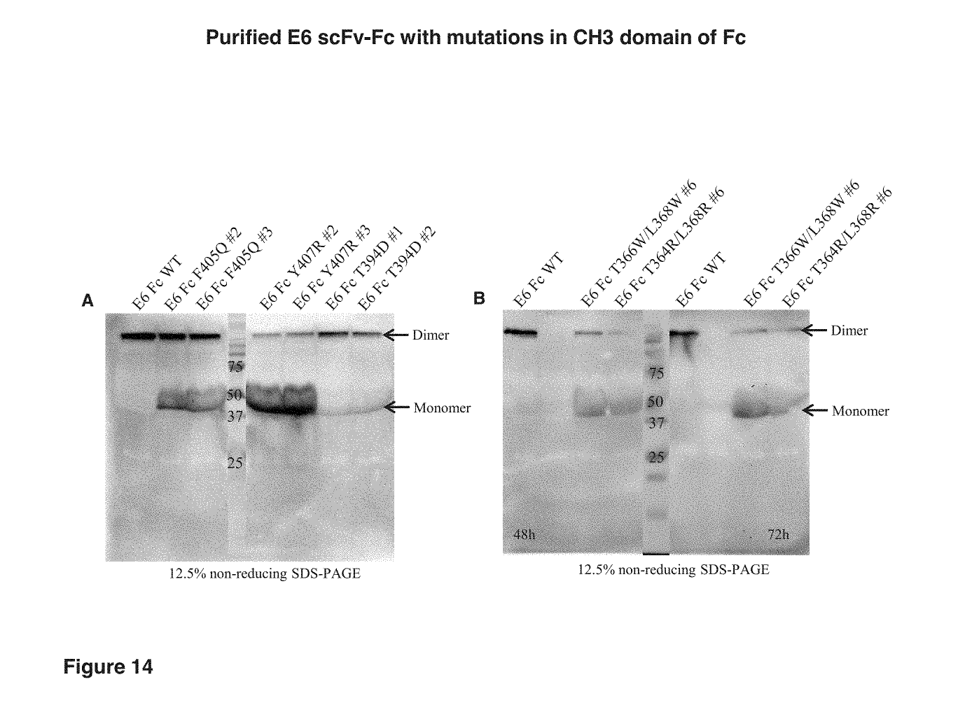

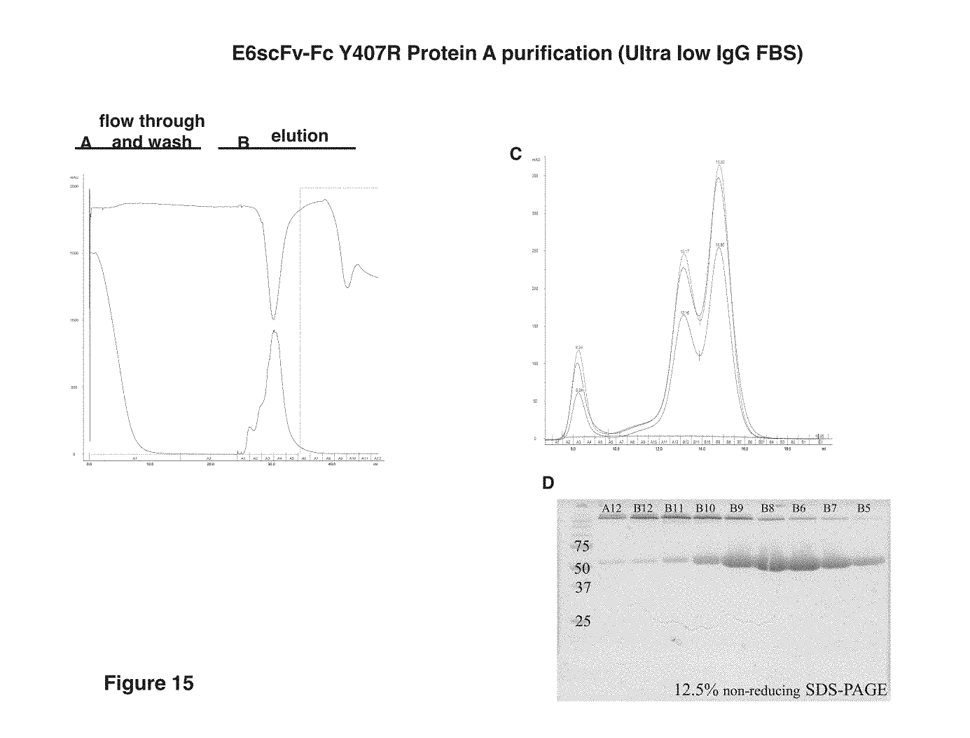

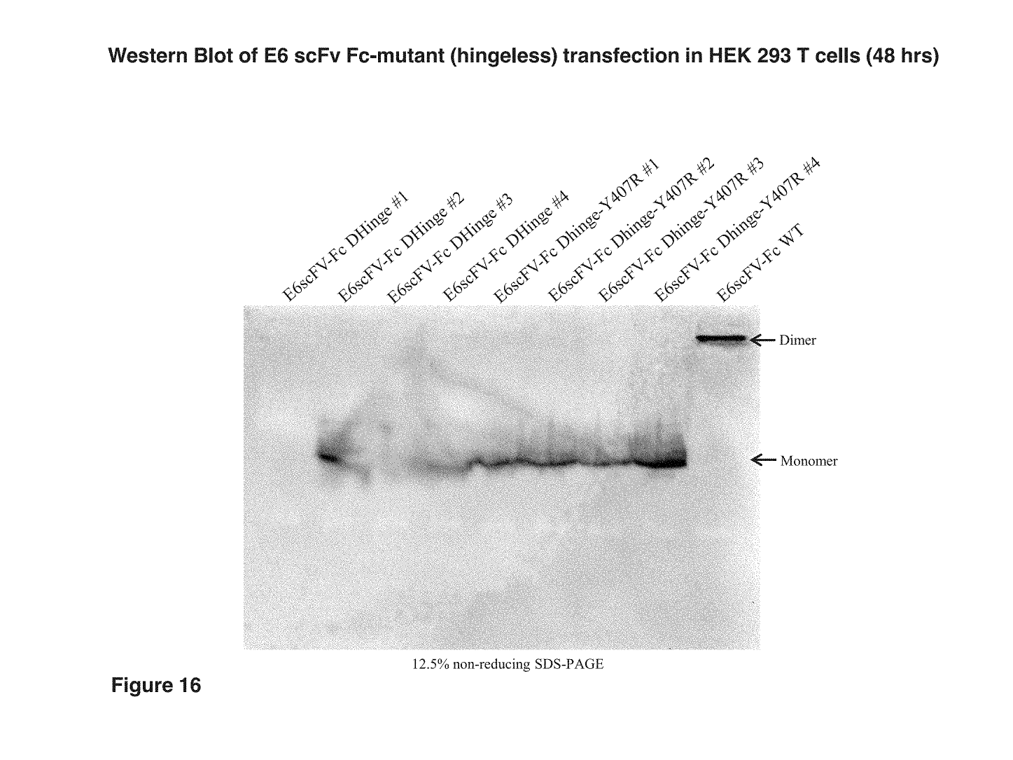

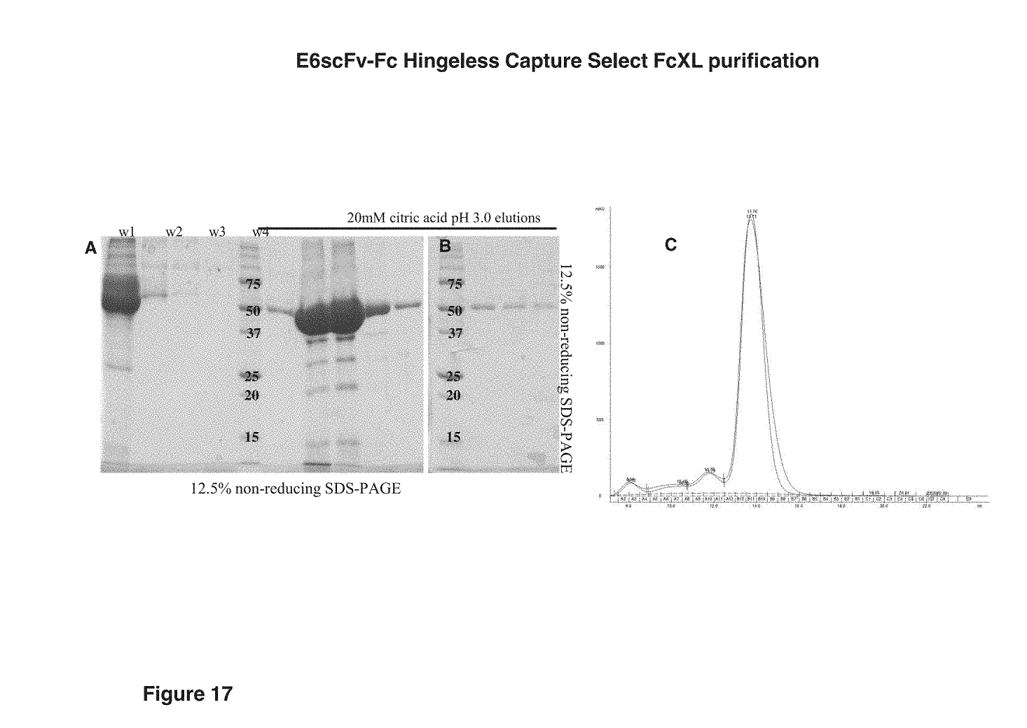

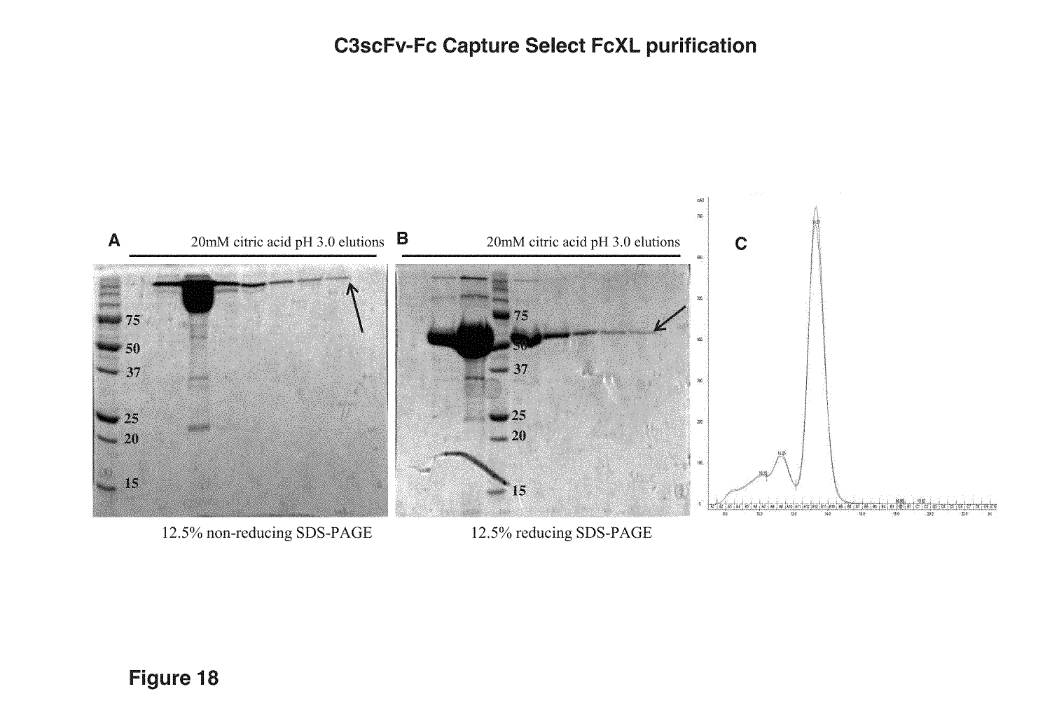

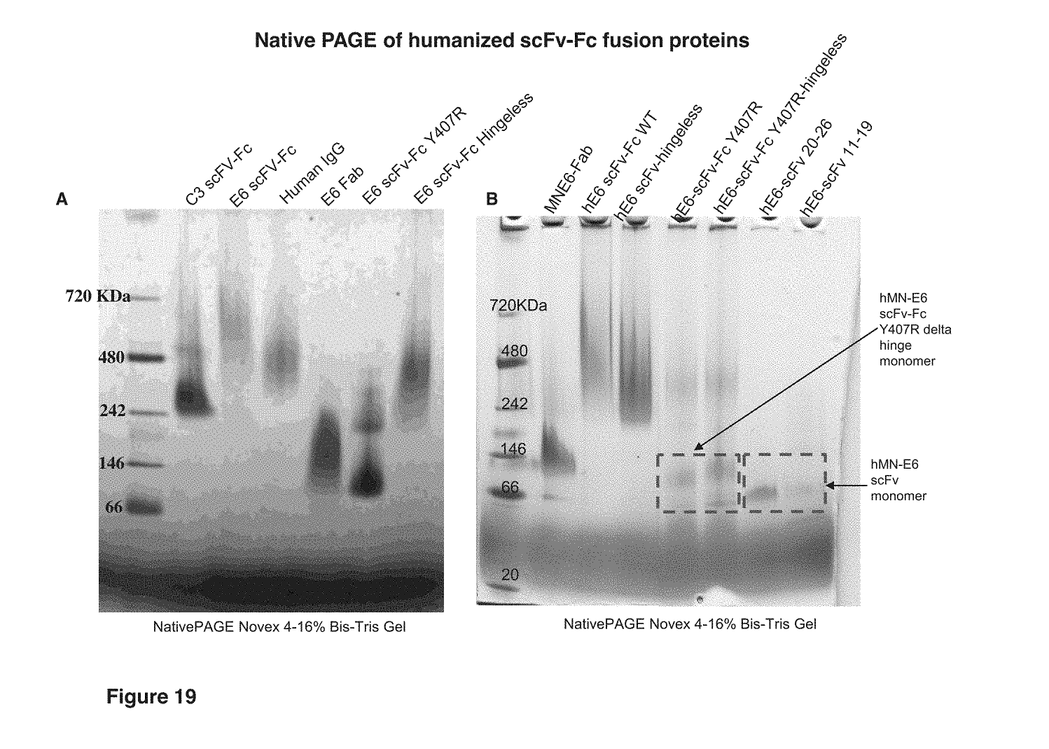

However, the Fc portion of an antibody homo-dimerizes, which in the case of anti-MUC1* antibody based therapeutics is not optimal since ligand-induced dimerization of the MUC1* receptor stimulates growth. As can be seen in FIG. 13 B, humanized MN-E6 scFv-Fc is a dimer, in part due to disulfide bonding. Therefore, mutations in the Fc region that resist dimer formation are preferred for anti-MUC1* anti-cancer therapeutics. Deletion of the hinge region (hingeless also called delta hinge or Dhinge in some figures and examples SEQ ID NO: 288 and 289) and other mutations in the Fc region that make the Fc-mutant resistant to dimerization were made. The following mutations were made in the CH3 domain to create a monomeric scFv-Fc fusion protein: Y407R (SEQ ID NO: 278 and 279), F405Q (SEQ ID NO: 280 and 281), T394D (SEQ ID NO: 282 and 283), T366W/L368W (SEQ ID NO: 284 and 285), T364R/L368R (SEQ ID NO: 286 and 287). FIG. 14 shows photographs of SDS-PAGE characterization of purified MN-E6 scFv-Fc fusion proteins on a non-reducing gels, wherein the Fc portion that was fused to the MN-E6 was either wild type (wt) or mutated as follows: A) F405Q, Y407R, T394D; B) T366W/L368W, T364R/L368R, T366W/L368W or T364R/L368R. Fc mutants F405Q, Y407R, T366W/L368W, T364R/L368R, T366W/L368W and T364R/L368R all favored monomer over dimer formation. FIG. 15 shows FPLC traces of the purification of MN-E6 scFv-Fc Y407Q fusion protein that was grown in low IgG FBS over a Protein A affinity column. A) is the trace of the flow through. B) is the trace of the elution. The protein was further purified by size exclusion over an S200 column (C). (D) is a photograph of an SDS-PAGE gel showing which fractions had a predominance of monomer. FIG. 16 shows a photograph of SDS-PAGE characterization of purified MN-E6 scFv-Fc-mutant fusion proteins on a non-reducing gel, wherein the Fc portion that was fused to the MN-E6 scFv was either wild type (wt) or mutated by elimination of the hinge region, ‘DHinge’, of the Fc or elimination of the hinge region of the Fc and also bearing the Y407R mutation. All the Fc mutants favored monomer over dimer formation. The reference construct amino acid sequence for the indicated mutation is SEQ ID NO:273. Other relevant sequences are SEQ ID NOS:289 and 279. FIG. 17A and FIG. 17B show photograph of non-reducing SDS-PAGE characterization of large scale expression and purification of MN-E6 scFv-Fc hingeless mutant, showing that it is a monomer. FPLC characterization and purification of MN-E6 scFv-Fc hingeless mutant is shown (FIG. 17C). FIGS. 18A-18C show photographs of the SDS-PAGE characterization of the purified MN-C3 scFv-Fc fusion protein on a non-reducing gel (FIG. 18A) or a reducing gel (FIG. 18B). The protein was purified by size exclusion. The FPLC trace is shown (FIG. 18C). FIGS. 19A-19B show photographs of Native gels of MN-C3 or MN-E6 Fabs, scFv, scFv-Fc, wherein the Fc portion is wild type or mutants that prefer or are exclusively monomers. Native gels show that the Y407R Fc mutation (FIG. 19A) and the double mutant Y407R and a deleted hinge (FIG. 19B) favor monomer over dimer the best. Note that proteins are loaded onto a gel at much higher concentrations than typical use concentrations. The dimer formation of other Fc mutants may only reflect the fact that loading concentration is very high.

Some mutations or deletions were so effective that, even when loaded onto a gel at high concentrations, they resist dimer formation (FIGS. 14A, 14B). The Y407R mutation results in a nearly pure population of dimeric scFv-Fc (FIG. 10). Similarly deletions of the hinge region of the Fc result in fusion proteins that are monomers rather than dimers. Combinations of mutations can result in even more effective resistance of dimer formation (FIGS. 16 and 17). These and other mutations and combinations thereof were introduced into CH2-CH3 (SEQ ID NO:274 and 275) and CH3 (SEQ ID NO:276 and 277) fusion proteins such as scFv or in the hingeless Fc-fusion proteins such as scFv and were shown to eliminate or minimize dimerization.

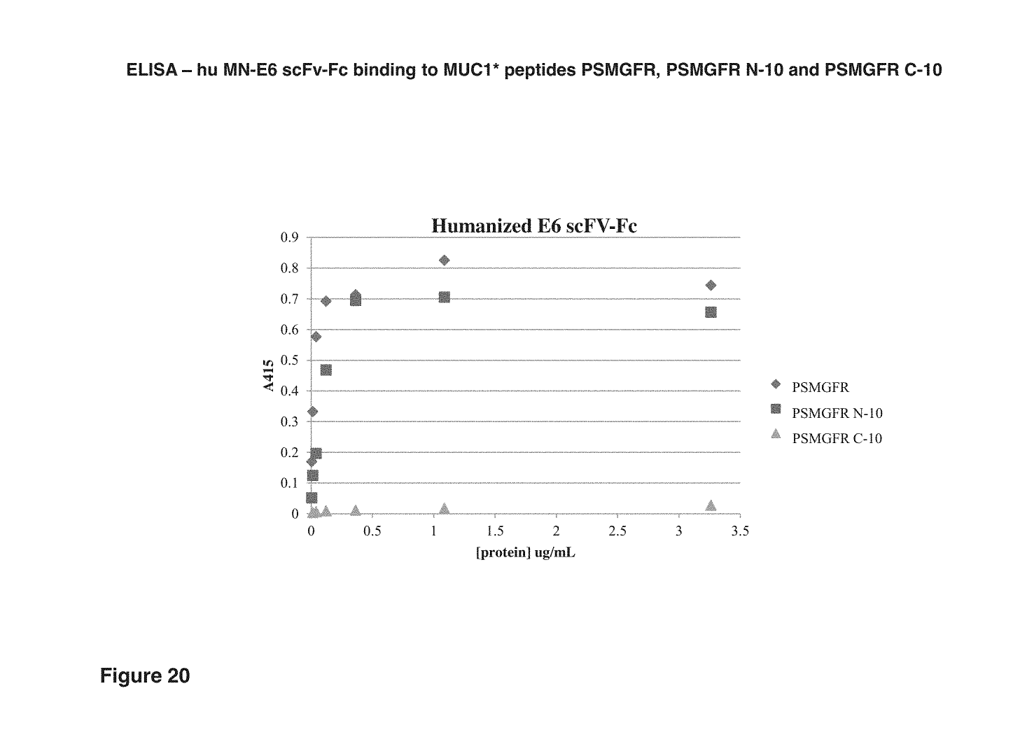

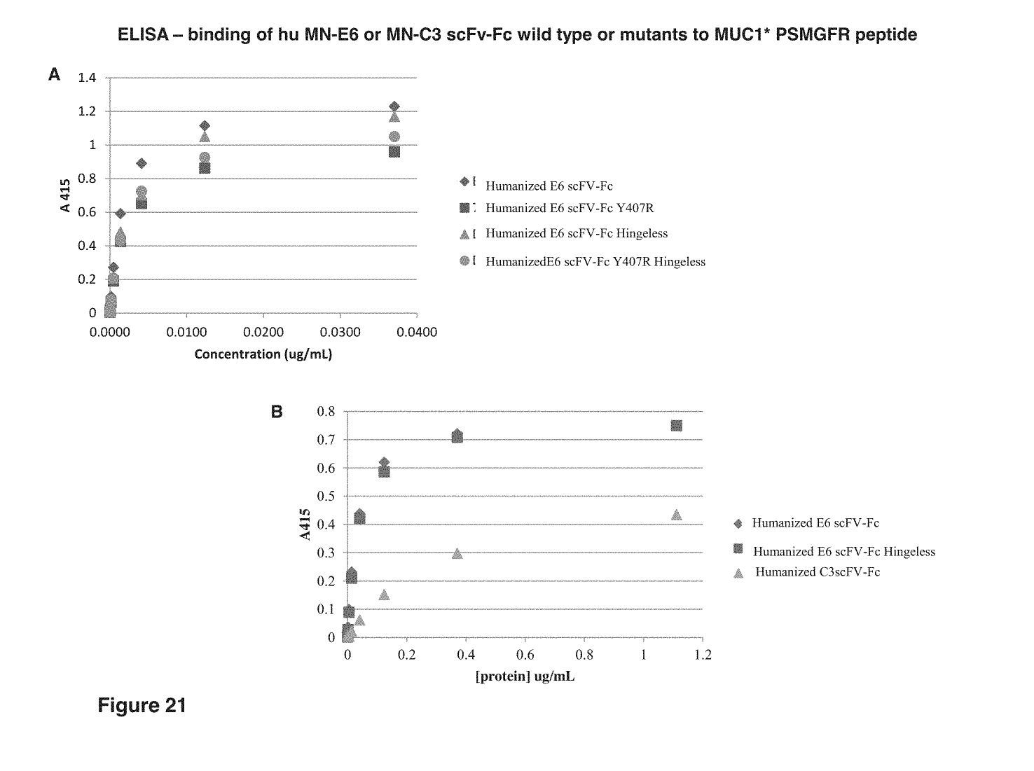

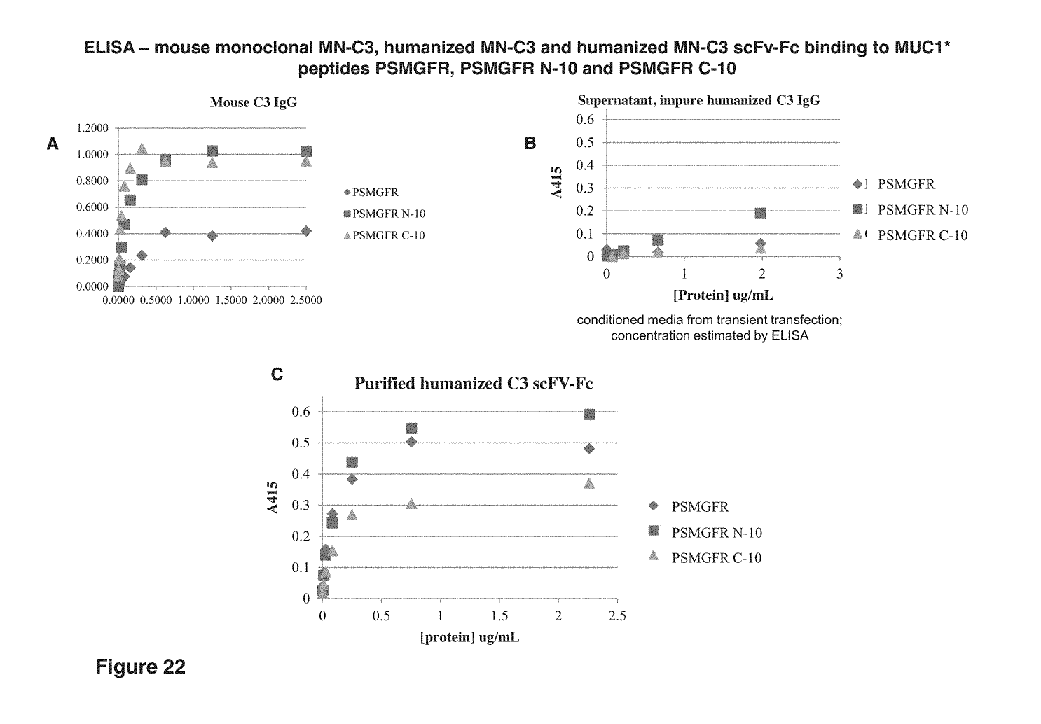

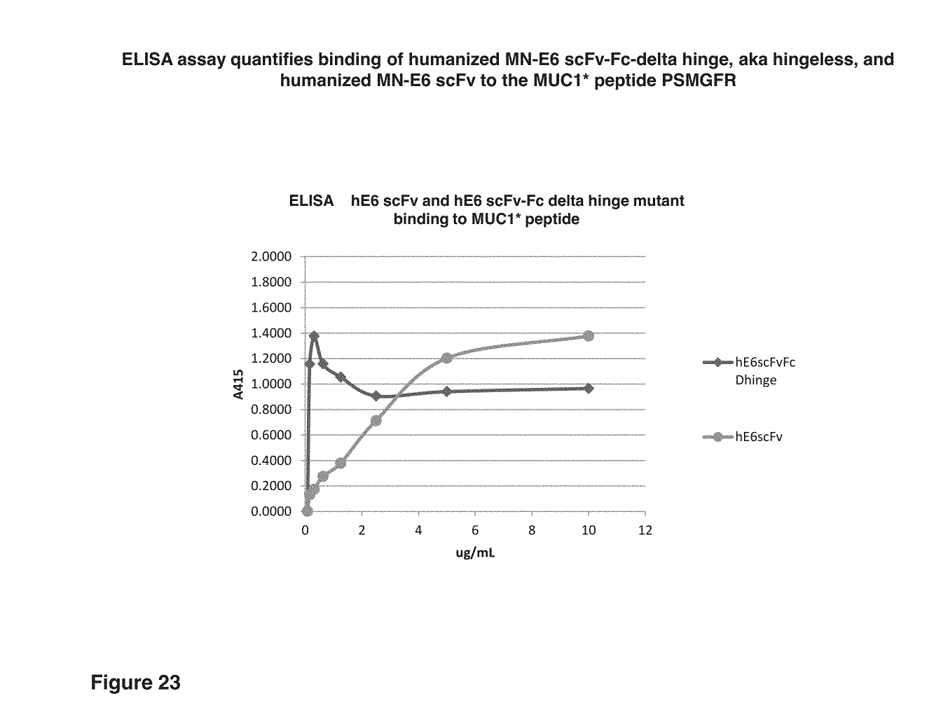

Like the parent mouse monoclonal antibodies, human or humanized antibodies as well as single chain constructs, scFv's, scFv-Fc fusions or scFv-Fc-mutants specifically bind to the synthetic MUC1* peptides (FIGS. 20-22). FIG. 23 shows a graph of an ELISA assay that quantifies the binding of humanized MN-E6 scFv-Fc-delta hinge, aka Dhinge or hingeless, and humanized MN-E6 scFv to the MUC1* peptide PSMGFR.

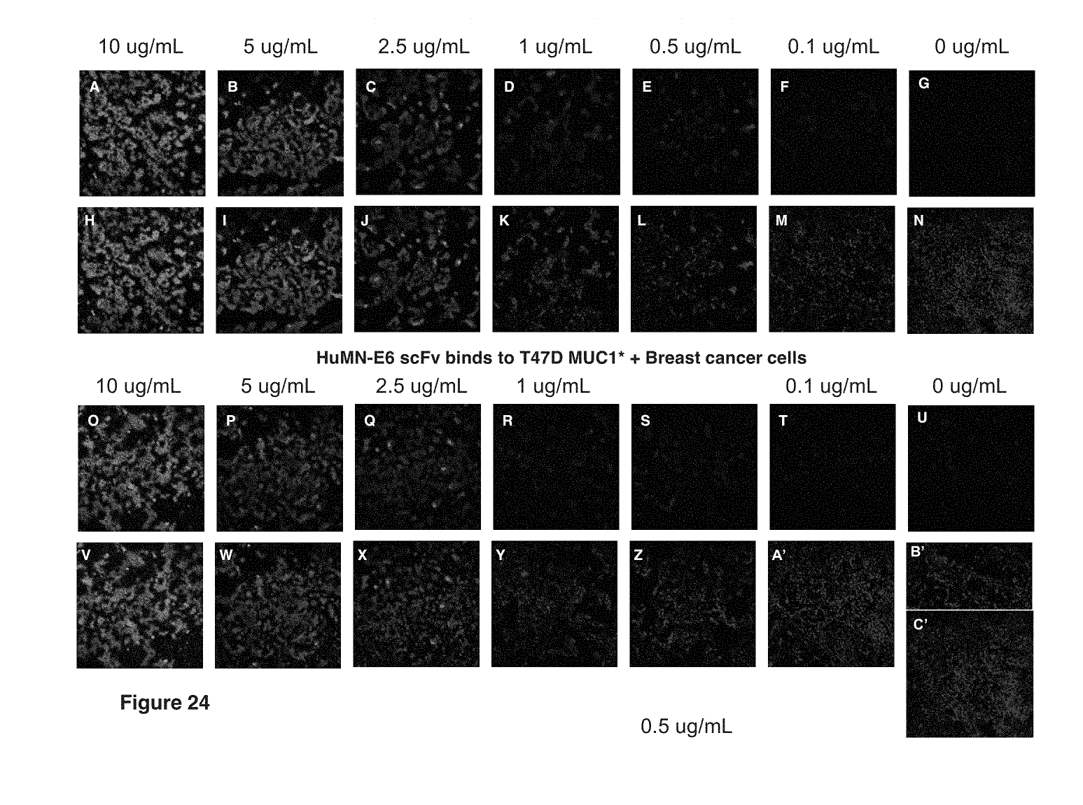

The human or humanized anti-MUC1* antibody fragments described here specifically bind to MUC1 and MUC1* positive cancer cells. FIG. 24 shows photographs of immunofluorescence experiments in which humanized MN-C2 scFv or MN-E6 scFv specifically binds to MUC1* positive breast cancer cells in an identical concentration dependent manner. A-G: hu MN-C2 scFv binding to T47D breast cancer cells at concentrations indicated. H—N shows the fluorescently labeled scFv and DAPI. O-U: hu MN-E6 scFv binding to T47D breast cancer cells at concentrations indicated. V-B′ shows the fluorescently labeled scFv and DAPI. C′ is the secondary antibody control.

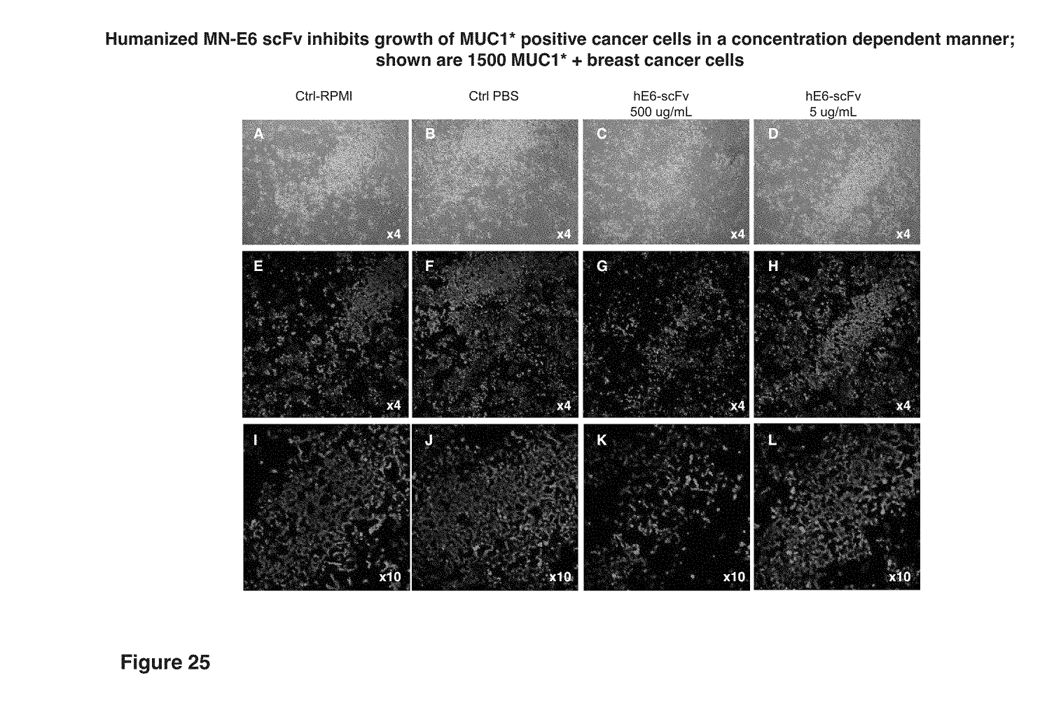

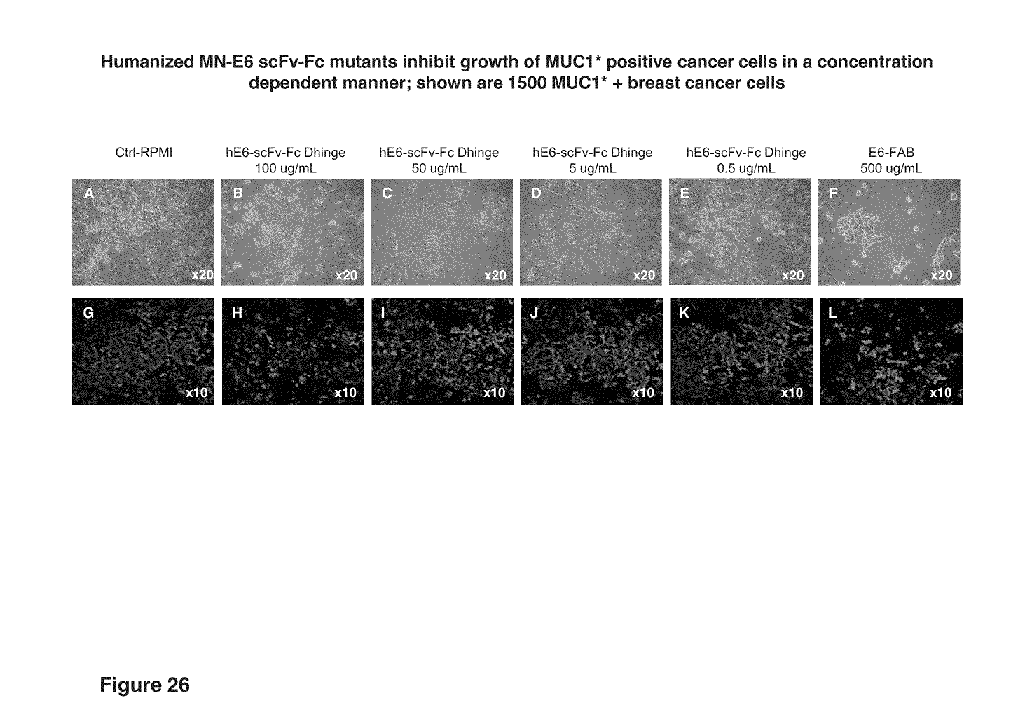

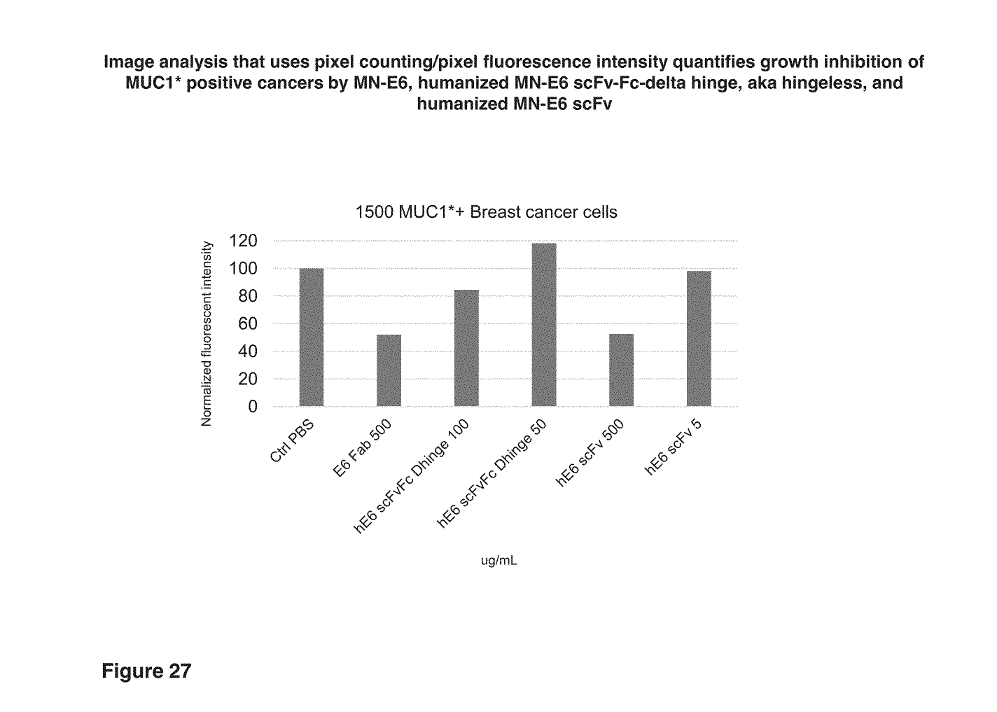

In addition to binding to MUC1* positive cancer cells, the anti-MUC1* antibody variable region fragments, scFv's, scFv-Fc's and scFv-Fc-mutants inhibited growth of MUC1-positive cancer cells. FIGS. 25A-25L show photographs of MUC1* positive breast cancer cells that have been cultured in normal medium or in the presence of humanized MN-E6 scFv. The photographs show killing and/or growth inhibition of MUC1* positive cells by MN-E6 scFv at 5 ug/mL and an even greater effect at 500 ug/mL. FIGS. 26A-26L show photographs of MUC1* positive breast cancer cells that have been cultured in normal medium or in the presence of humanized MN-E6 scFv-Fc Dhinge, which is a hingeless or delta hinge mutant. The photographs show killing and/or growth inhibition of MUC1* positive cells by hMN-E6 scFv-Fc Dhinge 5 ug/mL, an even greater effect at 50 ug/mL and yet an even greater effect at 100 ug/mL. FIG. 27 shows a graph of the image analysis of the fluorescent images of FIGS. 25 and 26. Image J was used to quantify the number of cells remaining after 96 hours treatment in humanized MN-E6scFv or MN-E6 scFv-Fc-delta hinge, aka Dhinge. The analysis software uses pixel counting and pixel fluorescence intensity to quantify the number of cells in each photograph. Analysis was performed over the entire image 512×512 pixels, 8-bit image. For comparison, the inhibition of mouse monoclonal MN-E6 Fab is also analyzed.

These data show that a human or humanized MN-E6 antibody or antibody fragment, Fab, MN-E6 scFv or hu MN-E6 scFv-Fcₘᵤₜ are effective anti-cancer agents that can be administered to a person diagnosed with a MUC1 or MUC1* positive cancer, suspected of having a MUC1 or MUC1* positive cancer or is at risk of developing a MUC1 or MUC1* positive cancer.

In these specific examples, the dimer resistant Fc that was fused onto an antibody fragment or scFv is hu MN-E7 scFv. However, any of these Fc region mutations or combinations thereof that eliminate or minimize dimerization can be fused onto variable region fragments or single chain constructs of MN-E6, MN-C2, MN-C3 or MN-C8 or other antibodies identified that selectively bind to MUC1* as it exists on cancer cells or tissues. In addition, the Fabs of these antibodies can be used as an anti-cancer therapeutic. In one aspect of the invention, a person diagnosed with, suspected of having or is at risk of developing a MUC1* or MUC1 positive cancer is treated with an effective amount of human or humanized MN-E6 scFv, MN-C2 scFv, MN-C3 scFv, or MN-C8 scFv. In another aspect of the invention, a person diagnosed with, suspected of having or is at risk of developing a MUC1* or MUC1 positive cancer is treated with an effective amount of human or humanized MN-E6 scFv-Fc_{Y407R}, MN-C2 scFv-Fc_{Y407R}, MN-C3 scFv-Fc_{Y407R}, or MN-C8 scFv-Fc_{Y407R}. In another aspect of the invention, a person diagnosed with, suspected of having or is at risk of developing a MUC1* or MUC1 positive cancer is treated with an effective amount of human or humanized MN-E6 scFv-Fc mutant_{Dhinge}, MN-C2 scFv-Fc mutant_{Dhinge}, MN-C3 scFv-Fc mutant_{Dhinge}, or MN-C8 scFv-Fc mutant_{Dhinge}. In yet another aspect of the invention, a person diagnosed with, suspected of having or is at risk of developing a MUC1* or MUC1 positive cancer is treated with an effective amount of human or humanized MN-E6 scFv-Fc mutant_{Y407R-Dhinge}, MN-C2 scFv-Fc mutant_{Y407R-Dhinge}, MN-C3 scFv-Fc mutant_{Y407R-Dhinge}, or MN-C8 scFv-Fc mutant_{Y407R-Dhinge}. One aspect of the invention is a method for treating a patient diagnosed with, suspected of having, or at risk of developing a MUC1 positive or MUC1* positive cancer, wherein the patient is administered an effective amount of a monomeric MN-E6 scFv, MN-C2 scFv, MN-C3 scFv, MN-C8 scFv, or MN-E6 scFv-Fc, MN-C2 scFv-Fc, MN-C3 scFv-Fc, MN-C8 scFv-Fc, wherein the Fc portion of the antibody-like protein has been mutated such that it resists dimer formation.

Humanizing

Humanized antibodies or antibody fragments or fully human antibodies that bind to the extracellular domain of −MUC1* are preferred for therapeutic use. The techniques described herein for humanizing antibodies are but a few of a variety of methods known to those skilled in the art. The invention is not meant to be limited by the technique used to humanize the antibody.

Humanization is the process of replacing the non-human regions of a therapeutic antibody (usually mouse monoclonal antibody) by human one without changing its binding specificity and affinity. The main goal of humanization is to reduce immunogenicity of the therapeutic monoclonal antibody when administered to human. Three distinct types of humanization are possible. First, a chimeric antibody is made by replacing the non-human constant region of the antibody by the human constant region. Such antibody will contain the mouse Fab region and will contain about 80-90% of human sequence. Second, a humanized antibody is made by grafting of the mouse CDR regions (responsible of the binding specificity) onto the variable region of a human antibody, replacing the human CDR (CDR-grafting method). Such antibody will contain about 90-95% of human sequence. Third and last, a full human antibody (100% human sequence) can be created by phage display, where a library of human antibodies is screened to select antigen specific human antibody or by immunizing transgenic mice expressing human antibody.

A general technique for humanizing an antibody is practiced approximately as follows. Monoclonal antibodies are generated in a host animal, typically in mice. Monoclonal antibodies are then screened for affinity and specificity of binding to the target. Once a monoclonal antibody that has the desired effect and desired characteristics is identified, it is sequenced. The sequence of the animal-generated antibody is then aligned with the sequences of many human antibodies in order to find human antibodies with sequences that are the most homologous to the animal antibody. Biochemistry techniques are employed to paste together the human antibody sequences and the animal antibody sequences. Typically, the non-human CDRs are grafted into the human antibodies that have the highest homology to the non-human antibody. This process can generate many candidate humanized antibodies that need to be tested to identify which antibody or antibodies has the desired affinity and specificity.

Once a human antibody or a humanized antibody has been generated it can be further modified for use as an Fab fragment, as a full antibody, or as an antibody-like entity such as a single chain molecule containing the variable regions, such as scFv or an scFv-Fc. In some cases it is desirable to have Fc region of the antibody or antibody-like molecule mutated such that it does not dimerize.

In addition to methods that introduce human sequences into antibodies generated in non-human species, fully human antibodies can be obtained by screening human antibody libraries with a peptide fragment of an antigen. A fully human antibody that functions like MN-E6 or MN-C2 is generated by screening a human antibody library with a peptide having the sequence of the PSMGFR N-10 peptide. A fully human antibody that functions like MN-C3 or MN-C8 is generated by screening a human antibody library with a peptide having the sequence of the PSMGFR C-10 peptide.

Humanized anti-MUC1* antibodies were generated based on the sequences of the mouse monoclonal antibodies MN-E6, MN-C2, MN-C3 and MN-C8. In one aspect of the invention, a patient diagnosed with a MUC1* positive cancer is treated with an effective amount of humanized MN-E6, MN-C2, MN-C3 or MN-C8. In a preferred embodiment, a patient diagnosed with a MUC1* positive cancer is treated with an effective amount of humanized MN-E6 or MN-C2. In another aspect of the invention, a patient diagnosed with a MUC1* positive cancer is treated with an effective amount of humanized monovalent MN-E6, MN-C2, MN-C3 or MN-C8, wherein monovalent means the corresponding Fab fragment, the corresponding scFv or the corresponding scFv-Fc fusion. In a preferred embodiment, a patient diagnosed with a MUC1* positive cancer is treated with an effective amount of a humanized scFv or monomeric humanized scFv-Fc of MN-E6 or MN-C2. Since the MUC1* growth factor receptor is activated by ligand induced dimerization of its extracellular domain, and because the Fc portion of an antibody homo-dimerizes, it is preferable that a construct that includes an Fc portion uses a mutated Fc region that prevents or minimizes dimerization.

Antibodies that bind to PSMGFR (SEQ ID NO:2) peptide of the extracellular domain of the MUC1* receptor are potent anti-cancer therapeutics that are effective for the treatment or prevention of MUC1* positive cancers. They have been shown to inhibit the binding of activating ligands dimeric NME1 (SEQ ID NOS: 3 and 4) and NME7 (SEQ ID NOS: 5 and 6) to the extracellular domain of MUC1*. Anti-MUC1* antibodies that bind to the PSMGFR sequence inhibit the growth of MUC1*-positive cancer cells, specifically if they inhibit ligand-induced receptor dimerization. Fabs of anti-MUC1* antibodies have been demonstrated to block tumor growth in animals. Thus, antibodies or antibody fragments that bind to the extracellular domain of MUC1* would be beneficial for the treatment of cancers wherein the cancerous tissues express MUC1*.

Antibodies that bind to PSMGFR region of MUC1* or bind to a synthetic PSMGFR peptide are preferred. We have identified several monoclonal antibodies that bind to the extracellular domain of MUC1*. Among this group are mouse monoclonal antibodies MN-E6, MN-C2, MN-C3 and MN-C8, the variable regions of which were sequenced and are given as for MN-E6 SEQ ID NOS: 12-13 and 65-66, for MN-C2 SEQ ID NOS: 118-119 and 168-169, for MN-C3 SEQ ID NOS: 413-414 and 458-459 and for MN-C8 SEQ ID NOS: 505-506 and 543-554. The CDRs of these antibodies make up the recognition units of the antibodies and are the most important parts of the mouse antibody that should be retained when grafting into a human antibody. The sequences of the CDRs for each mouse monoclonal are as follows, heavy chain sequence followed by light chain: MN-E6 CDR1 (SEQ ID NO:16-17 and 69-70) CDR2 (SEQ ID NO:20-21 and 73-74) CDR3 (SEQ ID NO: 24-25 and 77-78), MN-C2 CDR1 (SEQ ID NO:122-123 and 172-173) CDR2 (SEQ ID NO:126-127 and 176-177) CDR3 (SEQ ID NO:130-131 and 180-181), MN-C3 CDR1 (SEQ ID NO:417-418 and 462-463) CDR2 (SEQ ID NO:421-422 and 466-467) CDR3 (SEQ ID NO:425-426 and 470-471), MN-C8 CDR1 (SEQ ID NO:507-508 and 545-546) CDR2 (SEQ ID NO:509-510 and 547-548) CDR3 (SEQ ID NO:511-512 and 549-550). In some cases, portions of the framework regions that by modeling are thought to be important for the 3-dimensional structure of the CDRs, are also imported from the mouse sequence.

Monoclonal antibodies MN-E6 and MN-C2 have greater affinity for MUC1* as it appears on cancer cells. Monoclonal antibodies MN-C3 and MN-C8 have greater affinity for MUC1* as it appears on stem cells. By sequence alignment the following human antibodies were chosen as being sufficiently homologous to the mouse antibody that substitution of the mouse CDRs would result in an antibody that retained ability to recognize the target. Mouse MN-E6 heavy chain variable region was homologous to human IGHV3-21*03 heavy chain variable region (SEQ ID NO: 26-27) and the light chain variable region was homologous to human IGKV3-11*02 light chain variable region (SEQ ID NO: 79-80). Mouse MN-C2 heavy chain variable region was homologous to human IGHV3-21*04 heavy chain variable region (SEQ ID NO: 132-133) and the light chain variable region was homologous to human IGKV7-3*01 light chain variable region (SEQ ID NO: 182-183). Mouse MN-C3 heavy chain variable region was homologous to human IGHV1-18*04 heavy chain variable region (SEQ ID NO: 427-428) and the light chain variable region was homologous to human IGKV2-29*03 light chain variable region (SEQ ID NO:472-473). Mouse MN-C8 heavy chain variable region was homologous to human IGHV3-21*04 heavy chain variable region (SEQ ID NO: 513-514) and the light chain variable region was homologous to human Z00023 light chain variable region (SEQ ID NO:551-552).

All four antibodies have been humanized, which process has resulted in several humanized forms of each antibody. CDRs derived from the variable regions of the mouse antibodies were biochemically grafted into a homologous human antibody variable region sequence. Humanized variable regions of MN-E6 (SEQ ID NOS: 38-39 and 93-94), MN-C2 (SEQ ID NOS: 144-145 and 194-195), MN-C3 (SEQ ID NOS: 439-440 and 486-487) and MN-C8 (SEQ ID NOS: 525-526 and 543-544) were generated by grafting the mouse CDRs into the variable region of a homologous human antibody. The humanized heavy chain variable constructs were then fused into constant regions of either human IgG1 heavy chain constant region (SEQ ID NOS:58-59) or human IgG2 heavy chain constant region (SEQ ID NO:54-55), which are then paired with either humanized light chain variable constructs fused to a human kappa chain (SEQ ID NO: 109-110) or human lambda chain (SEQ ID NO: 113-114) constant region. Other IgG isotypes could be used as constant region including IgG3 or IgG4.

Examples of humanized MN-E6 variable region into an IgG2 heavy chain (SEQ ID NOS:52-53) and into an IgG1 heavy chain (SEQ ID NOS:56-57), humanized MN-C2 variable into an IgG1 heavy chain (SEQ ID NOS: 158-159) or into an IgG2 heavy chain (SEQ ID NOS: 163-164) paired with either Lambda light chain (SEQ ID NO: 111-112 and 216-219) or Kappa chain (SEQ ID NO:107-108 and 210-213) and, humanized MN-C3 (SEQ ID NOS: 455-456, 453-454 and 500-501, 502-503) and MN-C8 (SEQ ID NOS: 541-542, 539-540 and 579-580, 581-582) antibodies were generated. Which IgG constant region is fused to the humanized variable region depends on the desired effect since each isotype has its own characteristic activity. The isotype of the human constant region is selected on the basis of things such as whether antibody dependent cell cytotoxicity (ADCC) or complement dependent cytotoxicity (CDC) is desired but can also depend on the yield of antibody that is generated in cell-based protein expression systems. In a preferred embodiment, humanized anti-MUC1* antibodies or antibody fragments are administered to a person diagnosed with or at risk of developing a MUC1-positive cancer.

One method for testing and selecting the humanized anti-MUC1* antibodies that would be most useful for the treatment of persons with cancer or at risk of developing cancers is to test them for their ability to inhibit the binding of activating ligands to the MUC1* extracellular domain. Dimeric NME1 can bind to and dimerize the MUC1* extracellular domain and in so doing stimulates cancer cell growth. Antibodies and antibody fragments that compete with NME1 for binding to the MUC1* extracellular domain are therefore anti-cancer agents. NME7 is another activating ligand of MUC1*. In some cases, it is preferable to identify antibodies that block the binding of NME7, or an NME7 truncation or cleavage product, to the MUC1* extracellular domain. Antibodies and antibody fragments that compete with NME7 and NME7 variants for binding to the MUC1* extracellular domain are effective as anti-cancer therapeutics. These antibodies include but are not limited to MN-E6, MN-C2, MN-C3, MN-C8 as well as single chain versions, such as scFv, of these antibodies and humanized version thereof. Other NME proteins also bind to MUC1 or MUC1* including NME6 and NME8. Antibodies that compete with these proteins for binding to MUC1* may also be useful as therapeutics. In a preferred embodiment, humanized anti-MUC1* antibodies or antibody fragments are administered to a person diagnosed with or at risk of developing a MUC1-positive cancer. In a more preferred embodiment, single chain antibody fragments, or monomeric scFv-Fc fusions, derived from humanized sequences of MN-E6 and MN-C2 are administered to a person diagnosed with or at risk of developing a MUC1-positive cancer.

Single chain variable fragments, scFv, or other forms that result in a monovalent antibody or antibody-like protein are also useful. In some cases it is desired to prevent dimerization of the MUC1* extracellular domain. Single chain variable fragments, Fabs and other monovalent antibody-like proteins have been shown to be effective in binding to the extracellular domain of MUC1* and blocking MUC1* dimerization. These single chain variable fragments, Fabs and other monovalent antibody-like molecules effectively blocked cancer growth in vitro and in animals xenografted with human MUC1-positive cancer cells. Thus, humanized single chain variable fragments or monovalent anti-MUC1* antibodies or antibody-like molecules would be very effective as an anti-cancer therapeutic. Such humanized single chain antibodies, Fabs and other monovalent antibody-like molecules that bind to the MUC1* extracellular domain or to a PSMGFR peptide are therefore useful as anti-cancer therapeutics. Anti-MUC1* single chain variable fragments are generated by grafting non-human CDRs of antibodies, which bind to extracellular domain of MUC1* or bind to PSMGFR peptide, into a framework of a homologous variable region human antibody. The resultant humanized heavy and light chain variable regions are then connected to each other via a suitable linker, wherein the linker should be flexible and of length that it allows heavy chain binding to light chain but discourages heavy chain of one molecule binding to the light chain of another. For example a linker of about 10-15 residues. Preferably, the linker includes [(Glycine)₄ (Serine)₁]₃ (SEQ ID NOS: 401-402), but is not limited to this sequence as other sequences are possible.

In one aspect, the humanized variable regions of MN-E6 (SEQ ID NOS: 38-39 and 93-94), MN-C2 (SEQ ID NOS: 144-145 and 194-195), MN-C3 (SEQ ID NOS: 439-440 and 486-487) and MN-C8 (SEQ ID NOS: 525-526 and 565-566) are biochemically grafted into a construct that connects heavy and light chains via a linker. Examples of humanized single chain anti-MUC1* antibodies comprising humanized sequences from the variable regions of MN-E6, MN-C2, MN-C3 and MN-C8 were generated. Several humanized MN-E6 single chain proteins were generated (SEQ ID NOS: 232-237). Several humanized MN-C2 single chain proteins were generated (SEQ ID NOS: 238-243). Several humanized MN-C3 single chain proteins were generated (SEQ ID NOS: 244-249). Several humanized MN-C8 single chain proteins were generated (SEQ ID NOS: 250-255). In a preferred embodiment, humanized anti-MUC1* antibody fragments, including variable fragments, scFv antibody fragments MN-E6 scFv, MN-C2 scFv, MN-C3 scFv, or MN-C8 scFv are administered to a person diagnosed with or at risk of developing a MUC1-positive cancer. In a more preferred embodiment, single chain antibody fragments, such as variable fragments derived from humanized sequences of MN-E6 and MN-C2, are administered to a person diagnosed with or at risk of developing a MUC1-positive cancer.

In another aspect, the humanized variable regions of MN-E6 (SEQ ID NOS: 38-39 and 93-94), MN-C2 (SEQ ID NOS: 144-145 and 194-195), MN-C3 (SEQ ID NOS: 439-440 and 486-487) and MN-C8 (SEQ ID NOS: 525-526 and 565-566) are biochemically grafted into a single chain variable fragment, scFv, that also contains an Fc portion of an antibody. Examples of humanized single chain variable fragment of MN-E6, MN-C2, MN-C3 and MN-C8 fused to a Fc region of an antibody were generated (SEQ ID NOS: 256-257, 260-261, 264-265 and 268-269). Inclusion of an Fc region serves several purposes. It increases the molecular weight of the antibody fragment, which slows degradation and increases half-life. An Fc region also recruits immune system complement to the tumor site. Additionally, the addition of an antibody Fc region makes the scFv a convenient diagnostic tool, as the secondary antibodies detect and label the Fc portion. However, the Fc portion homo-dimerizes. Thus an scFv-Fc would be bivalent and could dimerize and activate the MUC1* growth factor receptor. In order to get the benefits of having an Fc attached to an anti-MUC1* scFv, without the drawback of inducing MUC1* dimerization, the Fc region was mutated to minimize or eliminate Fc homo-dimerization. The following mutations were made in the CH3 domain to create a monomeric scFv-Fc fusion protein: Y407R (SEQ ID NOS: 278 and 279), F405Q (SEQ ID NOS: 280 and 281), T394D (SEQ ID NOS: 282 and 283), T366W/L368W (SEQ ID NOD: 284 and 285), T364R/L368R (SEQ ID NOS: 286 and 285). Any combinations of those mutations can be tested and could be introduced into Fc (SEQ ID NOS: 272-273), CH2-CH3 (SEQ ID NOS: 274-275) or CH3 (SEQ ID NOS: 276-277) fusion proteins or in the hingeless Fc-fusion proteins (SEQ ID NOS: 288-289).

One aspect of the invention is a method for treating a patient diagnosed with, suspected of having, or at risk of developing a MUC1 positive or MUC1* positive cancer, wherein the patient is administered an effective amount of a monomeric MN-E6 scFv, MN-C2 scFv, MN-C3 scFv, MN-C8 scFv, or MN-E6 scFv-Fc, MN-C2 scFv-Fc, MN-C3 scFv-Fc, MN-C8 scFv-Fc, wherein the antibody variable fragment portions are human or have been humanized and wherein the Fc portion of the antibody-like protein has been mutated such that it resists dimer formation.

CAR T and Cancer Immunotherapy Techniques

In another aspect of the invention, some or all of the single chain portions of anti-MUC1* antibody fragments are biochemically fused onto immune system molecules, using several different chimeric antigen receptor, ‘CAR’ strategies. The idea is to fuse the recognition portion of an antibody, typically as a single chain variable fragment, to an immune system molecule that has a transmembrane domain and a cytoplasmic tail that is able to transmit signals that activate the immune system. The recognition unit can be an antibody fragment, a single chain variable fragment, scFv, or a peptide. In one aspect, the recognition portion of the extracellular domain of the CAR is comprised of sequences from the humanized variable region of MN-E6 (SEQ ID NOS: 38-39 and 93-94), MN-C2 (SEQ ID NOS: 144-145 and 194-195), MN-C3 (SEQ ID NOS: 439-440 and 486-487) and MN-C8 (SEQ ID NOS: 525-526 and 565-566). In another aspect, it is comprised of sequences from a single chain variable fragment. Examples of single chain constructs are given. Several humanized MN-E6 single chain proteins, scFv, were generated (SEQ ID NOS: 232-237). Several humanized MN-C2 single chain proteins, scFv, were generated (SEQ ID NOS: 238-243). Several humanized MN-C3 single chain proteins, scFv, were generated (SEQ ID NOS: 244-249). Several humanized MN-C8 single chain proteins, scFv, were generated (SEQ ID NOS: 250-255). The transmembrane region of the CAR can be derived from CD8, CD4, antibody domains or other transmembrane region, including the transmembrane region of the proximal cytoplasmic co-stimulatory domain, such as CD28, 4-1BB or other. The cytoplasmic tail of the CAR can be comprised of one or more motifs that signal immune system activation. This group of cytoplasmic signaling motifs, sometimes referred to as, co-stimulatory cytoplasmic domains, includes but is not limited to CD3-zeta, CD27, CD28, 4-1BB, OX40, CD30, CD40, ICAm-1, LFA-1, ICOS, CD2, CD5, CD7 and Fc receptor gamma domain. A minimal CAR may have the CD3-zeta or an Fc receptor gamma domain then one or two of the above domains in tandem on the cytoplasmic tail. In one aspect, the cytoplasmic tail comprises CD3-zeta, CD28, 4-1BB and/or OX40.

Table 1 lists many of the anti-MUC1* CARs that we generated and tested. Several examples of MN-E6 CARs were generated: CAR MN-E6 CD3z (SEQ ID NOS: 294-295); CAR MN-E6 CD28/CD3z (SEQ ID NOS: 297-298); CAR MN-E6 4-1BB/CD3z (SEQ ID NOS: 300-301); CAR MN-E6 OX40/CD3z (SEQ ID NOS: 616-617); CAR MN-E6 CD28/OX40/CD3z (SEQ ID NOS: 618-619); CAR MN-E6 CD28/4-1BB/CD3z (SEQ ID NOS: 303-304). Several examples of humanized MN-C2 CARs were generated: CAR MN-C2 CD3z (SEQ ID NOS: 606-607); CAR MN-C2 CD28/CD3z (SEQ ID NOS: 608-609); CAR MN-C2 4-1BB/CD3z (SEQ ID NOS: 610-611); CAR MN-C2 OX40/CD3z (SEQ ID NOS: 612-613); CAR MN-C2 CD28/4-1BB/CD3z (SEQ ID NOS: 306-307); CAR MN-C2 CD28/OX40/CD3z (SEQ ID NOS: 614-615). Humanized MN-C3 CAR was generated: CAR MN-C3 4-1BB/CD3z (SEQ ID NOS: 600-601).

Several examples of humanized MN-E6 CARs with different hinge regions (SEQ ID NOS:345-360) were generated: CAR MN-E6-Fc/8/41BB/CD3z (SEQ ID NOS:310-311); CAR MN-E6 FcH/8/41BB/CD3z (SEQ ID NOS:315-316); CAR MN-E6 Fc/4/41BB/CD3z (SEQ ID NOS:318-319); CAR MN-E6 FcH/4/41BB/CD3z (SEQ ID NOS:321-322); CAR MN-E6 IgD/8/41BB/CD3z (SEQ ID NOS:323-324); CAR MN-E6 IgD/4/41BB/CD3z (SEQ ID NOS:327-328); CAR MN-E6 X4/8/41BB/CD3z (SEQ ID NOS:330-331); CAR MN-E6 X4/4/41BB/CD3z (SEQ ID NOS:333-334); CAR MN-E6 8+4/4/41BB/CD3z (SEQ ID NOS:336-337). In addition, several humanized MN-C3 single chain variable fragment and humanized MN-C8 single chain variable fragments were also generated.

Several CARs were also generated and tested wherein the targeting head of the CAR was derived from the anti-MUC1* antibody MNC2. CAR MN-C2-Fc/41BB/CD3z (SEQ ID NOS:732-733); CAR-MN-C2 IgD/Fc/4-1BB/CD3z (SEQ ID NOS:734-735); CAR MN-C2 FcH/41BB/CD3z (SEQ ID NOS:736-737); CAR-MN-C2 IgD/FcH/4-1BB/CD3z (SEQ ID NOS:738-739); CAR MN-C2 IgD/41BB/CD3z (SEQ ID NOS:740-741); CAR MN-C2 X4/41BB/CD3z (SEQ ID NOS:742-743).

The extracellular domain recognition unit of a MUC1* targeting CAR can comprise variable regions of any non-human, humanized or human antibody that is able to bind to at least 12 contiguous amino acids of the PSMGFR peptide (SEQ ID NO:2). In one aspect, the MUC1* targeting portion of the CAR comprises variable regions from non-human, humanized or human MN-E6, MN-C2, MN-C3 or MN-C8. In one aspect, the extracellular domain recognition unit of a CAR is comprised essentially of a humanized MN-E6, MN-C2, MN-C3 or MN-C8 single chain variable fragment scFv. The transmembrane region of the CAR can be derived from CD8 (SEQ ID NOS:363-364), or can be the transmembrane domain of CD3-zeta, CD28, 41bb, OX40 or other transmembrane region (SEQ ID NOS:361-372) and the cytoplasmic domain of a CAR with antibody fragment targeting MUC1* extracellular domain can be comprised of one or more selected from the group comprising an immune system co-stimulatory cytoplasmic domain. The group of immune system co-stimulatory domains includes but is not limited to CD3-zeta, CD27, CD28, 4-1BB, OX40, CD30, CD40, ICAm-1, LFA-1, ICOS, CD2, CD5, CD7 and Fc receptor gamma domain (SEQ ID NOS:373-382). Alternatively, the recognition unit portion of a CAR can comprise a peptide wherein the peptide binds to the target. NME7 binds to and activates MUC1*. In one aspect of the invention, the recognition unit of a CAR is a peptide derived from NME7 (SEQ ID NOS: 5-6) or a peptide derived from NME7, including but not limited to NME7 peptide A1 (SEQ ID NO: 7), NME7 peptide A2 (SEQ ID NO: 8), NME7 peptide B1 (SEQ ID NO: 9), NME7 peptide B2 (SEQ ID NO: 10) and NME7 peptide B3 (SEQ ID NO: 11).

Some strategies for generating CARs include a portion of the molecule that dimerizes with itself. In some cases, dimerization of the target is not desirable. Therefore, CARs can be constructed such that they heterodimerize. In one case the recognition unit of the first CAR binds to a first target while the recognition unit of the second CAR binds to a second target. Both recognition units can be antibody fragments, both can be peptides or one can be an antibody fragment and the other a peptide. A first target of the CAR can be the extracellular domain of MUC1*. The recognition unit of the CAR would be comprised of an antibody fragment that binds to MUC1* extracellular domain or to a PSMGFR peptide. Alternatively, the recognition unit of the CAR would be comprised of a peptide that binds to MUC1* extracellular domain, such peptides include peptides derived from an NME protein such as NME1 or NME7, more particularly NME7 derived peptides listed as SEQ ID NOS: 7-11. A second target of a heterodimeric CAR may be a peptide or antibody fragment that binds to NME7. Alternatively, a second target of a heterodimeric CAR may be a peptide or antibody fragment that binds to PD1 or its cognate ligand PDL-1 or other target ligand of the target cancer cell. A second target may be a peptide or antibody fragment that binds to NME1 or NME7-AB. Because it is desirable to prevent dimerization of MUC1 induced by a CAR, heterodimeric CARs can be constructed so that only the extracellular domain of one molecule has an extracellular recognition unit that binds to a target (SEQ ID NOS:584-587). The other molecule can have a truncated extracellular domain that is devoid of a target recognition unit or antibody fragment (SEQ ID NOS:588-599).

The CARs described can be transfected or transduced into a cell of the immune system. In a preferred embodiment, a MUC1* targeting CAR is transfected or transduced into a T cell. In one aspect, the T cell is a CD3+/CD28+ T cell. In another case it is a dendritic cell. In another case it is a B cell. In another case it is a mast cell. The recipient cell can be from a patient or from a donor. If from a donor, it can be engineered to remove molecules that would trigger rejection. Cells transfected or transduced with a CAR of the invention can be expanded ex vivo or in vitro then administered to a patient. Administrative routes are chosen from a group containing but not limited to bone marrow transplant, intravenous injection, in situ injection or transplant. In a preferred embodiment, the MUC1* targeting CAR is administered to a person diagnosed with or at risk of developing a MUC1-positive cancer.

There are many possible anti-MUC1* CAR constructs that can be transduced into T cells or other immune cells for the treatment or prevention of MUC1* positive cancers. CARs are made up of modules and the identity of some of the modules is relatively unimportant, while the identity of other modules is critically important.

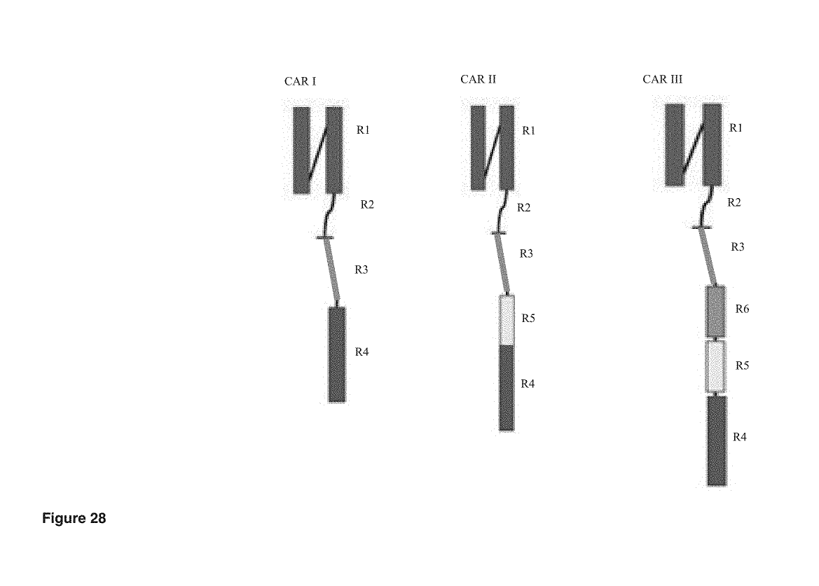

Our experiments demonstrate that the antibody recognition fragment at the outermost portion of the CAR is critically important because it targets the immune cell bearing the CAR to the tumor site. The intracellular signaling motifs are also very important but can be interchanged. FIG. 28 shows a schematic of the components of CAR and the various sequences that may be included in a CAR. Referring to FIG. 28,

R1 is: nothing; or

a ligand or a fragment of a ligand of a cancer associated antigen; or

a ligand or a fragment of a ligand of MUC1 or MUC1*; or

an antibody or antibody fragment wherein the antibody or antibody fragment binds to MUC1 or MUC1*; or an antibody or antibody fragment wherein the antibody or antibody fragment binds to PSMGFR*, wherein the antibody may be human or humanized; or an antibody or antibody fragment of MN-E6, MN-C2, MN-C3 or MN-C8 or humanized MN-E6, MN-C2, MN-C3 or MN-C8; or a single chain variable fragment of an antibody, scFv, that binds to a cleaved MUC1 or MUC1*; or a scFv of MN-E6, MN-C2, MN-C3 or MN-C8, which may be humanized; or a peptide that binds to MUC1* or PSMGFR peptide; or is an antibody fragment, a scFv, or a peptide that binds the PSMGFR portion of MUC1*; or is comprised of sequence from the humanized variable region of MN-E6 (SEQ ID NOS: 38-39 and 93-94), MN-C2 (SEQ ID NOS: 144-145 and 194-195), MN-C3 (SEQ ID NOS: 439-440 and 486-487) and MN-C8 (SEQ ID NOS: 525-526 and 565-566). In one aspect, R1 is a scFv that binds the PSMGFR portion of MUC1* comprised of sequence from humanized MN-E6 scFv (SEQ ID NOS: 232-237), humanized MN-C2 scFv (SEQ ID NOS: 238-243), humanized MN-C3 scFv (SEQ ID NOS: 244-249) or humanized MN-C8 scFv (SEQ ID NOS: 250-255). In another aspect, R1 is a scFv that binds the PSMGFR portion of MUC1* comprised of sequence from humanized MN-E6 scFv (SEQ ID NOS: 232-237) or humanized MN-C2 scFv (SEQ ID NOS: 238-243). In one example R1 is a scFv that binds the PSMGFR portion of MUC1* comprised of sequence from humanized MN-E6 scFv (SEQ ID NOS: 232-237)

R2 is a polypeptide flexible linker that connects the recognition portion to the transmembrane domain of the CAR. In one aspect, R2 can be a polypeptide linker of different length from 5 to 250 amino acids. In another aspect, R2 is a polypeptide linker of human origin. In one aspect, R2 can be made of or a modification of the Fc region of a human immunoglobulin (IgG, IgA, IgE, IgM or IgD). I another aspect, R2 can be the hinge region or a modification of the hinge region of a human immunoglobulin (IgG, IgA, IgE, IgM or IgD). In one aspect, R2 can be the hinge region or a modification of the hinge region of a T-cell receptor (CD8a, CD28 or CD4). In one example, R2 is the hinge region of CD8a, the hinge region of human IgD or the Fc domain of human IgG1.

R3 is a transmembrane domain. In one aspect, R3 can be a transmembrane domain or a modification of a transmembrane domain of any transmembrane human proteins. In another aspect, R3 can be a transmembrane domain or a modification of a transmembrane domain from human cell receptor. In one aspect, R3 can be a transmembrane domain or a modification of a transmembrane domain of a T-cell receptor (CD8a, CD4, CD28, CD3z, OX40 or 41-BB). In another aspect, R3 is a transmembrane domain from the first cytoplasmic co-stimulatory domain of the CAR. In one aspect, R3 can be a transmembrane domain or a modification of a transmembrane domain of a T-cell receptor extended with 1, 2, 3, 4 or 5 amino acids of the cytoplasmic domain associated to the transmembrane domain. In another aspect, R3 can be a transmembrane domain or a modification of a transmembrane domain of a T-cell receptor extended with 1, 2, 3, 4 or five amino acids of the cytoplasmic domain associated to the transmembrane domain followed by a cystein for disulfide bond formation. In one example, R3 is the transmembrane domain of CD8a or CD4.

R4 is a signaling domain from a T-cell receptor. In one aspect, R4 can be the cytoplasmic signaling domain of CD3-zeta, CD27, CD28, 4-1BB, OX40, CD30, CD40, ICAm-1, LFA-1, ICOS, CD2, CD5, CD7 and Fc receptor gamma domain. In one example, R4 is the cytoplasmic domain of CD3-zeta. Several examples of humanized CAR with single signaling domain (CAR I) were regenerated: CAR MN-E6 CD3z (SEQ ID NOS: 294-295); CAR MN-C2 CD3z (SEQ ID NOS: 606-607)

R5 is a co-stimulatory domain from a T-cell receptor. In one aspect, R5 can be the cytoplasmic signaling domain of CD27, CD28, 4-1BB, OX40, CD30, CD40, ICAm-1, LFA-1, ICOS, CD2, CD5, CD7 and Fc receptor gamma domain. R5 will be different from R4 and R6. In one example, R5 is the cytoplasmic domain of CD28, 4-1BB or OX40. Several examples of humanized CAR with two signaling domain (CAR II) were regenerated: CAR MN-E6 CD28/CD3z (SEQ ID NOS: 297-298); CAR MN-E6 4-1BB/CD3z (SEQ ID NOS: 300-301); CAR MN-E6 OX40/CD3z (SEQ ID NOS: 616-617); CAR MN-C2 CD28/CD3z (SEQ ID NOS: 608-609); CAR MN-C2 4-1BB/CD3z (SEQ ID NOS: 610-611); CAR MN-C2 OX40/CD3z (SEQ ID NOS: 612-613); MN-C3 4-1BB/CD3z (SEQ ID NOS: 600-601); CAR MN-E6-Fc/8/41BB/CD3z (SEQ ID NOS:310-311); CAR MN-E6 FcH/8/41BB/CD3z (SEQ ID NOS:315-316); CAR MN-E6 Fc/4/41BB/CD3z (SEQ ID NOS:318-319); CAR MN-E6 FcH/4/41BB/CD3z (SEQ ID NOS:321-322); CAR MN-E6 IgD/8/41BB/CD3z (SEQ ID NOS:323-324); CAR MN-E6 IgD/4/41BB/CD3z (SEQ ID NOS:327-328); CAR MN-E6 X4/8/41BB/CD3z (SEQ ID NOS:330-331); CAR MN-E6 X4/4/41BB/CD3z (SEQ ID NOS:333-334); CAR MN-E6 8+4/4/41BB/CD3z (SEQ ID NOS:336-337).

R6 is a co-stimulatory domain from a T-cell receptor. In one aspect, R6 can be the cytoplasmic signaling domain of CD27, CD28, 4-1BB, OX40, CD30, CD40, ICAm-1, LFA-1, ICOS, CD2, CD5, CD7 and Fc receptor gamma domain. R6 will be different from R4 and R5. In one example, R5 is the cytoplasmic domain of CD28. Several examples of humanized CAR with two signaling domain (CAR III) were regenerated: CAR MN-E6 CD28/OX40/CD3z (SEQ ID NOS: 618-619); CAR MN-E6 CD28/4-1BB/CD3z (SEQ ID NOS: 303-304); CAR MN-C2 CD28/4-1BB/CD3z (SEQ ID NOS: 306-307); CAR MN-C2 CD28/OX40/CD3z (SEQ ID NOS: 614-615)

We and others have shown that intracellular signaling modules, such as CD3-zeta (SEQ ID NOS: 373-376), CD28 (SEQ ID NOS: 377-378) and 41BB (SEQ ID NOS: 379-380), alone or in combinations stimulate immune cell expansion, cytokine secretion and immune cell mediated killing of the targeted tumor cells (Pule M A, Straathof K C, Dotti G, Heslop H E, Rooney C M and Brenner M K (2005) A chimeric T cell antigen receptor that augments cytokine release and supports clonal expansion of primary human T cells. Mol Ther. 12(5):933-941; Hombach A A, Heiders J, Foppe M, Chmielewski M and Abken H. (2012) OX40 costimulation by a chimeric antigen receptor abrogates CD28 and IL-2 induced IL-10 secretion by redirected CD4(+) T cells. Oncoimmunology. 1(4):458-466; Kowolik C M, Topp M S, Gonzalez S, Pfeiffer T, Olivares S, Gonzalez N, Smith D D, Forman S J, Jensen M C and Cooper L J. (2006) CD28 costimulation provided through a CD19-specific chimeric antigen receptor enhances in vivo persistence and antitumor efficacy of adoptively transferred T cells. Cancer Res. 66(22):10995-11004; Loskog A, Giandomenico V, Rossig C, Pule M, Dotti G and Brenner M K. (2006) Addition of the CD28 signaling domain to chimeric T-cell receptors enhances chimeric T-cell resistance to T regulatory cells. Leukemia. 20(10):1819-1828; Milone M C, Fish J D, Carpenito C, Carroll R G, Binder G K, Teachey D, Samanta M, Lakhal M, Gloss B, Danet-Desnoyers G, Campana D, Riley J L, Grupp S A and June C H. (2009) Chimeric receptors containing CD137 signal transduction domains mediate enhanced survival of T cells and increased antileukemic efficacy in vivo. Mol Ther. 17(8):1453-1464; Song D G, Ye Q, Carpenito C, Poussin M, Wang L P, Ji C, Figini M, June C H, Coukos G, Powell D J Jr. (2011) In vivo persistence, tumor localization, and antitumor activity of CAR-engineered T cells is enhanced by costimulatory signaling through CD137 (4-1BB). Cancer Res. 71(13):4617-4627). Less important is the identity of the short extracellular piece that presents the antibody fragment, the transmembrane domain, and the short cytoplasmic tail that comes before the intracellular signaling motifs.

The identity of the recognition antibody fragment that targets the CAR to a tumor is critically important. For the treatment of MUC1 positive or MUC1* positive cancers, that antibody recognition fragment must bind to the extracellular domain of portion of MUC1 that remains after cleavage and shedding of the bulk of the extracellular domain, which contains the tandem repeat domains. In one aspect of the invention, the portion that remains comprises the PSMGFR sequence. In another aspect of the invention, the portion of MUC1 that remains after cleavage and shedding contains the PSMGFR sequence plus up to nine (9) more amino acids extended at the N-terminus. In another aspect of the invention, the portion of MUC1 that remains after cleavage and shedding contains the PSMGFR sequence plus up to twenty one (21) more amino acids extended at the N-terminus. In one aspect, the antibody recognition fragment binds to at least twelve contiguous amino acids of a PSMGFR peptide. In another aspect of the invention, the antibody recognition fragment binds to a peptide comprising the sequence SNIKFRPGSVVVQLTLAFREGTINVHDVETQFNQYKTEAASRY (SEQ ID NO:620); or SVVVQLTLAFREGTINVHDVETQFNQYKTEAASRY (SEQ ID NO:621).

As a demonstration, a single chain antibody fragment that included the variable domain of the monoclonal anti-MUC1* antibodies called MN-E6 or MN-C2 were engineered into a panel of CARs (Table 1). The MUC1* targeting CARs were then transduced, separately or in combinations, into immune cells. When challenged with surfaces presenting a MUC1* peptide, an antigen presenting cell transfected with MUC1*, or MUC1* positive cancer cells, the immune cells that were transduced with MUC1* targeting CARs elicited immune responses, including cytokine release, killing of the targeted cells and expansion of the immune cells (Table 2).

In one case, human Jurkhat cells were transduced with MUC1*-targeting CARs and upon exposure to a surface presenting the PSMGFR peptide, K562 antigen presenting cells that had been transfected with MUC1* or MUC1* positive cancer cells, the Jurkhat cells secreted IL-2. In another case, purified human T cells were transduced with MUC1*-targeting CARs and upon exposure to a surface presenting the PSMGFR peptide, K562 antigen presenting cells that had been transfected with MUC1* or MUC1* positive cancer cells, the T cells secreted IL-2, interferon gamma, and killed the targeted antigen presenting cells and cancer cells, while the T cells expanded. As demonstrated, CARs that comprise an antibody fragment, wherein the antibody fragment is able to bind to the PSMGFR peptide, a transmembrane domain and a cytoplasmic tail bearing co-stimulatory domains, elicit an immune system anti-tumor cell response when said CARs are transduced into immune cells, which include T cells. Therefore, other antibodies, antibody fragments or antibody mimics that are able to bind to the PSMGFR peptide will perform similarly and can be used to treat or prevent cancers. Those skilled in the art will recognize that there are a number of technologies available for transfecting or transducing cells with CARs and the invention is not limited by the method used for making the immune cell express a MUC1*-targeting CAR.

For example, the gene encoding the CARs and activated T cell induced genes described herein can be virally transduced into an immune cell using viruses, which may or may not result in the CAR gene being integrated into the genome of the recipient cell. Virus delivery systems and viral vectors including but not limited to retroviruses, including gamma-retroviruses, lentivirus, adenoviruses, adeno-associated viruses, baculoviruses, poxvirus, herpes simplex viruses, oncolytic viruses, HF10, T-Vec and the like can be used. In addition to viral transduction, CARs and activated T cell induced genes described herein can be directly spliced into the genome of the recipient cell using methods such as CRISPR technology, CRISPR-Cas9 and -CPF1, TALEN, Sleeping Beauty transposon system, and SB 100×.

Similarly, the identity of molecules that make up the non-targeting portions of the CAR such as the extracellular domain, transmembrane domain and membrane proximal portion of the cytoplasmic domain, are not essential to the function of a MUC1*-targeting CAR. For example, the extracellular domain, transmembrane domain and membrane proximal portion of the cytoplasmic domain can be comprised of portions of CD8, CD4, CD28, or generic antibody domains such as Fc, CH2CH3, or CH3. Further, the non-targeting portions of a CAR can be a composite of portions of one or more of these molecules or other family members.

One aspect of the invention is a method for treating a patient diagnosed with, suspected of having, or at risk of developing a MUC1 positive or MUC1* positive cancer, wherein the patient is administered an effective amount of immune cells that have been transduced with a MUC1* targeting CAR. In another aspect of the invention, the immune cells are T cells isolated from a patient, which are then transduced with CARs wherein the targeting head of the CAR binds to MUC1*, and after expansion of transduced T cells, the CAR T cells are administered in an effective amount to the patient. In yet another aspect of the invention, the immune cells are T cells isolated from a patient, which are then transduced with CARs wherein the targeting head of the CAR comprises portions of huMN-E6, huMN-C2, huMN-C3 or huMN-C8, and after optional expansion of transduced T cells, the CAR T cells are administered in an effective amount to the patient. In yet another aspect of the invention, the CAR that is transduced into the immune cell and administered to the patient diagnosed with a MUC1 or MUC1* positive cancer is chosen from the list of CARs in Table 1 or Table 2.

Specifics of CARs Made and Tested

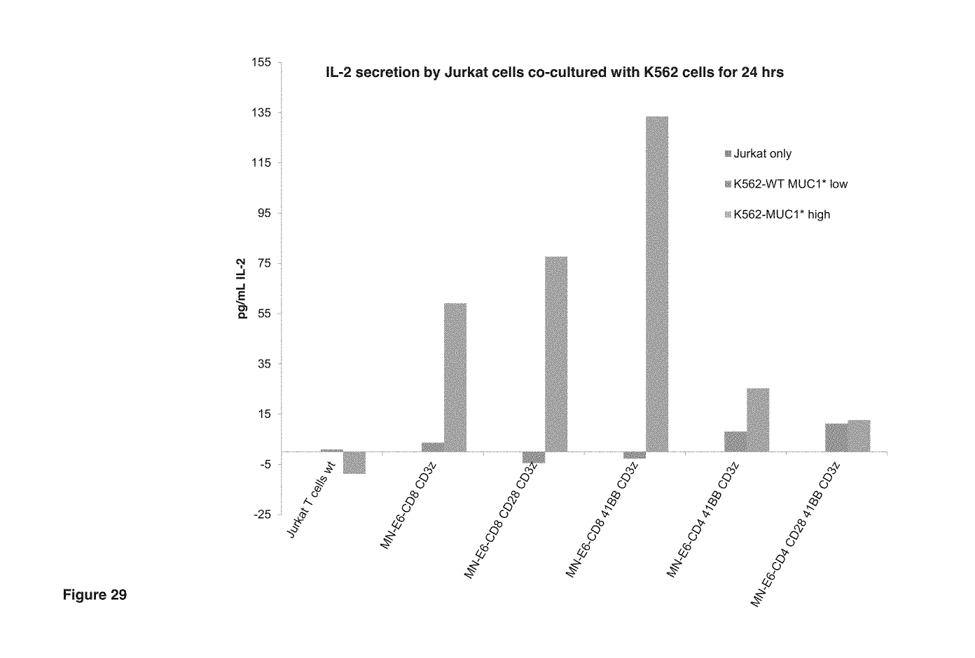

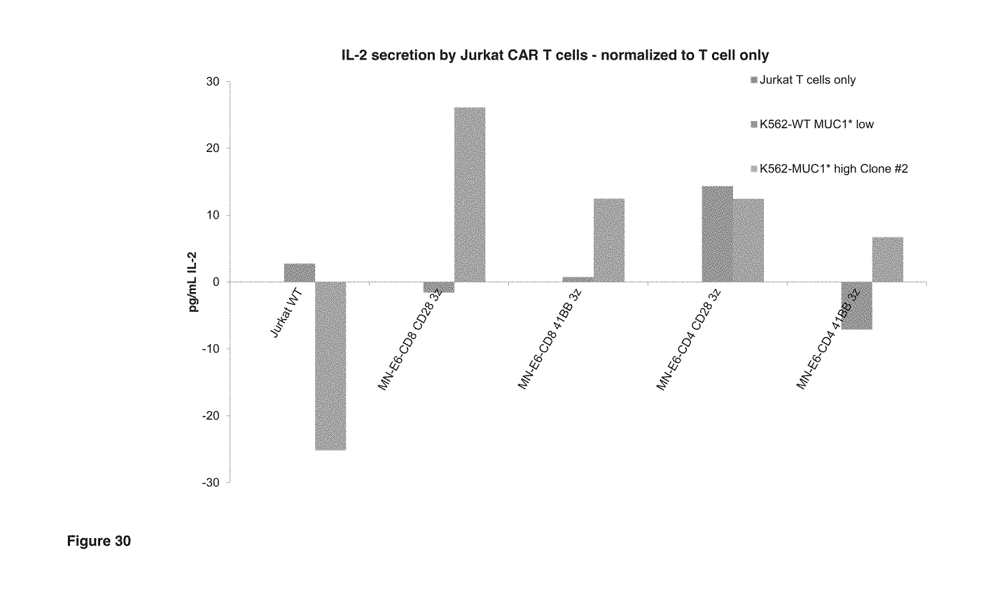

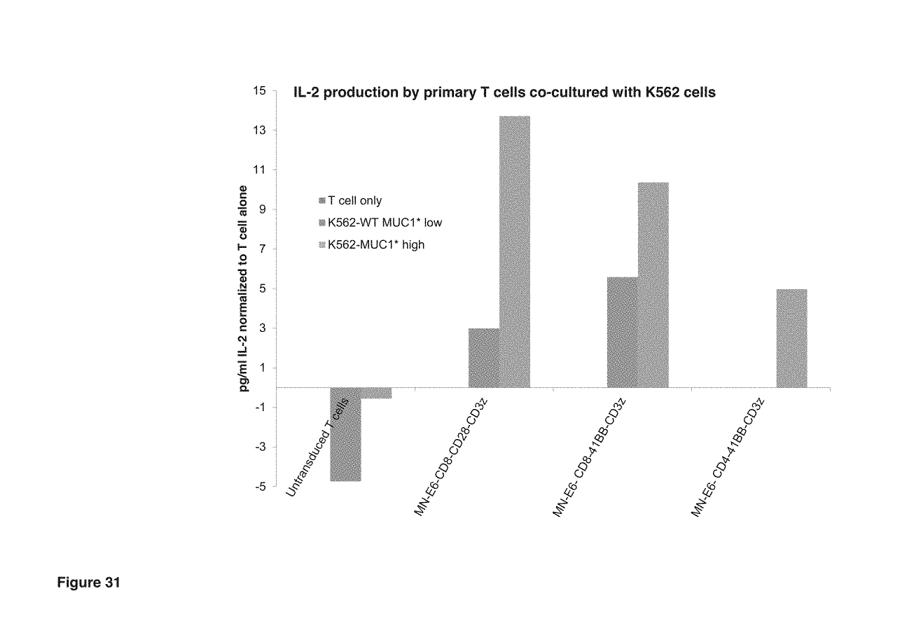

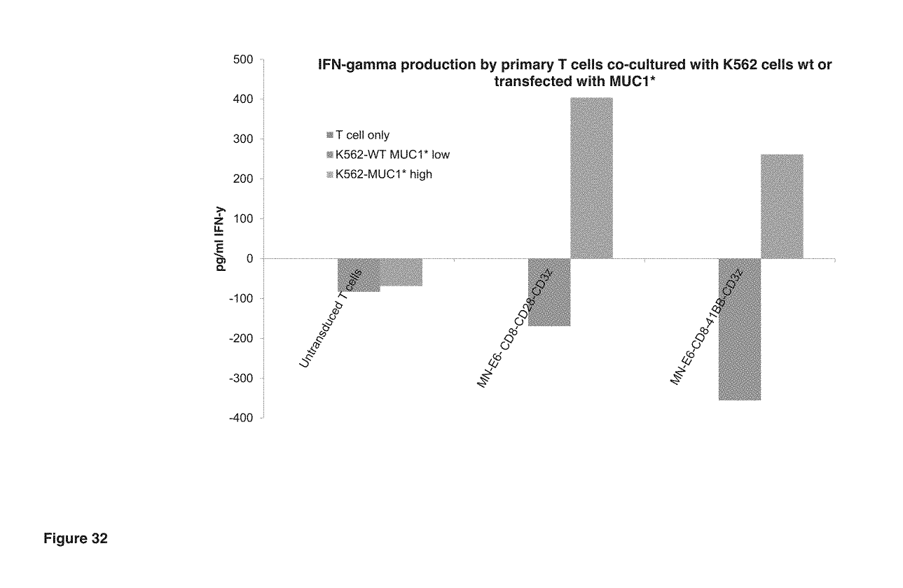

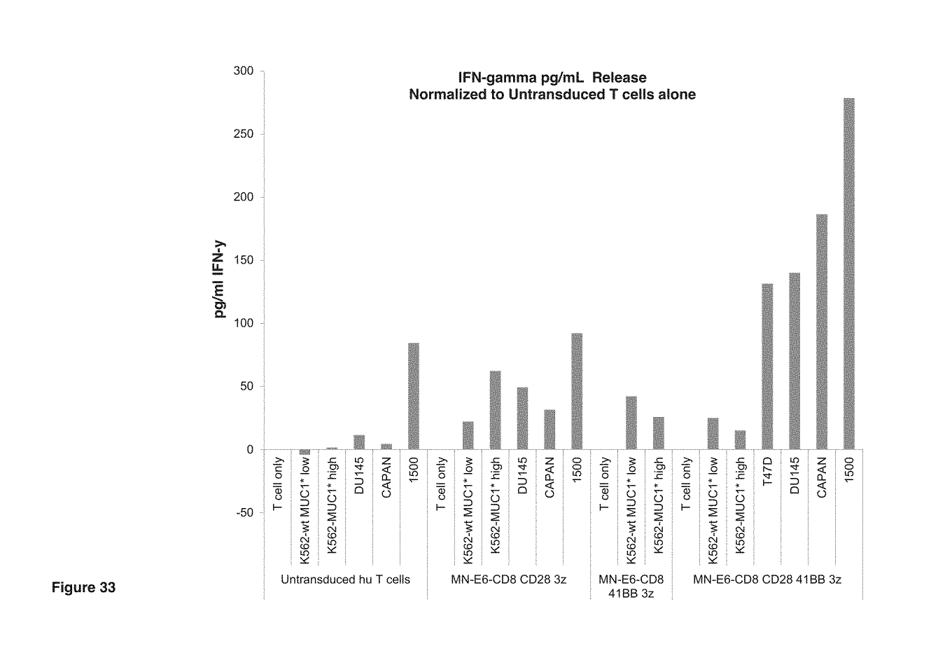

Many MUC1* targeting CARs were generated wherein the targeting antibody fragment at the distal end of the CAR was either MN-E6, MN-C2, MN-C3 or MN-C8. The DNA of each CAR was sequenced to verify that cloning was correctly done. Each construct was then shuffled into an expression plasmid, transfected into cells and then verified that the construct had successfully inserted by Western blot. Surface expression was verified by FACS. The MUC1* targeting CARs were then virally transduced into immune cells. In one aspect, they were transduced into Jurkat cells. In another aspect, they were transduced into primary human T cells that were purified from blood. A series of functional assays were performed and verified that the CARs were functional. Functional assays showed that both Jurkat cells and primary T cells transduced with MUC1* targeting CAR secreted the cytokine IL-2 and interferon gamma (IFN-g) when challenged with cells or surfaces presenting MUC1*. Table 1 lists the CARs that were made and tested. Table 2 lists cytokine release data for some of the CARs after transduction into human T cells and co-culture with a variety of cancer cells. FIG. 29 is a graph of an experiment measuring IL-2 cytokine secretion by Jurkat cells that were transduced with a panel of CARs, including MN-E6 CD8/CD3z, MN-E6 CD8/CD28/CD3z, MN-E6 CD8/41BB/CD3z, MN-E6 CD4/CD28/CD3z and MN-E6 CD4/CD28/41BB/CD3z. IL-2 was secreted only when the CAR Jurkat cells were exposed to K562-wt cells or K562 cells that had been transfected with MUC1*. It should be noted that the parent K562-wt cells express very low levels of MUC1*. Another group of CARs transfected into Jurkat cells was similarly tested for cytokine secretion. FIG. 30 shows IL-2 secretion by Jurkat T cells that were transduced with MN-E6 CD8/CD28/CD3z, MN-E6 CD8/41BB/CD3z, MN-E6 CD4/CD28/CD3z or MN-E6 CD4/41BB/CD3z, when the CAR T cells were exposed to K562-wt cells or K562 cells that had been transfected with MUC1*. Similarly, FIG. 31 shows IL-2 cytokine secretion by primary human T cells that were transduced with MN-E6 CD8/CD28/CD3z, MN-E6 CD8/41BB/CD3z or MN-E6 CD4/41BB/CD3z. Cytokine secretion only occurred when the MUC1* targeting CAR T cells were exposed to K562-wt cells or K562 cells that had been transfected with MUC1*. Another cytokine that is secreted by activated T cells when they see a target cell is interferon-gamma (IFN-g). FIG. 32 shows that interferon-gamma was secreted by primary human T cells that were transduced with a panel of CARs, including MN-E6 CD8/CD28/CD3z and MN-E6 CD4/41BB/CD3z, when the CAR T cells were exposed to K562-wt cells or K562 cells that had been transfected with MUC1*. Interferon-gamma was similarly secreted by primary human T cells that were transduced with a panel of CARs, including MN-E6 CD8/CD28/CD3z, MN-E6 CD8/41BB/CD3z and MN-E6 CD8/CD28/41BB/CD3z, when the MUC1* targeting CAR T cells were exposed to K562-wt cells, K562 cells that had been transfected with MUC1*, or MUC1* positive cancer cells of prostate cancer (DU145), breast cancer (1500) or pancreatic cancer (Capan) (FIG. 33).

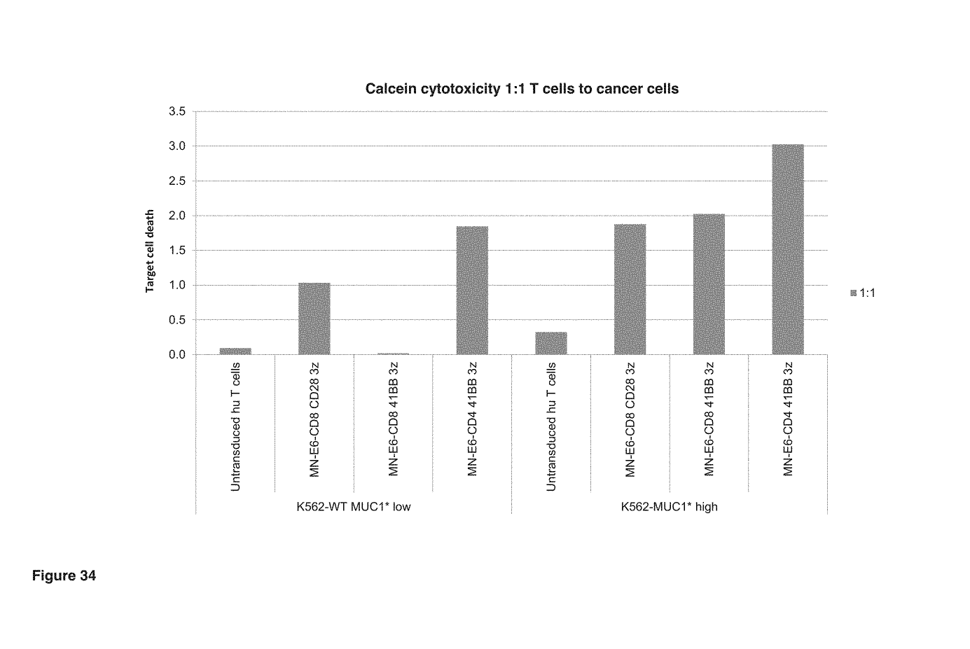

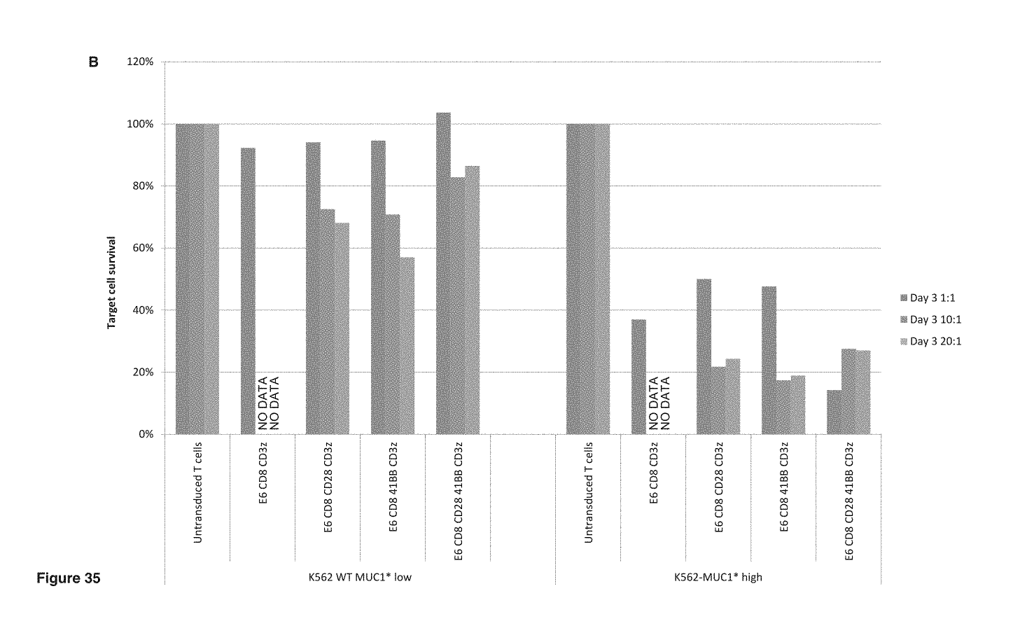

Another measure of function of CAR T cells is whether or not they induce killing of the targeted cells. T cells transfected with a variety of CARs comprising antibody fragments that bind to the PSMGFR sequence of MUC1* killed MUC1* expressing cells in co-culture assays. In one assay, target MUC1* expressing cells are incubated with calcein. When they are mixed with CAR T cells wherein the CAR comprises an antibody fragment such as MN-E6, MN-C2, MN-C3 or MN-C8 the CAR T cells kill the MUC1* presenting cells which causes the target cells to lyse and releases calcein into the supernatant. FIG. 34 is a graph of an experiment measuring target cell death when primary human T cells, isolated from a blood sample, that were transduced with a panel of CARs, including MN-E6 CD8/CD28/CD3z, MN-E6 CD8/41BB/CD3z and MN-E6 CD4/41BB/CD3z, when the CAR T cells were exposed to K562-wt cells or K562 cells that had been transfected with MUC1*. The ratio of T cells to target cells was 1:1 and the cells were co-cultured for 24 hours. FIGS. 35A-35B are graphs of FACS measuring a time course of target cell survival from Day 1 to Day 3. Primary human T cells, isolated from a blood sample, were transduced with a panel of CARs, including humanized MN-E6-CD8-3z, MN-E6-CD8-CD28-3z, MN-E6-CD8-41BB-3z and MN-E6-CD8-CD28-41BB-3z. The CAR T cells were then exposed to K562-wt cells that naturally express low levels of MUC1*, or K562 cells that had been transfected with MUC1* high. The ratio of MUC1* targeting CAR T cells to target cells was either 1:1, 10:1, or 20:1. Surviving cells were detected and measured at Day 1 or Day 3.

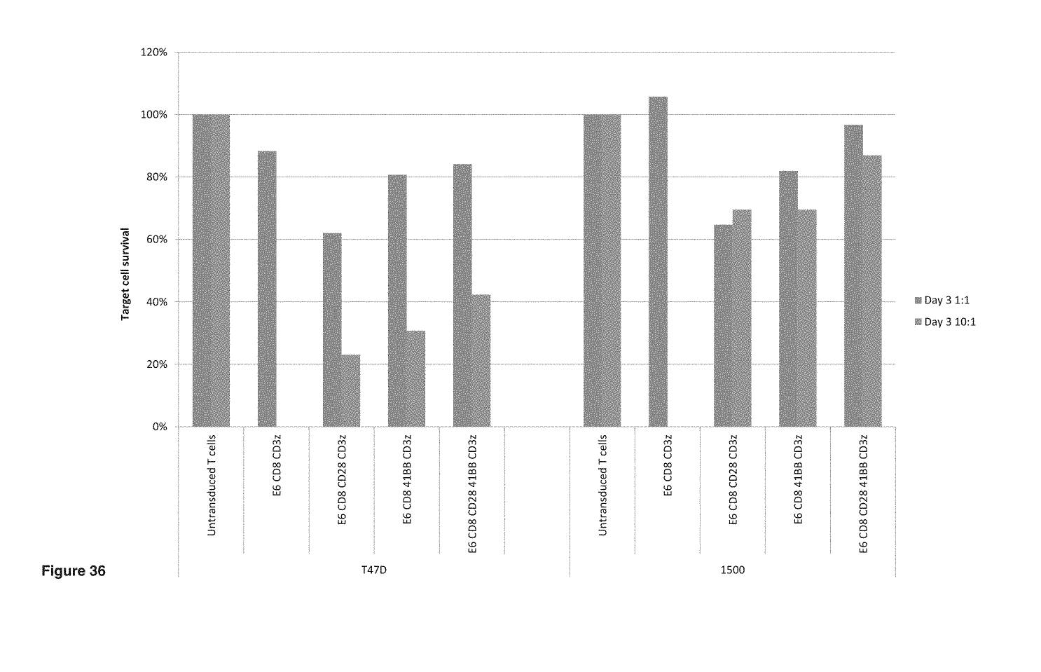

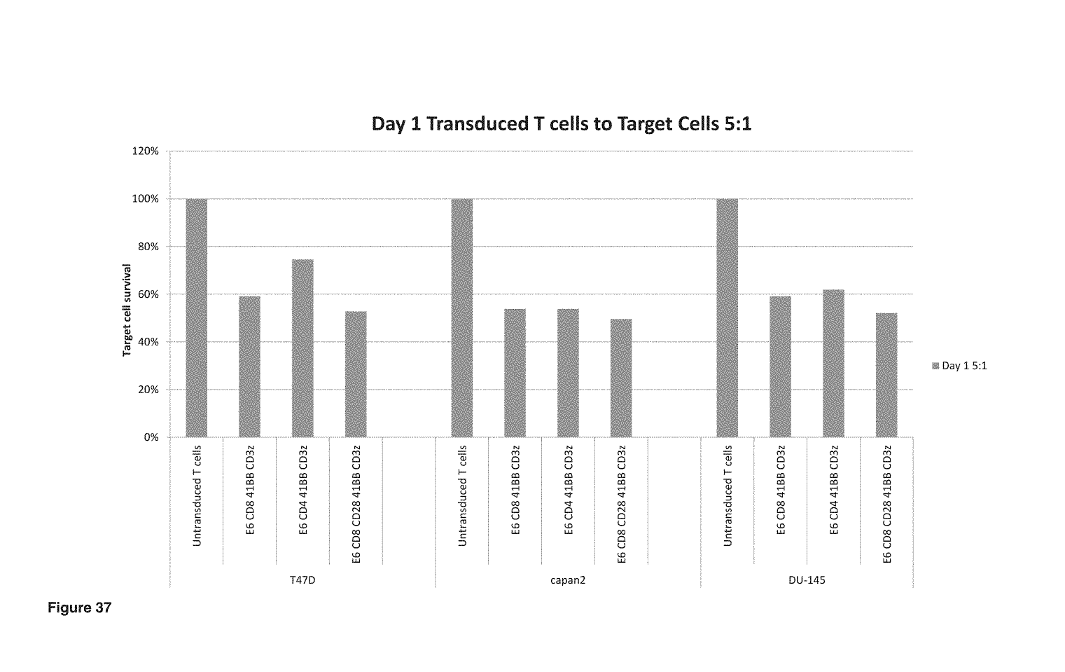

FIG. 36 is a graph of FACS measurements of target cell survival at Day 3 of co-culture experiment. Primary human T cells were transduced with a panel of CARs, including humanized MN-E6-CD8-3z, MN-E6-CD8-CD28-3z, MN-E6-CD8-41BB-3z and MN-E6-CD8-CD28-41BB-3z. The CAR T cells were then exposed to MUC1* positive T47D breast cancer cells or MUC1* positive 1500 aka ZR-75-1 breast cancer cells. The ratio of MUC1* targeting CAR T cells to target cells was either 1:1 or 10:1. As can be seen from the graph, T cells transduced with a MUC1* targeting CAR have a much greater killing effect on MUC1* cancer cells than the untransduced control T cells. In addition, the killing effect is much greater when the ratio of T cells:target cells is increased. FIG. 37 is a graph of FACS measurements of target cell survival at Day 1 of co-culture experiment. Primary human T cells were transduced with a panel of CARs, including humanized MN-E6-CD8-41BB-3z, MN-E6-CD4-41BB-3z, and MN-E6-CD8-CD28-41BB-3z. The CAR T cells were then exposed to the following MUC1* positive cancer cells: T47D breast cancer; capan2 pancreatic cancer; or DU-145 prostate cancer. The ratio of MUC1* targeting CAR T cells to target cells was 5:1. As can be seen from the graph, T cells transduced with a MUC1* targeting CAR have a much greater killing effect on MUC1* cancer cells than the untransduced control T cells. Note that the measurements were taken after 24 hours with only a 5:1 T cell to target cell ratio. Also note that MUC1* targeting CARs that have a CD4 extracellular domain-transmembrane-cytoplasmic tail work equally well as CD8 constructs.

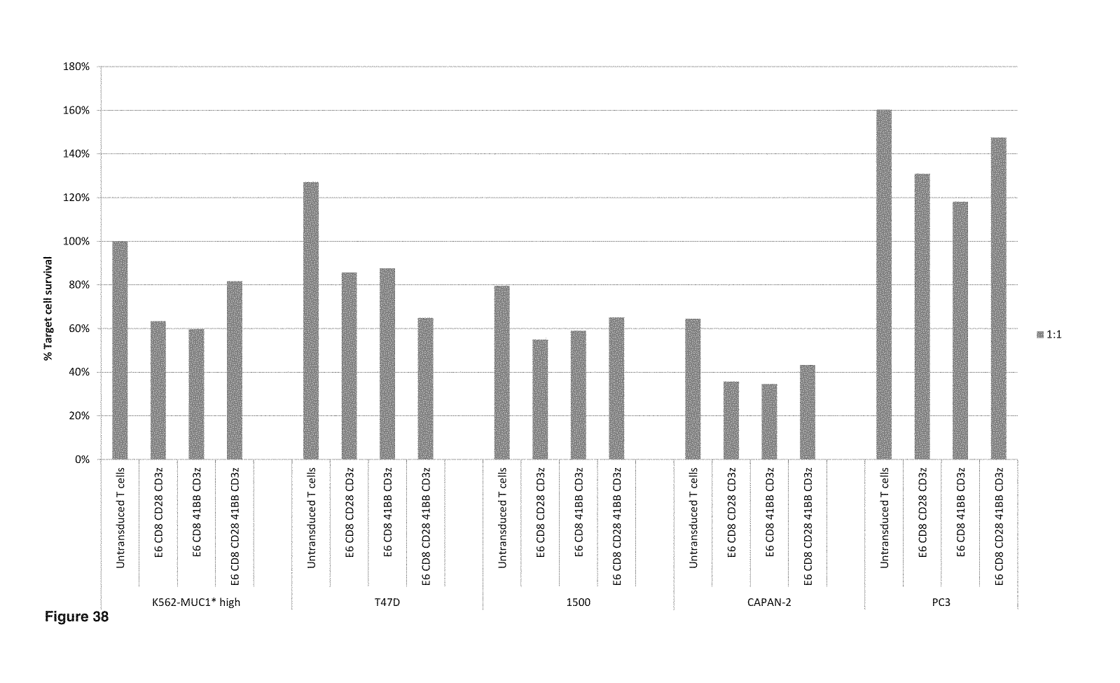

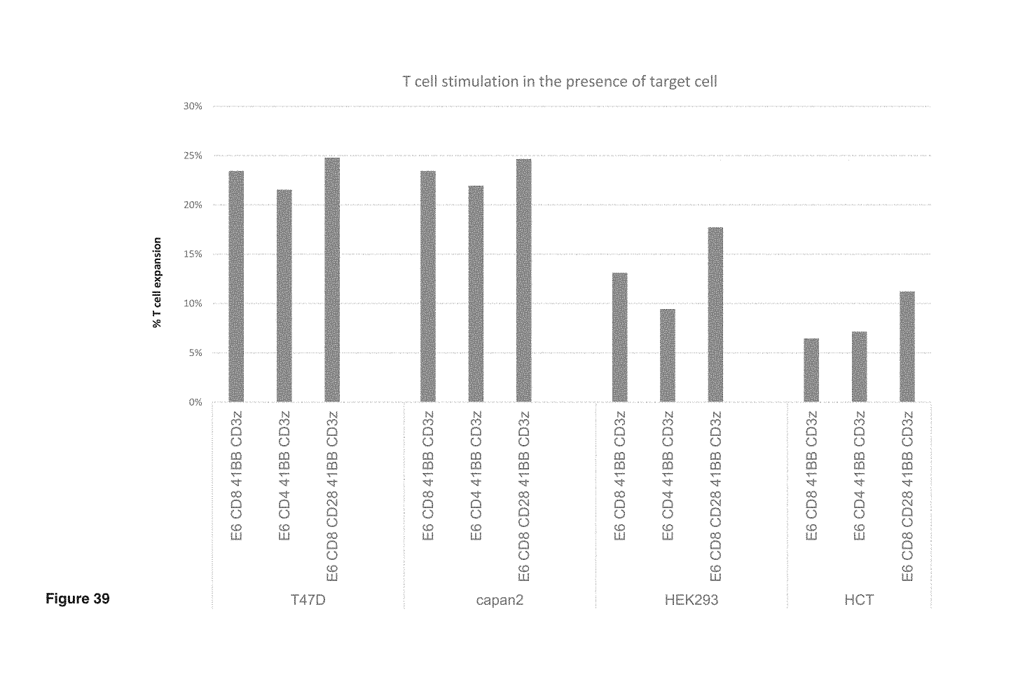

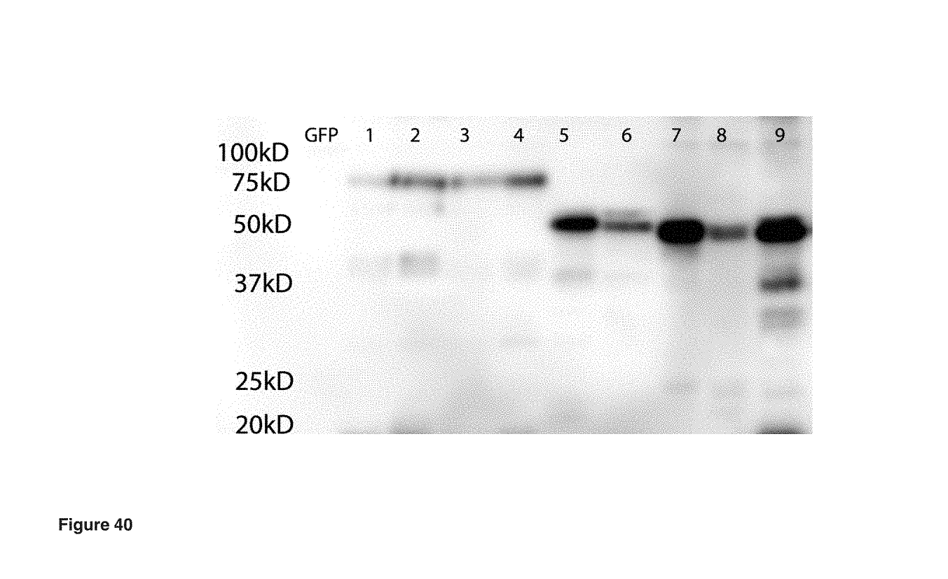

FIG. 38 is a graph of FACS measurements of target cell survival at Day 3 of co-culture experiment. Primary human T cells were transduced with a panel of CARs, including humanized MN-E6-CD8-41BB-3z, MN-E6-CD4-41BB-3z, and MN-E6-CD8-CD28-41BB-3z. The CAR T cells were then exposed to the following MUC1* positive cancer cells: K562 leukemia cells transfected with MUC1*; T47D breast cancer; 1500 aka ZR-75-1 breast cancer cells; or CAPAN-2 pancreatic cancer cells. In addition to the untransduced T cell controls, the assay was performed on PC3 MUC1* negative prostate cancer cells. The ratio of MUC1* targeting CAR T cells to target cells was 1:1. As can be seen from the graph, T cells transduced with a MUC1* targeting CAR have a much greater killing effect on MUC1* cancer cells than the untransduced control T cells. In addition, the killing effect is specific for MUC1* positive cells. Note that MUC1* targeting CARs that have a CD4 extracellular domain-transmembrane-cytoplasmic tail work equally well as CD8 constructs. FIG. 39 is a graph of FACS measurements of CAR T cell expansion over 24 hours in co-culture with target cells at a ratio of 5:1 CAR T cells to target cells. The primary human T cells were transduced with a panel of CARs, including humanized MN-E6-CD8-41BB-3z, MN-E6-CD4-41BB-3z, and MN-E6-CD8-CD28-41BB-3z. The CAR T cells were co-cultured with MUC1* positive T47D breast cancer cells, MUC1* positive Capan pancreatic cancer cells, and MUC1-negative cells HCT-116 colon cancer cells and HEK-293 human embryonic kidney cells. As can be seen from the graph, the CAR T population is increased in the presence of MUC1* positive cells. FIG. 40 shows a photograph of a Western blot of MUC1* targeting CARs. From 1 to 9 are: 1. MN-E6scFv-Fc-8-41BB-CD3z (Human Fc as hinge region with CD8 TM); 2: MN-E6scFv-FcH-8-41BB-CD3z (Human Fc hingeless as hinge region with CD8 TM); 3: MN-E6scFv-Fc-4-41BB-CD3z (Human Fc as hinge region with CD4 TM); 4: MN-E6scFv-FcH-4-41BB-CD3z (Human Fc as hingeless hinge region with CD4 TM); 5: MN-E6scFv-IgD-8-41BB-CD3z (hinge region from human IgD with CD8 TM); 6: MN-E6scFv-IgD-4-41BB-CD3z (hinge region from human IgD with CD4 TM); 7: MN-E6scFv-X4-8-41BB-CD3z (Long flexible linker as hinge region with CD8 TM); 8: MN-E6scFv-X4-4-41BB-CD3z (Long flexible linker as hinge region with CD4 TM); 9: MN-E6scFv-8-4-41BB-CD3z (Hinge region from CD8 and CD4 a with CD4 TM).

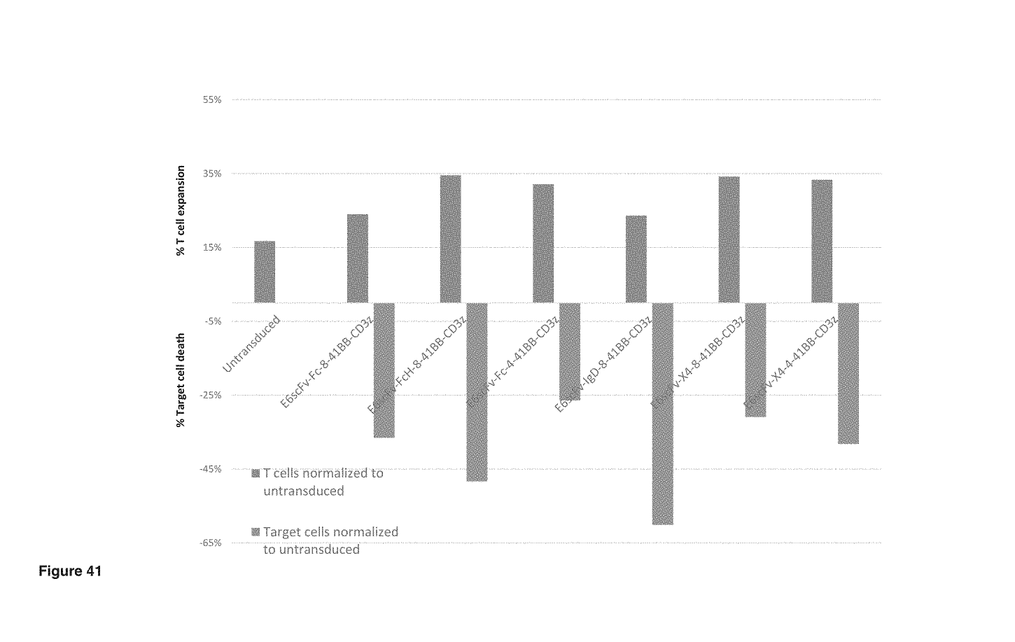

FIG. 41 shows graphs of FACS scans of T47D breast cancer cells co-cultured with human T cells that were transduced with MN-E6scFv-Fc-8-41BB-CD3z, MN-E6scFv-FcH-8-41BB-CD3z (hingeless), MN-E6scFv-Fc-4-41BB-CD3z, MN-E6scFv-IgD-8-41BB-CD3z, MN-E6scFv-X4-8-41BB-CD3z and MN-E6scFv-X4-4-41BB-CD3z. T cells and cancer cells were co-cultured at a 1:1 ratio for 48 hours. T cell counts were normalized to an average of all untransduced T cells and target cells were normalized to each specific cell type when co-cultured with untransduced T cells. The graph shows that when the CAR T cells are co-cultured with MUC1* positive cancer cells, the T cell population expands and the targeted cancer cell population decreases.

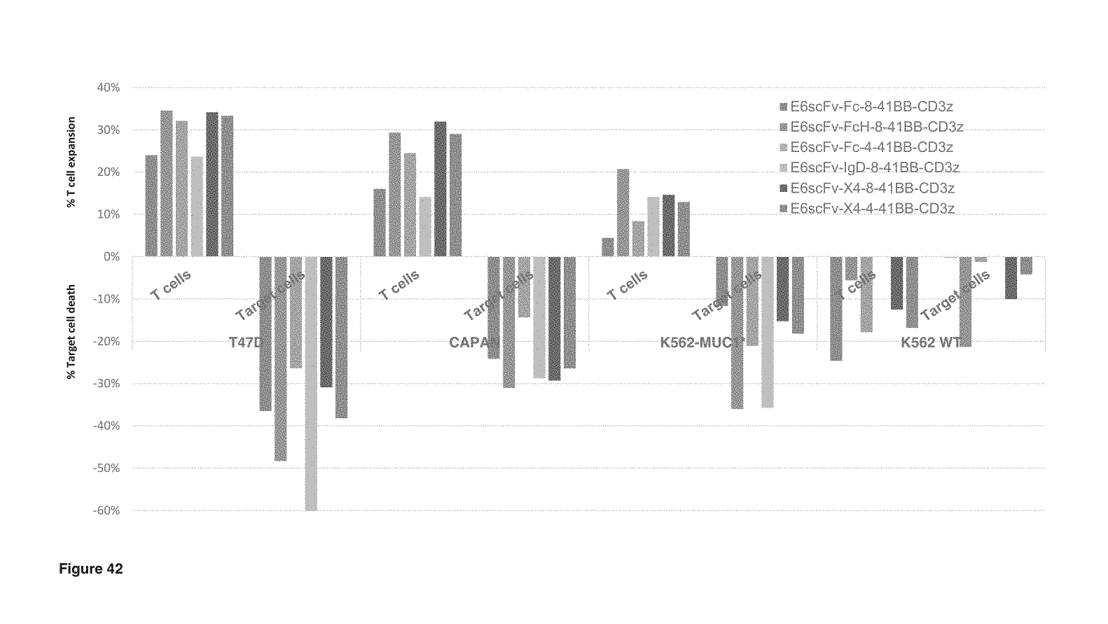

FIG. 42 shows graphs of FACS scans of T47D breast cancer cells, Capan-2 pancreatic cancer cells, K562-MUC1* transfected cells, and K562-wt cells that were co-cultured with human T cells that were transduced with MN-E6scFv-Fc-8-41BB-CD3z, MN-E6scFv-FcH-8-41BB-CD3z, MN-E6scFv-Fc-4-41 BB-CD3z, MN-E6scFv-IgD-8-41BB-CD3z, MN-E6scFv-X4-8-41BB-CD3z and MN-E6scFv-X4-4-41BB-CD3z. T cells and cancer cells were co-cultured at a 1:1 ratio for 48 hours. T cell counts were normalized to an average of all untransduced T cells and target cells were normalized to each specific cell type when co-cultured with untransduced T cells. The graph shows that when the CAR T cells are co-cultured with MUC1* positive cancer cells, the T cell population expands and the targeted cancer cell population decreases.

Specificity of Anti-MUC1* Targeting Antibodies

As these experiments demonstrate, the critical portion of a CAR is the antibody fragment that directs the immune cell to the tumor cell. As we will show in the following section, MN-E6 and MN-C2 are specific for the form of MUC1* that is expressed on tumor cells. The next most important part of a CAR is the cytoplasmic tail bearing immune system co-stimulatory domains. The identity of these domains modulates the degree of immune response but does not affect the specificity. As shown, the identity of the transmembrane portion of a CAR is the least important. It appears that as long as the transmembrane portion has some flexibility and is long enough to allow the antibody fragment to reach its cognate receptor on the tumor cell, it will suffice. This is demonstrated in FIGS. 40-42. CARs comprising the MN-E6 targeting antibody fragment, and intracellular co-stimulatory domains 41BB and CD3-zeta but having a variety of different extracellular, transmembrane and short cytoplasmic tail all worked in that they specifically killed the targeted cells while stimulating the expansion of the host T cells.

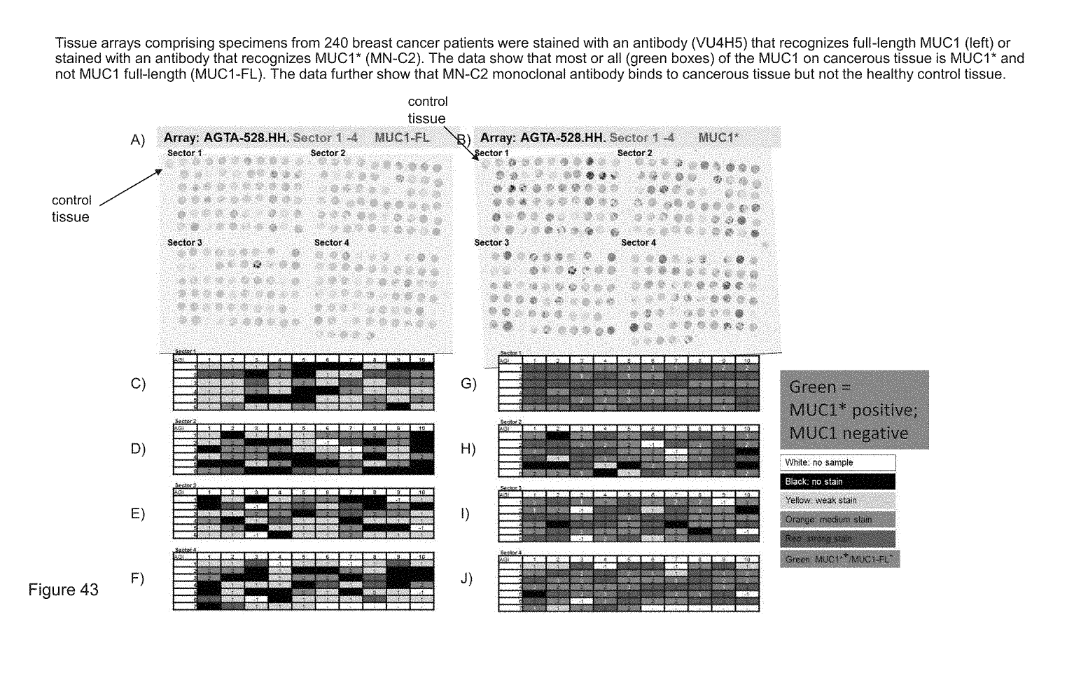

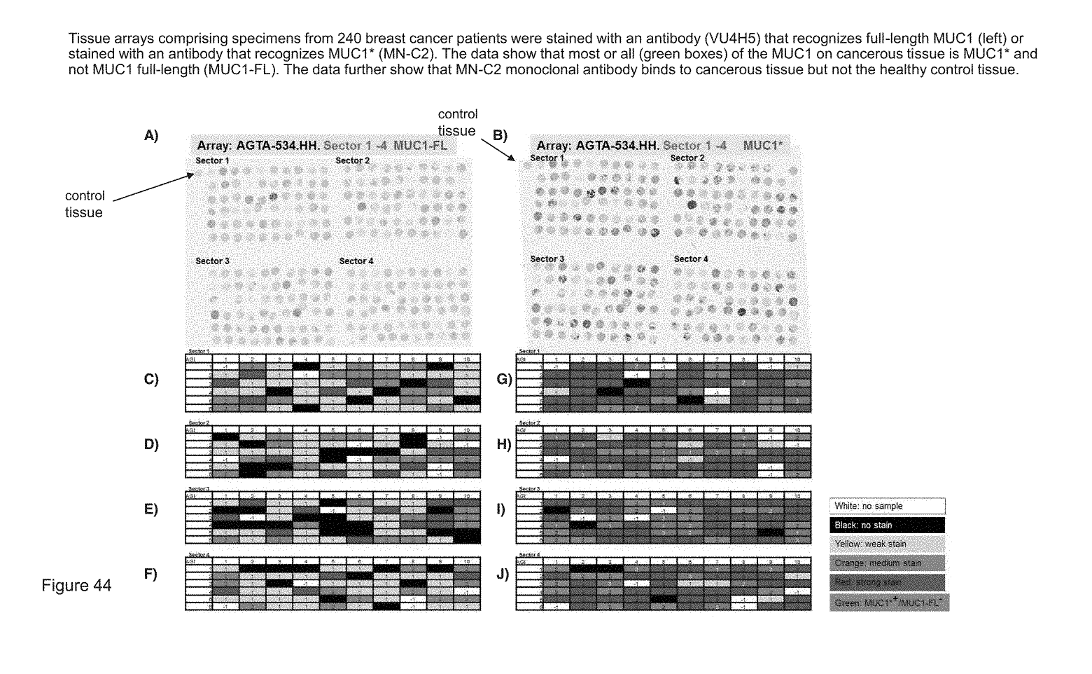

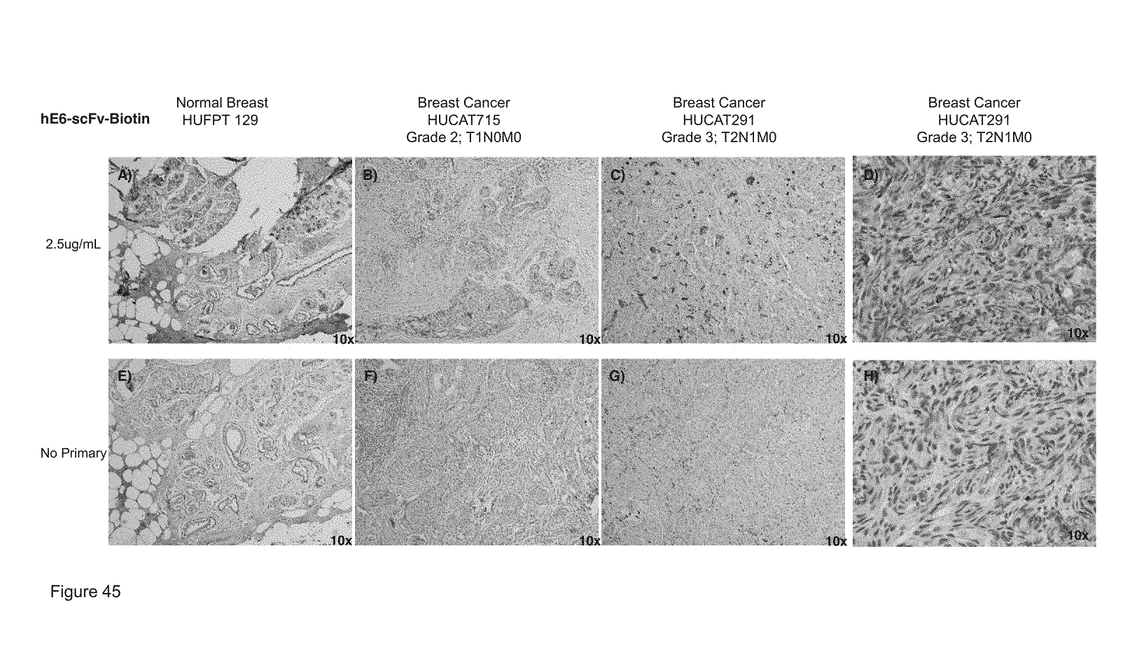

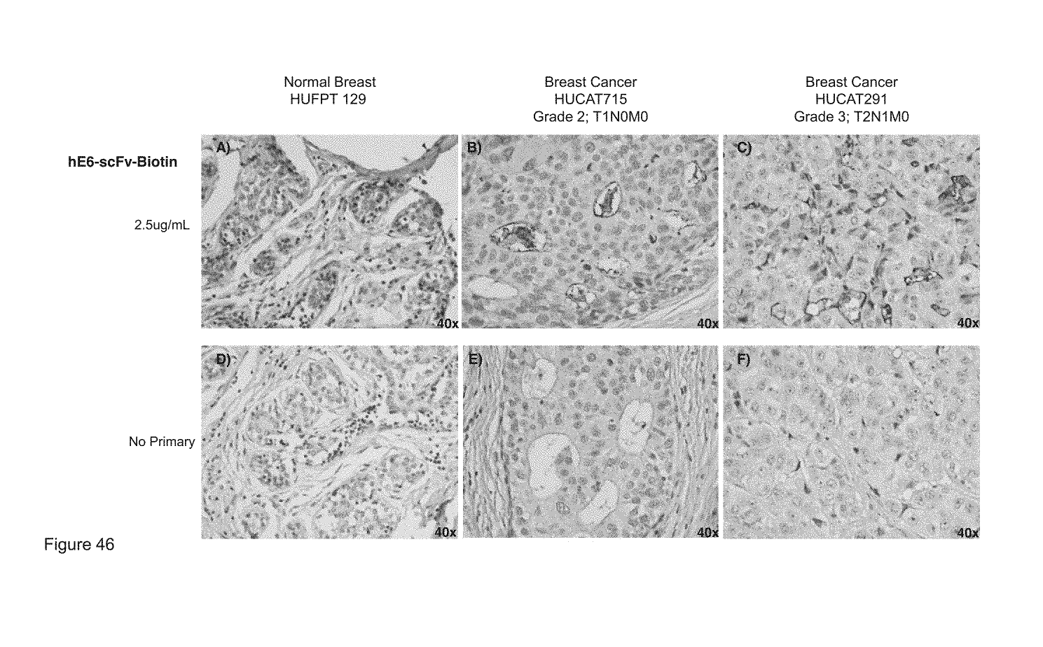

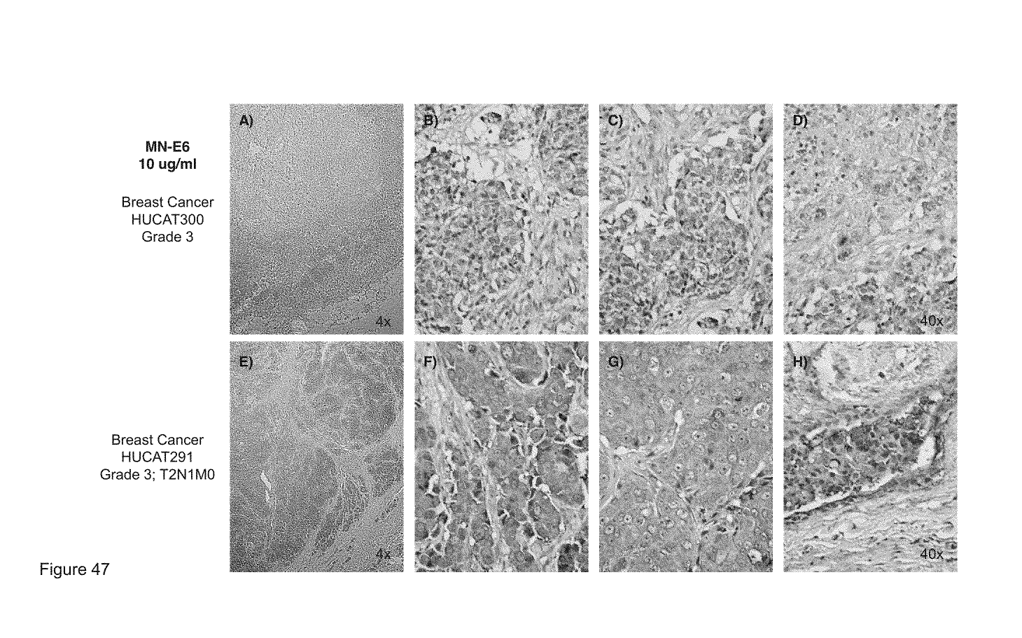

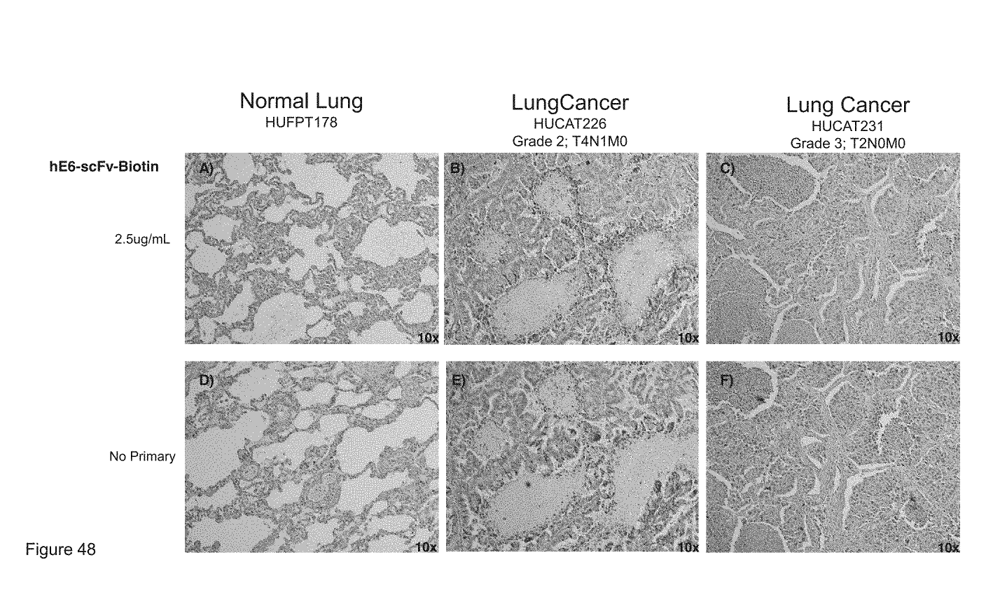

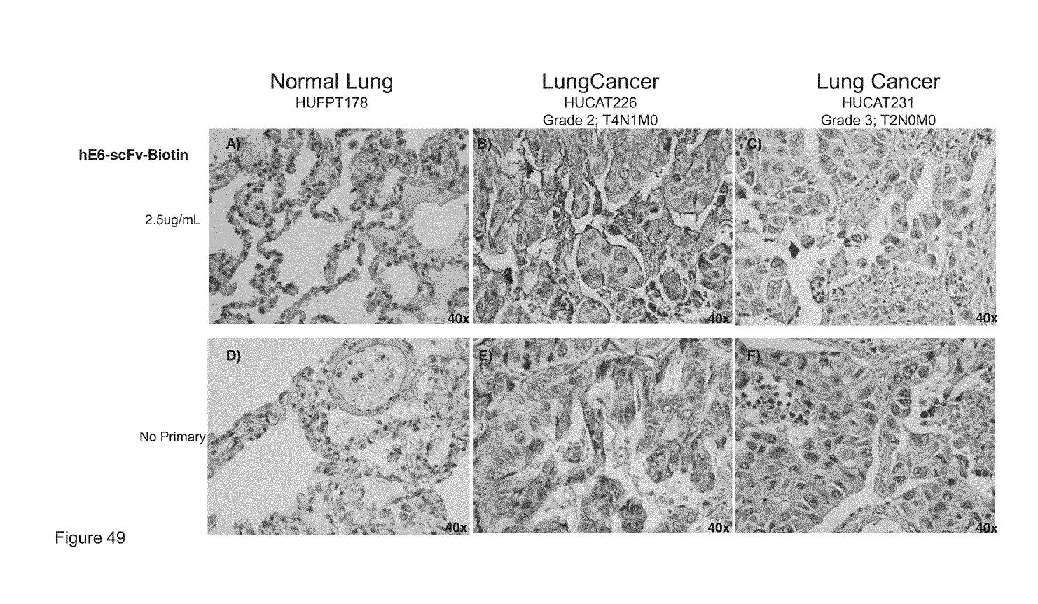

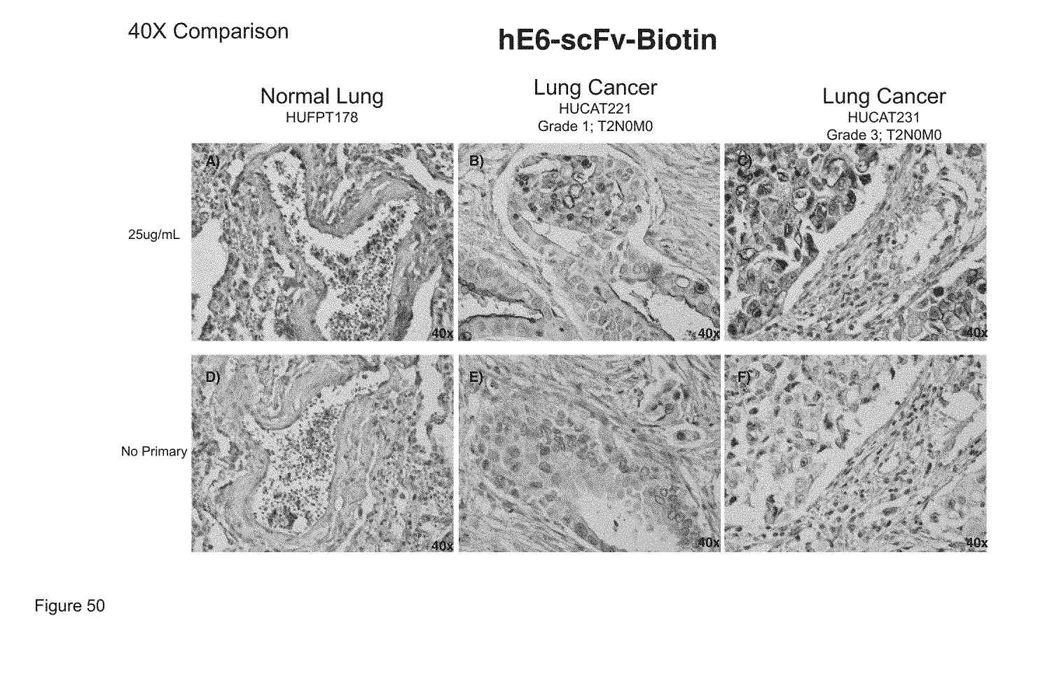

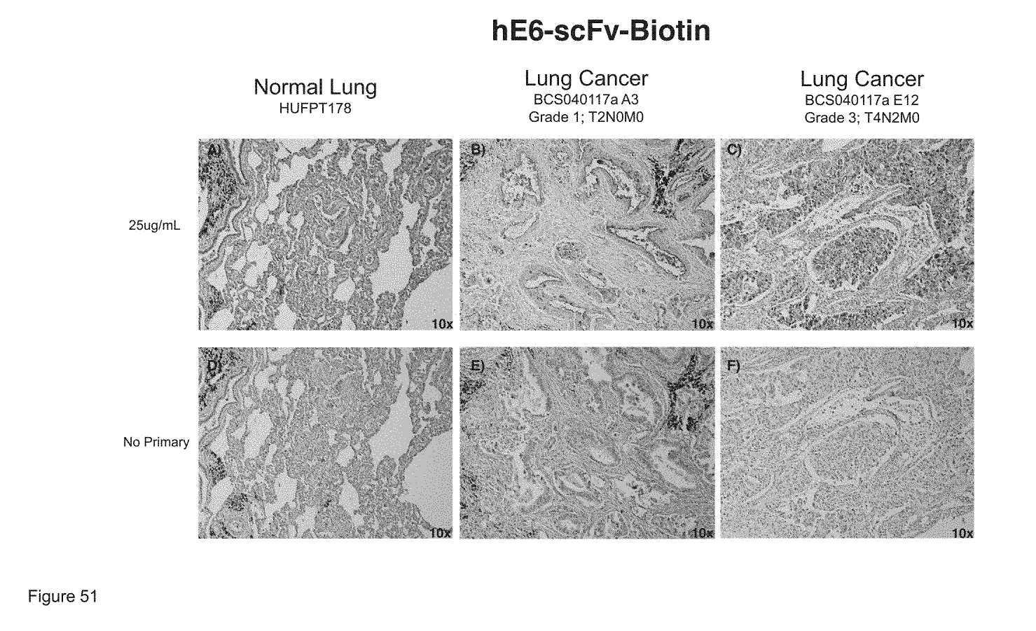

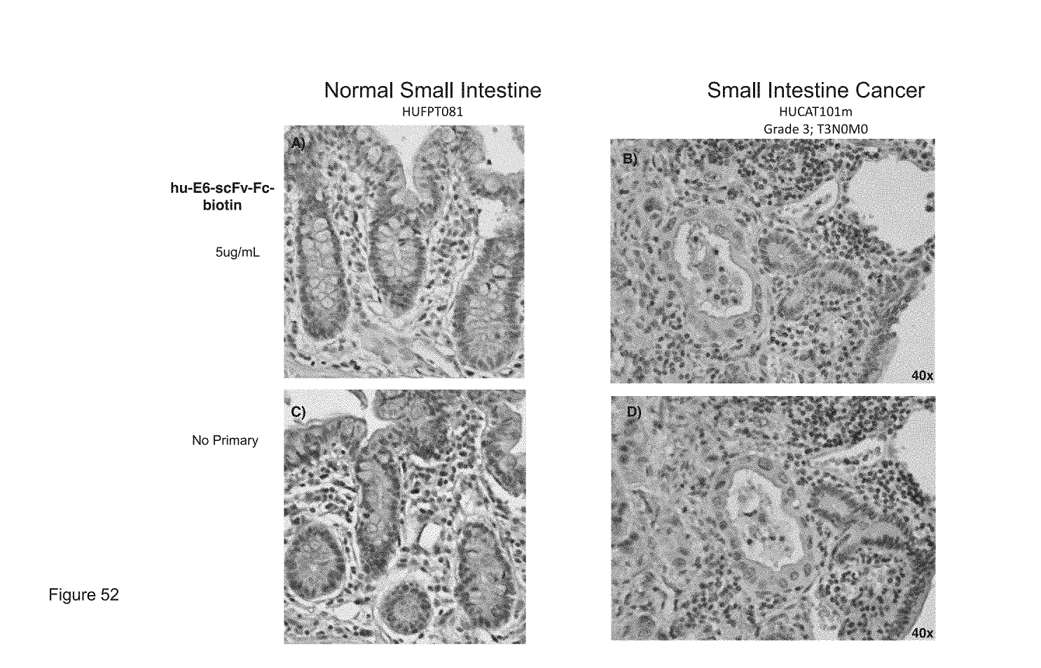

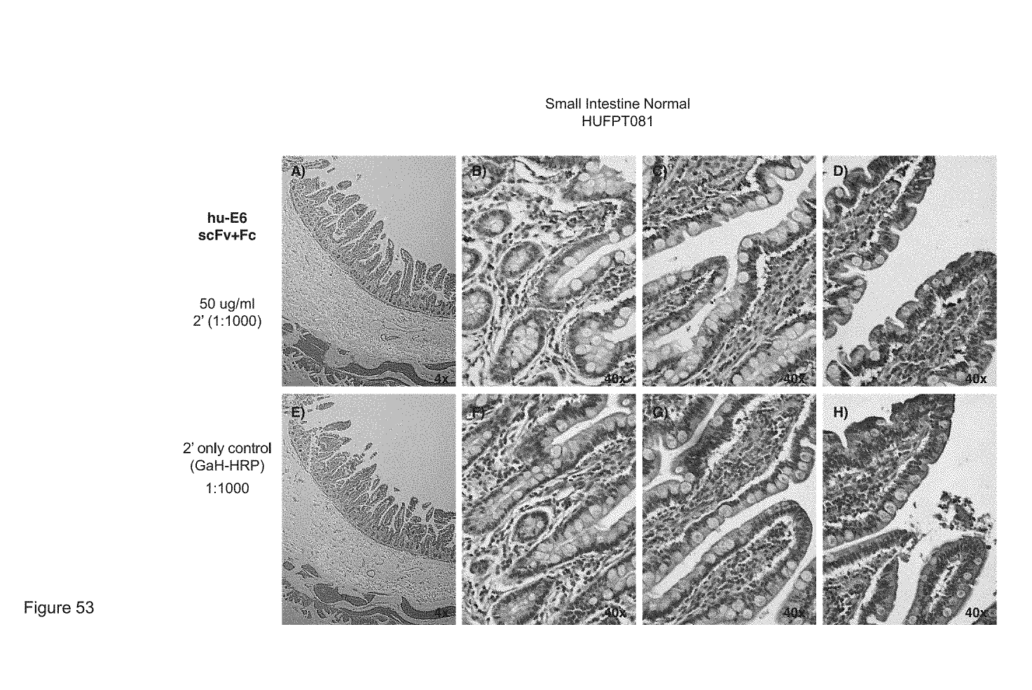

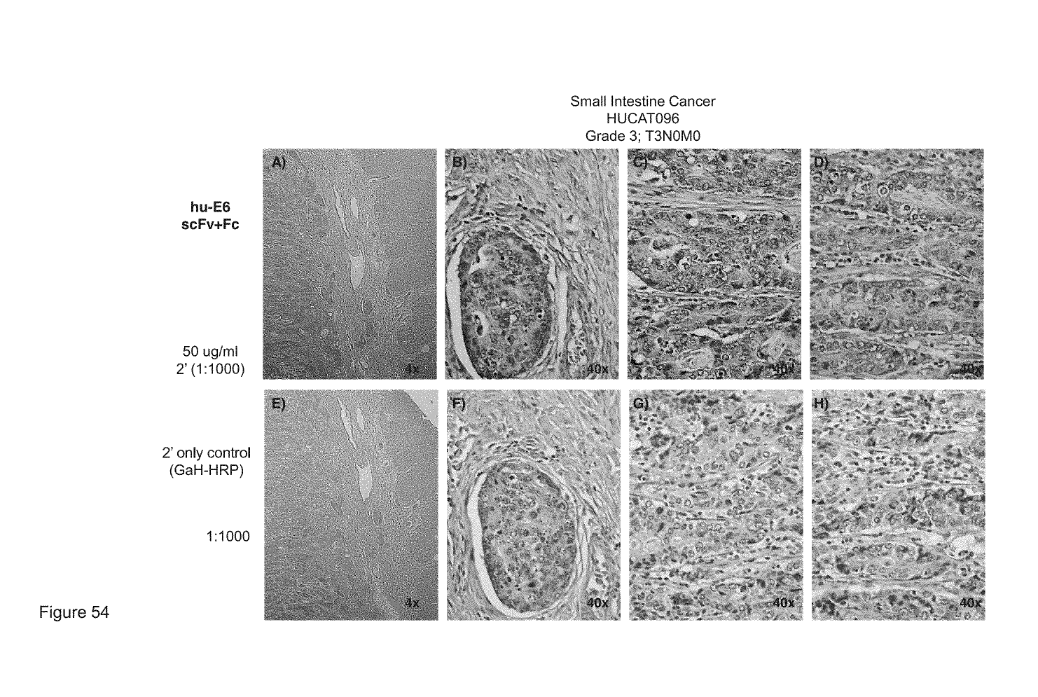

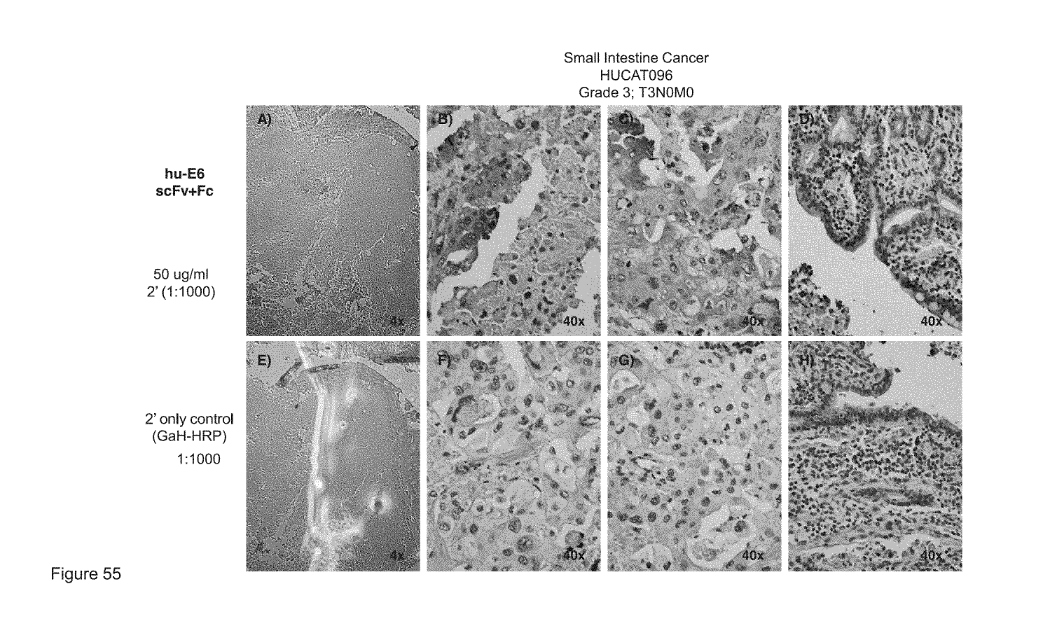

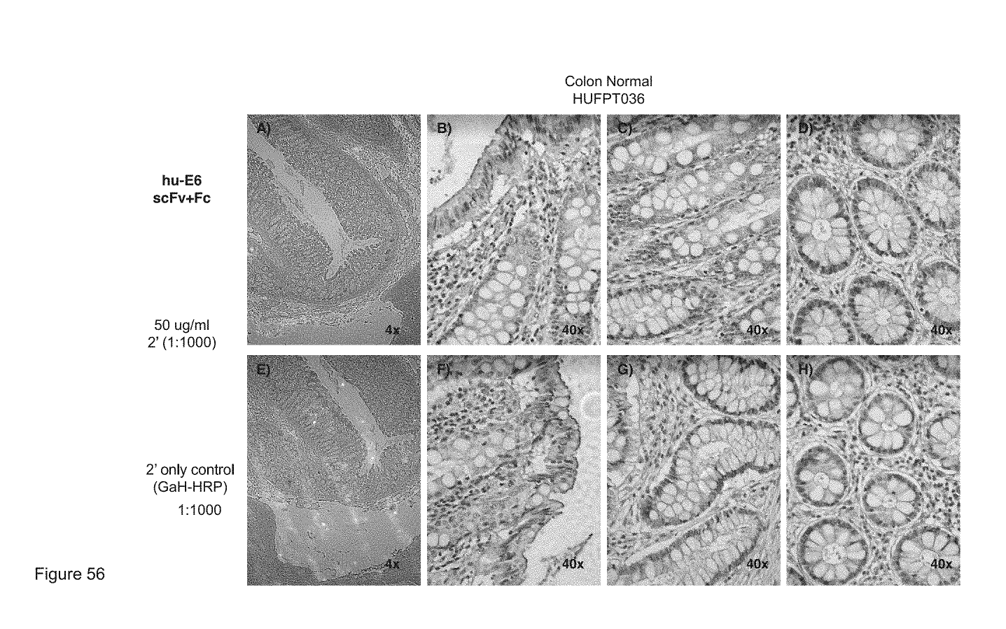

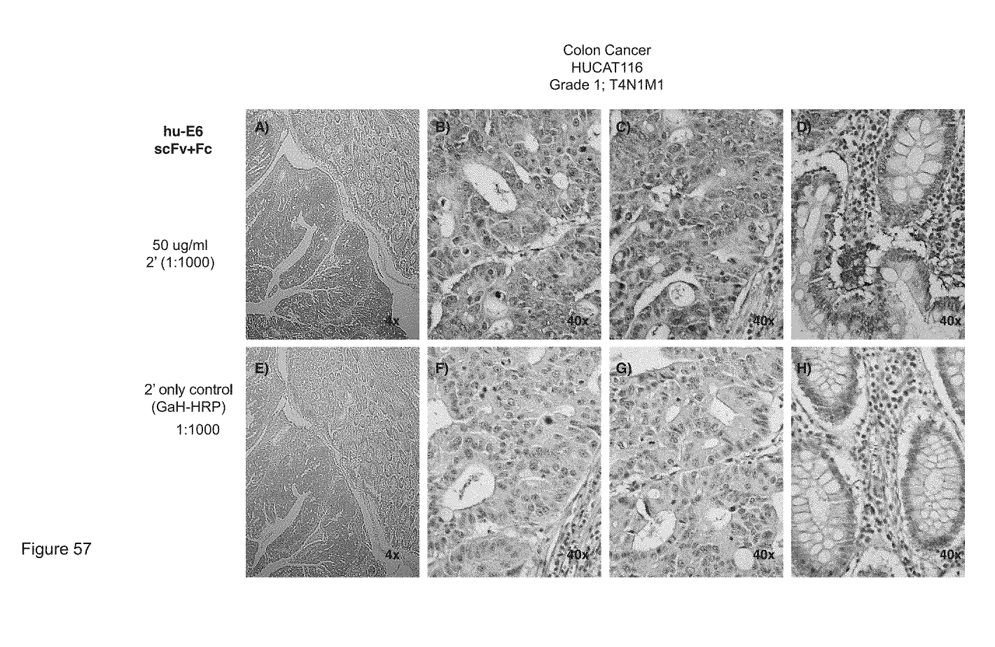

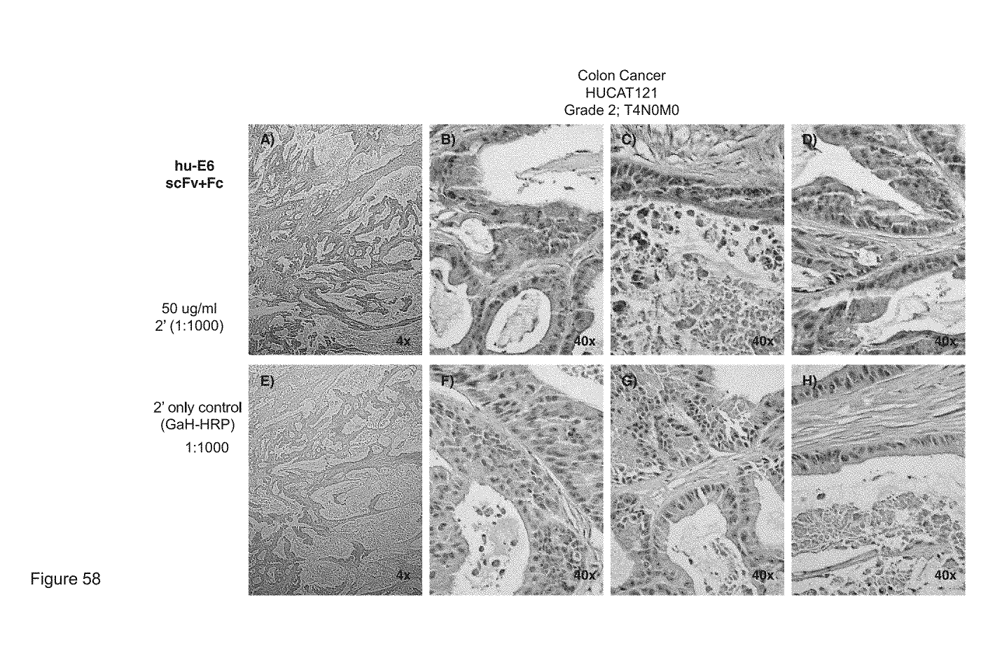

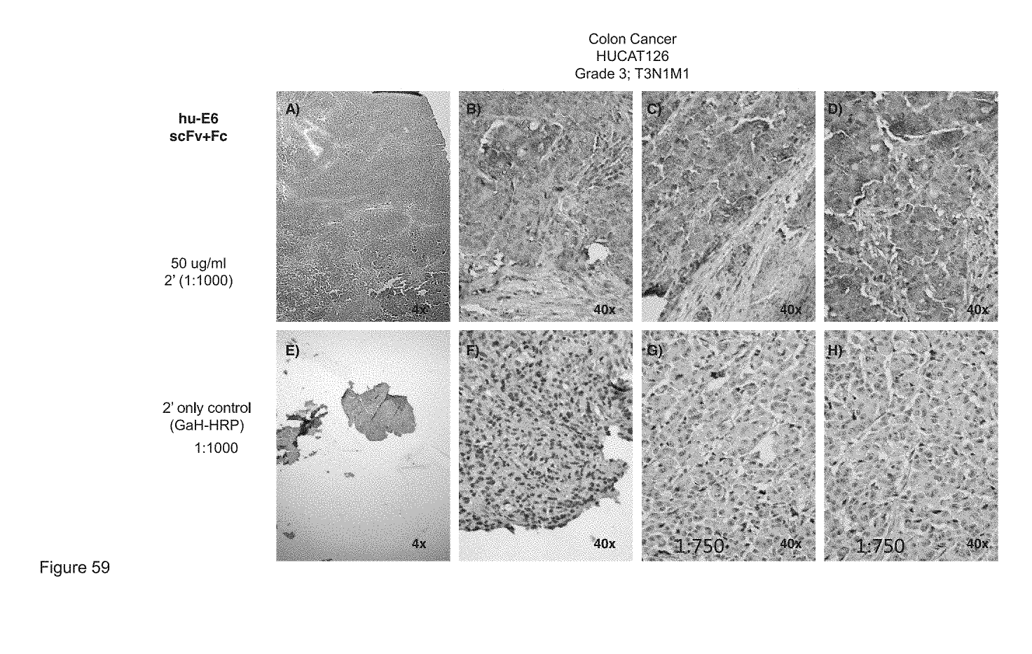

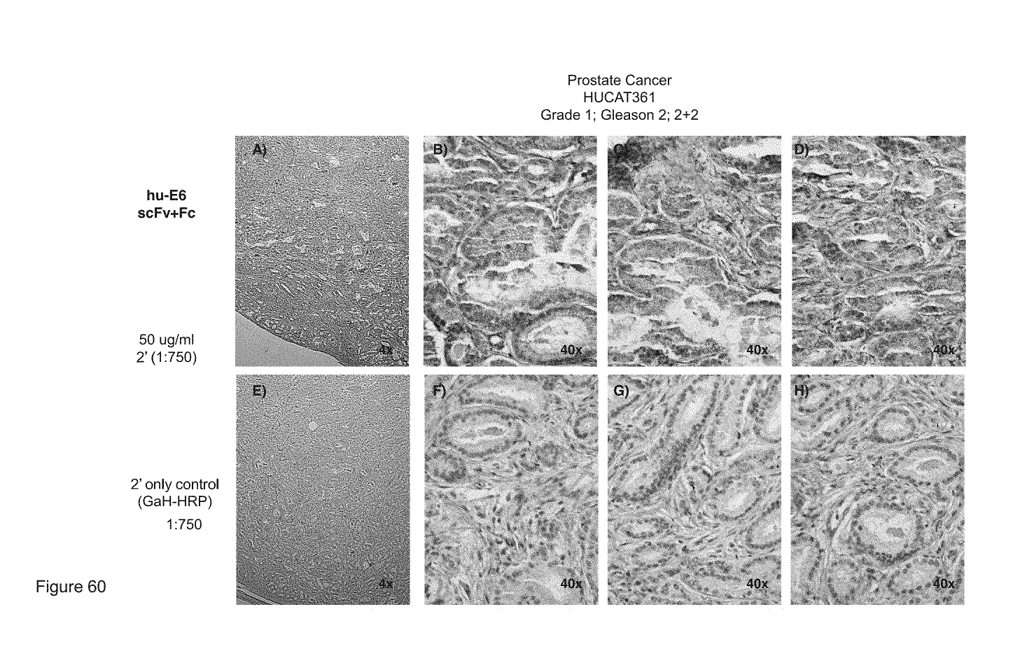

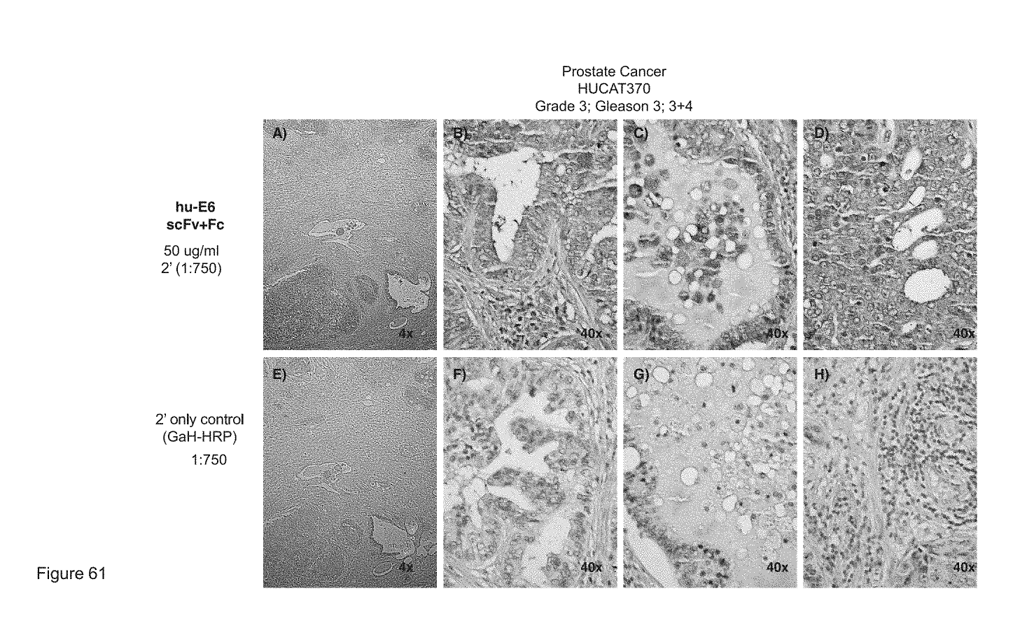

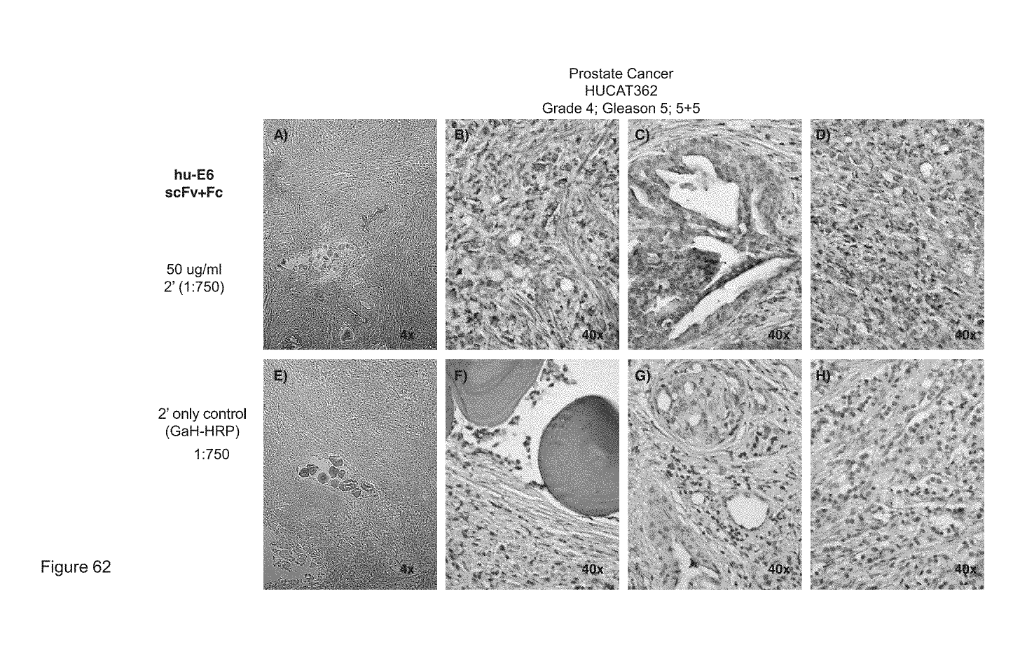

The most accurate way of demonstrating antibody specificity is testing the antibody on normal human tissue specimens compared to cancerous tissue specimens. MN-C2 and MN-E6 were shown to specifically bind to MUC1 or MUC1* positive cancer cells. Several breast tumor arrays were assayed using several anti-MUC1 or MUC1* antibodies. Essentially the studies involving serial sections of breast cancer tissue specimens from over 1,200 different breast cancer patients showed that very little full-length MUC1 remains on breast cancer tissues. The vast majority of the MUC1 expressed is MUC1* and is stained by MN-C2. The analysis was performed by Clarient Diagnostics and tissue staining was scored using the Allred method. For example, FIG. 43 shows serial sections of breast cancer tissue arrays that were stained with either VU4H5, a commercially available anti-MUC1 antibody that binds to the tandem repeats, or MN-C2 that binds to MUC1*. FIGS. 43 and 44 are photographs of breast cancer tissue arrays stained with either VU4H5 which recognizes MUC1-FL (full length) or MN-C2 which recognizes cancerous MUC1*. Tissue staining was scored using Allred scoring method which combines an intensity score and a distribution score. Below the photographs of the tissue arrays are color-coded graphs displaying the results. As can be seen, the arrays stained with VU4H5 are very light and many tissues do not stain at all despite the published reports that MUC1 is aberrantly expressed on over 96% of all breast cancers as evidenced by nucleic acid based diagnostics. In contrast, the arrays stained with MN-C2 are very dark (red versus yellow or white in graph). Additionally, many tissues did not stain at all with anti-full-length MUC1 but stained very dark with MN-C2, (see green boxes in graph). Similarly, we stained normal or cancerous breast tissues with humanized MN-E6 scFv-Fc. The antibody fragment was biotinylated so it could be visualized by a secondary streptavidin based secondary. As can be seen in FIG. 45, hMN-E6 scFv-Fc does not stain normal breast tissue but stains cancerous breast tissue. Further, the intensity and homogeneity of staining increases with tumor grade and/or metastatic grade of the patient (FIGS. 45-46). Similarly, hMN-E6 scFv-Fc did not stain normal lung tissue but did stain lung cancer tissue (FIGS. 47-51) and the intensity and distribution of staining increased as tumor grade or metastatic grade increased. FIG. 52 shows photographs of normal small intestine and cancerous small intestine tissues stained with humanized MN-E6-scFv-Fc biotinylated anti-MUC1* antibody at 5 ug/mL, then stained with a secondary streptavidin HRP antibody. A) is a normal small intestine tissue. B) is small intestine cancer from patient as denoted in the figure. C,D are photographs of the corresponding serial sections that were stained with the secondary antibody alone. FIG. 53 shows photographs of normal small intestine tissues stained with humanized MN-E6-scFv-Fc anti-MUC1* antibody at 50 ug/mL, then stained with a secondary goat-anti-human HRP antibody. A-D are normal small intestine tissue. E-H are photographs of the corresponding serial sections that were stained with the secondary antibody alone. FIG. 54 shows photographs of cancerous small intestine tissues stained with humanized MN-E6-scFv-Fc anti-MUC1* antibody at 50 ug/mL, then stained with a secondary goat-anti-human HRP antibody. A-D are cancerous small intestine tissue from a patient as denoted in figure. E-H are photographs of the corresponding serial sections that were stained with the secondary antibody alone. FIG. 55 shows photographs of cancerous small intestine tissues stained with humanized MN-E6-scFv-Fc anti-MUC1* antibody at 50 ug/mL, then stained with a secondary goat-anti-human HRP antibody. A-D are cancerous small intestine tissue from a patient as denoted in figure. E-H are photographs of the corresponding serial sections that were stained with the secondary antibody alone. FIG. 56 shows photographs of normal colon tissues stained with humanized MN-E6-scFv-Fc anti-MUC1* antibody at 50 ug/mL, then stained with a secondary goat-anti-human HRP antibody. A-D are normal colon. E-H are photographs of the corresponding serial sections that were stained with the secondary antibody alone. FIG. 57 shows photographs of colon cancer tissues stained with humanized MN-E6-scFv-Fc anti-MUC1* antibody at 50 ug/mL, then stained with a secondary goat-anti-human HRP antibody. A-D are colon cancer tissue from a metastatic patient as denoted in figure. E-H are photographs of the corresponding serial sections that were stained with the secondary antibody alone. FIG. 58 shows photographs of colon cancer tissues stained with humanized MN-E6-scFv-Fc anti-MUC1* antibody at 50 ug/mL, then stained with a secondary goat-anti-human HRP antibody. A-D are colon cancer tissue from a Grade 2 patient as denoted in figure. E-H are photographs of the corresponding serial sections that were stained with the secondary antibody alone. FIG. 59 shows photographs of colon cancer tissues stained with humanized MN-E6-scFv-Fc anti-MUC1* antibody at 50 ug/mL, then stained with a secondary goat-anti-human HRP antibody. A-D are colon cancer tissue from a metastatic patient as denoted in figure. E-H are photographs of the corresponding serial sections that were stained with the secondary antibody alone. FIG. 60 shows photographs of prostate cancer tissues stained with humanized MN-E6-scFv-Fc anti-MUC1* antibody at 50 ug/mL, then stained with a secondary goat-anti-human HRP antibody. A-D are prostate cancer tissue from a patient as denoted in figure. E-H are photographs of the corresponding serial sections that were stained with the secondary antibody alone. FIG. 61 shows photographs of prostate cancer tissues stained with humanized MN-E6-scFv-Fc anti-MUC1* antibody at 50 ug/mL, then stained with a secondary goat-anti-human HRP antibody. A-D are prostate cancer tissue from a patient as denoted in figure. E-H are photographs of the corresponding serial sections that were stained with the secondary antibody alone. FIG. 62 shows photographs of prostate cancer tissues stained with humanized MN-E6-scFv-Fc anti-MUC1* antibody at 50 ug/mL, then stained with a secondary goat-anti-human HRP antibody. A-D are prostate cancer tissue from a patient as denoted in figure. E-H are photographs of the corresponding serial sections that were stained with the secondary antibody alone.

One aspect of the invention is a method for treating a patient diagnosed with, suspected of having, or at risk of developing a MUC1 positive or MUC1* positive cancer, wherein a specimen is obtained from the patient's cancer and is tested for reactivity with an antibody that binds to PSMGFR SEQ ID NO:2, SNIKFRPGSVVVQLTLAFREGTINVHDVETQFNQYKTEAASRY (SEQ ID NO:620) or SVVVQLTLAFREGTINVHDVETQFNQYKTEAASRY (SEQ ID NO:621). The patient is then treated with an scFv, scFv-Fc or CAR T that comprises antibody variable fragments from the antibody that reacted with their cancer specimen. Another aspect of the invention is a method for treating a patient diagnosed with, suspected of having, or at risk of developing a MUC1 positive or MUC1* positive cancer, wherein a specimen is obtained from the patient's cancer and is tested for reactivity with MN-E6-scFv, MN-C2-scFv, MN-C3-scFv or MN-C8-scFv; the patient is then treated with the scFv, scFv-Fc-mut or CAR T that comprises portions of the antibody that reacted with their cancer specimen.

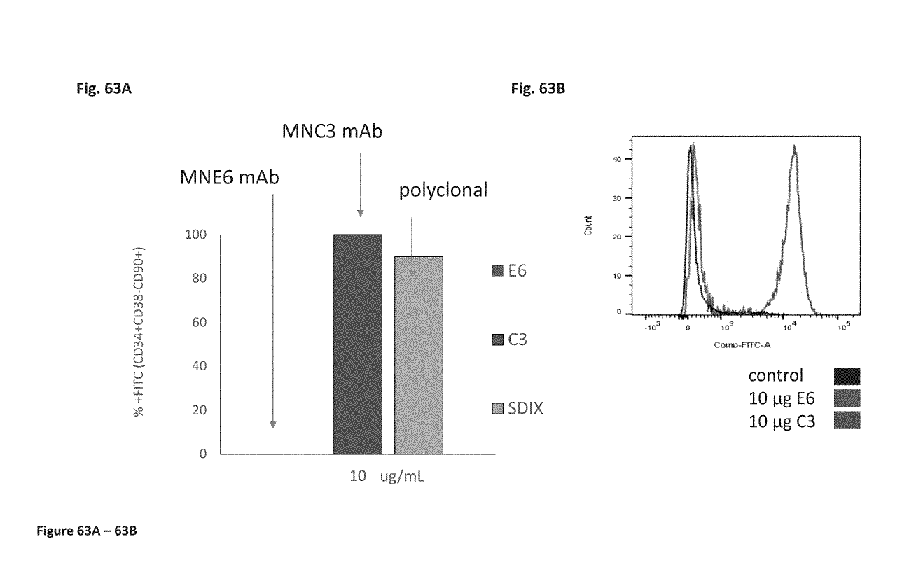

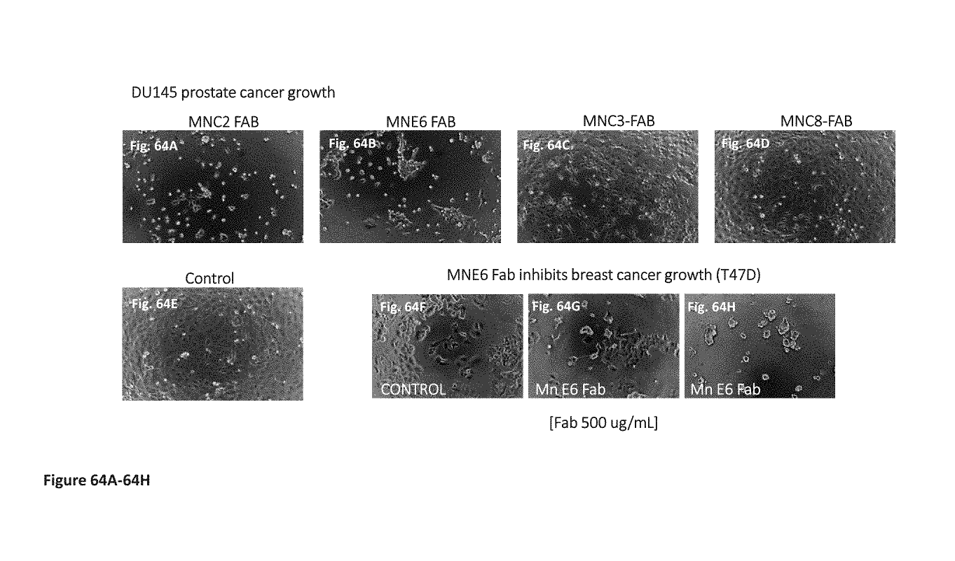

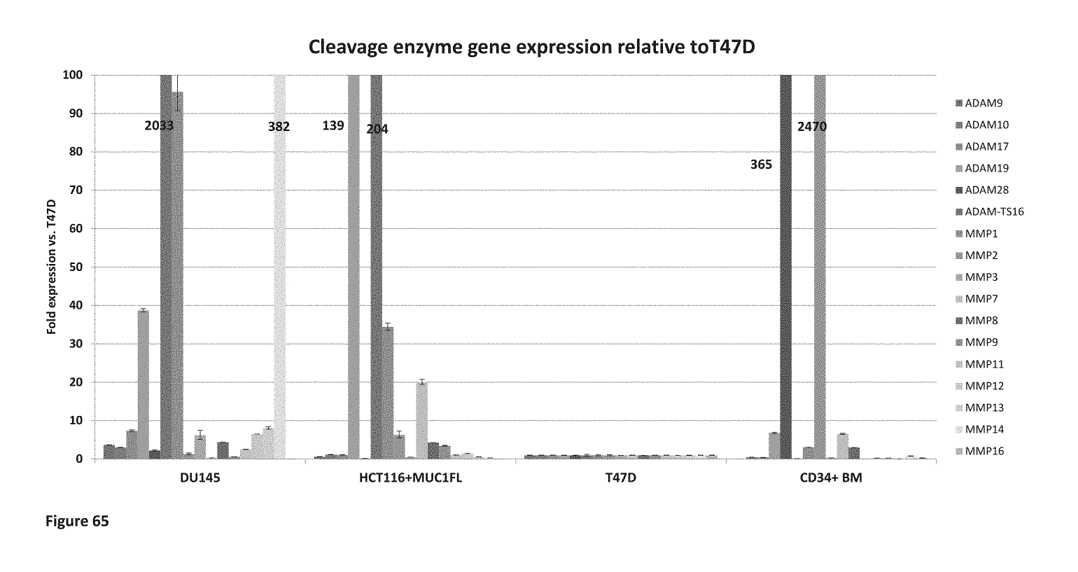

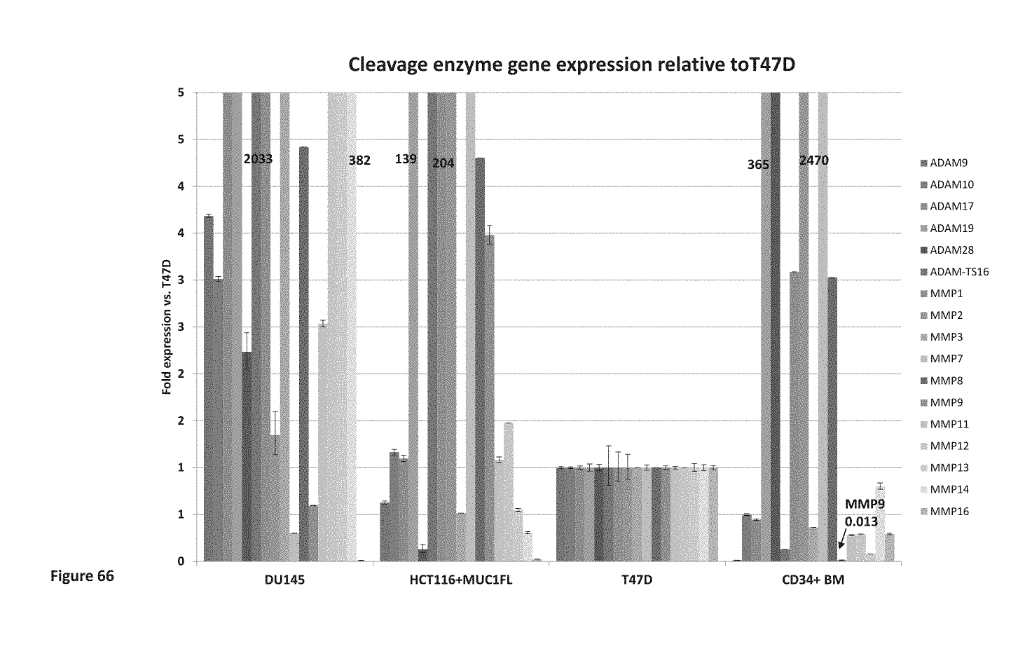

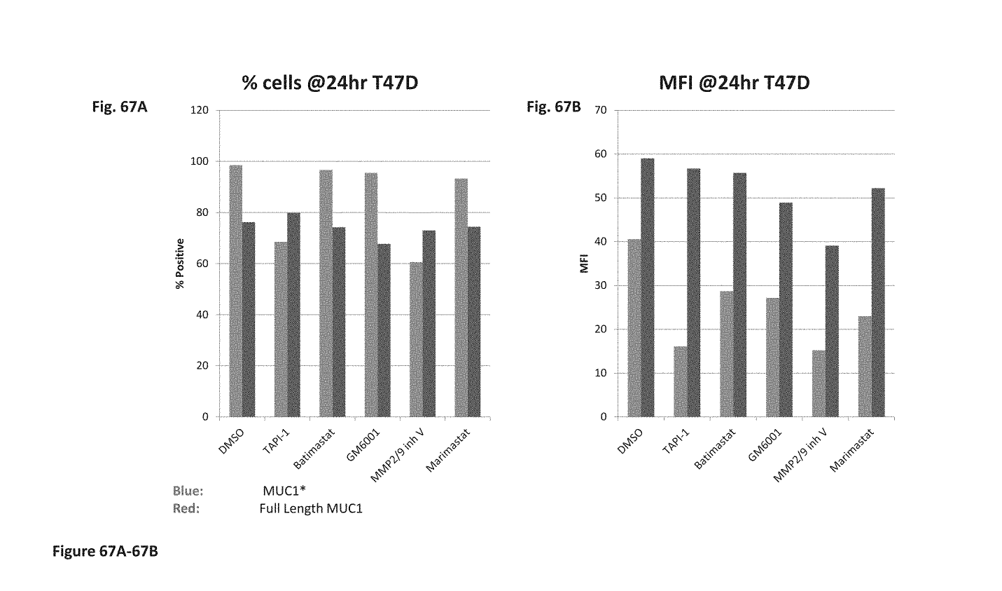

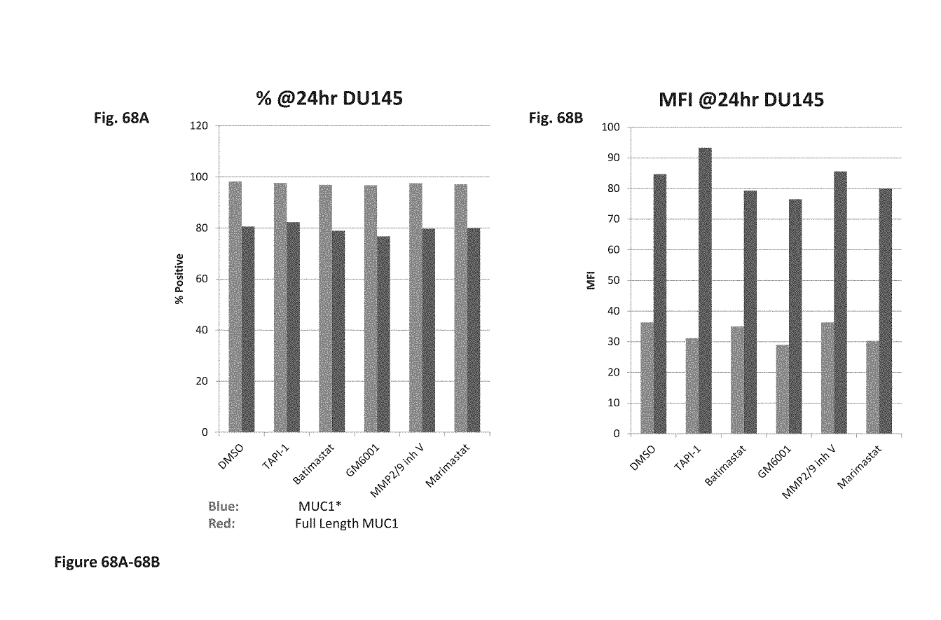

We discovered that MUC1 can be cleaved to MUC1* by more than one cleavage enzyme and that the site of cleavage affects its fold and consequently affects which monoclonal antibody is able to recognize that form of MUC1*. Different cancer cells or cancerous tissues express different cleavage enzymes. We tested various cleavage enzyme inhibitors on different cancer cell lines and found that an inhibitor that inhibits cleavage of MUC1 in one cancer cell line did not inhibit its cleavage in another cancer cell line. Similarly, PCR experiments showed that cleavage enzymes are expressed at different levels in different cells or cell lines. For example, hematopoietic stem cells of the bone marrow express a MUC1* that is recognized by monoclonal antibody MNC3 but not MNE6 or MNC2 (FIG. 63). The growth of DU145 prostate cancer cells and T47D breast cancer cells is inhibited by the Fabs of MNC2 and MNE6 but not by the Fabs of MNC3 or MNC8, indicating that the cancer cell lines express a MUC1* that is recognized by MNE6 and MNC2 but not by MNC3 or MNC8 (FIG. 64). PCR experiments show that CD34 positive cells of the bone marrow express about 2,500-times more MMP2 and about 350-times more ADAM28 than T47D breast cancer cells, while DU145 prostate cancer cells express about 2,000-times more ADAM TS16, about 400-times more MMP14 and about 100-times more MMP1 than T47D breast cancer cells (FIG. 65 and FIG. 66). Conversely, T47D breast cancer cells express about 80-times more MMP9 than the bone marrow cells and about twice as much as DU145 prostate cancer cells. Various cleavage enzyme inhibitors were tested for their ability to inhibit cleavage in different kinds of cancer cells. TAPI-1 that inhibits MMP2, MMP9, and ADAM17 and MMP2/9 V inhibitor that inhibits MMP2, MMP9, MMP14, inhibited the cleavage of MUC1 in T47D breast cancer cells (FIGS. 67A, 67B), but none of the cleavage enzyme inhibitors tested had an effect in DU145 prostate cancer cells (FIGS. 68A, 68B). These experiments indicated that MUC1 in these breast cancer cells was being cleaved by MMP2, MMP9, MMP14 or ADAM17 or combinations of these enzymes.

BiTEs

Divalent (or bivalent) single-chain variable fragments (di-scFvs, bi-scFvs) can be engineered by linking two scFvs. This can be done by producing a single peptide chain with two V_{H} and two V_{L} regions, yielding tandem scFvs. Another possibility is the creation of scFvs with linker peptides that are too short for the two variable regions to fold together (about five amino acids), forcing scFvs to dimerize. This type is known as diabodies. Diabodies have been shown to have dissociation constants up to 40-fold lower than corresponding scFvs, meaning that they have a much higher affinity to their target. Consequently, diabody drugs could be dosed much lower than other therapeutic antibodies and are capable of highly specific targeting of tumors in vivo. Still shorter linkers (one or two amino acids) lead to the formation of trimers, so-called triabodies or tribodies. Tetrabodies have also been produced. They exhibit an even higher affinity to their targets than diabodies.

All of these formats can be composed from variable fragments with specificity for two different antigens, in which case they are types of bispecific antibodies. The furthest developed of these are bispecific tandem di-scFvs, known as bi-specific T-cell engagers (BiTE antibody constructs). BiTEs are fusion proteins consisting of two scFvs of different antibodies, on a single peptide chain of about 55 kilodaltons. One of the scFvs may bind to T cells such as via the CD3 receptor, and the other to a tumor cell via a tumor specific molecule, such aberrantly expressed MUC1*.

Another aspect of the invention is a method for treating a patient diagnosed with, suspected of having, or at risk of developing a MUC1 positive or MUC1* positive cancer, wherein the patient is administered an effective amount of a BiTE wherein one antibody variable fragment of the BiTE binds to a T cell surface antigen and the other antibody variable fragment of the BiTE binds to PSMGFR SEQ ID NO:2, SNIKFRPGSVVVQLTLAFREGTINVHDVETQFNQYKTEAASRY (SEQ ID NO:620) or SVVVQLTLAFREGTINVHDVETQFNQYKTEAASRY (SEQ ID NO:621). In one case, the antibody variable fragment of the BiTE that binds to MUC1* comprises portions of huMN-E6, huMN-C2, huMN-C3, or huMN-C8.

In another aspect of the invention, MUC1* peptides including PSMGFR SEQ ID NO:2, most or all of SNIKFRPGSVVVQLTLAFREGTINVHDVETQFNQYKTEAASRY (SEQ ID NO:620) or SVVVQLTLAFREGTINVHDVETQFNQYKTEAASRY (SEQ ID NO:621) are used in adoptive T cell approaches. In this case, a patient's T cells are exposed to the MUC1* peptides and through various rounds of maturation, the T cells develop MUC1* specific receptors. The adapted T cells are then expanded and administered to the donor patient who is diagnosed with, suspected of having, or is at risk of developing a MUC1* positive cancer.

A series of CARs were also made that had MNC2 and humanized MNC2 as the extra cellular, targeting head of the CAR. The constructs for these CARs were inserted into a plasmid that was then inserted into a Lenti viral vector. Human T cells were then transduced with the lenti viral vector carrying the MNC2 CARs and huMNC2 CARs. MNC2-scFv-CARs that were mouse sequence or humanized were generated. CARs comprising MNC2-scFv and a variety of transmembrane and intracellular co-stimulatory domains were generated including constructs listed in Table 1. In one aspect of the invention, the CAR comprised huMNC2-scFv-short hinge region-transmembrane domain derived from CD8-short intracellular piece-4-1BB-3zeta. In another aspect, the transmembrane domain was derived from CD4 transmembrane sequence. In another aspect, the intracellular co-stimulatory domain was CD28-3zeta. In yet another aspect, the intracellular co-stimulatory domain was CD28-4-1BB-3zeta.

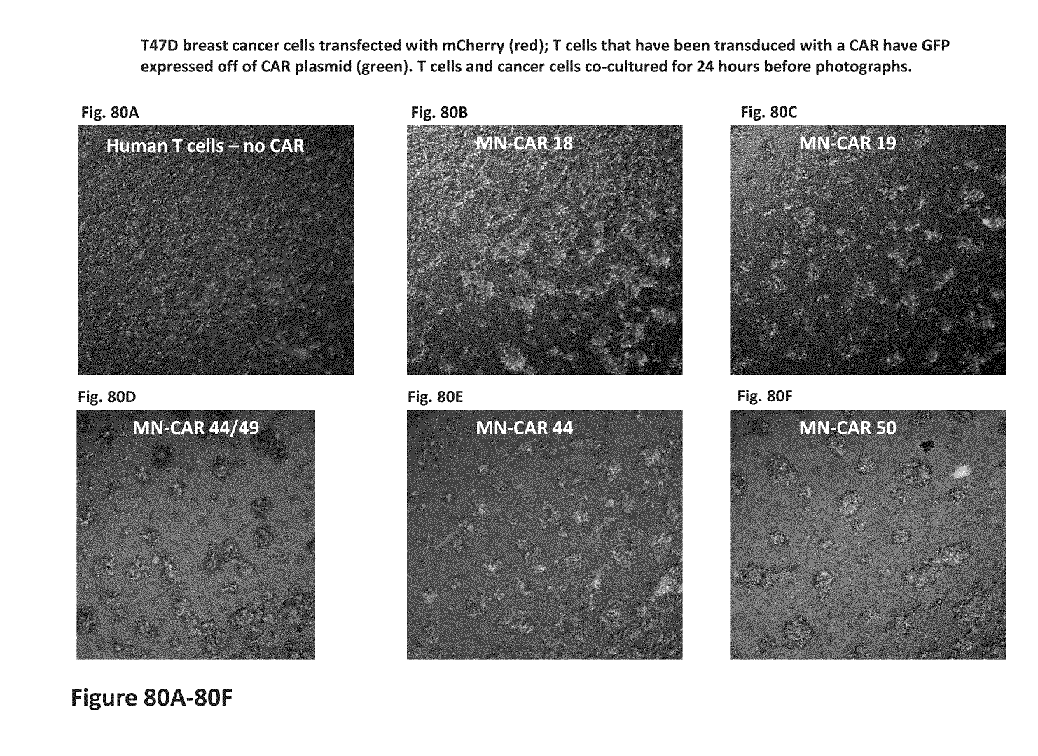

There are a variety of methods for assessing whether or not T cells recognize a target cell and are in the process of mounting an immune response. T cells cluster when they recognize a target or foreign cell. This can be readily seen with the naked eye or at low magnification. The appearance of CAR T cell clustering when co-cultured with target cancer cells is one measure of: a) whether or not they recognize the cells as target cells; and b) whether or not they are getting activated to attack the targeted cells, which in this case are cancer cells. FIGS. 80A-80F show photographs of MUC1* positive T47D breast cancer cells that were stably transfected with mCherry, so are red, which were co-cultured with either human T cells without a CAR or human T cells transduced with huMNC2-scFv-CAR44, huMNC2-scFv-CAR49, huMNC2-scFv-CAR50, huMNC2-scFv-CAR18 or huMNC2-scFv-CAR19. In this case, the CAR constructs carry a GFP marker so the CAR transduced T cells are green. As can be seen, there is no T cell induced clustering of the cancer cells when the T cell does not carry a CAR. However, when T cells carrying a MUC1* targeting CAR, there is dramatic clustering of the MUC1* positive cancer cells.

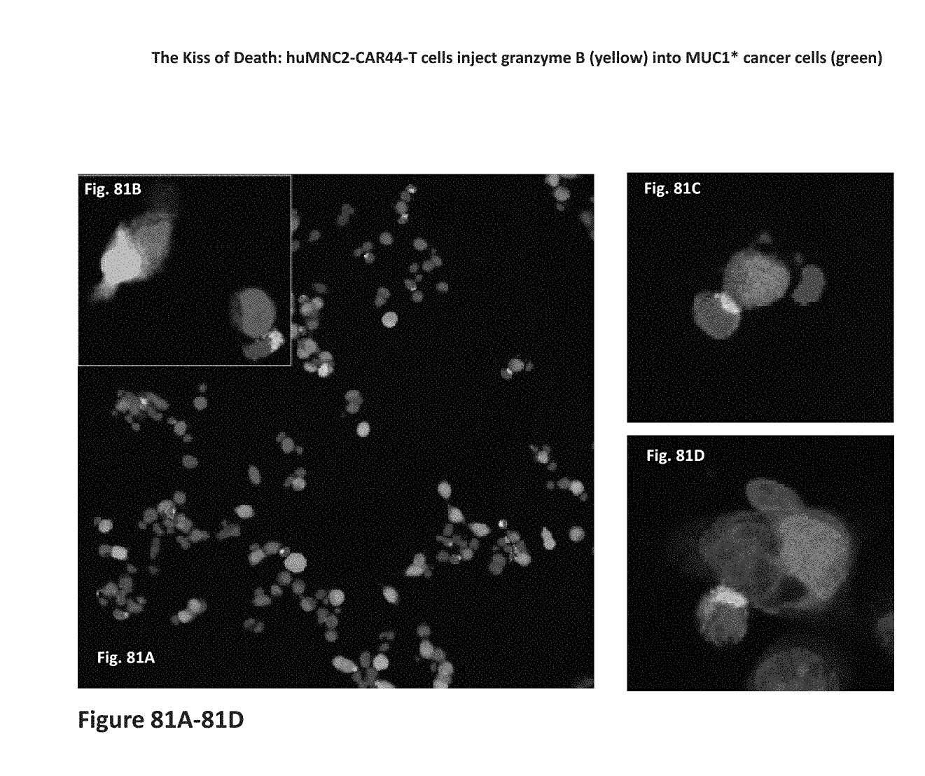

After T cells recognize and cluster target cells, they overexpress perforin and granzyme B. Together these two molecules activate a cell death pathway in the targeted cell. It is thought that the perforin makes a hole in the target cell into which the T cell injects granzyme B which then activates apoptotic proteases, causing the target cell to lyse. FIGS. 81A-81D show huMNC2-scFV-CAR44 T cells binding to target MUC1* positive prostate cancer cells and injecting granzyme B.

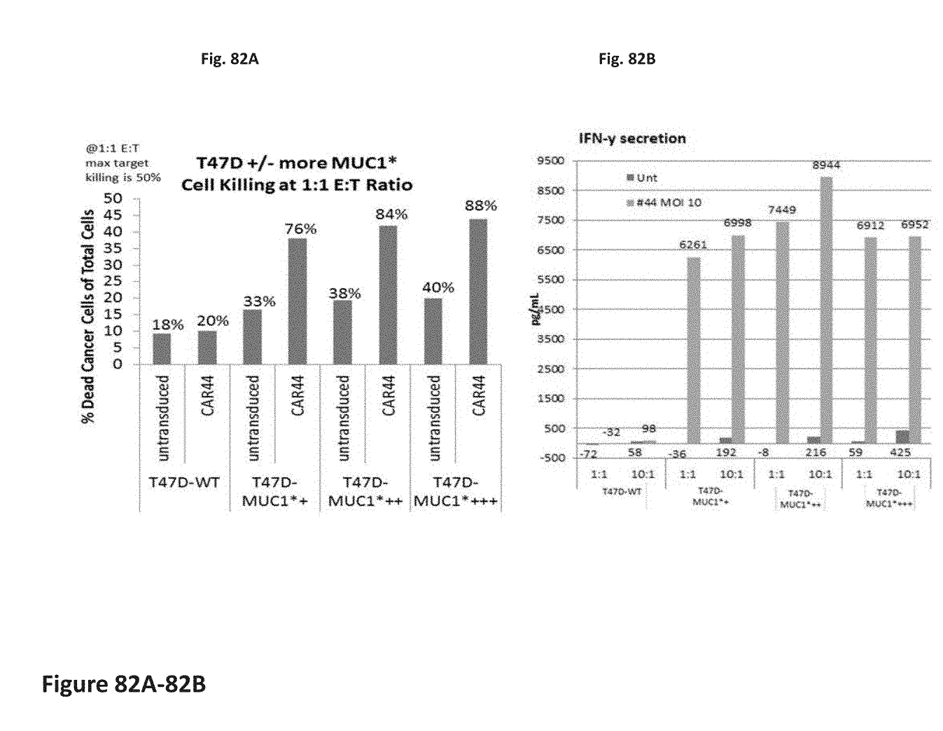

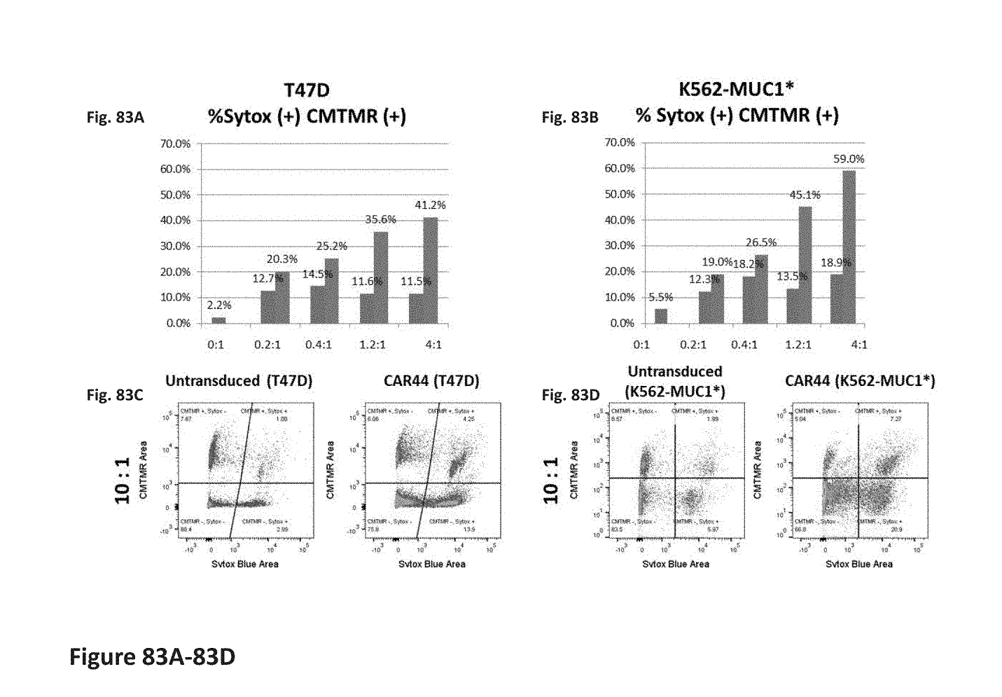

Another measure of whether or not a T cell has recognized a target cell and is activated to kill that cell, is the upregulation of cytokines, especially interferon gamma (IFN-g). Table 2 lists the results of ELISA experiments measuring the amount of interferon gamma secreted by a variety of MUC1* targeting CAR T cells after co-culture with a variety of different cancer cells. To establish the link between MUC1* expression and CAR T activity, we performed an experiment to determine if the amount of CAR T killing was proportional to the amount of MUC1* expressed by the cancer cell. T47D is a highly MUC1* positive breast cancer cell. These cells also express some full-length MUC1. T47D cells were transfected with varying amounts of additional MUC1* then co-cultured with CAR T cells. The results showed that at low effector (CAR T) to target (cancer cells) ratios such as 1:1, specific CAR T killing increased with increasing MUC1* expression and the amount of secreted interferon gamma also increased with increasing MUC1* (FIG. 82B). Another method for measuring CAR T response is by fluorescence activated cell sorting (FACS). FIG. X7A shows a graph of FACS analysis of the T47D cancer cells transfected with additional MUC1*. At a 1:1 E:T ratio, CAR T mediated cancer cell killing increased as the amount of MUC1* expressed on the cancer cells increased. This is important because we previously showed that as cancer cells acquire resistance to chemotherapy agents, they increase the amount of MUC1* that they express (Fessler et al 2009). Therefore an anti-MUC1* CAR T would be especially beneficial as a treatment for cancer patients with late stage cancers or those that have acquired resistance to chemotherapy agents. Several MNC2-scFv-CARs were transduced into human T cells and analyzed by FACS to determine their ability to kill targeted MUC1* positive cancer cells. FIGS. 83A-83D show the results of FACS analysis of huMNC2-CAR44 T cells after 24 hours of co-culture with MUC1* positive cancer cells. FIG. 83A is a graph of FACS data showing the percentage of T47D cancer cells that were killed by huMNC2-CAR44 T cells (blue bars), compared to untransduced T cells (red bars). The X-axis shows the ratio of T cells to cancer cells. FIG. 83B is a graph of FACS data showing the percentage of K562-MUC1* cancer cells that were killed by huMNC2-CAR44 T cells (blue bars), compared to untransduced T cells (red bars). FIG. 83C shows the FACS scans wherein the T47D breast cancer cells were stained with the dye CMTMR. Sytox blue is a dead cell stain. Dead cancer cells are those in quadrants 2 and 3. FIG. 83D shows the FACS scans wherein the K562-MUC1* cancer cells were stained with the dye CMTMR. Sytox blue is a dead cell stain. Dead cancer cells are those in quadrants 2 and 3.

In addition to FACS analysis, many researchers now use an xCELLigence instrument to measure CAR T killing of cancer cells. FACS is not the best method for tracking T cell induced cell killing because the T cells lyse the target cell. By FACS it is difficult to measure dead cells because they are excluded as cell debris, so one must infer an amount of cell killing and by various methods determine if the missing cells are T cells or cancer cells.

The xCELLigence instrument uses electrode arrays upon which cancer cells are plated. The adherent cancer cells insulate the electrode and so cause an increase in impedance as they grow. Conversely, T cells are not adherent and remain in suspension so do not contribute to insulation of the electrode which would increase impedance. However, if the T cells or CAR T cells kill the cancer cells on the electrode plate, the cancer cells ball up and float off as they die, which causes the impedance to decrease. The xCELLigence instrument measures impedance as a function of time, which is correlated to cancer cell killing. In addition, the electrode plates also have a viewing window. When CAR T cells effectively kill the adsorbed target cancer cells, there is a decrease in impedance but also one can see that there are no cancer cells left on the plate surface.

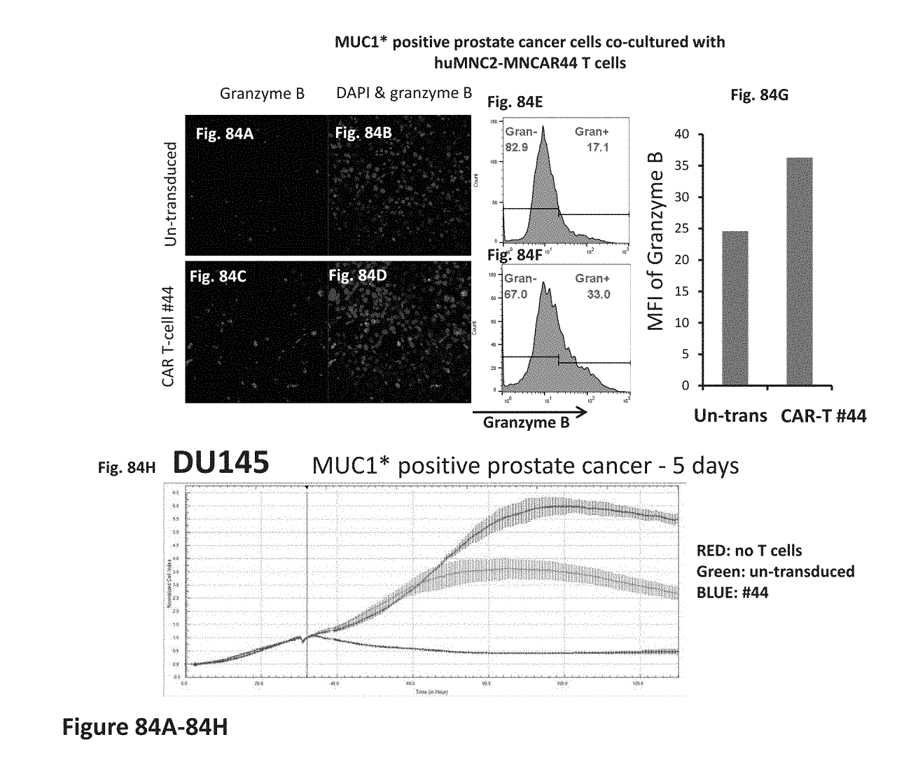

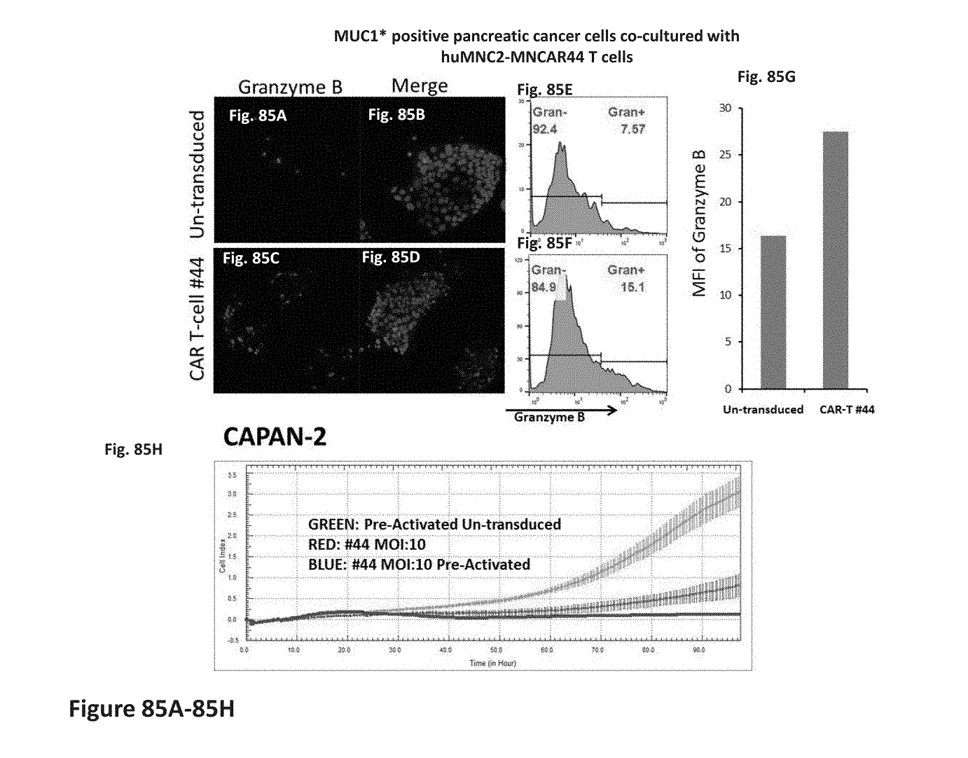

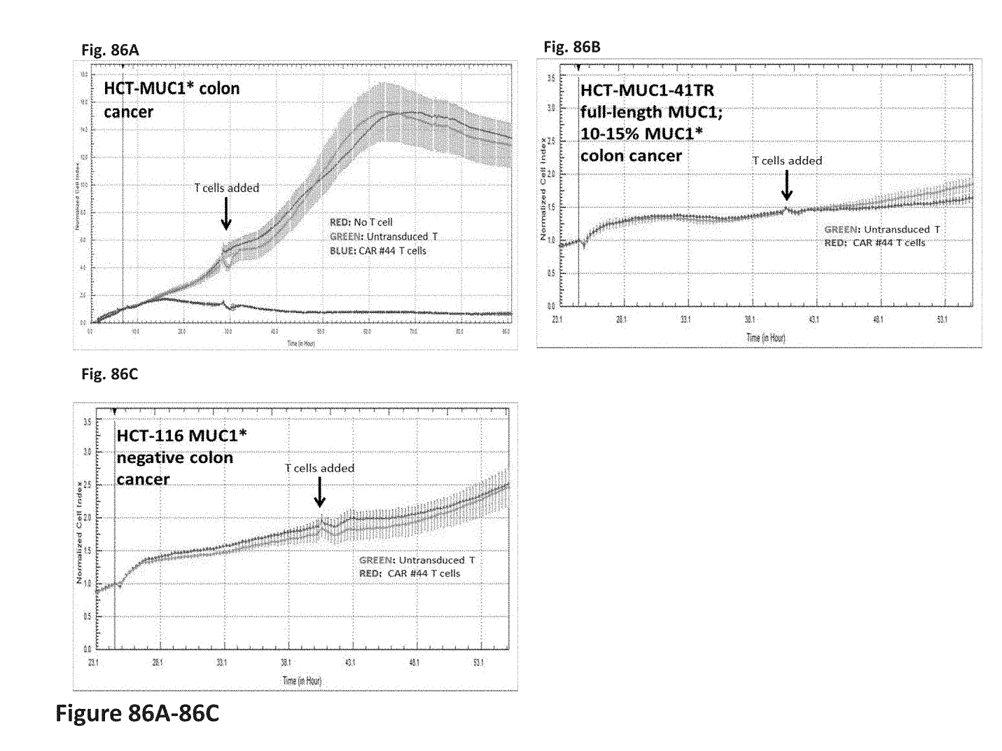

FIGS. 84A-84H show the cytotoxic effect of huMNC2-CAR44 T cells on MUC1* positive DU145 prostate cancer cells as measured by a variety of assays. FIG. 84A is a fluorescent photograph of untransduced T cells co-cultured with the prostate cancer cells, wherein granzyme B is stained with a red fluorophore. FIG. X4B is the DAPI and granzyme B merge. FIG. 84C is a fluorescent photograph of huMNC2-CAR44 T cells co-cultured with the prostate cancer cells, wherein granzyme B is stained with a red fluorophore. FIG. 84D is the DAPI and granzyme B merge. FIG. 84E is a FACS scan for fluorescently labeled granzyme B for untransduced T cells incubated with the cancer cells. FIG. 84F is a FACS scan showing a positive increase in fluorescently labeled granzyme B for huMNC2-CAR44 T cells incubated with the cancer cells. FIG. 84G is a graph of the mean fluorescent intensity. FIG. 84H is an xCELLigence scan tracking the real-time killing of DU145 cancer cells by huMNC2-CAR44 T cells (blue trace) but not by untransduced T cells (green). FIGS. 85A-85H show the cytotoxic effect of huMNC2-CAR44 T cells on MUC1* positive CAPAN-2 pancreatic cancer cells as measured by a variety of assays. FIG. 85A is a fluorescent photograph of untransduced T cells co-cultured with the pancreatic cancer cells, wherein granzyme B is stained with a red fluorophore. FIG. 85B is the DAPI and granzyme B merge. FIG. 85C is a fluorescent photograph of huMNC2-CAR44 T cells co-cultured with the pancreatic cancer cells, wherein granzyme B is stained with a red fluorophore. FIG. 85D is the DAPI and granzyme B merge. FIG. 85E is a FACS scan for fluorescently labeled granzyme B for untransduced T cells incubated with the cancer cells. FIG. 85F is a FACS scan showing a positive increase in fluorescently labeled granzyme B for huMNC2-CAR44 T cells incubated with the cancer cells. FIG. 85G is a graph of the mean fluorescent intensity. FIG. 85H is an xCELLigence scan tracking the real-time killing of CAPAN-2 cancer cells by huMNC2-CAR44 T cells (blue trace) but not by untransduced T cells (green). FIGS. 86A-86C show xCELLigence scans tracking the real-time killing of MUC1* positive cancer cells, but not MUC1* negative cells, by huMNC2-CAR44 T cells. FIG. 86A shows that huMNC2-CAR44 T cells effectively kill HCT colon cancer cells that have been stably transfected with MUC1*. FIG. 86B shows that huMNC2-CAR44 T cells have almost no effect on HCT-MUC1-41TR, which is a MUC1 negative cancer cell that has been stably transfected with a MUC1 full-length. In this cell line only about 10% of the cell have MUC1 cleaved to MUC1*. FIG. 86C shows that huMNC2-CAR44 T cells have no effect on HCT-116 cells, which is a MUC1 negative colon cancer cell line.

These data demonstrate that T cells transduced with a CAR wherein the antibody fragment targeting head is MNC2, effectively kill MUC1* positive cancer cells. These data specifically show that huMNC2-scFV-CAR44 transduced into human T cells effectively kill MUC1* positive cancer cells. Because we and others have now demonstrated that the most important aspect of CAR T function is the targeting antibody fragment, it follows that an immune cell or a T cell transduced with any CAR having the antibody fragment MNC2-scFV or huMNC2-scFV would have similar efficacy against MUC1 or MUC1* positive tumors. For example, the hinge region that connects the scFv to the transmembrane portion could be any flexible linker. The intracellular co-stimulatory domains could be CD28-3zeta, CD28-4-1BB-3zeta or any combination of immune cell co-stimulatory domains.

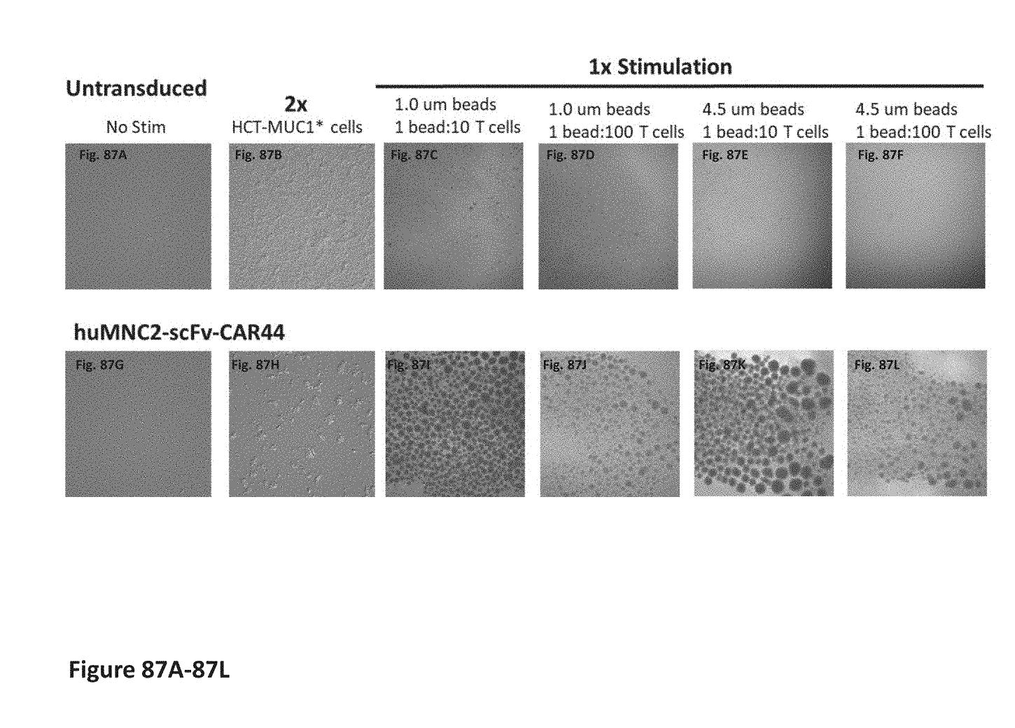

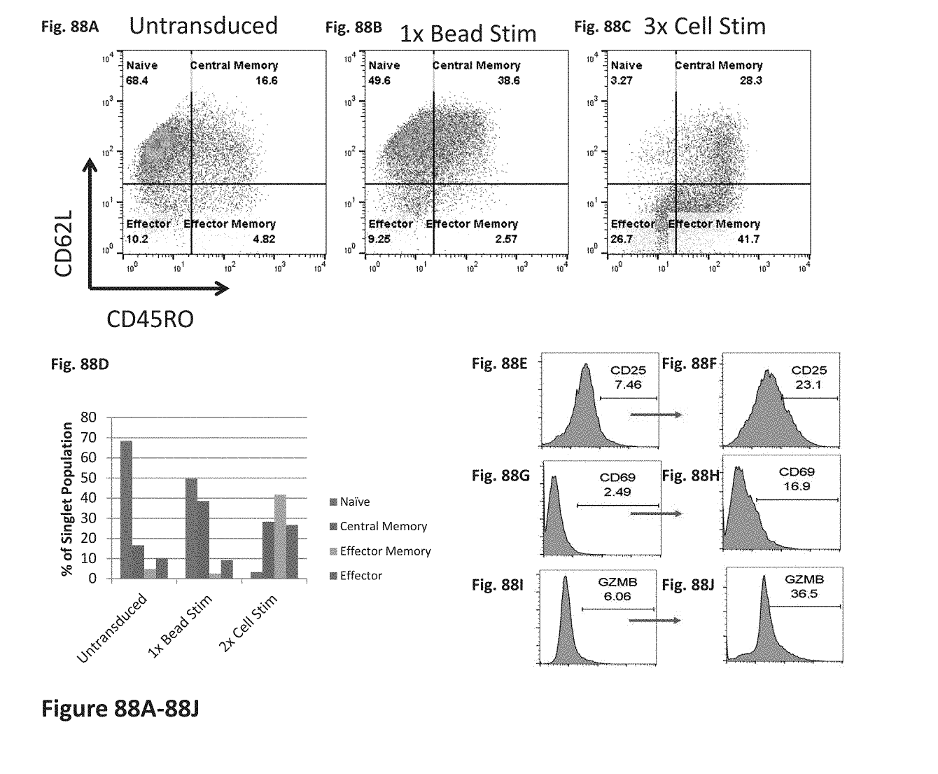

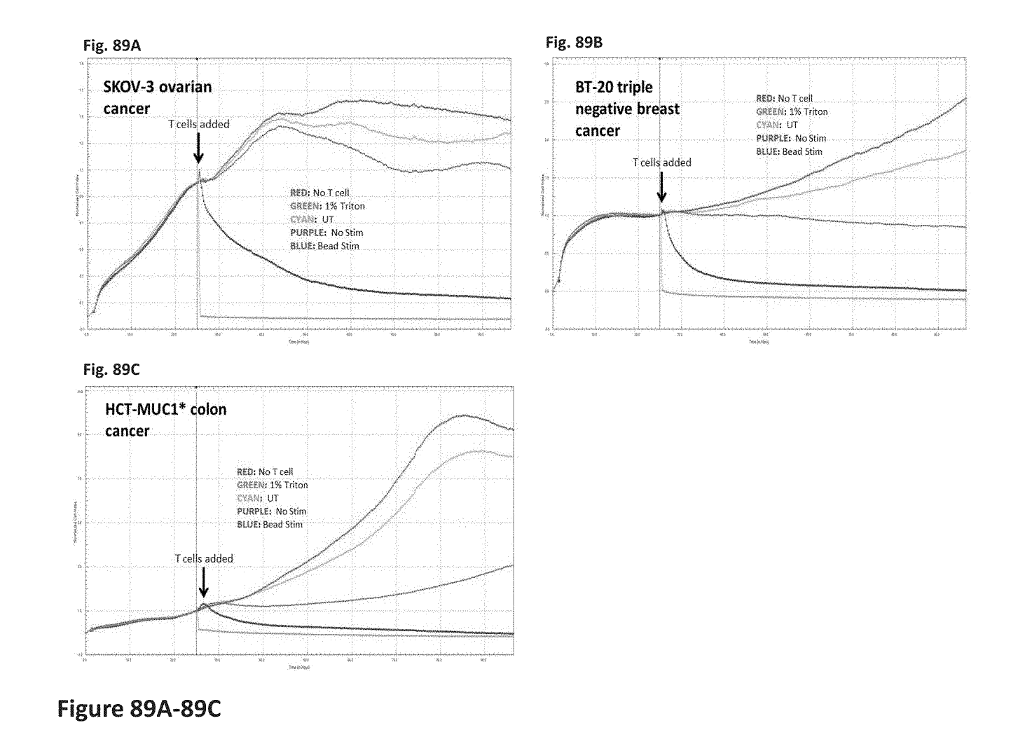

Experiments were also performed exploring methods of pre-activating the CAR T cells to more effectively kill the target cancer cells. We first tested pre-stimulation of the CAR T cells using beads presenting anti-CD3 and anti-CD28 antibodies. This pre-stimulation increased the amount of cell killing but the increase was not specific for the target of the CAR. Rather, the CD3-CD28 stimulated CAR T cells non-specifically killed MUC1* positive and negative cells. We next tried pre-stimulating the CAR T cells with either beads or cancer cells that expressed the target of the antibody portion of the CAR. A synthetic MUC1*_{extra cellular domain} peptide was attached to either 1 μm or 4.5 μm beads. Anti-MUC1* CAR T cells were incubated with the peptide presenting beads for 12-24 hours. FIGS. 87A-87L show the untransduced T cells or the CAR T cells after 24 hour incubation with MUC1* peptide presenting beads. As can be seen, only the CAR transduced T cells show activation-induced clustering. The CAR T cells were separated from the beads by centrifugation, then analyzed by FACS to measure expression of T cell activation markers CD25, CD69 and granzyme B. As can be seen in FIGS. 88A-88D, T cell activation markers increase after incubation with MUC1* presenting beads if and only if the T cell had been transduced with a CAR whose extra cellular domain comprised an anti-MUC1* antibody fragment. In sharp contrast to pre-activation with CD3-CD28 beads, stimulation with MUC1* peptide beads only increased specific killing. There was no increase in the killing of MUC1* negative cells. FIGS. 89A-89C show xCELLigence scans that show the enhanced killing of bead-stimulated anti-MUC1* CAR T cells on human ovarian cancer cells, triple negative breast cancer cells and a MUC1 negative colon cancer cell line that was stably transfected with MUC1*. The enhanced killing capability of MUC1* peptide bead stimulated CAR T cells enabled the CAR T cells to effectively kill target cancer cells for longer periods of time and at much lower T cell to cancer cell ratios. In one aspect of the invention, CAR T cells are pre-stimulated by incubation with beads or surfaces that present a peptide derived from the MUC1* extra cellular domain, before administering to a patient diagnosed with or at risk of developing a MUC1* positive cancer.

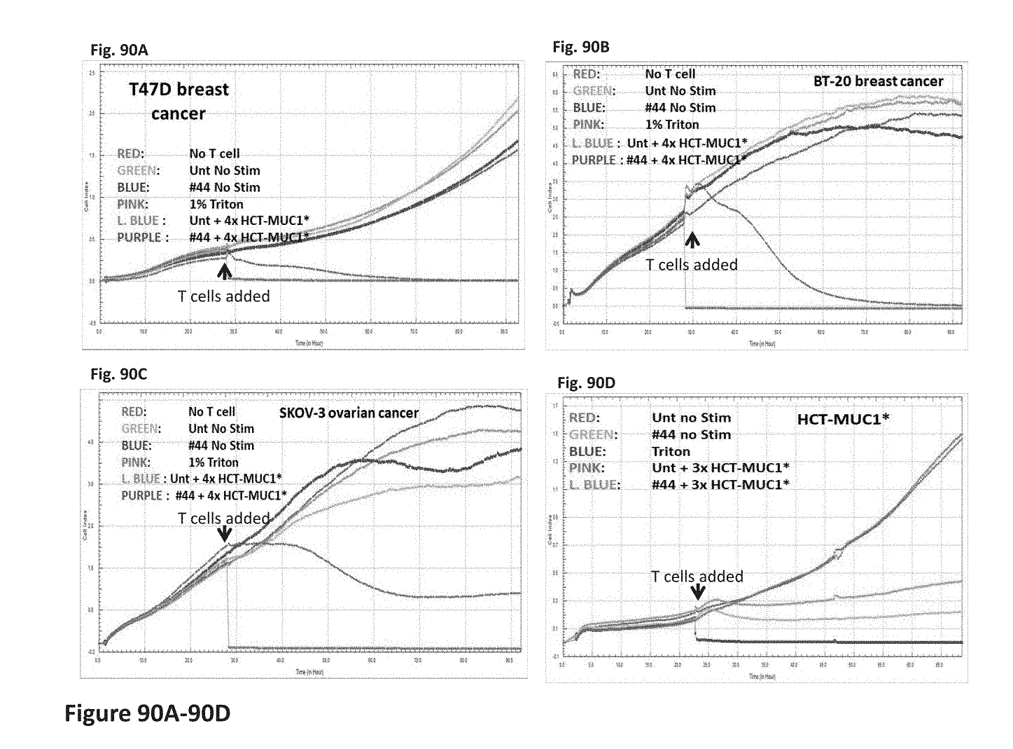

We also tested pre-activating CAR T cells by incubating them with cancer cells that present the target antigen. We incubated huMNC2-CAR44 T cells with HCT-MUC1* cells for 12-24 hours. This pre-stimulation was done once, twice, three or four times. Target cell pre-stimulation also greatly enhanced the specific killing of CAR T cells. As can be seen in FIGS. 90A-90D, specific cell killing by the cancer cell stimulated CAR T cells increased their killing potential even at low CAR T to cancer cell ratios and for longer periods of time. FIGS. 90A-90D show cancer cell stimulated huMNC2-scFv-CAR44 transduced human T cells effectively kill T47D breast cancer cells, BT-20 triple negative breast cancer cells, SKOV-3 ovarian cancer cells and HCT-MUC1* cancer cells. In one aspect of the invention, CAR T cells are pre-stimulated by incubation with MUC1* expressing cells, which may be cancer cells, before administering to a patient diagnosed with or at risk of developing a MUC1* positive cancer. In a preferred embodiment, the MUC1* stimulation cells are UV or chemically inactivated before co-culture with the CAR T cells.

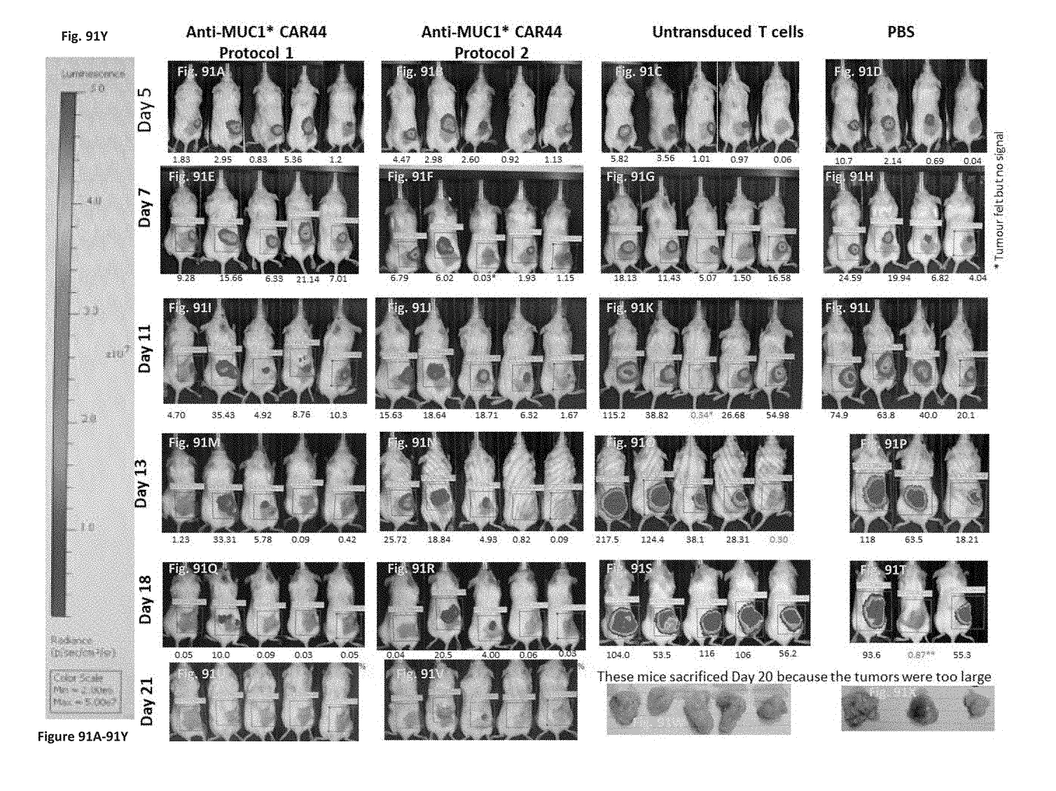

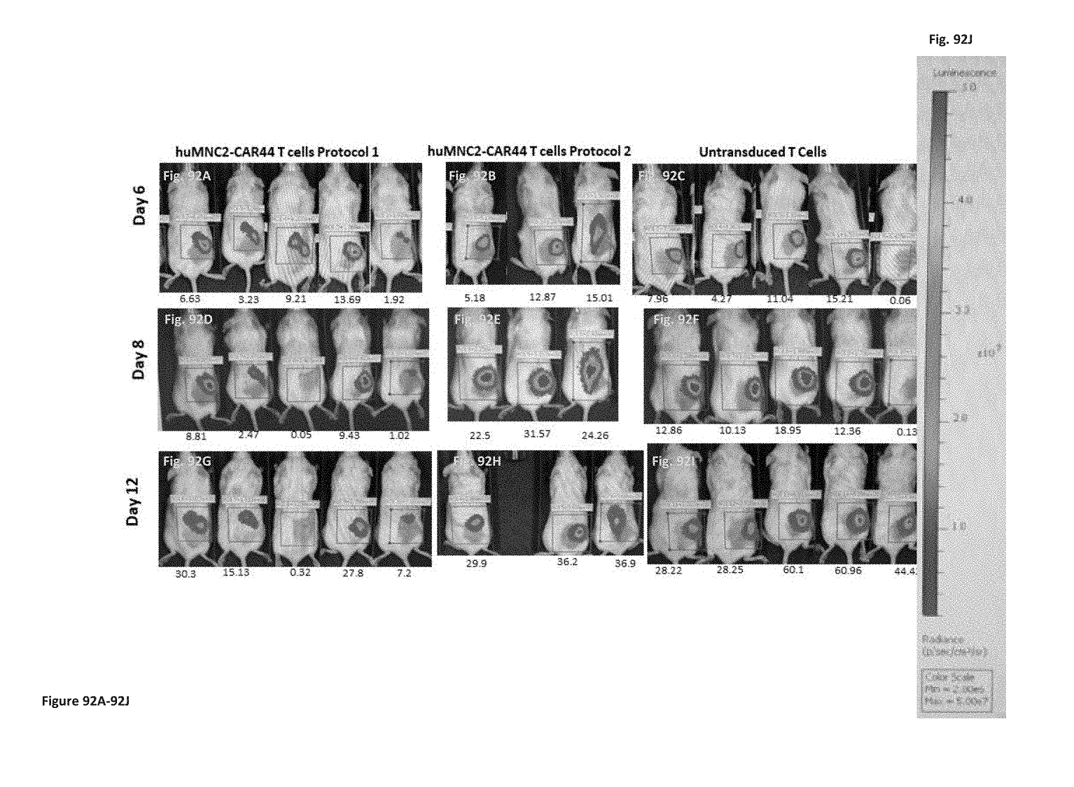

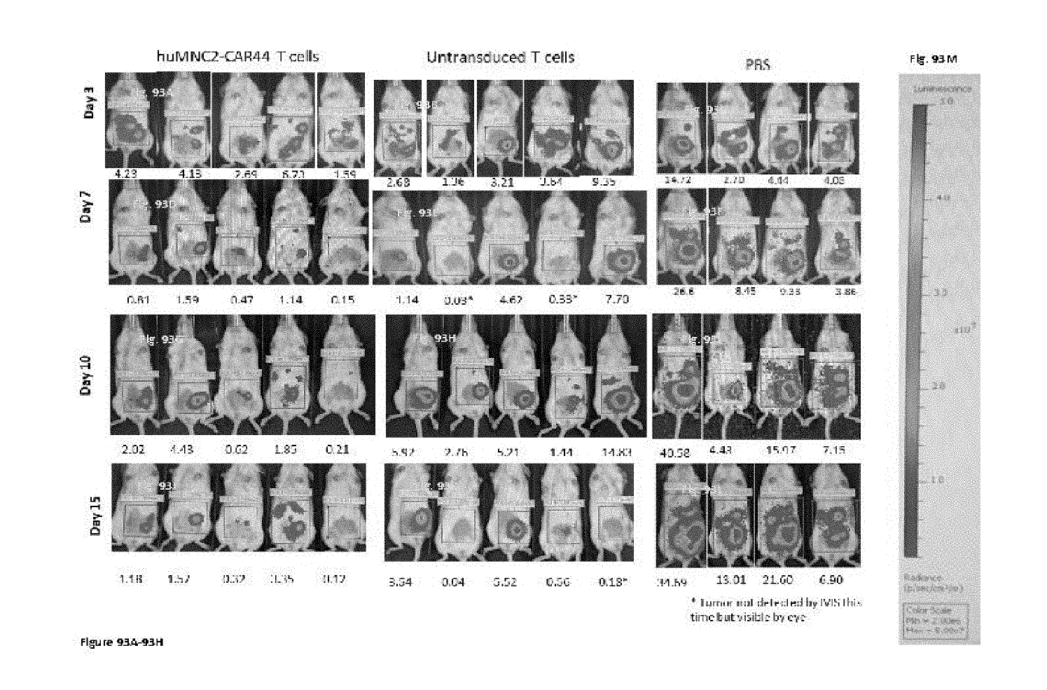

huMNC2-scFv-CAR44 transduced human T cell that were bead stimulated (Protocol 1) or cancer cell stimulated (Protocol 2) were tested for their ability to inhibit tumor growth in animals. Human cancer cells that had been stably transfected with Luciferase were injected into female NOD/SCID/GAMMA (NSG) mice between 11 and 15 weeks of age. In one experiment, 500,000 HCT-MUC1* cancer cells were injected sub-cutaneously into a rear flank. Tumor engraftment was verified by injecting the animals with Luciferin and then imaging the fluorescent cancer cells using an IVIS instrument. IVIS images taken Day 5 post implantation showed the presence of tumor cells. On Day 6 and on Day 12, 10M huMNC2-scFv-CAR44 T cells were administered to the animals. 5M of the CAR T cells were administered by intratumor injection and the other 5M were administered by tail vein injection. Control groups were injected by same administration routes with either the same number of untransduced T cells or same volume of PBS. IVIS measurements of tumor burden were taken on Days 7, 11, 13, and 21. As can be seen in FIGS. 91A-91Y, both groups of control mice had tumors that continuously grew, whereas the mice treated with bead-stimulated huMNC2-scFv-CAR44 T cells have no detectable cancer cells by Day 21. Three (3) of the five (5) mice treated with cancer cell-stimulated huMNC2-scFv-CAR44 T cells have no detectable cancer cells by Day 21. The other two (2) mice have a barely detectable number of cancer cells remaining by Day 21.

huMNC2-scFv-CAR44 transduced human T cell that were bead stimulated (Protocol 1) or cancer cell stimulated (Protocol 2) were also tested for their ability to inhibit tumor growth in animals. Human cancer cells that had been stably transfected with Luciferase were injected into female NOD/SCID/GAMMA (NSG) mice between 11 and 15 weeks of age. In another experiment, 500,000 BT-20 MUC1* positive triple negative breast cancer cells were injected sub-cutaneously into a rear flank. Tumor engraftment was verified by injecting the animals with Luciferin and then imaging the fluorescent cancer cells using an IVIS instrument. IVIS images taken Day 6 post implantation showed the presence of tumor cells. On Day 6, after IVIS imaging, 10M huMNC2-scFv-CAR44 T cells were administered to the animals. 5M of the CAR T cells were administered by intratumor injection and the other 5M were administered by tail vein injection. Control group was injected by same administration routes with the same number of untransduced T cells. IVIS measurements of tumor burden were taken on Days 6, 8, and 12. As can be seen in FIGS. 92A-92J, both groups of mice treated with huMNC2-CAR44 T cells showed a decrease in tumor burden compared to the control group.

huMNC2-scFv-CAR44 transduced human T cell that were bead stimulated (Protocol 1) were also tested for their ability to inhibit ovarian cancer growth in animals. Human SKOV-3 MUC1* positive ovarian cancer cells that had been stably transfected with Luciferase were injected into female NOD/SCID/GAMMA (NSG) mice between 11 and 15 weeks of age. In one experiment, 500,000 SKOV-3 cancer cells were injected into the intraperitoneal cavity to mimic metastatic ovarian cancer in humans. Tumor engraftment was verified by injecting the animals with Luciferin and then imaging the fluorescent cancer cells using an IVIS instrument. IVIS images taken Day 3 post implantation showed the presence of tumor cells. On Day 4 and Day 11, post tumor implantation, 10M huMNC2-scFv-CAR44 T cells were IP administered to the animals. On Day 4, CAR T cells were IP injected. On Day 11 half the CAR T cells were injected into the intraperitoneal space and the other half was injected into the tail vein. Control groups were injected by same administration routes with either the same number of untransduced T cells or same volume of PBS. Subsequent IVIS measurements of tumor burden were taken on Day 7, Day 10 and Day 15. As can be seen in FIGS. 93A-93L, control mice have tumors that are growing at a much faster rate than the huMNC2-CAR44 T cell treated mice. FIG. 93M shows the IVIS color bar correlating photons/second to color.

One aspect of the invention is a method for treating a patient diagnosed with, suspected of having, or at risk of developing a MUC1 positive or MUC1* positive cancer, wherein the patient is administered an effective amount of immune cells that have been transduced with a MUC1* targeting CAR, wherein the CAR is chosen from among the group consisting of MN-E6-CD8-3z (SEQ ID NOS:294-295); MN-E6-CD4-3z (SEQ ID NOS:746-747); MN-E6-CD8-CD28-3z (SEQ ID NOS:297-298); MN-E6-CD4-CD28-3z (SEQ ID NOS:748-749); MN-E6-CD8-41BB-3z (SEQ ID NOS:300-301); MN-E6-CD4-41BB-3z (SEQ ID NOS:750-751); MN-E6-CD8-CD28-41BB-3z (SEQ ID NOS:303-304); MN-E6-CD4-CD28-41BB-3z (SEQ ID NOS:754-755); MN-E6scFv-Fc-8-41BB-CD3z (SEQ ID NOS:310-311); MN-E6scFv-IgD-Fc-8-41BB-CD3z (SEQ ID NOS:770-771); MN-E6scFv-FcH-8-41BB-CD3z (SEQ ID NOS:315-316); MN-E6scFv-IgD-FcH-8-41BB-CD3z (SEQ ID NOS:772-773); MN-E6scFv-Fc-4-41BB-CD3z (SEQ ID NOS:318-319); MN-E6scFv-FcH-4-41BB-CD3z (SEQ ID NOS:321-322); MN-E6scFv-IgD-8-41BB-CD3z (SEQ ID NOS:323-324); MN-E6scFv-IgD-4-41BB-CD3z (SEQ ID NOS:327-328); MN-E6scFv-X4-8-41BB-CD3z (SEQ ID NOS:330-331); MN-E6scFv-X4-4-41BB-CD3z (SEQ ID NOS:333-334); MN-E6scFv-8-4-41BB-CD3z (SEQ ID NOS:336-337), or any of the aforementioned CARs wherein the MN-E6 is replaced by MN-C2, MN-C3 or MN-C8; MN-C2-CD8-3z (SEQ ID NOS:606-607); MN-C2-CD4-3z (SEQ ID NOS:758-759); MN-C2-CD8-CD28-3z (SEQ ID NOS:608-609); MN-C2-CD4-CD28-3z (SEQ ID NOS:760-761); MN-C2-CD8-41BB-3z (SEQ ID NOS:610-611 and SEQ ID NOS:718-719); MN-C2-CD4-41BB-3z (SEQ ID NOS:762-763); MN-C2-CD8-CD28-41BB-3z (SEQ ID NOS:306-307); MN-C2-CD4-CD28-41BB-3z (SEQ ID NOS:766-767); MN-C2-Fc-8-41BB-CD3z (SEQ ID NOS:732-733); MN-C2-IgD-Fc-8-41BB-CD3z (SEQ ID NOS:734-735); MN-C2-FcH-8-41BB-CD3z (SEQ ID NOS:736-737); MN-C2-IgD-FcH-8-41BB-CD3z (SEQ ID NOS:738-739); MN-C2-IgD-8-41BB-CD3z (SEQ ID NOS:740-741); MN-C2-X4-8-41BB-CD3z (SEQ ID NOS:742-743). Another aspect of the invention is a method for treating a patient diagnosed with, suspected of having, or at risk of developing a cancer, wherein the patient is administered an effective amount of immune cells that have been transduced with one of the aforementioned CARs wherein the MN-E6 is replaced by a peptide comprising antibody variable domain fragments that are specific for a cancer antigen. In any of the above methods, the immune cell may be a T cell and may further be isolated from the patient to be treated.

Other MUC1 Cleavage Sites

It is known that MUC1 is cleaved to the growth factor receptor form, MUC1*, on some healthy cells in addition to cancer cells. For example, MUC1 is cleaved to MUC1* on healthy stem and progenitor cells. A large percentage of bone marrow cells are MUC1* positive. Portions of the intestine are MUC1* positive.

The inventors have discovered that MUC1 can be cleaved at different positions that are relatively close to each other but the location of cleavage changes the fold of the remaining portion of the extracellular domain. As a result, monoclonal antibodies can be identified that bind to MUC1* cleaved at a first position but do not bind to MUC1* that has been cleaved at a second position. This discovery is disclosed in WO2014/028668, filed Aug. 14, 2013, the contents of which are incorporated by reference herein its entirety. We identified a set of anti-MUC1* monoclonal antibodies that bind to MUC1* as it appears on cancer cells but do not bind to MUC1* as it appears on stem and progenitor cells. Conversely, we identified a second set of monoclonal antibodies that bind to stem and progenitor cells but do not bind to cancer cells. One method used to identify stem specific antibodies is as follows: supernatants from monoclonal hybridomas were separately adsorbed onto 2 multi-well plates. Stem cells, which are non-adherent cells, were put into one plate and cancer cells which are adherent were put into an identical plate. After an incubation period, the plates were rinsed and inverted. If the non-adherent stem cells stuck to the plate, then the monoclonal antibody in that particular well recognizes stem cells and will not recognize cancer cells. Antibodies that did not capture stem cells or antibodies that captured cancer cells were identified as cancer specific antibodies. FACS analysis has confirmed this method works.

Antibodies MN-E6 and MN-C2 are examples of cancer-specific antibodies. Antibodies MN-C3 and MN-C8 are examples of stem-specific antibodies. Although both sets of antibodies are able to bind to a peptide having the PSMGFR sequence, FACS analysis shows that the anti-MUC1* polyclonal antibody and MN-C3 bind to MUC1* positive bone marrow cells but MN-E6 does not. The MUC1* polyclonal antibody was generated by immunizing a rabbit with the PSMGFR peptide. Similarly, MN-C3 binds to stem cells of the intestinal crypts but MN-E6 does not. Conversely, MN-E6 antibody binds to cancerous tissue while the stem-specific MN-C3 does not. Competition ELISA experiments indicate that the C-terminal 10 amino acids of the PSMGFR peptide are required for MN-E6 and MN-C2 binding, but not for MN-C3 and MN-C8. Therefore, another method for identifying antibodies that are cancer specific is to immunize with a peptide having the sequence of the PSMGFR peptide minus the 10 N-terminal amino acids or use that peptide to screen for antibodies or antibody fragments that will be cancer specific. Antibodies that bind to a peptide with a sequence of PSMGFR peptide minus the N-terminal 10 amino acids but do not bind to a peptide with a sequence of PSMGFR peptide minus the C-terminal 10 amino acids are cancer specific antibodies for use in the treatment or prevention of cancers.

The extracellular domain of MUC1 is also cleaved on stem cells and some progenitor cells, where activation of cleaved MUC1 by ligands NME1 in dimer form or NME7 promotes growth and pluripotency and inhibits differentiation. The transmembrane portion of MUC1 that remains after cleavage is called MUC1* and the extracellular domain is comprised essentially of the Primary Sequence of MUC1 Growth Factor Receptor (PSMGFR) sequence. However, the exact site of cleavage can vary depending on cell type, tissue type, or which cleavage enzyme a particular person expresses or overexpresses. In addition to the cleavage site that we previously identified which leaves the transmembrane portion of MUC1* comprising most or all of the PSMGFR SEQ ID NO:2, other cleavage sites result in an extended MUC1* comprised of most or all of SNIKFRPGSVVVQLTLAFREGTINVHDVETQFNQYKTEAASRY (SEQ ID NO:620); or SVVVQLTLAFREGTINVHDVETQFNQYKTEAASRY (SEQ ID NO:621). The site of MUC1 cleavage affects how the remaining extracellular domain folds. We have identified monoclonal antibodies that bind to cleaved MUC1* on cancer cells but do not bind to cleaved MUC1* as it exists on healthy stem and progenitor cells.

Whereas an anti-MUC1* antibody or antibody-like molecule may be most effective if it competitively inhibits the binding of NME1, NME6, NME8 or NME7 or NME7-AB to MUC1*, for example an antibody that binds to the PSMGFR sequence especially if said antibody is unable to bind to a PSMGFR peptide if the 10 C-terminal amino acids are missing, antibodies or antibody-like molecules that carry a payload need not competitively inhibit the binding of MUC1* ligands to be effective as anti-cancer agents. For example antibodies or antibody-like molecules that are conjugated to a toxin could be effective at killing target cancer cells without necessarily inhibiting binding of the activating ligands. For example, antibodies or antibody-like molecules incorporated into CAR Ts or BiTEs which recruit the patient's immune system to the tumor can be effective as anti-cancer agents even if the antibody fragment targets a portion of MUC1* such that antibody fragment binding does not competitively inhibit the binding of NME1, NME6, NME8, NME7-AB or NME7. In a preferred embodiment the antibody fragment incorporated into a CAR, an adaptive T cell receptor or a BiTE competitively inhibits the binding of NME1, NME6, NME8, NME7-AB or NME7 to MUC1*.

Antibodies that are able to bind to the extracellular domain of the remaining transmembrane portion block the interaction between the MUC1* extracellular domain and activating ligands and in this way can be used as therapeutic agents, for example for the treatment of cancers. Anti-MUC1* antibodies are also useful for the growth, delivery, identification or isolation of stem cells both in vitro and in vivo.

General Strategy for Using Antibodies, Antibody Fragments and CARs that Target the Extracellular Domain of MUC1*

Monoclonal antibodies MN-C3 and MN-C8 have a greater binding affinity for blood cells than solid tumor cancer cells. Humanized antibodies and antibody fragments containing sequences derived from the variable regions of MN-C3 and MN-C8 can be used as a stand alone therapy or integrated into CAR Ts, BiTEs, ADCs for the treatment of blood cancers.

Alternatively, humanized antibodies and antibody fragments containing sequences derived from the variable regions of MN-C3 and MN-C8 can be used to deliver stem cells to a specific location such as for in situ human therapeutics. In one case, a substrate coated with humanized MN-C3 or MN-C8 derived antibodies or antibody fragments is loaded with stem cells then inserted into a patient. In another case, a substrate coated with humanized MN-C3 or MN-C8 derived antibodies or antibody fragments is inserted into a patient in order to recruit the patient's own stem cells to a specific area for therapy. Human therapies in which antibodies that bind to human stem cells will be of therapeutic use include spinal cord repair. Substrates coated with humanized MN-C3 or MN-C8 derived antibodies or antibody fragments are also used to identify or isolate human antibodies. Humanized MN-C3 or MN-C8 derived antibodies can also be used to stimulate the growth of stem cells.

CARs and Cleavage Enzymes

Many applications of CAR T therapy are limited by the length or flexibility of the extracellular domain between the T cell membrane and the antibody fragment that will direct the T cell to the desired location. For example, the surface of solid tumor cancer cells is populated with a myriad of cell surface proteins and growth factor receptors. Many of these cell surface proteins have bulky extracellular domains that limit the access of immune cells, such as T cells or CAR T cells, to the tumor cell surface. In one example, MUC1 and the cleaved growth factor receptor form MUC1* are overexpressed on over 75% of solid tumor cancers and on some blood cancers. The extracellular domain of MUC1 full-length contains between about 1,500 and 2,500 amino acids while the extracellular domain of MUC1* contains only about 45 to 65 amino acids. Variability in the length of MUC1 full-length is due to variability in the number of tandem repeat units that are expressed. Variability in the length of MUC1* is due to different cleavage sites when MUC1 is cleaved by different cleavage enzymes. Whereas it is most desirable to get the T cell close to the surface of the cancer cell, access can be sterically hindered by neighboring proteins, including full-length MUC1, that have large and bulky extracellular domains. This is especially true for early stage cancers. Tissue studies show that early stage cancers have more full-length MUC1 than late stage cancers that can be devoid of any full-length MUC1. This problem can in some cases severely limit the efficacy of cancer immunotherapies, including CAR T, adaptive T cell therapy, BiTEs and other T cell engagers.

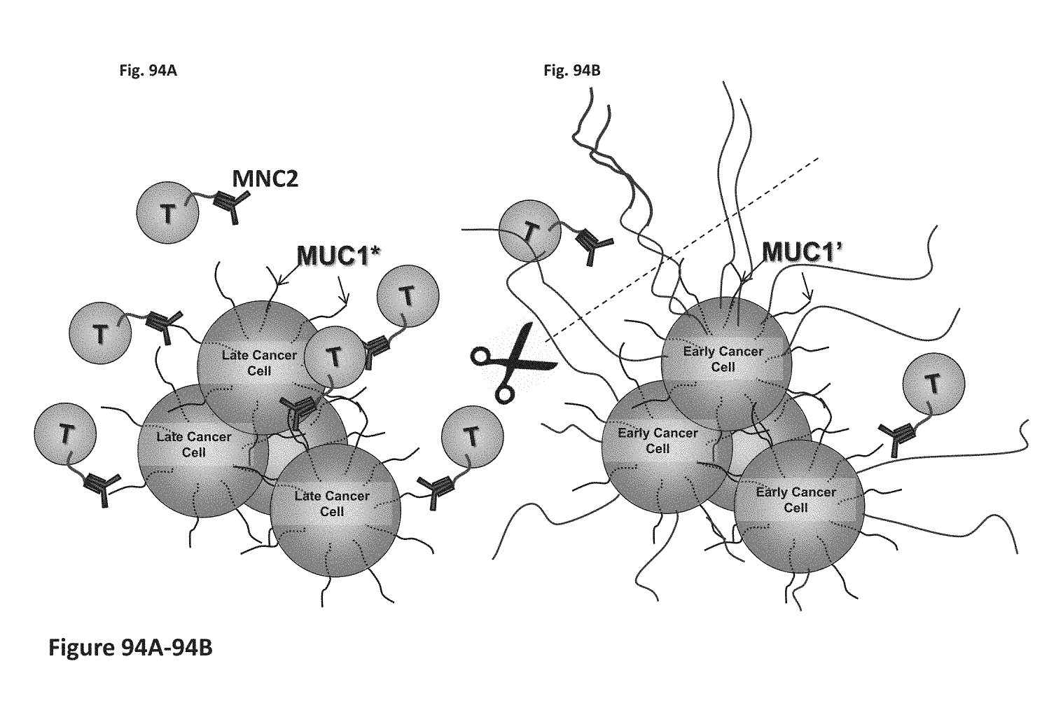

One solution to this problem is to express or activate cleavage enzymes in the area of the targeted tumor cells to cleave the bulky proteins that restrict access of T cells to the tumor. FIGS. 94A-94B show a cartoon of a CAR T cell that when near the tumor, expresses a cleavage enzyme that then cleaves MUC1 to MUC1*.

In one aspect of the invention, the cleavage enzyme and the CAR are transduced into the same T cell. In another aspect of the invention, the cleavage enzyme is on an inducible promoter such that its expression is activated when the CAR engages the targeted cancer cells. In some cases, the expression of the cleavage enzyme is controlled by an inducible promoter. In one aspect of the invention, expression of the cleavage enzyme is induced when the immune cell is activated, for example when it recognizes or engages its target. In one example, a T cell is transfected or transduced with a cleavage enzyme whose expression is induced when the T cell recognizes a target cancer cell. One way to do this is to induce expression of the cleavage enzyme when, or shortly after, an NFAT protein is expressed or translocated to the nucleus. For example, a sequence derived from an NFAT promoter region is put upstream of the gene for the cleavage enzyme. In this way, when the transcription factors that bind to the promoter of the NFAT protein are present in sufficient concentration to bind to and induce transcription of the NFAT protein, they will also bind to that same promoter that is engineered in front of the sequence for transcription of the cleavage enzyme. The NFAT protein may be NFAT1 also known as NFATc2, NFAT2 also known as NFATc or NFATc1, NFAT3 also known as NFATc4, NFAT4 also known as NFATc3, or NFAT5. In one aspect of the invention, the NFAT is NFATc1, NFATc3 or NFATc2. In one aspect of the invention, the NFAT is NFAT2 also known as NFATc1. SEQ ID NO:646 shows nucleic acid sequence of the upstream transcriptional regulatory region for NFAT2. The promoter sequence for NFAT gene may include the nucleic acid sequence of SEQ ID NO:781-783 or SEQ ID NO:815 as examples, but it can be seen that the optimal sequence or minimal sequence for expression of the cleavage enzyme may be obtained by making fragments, extensions or mutations of the promoter and testing for the strength of the promoter with respect to expression of the cleavage enzyme. In one aspect of the invention, the transcriptional regulatory region for NFAT2 is engineered upstream of the gene encoding the cleavage enzyme MMP9 (SEQ ID NO:647) or the catalytic sub-unit of MMP9 (SEQ ID NO:648). In one aspect of the invention, the NFAT is NFATc3 and the promoter sequence of NFATc3 includes nucleic acid sequences from SEQ ID NO:816. In one aspect of the invention, the transcriptional regulatory region for NFATc3 is engineered upstream of the gene encoding the cleavage enzyme MMP9 (SEQ ID NO:647) or the catalytic sub-unit of MMP9 (SEQ ID NO:648). In another aspect of the invention, the NFAT is NFATc2. SEQ ID NO:817-818 shows nucleic acid sequence of the upstream transcriptional regulatory region for NFATc2. In one aspect of the invention, the transcriptional regulatory region for NFATc2 is engineered upstream of the gene encoding the cleavage enzyme MMP9 (SEQ ID NO:647) or the catalytic sub-unit of MMP9 (SEQ ID NO:648).

Another method for having the expression of the cleavage enzyme induced when the T cell or CAR T cell is activated is to have the gene for the cleavage enzyme on an inducible promoter where the NFAT protein itself binds to and induces transcription of the cleavage enzyme. In this case, an NFAT response element (NFAT RE) may be positioned upstream of the gene for the cleavage enzyme or fragment of the cleavage enzyme. The NFAT may bind to its responsive element upstream of the cleavage enzyme alone or as part of a complex. The NFAT protein may be NFATc1, NFATc2, NFATc3, NFATc4, or NFAT5. In a preferred embodiment, the NFAT protein is NFAT2 aka NFATc1, aka NFATc. The gene of the cleavage enzyme or fragment thereof is cloned downstream of an NFAT-response element (SEQ ID NO:649), which may be repeats of the response element (SEQ ID NO:650) and CMV minimal promoter (mCMV) (SEQ ID NO:651) to induce expression of cleavage enzyme by NFAT protein. The NFAT response element may include nucleic acid sequence of NFAT consensus sequence (SEQ ID NO:804). The NFAT response element may include the nucleic acid sequence of SEQ ID NOS:805-814 as examples, but it can be seen that the optimal sequence or minimal sequence for expression of the cleavage enzyme may be obtained by making fragments, extensions or mutations of the responsive element nucleic acid and testing for the strength of the responsive element with respect to expression of the cleavage enzyme. The enhancer region of Foxp3 also contains NFAT response elements within the 120-bp from 2079 to 2098 (SEQ ID NO:821). The NFAT response element may include nucleic acid NFAT consensus sequence of (5′-cattttttccat-3′) (SEQ ID NO:819) or (5′-tttttcca-3′) (SEQ ID NO:820), which NFATc1 specifically binds to (Xu et al., Closely related T-memory stem cells correlate with in vivo expansion of CAR. CD19-T cells and are preserved by IL-7 and IL-15, Blood 2014 123:3750-3759), or repeats thereof. The NFAT response elements may also be separated by nucleic acid spacer sequences. Other NFAT responsive elements may exist and may further be discovered, and a skilled artisan in the art when directed to determine NFAT responsive element may do so by carrying out molecular biological assays to obtain it given the guidance of at least the responsive elements as set forth as SEQ ID NOS: 804-814 albeit as only mere examples. In one aspect of the invention, the cleavage enzyme that is downstream of the NFAT-response element and CMV minimal promoter is MMP9 (SEQ ID NO:652). In another aspect of the invention, the cleavage enzyme is a catalytic sub-unit of MMP9 (SEQ ID NO:653).

Because NFATs 1-4 are regulated by the calcineurin pathway, potential toxicities that may arise in a patient can be stopped by treatment with an immunosuppressive agent such as FK506, Cyclosporin, Cyclosporin A, or Tacrolimus that block calcineurin activity and inhibit NFAT translocation to the nucleus. The T cell transduced or transfected with a cleavage enzyme on an inducible promoter may also be transfected or transduced with a CAR that recognizes a protein or molecule on the cancer cell. In a specific example, the cleavage enzyme is one that is able to cleave MUC1 full-length and the CAR bears an antibody fragment that directs it to MUC1* on the surface of cancer cells.

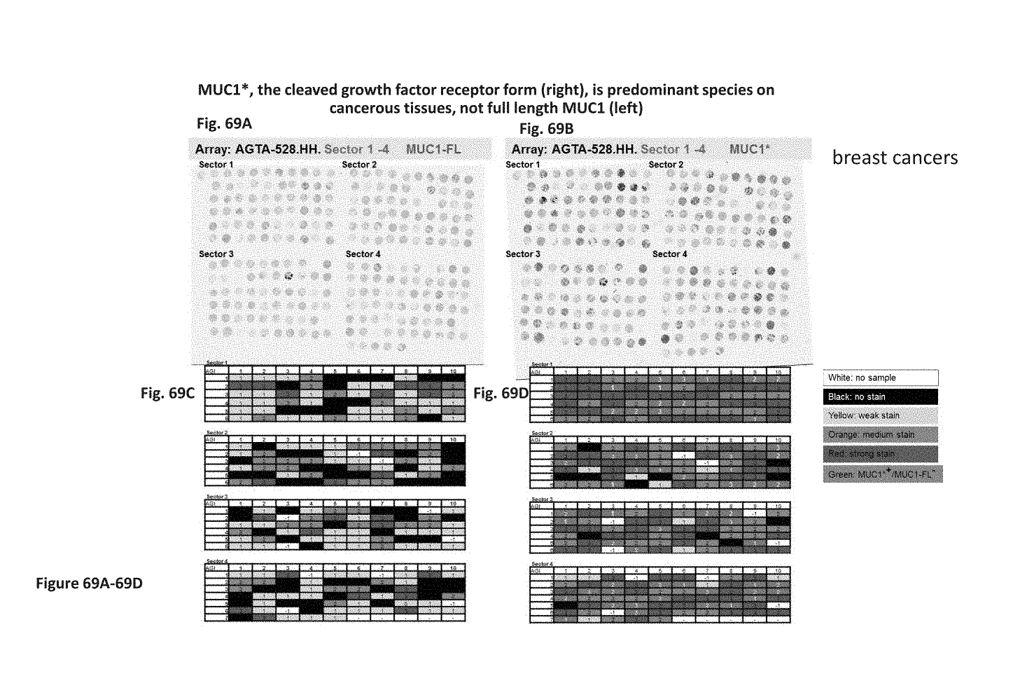

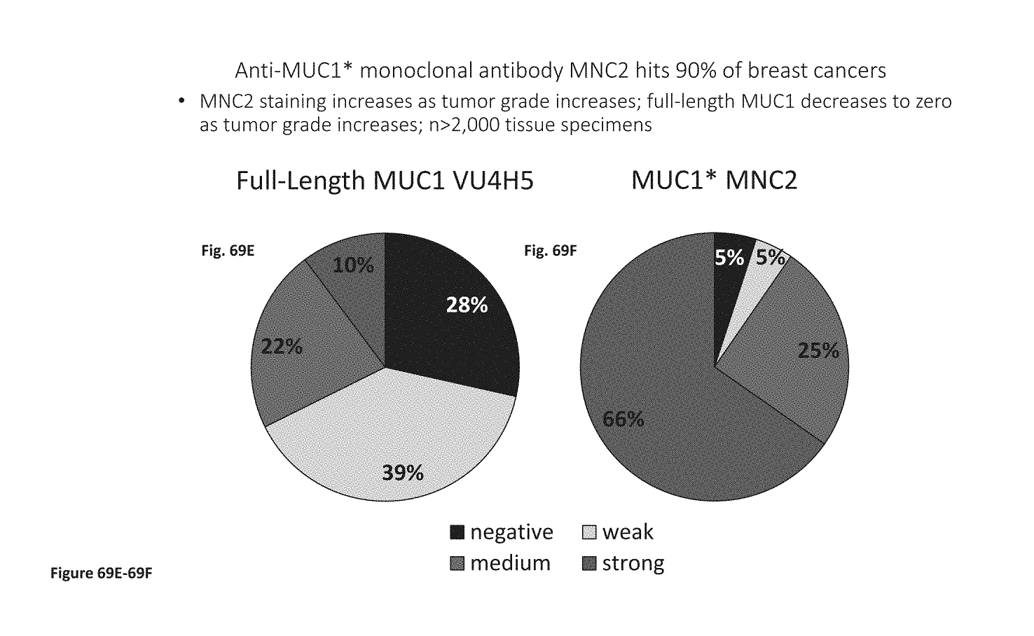

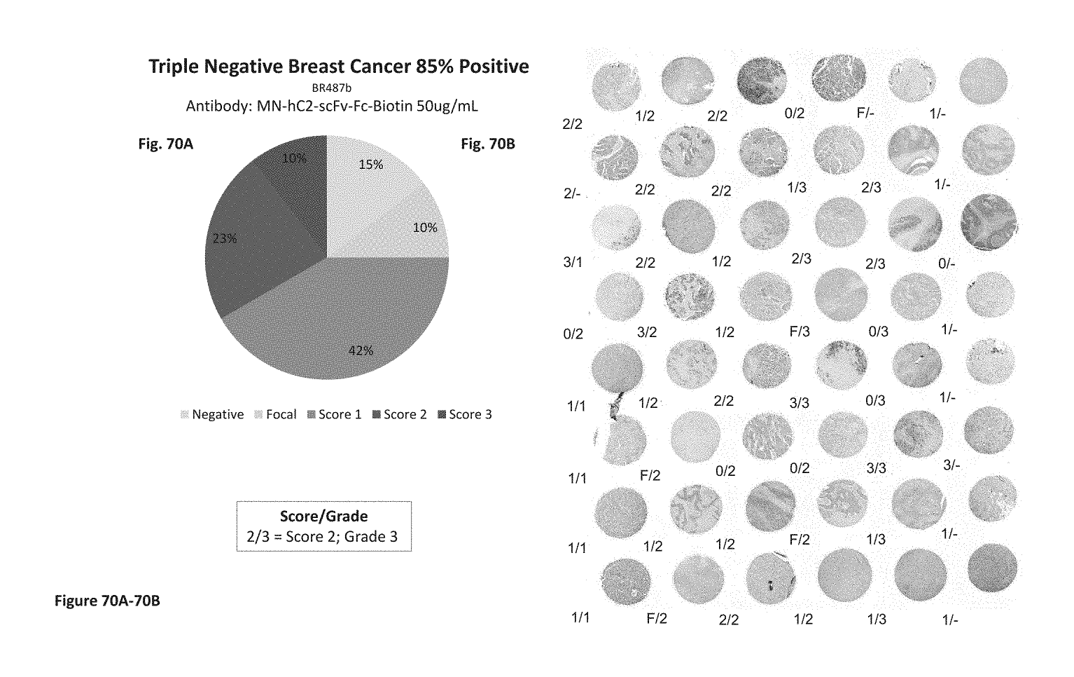

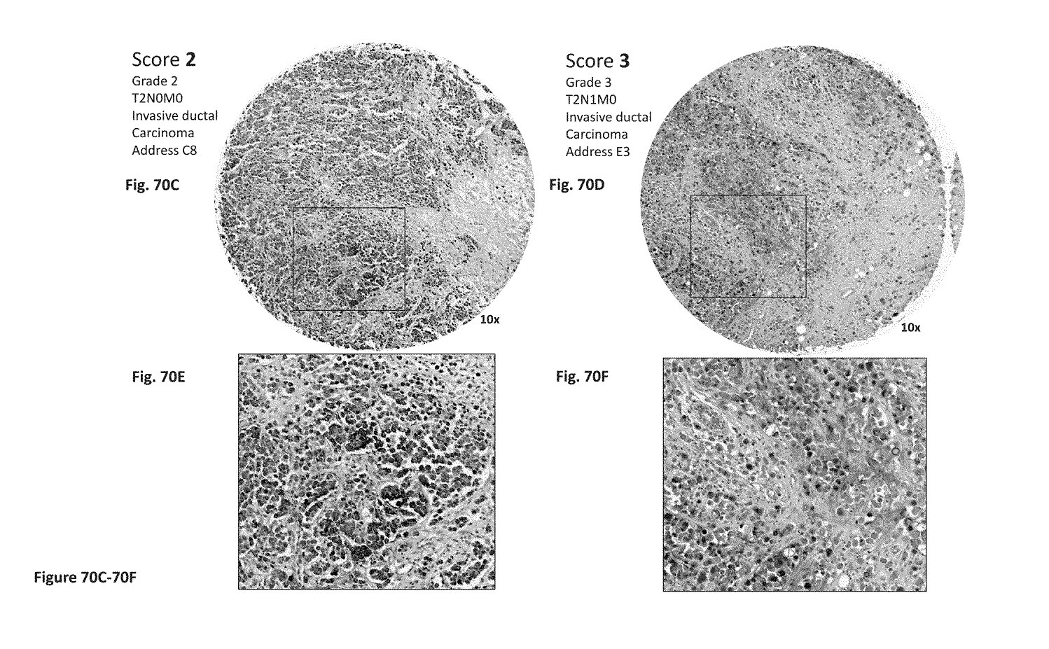

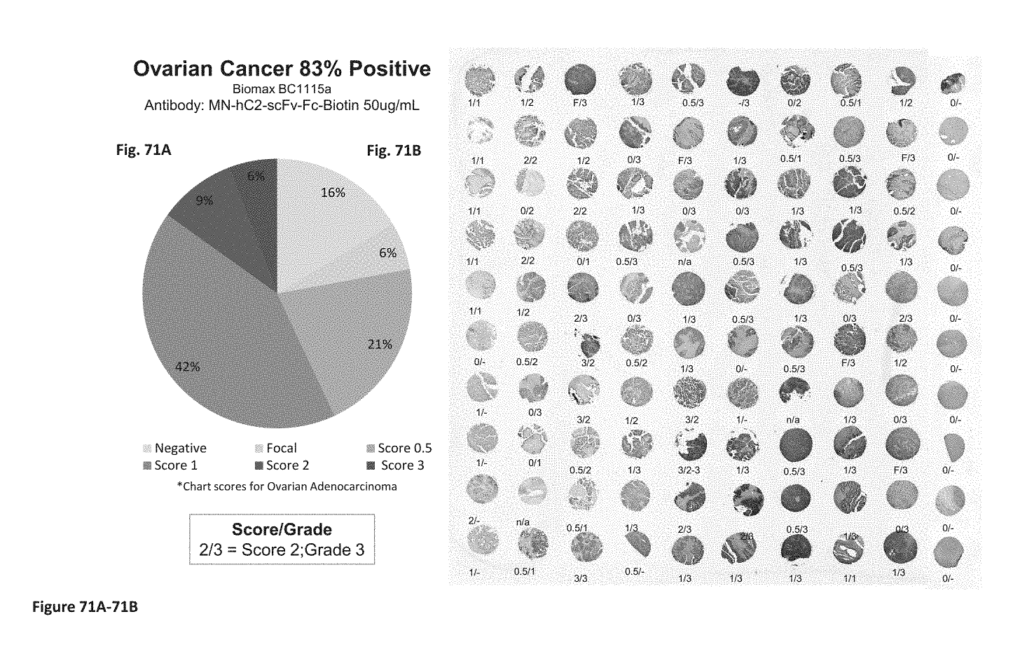

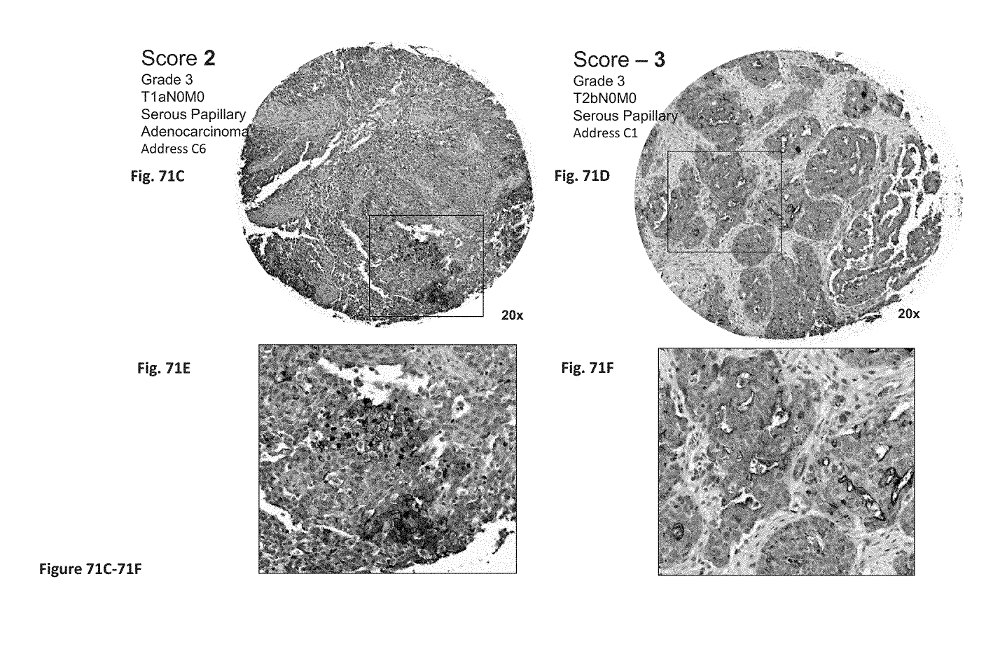

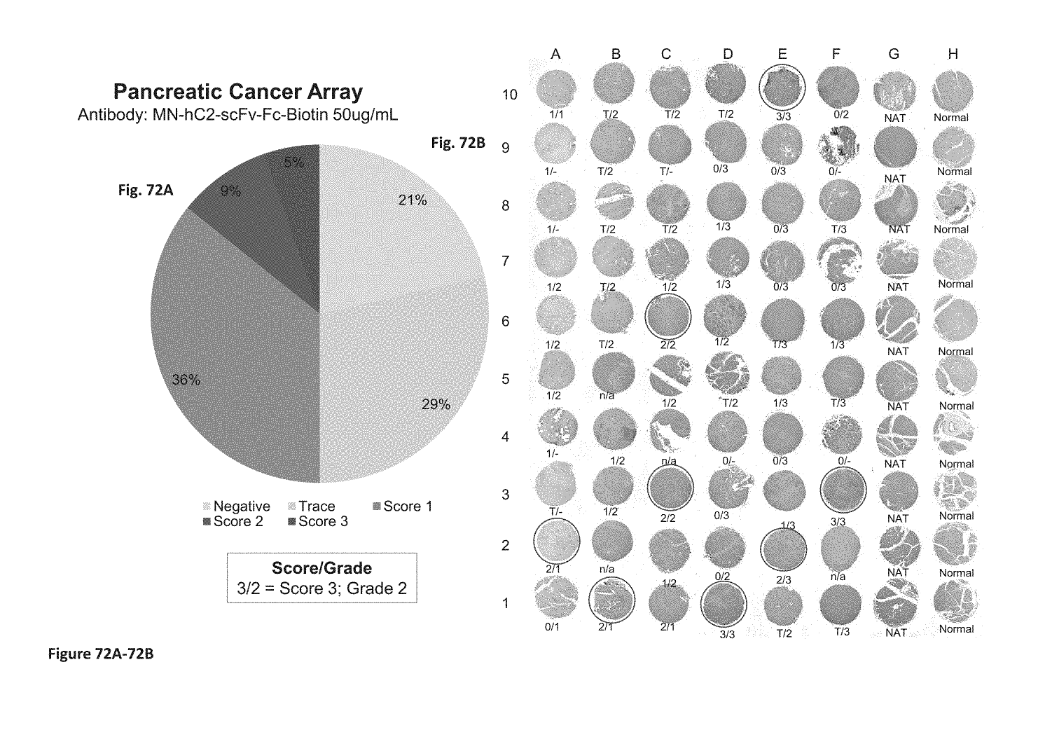

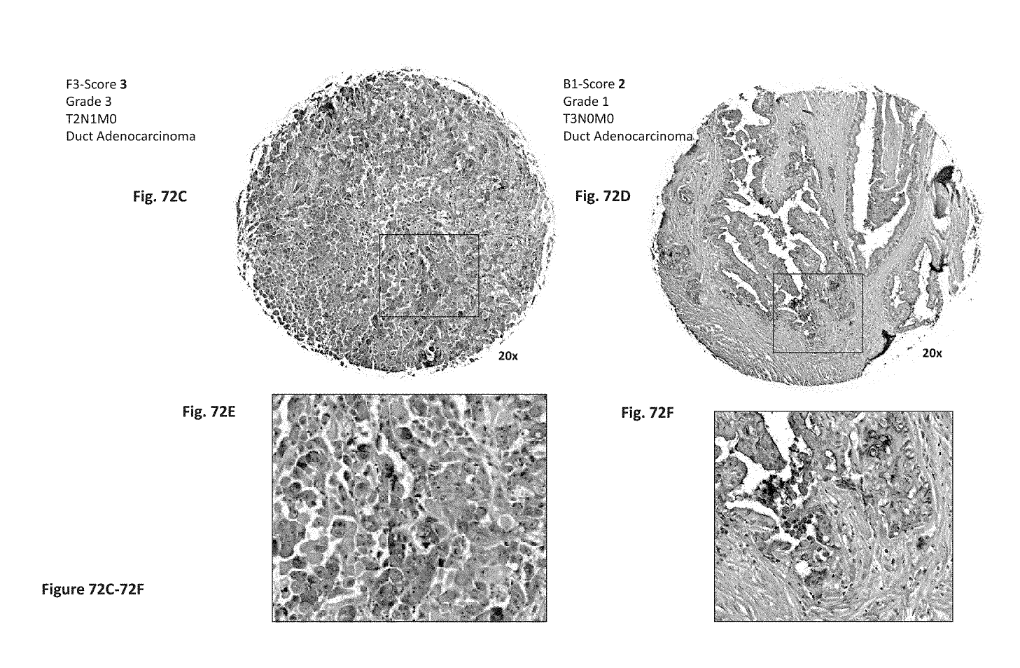

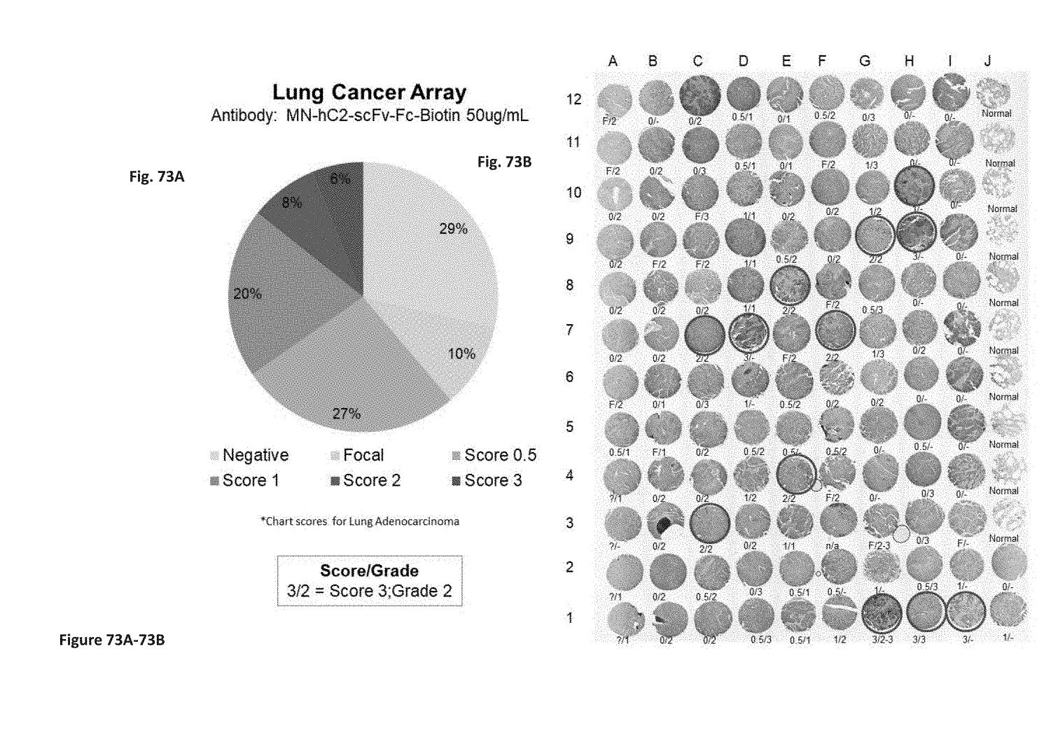

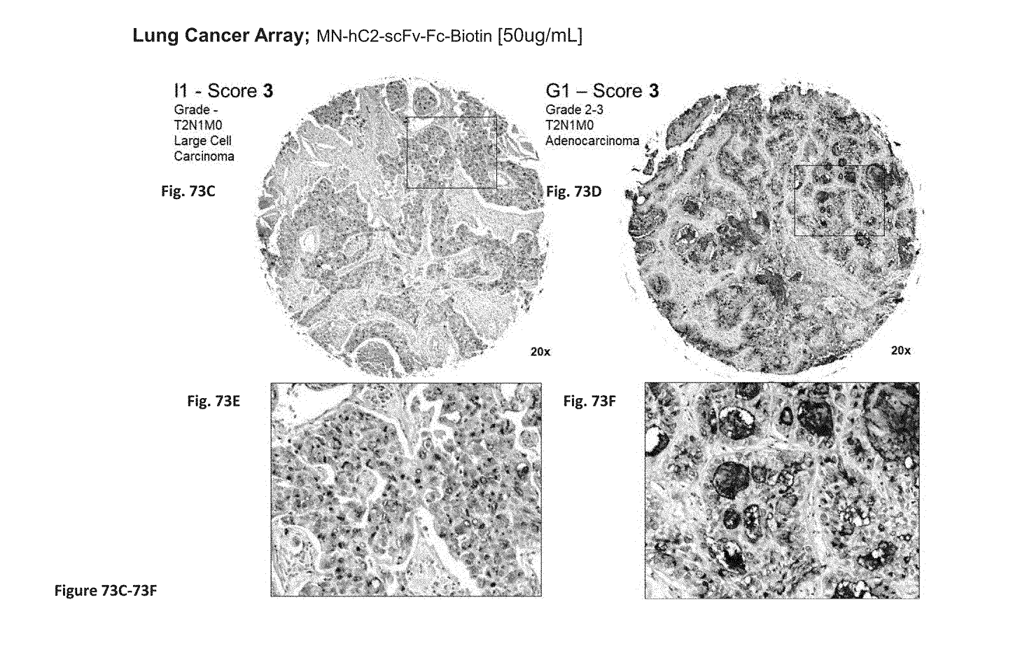

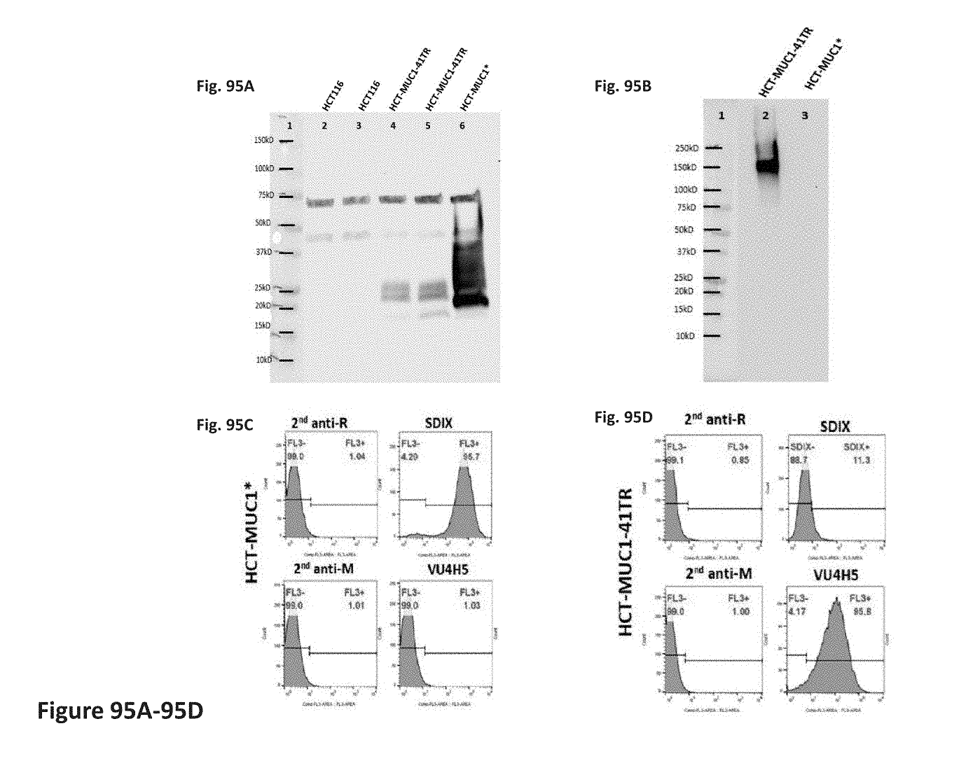

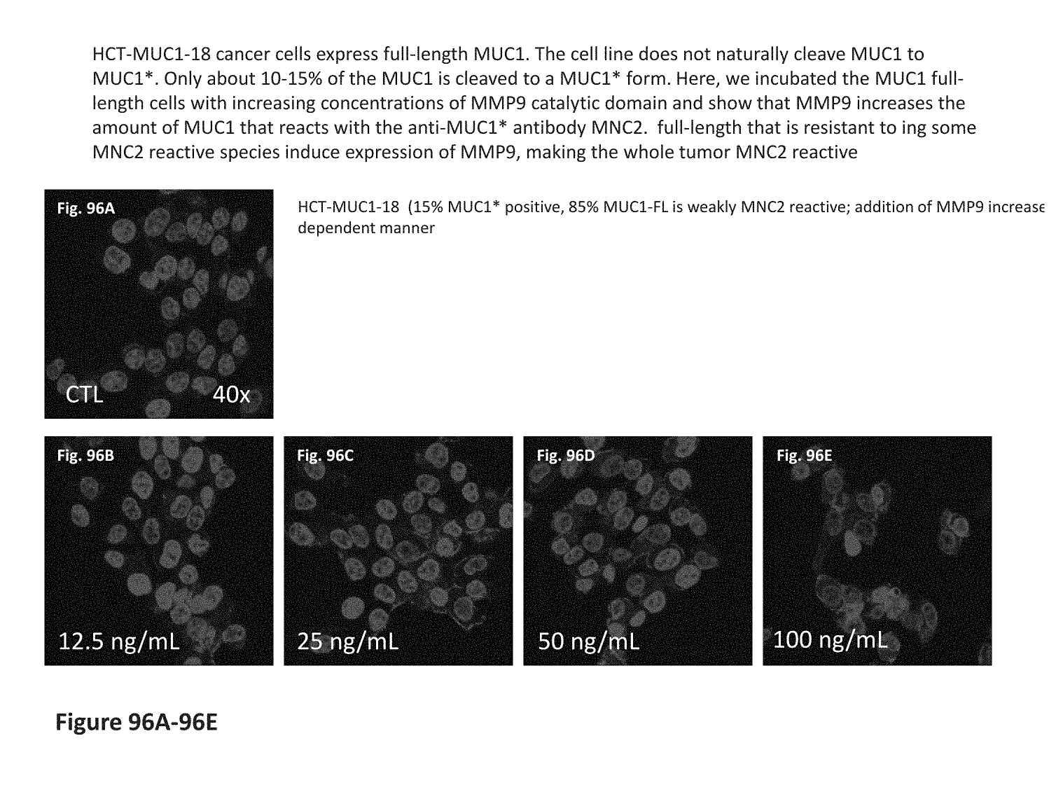

To determine which cleavage enzymes cleave MUC1 on cancer cells, we tested a series of MMP and ADAM enzyme inhibitors. These experiments pointed to MMP9 as being an important cleavage enzyme in cancer cells. To confirm that MMP9 cleaves MUC1 on cancer cells, we transfected HCT-116 MUC1 negative colon cancer cells with a mimic of full-length MUC1 having 41 tandem repeat domains: HCT-MUC1-41TR. Through single cell cloning we were able to establish this cell line wherein MUC1 only minimally gets cleaved to MUC1*. FIGS. 95A-95D show Western blots and FACS analysis showing that HCT-MUC1-41TR is 95% positive for full-length MUC1 and only 5-10% positive for the cleaved form, MUC1*. HCT-MUC1-41TR cells were incubated with MMP9 at varying concentrations and then assayed by immunofluorescence to measure binding of MNC2 monoclonal antibody to the resultant cells. As can be seen in FIGS. 96A-96E, binding of MNC2 increased as the concentration of MMP9 added to the cells increased. These experiments show that MMP9 cleaves MUC1 to a form that is recognized by MNC2. The human cancer tissue array studies we performed (FIG. 69A-69D, FIG. 70A-70F, FIG. 71A-71F, FIG. 72A-72F, FIG. 73A-73F) show that MNC2 recognizes the form of cleaved MUC1 that is present on cancerous tissue but not on healthy cells or tissues (FIG. 74A-74I). Importantly, MNC2 does not recognize the form of cleaved MUC1 that is expressed on healthy hematopoietic stem cells of the bone marrow.

In one aspect of the invention, an immune cell is transduced with both a CAR to target the immune cell to the tumor, and a cleavage enzyme. The CAR and the cleavage enzyme can be encoded on the same plasmid or on two different plasmids. In one aspect, the cleavage enzyme is on an inducible promoter. In another aspect, expression of the cleavage enzyme is induced by a protein that is expressed when the immune cell is activated. In one case, expression of the cleavage enzyme is induced by an NFAT protein. In another aspect, expression of the cleavage enzyme is induced by NFATc1. In another aspect, expression of the cleavage enzyme is induced when one of the NFAT proteins binds to an NFAT response element that is inserted upstream of the gene for the cleavage enzyme or a catalytically active fragment thereof. In one aspect, the cleavage enzyme is MMP9 or a fragment of MMP9 that is catalytically active.

In one aspect of the invention, the cleavage enzyme is MMP9 (SEQ ID NO:643). Some cleavage enzymes are naturally expressed as pro-enzymes that need to be activated. This can be accomplished by biochemical means, by expressing a co-enzyme that activates a cleavage enzyme or by engineering the enzyme in an activated form. The invention anticipates overcoming this problem by co-expressing the cleavage enzyme with its activator. In one aspect of the invention, the cleavage enzyme is MMP9 and the co-activator is MMP3. In another aspect of the invention, the cleavage enzyme is expressed in a form that is already active, for example by expressing a fragment of the cleavage enzyme that still has catalytic function. In one case, the cleavage enzyme is an MMP9 fragment that is catalytically active. One example of an MMP9 catalytic fragment is given as SEQ ID NO:645.

MMP9, which must be activated by MMP3, is overexpressed in a large percentage of solid tumors. Further, it is known that MNC2 anti-MUC1* monoclonal antibody recognizes MUC1 after it is cleaved by MMP9. The breast, ovarian, pancreatic and lung cancer arrays that were shown in FIGS. 69-73 were probed with MNC2-scFv, further indicating that MUC1 in these cancers is being cleaved by MMP9. To see if cleavage of tumors by MMP9 would increase T cell access to the tumor, we did a series of experiments using a cell line that expresses full-length MUC1, HCT-MUC1-41TR, a breast cancer cell line that is a high expresser of both full-length MUC1 and MUC1* and a MUC1 negative cell line that we transfect with MUC1*45. We transfected cells with MMP9 and MMP3, which activates MMP9. We took the supernatant of those cells, which contained activated MMP9, and added it to the various cells, which were then co-cultured with T cells transduced with an anti-MUC1* CAR: huMNC2-CAR44. The result was greatly increased CAR T cell killing of the targeted MUC1/MUC1* positive cancer cells, compared to the control cells that were not incubated with a MUC1 cleavage enzyme.

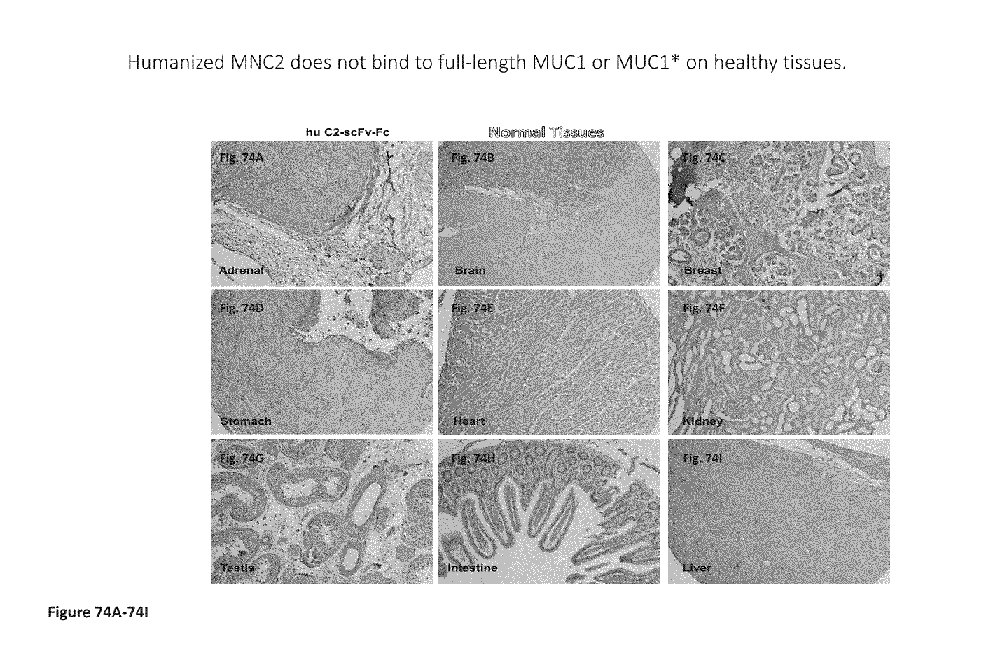

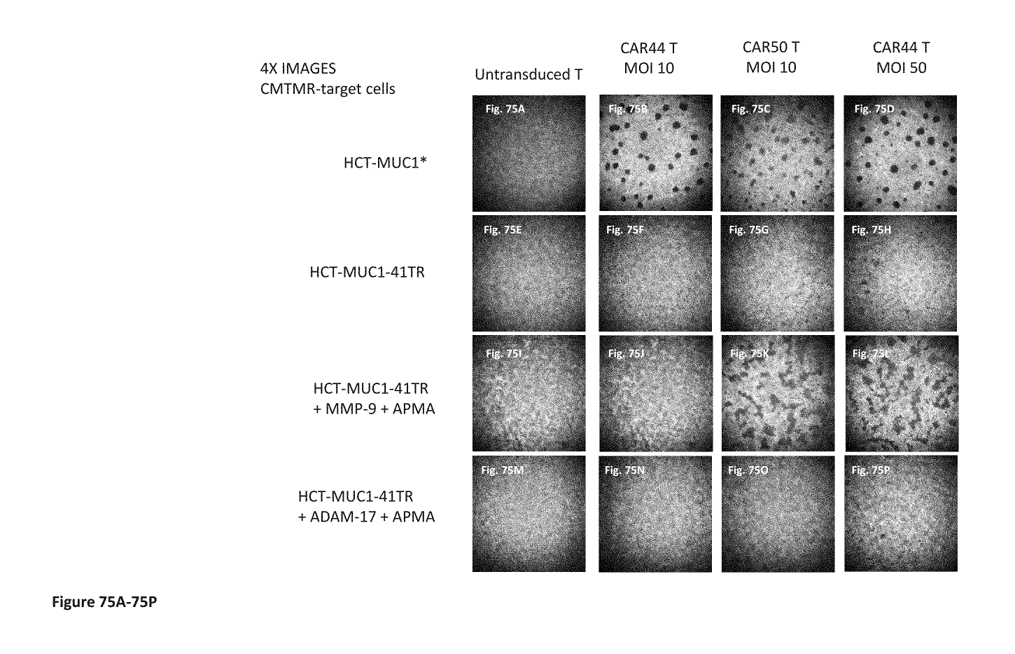

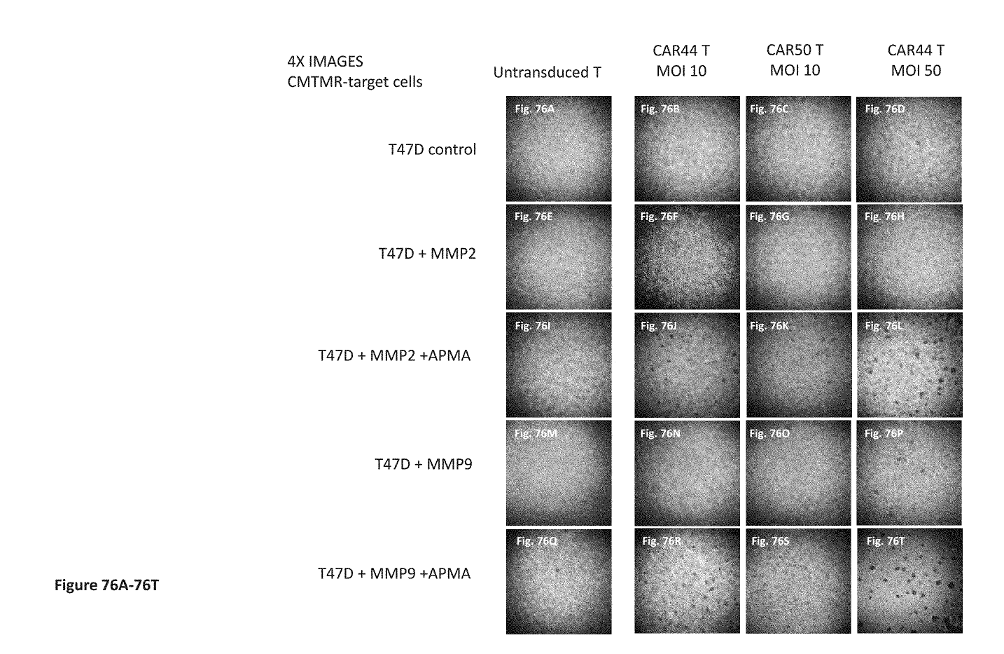

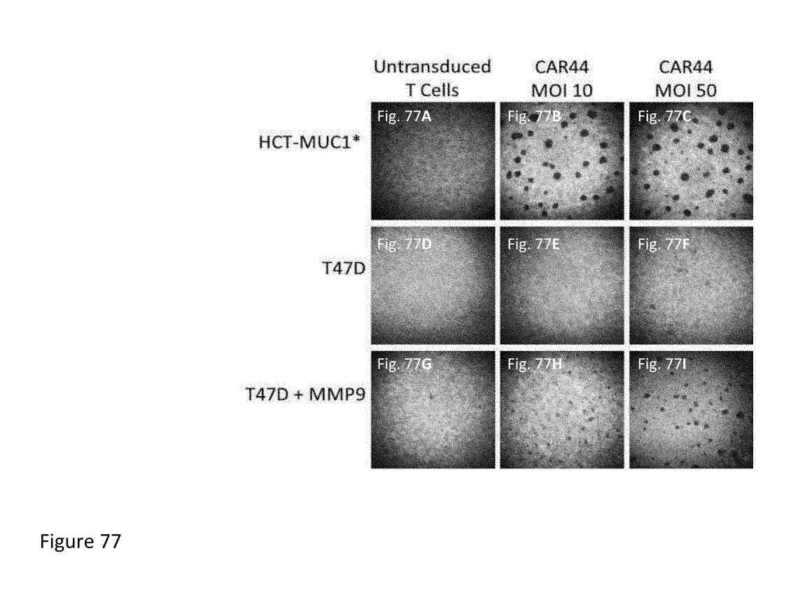

APMA is a biochemical that activates MMPs. We used APMA along with the conditioned media of cells that we transfected with either MMP9 or ADAM17 to see if any of these cleavage enzymes would cleave MUC1 on the HCT-MUC1-41TR cell line that only expresses full-length MUC1. As controls, we also tested the enzymes on HCT-MUC1* cells. The MUC1 and MUC1* expressing cells were stained with a red dye, CMTMR. Human T cells that were transduced with an anti-MUC1* CARs, CAR44 or CAR50 were co-cultured with the cancer cells. Untransduced T cells were used as a control. As can be seen in FIGS. 75B, 75C, and 75D, the anti-MUC1* CAR T cells effectively recognized and clustered the HCT-MUC1* cancer cells, which is a sign of T cell activation and killing. However, no CAR T cell induced clustering is visible in the wells containing HCT-MUC1-41TR, the full-length MUC1 expressing cells (FIGS. 75F, 75G, and 75H). However, the cells that were incubated with activated MMP9 show dramatic increase in CAR T cell induced clustering (FIGS. 75J, 75K, and 75I), implying that MMP9 cleaved the full-length MUC1 to a form of MUC1* that is recognized by MNC2 monoclonal antibody and more specifically by huMNC2-scFv. ADAM17 had no apparent effect. ADAM17 either did not cleave MUC1 or cleaved it at a position that is not recognized by MNC2, which is more likely.

We performed the same experiment, this time using T47D breast cancer cells that were hard to kill using anti-MUC1* CAR T cells presumably because they express high levels of full-length MUC1 as well as MUC1*. As can be seen in FIGS. 76B, 76C, and 76D, anti-MUC1* CAR44 and CAR50 have little effect on the T47D cancer cells. Only in FIG. 76D, which is CAR44 at the highest level of CAR expression in the T cells, do we see a small amount of CAR T cell induced clustering. However, the presence of activated MMP2 (FIGS. 76J, 76K, 76L) or activated MMP9 (FIGS. 76R, 76S, 76T) shows a dramatic increase in CAR T cell recognition, clustering and killing, showing that cleavage of full-length MUC1 increases T cell access to the cancer cells.

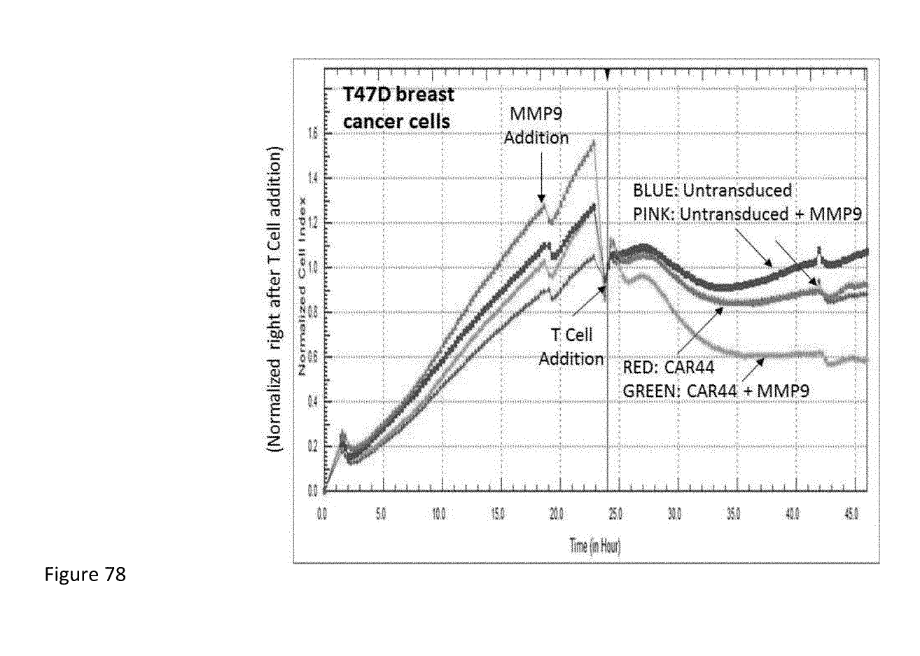

In another example, T47D MUC1 positive tumor cells were incubated with a recombinant catalytic domain of MMP9 (Enzo Life Sciences, Inc., Farmingdale, N.Y.) at either 100 ng/mL or 500 ng/mL. Western blot analysis showed that the MUC1/MUC1* positive cancer cells underwent extensive cleavage of MUC1 to MUC1*. In another example, T47D breast cancer cells were pre-incubated with a human recombinant MMP9 catalytic domain protein then co-cultured with anti-MUC1* CAR44 T cells. The specific killing of the T47D cells by CAR44 T cells was monitored in real-time on an xCelligence instrument that measures impedance as a function of time. This analysis uses electrode arrays upon which cancer cells are plated. The adherent cancer cells insulate the electrode and cause an increase in impedance as they grow. Conversely, T cells are not adherent and remain in suspension so do not increase or decrease impedance. However, if the T cells or CAR T cells kill the cancer cells on the electrode plate, the cancer cells ball up and float as they die, which causes the impedance to decrease. The addition of MMP9 catalytic domain dramatically increased the killing of T47D cancer cells. FIG. 78 shows an xCelligence graph of T47D breast cancer cells in co-culture with either untransduced T cells, as a control, or huMNC2-CAR44 T cells over a 45 hour period. After 18 hours of cancer cell growth, a catalytic sub-unit MMP9 was added to some of the cells. At 25 hours, T cells were added. As can be seen, huMNC2-CAR44 T cell killing is greatly improved when the T47D cells are pre-incubated with cleavage enzyme MMP9. In the xCelligence system, target cancer cells, which are adherent, are plated onto electrode array plates. Adherent cells insulate the electrode and increase the impedance. The number of adherent cancer cells is directly proportional to impedance. T cells are not adherent and do not contribute to impedance. Therefore, increasing impedance reflects growth of cancer cells and decreasing impedance reflects killing of cancer cells. Prostate cancer cell line DU145 expresses both MUC1 and MUC1* but at a much lower level of expression than T47D cells. DU145 cells are efficiently killed by anti-MUC1* CAR T cells in the presence or absence of a cleavage enzyme.

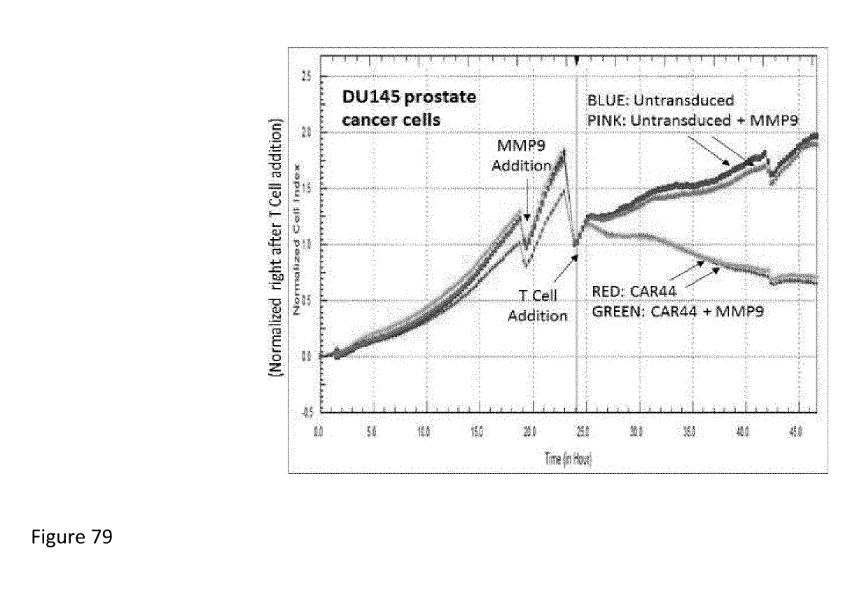

FIG. 79 shows an xCelligence graph of DU145 prostate cancer cells in co-culture with either untransduced T cells, as a control, or huMNC2-CAR44 T cells over a 45 hour period. After 18 hours of cancer cell growth, a catalytic sub-unit MMP9 was added to some of the cells. At 25 hours, T cells were added. As can be seen, huMNC2-CAR44 T cell killing of low density MUC1/MUC1* positive cancer cells is not affected by pre-incubation with cleavage enzyme MMP9. DU145 cancer cells express a significantly lower amount of MUC1 which includes the full-length form as well as MUC1*. The lower density of full-length MUC1 does not sterically hinder T cell access to the membrane proximal MUC1*. DU145 cells represent an early stage cancer that expresses both full length and cleaved MUC1 but at lower levels so that T cell access is not sterically hindered. T47D cells represent mid-stage cancers that express high levels of both MUC1 and MUC1*, wherein the density of MUC1 full-length sterically hinders access of T cells to the tumor. HCT-MUC1* cells are a MUC1 negative cell line that has been stably transfected with MUC1*₄₅, and they represent late stage cancer cells. It is significant that MUC1 cleaved to MUC1* by MMP9 is recognized by the anti-MUC1* antibody MNC2, which is the targeting head of the CAR. Immune cell access to tumor antigens on the cancer cell surface can be sterically hindered by the presence of bulky extra cellular domain proteins or other obstructing elements also known as the tumor micro-environment. The aforementioned serve as an example that can be extended to improve the efficacy of CAR T therapies that target other tumor antigens. In one aspect of the invention, an immune cell is transfected or transduced with both a CAR comprising an antibody fragment that targets a tumor antigen and a cleavage enzyme. In another aspect of the invention, an immune cell is transfected or transduced with both a CAR comprising an antibody fragment that targets a tumor antigen and a cleavage enzyme that cleaves a tumor antigen to a form recognized by the antibody fragment of the CAR. In one aspect, an immune cell is transfected or transduced with both a CAR comprising an antibody fragment that targets a tumor antigen and a cleavage enzyme that cleaves a tumor antigen to a form recognized by the antibody fragment of the CAR, wherein the antibody fragment of the CAR recognizes MUC1* extra cellular domain and the cleavage enzyme cleaves MUC1 to MUC1*. In one aspect, an immune cell, which may be a T cell or an NK cell, is transfected or transduced with a CAR comprising an antibody fragment derived from MNC2, MNE6, MNC3 or MNC8 and a cleavage enzyme chosen from the group comprising MMP1, MMP2, MMP3, MMP7, MMP8, MMP9, MMP11, MMP12, MMP13, MMP14, MMP16, ADAM9, ADAM10, ADAM17, ADAM 19, ADAMTS16, ADAM28 or a catalytically active fragment thereof.

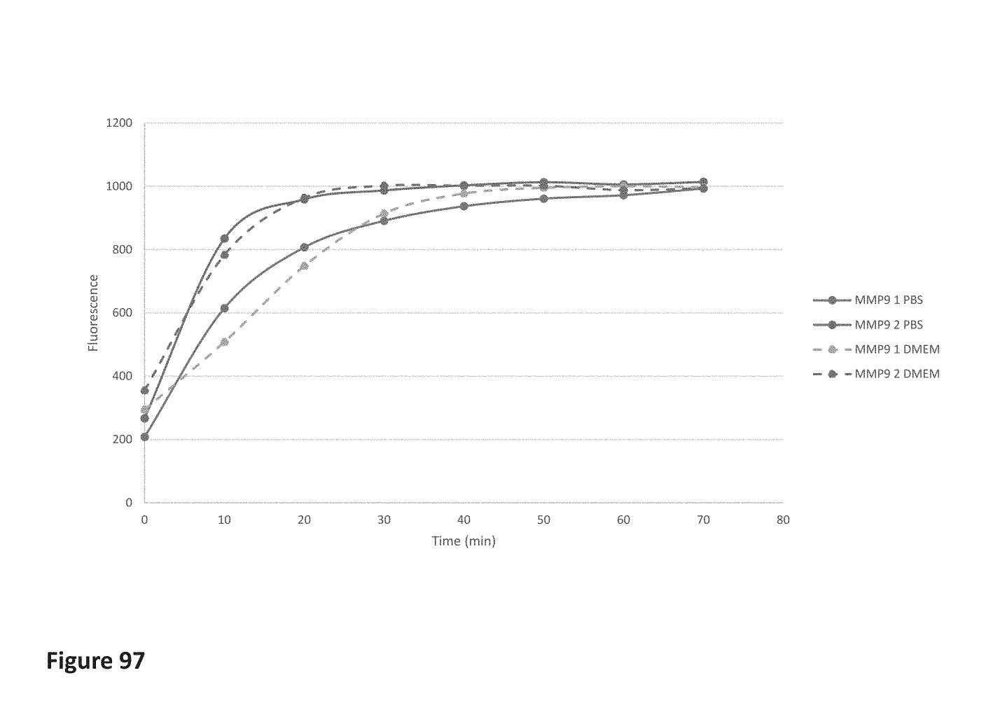

A convenient method for testing the presence of MMP9 is with a fluorogenic assay, for example using the OMNIMMP peptide assay kit. The kits have a peptide that is an MMP9 substrate that has been derivatized with a masked fluorophore. When MMP9 is added to a solution containing the peptide, MMP9 cleaves the peptide at a position that unmasks the fluorophore and the fluorescence can be read on a plate reader. MMP-9 activity is read in Relative Fluorescent Units (RFUs) which is an arbitrary value related to the amount of light detected by a plate reader set to excite each well containing samples at 328 nm and measure the emission at 393 nm. An increase in RFUs indicates cleavage of the Gly-Leu bond, unmasking of the fluorophore and therefore the presence of MMP-9. The sequence of the OMNIMMP peptide is Mca-Pro-Leu-Gly-Leu-Dpa-Ala-Arg-NH₂. AcOH [Mca=(7-methoxycoumarin-4-yl)acetyl; Dpa=N-3-(2,4-dinitrophenyl)-L-α,β-diaminopropionyl]. FIG. 97 shows a graph of the OMNIMMP fluorogenic peptide substrate of MMP9 being cleaved by MMP9 catalytic domain and emitting fluorescence. The MMP9 catalytic domain was added at two concentrations in either PBS, solid trace, or cell culture media, dashed trace. This experiment shows that the OMNIMMP peptide assay will measure the activity of MMP9 that has been secreted by cells even if they are in cell growth media.

A method for studying activation of the NFAT pathway is by chemically activating the pathway using PMA with lonomycin (Lyakh et al., Expression of NFAT-Family proteins in normal human T cells, MOLECULAR AND CELLULAR BIOLOGY, Vol. 17, No. 5, May 1997, p. 2475-2484; Rao et al., Transcription factors of the NFAT family—Regulation and function, Annu. Rev. Immunol. 1997. 15:707-47; Macian, NFAT proteins—Key regulators of T-cell development and function, Nature Reviews Immunology, Vol. 5, pp 472-484 June (2005)). It has been demonstrated that PMA and lonomycin induce expression of NFAT proteins. The above-cited references show a scheme of the regulation of NFAT activation. Ionomycin increases calcium which activates the Calcineurin/Calmodulin complex. Calcineurin/Calmodulin dephosphorylate NFAT, which causes NFATs, especially NFATc1, to be translocated to the nucleus where it binds to DNA to stimulate transcription of target genes. NFATc1 is one of the first NFAT proteins to be translocated to the nucleus upon T cell activation and it is only there transiently before it exits the nucleus. Therefore, PMA plus lonomycin activation of cells we transfected or transduced with NFAT inducible cleavage enzymes is physiologically relevant and mimics in vivo T cell activation turning on expression of the NFAT inducible cleavage enzymes described herein.

The HEK293T cell line (human embryonic kidney cell), originally referred as 293tsA1609neo, is a highly transfectable derivative of human embryonic kidney 293 cells, and contains the SV40 T-antigen. This cell line is competent to replicate vectors carrying the SV40 region of replication. It gives high titers when used to produce retroviruses. It has been widely used for retroviral production, gene expression and protein production. HEK293T cells were used in some of the early experiments, before the plasmids were inserted into lenti viral vectors and transduced into human T cells.

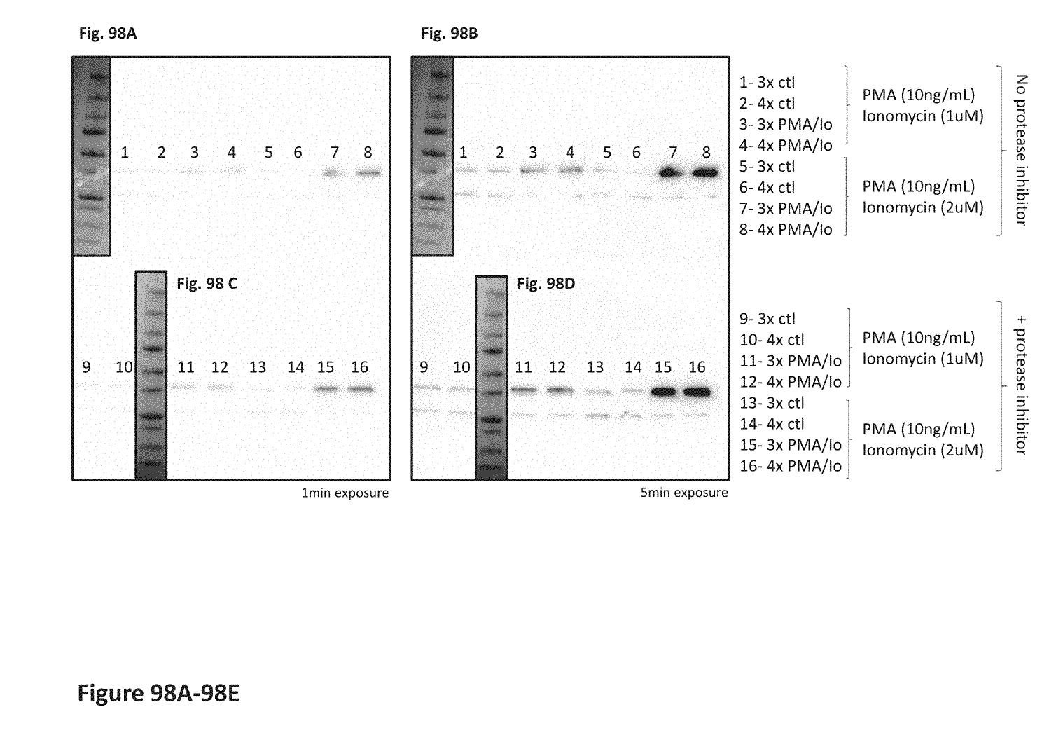

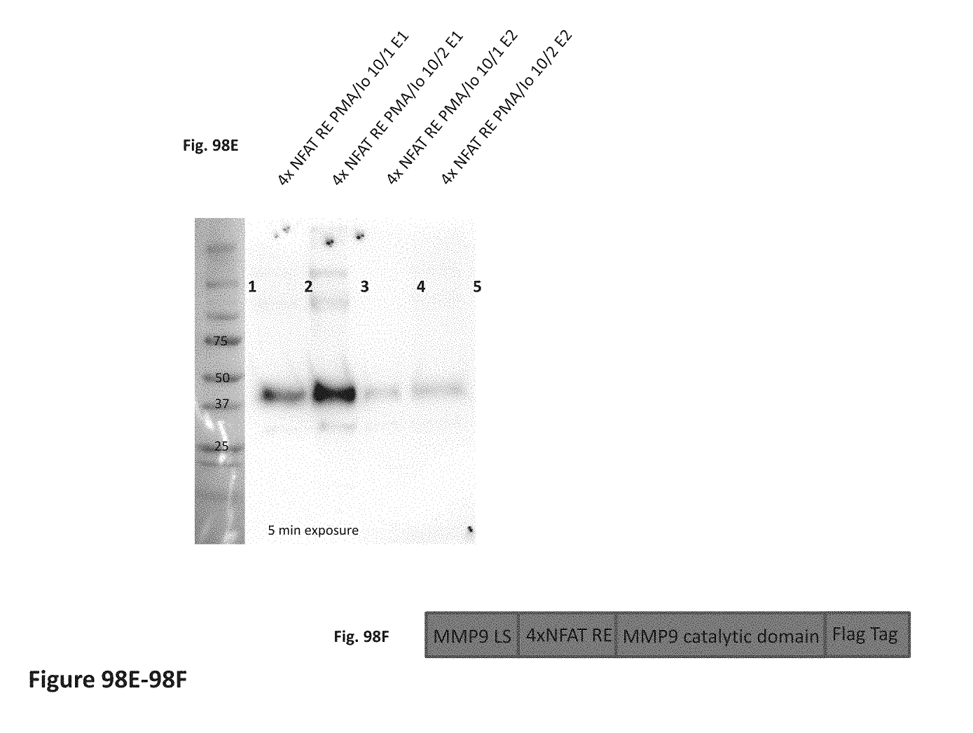

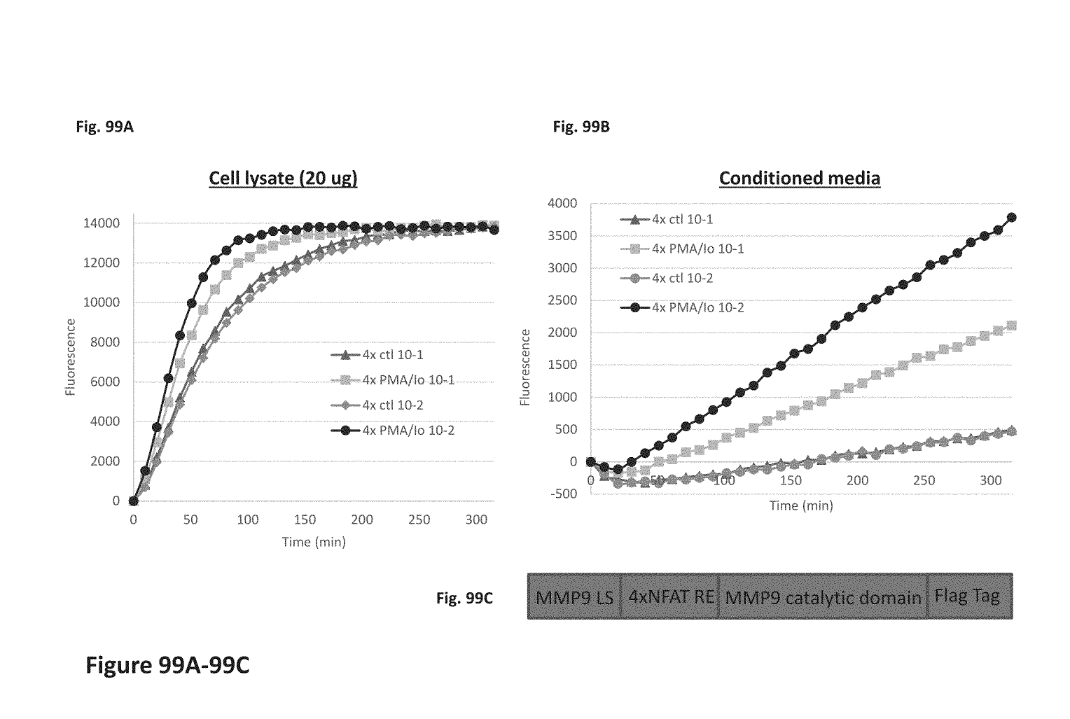

A plasmid was constructed then transfected into HEK293T cells, wherein the gene for MMP9 catalytic domain was inserted downstream of either 3 or 4 NFAT response elements. The NFAT pathway was activated by the addition of PMA at 10 ng/mL and lonomycin at either 1 uM or 2 uM. Lysate from cells transfected with the plasmid containing 3 or 4 repeats of a NFAT Response element, or the conditioned media from the cells, were assayed for the presence of MMP9 in a Western blot assay. As can be seen in FIGS. 98A-98E only the cells that contained the NFAT response elements upstream of the MMP9 and also wherein the NFAT pathway was activated by PMA/Ionomycin, was MMP9 detectable from the lysates and conditioned media. In addition, the amount of MMP9 expressed or secreted was proportional to the concentration of the NFAT pathway activators. We next tested the MMP9 from the lysate and also the MMP9 that was secreted into the conditioned media to see if it was active and able to cleave the MMP9 substrate. FIGS. 99A-99B show graphs of a fluorogenic peptide substrate of MMP9 being cleaved by the cell lysate or conditioned media of HEK293T cells that were transfected with a plasmid containing an MMP9 gene downstream from 4 repeats of an NFAT response element. The MMP9 peptide substrate assay shows that activation of the NFAT pathway by PMA/ionomycin caused an MMP9 to be expressed and secreted and that it was active as evidenced by its ability to cleave a peptide substrate.

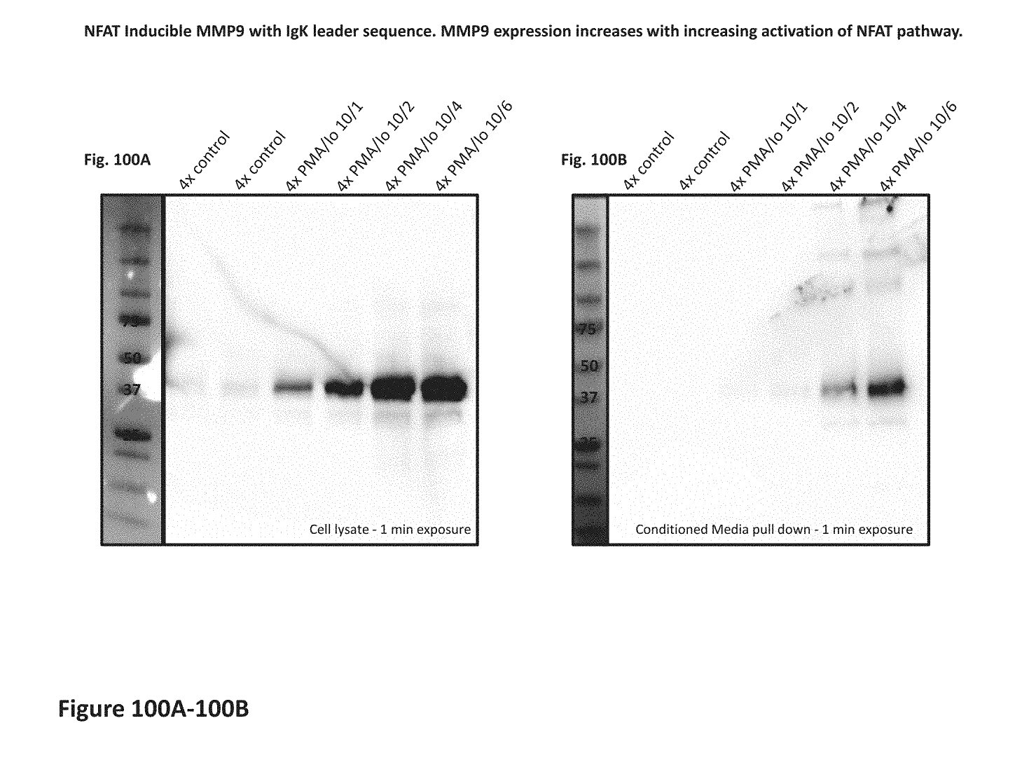

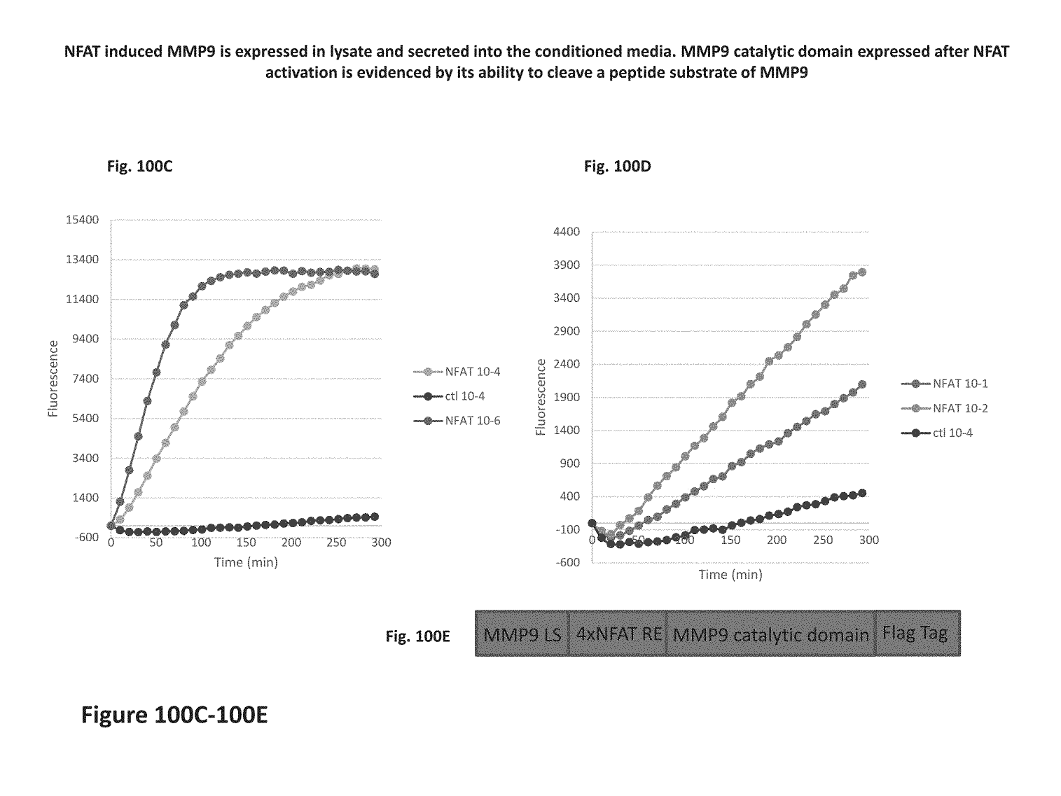

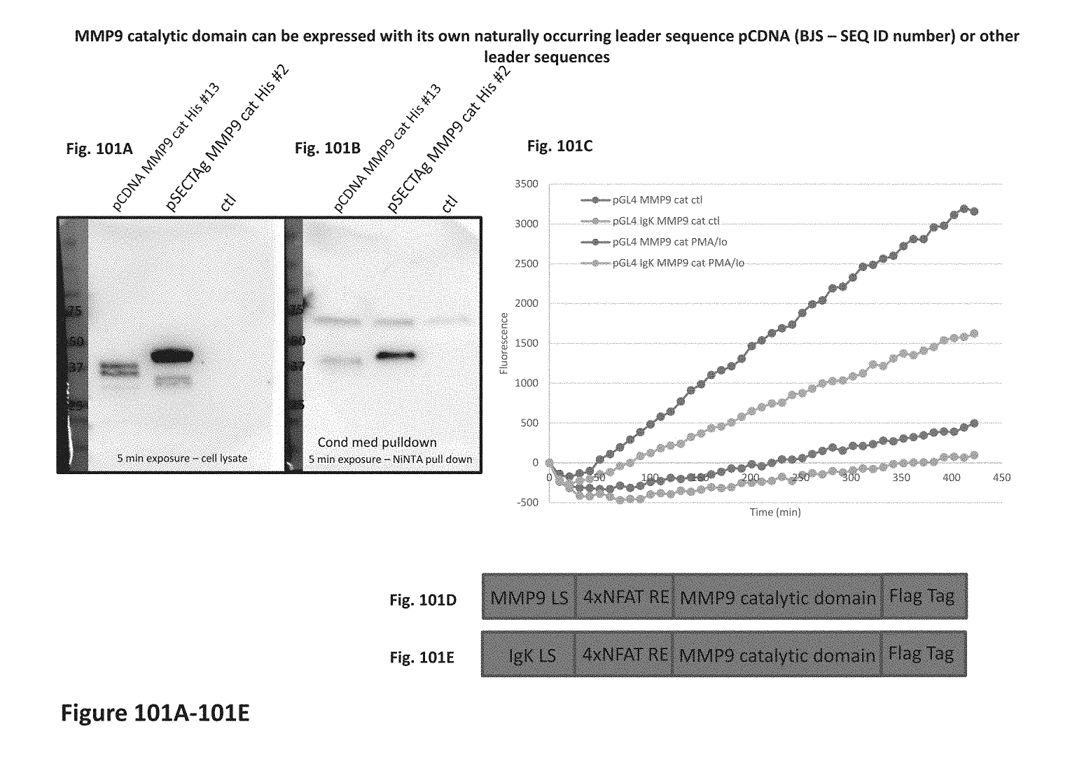

We also tested whether the native leader sequence that is in front of the MMP9 gene is essential or if it could be replaced by other leader sequences that might increase its expression or secretion from the cells. These next experiments showed that the native MMP9 leader sequence can be replaced with other leader sequences. FIGS. 100A-100D show NFAT-induced MMP9 catalytic domain expressed in HEK293T cells wherein the native leader sequence of MMP9 has been replaced by an IgK leader sequence and MMP9 catalytic domain is downstream of 4 repeats of an NFAT response element. FIG. 100A shows photograph of Western blot detecting expression of MMP9 in the cell lysate after activation of the NFAT pathway. FIG. 100B shows photograph of Western blot detecting expression of MMP9 in the conditioned media after activation of the NFAT pathway. FIG. 100C shows graph of MMP9 fluorogenic peptide substrate cleavage by MMP9 catalytic domain expressed and secreted in conditioned media of HEK293T cells wherein the native leader sequence of MMP9 has been replaced by an IgK leader sequence and MMP9 catalytic domain is downstream of 4 repeats of an NFAT response element. FIG. 100D shows graph of MMP9 fluorogenic peptide substrate cleavage by MMP9 catalytic domain expressed and secreted in conditioned media of HEK293T cells wherein the native leader sequence of MMP9 has been replaced by an IgK leader sequence and MMP9 catalytic domain is downstream of 4 repeats of an NFAT response element. FIGS. 101A-101C show MMP9 can be expressed with different leader sequences and also show subsequent activity of each. FIG. 101A shows a Western blot detecting an MMP9 protein in cell lysate wherein the leader sequence upstream of the MMP9 gene is either its native sequence or an IgK sequence. FIG. 101B shows a Western blot detecting MMP9 in conditioned media wherein the leader sequence upstream of the MMP9 gene is its native sequence or an IgK sequence. FIG. 101C shows a graph of an MMP9 peptide substrate cleaved by the expressed MMP9.

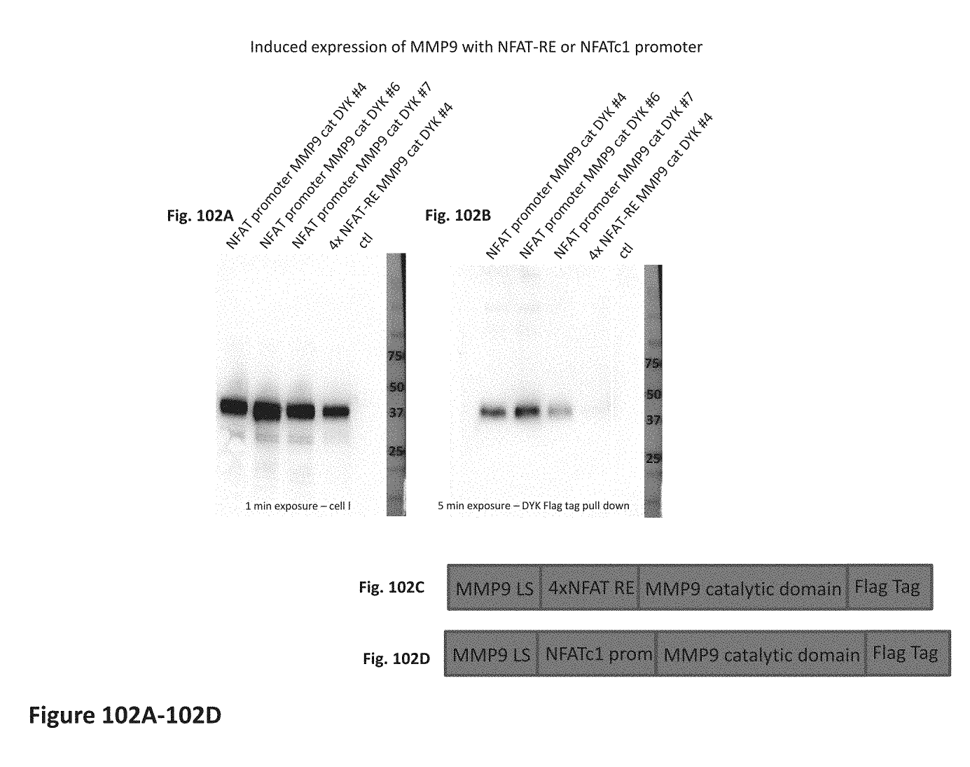

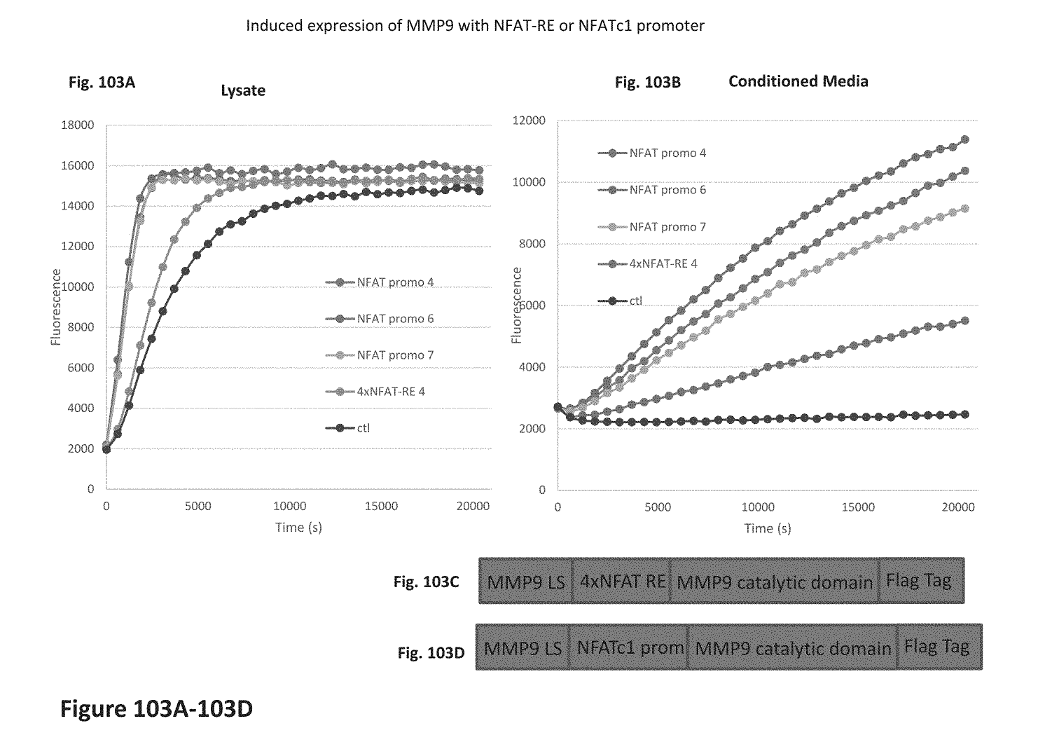

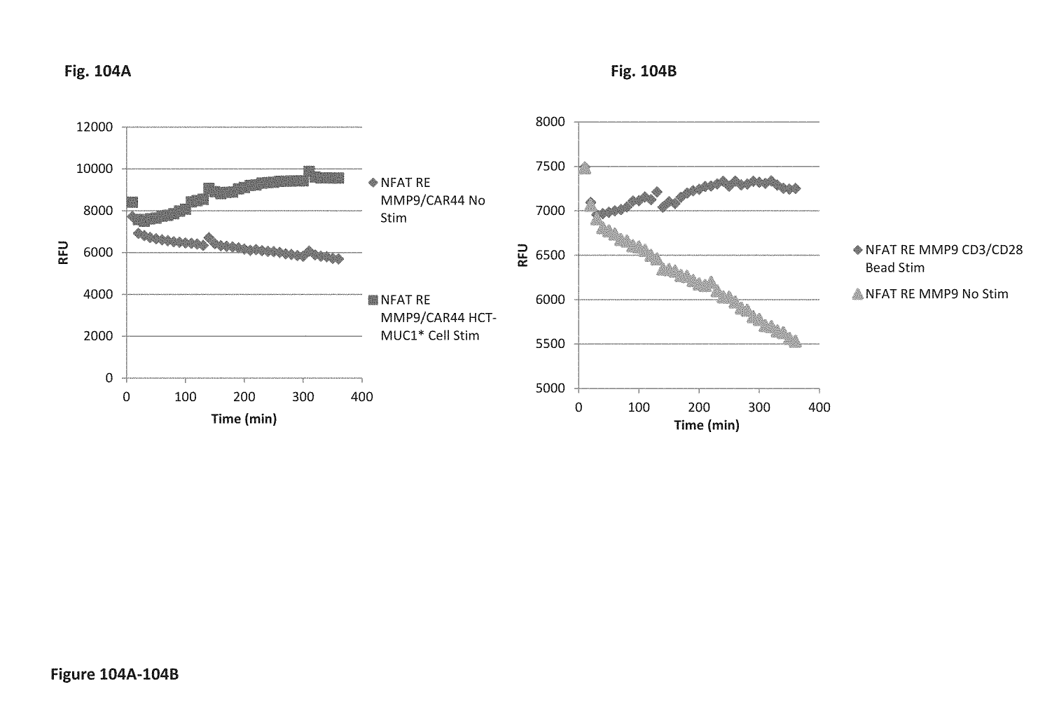

To design a construct that will have cleavage enzyme expression induced by proteins that are expressed or are translocated to the nucleus only after T cell activation, it is possible to have the enzyme gene downstream of response elements or downstream of the promoter of that cleavage enzyme. Another plasmid was made in which the gene for an MMP9 catalytic domain was inserted downstream from a portion of the promoter of NFATc1. The experiments shown in FIGS. 102A-102B compare expression levels of MMP9 expressed off of the NFATc1 promoter or off of 4 repeats of an NFAT response element. They show that both approaches work well. FIGS. 102A-102B show 3 clones 4, 6 and 7 of cells transfected with a plasmid that produces an NFAT inducible MMP9 wherein the NFATc1 promoter sequence is upstream of the MMP9 gene, which in this case is a truncated MMP9 comprising its catalytic domain. Also shown for comparison is a cell transfected with a plasmid that produces an NFAT inducible MMP9 wherein 4 repeats of an NFAT response element sequence are upstream of an MMP9 gene. FIG. 102A shows a Western blot detecting an MMP9 protein in cell lysate. FIG. 102B shows a Western blot detecting MMP9 in the conditioned media. FIGS. 103A-103B show that the MMP9 in the cleared lysate and the MMP9 in the conditioned media are also active as they cleave the MMP9 substrate in the peptide fluorogenic assay.

We next tested whether or not the NFAT-inducible MMP9 would work in human T cells and if it would specifically be expressed and secreted after T cell activation. To test this, the construct having 4 repeats of the NFAT response element were incorporated into a lenti viral vector. Human T cells were transduced with either an NFAT-inducible MMP9 alone, a CAR44 alone or both CAR44 and an NFAT-inducible MMP9. In some cases, the transduced T cells were activated by incubating them with beads coated with anti-CD3 and anti-CD28, which are known to activate T cells. In other cases, the transduced T cells were activated by co-culturing them with beads presenting the synthetic MUC1* peptide or by co-culturing with MUC1* positive cancer cells such as HCT-MUC1* cells.

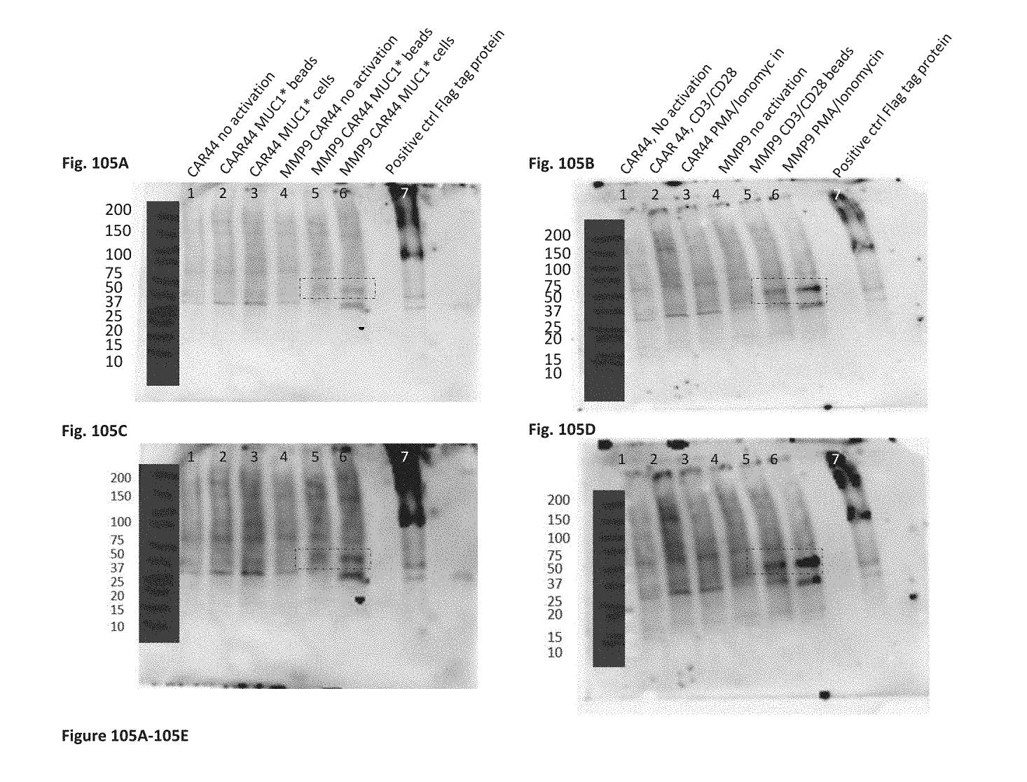

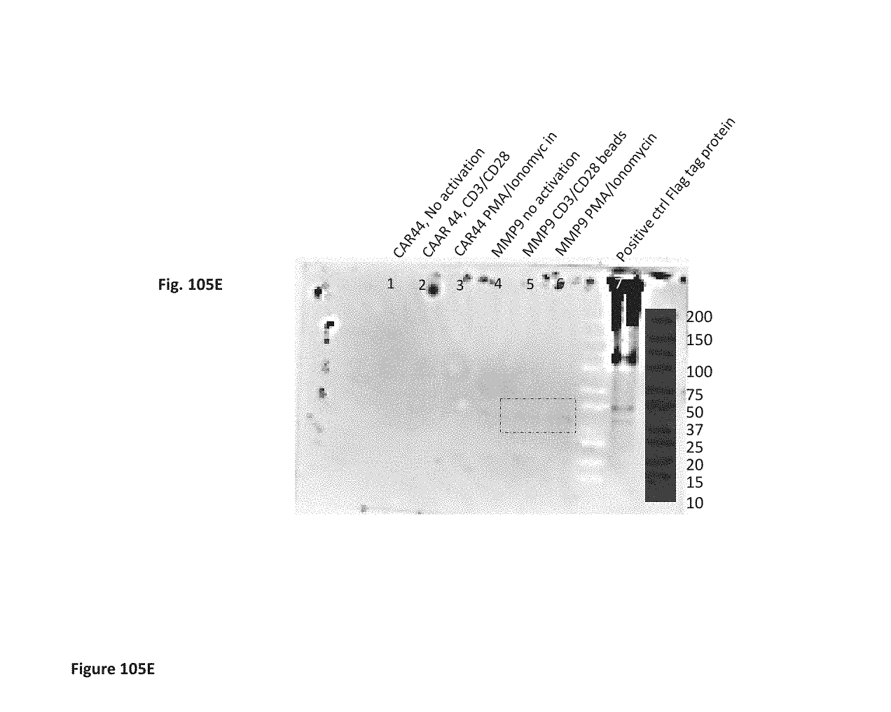

FIGS. 104A-104B show the results of the OMNIMMP9 fluorogenic substrate assay that measures activity of MMP9. Conditioned media from human T cells transduced with NFAT-inducible MMP9 alone or in combination with CAR44 were added to the assay and MMP9 substrate cleavage was measured as a function of time. FIG. 104A shows MMP9 activity when human T cells were transduced with both CAR44 and an NFAT-inducible MMP9 after the cells were activated by co-culturing with HCT-MUC1* cancer cells. The trace that does not show increased substrate cleavage as a function of time is the conditioned media from cells that were not activated. FIG. 104B shows MMP9 activity when human T cells were transduced with just an NFAT-inducible MMP9 after the cells were activated by co-culturing with beads coated with anti-CD3 and anti-CD28 which are known to activate T cells. The trace that does not show increased substrate cleavage as a function of time is the conditioned media from cells that were not activated. FIGS. 105A-105E show photographs of Western blots of human T cells transduced with either CAR44 alone, NFAT-inducible MMP9 alone or transduced with both CAR44 and NFAT-inducible MMP9, wherein the resultant T cells are either not activated, chemically activated by PMA/Ionomycin, activated by co-culturing with beads presenting synthetic MUC1* peptide or co-culturing with MUC1* positive cancer cells. Western blot was probed with an anti-Flag tag also known as DYK tag antibody. Catalytic domain of MMP9 runs with an apparent molecular weight of about 40 kDa. FIGS. 105A-105D show photographs of Western blots of cleared cell lysates. FIG. 105A has Lanes 1-7 loaded with lysates of: Lane 1: T cells transduced with CAR44 and not activated; Lane 2: T cells transduced with CAR44 and activated with beads presenting synthetic MUC1* extra cellular domain peptide; Lane 3: T cells transduced with CAR44 and activated by co-culture with HCT-MUC1* cancer cells; Lane 4: T cells transduced with CAR44 and NFAT-inducible MMP9 but not activated; Lane 5: T cells transduced with CAR44 and NFAT-inducible MMP9 and activated with beads presenting synthetic MUC1* extra cellular domain peptide; Lane 6: T cells transduced with CAR44 and NFAT-inducible MMP9 and activated by co-culture with HCT-MUC1* cancer cells; Lane 7: an irrelevant protein also bearing the Flag DYK tag. Results show that T cells transduced with NFAT-inducible MMP9 only express MMP9 when they are activated by PMA/Ionomycin, MUC1* beads or MUC1* positive cancer cells. T cells transduced with both CAR44 and NFAT-inducible MMP9 only express MMP9 when the T cells are activated by stimulation with MUC1* beads or with MUC1* positive cancer cells. FIG. 105B has Lanes 1-7 loaded with lysates of: Lane 1: T cells transduced with CAR44 and not activated; Lane 2: T cells transduced with CAR44 and activated with beads presenting anti-CD3 and anti-CD28 antibodies that are known to activate T cells; Lane 3: T cells transduced with CAR44 and activated by co-culture with PMA/Ionomycin; Lane 4: T cells transduced with NFAT-inducible MMP9 but not activated; Lane 5: T cells transduced with NFAT-inducible MMP9 and activated with beads presenting anti-CD3 and anti-CD28 antibodies; Lane 6: T cells transduced with NFAT-inducible MMP9 and activated by PMA/Ionomycin; Lane 7: an irrelevant protein also bearing the Flag DYK tag. FIGS. 105C and 105D are darker exposures of the same Western blots shown in FIGS. 105A and 105B, respectively. FIG. 105E is a photograph of a Western blot of cell supernatants of cells transduced as follows: Lane 1: T cells transduced with CAR44 and not activated; Lane 2: T cells transduced with CAR44 and activated with beads presenting anti-CD3 and anti-CD28 antibodies that are known to activate T cells; Lane 3: T cells transduced with CAR44 and activated by co-culture with PMA/Ionomycin; Lane 4: T cells transduced with NFAT-inducible MMP9 but not activated; Lane 5: T cells transduced with NFAT-inducible MMP9 and activated with beads presenting anti-CD3 and anti-CD28 antibodies; Lane 6: T cells transduced with NFAT-inducible MMP9 and activated by PMA/Ionomycin; Lane 7: an irrelevant protein also bearing the Flag DYK tag. Results show that T cells transduced with NFAT-inducible MMP9 express MMP9 when they are activated. T cells transduced with both CAR44 and NFAT-inducible MMP9 are specifically activated when they are co-cultured with beads or cells presenting or expressing MUC1* (FIG. 105A Lane 5 and Lane 6).

In one aspect of the invention, a person diagnosed with cancer or at risk of developing cancer is administered a sufficient amount of an immune cell transduced with both a CAR and a cleavage enzyme. In another aspect of the invention, a person diagnosed with cancer or at risk of developing cancer is administered a sufficient amount of an immune cell transduced with both a CAR and a cleavage enzyme, wherein the cleavage enzyme is on an inducible promoter that is activated by proteins that are expressed when the immune cell becomes activated. In another aspect of the invention, a person diagnosed with cancer or at risk of developing cancer is administered a sufficient amount of an immune cell transduced with both a CAR and a cleavage enzyme, wherein the cleavage enzyme is on an inducible promoter that is activated by one or more NFAT. In one case the NFAT is NFATc1. In another aspect, the NFAT is NFATc3. In another aspect, the NFAT is NFATc2. In any of the instances above, the extra cellular domain of the CAR comprises a fragment of an anti-MUC1* antibody. In one aspect, the anti-MUC1* antibody is MNC2scFv or a humanized form of MNC2scFv. In another aspect, the anti-MUC1* antibody is MNE6scFv or a humanized form of MNE6scFv. In any of the instances above, the immune cell can be a T cell, an NK cell, a mast cell, or a dendritic cell.

It is not intended that the present invention be limited to one or two specific methods of having expression of a cleavage enzyme induced by an activated T cell. We have demonstrated specific expression of a cleavage enzyme only upon T cell activation by constructing a plasmid with the cleavage enzyme gene downstream of an NFAT promoter sequence or downstream of one or more repeats of NFAT response elements. In another aspect of the invention, expression of the cleavage enzyme is induced by constructing a plasmid where the cleavage enzyme gene is inserted downstream of an IL-2 promoter sequence or downstream of an IL-2 response element, then inserting the plasmid into an immune cell. In another aspect of the invention, expression of the cleavage enzyme is induced by constructing a plasmid where the cleavage enzyme gene is inserted downstream of a Calcineurin promoter sequence or downstream of a Calcineurin response element, then inserting the plasmid into an immune cell and then administering to a patient for the treatment or prevention of cancers. There are also drug-inducible plasmids that can be used to induce expression of the cleavage enzyme or used to stop expression induced by an element of an activated T cell. These drug inducible systems may include tetracycline-inducible systems, Tet-on, Tet-off, tetracycline response elements, doxycycline, tamoxifen inducible systems, ecdysone inducible systems and the like.

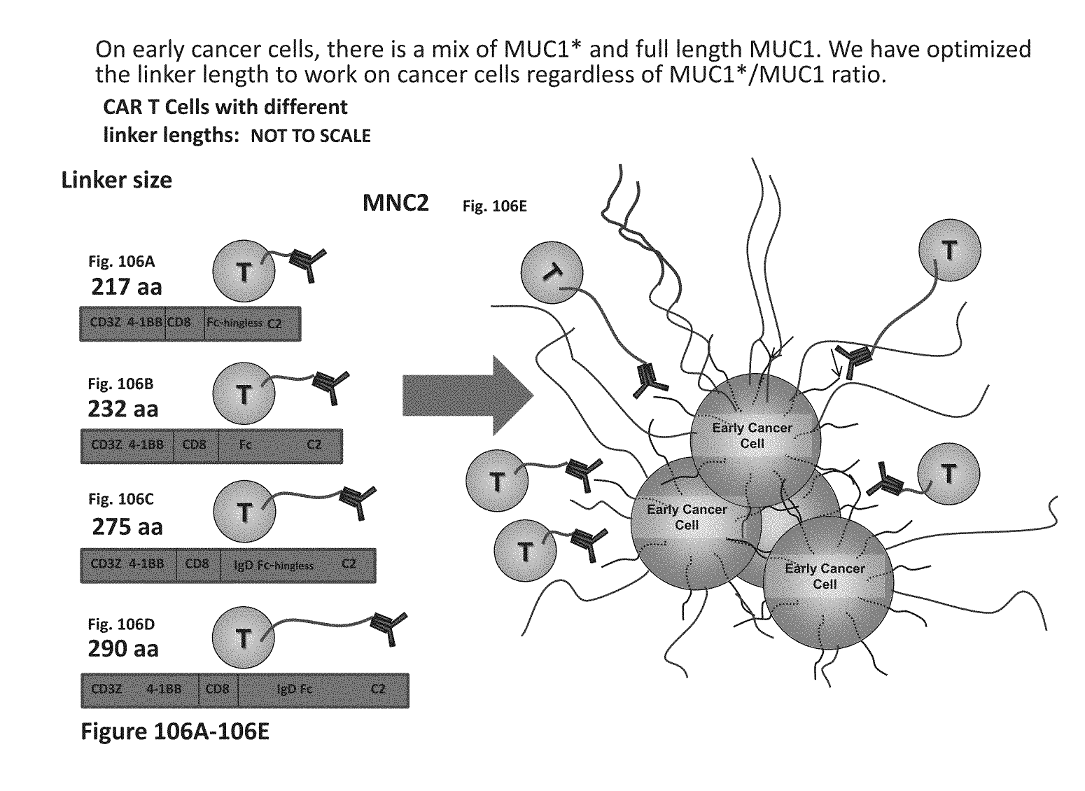

It is not intended that the present invention be limited to one or two specific promoters used in the plasmids encoding the CARs or inducible cleavage enzymes. As is known by those skilled in the art, many promoters can be interchanged including SV40, PGK1, Ubc, CAG, TRE, UAS, Ac5, polyhedron, CaMKIIa, GAL1, GAL10, TEF1, GDS, ADH1, CaMV35S, Ubi, H1 and U6. Another solution to the problem of steric hindrance of CAR T cell access, caused by bulky cell surface proteins such as MUC1-FL, is to increase the length of the linker region of the CAR that is expressed by the T cell. In standard design CARs, the length of the extracellular linker region between the transmembrane portion and the antibody fragment is about 45-50 amino acids in length. We made long-arm CARs where the length of the extracellular linker is extended from about 50 amino acids to 217-290 amino acids. Co-culture assays show that CARs with longer extracellular linkers have improved access to the tumor-associated antigen on the target cancer cells. A cartoon of this strategy is shown in FIGS. 106A-106E.

Published reports of CARs generally use a linker between the transmembrane domain and the antibody fragment, scFv, that is 45-50 amino acids in length and is often the sequence of the extracellular domain of CD8. CAR 44 is an anti-MUC1* CAR whose linker is derived from CD8 extracellular domain and is 45 amino acids in length. To demonstrate that long-arm CARs enable the T cell greater access to tumor associated antigens near the cell surface, we made a series of CARs wherein the anti-MUC1* antibody fragment was MNC2 scFv (SEQ ID NO:655) which was connected to the transmembrane domain via a panel of linkers of variable length and flexibility, wherein the transmembrane domain was that of CD8 (SEQ ID NO:657), followed by co-stimulatory domain 4-1BB (SEQ ID NO:659) then CD3-zeta (SEQ ID NO:661). A panel of linkers were incorporated into this model CAR. An IgG Fc domain which is 232 amino acids in length (SEQ ID NO:663) was used as a linker for an MNC2 CAR (SEQ ID NO:665). An IgD Fc domain which is 290 amino acids in length (SEQ ID NO:667) was used as a linker for an MNC2 CAR (SEQ ID NO:669). An IgG1 hingeless Fc domain linker which is 217 amino acids in length (SEQ ID NO:671) was used as a linker for an MNC2 CAR (SEQ ID NO:673). An IgD hingeless Fc domain linker which is 275 amino acids in length (SEQ ID NO:675) was used as a linker for an MNC2 CAR (SEQ ID NO:677). An IgD linker which is 58 amino acids in length (SEQ ID NO:679) was used as a linker for an MNC2 CAR (SEQ ID NO:681). An X4 linker which is 43 amino acids in length (SEQ ID NO:683) was used as a linker for an MNC2 CAR (SEQ ID NO:685).

These CARs with variable length linkers between the scFv and the transmembrane domain are: CAR15: huE6-IgD-CD8-41BB-3z (SEQ ID NO: 324); CAR16: muE6-IgD-CD8-41BB-3z (SEQ ID NO: 823); CAR17: muC2IgD-CD8-41BB-3z (SEQ ID NO: 825); CAR18: huE6-Fc-CD8-41BB-3z (SEQ ID NO: 311); CAR19: huE6-FcH-CD8-41BB-3z (SEQ ID NO: 316); CAR20: huE6-X4-CD8-41BB-3z (SEQ ID NO: 330); CAR33: huE6-IgD-CD441BB-3z (SEQ ID NO: 327); CAR34: huE6-Fc-CD441BB-3z (SEQ ID NO: 319); CAR35: huE6-FcH-CD441BB-3z (SEQ ID NO: 321); CAR36: huE6-X4-CD441BB-3z (SEQ ID NO: 334); CAR39: muE6-CD28-CD28-CD28-3z (SEQ ID NO: 827); CAR40: muC2-CD28-CD28-CD28-3z (SEQ ID NO: 829); CAR53: huC2-Fc-CD8-41BB-3z (SEQ ID NO: 665 and 733); CAR54: huC2-IgD+Fc-CD8-41BB-3z (SEQ ID NO: 669 and 735); CAR55: huC2-FcH-CD8-41BB-3z (SEQ ID NO: 673 and 737); CAR56: huC2-IgD+FcH-CD8-41BB-3z (SEQ ID NO: 677 and 739); CAR57: huC2-IgD-CD8-41BB-3z (SEQ ID NO: 681 and 741); CAR58: huC2-X4-CD8-41BB-3z (SEQ ID NO: 685 and 743); CAR63: huE6-IgD+Fc-CD8-41BB-3z (SEQ ID NO: 771); CAR64: huE6-IgD+FcH-CD8-41BB-3z (SEQ ID NO: 773); CAR42: hu co-CD19-IgD-CD8-41BB-3z (SEQ ID NO: 831). Additional details regarding these long linker CARs are shown in Table 1. Table 2 shows experimental activity of some of the CARs when transduced into human T cells and co-cultured with cancer cells.

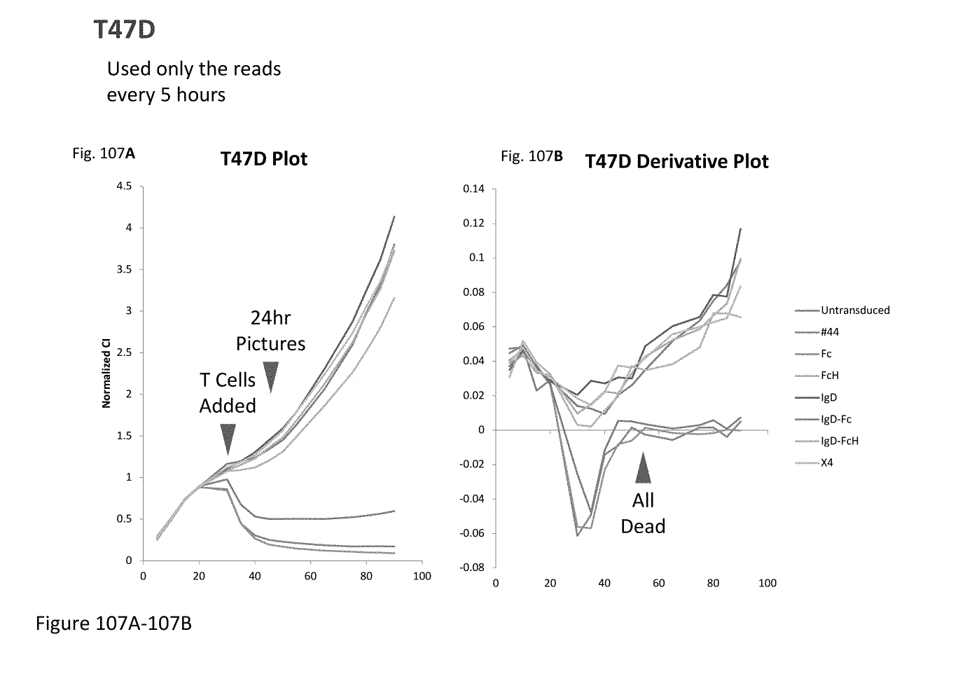

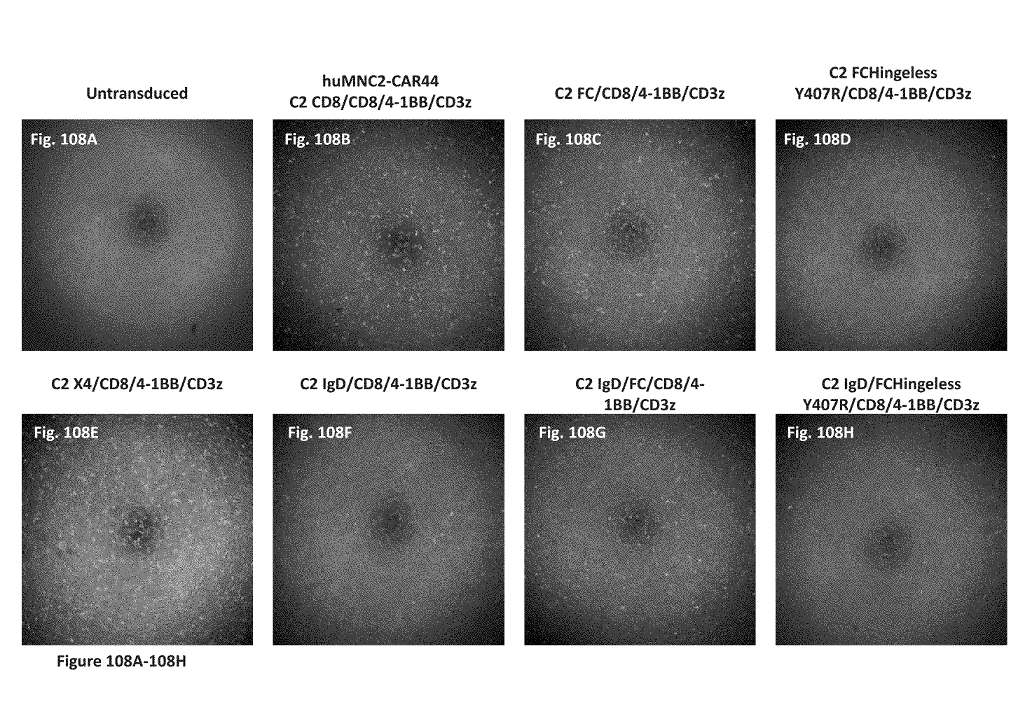

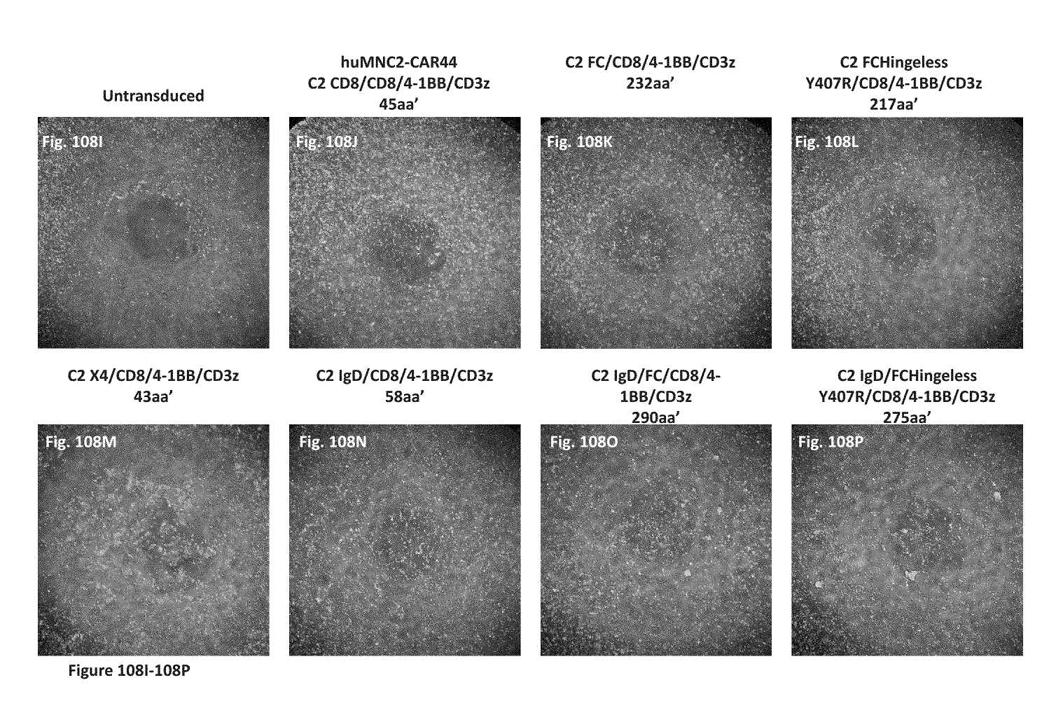

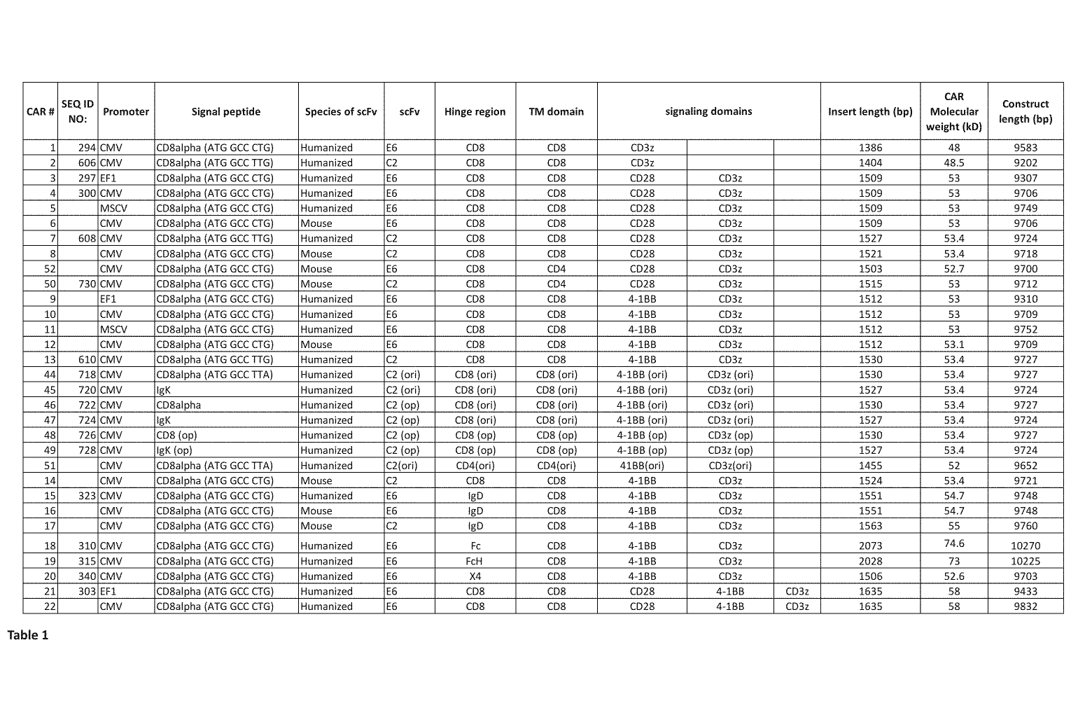

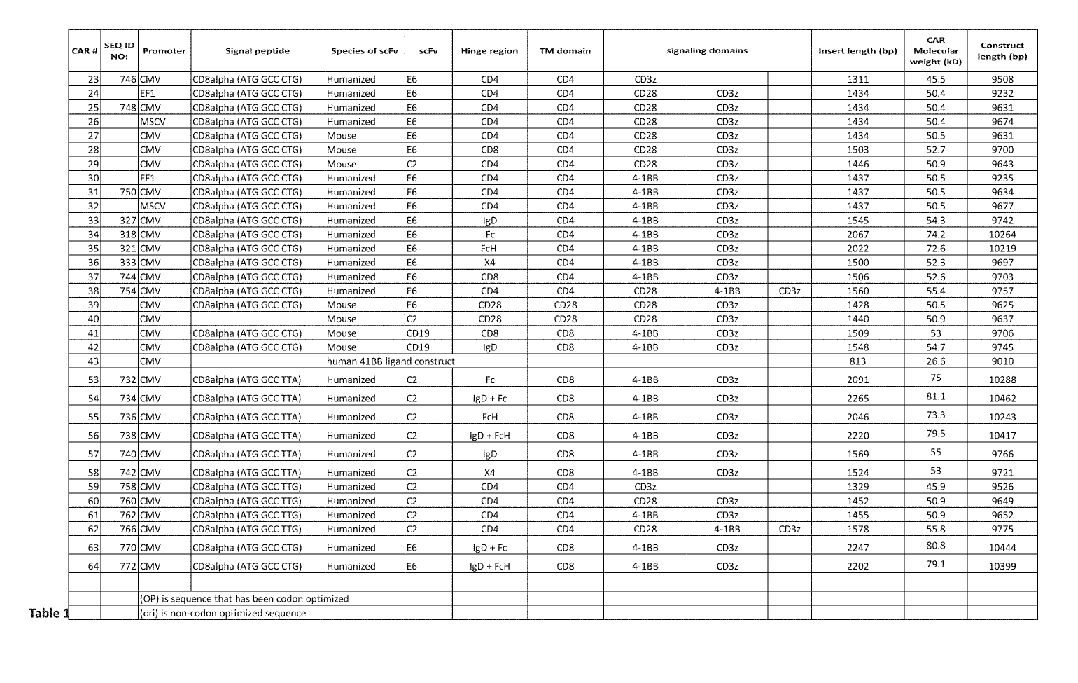

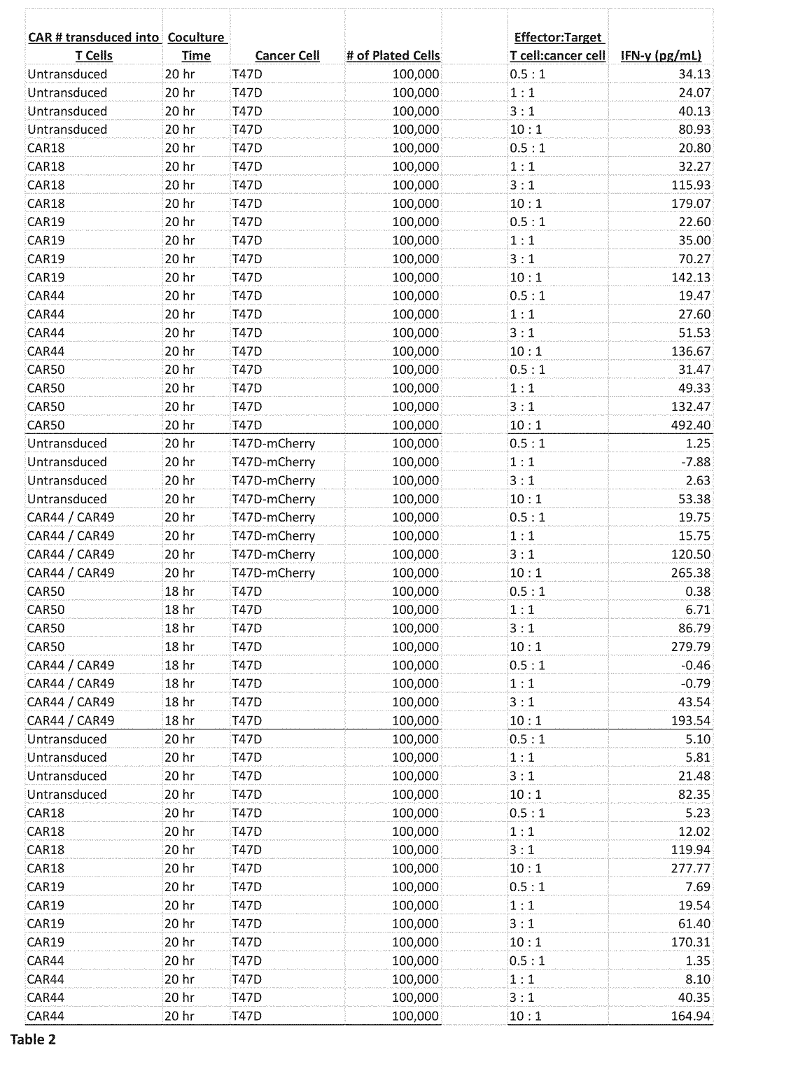

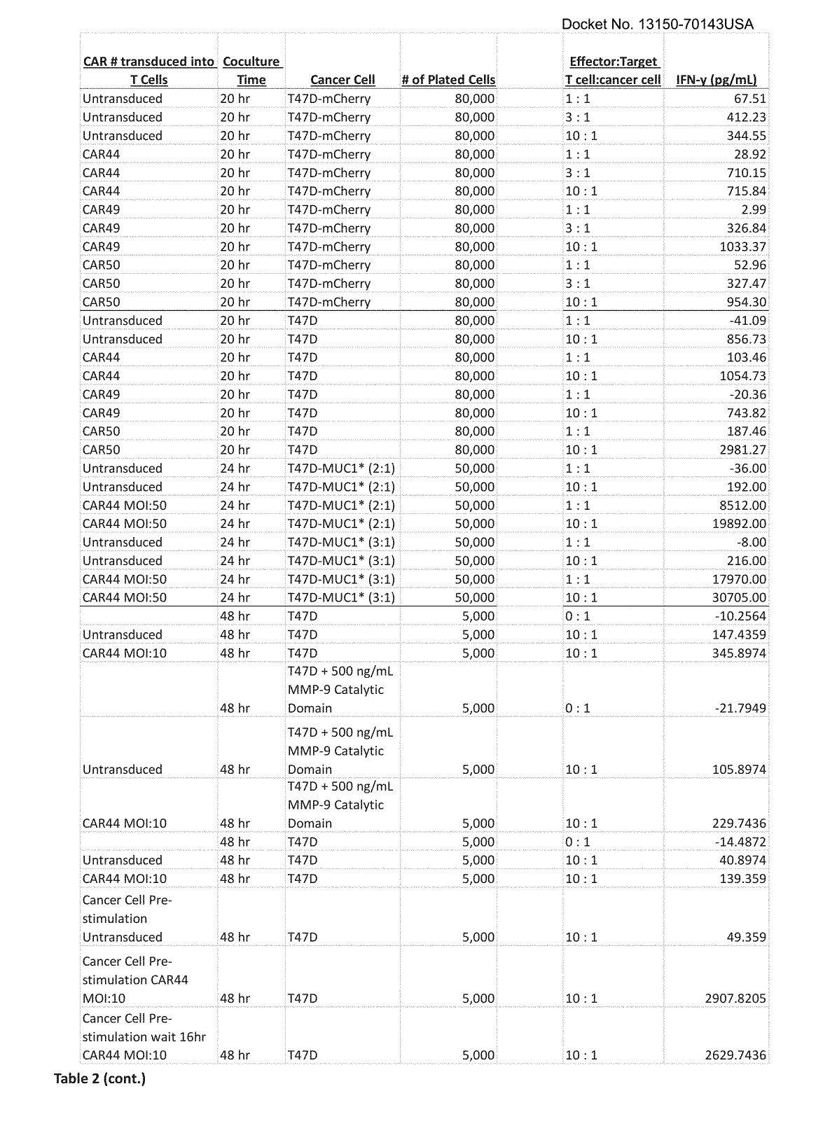

In co-culture experiments, anti-MUC1* CARs with extracellular domain linkers of varying lengths were tested for their ability to specifically kill target MUC1/MUC1* positive cancer cells. xCELLigence scans shown in FIGS. 107A-107B show the results of one experiment. In this experiment the long linker CARs were transduced into human T cells then co-cultured with T47D breast cancer cells. However, some of the CARs that appear not to effectively kill the target cancer cells may just not have been efficiently expressed. Another experiment was performed in order to separate CAR expression from CAR efficacy. HEK293 adherent cells were transduced with a panel of CARs each having different length linkers. The CAR plasmid also carried a GFP marker so expression of each CAR could be measured by the amount of cells that were green. To these cells were added K562 suspension cells that had been stably transfected with MUC1*. K562-MUC1* cells were stained with a red dye, CMTMR. After wash steps, the amount of cells that were yellow (green plus red) indicates the ability of each of the CARs to recognize the target tumor antigen on a cancer cell. As can be seen in FIGS. 108A-108H, the expression levels of the CARs varies greatly. However, expression levels are easily optimized so does not constitute a problem. In looking at FIGS. 1081-108P the number of cells that appear yellow, versus those that remain green, gives more information about which CAR linkers are best to overcome steric hindrance of other surface molecules on the target cancer cells. A significant amount of target cancer cells bound to CAR-expressing cells for CARs having linkers derived from CD8, IgG1 FcH (hingeless), IgD and IgDFcH (hingeless). In addition to length, the linkers tested in these CARs are expected to vary in their rigidity.

Table 2 shows cytokine release data for human T cells transfected with some of the long linker CARs.

We note that “long-arm” CARs that have increased efficacy against solid tumor cancers can be guided by any antibody fragment that recognizes a tumor associated antigen, including MNE6 scFv, MNC2-scFv and other anti-MUC1* antibody fragments. Similarly, the transmembrane portion of the long-arm CARs can be derived from CD8, CD4 or other transmembrane domain. The intracellular tail of the CAR can be comprised of CD3-zeta and any other co-stimulatory domains or combinations thereof including CD28, 4-1BB, and OX40.

In another aspect, the invention is directed to a composition that includes at least two different plasmids transfected into the same immune cell, wherein the first encodes a CAR comprising an antibody fragment, scFv, or peptide that binds to a tumor antigen and the other encodes a gene that is not a CAR, wherein the gene that is not a CAR is expressed from an inducible promoter that is activated by elements of an activated immune cell. In one aspect, the immune cell is a T cell or an NK cell. In one aspect the CAR comprises an antibody fragment, scFv or peptide that binds to the extra cellular domain of MUC1*. In one aspect the CAR comprises an scFv derived from MNC2, MNE6, MNC3 or MNC8. In one aspect the non-CAR species is a cleavage enzyme. In one aspect the cleavage enzyme is MMP2, MMP3, MMP9, MMP13, MMP14, MMP16, ADAM10, ADAM17, ADAM28 or catalytically active fragments thereof. In another aspect the non-CAR species is a cytokine. In one aspect, the Cytokine is IL-7. In one aspect the cytokine is IL-15. In another aspect the cytokine is IL-7 and IL-15. In one case expression of the non-CAR species is induced by elements of an activated immune cell. In one aspect the element of an activated immune cell is an NFAT. In one aspect the NFAT is NFATc1, NFATc3 or NFATc2. Cytokines IL-7 and IL-15 are known to promote T cell persistence. In one aspect of the invention an immune cell described above is administered to a patient for the treatment or prevention of cancer. In one aspect of the invention, the cancer is a MUC1 positive cancer or a MUC1* positive cancer.

In another aspect, the invention is directed to a composition that includes at least two different plasmids transfected into the same immune cell, wherein the first encodes a CAR comprising an antibody fragment, scFv or peptide that binds to the extra cellular domain of an antigen on the surface of a B cell and the other encodes a gene that is not a CAR, wherein the gene that is not a CAR is expressed from an inducible promoter that is activated by elements of an activated immune cell. In one aspect, the immune cell is a T cell or an NK cell. In one aspect the CAR comprises an antibody fragment, scFv or peptide that binds to CD19. In one aspect the CAR comprises sequences derived from SEQ ID NO:830-831. In another aspect the antibody fragment, scFv or peptide binds to a surface antigen of a B cell or a B cell prescursor, or binds to CD19, CD20, CD22, BCMA, CD30, CD138, CD123, CD33 or LeY antigen. In one aspect the non-CAR species is a cleavage enzyme. In another aspect the non-CAR species is a cytokine. In one aspect, the Cytokine is IL-7. In one aspect the cytokine is IL-15. In another aspect the cytokine is IL-7 and IL-15. In one case expression of the non-CAR species is induced by elements of an activated immune cell. In one aspect the element of an activated immune cell is an NFAT. In one aspect the NFAT is NFATc1, NFATc3 or NFATc2. that is not a CAR, wherein the gene that is not a CAR is expressed from an inducible promoter wherein expression is induced by elements of an activated immune cell. In one aspect the immune cell transfected or transduced with the composition is administered to a patient for the treatment or prevention of cancer. In one case the cancer is a leukemia, lymphoma or blood cancer.

It is not intended for the invention to be limited by a specific method or technology for inserting the gene or plasmid comprising a sequence encoding a CAR or activated T cell inducible protein or peptide there encoded. For example, the gene encoding the CARs and activated T cell induced genes described herein can be virally transduced into an immune cell using viruses, which may or may not result in the CAR gene being integrated into the genome of the recipient cell. Virus delivery systems and viral vectors include but are not limited to retroviruses, including gamma-retroviruses, lentivirus, adenoviruses, adeno-associated viruses, baculoviruses, poxvirus, herpes simplex viruses, oncolytic viruses, HF10, T-Vec and the like. In addition to viral transduction, CARs and activated T cell induced genes described herein can be directly spliced into the genome of the recipient cell using methods such as CRISPR technology, CRISPR-Cas9 and -CPF1, TALEN, Sleeping Beauty transposon system, and SB 100×.

Bulky cell surface proteins such as MUC1-FL can also cause a steric hindrance problem for BiTEs. A BiTE is a two-headed bi-specific antibody wherein one head binds to a T cell and the other head binds to a tumor-associated antigen. In this way, the BiTE links together the T cell and the tumor cells. The antibody that binds to the T cell should be an antibody that activates the T cell, such as an antibody against CD3 such as OKT3 scFv (SEQ ID NO:687) or CD28. To solve the steric hindrance problem, the linker between the T cell specific antibody and the tumor specific antibody is lengthened. Examples of BiTEs with extended linkers Anti-CD3-linker-anti-MUC1*, are shown as SEQ ID NOS:689, 691, 693, 695, 697, and 699.

In another aspect of the invention, an anti-MUC1* single chain molecule is fused to a cleavage enzyme or a catalytically active fragment of a cleavage enzyme. In one aspect of the invention, the cleavage enzyme is MMP9 (SEQ ID NO:701). In another aspect of the invention, the enzyme is a catalytically active fragment of MMP9 (SEQ ID NO:703). In some cases, the antibody fragment of the CAR is chosen for its ability to recognize MUC1* when cleaved by that specific cleavage enzyme. In one embodiment, the cleavage enzyme is MMP9, MMP3, MMP14, MMP2, ADAM17, ADAM TS16, and/or ADAM28. In one embodiment, the antibody or antibody fragment binds to a peptide having the sequence of SEQ ID NO:2 (PSMGFR) GTINVHDVETQFNQYKTEAASRYNLTISDVSVSDVPFPFSAQSGA, PSMGFR N-10, QFNQYKTEAASRYNLTISDVSVSDVPFPFSAQSGA, or PSMGFR N+18 SNIKFRPGSVVVQLTLAFREGTINVHDVETQFNQYKTEAASRYNLTISDVSVSDVPFPFSAQS GA. “PSMGFR N+18” refers to a fragment of MUC1 receptor in which 18 amino acid residues have been added at the N-terminal end of PSMGFR segment within the MUC1 receptor of SEQ ID NO: 1. In another embodiment, cleavage enzymes MMP9 and MMP3 are transduced into a T cell that is also transduced with a CAR with an antibody fragment that is a fragment of MNC2.

Methods Used in Carrying Out Experimentation in Relation to the Present Invention

1. Lentivirus Production and Viral Transduction of Immune Cells

HEK293 or HEK293T cells (ATCC) were used to produce lentivirus. The day prior transfection plates (6 well plate) were coated with poly-D-lysine and cells seeded so that cell density reaches 90-95% at the time of transfection and cultures in a 5% CO2 atmosphere. The next day cells were transfected with Lipofectamine 3000 (life technologies) and Opti-MEM® I Reduced Serum Medium according to the manufacturer instructions (0.75 ug of lentiviral expression vector and 2.25 ug of pPACKH1 packaging mix was used). After 6 h incubation, the media was changed and media containing lentivirus was harvested after 24 and 48 hours. Lentivirus was concentrated with Lenti-X concentrator (Clontech) and titer was calculated using the Lenti-X p@4 Rapid Titer Kit (Clontech). Lentivirus was store at −80 C in single-use aliquots.

Transduction of Immune Cells with Constructs Including CARs

Human T cells, if frozen, were thawed and pre-warmed in 100-200 units IL-2 and TexMACS medium, 20 ml, and pelleted by centrifugation. Cells were resuspended in 10 ml of medium and cultured at 37° C., 5% C02 at 1×10⁶ cells/ml in complete medium with anti-CD3/anti-CD28 beads (TransAct kit).

After 4 days in culture, cells were counted and 450 ul of cell suspension was placed in single well of a 24-well plate at a density of approximately 1×10⁶ cells/ml. Cells were allowed to settle. 150 ul was carefully removed from the top of each well. To each well was added an appropriate dilution of lentiviral vector, diluted in plain TexMACS medium, along with protamine sulfate to a final concentration of 10 ug/ml, in a 150 ul volume, for a final total volume of 450 ul per well and incubated for 24 hrs. Transduced cells were removed, pelleted by centrifugation, and resuspended in fresh medium, adjusting cell density, not to exceed 1.0×10⁶ cells/ml. Transduced T cells can be expanded and frozen or used directly. Typically transduced T cells are used or frozen between Day 7 and Day 20 post activation with IL-2 and TransAct media.

2. Comparing CAR T Cell Activity Among Several Anti-MUC1* CARs

Human T cells (ALLCELLS) were transduced with anti-MUC1* CAR18, CAR19, CAR44, CAR49, CAR44 and CAR49 or CAR50. The CAR constructs all had a GFP marker so that CAR T cells are green and untransduced T cells (FIG. 80A) are clear. CAR18 is huMNE6scFv-Fc-CD8-41BB-3z. CAR19 is the same except instead of a portion of an Fc region for the linker between scFv and transmembrane region, CAR19 has an Fc region with the hinge portion mutated out. CAR44 is huMNC2-scFv-CD8-CD8 (transmembrane-41BB-3z). CAR49 is the same as CAR44 except that CAR44 has a CD8 leader sequence and CAR49 has an IgK leader sequence. CAR50 is the same as CAR44 except that CAR50 has a murine MNC2-scFv and a CD4 transmembrane domain. Table 1 gives details of each CAR construct. The CAR T cells were then incubated for 18 hours with HCT-MUC1* cancer cells that had been stably transfected with mCherry (red). When T cells recognize a target cell, they cluster the target cells and begin to kill them. As can be seen in FIGS. 80A-80F the green CAR T cells are effectively clustering and killing the target MUC1* positive cancer cells.

3. Confocal Imaging of CAR T Cells Giving the “Kiss of Death” to MUC1* Positive Cancer Cells.

Human T cells that were transduced with CAR44 were co-cultured for 24 hours with MUC1* positive cancer cells that were stably transfected with GFP (green). All of the cells were stained with DAPI (blue). Granzyme B was stained with a fluorophore. After T cell activation, they express perforin that is thought to make a hole in the target cancer cells. The T cell then injects the cancer cell with granzyme B (yellow) which then induces apoptotic pathways, resulting in cancer cell lysis. FIGS. 81A-81D show photographs of human huMNC2-CAR44 T cells injecting granzyme B (yellow) into MUC1* positive and GFP positive (green) DU145 prostate cancer cells. FIG. 81A is a 4× magnified photograph. FIG. 81B is a 20× magnified photograph. FIG. 81C is a 20× magnified photograph. FIG. 81D is a 40× magnified photograph.

5. Analysis of CAR T Cell Induced Killing of MUC1* Positive Cancer Cells by FACS Analysis

FIGS. 82A-82B show the killing effect of huMNC2-CAR44 T cells on T47D MUC1* positive breast cancer cells, wherein the breast cancer cells have been transfected with increasing amounts of additional MUC1*. As can be seen, the killing effect of the huMNC2-CAR44 T cells increases as the amount of target MUC1* expressed on the cells increases. FIG. 82A is a graph of target cell killing as measured by FACS. FIG. 82B is a graph of an ELISA assay in which the supernatant from the huMNC2-CAR44 T cells in co-culture with the T47D cells is probed for the presence of secreted interferon gamma, which is a sign of T cell activation.

There are many methods for analyzing cytotoxicity by FACS. In this example, human T cells were isolated from whole blood according to standard protocols. The T cells were then separately transduced twice with lenti virus bearing the CAR constructs, wherein the CAR constructs bear a GFP tag. Following 2-3 days of culture in RPMI 10% FBS and IL-2, the cells were stained with F(ab′)2 to label surface expression of MN-E6, MN-C2, MN-C3 and MN-C8. Cells were then sorted by flow cytometry for Fab-positive, GFP-positive cells. That means that the double positive population had a CAR inserted and that the CAR exposed the correct antibody fragment. The CAR T cells were then ready to be mixed with the MUC1* negative control cells or the target MUC1* positive cancer cells.

The target cells were prepared as follows: Harvest target cells and resuspend cells in serum-free medium containing 15 uM of CMTMr dye (Cell Tracker Orange, 5-and-6-4-chloromethyl benzoyl amino tetramethylrhodamine, Thermo Fisher) at 1-1.5×10⁶ cells/mL. Incubate 30 min under growth conditions appropriate to particular cell type. Wash in culture media and transfer stained cells to a new tube and incubate the cells 60 min in media. Wash 2 more times in culture media to get rid of all excess dye. Set up the assay in 24 well plates with 0.5 ml media total volume. Resuspend the target cells (and control target cells) so that there are always 20,000 cells per well (20,000 cells/250 ul). Plate 250 ul in each well. Add 250 ul of the T cells so that the ratio of T cell: target cells=20:1, 10:1, 5:1 or 1:1. Analyse cells after 24 h and 72 h. For suspension target cells, take off the 0.5 ml media from the well and place in tube, wash the well with 0.5 ml media or PBS. For adherent target cells, take off the 0.5 ml media from the well and place in tube, wash the well with 0.5 ml PBS. Add the PBS to the same tube and add 120 ul trypsin to the well. Incubate for 4 min then add 0.5 ml media to neutralize trypsin and place that in the tube as well. Spin cells and resuspend pellet in 100 ul FACS buffer. Spin cells again. Resuspend cells in 100 ul buffer+5 ul anti-CD3 antibody, for 30 min on ice (to stain T cells). After 30 min, wash stained cells 2× with FACS buffer and resuspend in 250 ul buffer. Run the cells through the filter cap of the FACS tube. 10 min prior to analysis, add 10 ul 7AAD dye to each tube and analyze with Fortessa under the Cytotoxicity template. FIGS. 83A-83D show the results of FACS analysis of huMNC2-CAR44 T cells after 24 hours of co-culture with MUC1* positive cancer cells. FIG. 83A is a graph of FACS data showing the percentage of T47D cancer cells that were killed by huMNC2-CAR44 T cells (blue bars), compared to untransduced T cells (red bars). The X-axis shows the ratio of T cells to cancer cells. FIG. 83B is a graph of FACS data showing the percentage of K562-MUC1* cancer cells that were killed by huMNC2-CAR44 T cells (blue bars), compared to untransduced T cells (red bars). FIG. 83C shows the FACS scans wherein the T47D breast cancer cells were stained with the dye CMTMR. Sytox blue is a dead cell stain. Dead cancer cells are those in quadrants 2 and 3. FIG. 83D shows the FACS scans wherein the K562-MUC1* cancer cells were stained with the dye CMTMR. Sytox blue is a dead cell stain. Dead cancer cells are those in quadrants 2 and 3.

IFN-γ secretion in media was measured using a human IFN-γ ELISA kit (Biolegend). Plates were coated with an anti-IFN-γ antibody (capture antibody, 1× in coating buffer). After overnight incubation at 4° C., the plate was washed 4 times with PBS-T and blocking solution was added to block remaining binding site on the well. After 1 h at RT (shaking at 500 rpm) the plate was washed 4 times with PBS-T and conditioned media (CM) and IFN-γ standard, was added. After 2 h at RT with shaking, the plate was washed 4 times with PBS-T and detection antibody (1×), was added. After 1 h at RT with shaking, the plate was washed 4 times with PBS-T and Avidin-HRP (1×) was added. After 30 min at RT with shaking, the plate was washed 5 times with PBS-T (soak 1 min each wash) and TMB substrate solution was added. The reaction was stopped after 20 min by adding the stop solution and absorbance was read at 450 nm (minus absorbance at 570 nm) within 15 min of stopping.

6. Analysis of CAR T Cell Induced Killing of MUC1* Positive Cancer Cells by xCELLigence

In addition to FACS analysis, many researchers now use an xCELLigence instrument to measure CAR T killing of cancer cells. The xCELLigence instrument uses electrode arrays upon which cancer cells are plated. The adherent cancer cells insulate the electrode and so cause an increase in impedance as they grow. Conversely, T cells are not adherent and remain in suspension so do not contribute to insulation of the electrode which would increase impedance. However, if the T cells or CAR T cells kill the cancer cells on the electrode plate, the cancer cells ball up and float off as they die, which causes the impedance to decrease. The xCELLigence instrument measures impedance as a function of time, which is correlated to cancer cell killing. In addition, the electrode plates also have a viewing window. When CAR T cells effectively kill the adsorbed target cancer cells, there is a decrease in impedance but also one can see that there are no cancer cells left on the plate surface.

In most of the XCELLigence experiments, 5,000 cancer cells were plated per well of a 96-well electrode array plate. Cells were allowed to adhere and grow for 24 hours. CAR T cells were then added at an Effector to Target ratio (E:T) of 0.5:1, 1:1, 2:1, 5:1, 10:1 and sometimes 20:1. The E:T ratio assumes 100% transduction of the CAR into the T cells, when the actual transduction efficiency is 40%.

The xCELLigence instrument records impedance as a function of time and experiments can go on for up to 7 days.

FIG. 78, FIG. 79, FIG. 84H, FIG. 85H, FIGS. 86A-86C, FIGS. 89A-89C, FIGS. 90A-90D, and FIGS. 107A-107B all show results of CAR T and cancer cell experiments performed on an xCELLigence instrument.

7. Anti-MUC1* CAR T Cell Therapy in Mice Bearing Human Tumors

Female NOD/SCID/GAMMA (NSG) mice between 8-12 weeks of age were implanted with 500,000 human cancer cells, wherein the cancer cells had previously been stably transfected with Luciferase. Mice bearing Luciferase positive cells can be injected with the enzyme's substrate Luciferin just prior to imaging, which makes the cancer cells fluoresce. The cancer cells are imaged in live mice within 10-15 minutes after injection with Luciferin on an IVIS instrument. The readout is flux or photons per second. Tumors were allowed to engraft until tumors were clearly visible by IVIS.

FIGS. 91A-91Y show fluorescent photographs of mice taken on an IVIS instrument. NSG (NOD/SCID/GAMMA) immune compromised mice that on Day 0 were subcutaneously implanted on the flank with 500,000 human MUC1* positive cancer cells that had been stably transfected with Luciferase. Tumors were allowed to engraft. On Day 5 after IVIS measurement and on Day 12, animals were injected with 10 million of either human T cells transduced with huMNC2-scFv-CAR44, untransduced T cells or PBS. 5 million T cells were injected intra-tumor and 5 million T cells were injected into the tail vein. 10 minutes prior to IVIS photographs, mice were injected intraperitoneally (IP) with Luciferin, which fluoresces after cleavage by Luciferase, thus making tumor cells fluoresce.

FIGS. 92A-92J show fluorescent photographs of mice taken on an IVIS instrument. NSG (NOD/SCID/GAMMA) immune compromised mice that on Day 0 were subcutaneously injected into the flank with 500K human BT-20 cells which are a MUC1* positive triple negative breast cancer cell line. The cancer cells had been stably transfected with Luciferase. Tumors were allowed to engraft. On Day 6 after IVIS measurement, animals were given a one-time injection of 10 million of either human T cells transduced with huMNC2-scFv-CAR44 or untransduced T cells. 5 million T cells were injected intra-tumor and 5 million were injected into the tail vein. 10 minutes prior to IVIS photographs, mice were IP injected with Luciferin.

FIGS. 93A-93H show fluorescent photographs of mice taken on an IVIS instrument. NSG (NOD/SCID/GAMMA) immune compromised mice that on Day 0 were injected into the intraperitoneal cavity (IP) with 500K human SKOV-3 cells which are a MUC1* positive ovarian cancer cell line. The cancer cells had been stably transfected with Luciferase. Tumors were allowed to engraft. On Day 3 after IVIS measurement, animals were IP injected with 10M either human T cells transduced with huMNC2-scFv-CAR44, untransduced T cells or PBS. Animals were IVIS imaged again on Day 7. 10 minutes prior to IVIS photographs, mice were IP injected with Luciferin.

8. Confocal Analysis of MMP9 Treated Cells

HCT-MUC1-41TR also known as HCT-MUC1-18 cells that stably express MUC1 full length were seeded in 6 channel u-slide VI 0.4 (Ibidi, WI) in DMEM+10% FCS. 48 h later, cells were washed with 120 uL of PBS pH 7.4 and MMP9 catalytic domain (Enzo Life Sciences, NY), diluted in serum free medium (DMEM), was added at different concentrations (40 uL at 0, 12.5, 25, 50 and 100 ng/mL). After 1 h at 37° C. in a CO2 incubator, cells were washed twice with 120 uL of cold PBS pH 7.4 and fixed for 8 min in 4% PFA (30 uL). Cells were washed 3 times with cold PBS pH 7.4 and blocked with a 5% BSA solution in PBS pH 7.4 (40 uL) for 30 min at 4° C. (with shaking). After washing cells with cold PBS pH 7.4 (1×), cells were incubated overnight at 4° C. (with shaking) with 125 ug/mL of MNC2 diluted in PBS pH 7.4 (100 uL). Next day, cells were washed 3× with 120 uL of PBS pH 7.4 and incubated 2 h at 4° C. (with shaking) with goat anti-mouse IgG PE (Biolegend, CA) diluted in PBS pH 7.4 (100 uL, 1:200). After incubation, cells were washed 1× with 120 uL of PBS pH 7.4 and 2× with 120 uL of PBS pH 7.4+2.5 uM Hoechst 33342. Finally, cells were mounted with Ibidi mounting media (Ibidi, WI). Results show that addition of MMP9 induced cleavage of full-length MUC1 to a MUC1* form that was recognized by anti-MUC1* monoclonal antibody MNC2 (FIG. 96A-96E). This shows that MMP9 cleaves MUC1 at a site that is recognized by MNC2.

9. NFAT-Induced MMP9 Catalytic Domain Expression

Vectors containing either 4 repeats of a NFAT response element or the NFATc1 promoter followed by the MMP9 catalytic domain were transiently transfected into HEK293TN cells (System Biosciences, CA) with Lipofectamine 3000 (ThermoFisher Scientific, MA) according the manufacturer manual. After 24-30 h, media was changed to DMEM+1% FBS+10 ng/mL PMA (Cayman Chemical, MI) and lonomycin (1-6 uM, Cayman Chemical, MI). Media and cells were collected after 18 h incubation for analysis.

Expression and secretion of MMP9 was confirmed by Western blot analysis of the cell lysates and conditioned media according to the following protocols. Cells were lysed for 20 min on ice with lysis buffer (50 mM Tris, 150 mM NaCl and 1% Triton X100). For Western blot, 100 ug of protein were separated by gel electrophoresis (4-15% Mini-PROTEAN® TGX™ Precast Protein Gels, BioRad, CA) followed by transfer to PVDF membrane (BioRad, CA). The membrane was briefly rinsed with PBS-T and then blocked for 1 h at room temperature with a solution of 3% non-fat milk (BioRad, CA). For Flag tagged protein, the membrane was quickly washed and incubated with a rabbit anti-DYKDDDDK epitope Tag antibody (Biolegend, CA) was diluted in 1% non-fat milk (1:2000) for 2 h at room temperature. For His tagged protein, the membrane was quickly washed and incubated with a rabbit anti-6× His tag antibody HRP (Abcam, MA) diluted in 1% non-fat milk (1:10000) for 1 h at room temperature. For Flag tagged protein, the membrane was then washed 3 times for 10 min with PBS-T and incubated with goat anti-Rabbit HRP antibody diluted in 1% non-fat milk (1:2500) for 1 h at room temperature. For His tagged protein and after the secondary antibody incubation for the Flag tagged protein, the membrane was processed after being washed 3 times for 10 min with PBS-T using Clarity™ Western ECL Substrate (BioRad, CA).

In some cases, the protein was first immunoprecipitated before analysis. Flag tagged MMP9 catalytic domain was immunoprecipitated from conditioned media (˜2 mL) using an anti-DYKDDDDK Tag (L5) affinity gel (Biolegend, CA) according to manufacturer manual. Pull down proteins were used for Western blot analysis or cleavage assay.

FIGS. 98A-98F are photographs of Western blots of cell lysates probed with an antibody that recognizes the MMP9 construct that was transfected. A plasmid was constructed then transfected into HEK293T cells, wherein the gene for MMP9 catalytic domain was inserted downstream of either 3 or 4 NFAT response elements. The NFAT pathway was activated by the addition of PMA at 10 ng/mL and lonomycin at either 1 uM or 2 uM, except in control (ctl) cells. Pulldown was done using beads to which were coupled an antibody that recognizes a Flag tag that was incorporated at the C-terminus of the MMP9 construct. Lane 1 shows a molecular weight control. Lanes 2, 3, 4 and 5 show MMP9 that was eluted from the anti-Flag tag beads. Lanes 2 and 3 were first elutions and the cells shown in Lanes 4 and 5 were second elutions. Into Lanes 2 and 4 were loaded conditioned media from cells in which the NFAT pathway had been activated with PMA 10 ng/mL and lonomycin at 1 uM. Into Lanes 3 and 5 were loaded conditioned media from cells in which the NFAT pathway had been activated with PMA 10 ng/mL and lonomycin at 2 uM.

FIGS. 100A-100E show NFAT-induced MMP9 catalytic domain expressed in HEK293T cells wherein the native leader sequence of MMP9 has been replaced by an IgK leader sequence and MMP9 catalytic domain is downstream of 4 repeats of an NFAT response element. FIG. 100A shows photograph of Western blot detecting expression of MMP9 in the cell lysate after activation of the NFAT pathway. FIG. 100B shows photograph of Western blot detecting expression of MMP9 in the conditioned media after activation of the NFAT pathway.

FIGS. 101A-101E show MMP9 can be expressed with different leader sequences and also show subsequent activity of each. FIG. 101A shows a Western blot detecting an MMP9 protein in cell lysate wherein the leader sequence upstream of the MMP9 gene is either its native sequence or an IgK sequence. FIG. 101B shows a Western blot detecting MMP9 in conditioned media wherein the leader sequence upstream of the MMP9 gene is its native sequence or an IgK.

FIGS. 102A-102D show three (3) clones 4, 6 and 7 of cells transfected with a plasmid that produces an NFAT inducible MMP9 wherein the NFATc1 promoter sequence is upstream of the MMP9 gene, which in this case is a truncated MMP9 comprising its catalytic domain. Also shown for comparison is a cell transfected with a plasmid that produces an NFAT inducible MMP9 wherein 4 repeats of an NFAT response element sequence are upstream of an MMP9 gene. FIG. 102A shows a Western blot detecting an MMP9 protein in cell lysate. FIG. 102B shows a Western blot detecting MMP9 in the conditioned media.

FIGS. 105A-105E show photographs of Western blots of human T cells transduced with either CAR44 alone, NFAT-inducible MMP9 alone or transduced with both CAR44 and NFAT-inducible MMP9, wherein the resultant T cells are either not activated, chemically activated by PMA/Ionomycin, activated by co-culturing with beads presenting synthetic MUC1* peptide or co-culturing with MUC1* positive cancer cells. Western blot was probed with an anti-Flag tag also known as DYK tag antibody. Catalytic domain of MMP9 runs with an apparent molecular weight of about 40 kDa. FIGS. 105A-105D show photographs of Western blots of cleared cell lysates. Results show that T cells transduced with NFAT-inducible MMP9 only express MMP9 when they are activated by PMA/Ionomycin, MUC1* beads or MUC1* positive cancer cells. T cells transduced with both CAR44 and NFAT-inducible MMP9 only express MMP9 when the T cells are activated by stimulation with MUC1* beads or with MUC1* positive cancer cells.

Results show that T cells transduced with NFAT-inducible MMP9 express MMP9 when they are activated. T cells transduced with both CAR44 and NFAT-inducible MMP9 are specifically activated when they are co-cultured with beads or cells presenting or expressing MUC1* (FIG. 105A Lane 5 and Lane 6).

10. Fluorogenic MMP Peptide Substrate Cleavage Assay

OMNIMMP fluorogenic substrate (Enzo life sciences, NY) was diluted to 20 uM in assay buffer (50 mM Tris pH 7.5, 300 mM NaCl, 1 mM CaCl2, 5 uM Zncl2, 0.1% Brj-35 and 15% glycerol) and kept on ice and protected from light until used. Peptide can also be diluted in PBS pH 7.4 or culture medium. Cell lysate was diluted to 0.4 mg/mL is assay buffer (or PBS pH 7.4 or culture medium). For the assay, 50 uL of recombinant MMP9 catalytic domain (1-2 ug/mL in assay buffer, PBS pH 7.4 or culture medium), 50 uL of diluted cell lysate, 50 uL of conditioned media or 50 uL of pulled down protein was added to wells of a 96 well plate compatible with fluorometer. Just before starting the assay, 50 uL of diluted peptide was added to each well and quickly mixed (final peptide concentration is 10 uM). Fluorescence was recorded every 10 min for about 6 h at 37° C. (Ex.: 328 nm, Em.: 393 nm).

FIG. 97 shows a graph of a fluorogenic peptide substrate of MMP9, the OMNIMMP peptide, being cleaved by MMP9 catalytic domain at two concentrations in either PBS, solid trace, or cell culture media, dashed trace.

FIGS. 99A-99C show graphs of a fluorogenic peptide, OMNIMMP peptide, substrate of MMP9 being cleaved by the cell lysate or conditioned media of HEK293T cells that were transfected with a plasmid containing an MMP9 gene downstream from 4 repeats of an NFAT response element. The MMP9 peptide substrate assay shows that activation of the NFAT pathway by PMA/ionomycin caused an MMP9 to be expressed and secreted and that it was active as evidenced by its ability to cleave a peptide substrate.

FIG. 100C shows graph of MMP9 fluorogenic peptide substrate, OMNIMMP peptide, cleavage by MMP9 catalytic domain expressed and secreted in conditioned media of HEK293T cells wherein the native leader sequence of MMP9 has been replaced by an IgK leader sequence and MMP9 catalytic domain is downstream of 4 repeats of an NFAT response element. FIG. 100D shows graph of MMP9 fluorogenic peptide substrate cleavage by MMP9 catalytic domain expressed and secreted in conditioned media of HEK293T cells wherein the native leader sequence of MMP9 has been replaced by an IgK leader sequence and MMP9 catalytic domain is downstream of 4 repeats of an NFAT response element.

FIG. 101C shows a graph of an MMP9 peptide substrate cleaved by the expressed MMP9.

FIGS. 103A-103D show graphs of an MMP9 peptide substrate cleavage assay. FIG. 103A shows the cleavage activity of MMP9 from the lysate of cells transfected with a plasmid having MMP9 expression driven from the NFATc1 promoter or from 4 repeats of an NFAT response element. FIG. 103B shows the cleavage activity of MMP9 from the conditioned media of cells transfected with a plasmid having MMP9 expression driven from the NFATc1 promoter or off of 4 repeats of an NFAT response element.

FIGS. 104A-104B show the results of the OMNIMMP9 fluorogenic substrate assay that measures activity of MMP9. Conditioned media from human T cells transduced with NFAT-inducible MMP9 alone or in combination with CAR44 were added to the assay and MMP9 substrate cleavage was measured as a function of time. FIG. 104A shows MMP9 activity when human T cells were transduced with both CAR44 and an NFAT-inducible MMP9 after the cells were activated by co-culturing with HCT-MUC1* cancer cells. The trace that does not show increased substrate cleavage as a function of time is the conditioned media from cells that were not activated. FIG. 104B shows MMP9 activity when human T cells were transduced with just an NFAT-inducible MMP9 after the cells were activated by co-culturing with beads coated with anti-CD3 and anti-CD28 which are known to activate T cells. The trace that does not show increased substrate cleavage as a function of time is the conditioned media from cells that were not activated.

11. Cloning

MMP9 Catalytic Domain Cloning in Lentivector Downstream of NFAT Response Elements:

Two sequences were synthesized (pNFAT-MMP9cat-1 and pNFAT-MMP9cat-2, (SEQ ID NO:784 and SEQ ID NO:785). The lentivector pGreenFire1-4×NFAT (System Biosciences, CA) was digested with SpeI and KpnI restriction enzymes (New England Biolabs). The purified fragment and the 2 synthesized sequences were assembled using the Gibson assembly cloning kit (New England Biolab). The resulting constructs (pGreenFire1-4×NFAT-MMP9cat) contains 4 repeats of a NFAT response element followed by a minimum promoter (mCMV) and the MMP9 catalytic domain with its native leader sequence.

Cloning of NFAT Response Element in pGL4-14[Luc2/Hygro]:

The 4×NFAT domain was amplified from the lentivector pGreenFire1-4×NFAT by polymerase chain reaction (PCR) using the following primer: 5′-tagatggtaccaagaggaaaatttgtttcatacag-3′ (SEQ ID NO: 786) and 5′-tagataagcttgctggatcggtcccggtgtc-3′ (SEQ ID NO: 787). After digestion with KpnI and HindIII restriction enzymes (New England Biolabs), the purified fragment was cloned into the promoter-less vector pGL4-14[luc2/Hygro](Promega) digested with the same restriction enzymes to create the construct pGL4-14-4×NFAT.

Cloning of MMP9 Catalytic Domain into pGL4-14-4×NFAT:

A fragment containing a minimum promoter (mCMV) followed by MMP9 native leader sequence and MMP9 catalytic domain was amplified from the lentivector pGreenFire1-4×NFAT-MMP9cat by polymerase chain reaction (PCR) using the following primer: 5′-tcatacagaaggcgttactagttaggcgtgtacggtgg-3′ (SEQ ID NO:788) and 5′-acagtaccggattgccaagcttttatcacttatcgtcgtcatccttg-3′ (SEQ ID NO:789). pGL4-14-4×NFAT was digested with SpeI and HindIII restriction enzymes (New England Biolabs). The purified PCR fragment and digested pGL4-14-4×NFAT were assembled using the Gibson assembly cloning kit (New England Biolab) to create the construct pGL4-14-4×NFAT-MMP9cat.

Cloning of MMP9 Catalytic Domain into pSECTag2:

MMP9 catalytic domain without its native leader sequence was amplified from the lentivector pGreenFire1-4×NFAT-MMP9cat by polymerase chain reaction (PCR) using the following primer: 5′-aagttggtaccgttccaaacctttgagggcgacc-3′ (SEQ ID NO:790) and 5′-aagttctcgagcaggttcagggcgaggaccatag-3′ (SEQ ID NO:791). After digestion with KpnI and XhoI restriction enzymes (New England Biolabs), the purified fragment was cloned into the vector pSECTag2 A (ThermoFisher Scientific) digested with the same restriction enzymes to create the construct pSECTag2 MMP9 cat His. In this construct MMP9 catalytic domain will downstream if the IgK leader sequence.

Cloning of MMP9 Catalytic Domain with IgK Leader Sequence into pGL4-14-4×NFAT:

MMP9 catalytic domain with its native leader sequence was amplified from the pGL4-14-4×NFAT-MMP9cat by polymerase chain reaction (PCR) using the following primer: 5′-attgactcgagctctcgacattcgtttctagagc-3′ (SEQ ID NO:792) and 5′-attgaaagcttttatcacttatcgtcgtcatccttg-3′ (SEQ ID NO:793). After digestion with XhoI and HindIII restriction enzymes (New England Biolabs), the purified fragment was cloned into the vector pGL4-14[luc2/Hygro] (Promega) digested with the same restriction enzymes to create the construct pGL4-14 MMP9cat XH.

A fragment containing 4×NFAT response elements followed by the minimum promoter (mCMV) was amplified from pGL4-14-4×NFAT-MMP9cat by polymerase chain reaction (PCR) using the following primer: 5′-tagcaaaataggctgtccc-3′ (SEQ ID NO:794) and 5′-attgactcgaggctggatcggtcccggtgtc-3′ (SEQ ID NO:795). After digestion with KpnI and XhoI restriction enzymes (New England Biolabs), the purified fragment was cloned into the vector pGL4-14 MMP9cat XH digested with the same restriction enzymes to create the construct pGL4-14 4×NFAT-MMP9cat KXH

A fragment containing the IgK leader sequence followed by MMP9 catalytic domain was amplified from pSECTag2 MMP9 cat by polymerase chain reaction (PCR) using the following primer: 5′-aagacaccgggaccgatccagcctcgagagacccaagctggctagccacc-3′ (SEQ ID NO:796) and 5′-ttaccaacagtaccggattgccaagcttttatcacttatcgtcgtcatcc-3′ (SEQ ID NO:797). pGL4-14 4×NFAT-MMP9cat KXH was digested with XhoI and HindIII restriction enzymes (New England Biolabs). The purified PCR fragment and digested pGL4-14 4×NFAT-MMP9cat KXH were assembled using the Gibson assembly cloning kit (New England Biolab) to create the construct pGL4-14-4×NFAT-IgK MMP9cat.

Cloning of MMP9 Catalytic Domain into pEZX-PG02.1 Downstream of NFATc1 Promoter:

MMP9 catalytic domain with its native leader sequence was amplified from the lentivector pGreenFire1-4×NFAT-MMP9cat by polymerase chain reaction (PCR) using the following primer: 5′-attgaaagcttctctcgacattcgtttctagagc-3′ (SEQ ID NO:798) and 5′-attgagagctcttatcacttatcgtcgtcatc-3′ (SEQ ID NO:799). After digestion with HindIII and SacI restriction enzymes (New England Biolabs), the purified fragment was cloned into the vector pEZX-PG02.1 downstream of the NFACTc1 promoter (GeneCopoeia, MD) to create the construct pEZX-NFATc 1-MMP9cat.

Modification of pEZX-NFATc1-MMP9cat:

pEZX-NFATc1-MMP9cat was modified to introduce SpeI and KpnI restriction site 5′ of the NFATc1 promoter and NheI and EcoRV restriction site 3′ of MMP9 catalytic domain. Two gBLOCKs were synthesized by our request by IDT, IA. (NFAT modif 1 and NFAT modif 2, SEQ ID NO:800 and SEQ ID NO:801). The pEZX-NFATc1-MMP9cat vector was digested with NheI, EcoRI, SacI and XhoI restriction enzymes (New England Biolabs). Two fragments were purified and assembled with the two synthesized gBLOCKS using the Gibson assembly cloning kit (New England Biolab).

Cloning of NFATc1 Promoter/MMP9 Catalytic Domain into Lentivector pCDH-CMV-MCS-EF1α-Hygro:

Modified pEZX-NFATc1-MMP9cat vector was digested with SpeI and NheI restriction enzymes (New England Biolabs) and the fragment containing NFATc1 promoter followed by MMP9 catalytic domain was purified and cloned into the lentivector pCDH-CMV-MCS-EF1α-Hygro (System Biosciences) digested with the same restriction enzymes.

Cloning of NFAT Response Element/MMP9 Catalytic Domain into Lentivector pCDH-CMV-MCS-EF1α-Hygro:

A fragment containing 4 repeats of a NFAT response element followed by MMP9 catalytic domain with its native leader sequence was amplified from the vector pGL4-14-4×NFAT-MMP9cat by polymerase chain reaction (PCR) using the following primer: 5′-acaaaattcaaaattttatcgatactagttggcctaactggccggtaccaag-3′ (SEQ ID NO:802) and 5′-atccgatttaaattcgaattcgctagcttatcacttatcgtcgtcatcc-3′ (SEQ ID NO:803). The purified PCR fragment and digested pCDH-CMV-MCS-EF1α-Hygro (SpeI and NheI) were assembled using the Gibson assembly cloning kit (New England Biolab).

All of the references cited herein are incorporated by reference in their entirety.

Sequence Listing Free Text
As regards the use of nucleotide symbols other than a, g, c, t, they follow the
convention set forth in WIPO Standard ST.25, Appendix 2, Table 1, wherein k
represents t or g; n represents a, c, t or g; m represents a or c; r represents
a or g; s represents c or g; w represents a or t and y represents c or t.
MUC1 Receptor
(Mucin 1 precursor, Genbank Accession number: P15941)
(SEQ ID NO: 1)
MTPGTQSPFFLLLLLTVLTVVTGSGHASSTPGGEKETSATQRSSVPSSTEKNAVSMTSSVLSSHSPGSGSSTTQGQDVTLA
PATEPASGSAATWGQDVTSVPVTRPALGSTTPPAHDVTSAPDNKPAPGSTAPPAHGVTSAPDTRPAPGSTAPPAHGVTSAP
DTRPAPGSTAPPAHGVTSAPDTRPAPGSTAPPAHGVTSAPDTRPAPGSTAPPAHGVTSAPDTRPAPGSTAPPAHGVTSAPD
TRPAPGSTAPPAHGVTSAPDTRPAPGSTAPPAHGVTSAPDTRPAPGSTAPPAHGVTSAPDTRPAPGSTAPPAHGVTSAPDT
RPAPGSTAPPAHGVTSAPDTRPAPGSTAPPAHGVTSAPDTRPAPGSTAPPAHGVTSAPDTRPAPGSTAPPAHGVTSAPDTR
PAPGSTAPPAHGVTSAPDTRPAPGSTAPPAHGVTSAPDTRPAPGSTAPPAHGVTSAPDTRPAPGSTAPPAHGVTSAPDTRP
APGSTAPPAHGVTSAPDTRPAPGSTAPPAHGVTSAPDTRPAPGSTAPPAHGVTSAPDTRPAPGSTAPPAHGVTSAPDTRPA
PGSTAPPAHGVTSAPDTRPAPGSTAPPAHGVTSAPDTRPAPGSTAPPAHGVTSAPDTRPAPGSTAPPAHGVTSAPDTRPAP
GSTAPPAHGVTSAPDTRPAPGSTAPPAHGVTSAPDTRPAPGSTAPPAHGVTSAPDTRPAPGSTAPPAHGVTSAPDTRPAPG
STAPPAHGVTSAPDTRPAPGSTAPPAHGVTSAPDTRPAPGSTAPPAHGVTSAPDTRPAPGSTAPPAHGVTSAPDTRPAPGS
TAPPAHGVTSAPDTRPAPGSTAPPAHGVTSAPDTRPAPGSTAPPAHGVTSAPDTRPAPGSTAPPAHGVTSAPDTRPAPGST
APPAHGVTSAPDTRPAPGSTAPPAHGVTSAPDTRPAPGSTAPPAHGVTSAPDNRPALGSTAPPVHNVTSASGSASGSASTL
VHNGTSARATTTPASKSTPFSIPSHHSDTPTTLASHSTKTDASSTHHSSVPPLTSSNHSTSPQLSTGVSFFFLSFHISNLQ
FNSSLEDPSTDYYQELQRDISEMFLQIYKQGGFLGLSNIKFRPGSVVVQLTLAFREGTINVHDVETQFNQYKTEAASRYNL
TISDVSVSDVPFPFSAQSGAGVPGWGIALLVLVCVLVALAIVYLIALAVCQCRRKNYGQLDIFPARDTYHPMSEYPTYHTH
GRYVPPSSTDRSPYEKVSAGNGGSSLSYTNPAVAAASANL
PSMGFR
(SEQ ID NO: 2)
GTINVHDVETQFNQYKTEAASRYNLTISDVSVSDVPFPFSAQSGA
Human NME1
(DNA)
(SEQ ID NO: 3)
atggccaactgtgagcgtaccttcattgcgatcaaaccagatggggtccagcggggtcttgtgggagagattatcaagcg
ttttgagcagaaaggattccgccttgttggtctgaaattcatgcaagcttccgaagatcttctcaaggaacactacgttg
acctgaaggaccgtccattctttgccggcctggtgaaatacatgcactcagggccggtagttgccatggtctgggagggg
ctgaatgtggtgaagacgggccgagtcatgctcggggagaccaaccctgcagactccaagcctgggaccatccgtggaga
cttctgcatacaagttggcaggaacattatacatggcagtgattctgtggagagtgcagagaaggagatcggcttgtggt
ttcaccctgaggaactggtagattacacgagctgtgctcagaactggatctatgaatga
(amino acids)
(SEQ ID NO: 4)
MANCERTFIAIKPDGVQRGLVGEIIKRFEQKGFRLVGLKFMQASEDLLKEHYVDLKDRPFFAGLVKYMHSGPVVAMVWEG
LNVVKTGRVMLGETNPADSKPGTIRGDFCIQVGRNIIHGSDSVESAEKEIGLWFHPEELVDYTSCAQNWIYE-
Human NME7
(DNA)
(SEQ ID NO: 5)
atgaatcatagtgaaagattcgttttcattgcagagtggtatgatccaaatgcttcacttcttcgacgttatgagctttt
attttacccaggggatggatctgttgaaatgcatgatgtaaagaatcatcgcacctttttaaagcggaccaaatatgata
acctgcacttggaagatttatttataggcaacaaagtgaatgtcttttctcgacaactggtattaattgactatggggat
caatatacagctcgccagctgggcagtaggaaagaaaaaacgctagccctaattaaaccagatgcaatatcaaaggctgg
agaaataattgaaataataaacaaagctggatttactataaccaaactcaaaatgatgatgctttcaaggaaagaagcat
tggattttcatgtagatcaccagtcaagaccctttttcaatgagctgatccagtttattacaactggtcctattattgcc
atggagattttaagagatgatgctatatgtgaatggaaaagactgctgggacctgcaaactctggagtggcacgcacaga
tgcttctgaaagcattagagccctctttggaacagatggcataagaaatgcagcgcatggccctgattcttttgcttctg
cggccagagaaatggagttgttttttccttcaagtggaggttgtgggccggcaaacactgctaaatttactaattgtacc
tgttgcattgttaaaccccatgctgtcagtgaaggactgttgggaaagatcctgatggctatccgagatgcaggttttga
aatctcagctatgcagatgttcaatatggatcgggttaatgttgaggaattctatgaagtttataaaggagtagtgaccg
aatatcatgacatggtgacagaaatgtattctggcccttgtgtagcaatggagattcaacagaataatgctacaaagaca
tttcgagaattttgtggacctgctgatcctgaaattgcccggcatttacgccctggaactctcagagcaatctttggtaa
aactaagatccagaatgctgttcactgtactgatctgccagaggatggcctattagaggttcaatacttcttcaagatct
tggataattag
(amino acids)
(SEQ ID NO: 6)
MNHSERFVFIAEWYDPNASLLRRYELLFYPGDGSVEMHDVKNHRTFLKRTKYDNLHLEDLFIGNKVNVFSRQLVLIDYGD
QYTARQLGSRKEKTLALIKPDAISKAGEIIEIINKAGFTITKLKMMMLSRKEALDFHVDHQSRPFFNELIQFITTGPIIA
MEILRDDAICEWKRLLGPANSGVARTDASESIRALFGTDGIRNAAHGPDSFASAAREMELFFPSSGGCGPANTAKFTNCT
CCIVKPHAVSEGLLGKILMAIRDAGFEISAMQMFNMDRVNVEEFYEVYKGVVTEYHDMVTEMYSGPCVAMEIQQNNATKT
FREFCGPADPEIARHLRPGTLRAIFGKTKIQNAVHCTDLPEDGLLEVQYFFKILDN-
NME7 peptides
NME7A peptide 1 (A domain):
(SEQ ID NO: 7)
MLSRKEALDFHVDHQS
NME7A peptide 2 (A domain):
(SEQ ID NO: 8)
SGVARTDASES
NME7B peptide 1 (B domain):
(SEQ ID NO: 9)
DAGFEISAMQMFNMDRVNVE
NME7B peptide 2 (B domain):
(SEQ ID NO: 10)
EVYKGVVTEYHDMVTE
NME7B peptide 3 (B domain):
(SEQ ID NO: 11)
AIFGKTKIQNAVHCTDLPEDGLLEVQYFF
Mouse E6 Heavy chain variable region sequence:
(DNA)
(SEQ ID NO: 12)
gaggtgaaggtggtggagtctgggggagacttagtgaagcctggagggtccctgaaactctcctgtgtagtctctggattc
actttcagtagatatggcatgtcttgggttcgccagactccaggcaagaggctggagtgggtcgcaaccattagtggtggc
ggtacttacatctactatccagacagtgtgaaggggcgattcaccatctccagagacaatgccaagaacaccctgtacctg
caaatgagcagtctgaagtctgaggacacagccatgtatcactgtacaagggataactacggtaggaactacgactacggt
atggactactggggtcaaggaacctcagtcaccgtctcctca
(amino acids)
(SEQ ID NO: 13)
EVKVVESGGDLVKPGGSLKLSCVVSGFTFSRYGMSWVRQTPGKRLEWVATISGGGTYIYYPDSVKGRFTISRDNAKNTLYL
QMSSLKSEDTAMYHCTRDNYGRNYDYGMDYWGQGTSVTVSS
Mouse E6 heavy chain variable framework region 1 (FWR1) sequence:
(DNA)
(SEQ ID NO: 14)
gaggtgaaggtggtggagtctgggggagacttagtgaagcctggagggtccctgaaactctcctgtgtagtctct
(amino acids)
(SEQ ID NO: 15)
EVKVVESGGDLVKPGGSLKLSCVVSGFTFS
Mouse E6 heavy chain variable complementarity determining regions 1 (CDR1)
sequence:
(DNA)
(SEQ ID NO: 16)
ggattcactttcagtagatatggcatgtct
(amino acids)
(SEQ ID NO: 17)
RYGMS
Mouse E6 heavy chain variable framework region 2 (FWR2) sequence:
(DNA)
(SEQ ID NO: 18)
tgggttcgccagactccaggcaagaggctggagtgggtcgca
(amino acids)
(SEQ ID NO: 19)
WVRQTPGKRLEWVA
Mouse E6 heavy chain variable complementarity determining regions 2 (CDR2)
sequence:
(DNA)
(SEQ ID NO: 20)
accattagtggtggcggtacttacatctactatccagacagtgtgaagggg
(amino acids)
(SEQ ID NO: 21)
TISGGGTYIYYPDSVKG
Mouse E6 heavy chain variable framework region 3 (FWR3) acid sequence:
(DNA)
(SEQ ID NO: 22)
cgattcaccatctccagagacaatgccaagaacaccctgtacctgcaaatgagcagtctgaagtctgaggacacagccat
gtatcactgtacaagg
(amino acids)
(SEQ ID NO: 23)
RFTISRDNAKNTLYLQMSSLKSEDTAMYHCTR
Mouse E6 heavy chain variable complementarity determining regions 3 (CDR3)
sequence:
(DNA)
(SEQ ID NO: 24)
gataactacggtaggaactacgactacggtatggactac
(amino acids)
(SEQ ID NO: 25)
DNYGRNYDYGMDY
IGHV3-21*03 heavy chain variable region sequence:
(DNA)
(SEQ ID NO: 26)
gaggtgcagctggtggagtctgggggaggcctggtcaagcctggggggtccctgagactctcctgtgcagcctctggatt
caccttcagtagctatagcatgaactgggtccgccaggctccagggaaggggctggagtgggtctcatccattagtagta
gtagtagttacatatactacgcagactcagtgaagggccgattcaccatctccagagacaacgccaagaactcactgtat
ctgcaaatgaacagcctgagagccgaggacacggctgtgtattactgtgcgaga
(amino acids)
(SEQ ID NO: 27)
EVQLVESGGGLVKPGGSLRLSCAASGFTFSSYSMNWVRQAPGKGLEWVSSISSSSSYIYYADSVKGRFTISRDNAKNSLY
LQMNSLRAEDTAVYYCAR
IGHV3-21*01 heavy chain variable framework region 1 (FWR1) sequence:
(DNA)
(SEQ ID NO: 28)
gaggtgcagctggtggagtctgggggaggcctggtcaagcctggggggtccctgagactctcctgtgcagcctctggatt
caccttcagt
(amino acids)
(SEQ ID NO: 29)
EVQLVESGGGLVKPGGSLRLSCAASGFTFS
IGHV3-21*01 heavy chain variable complementarity determining regions 1 (CDR1)
sequence:
(DNA)
(SEQ ID NO: 30)
agctatagcatgaac
(amino acids)
(SEQ ID NO: 31)
SYSMN
IGHV3-21*01 heavy chain variable framework region 2 (FWR2) sequence:
(DNA)
(SEQ ID NO: 32)
tgggtccgccaggctccagggaaggggctggagtgggtctca
(amino acids)
(SEQ ID NO: 33)
WVRQAPGKGLEWVS
IGHV3-21*01 heavy chain variable complementarity determining regions 2 (CDR2)
sequence:
(DNA)
(SEQ ID NO: 34)
tccattagtagtagtagtagttacatatactacgcagactcagtgaagggc
(amino acids)
(SEQ ID NO: 35)
SISSSSSYIYYADSVKG
IGHV3-21*01 heavy chain variable framework region 3 (FWR3) sequence:
(DNA)
(SEQ ID NO: 36)
cgattcaccatctccagagacaacgccaagaactcactgtatctgcaaatgaacagcctgagagccgaggacacggctgt
gtattactgtgcgaga
(amino acids)
(SEQ ID NO: 37)
RFTISRDNAKNSLYLQMNSLRAEDTAVYYCAR
Humanized E6 heavy chain variable region sequence:
(DNA)
(SEQ ID NO: 38)
gaggtgcagctggtggagtctgggggaggcctggtcaagcctggggggtccctgagactctcctgtgcagcctctggatt
caccttcagtaggtatggcatgagctgggtccgccaggctccagggaagaggctggagtgggtctcaaccattagtggcg
gaggcacctacatatactacccagactcagtgaagggccgattcaccatctccagagacaacgccaagaacaccctgtat
ctgcaaatgaacagcctgagagccgaggacacggctgtgtattactgtaccagagataactatggccgcaactatgatta
tggcatggattattggggccagggcaccctggtgaccgtgagcagc
(amino acids)
(SEQ ID NO: 39)
EVQLVESGGGLVKPGGSLRLSCAASGFTFSRYGMSWVRQAPGKRLEWVSTISGGGTYIYYPDSVKGRFTISRDNAKNTLY
LQMNSLRAEDTAVYYCTRDNYGRNYDYGMDYWGQGTLVTVSS
Humanized E6 heavy chain variable framework region 1 (FWR1) acid sequence:
(DNA)
(SEQ ID NO: 40)
gaggtgcagctggtggagtctgggggaggcctggtcaagcctggggggtccctgagactctcctgtgcagcctctggatt
caccttcagt
(amino acids)
(SEQ ID NO: 41)
EVQLVESGGGLVKPGGSLRLSCAASGFTFS
Humanized E6 heavy chain variable complementarity determining regions 1 (CDR1)
sequence:
(DNA)
(SEQ ID NO: 42)
aggtatggcatgagc
(amino acids)
(SEQ ID NO: 43)
RYGMS
Humanized E6 heavy chain variable framework region 2 (FWR2) acid sequence:
(DNA)
(SEQ ID NO: 44)
tgggtccgccaggctccagggaagaggctggagtgggtctca
(amino acids)
(SEQ ID NO: 45)
WVRQAPGKRLEWVS
Humanized E6 heavy chain variable complementarity determining regions 2 (CDR2)
sequence:
(DNA)
(SEQ ID NO: 46)
accattagtggcggaggcacctacatatactacccagactcagtgaagggc
(amino acids)
(SEQ ID NO: 47)
TISGGGTYIYYPDSVKG
Humanized E6 heavy chain variable framework region 3 (FWR3) acid sequence:
(DNA)
(SEQ ID NO: 48)
cgattcaccatctccagagacaacgccaagaacaccctgtatctgcaaatgaacagcctgagagccgaggacacggctgtg
tattactgtaccaga
(amino acids)
(SEQ ID NO: 49)
RFTISRDNAKNTLYLQMNSLRAEDTAVYYCTR
Humanized E6 heavy chain variable complementarity determining regions 3 (CDR3)
sequence:
(DNA)
(SEQ ID NO: 50)
gataactatggccgcaactatgattatggcatggattat
(amino acids)
(SEQ ID NO: 51)
DNYGRNYDYGMDY
Humanized E6 IgG2 heavy chain synthesized by Genescript:
(DNA)
(SEQ ID NO: 52)
gaattctaagcttgggccaccatggaactggggctccgctgggttttccttgttgctattttagaaggtgtccagtgtgag
gtgcagctggtggagtctgggggaggcctggtcaagcctggggggtccctgagactctcctgtgcagcctctggattcacc
ttcagtaggtatggcatgagctgggtccgccaggctccagggaagaggctggagtgggtctcaaccattagtggcggaggc
acctacatatactacccagactcagtgaagggccgattcaccatctccagagacaacgccaagaacaccctgtatctgcaa
atgaacagcctgagagccgaggacacggctgtgtattactgtaccagagataactatggccgcaactatgattatggcatg
gattattggggccagggcaccctggtgaccgtgagcagcgcctccaccaagggcccatcggtcttccccctggcgccctgc
tccaggagcacctccgagagcacagccgccctgggctgcctggtcaaggactacttccccgaaccggtgacggtgtcgtgg
aactcaggcgctctgaccagcggcgtgcacaccttcccagctgtcctacagtcctcaggactctactccctcagcagcgtg
gtgaccgtgccctccagcaacttcggcacccagacctacacctgcaacgtagatcacaagcccagcaacaccaaggtggac
aagacagttgagcgcaaatgttgtgtcgagtgcccaccgtgcccagcaccacctgtggcaggaccgtcagtcttcctcttc
cccccaaaacccaaggacaccctcatgatctcccggacccctgaggtcacgtgcgtggtggtggacgtgagccacgaagac
cccgaggtccagttcaactggtacgtggacggcgtggaggtgcataatgccaagacaaagccacgggaggagcagttcaac
agcacgttccgtgtggtcagcgtcctcaccgttgtgcaccaggactggctgaacggcaaggagtacaagtgcaaggtctcc
aacaaaggcctcccagcccccatcgagaaaaccatctccaaaaccaaagggcagccccgagaaccacaggtgtacaccctg
cccccatcccgggaggagatgaccaagaaccaggtcagcctgacctgcctggtcaaaggcttctaccccagcgacatcgcc
gtggagtgggagagcaatgggcagccggagaacaactacaagaccacacctcccatgctggactccgacggctccttcttc
ctctacagcaagctcaccgtggacaagagcaggtggcagcaggggaacgtcttctcatgctccgtgatgcatgaggctctg
cacaaccactacacgcagaagagcctctccctgtctccgggtaaatagtaagtttaaactctaga
(amino acids)
(SEQ ID NO: 53)
EF*AWATMELGLRWVFLVAILEGVQCEVQLVESGGGLVKPGGSLRLSCAASGFTFSRYGMSWVRQAPGKRLEWVSTISGGG
TYIYYPDSVKGRFTISRDNAKNTLYLQMNSLRAEDTAVYYCTRDNYGRNYDYGMDYWGQGTLVTVSSASTKGPSVFPLAPC
SRSTSESTAALGCLVKDYFPEPVTVSWNSGALTSGVHTFPAVLQSSGLYSLSSVVTVPSSNFGTQTYTCNVDHKPSNTKVD
KTVERKCCVECPPCPAPPVAGPSVFLFPPKPKDTLMISRTPEVTCVVVDVSHEDPEVQFNWYVDGVEVHNAKTKPREEQFN
STFRVVSVLTVVHQDWLNGKEYKCKVSNKGLPAPIEKTISKTKGQPREPQVYTLPPSREEMTKNQVSLTCLVKGFYPSDIA
VEWESNGQPENNYKTTPPMLDSDGSFFLYSKLTVDKSRWQQGNVFSCSVMHEALHNHYTQKSLSLSPGK**V*TLX
Human IgG2 heavy chain constant region sequence:
(DNA)
(SEQ ID NO: 54)
gcctccaccaagggcccatcggtcttccccctggcgccctgctccaggagcacctccgagagcacagccgccctgggctgc
ctggtcaaggactacttccccgaaccggtgacggtgtcgtggaactcaggcgctctgaccagcggcgtgcacaccttccca
gctgtcctacagtcctcaggactctactccctcagcagcgtggtgaccgtgccctccagcaacttcggcacccagacctac
acctgcaacgtagatcacaagcccagcaacaccaaggtggacaagacagttgagcgcaaatgttgtgtcgagtgcccaccg
tgcccagcaccacctgtggcaggaccgtcagtcttcctcttccccccaaaacccaaggacaccctcatgatctcccggacc
cctgaggtcacgtgcgtggtggtggacgtgagccacgaagaccccgaggtccagttcaactggtacgtggacggcgtggag
gtgcataatgccaagacaaagccacgggaggagcagttcaacagcacgttccgtgtggtcagcgtcctcaccgttgtgcac
caggactggctgaacggcaaggagtacaagtgcaaggtctccaacaaaggcctcccagcccccatcgagaaaaccatctcc
aaaaccaaagggcagccccgagaaccacaggtgtacaccctgcccccatcccgggaggagatgaccaagaaccaggtcagc
ctgacctgcctggtcaaaggcttctaccccagcgacatcgccgtggagtgggagagcaatgggcagccggagaacaactac
aagaccacacctcccatgctggactccgacggctccttcttcctctacagcaagctcaccgtggacaagagcaggtggcag
caggggaacgtcttctcatgctccgtgatgcatgaggctctgcacaaccactacacgcagaagagcctctccctgtctccg
ggtaaatag
(amino acids)
(SEQ ID NO: 55)
ASTKGPSVFPLAPCSRSTSESTAALGCLVKDYFPEPVTVSWNSGALTSGVHTFPAVLQSSGLYSLSSVVTVPSSNFGTQTY
TCNVDHKPSNTKVDKTVERKCCVECPPCPAPPVAGPSVFLFPPKPKDTLMISRTPEVTCVVVDVSHEDPEVQFNWYVDGVE
VHNAKTKPREEQFNSTFRVVSVLTVVHQDWLNGKEYKCKVSNKGLPAPIEKTISKTKGQPREPQVYTLPPSREEMTKNQVS
LTCLVKGFYPSDIAVEWESNGQPENNYKTTPPMLDSDGSFFLYSKLTVDKSRWQQGNVFSCSVMHEALHNHYTQKSLSLSP
GK
Humanized E6 IgG1 heavy chain sequence:
(DNA)
(SEQ ID NO: 56)
gaggtgcagctggtggagtctgggggaggcctggtcaagcctggggggtccctgagactctcctgtgcagcctctggattc
accttcagtaggtatggcatgagctgggtccgccaggctccagggaagaggctggagtgggtctcaaccattagtggcgga
ggcacctacatatactacccagactcagtgaagggccgattcaccatctccagagacaacgccaagaacccactgtatctg
caaatgaacagcctgagagccgaggacacggctgtgtattactgtcccagagataactatggccgcaactatgattatggc
atggattattggggccagggcaccctggtgaccgtgagcagcgctagcaccaagggcccatcggtcttccccctggcaccc
tcctccaagagcacctctgggggcacagcggccctgggctgcctggtcaaggactacttccccgaaccggtgacggtgtcg
tggaactcaggcgccctgaccagcggcgtgcacaccttcccggctgtcctacagtcctcaggactctactccctcagcagc
gtggtgacagtgccctccagcagcttgggcacccagacctacatctgcaacgtgaatcacaagcccagcaacaccaaggtg
gacaagaaagttgagcccaaatcttgtgacaaaactcacacatgcccaccgtgcccagcacctgaactcctggggggaccg
tcagtcttcctcttccccccaaaacccaaggacaccctcatgatctcccggacccctgaggtcacatgcgtggtggtggac
gtgagccacgaagaccctgaggtcaagttcaactggtacgtggacggcgtggaggtgcataatgccaagacaaagccgcgg
gaggagcagtacaacagcacgtaccgtgtggtcagcgtcctcaccgtcctgcaccaggactggctgaatggcaaggagtac
aagtgcaaggtctccaacaaagccctcccagcccccatcgagaaaaccatctccaaagccaaagggcagccccgagaacca
caggtgtacaccctgcccccatcccgggaggagatgaccaagaaccaggtcagcctgacctgcctggtcaaaggcttctat
cccagcgacatcgccgtggagtgggagagcaatgggcagccggagaacaactacaagaccacgcctcccgtgctggactcc
gacggctccttcttcctctacagcaagctcaccgtggacaagagcaggtggcagcaggggaacgtcttctcatgctccgtg
atgcatgaggctctgcacaaccactacacgcagaagagcctctccctgtctccgggtaaatgataa
(amino acids)
(SEQ ID NO: 57)
EVQLVESGGGLVKPGGSLRLSCAASGFTFSRYGMSWVRQAPGKRLEWVSTISGGGTYIYYPDSVKGRFTISRDNAKNPLYL
QMNSLRAEDTAVYYCPRDNYGRNYDYGMDYWGQGTLVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVDKKVEPKSCDKTHTCPPCPAPELLGGP
SVFLFPPKPKDTLMISRTPEVTCVVVDVSHEDPEVKFNWYVDGVEVHNAKTKPREEQYNSTYRVVSVLTVLHQDWLNGKEY
KCKVSNKALPAPIEKTISKAKGQPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK**
Human IgG1 heavy chain constant region sequence:
(DNA)
(SEQ ID NO: 58)
gctagcaccaagggcccatcggtcttccccctggcaccctcctccaagagcacctctgggggcacagcggccctgggctgc
ctggtcaaggactacttccccgaaccggtgacggtgtcgtggaactcaggcgccctgaccagcggcgtgcacaccttcccg
gctgtcctacagtcctcaggactctactccctcagcagcgtggtgacagtgccctccagcagcttgggcacccagacctac
atctgcaacgtgaatcacaagcccagcaacaccaaggtggacaagaaagttgagcccaaatcttgtgacaaaactcacaca
tgcccaccgtgcccagcacctgaactcctggggggaccgtcagtcttcctcttccccccaaaacccaaggacaccctcatg
atctcccggacccctgaggtcacatgcgtggtggtggacgtgagccacgaagaccctgaggtcaagttcaactggtacgtg
gacggcgtggaggtgcataatgccaagacaaagccgcgggaggagcagtacaacagcacgtaccgtgtggtcagcgtcctc
accgtcctgcaccaggactggctgaatggcaaggagtacaagtgcaaggtctccaacaaagccctcccagcccccatcgag
aaaaccatctccaaagccaaagggcagccccgagaaccacaggtgtacaccctgcccccatcccgggaggagatgaccaag
aaccaggtcagcctgacctgcctggtcaaaggcttctatcccagcgacatcgccgtggagtgggagagcaatgggcagccg
gagaacaactacaagaccacgcctcccgtgctggactccgacggctccttcttcctctacagcaagctcaccgtggacaag
agcaggtggcagcaggggaacgtcttctcatgctccgtgatgcatgaggctctgcacaaccactacacgcagaagagcctc
tccctgtctccgggtaaatgataa
(amino acids)
(SEQ ID NO: 59)
ASTKGPSVFPLAPSSKSTSGGTAALGCLVKDYFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKSRWQQGNVFSCSVMHEALHNHYTQKSL
SLSPGK**
Human IgG1 heavy chain constant region gBLOCK#1 sequence:
(DNA)
(SEQ ID NO: 60)
atggcatggattattggggccagggcaccctggtgaccgtgagcagcgctagcaccaagggcccatcggtcttccccctgg
caccctcctccaagagcacctctgggggcacagcggccctgggctgcctggtcaaggactacttccccgaaccggtgacgg
tgtcgtggaactcaggcgccctgaccagcggcgtgcacaccttcccggctgtcctacagtcctcaggactctactccctca
gcagcgtggtgacagtgccctccagcagcttgggcacccagacctacatctgcaacgtgaatcacaagcccagcaacacca
aggtggacaagaaagttgagcccaaatcttgtgacaaaactcacacatgcccaccgtgcccagcacctgaactcctggggg
gaccgtcagtcttcctcttccccccaaaacccaaggacaccctcatgatctcccggacccctgaggtcacatgcgtggtgg
tggacgtgagccacgaagaccctgaggtcaagttcaactggtacgtggacggcgtggaggtgcataatgccaag
Human IgG1 heavy chain constant region gBLOCK#2 sequence:
(DNA)
(SEQ ID NO: 61)
tacgtggacggcgtggaggtgcataatgccaagacaaagccgcgggaggagcagtacaacagcacgtaccgtgtggtcagc
gtcctcaccgtcctgcaccaggactggctgaatggcaaggagtacaagtgcaaggtctccaacaaagccctcccagccccc
atcgagaaaaccatctccaaagccaaagggcagccccgagaaccacaggtgtacaccctgcccccatcccgggaggagatg
accaagaaccaggtcagcctgacctgcctggtcaaaggcttctatcccagcgacatcgccgtggagtgggagagcaatggg
cagccggagaacaactacaagaccacgcctcccgtgctggactccgacggctccttcttcctctacagcaagctcaccgtg
gacaagagcaggtggcagcaggggaacgtcttctcatgctccgtgatgcatgaggctctgcacaaccactacacgcagaag
agcctctccctgtctccgggtaaatgataagtttaaacccgctgatcagcctcgactgtgccttctagttg
E6 heavy chain variable region overlapping sequence:
(DNA)
(SEQ ID NO: 62)
atggcatggattattggggccagggcaccct
IgG1 heavy chain constant region overlapping region sequence:
(DNA)
(SEQ ID NO: 63)
tacgtggacggcgtggaggtgcataatgccaag
pCDNA3.1 V5 and pSECTag2 overlapping sequence:
(DNA)
(SEQ ID NO: 64)
ccgctgatcagcctcgactgtgccttctagttg
Mouse E6 Light Chain variable region sequence:
(DNA)
(SEQ ID NO: 65)
caaattgttctcacccagtctccagcaatcatgtctgcatctccaggggaggaggtcaccctaacctgcagtgccacctca
agtgtaagttacatacactggttccagcagaggccaggcacttctcccaaactctggatttatagcacatccaacctggct
tctggagtccctgttcgcttcagtggcagtggatatgggacctcttactctctcacaatcagccgaatggaggctgaagat
gctgccacttattactgccagcaaaggagtagttccccattcacgttcggctcggggacaaagttggaaataaaa
(amino acids)
(SEQ ID NO: 66)
QIVLTQSPAIMSASPGEEVTLTCSATSSVSYIHWFQQRPGTSPKLWIYSTSNLASGVPVRFSGSGYGTSYSLTISRMEAED
AATYYCQQRSSSPFTFGSGTKLEIK
Mouse E6 light chain variable framework region 1 (FWR1) sequence:
(DNA)
(SEQ ID NO: 67)
caaattgttctcacccagtctccagcaatcatgtctgcatctccaggggaggaggtcaccctaacctgc
(amino acids)
(SEQ ID NO: 68)
QIVLTQSPAIMSASPGEEVTLTC
Mouse E6 light chain variable complementarity determining regions 1 (CDR1)
sequence:
(DNA)
(SEQ ID NO: 69)
AGTGCCACCTCAAGTGTAAGTTACATACAC
(amino acids)
(SEQ ID NO: 70)
SATSSVSYIH
Mouse E6 light chain variable framework region 2 (FWR2) sequence:
(DNA)
(SEQ ID NO: 71)
tggttccagcagaggccaggcacttctcccaaactctggatttat
(amino acids)
(SEQ ID NO: 72)
WFQQRPGTSPKLWIY
Mouse E6 light chain variable complementarity determining regions 2 (CDR2)
sequence:
(DNA)
(SEQ ID NO: 73)
agcacatccaacctggcttct
(amino acids)
(SEQ ID NO: 74)
STSNLAS
Mouse E6 light chain variable framework region 3 (FWR3) sequence:
(DNA)
(SEQ ID NO: 75)
ggagtccctgttcgcttcagtggcagtggatatgggacctcttactctctcacaatcagccgaatggaggctgaagatgc
tgccacttattactgc
(amino acids)
(SEQ ID NO: 76)
GVPVRFSGSGYGTSYSLTISRMEAEDAATYYC
Mouse E6 light chain variable complementarity determining regions 3 (CDR3)
sequence:
(DNA)
(SEQ ID NO: 77)
cagcaaaggagtagttccccattcacg
(amino acids)
(SEQ ID NO: 78)
QQRSSSPFT
IGKV3-11*02 light chain variable region sequence:
(DNA)
(SEQ ID NO: 79)
gaaattgtgttgacacagtctccagccaccctgtctttgtctccaggggaaagagccaccctctcctgcagggccagtca
gagtgttagcagctacttagcctggtaccaacagaaacctggccaggctcccaggctcctcatctatgatgcatccaaca
gggccactggcatcccagccaggttcagtggcagtgggtctgggagagacttcactctcaccatcagcagcctagagcct
gaagattttgcagtttattactgtcagcagcgtagcaactggcctcc
(amino acids)
(SEQ ID NO: 80)
EIVLTQSPATLSLSPGERATLSCRASQSVSSYLAWYQQKPGQAPRLLIYDASNRATGIPA
RFSGSGSGRDFTLTISSLEPEDFAVYYCQQRSNWPP
IGKV3-11*02 light chain variable framework region 1 (FWR1) acid sequence:
(DNA)
(SEQ ID NO: 81)
gaaattgtgttgacacagtctccagccaccctgtctttgtctccaggggaaagagccaccctctcctgc
(amino acids)
(SEQ ID NO: 82)
EIVLTQSPATLSLSPGERATLSC
IGKV3-11*02 light chain variable complementarity determining regions 1 (CDR1)
sequence:
(DNA)
(SEQ ID NO: 83)
agggccagtcagagtgttagcagctacttagcc
(amino acids)
(SEQ ID NO: 84)
RASQSVSSYLA
IGKV3-11*02 light chain variable framework region 2 (FWR2) sequence:
(DNA)
(SEQ ID NO: 85)
tggtaccaacagaaacctggccaggctcccaggctcctcatctat
(amino acids)
(SEQ ID NO 86)
WYQQKPGQAPRLLIY
IGKV3-11*02 light chain variable complementarity determining regions 2 (CDR2)
sequence:
(DNA)
(SEQ ID NO: 87)
gatgcatccaacagggccact
(amino acids)
(SEQ ID NO: 88)
DASNRAT
IGKV3-11*02 light chain variable framework region 3 (FWR3) sequence:
(DNA)
(SEQ ID NO: 89)
ggcatcccagccaggttcagtggcagtgggtctgggagagacttcactctcaccatcagcagcctagagcctgaagattt
tgcagtttattactgt
(amino acids)
(SEQ ID NO: 90)
GIPARFSGSGSGTDFTLTISSLEPEDFAVYYC
IGKV3-11*02 light chain variable complementarity determining regions3 (CDR3)
sequence:
(DNA)
(SEQ ID NO: 91)
cagcagcgtagcaactggcctcc
(amino acids)
(SEQ ID NO: 92)
QQRSNWPP
Humanized E6 light chain variable region sequence:
(DNA)
(SEQ ID NO: 93)
gaaattgtgttgacacagtctccagccaccctgtctttgtctccaggggaaagagccaccctcacctgcagcgccaccagc
agtgttagctacatccactggtaccaacagaggcctggccagagccccaggctcctcatctatagcacctccaacctggcc
agcggcatcccagccaggttcagtggcagtgggtctgggagcgactacactctcaccatcagcagcctagagcctgaagat
tttgcagtttattactgtcagcagcgtagcagctcccctttcacctttggcagcggcaccaaagtggaaattaaa
(amino acids)
(SEQ ID NO: 94)
EIVLTQSPATLSLSPGERATLTCSATSSVSYIHWYQQRPGQSPRLLIYSTSNLASGIPARFSGSGSGSDYTLTISSLEPED
FAVYYCQQRSSSPFTFGSGTKVEIK
Humanized E6 light chain variable framework region 1 (FWR1) acid sequence:
(DNA)
(SEQ ID NO: 95)
gaaattgtgttgacacagtctccagccaccctgtctttgtctccaggggaaagagccaccctcacctgc
(amino acids)
(SEQ ID NO: 96)
EIVLTQSPATLSLSPGERATLTC
Humanized E6 light chain variable complementarity determining regions 1 (CDR1)
sequence:
(DNA)
(SEQ ID NO: 97)
agcgccaccagcagtgttagctacatccac
(amino acids)
(SEQ ID NO: 98)
SATSSVSYIH
Humanized E6 heavy light variable framework region 2 (FWR2) acid sequence:
(DNA)
(SEQ ID NO: 99)
tggtaccaacagaggcctggccagagccccaggctcctcatctat
(amino acids)
(SEQ ID NO: 100)
WYQQRPGQSPRLLIY
Humanized E6 light chain variable complementarity determining regions 2 (CDR2)
sequence:
(DNA)
(SEQ ID NO: 101)
agcacctccaacctggccagc
(amino acids)
(SEQ ID NO: 102)
STSNLAS
Humanized E6 light chain variable framework region 3 (FWR3) acid sequence:
(DNA)
(SEQ ID NO: 103)
ggcatcccagccaggttcagtggcagtgggtctgggagcgactacactctcaccatcagcagcctagagcctgaagatttt
gcagtttattactgt
(amino acids)
(SEQ ID NO: 104)
GIPARFSGSGSGSDYTLTISSLEPEDFAVYYC
Humanized E6 light chain variable complementarity determining regions 3 (CDR3)
sequence:
(DNA)
(SEQ ID NO: 105)
cagcagcgtagcagctcccctttcacc
(amino acids)
(SEQ ID NO: 106)
QQRSSSPFT
Humanized E6 Kappa light chain synthesized by Genescript:
(DNA)
(SEQ ID NO: 107)
gaattctaagcttgggccaccatggaagccccagcgcagcttctcttcctcctgctactctggctcccagataccactgga
gaaattgtgttgacacagtctccagccaccctgtctttgtctccaggggaaagagccaccctcacctgcagcgccaccagc
agtgttagctacatccactggtaccaacagaggcctggccagagccccaggctcctcatctatagcacctccaacctggcc
agcggcatcccagccaggttcagtggcagtgggtctgggagcgactacactctcaccatcagcagcctagagcctgaagat
tttgcagtttattactgtcagcagcgtagcagctcccctttcacctttggcagcggcaccaaagtggaaattaaaaggacg
gtggctgcaccatctgtcttcatcttcccgccatctgatgagcagttgaaatctggaactgcctctgttgtgtgcctgctg
aataacttctatcccagagaggccaaagtacagtggaaggtggataacgccctccaatcgggtaactcccaggagagtgtc
acagagcaggacagcaaggacagcacctacagcctcagcagcaccctgacgctgagcaaagcagactacgagaaacacaaa
gtctacgcctgcgaagtcacccatcagggcctgagctcgcccgtcacaaagagcttcaacaggggagagtgttagtaagtt
taaactctaga
(amino acids)
(SEQ ID NO: 108)
EF*AWATMEAPAQLLFLLLLWLPDTTGEIVLTQSPATLSLSPGERATLTCSATSSVSYIHWYQQRPGQSPRLLIYSTSNLA
SGIPARFSGSGSGSDYTLTISSLEPEDFAVYYCQQRSSSPFTFGSGTKVEIKRTVAAPSVFIFPPSDEQLKSGTASVVCLL
NNFYPREAKVQWKVDNALQSGNSQESVTEQDSKDSTYSLSSTLTLSKADYEKHKVYACEVTHQGLSSPVTKSFNRGEC**V
*TLX
Human Kappa light chain constant region sequence:
(DNA)
(SEQ ID NO: 109)
aggacggtggctgcaccatctgtcttcatcttcccgccatctgatgagcagttgaaatctggaactgcctctgttgtgtgc
ctgctgaataacttctatcccagagaggccaaagtacagtggaaggtggataacgccctccaatcgggtaactcccaggag
agtgtcacagagcaggacagcaaggacagcacctacagcctcagcagcaccctgacgctgagcaaagcagactacgagaaa
cacaaagtctacgcctgcgaagtcacccatcagggcctgagctcgcccgtcacaaagagcttcaacaggggagagtgttag
(amino acids)
(SEQ ID NO: 110)
RTVAAPSVFIFPPSDEQLKSGTASVVCLLNNFYPREAKVQWKVDNALQSGNSQESVTEQDSKDSTYSLSSTLTLSKADYEK
HKVYACEVTHQGLSSPVTKSFNRGEC
Humanized E6 lambda light chain sequence:
(DNA)
(SEQ ID NO: 111)
gaaattgtgttgacacagtctccagccaccctgtctttgtctccaggggaaagagccaccctcacctgcagcgccaccagc
agtgttagctacatccactggtaccaacagaggcctggccagagccccaggctcctcatctatagcacctccaacctggcc
agcggcatcccagccaggttcagtggcagtgggtctgggagcgactacactctcaccatcagcagcctagagcctgaagat
tttgcagtttattactgtcagcagcgtagcagctcccctttcacctttggcagcggcaccaaagtggaaattaaaggtcag
cccaaggctgccccctcggtcactctgttcccgccctcctctgaggagcttcaagccaacaaggccacactggtgtgtctc
ataagtgacttctacccgggagccgtgacagtggcctggaaggcagatagcagccccgtcaaggcgggagtggagaccacc
acaccctccaaacaaagcaacaacaagtacgcggccagcagctatctgagcctgacgcctgagcagtggaagtcccacaga
agctacagctgccaggtcacgcatgaagggagcaccgtggagaagacagtggcccctacagaatgttcatagtaa
(amino acids)
(SEQ ID NO: 112)
EIVLTQSPATLSLSPGERATLTCSATSSVSYIHWYQQRPGQSPRLLIYSTSNLASGIPARFSGSGSGSDYTLTISSLEPED
FAVYYCQQRSSSPFTFGSGTKVEIKGQPKAAPSVTLFPPSSEELQANKATLVCLISDFYPGAVTVAWKADSSPVKAGVETT
TPSKQSNNKYAASSYLSLTPEQWKSHRSYSCQVTHEGSTVEKTVAPTECS**
Humanized lambda light chain constant region sequence:
(DNA)
(SEQ ID NO: 113)
ggtcagcccaaggctgccccctcggtcactctgttcccgccctcctctgaggagcttcaagccaacaaggccacactggtg
tgtctcataagtgacttctacccgggagccgtgacagtggcctggaaggcagatagcagccccgtcaaggcgggagtggag
accaccacaccctccaaacaaagcaacaacaagtacgcggccagcagctatctgagcctgacgcctgagcagtggaagtcc
cacagaagctacagctgccaggtcacgcatgaagggagcaccgtggagaagacagtggcccctacagaatgttcatagtaa
(amino acids)
(SEQ ID NO: 114)
GQPKAAPSVTLFPPSSEELQANKATLVCLISDFYPGAVTVAWKADSSPVKAGVETTTPSKQSNNKYAASSYLSLTPEQWKS
HRSYSCQVTHEGSTVEKTVAPTECS**
Human lambda light chain constant region gBLOCK#3 sequence:
(DNA)
(SEQ ID NO: 115)
agcgccaccagcagtgttagctacatccactggtaccaacagaggcctggccagagccccaggctcctcatctatagcacc
tccaacctggccagcggcatcccagccaggttcagtggcagtgggtctgggagcgactacactctcaccatcagcagccta
gagcctgaagattttgcagtttattactgtcagcagcgtagcagctcccctttcacctttggcagcggcaccaaagtggaa
attaaaggtcagcccaaggctgccccctcggtcactctgttcccgccctcctctgaggagcttcaagccaacaaggccaca
ctggtgtgtctcataagtgacttctacccgggagccgtgacagtggcctggaaggcagatagcagccccgtcaaggcggga
gtggagaccaccacaccctccaaacaaagcaacaacaagtacgcggccagcagctatctgagcctgacgcctgagcagtgg
aagtcccacagaagctacagctgccaggtcacgcatgaagggagcaccgtggagaagacagtggcccctacagaatgttca
tagtaagtttaaacccgctgatcagcctcgactgtgccttctagttg
E6 light chain variable region overlapping sequence:
(DNA)
(SEQ ID NO: 116)
agcgccaccagcagtgttagctacatccact
pCDNA3.1 V5 and pSECTag2 overlapping sequence:
(DNA)
(SEQ ID NO: 117)
ccgctgatcagcctcgactgtgccttctagttg
Mouse C2 heavy chain variable region sequence:
(DNA)
(SEQ ID NO: 118)
gaggtccagctggaggagtcagggggaggcttagtgaagcctggagggtccctgaaactctcctgtgcagcctctggatt
cactttcagtggctatgccatgtcttgggttcgccagactccggagaagaggctggagtgggtcgcaaccattagtagtg
gtggtacttatatctactatccagacagtgtgaaggggcgattcaccatctccagagacaatgccaagaacaccctgtac
ctgcaaatgagcagtctgaggtctgaggacacggccatgtattactgtgcaagacttgggggggataattactacgaata
cttcgatgtctggggcgcagggaccacggtcaccgtctcctccgccaaaacgacacccccatctgtctat
(amino acids)
(SEQ ID NO: 119)
EVQLEESGGGLVKPGGSLKLSCAASGFTFSGYAMSWVRQTPEKRLEWVATISSGGTYIYYPDSVKGRFTISRDNAKNTLY
LQMSSLRSEDTAMYYCARLGGDNYYEYFDVWGAGTTVTVSSAKTTPPSVY
Mouse C2 heavy chain variable framework region 1 (FWR1) sequence:
(DNA)
(SEQ ID NO: 120)
gaggtccagctggaggagtcagggggaggcttagtgaagcctggagggtccctgaaactctcctgtgcagcctctggatt
cactttcagt
(amino acids)
(SEQ ID NO: 121)
EVQLEESGGGLVKPGGSLKLSCAASGFTFS
Mouse C2 heavy chain variable complementarity determining regions 1 (CDR1)
sequence:
(DNA)
(SEQ ID NO: 122)
ggctatgccatgtct
(amino acids)
(SEQ ID NO: 123)
GYAMS
Mouse C2 heavy chain variable framework region 2 (FWR2) sequence:
(DNA)
(SEQ ID NO: 124)
tgggttcgccagactccggagaagaggctggagtgggtcgca
(amino acids)
(SEQ ID NO: 125)
WVRQTPEKRLEWVA
Mouse C2 heavy chain variable complementarity determining regions 2 (CDR2)
sequence:
(DNA)
(SEQ ID NO: 126)
accattagtagtggtggtacttatatctactatccagacagtgtgaagggg
(amino acids)
(SEQ ID NO: 127)
TISSGGTYIYYPDSVKG
Mouse C2 heavy chain variable framework region 3 (FWR3) sequence:
(DNA)
(SEQ ID NO: 128)
cgattcaccatctccagagacaatgccaagaacaccctgtacctgcaaatgagcagtctgaggtctgaggacacggccatg
tattactgtgcaaga
(amino acids)
(SEQ ID NO: 129)
RFTISRDNAKNTLYLQMSSLRSEDTAMYYCAR
Mouse C2 heavy chain variable complementarity determining regions 3 (CDR3)
sequence:
(DNA)
(SEQ ID NO: 130)
cttgggggggataattactacgaatacttcgatgtc
(amino acids)
(SEQ ID NO: 131)
LGGDNYYEYFDV
IGHV3-21*04 heavy chain variable region sequence:
(DNA)
(SEQ ID NO: 132)
gaggtgcagctggtggagtctgggggaggcctggtcaagcctggggggtccctgagactctcctgtgcagcctctggatt
caccttcagtagctatagcatgaactgggtccgccaggctccagggaaggggctggagtgggtctcatccattagtagta
gtagtagttacatatactacgcagactcagtgaagggccgattcaccatctccagagacaacgccaagaactcactgtat
ctgcaaatgaacagcctgagagccgaggacacggccgtgtattactgtgcgaga
(amino acids)
(SEQ ID NO: 133)
EVQLVESGGGLVKPGGSLRLSCAASGFTFSSYSMNWVRQAPGKGLEWVSSISSSSSYIYYADSVKGRFTISRDNAKNSLY
LQMNSLRAEDTAVYYCAR
IGHV3-21*04 heavy chain variable framework region 1 (FWR1) sequence:
(DNA)
(SEQ ID NO: 134)
gaggtgcagctggtggagtctgggggaggcctggtcaagcctggggggtccctgagactctcctgtgcagcctctggatt
caccttcagt
(amino acids)
(SEQ ID NO: 135)
EVQLVESGGGLVKPGGSLRLSCAASGFTFS
IGHV3-21*04 heavy chain variable complementarity determining regions 1 (CDR1)
sequence:
(DNA)
(SEQ ID NO: 136)
agctatagcatgaac
(amino acids)
(SEQ ID NO: 137)
SYSMN
IGHV3-21*04 heavy chain variable framework region 2 (FWR2) sequence:
(DNA)
(SEQ ID NO: 138)
gggtccgccaggctccagggaaggggctggagtgggtctca
(amino acids)
(SEQ ID NO: 139)
WVRQAPGKGLEWVS
IGHV3-21*04 heavy chain variable complementarity determining regions 2 (CDR2)
sequence:
(DNA)
(SEQ ID NO: 140)
tccattagtagtagtagtagttacatatactacgcagactcagtgaagggc
(amino acids)
(SEQ ID NO: 141)
SISSSSSYIYYADSVKG
IGHV3-21*04 heavy chain variable framework region 3 (FWR3) sequence:
(DNA)
(SEQ ID NO: 142)
cgattcaccatctccagagacaacgccaagaactcactgtatctgcaaatgaacagcctgagagccgaggacacggccgt
gtattactgtgcgaga
(amino acids)
(SEQ ID NO: 143)
RFTISRDNAKNSLYLQMNSLRAEDTAVYYCAR
Humanized C2 heavy chain variable region sequence:
(DNA)
(SEQ ID NO: 144)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctcc
(amino acids)
(SEQ ID NO: 145)
EVQLVESGGGLVKPGGSLRLSCAASGFTFSGYAMSWVRQAPGKGLEWVSTISSGGTYIYYPDSVKGRFTISRDNAKNSLYL
QMNSLRAEDTAVYYCARLGGDNYYEYFDVWGKGTTVTVSS
Humanized C2 heavy chain variable framework region 1 (FWR1) sequence:
(DNA)
(SEQ ID NO: 146)
gaggtgcagctggtggagtctgggggaggcctggtcaagcctggggggtccctgagactctcctgtgcagcctctggattc
accttcagt
(amino acids)
(SEQ ID NO: 147)
EVQLVESGGGLVKPGGSLRLSCAASGFTFS
Humanized C2 heavy chain variable complementarity determining regions 1 (CDR1)
sequence:
(DNA)
(SEQ ID NO: 148)
ggctatgccatgagc
(amino acids)
(SEQ ID NO: 149)
GYAMS
Humanized C2 heavy chain variable framework region 2 (FWR2) sequence:
(DNA)
(SEQ ID NO: 150)
tgggtccgccaggctccagggaaggggctggagtgggtctcaa
(amino acids)
(SEQ ID NO: 151)
WVRQAPGKGLEWVS
Humanized C2 heavy chain variable complementarity determining regions 2 (CDR2)
sequence:
(DNA)
(SEQ ID NO: 152)
accattagtagtggcggaacctacatatactaccccgactcagtgaagggc
(amino acids)
(SEQ ID NO: 153)
TISSGGTYIYYPDSVKG
Humanized C2 heavy chain variable framework region 3 (FWR3) sequence:
(DNA)
(SEQ ID NO: 154)
cgattcaccatctccagagacaacgccaagaactcactgtatctgcaaatgaacagcctgagagccgaggacacggccgtg
tattactgtgcgaga
(amino acids)
(SEQ ID NO: 155)
RFTISRDNAKNSLYLQMNSLRAEDTAVYYCAR
Humanized C2 heavy chain variable complementarity determining regions 3 (CDR3)
sequence:
(DNA)
(SEQ ID NO: 156)
cttgggggggataattactacgaatacttcgatgtc
(amino acids)
(SEQ ID NO: 157)
LGGDNYYEYFDV
Humanized C2 IgG1 heavy chain sequence
(DNA)
(SEQ ID NO: 157)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccgctagcaccaagggcccatcggtcttccccctggcaccctcc
tccaagagcacctctgggggcacagcggccctgggctgcctggtcaaggactacttccccgaaccggtgacggtgtcgtgg
aactcaggcgccctgaccagcggcgtgcacaccttcccggctgtcctacagtcctcaggactctactccctcagcagcgtg
gtgacagtgccctccagcagcttgggcacccagacctacatctgcaacgtgaatcacaagcccagcaacaccaaggtggac
aagaaagttgagcccaaatcttgtgacaaaactcacacatgcccaccgtgcccagcacctgaactcctggggggaccgtca
gtcttcctcttccccccaaaacccaaggacaccctcatgatctcccggacccctgaggtcacatgcgtggtggtggacgtg
agccacgaagaccctgaggtcaagttcaactggtacgtggacggcgtggaggtgcataatgccaagacaaagccgcgggag
gagcagtacaacagcacgtaccgtgtggtcagcgtcctcaccgtcctgcaccaggactggctgaatggcaaggagtacaag
tgcaaggtctccaacaaagccctcccagcccccatcgagaaaaccatctccaaagccaaagggcagccccgagaaccacag
gtgtacaccctgcccccatcccgggaggagatgaccaagaaccaggtcagcctgacctgcctggtcaaaggcttctatccc
agcgacatcgccgtggagtgggagagcaatgggcagccggagaacaactacaagaccacgcctcccgtgctggactccgac
ggctccttcttcctctacagcaagctcaccgtggacaagagcaggtggcagcaggggaacgtcttctcatgctccgtgatg
catgaggctctgcacaaccactacacgcagaagagcctctccctgtctccgggtaaatgataa
(amino acids)
(SEQ ID NO: 158)
EVQLVESGGGLVKPGGSLRLSCAASGFTFSGYAMSWVRQAPGKGLEWVSTISSGGTYIYYPDSVKGRFTISRDNAKNSLYL
QMNSLRAEDTAVYYCARLGGDNYYEYFDVWGKGTTVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK**
Humanized C2 gBLOCK#4 sequence:
(DNA)
(SEQ ID NO: 160)
actcactatagggagacccaagctggctagttaagcttgggccaccatggagacagacacactcctgctatgggtactgct
gctctgggttccaggttccactggtgacgaggtgcagctggtggagtctgggggaggcctggtcaagcctggggggtccct
gagactctcctgtgcagcctctggattcaccttcagtggctatgccatgagctgggtccgccaggctccagggaaggggct
ggagtgggtctcaaccattagtagtggcggaacctacatatactaccccgactcagtgaagggccgattcaccatctccag
agacaacgccaagaactcactgtatctgcaaatgaacagcctgagagccgaggacacggccgtgtattactgtgcgagact
tgggggggataattactacgaatacttcgatgtctggggcaaagggaccacggtcaccgtctcctccgctagcaccaaggg
cccatcggtcttccccctggcaccctcctccaagagcacctctgggggcacagcggccctgggctgcctggtcaaggacta
cttccccgaaccggtgacggtgtcgtggaactcaggcgccctgaccagc
pCDNA3.1 V5 overlapping sequence:
(DNA)
(SEQ ID NO: 161)
actcactatagggagacccaagctggctagtt
Human IgG1 constant region overlapping sequence:
(DNA)
(SEQ ID NO: 162)
gacggtgtcgtggaactcaggcgccctgaccagc
Humanized C2 IgG2 heavy chain sequence
(DNA)
(SEQ ID NO: 163)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccgcctccaccaagggcccatcggtcttccccctggcgccctgc
tccaggagcacctccgagagcacagccgccctgggctgcctggtcaaggactacttccccgaaccggtgacggtgtcgtgg
aactcaggcgctctgaccagcggcgtgcacaccttcccagctgtcctacagtcctcaggactctactccctcagcagcgtg
gtgaccgtgccctccagcaacttcggcacccagacctacacctgcaacgtagatcacaagcccagcaacaccaaggtggac
aagacagttgagcgcaaatgttgtgtcgagtgcccaccgtgcccagcaccacctgtggcaggaccgtcagtcttcctcttc
cccccaaaacccaaggacaccctcatgatctcccggacccctgaggtcacgtgcgtggtggtggacgtgagccacgaagac
cccgaggtccagttcaactggtacgtggacggcgtggaggtgcataatgccaagacaaagccacgggaggagcagttcaac
agcacgttccgtgtggtcagcgtcctcaccgttgtgcaccaggactggctgaacggcaaggagtacaagtgcaaggtctcc
aacaaaggcctcccagcccccatcgagaaaaccatctccaaaaccaaagggcagccccgagaaccacaggtgtacaccctg
cccccatcccgggaggagatgaccaagaaccaggtcagcctgacctgcctggtcaaaggcttctaccccagcgacatcgcc
gtggagtgggagagcaatgggcagccggagaacaactacaagaccacacctcccatgctggactccgacggctccttcttc
ctctacagcaagctcaccgtggacaagagcaggtggcagcaggggaacgtcttctcatgctccgtgatgcatgaggctctg
cacaaccactacacgcagaagagcctctccctgtctccgggtaaatagtaa
(amino acids)
(SEQ ID NO: 164)
EVQLVESGGGLVKPGGSLRLSCAASGFTFSGYAMSWVRQAPGKGLEWVSTISSGGTYIYYPDSVKGRFTISRDNAKNSLYL
QMNSLRAEDTAVYYCARLGGDNYYEYFDVWGKGTTVTVSSASTKGPSVFPLAPCSRSTSESTAALGCLVKDYFPEPVTVSW
NSGALTSGVHTFPAVLQSSGLYSLSSVVTVPSSNFGTQTYTCNVDHKPSNTKVDKTVERKCCVECPPCPAPPVAGPSVFLF
PPKPKDTLMISRTPEVTCVVVDVSHEDPEVQFNWYVDGVEVHNAKTKPREEQFNSTFRVVSVLTVVHQDWLNGKEYKCKVS
NKGLPAPIEKTISKTKGQPREPQVYTLPPSREEMTKNQVSLTCLVKGFYPSDIAVEWESNGQPENNYKTTPPMLDSDGSFF
LYSKLTVDKSRWQQGNVFSCSVMHEALHNHYTQKSLSLSPGK**
Humanized C2 gBLOCK#5 sequence:
(DNA)
(SEQ ID NO: 165)
tgctctgggttccaggttccactggtgacgcggcccagccggccgaggtgcagctggtggagtctgggggaggcctggtca
agcctggggggtccctgagactctcctgtgcagcctctggattcaccttcagtggctatgccatgagctgggtccgccagg
ctccagggaaggggctggagtgggtctcaaccattagtagtggcggaacctacatatactaccccgactcagtgaagggcc
gattcaccatctccagagacaacgccaagaactcactgtatctgcaaatgaacagcctgagagccgaggacacggccgtgt
attactgtgcgagacttgggggggataattactacgaatacttcgatgtctggggcaaagggaccacggtcaccgtctcct
ccgcctccaccaagggcccatcggtcttccccctggcgccctgctccaggagcacctccgagagcacagccgccctgggct
gcctggtcaaggactacttccccgaaccggtgacggtgtcgtggaactcaggcgctctgacca
pSEC Tag2 overlapping sequence:
(DNA)
(SEQ ID NO: 166)
tgctctgggttccaggttccactggtgacgc
Human IgG2 constant region overlapping sequence:
(DNA)
(SEQ ID NO: 167)
gacggtgtcgtggaactcaggcgctctgacca
Mouse C2 light chain variable region sequence:
(DNA)
(SEQ ID NO: 168)
gacattgtgatcacacagtctacagcttccttaggtgtatctctggggcagagggccaccatctcatgcagggccagcaaa
agtgtcagtacatctggctatagttatatgcactggtaccaacagagaccaggacagccacccaaactcctcatctatctt
gcatccaacctagaatctggggtccctgccaggttcagtggcagtgggtctgggacagacttcaccctcaacatccatcct
gtggaggaggaggatgctgcaacctattactgtcagcacagtagggagcttccgttcacgttcggaggggggaccaagctg
gagataaaacgggctgatgctgcaccaactgtatcc
(amino acids)
(SEQ ID NO: 169)
DIVITQSTASLGVSLGQRATISCRASKSVSTSGYSYMHWYQQRPGQPPKLLIYLASNLESGVPARFSGSGSGTDFTLNIHP
VEEEDAATYYCQHSRELPFTFGGGTKLEIKRADAAPTVS
Mouse C2 light chain variable framework region 1 (FWR1) sequence:
(DNA)
(SEQ ID NO: 170)
gacattgtgatcacacagtctacagcttccttaggtgtatctctggggcagagggccaccatctcatgc
(amino acids)
(SEQ ID NO: 171)
DIVITQSTASLGVSLGQRATISC
Mouse C2 light chain variable complementarity determining regions 1 (CDR1)
sequence:
(DNA)
(SEQ ID NO: 172)
agggccagcaaaagtgtcagtacatctggctatagttatatgcac
(amino acids)
(SEQ ID NO: 173)
RASKSVSTSGYSYMH
Mouse C2 light chain variable framework region 2 (FWR2) sequence:
(DNA)
(SEQ ID NO: 174)
tggtaccaacagagaccaggacagccacccaaactcctcatctat
(amino acids)
(SEQ ID NO: 175)
WYQQRPGQPPKLLIY
Mouse C2 light chain variable complementarity determining regions 2 (CDR2)
sequence:
(DNA)
(SEQ ID NO: 176)
cttgcatccaacctagaatc
(amino acids)
(SEQ ID NO: 177)
LASNLES
Mouse C2 light chain variable framework region 3 (FWR3) sequence:
(DNA)
(SEQ ID NO: 178)
tggggtccctgccaggttcagtggcagtgggtctgggacagacttcaccctcaacatccatcctgtggaggaggaggatgc
tgcaacctattactgt
(amino acids)
(SEQ ID NO: 179)
GVPARFSGSGSGTDFTLNIHPVEEEDAATYYC
Mouse C2 light chain variable complementarity determining regions 3 (CDR3)
sequence:
(DNA)
(SEQ ID NO: 180)
cagcacagtagggagcttccgttcacg
(amino acids)
(SEQ ID NO: 181)
QHSRELPFT
IGKV7-3*01 light chain variable region sequence:
(DNA)
(SEQ ID NO: 182)
gacattgtgctgacccagtctccagcctccttggccgtgtctccaggacagagggccaccatcacctgcagagccagtgag
agtgtcagtttcttgggaataaacttaattcactggtatcagcagaaaccaggacaacctcctaaactcctgatttaccaa
gcatccaataaagacactggggtcccagccaggttcagcggcagtgggtctgggaccgatttcaccctcacaattaatcct
gtggaagctaatgatactgcaaattattactgtctgcagagtaagaattttcctcccaca
(amino acid)
(SEQ ID NO: 183)
DIVLTQSPASLAVSPGQRATITCRASESVSFLGINLIHWYQQKPGQPPKLLIYQASNKDTGVPARFSGSGSGTDFTLTINP
VEANDTANYYCLQSKNFPPT
IGKV7-3*01 light chain variable framework region 1 (FWR1) sequence:
(DNA)
(SEQ ID NO: 184)
gacattgtgctgacccagtctccagcctccttggccgtgtctccaggacagagggccaccatcacctgc
(amino acids)
(SEQ ID NO: 185)
DIVLTQSPASLAVSPGQRATITC
IGKV7-3*01 light chain variable complementarity determining regions 1 (CDR1)
sequence:
(DNA)
(SEQ ID NO: 186)
agagccagtgagagtgtcagtttcttgggaataaacttaattcac
(amino acids)
(SEQ ID NO: 187)
RASESVSFLGINLIH
IGKV7-3*01 light chain variable framework region 2 (FWR2) sequence:
(DNA)
(SEQ ID NO: 188)
tggtatcagcagaaaccaggacaacctcctaaactcctgatttac
(amino acids)
(SEQ ID NO: 189)
WYQQKPGQPPKLLIY
IGKV7-3*01 light chain variable complementarity determining regions 2 (CDR2)
sequence:
(DNA)
(SEQ ID NO: 190)
caagcatccaataaagacact
(amino acids)
(SEQ ID NO: 191)
QASNKDT
IGKV7-3*01 light chain variable framework region 3 (FWR3) sequence:
(DNA)
(SEQ ID NO: 192)
ggggtcccagccaggttcagcggcagtgggtctgggaccgatttcaccctcacaattaatcctgtggaagctaatgatact
gcaaattattactgt
(amino acids)
(SEQ ID NO: 193)
GVPARFSGSGSGTDFTLTINPVEANDTANYYC
Humanized C2 light chain variable region sequence:
(DNA)
(SEQ ID NO: 194)
gacattgtgctgacccagtctccagcctccttggccgtgtctccaggacagagggccaccatcacctgcagagccagtaag
agtgtcagtaccagcggatactcctacatgcactggtatcagcagaaaccaggacaacctcctaaactcctgatttacctg
gcatccaatctggagagcggggtcccagccaggttcagcggcagtgggtctgggaccgatttcaccctcacaattaatcct
gtggaagctaatgatactgcaaattattactgtcagcacagtagggagctgcctttcacattcggcggagggaccaaggtg
gagatcaaacgaact
(amino acids)
(SEQ ID NO: 195)
DIVLTQSPASLAVSPGQRATITCRASKSVSTSGYSYMHWYQQKPGQPPKLLIYLASNLESGVPARFSGSGSGTDFTLTINP
VEANDTANYYCQHSRELPFTFGGGTKVEIKRT
Humanized C2 light chain variable framework region 1 (FWR1) acid sequence:
(DNA)
(SEQ ID NO: 196)
gacattgtgctgacccagtctccagcctccttggccgtgtctccaggacagagggccaccatcacctgc
(amino acids)
(SEQ ID NO: 197)
DIVLTQSPASLAVSPGQRATITC
Humanized C2 light chain variable complementarity determining regions 1 (CDR1)
sequence:
(DNA)
(SEQ ID NO: 198)
agagccagtaagagtgtcagtaccagcggatactcctacatgcac
(amino acids)
(SEQ ID NO: 199)
RASKSVSTSGYSYMH
Humanized C2 heavy light variable framework region 2 (FWR2) acid sequence:
(DNA)
(SEQ ID NO: 200)
tggtatcagcagaaaccaggacaacctcctaaactcctgatttac
(amino acids)
(SEQ ID NO: 201)
WYQQKPGQPPKLLIY
Humanized C2 light chain variable complementarity determining regions 2 (CDR2)
sequence:
(DNA)
(SEQ ID NO: 202)
ctggcatccaatctggagagc
(amino acids)
(SEQ ID NO: 203)
LASNLES
Humanized C2 light chain variable framework region 3 (FWR3) acid sequence:
(DNA)
(SEQ ID NO: 204)
ggggtcccagccaggttcagcggcagtgggtctgggaccgatttcaccctcacaattaatcctgtggaagctaatgatact
gcaaattattactgt
(amino acids)
(SEQ ID NO: 205)
GVPARFSGSGSGTDFTLTINPVEANDTANYYC
Humanized C2 light chain variable complementarity determining regions 3 (CDR3)
sequence:
(DNA)
(SEQ ID NO: 206)
cagcacagtagggagctgcctttcaca
(amino acids)
(SEQ ID NO: 207)
QHSRELPFT
Humanized C2 light chain variable complementarity determining regions 3 (CDR3)
sequence:
(DNA)
(SEQ ID NO: 208)
ctgcagagtaagaattttcctcccaca
(amino acids)
(SEQ ID NO: 209)
LQSKNFPPT
Humanized C2 gBLOCK#6 sequence (Kappa light chain in pCDNA3.1 V5):
(DNA)
(SEQ ID NO: 210)
actcactatagggagacccaagctggctagttaagcttgggccaccatggagacagacacactcctgctatgggtactgct
gctctgggttccaggttccactggtgacgacattgtgctgacccagtctccagcctccttggccgtgtctccaggacagag
ggccaccatcacctgcagagccagtaagagtgtcagtaccagcggatactcctacatgcactggtatcagcagaaaccagg
acaacctcctaaactcctgatttacctggcatccaatctggagagcggggtcccagccaggttcagcggcagtgggtctgg
gaccgatttcaccctcacaattaatcctgtggaagctaatgatactgcaaattattactgtcagcacagtagggagctgcc
tttcacattcggcggagggaccaaggtggagatcaaacgaactacggtggctgcaccatctgtcttcatcttcccgccatc
tgatgagcagttgaaatctggaactgcctctgttgtgtgcctgctgaataacttctatcccagagaggccaaagtacagtg
gaaggtggataacgccctccaatcgggtaactcccaggagagtgtcacagagcaggacagcaaggacagcacctacagcct
cagcagcaccctgacgctgagcaaagcagactacgagaaacacaaagtctacgcctgcgaagtcacccatcagggcctgag
ctcgcccgtcacaaagagcttcaacaggggagagtgttagtaagtttaaacccgctgatcagcctcgactgtgccttctag
ttg
pCDNA3.1 V5 5′ overlapping sequence:
(DNA)
(SEQ ID NO: 211)
actcactatagggagacccaagctggctagtt
pCDNA3.1 V5 3′ overlapping sequence:
(DNA)
(SEQ ID NO: 212)
ccgctgatcagcctcgactgtgccttctagttg
Humanized C2 gBLOCK#7 sequence (Kappa light chain in pSEC Tag2):
(DNA)
(SEQ ID NO: 213)
tgctctgggttccaggttccactggtgacgcggcccagccggccgacattgtgctgacccagtctccagcctccttggccg
tgtctccaggacagagggccaccatcacctgcagagccagtaagagtgtcagtaccagcggatactcctacatgcactggt
atcagcagaaaccaggacaacctcctaaactcctgatttacctggcatccaatctggagagcggggtcccagccaggttca
gcggcagtgggtctgggaccgatttcaccctcacaattaatcctgtggaagctaatgatactgcaaattattactgtcagc
acagtagggagctgcctttcacattcggcggagggaccaaggtggagatcaaacgaactacggtggctgcaccatctgtct
tcatcttcccgccatctgatgagcagttgaaatctggaactgcctctgttgtgtgcctgctgaataacttctatcccagag
aggccaaagtacagtggaaggtggataacgccctccaatcgggtaactcccaggagagtgtcacagagcaggacagcaagg
acagcacctacagcctcagcagcaccctgacgctgagcaaagcagactacgagaaacacaaagtctacgcctgcgaagtca
cccatcagggcctgagctcgcccgtcacaaagagcttcaacaggggagagtgttagtaagtttaaacccgctgatcagcct
cgactgtgccttctagttg
pSEC Tag2 5′ overlapping sequence:
(DNA)
(SEQ ID NO: 214)
tgctctgggttccaggttccactggtgacgc
pSEC Tag2 3′ overlapping sequence:
(DNA)
(SEQ ID NO: 215)
ccgctgatcagcctcgactgtgccttctagttg
Humanized C2 gBLOCK#8 sequence (lambda light chain in pCDNA3.1 V5):
(DNA)
(SEQ ID NO: 216)
actcactatagggagacccaagctggctagttaagcttgggccaccatggagacagacacactcctgctatgggtactgct
gctctgggttccaggttccactggtgacgacattgtgctgacccagtctccagcctccttggccgtgtctccaggacagag
ggccaccatcacctgcagagccagtaagagtgtcagtaccagcggatactcctacatgcactggtatcagcagaaaccagg
acaacctcctaaactcctgatttacctggcatccaatctggagagcggggtcccagccaggttcagcggcagtgggtctgg
gaccgatttcaccctcacaattaatcctgtggaagctaatgatactgcaaattattactgtcagcacagtagggagctgcc
tttcacattcggcggagggaccaaggtggagatcaaacgaactggtcagcccaaggctgccccctcggtcactctgttccc
gccctcctctgaggagcttcaagccaacaaggccacactggtgtgtctcataagtgacttctacccgggagccgtgacagt
ggcctggaaggcagatagcagccccgtcaaggcgggagtggagaccaccacaccctccaaacaaagcaacaacaagtacgc
ggccagcagctatctgagcctgacgcctgagcagtggaagtcccacagaagctacagctgccaggtcacgcatgaagggag
caccgtggagaagacagtggcccctacagaatgttcatagtaagtttaaacccgctgatcagcctcgactgtgccttctag
ttg
pCDNA3.1 V5 5′ overlapping sequence:
(DNA)
(SEQ ID NO: 217)
actcactatagggagacccaagctggctagtt
pCDNA3.1 V5 3′ overlapping sequence:
(DNA)
(SEQ ID NO: 218)
ccgctgatcagcctcgactgtgccttctagttg
Humanized C2 gBLOCK#9 sequence (lambda light chain in pSEC Tag2):
(DNA)
(SEQ ID NO: 219)
tgctctgggttccaggttccactggtgacgcggcccagccggccgacattgtgctgacccagtctccagcctccttggccg
tgtctccaggacagagggccaccatcacctgcagagccagtaagagtgtcagtaccagcggatactcctacatgcactggt
atcagcagaaaccaggacaacctcctaaactcctgatttacctggcatccaatctggagagcggggtcccagccaggttca
gcggcagtgggtctgggaccgatttcaccctcacaattaatcctgtggaagctaatgatactgcaaattattactgtcagc
acagtagggagctgcctttcacattcggcggagggaccaaggtggagatcaaacgaactggtcagcccaaggctgccccct
cggtcactctgttcccgccctcctctgaggagcttcaagccaacaaggccacactggtgtgtctcataagtgacttctacc
cgggagccgtgacagtggcctggaaggcagatagcagccccgtcaaggcgggagtggagaccaccacaccctccaaacaaa
gcaacaacaagtacgcggccagcagctatctgagcctgacgcctgagcagtggaagtcccacagaagctacagctgccagg
tcacgcatgaagggagcaccgtggagaagacagtggcccctacagaatgttcatagtaagtttaaacccgctgatcagcct
cgactgtgccttctagttg
pSEC Tag2 5′ overlapping sequence:
(DNA)
(SEQ ID NO: 220)
tgctctgggttccaggttccactggtgacgc
pSEC Tag2 3′ overlapping sequence:
(DNA)
(SEQ ID NO: 221)
ccgctgatcagcctcgactgtgccttctagttg
Murine Ig kappa chain leader sequence
(DNA)
(SEQ ID NO: 222)
atggagacagacacactcctgctatgggtactgctgctctgggttccaggttccactggtgac
(amino acids)
(SEQ ID NO: 223)
METDTLLLWVLLLWVPGSTGD
Interleukin-2 (IL-2) leader sequence
(DNA)
(SEQ ID NO: 224)
atgtacaggatgcaactcctgtcttgcattgcactaagtcttgcacttgtcacaaacagt
(amino acids)
(SEQ ID NO: 225)
MYRMQLLSCIALSLALVTNS
CD33 leader sequence
(DNA)
(SEQ ID NO: 226)
atgcctcttctgcttctgcttcctctgctttgggctggagctcttgct
(amino acids)
(SEQ ID NO: 227)
MPLLLLLPLLWAGALA
IGHV3-21*03 leader sequence
(DNA)
(SEQ ID NO: 228)
atggaactggggctccgctgggttttccttgttgctattttagaaggtgtccagtgt
(amino acids)
(SEQ ID NO: 229)
MELGLRWVFLVAILEGVQC
IGHV3-11*02 leader sequence
(DNA)
(SEQ ID NO: 230)
atggaagccccagcgcagcttctcttcctcctgctactctggctcccagataccactgga
(amino acids)
(SEQ ID NO: 231)
MEAPAQLLFLLLLWLPDTTG
Humanized E6 single chain GS3
(DNA)
(SEQ ID NO: 232)
gaggtgcagctggtggagtctgggggaggcctggtcaagcctggggggtccctgagactctcctgtgcagcctctggattc
accttcagtaggtatggcatgagctgggtccgccaggctccagggaagaggctggagtgggtctcaaccattagtggcgga
ggcacctacatatactacccagactcagtgaagggccgattcaccatctccagagacaacgccaagaacaccctgtatctg
caaatgaacagcctgagagccgaggacacggctgtgtattactgtaccagagataactatggccgcaactatgattatggc
atggattattggggccagggcaccctggtgaccgtgagcagcggcggtggcggatccggcggtggcggatccggcggtggc
ggatccgaaattgtgttgacacagtctccagccaccctgtctttgtctccaggggaaagagccaccctcacctgcagcgcc
accagcagtgttagctacatccactggtaccaacagaggcctggccagagccccaggctcctcatctatagcacctccaac
ctggccagcggcatcccagccaggttcagtggcagtgggtctgggagcgactacactctcaccatcagcagcctagagcct
gaagattttgcagtttattactgtcagcagcgtagcagctcccctttcacctttggcagcggcaccaaagtggaaattaaa
(amino acids)
(SEQ ID NO: 233)
EVQLVESGGGLVKPGGSLRLSCAASGFTFSRYGMSWVRQAPGKRLEWVSTISGGGTYIYYPDSVKGRFTISRDNAKNTLY
LQMNSLRAEDTAVYYCTRDNYGRNYDYGMDYWGQGTLVTVSSGGGGSGGGGSGGGGSEIVLTQSPATLSLSPGERATLTC
SATSSVSYIHWYQQRPGQSPRLLIYSTSNLASGIPARFSGSGSGSDYTLTISSLEPEDFAVYYCQQRSSSPFTFGSGTKV
EIK
Humanized E6 single chain IgGlnoC
(DNA)
(SEQ ID NO: 234)
gaggtgcagctggtggagtctgggggaggcctggtcaagcctggggggtccctgagactctcctgtgcagcctctggattc
accttcagtaggtatggcatgagctgggtccgccaggctccagggaagaggctggagtgggtctcaaccattagtggcgga
ggcacctacatatactacccagactcagtgaagggccgattcaccatctccagagacaacgccaagaacaccctgtatctg
caaatgaacagcctgagagccgaggacacggctgtgtattactgtaccagagataactatggccgcaactatgattatggc
atggattattggggccagggcaccctggtgaccgtgagcagcgataaaacccatactaaaccgccaaaaccggcgccggaa
ctgctgggtggtcctggtaccggtgaaattgtgttgacacagtctccagccaccctgtctttgtctccaggggaaagagcc
accctcacctgcagcgccaccagcagtgttagctacatccactggtaccaacagaggcctggccagagccccaggctcctc
atctatagcacctccaacctggccagcggcatcccagccaggttcagtggcagtgggtctgggagcgactacactctcacc
atcagcagcctagagcctgaagattttgcagtttattactgtcagcagcgtagcagctcccctttcacctttggcagcggc
accaaagtggaaattaaa
(amino acids)
(SEQ ID NO: 235)
EVQLVESGGGLVKPGGSLRLSCAASGFTFSRYGMSWVRQAPGKRLEWVSTISGGGTYIYYPDSVKGRFTISRDNAKNTLY
LQMNSLRAEDTAVYYCTRDNYGRNYDYGMDYWGQGTLVTVSSDKTHTKPPKPAPELLGGPGTGEIVLTQSPATLSLSPGE
RATLTCSATSSVSYIHWYQQRPGQSPRLLIYSTSNLASGIPARFSGSGSGSDYTLTISSLEPEDFAVYYCQQRSSSPFTF
GSGTKVEIK
Humanized E6 single chain X4 (linker is IgG1 and IgG2 modified hinge region)
(DNA)
(SEQ ID NO: 236)
gaggtgcagctggtggagtctgggggaggcctggtcaagcctggggggtccctgagactctcctgtgcagcctctggattc
accttcagtaggtatggcatgagctgggtccgccaggctccagggaagaggctggagtgggtctcaaccattagtggcgga
ggcacctacatatactacccagactcagtgaagggccgattcaccatctccagagacaacgccaagaacaccctgtatctg
caaatgaacagcctgagagccgaggacacggctgtgtattactgtaccagagataactatggccgcaactatgattatggc
atggattattggggccagggcaccctggtgaccgtgagcagcgataaaacccatactaaaccgccaaaaccggcgccggaa
ctgctgggtggtcctggtaccggtactggtggtccgactattaaacctccgaaacctccgaaacctgctccgaacctgctg
ggtggtccggaaattgtgttgacacagtctccagccaccctgtctttgtctccaggggaaagagccaccctcacctgcagc
gccaccagcagtgttagctacatccactggtaccaacagaggcctggccagagccccaggctcctcatctatagcacctcc
aacctggccagcggcatcccagccaggttcagtggcagtgggtctgggagcgactacactctcaccatcagcagcctagag
cctgaagattttgcagtttattactgtcagcagcgtagcagctcccctttcacctttggcagcggcaccaaagtggaaatt
aaa
(amino acids)
(SEQ ID NO: 237)
EVQLVESGGGLVKPGGSLRLSCAASGFTFSRYGMSWVRQAPGKRLEWVSTISGGGTYIYYPDSVKGRFTISRDNAKNTLY
LQMNSLRAEDTAVYYCTRDNYGRNYDYGMDYWGQGTLVTVSSDKTHTKPPKPAPELLGGPGTGTGGPTIKPPKPPKPAPN
LLGGPEIVLTQSPATLSLSPGERATLTCSATSSVSYIHWYQQRPGQSPRLLIYSTSNLASGIPARFSGSGSGSDYTLTIS
SLEPEDFAVYYCQQRSSSPFTFGSGTKVEIK
Humanized C2 single chain GS3
(DNA)
(SEQ ID NO: 238)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaact
(amino acids)
(SEQ ID NO: 239)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRT
Humanized C2 single chain IgG (no Cysteine)
(DNA)
(SEQ ID NO: 240)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccgataaaacccatactaaaccgccaaaaccggcgccggaactg
ctgggtggtcctggtaccggtgacattgtgctgacccagtctccagcctccttggccgtgtctccaggacagagggccacc
atcacctgcagagccagtaagagtgtcagtaccagcggatactcctacatgcactggtatcagcagaaaccaggacaacct
cctaaactcctgatttacctggcatccaatctggagagcggggtcccagccaggttcagcggcagtgggtctgggaccgat
ttcaccctcacaattaatcctgtggaagctaatgatactgcaaattattactgtcagcacagtagggagctgcctttcaca
ttcggcggagggaccaaggtggagatcaaacgaact
(amino acids)
(SEQ ID NO: 241)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSDKTHTKPPKPAPELLGGPGTGDIVLTQSPASLAVSPGQR
ATITCRASKSVSTSGYSYMHWYQQKPGQPPKLLIYLASNLESGVPARFSGSGSGTDFTLTINPVEANDTANYYCQHSREL
PFTFGGGTKVEIKRT
Humanized C2 single chain X4 (linker is IgG1 and IgG2 modified hinge region)
(DNA)
(SEQ ID NO: 242)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccgataaaacccatactaaaccgccaaaaccggcgccggaactg
ctgggtggtcctggtaccggtactggtggtccgactattaaacctccgaaacctccgaaacctgctccgaacctgctgggt
ggtccggacattgtgctgacccagtctccagcctccttggccgtgtctccaggacagagggccaccatcacctgcagagcc
agtaagagtgtcagtaccagcggatactcctacatgcactggtatcagcagaaaccaggacaacctcctaaactcctgatt
tacctggcatccaatctggagagcggggtcccagccaggttcagcggcagtgggtctgggaccgatttcaccctcacaatt
aatcctgtggaagctaatgatactgcaaattattactgtcagcacagtagggagctgcctttcacattcggcggagggacc
aaggtggagatcaaacgaact
(amino acids)
(SEQ ID NO: 243)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSDKTHTKPPKPAPELLGGPGTGTGGPTIKPPKPPKPAPNL
LGGPDIVLTQSPASLAVSPGQRATITCRASKSVSTSGYSYMHWYQQKPGQPPKLLIYLASNLESGVPARFSGSGSGTDFT
LTINPVEANDTANYYCQHSRELPFTFGGGTKVEIKRT
Humanized C3 single chain GS3
(DNA)
(SEQ ID NO: 244)
caggttcagctggtgcagtctggagctgaggtgaagaagcctggggcctcagtgaaggtctcctgcaaggcttctggttac
acctttaccgactacgccatgaactgggtgcgacaggcccctggacaagggcttgagtggatgggagtgatcagcaccttc
agcggtaacacaaacttcaaccagaagttcaagggcagagtcaccatgaccacagacacatccacgagcacagcctacatg
gagctgaggagcctgagatctgacgacacggccgtgtattactgtgcgagaagcgactactacggcccatacttcgactac
tggggccagggcaccaccctgaccgtgtccagcggcggtggcggatccggcggtggcggatccggcggtggcggatccgat
attgtgatgacccagactccactctctctgtccgtcacccctggacagccggcctccatctcctgcaggtctagtcagacc
attgtccatagtaatggaaacacctatttggagtggtacctgcagaagccaggccagtctccacagctcctgatctataag
gtttccaaccggttctctggagtgccagataggttcagtggcagcgggtcagggacagatttcacactgaaaatcagccgg
gtggaggctgaggatgttggggtttattactgcttccaaggtagccacgtgcctttcaccttcggcggagggaccaaggtg
gagatcaaacgaact
(amino acids)
(SEQ ID NO: 245)
QVQLVQSGAEVKKPGASVKVSCKASGYTFTDYAMNWVRQAPGQGLEWMGVISTFSGNTNFNQKFKGRVTMTTDTSTSTAYM
ELRSLRSDDTAVYYCARSDYYGPYFDYWGQGTTLTVSSGGGGSGGGGSGGGGSDIVMTQTPLSLSVTPGQPASISCRSSQ
TIVHSNGNTYLEWYLQKPGQSPQLLIYKVSNRFSGVPDRFSGSGSGTDFTLKISRVEAEDVGVYYCFQGSHVPFTFGGGT
KVEIKRT
Humanized C3 single chain IgG1 (no Cysteine)
(DNA)
(SEQ ID NO: 246)
caggttcagctggtgcagtctggagctgaggtgaagaagcctggggcctcagtgaaggtctcctgcaaggcttctggttac
acctttaccgactacgccatgaactgggtgcgacaggcccctggacaagggcttgagtggatgggagtgatcagcaccttc
agcggtaacacaaacttcaaccagaagttcaagggcagagtcaccatgaccacagacacatccacgagcacagcctacatg
gagctgaggagcctgagatctgacgacacggccgtgtattactgtgcgagaagcgactactacggcccatacttcgactac
tggggccagggcaccaccctgaccgtgtccagcgataaaacccatactaaaccgccaaaaccggcgccggaactgctgggt
ggtcctggtaccggtgatattgtgatgacccagactccactctctctgtccgtcacccctggacagccggcctccatctcc
tgcaggtctagtcagaccattgtccatagtaatggaaacacctatttggagtggtacctgcagaagccaggccagtctcca
cagctcctgatctataaggtttccaaccggttctctggagtgccagataggttcagtggcagcgggtcagggacagatttc
acactgaaaatcagccgggtggaggctgaggatgttggggtttattactgcttccaaggtagccacgtgcctttcaccttc
ggcggagggaccaaggtggagatcaaacgaact
(amino acids)
(SEQ ID NO: 247)
QVQLVQSGAEVKKPGASVKVSCKASGYTFTDYAMNWVRQAPGQGLEWMGVISTFSGNTNFNQKFKGRVTMTTDTSTSTAYM
ELRSLRSDDTAVYYCARSDYYGPYFDYWGQGTTLTVSSDKTHTKPPKPAPELLGGPGTGDIVMTQTPLSLSVTPGQPASI
SCRSSQTIVHSNGNTYLEWYLQKPGQSPQLLIYKVSNRFSGVPDRFSGSGSGTDFTLKISRVEAEDVGVYYCFQGSHVPF
TFGGGTKVEIKRT
Humanized C3 single chain X4 (linker is IgG1 and IgG2 modified hinge region)
(DNA)
(SEQ ID NO: 248)
caggttcagctggtgcagtctggagctgaggtgaagaagcctggggcctcagtgaaggtctcctgcaaggcttctggttac
acctttaccgactacgccatgaactgggtgcgacaggcccctggacaagggcttgagtggatgggagtgatcagcaccttc
agcggtaacacaaacttcaaccagaagttcaagggcagagtcaccatgaccacagacacatccacgagcacagcctacatg
gagctgaggagcctgagatctgacgacacggccgtgtattactgtgcgagaagcgactactacggcccatacttcgactac
tggggccagggcaccaccctgaccgtgtccagcgataaaacccatactaaaccgccaaaaccggcgccggaactgctgggt
ggtcctggtaccggtactggtggtccgactattaaacctccgaaacctccgaaacctgctccgaacctgctgggtggtccg
gatattgtgatgacccagactccactctctctgtccgtcacccctggacagccggcctccatctcctgcaggtctagtcag
accattgtccatagtaatggaaacacctatttggagtggtacctgcagaagccaggccagtctccacagctcctgatctat
aaggtttccaaccggttctctggagtgccagataggttcagtggcagcgggtcagggacagatttcacactgaaaatcagc
cgggtggaggctgaggatgttggggtttattactgcttccaaggtagccacgtgcctttcaccttcggcggagggaccaag
gtggagatcaaacgaact
(amino acids)
(SEQ ID NO: 249)
QVQLVQSGAEVKKPGASVKVSCKASGYTFTDYAMNWVRQAPGQGLEWMGVISTFSGNTNFNQKFKGRVTMTTDTSTSTAYM
ELRSLRSDDTAVYYCARSDYYGPYFDYWGQGTTLTVSSDKTHTKPPKPAPELLGGPGTGTGGPTIKPPKPPKPAPNLLGG
PDIVMTQTPLSLSVTPGQPASISCRSSQTIVHSNGNTYLEWYLQKPGQSPQLLIYKVSNRFSGVPDRFSGSGSGTDFTLK
ISRVEAEDVGVYYCFQGSHVPFTFGGGTKVEIKRT
Humanized C8 single chain GS3 (linker is [Gly₄Ser₁]₃)
(DNA)
(SEQ ID NO: 250)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccctgactcagtgaagggccgattcaccatctccagagacaacgccaagaactcactgtatctg
caaatgaacagcctgagagccgaggacacggccgtgtattactgtgcgagactgggcggcgataactattatgaatattgg
ggcaaagggaccacggtcaccgtctcctccggcggtggcggatccggcggtggcggatccggcggtggcggatccgacatc
gtgatgacccagtctccagactccctggctgtgtctctgggcgagagggccaccatcaactgcagggccagcaagagtgtt
agcaccagcggctacagctacatgcactggtaccagcagaaaccaggacagcctcctaagctgctcatttacctggtgtct
aacctggaatccggggtccctgaccgattcagtggcagcgggtctgggacagatttcactctcaccatcagcagcctgcag
gctgaagatgtggcagtttattactgtcaacacattcgggaactgaccaggagtgaattcggcggagggaccaaggtggag
atcaaacgaact
(amino acids)
(SEQ ID NO: 251)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYWGKGTTVTVSSGGGGSGGGGSGGGGSDIVMTQSPDSLAVSLGERATINCRASK
SVSTSGYSYMHWYQQKPGQPPKLLIYLVSNLESGVPDRFSGSGSGTDFTLTISSLQAEDVAVYYCQHIRELTRSEFGGGT
KVEIKRT
Humanized C8 single chain IgG1 (no Cysteine)
(DNA)
(SEQ ID NO: 252)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccctgactcagtgaagggccgattcaccatctccagagacaacgccaagaactcactgtatctg
caaatgaacagcctgagagccgaggacacggccgtgtattactgtgcgagactgggcggcgataactattatgaatattgg
ggcaaagggaccacggtcaccgtctcctccgataaaacccatactaaaccgccaaaaccggcgccggaactgctgggtggt
cctggtaccggtgacatcgtgatgacccagtctccagactccctggctgtgtctctgggcgagagggccaccatcaactgc
agggccagcaagagtgttagcaccagcggctacagctacatgcactggtaccagcagaaaccaggacagcctcctaagctg
ctcatttacctggtgtctaacctggaatccggggtccctgaccgattcagtggcagcgggtctgggacagatttcactctc
accatcagcagcctgcaggctgaagatgtggcagtttattactgtcaacacattcgggaactgaccaggagtgaattcggc
ggagggaccaaggtggagatcaaacgaact
(amino acids)
(SEQ ID NO: 253)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYWGKGTTVTVSSDKTHTKPPKPAPELLGGPGTGDIVMTQSPDSLAVSLGERATI
NCRASKSVSTSGYSYMHWYQQKPGQPPKLLIYLVSNLESGVPDRFSGSGSGTDFTLTISSLQAEDVAVYYCQHIRELTRS
EFGGGTKVEIKRT
Humanized C8 single chain X4 (linker is IgG1 and IgG2 modified hinge region)
(DNA)
(SEQ ID NO: 254)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactacccagactcagtgaagggccgattcaccatctccagagacaacgccaagaactcactgtatctg
caaatgaacagcctgagagccgaggacacggccgtgtattactgtgcgagactgggcggcgacaattactatgagtattgg
ggcaaagggaccacggtcaccgtctcctccgataaaacccatactaaaccgccaaaaccggcgccggaactgctgggtggt
cctggtaccggtactggtggtccgactattaaacctccgaaacctccgaaacctgctccgaacctgctgggtggtccggac
atcgtgatgacccagtctccagactccctggctgtgtctctgggcgagagggccaccatcaactgcagggccagcaagagt
gttagcaccagcggctacagctacatgcactggtaccagcagaaaccaggacagcctcctaagctgctcatttacctggtg
tctaacctggaatccggggtccctgaccgattcagtggcagcgggtctgggacagatttcactctcaccatcagcagcctg
caggctgaagatgtggcagtttattactgtcaacacattcgggaactgaccaggagtgaattcggcggagggaccaaggtg
gagatcaaacgaact
(amino acids)
(SEQ ID NO: 255)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYWGKGTTVTVSSDKTHTKPPKPAPELLGGPGTGTGGPTIKPPKPPKPAPNLLGG
PDIVMTQSPDSLAVSLGERATINCRASKSVSTSGYSYMHWYQQKPGQPPKLLIYLVSNLESGVPDRFSGSGSGTDFTLTI
SSLQAEDVAVYYCQHIRELTRSEFGGGTKVEIKRT
pSECTag2 E6 scFV-FC
(DNA)
(SEQ ID NO: 256)
atggagacagacacactcctgctatgggtactgctgctctgggttccaggttccactggtgacgcggcccagccggccgag
gtgcagctggtggagtctgggggaggcctggtcaagcctggggggtccctgagactctcctgtgcagcctctggattcacc
ttcagtaggtatggcatgagctgggtccgccaggctccagggaagaggctggagtgggtctcaaccattagtggcggaggc
acctacatatactacccagactcagtgaagggccgattcaccatctccagagacaacgccaagaacaccctgtatctgcaa
atgaacagcctgagagccgaggacacggctgtgtattactgtaccagagataactatggccgcaactatgattatggcatg
gattattggggccagggcaccctggtgaccgtgagcagcggcggtggcggatccggcggtggcggatccggcggtggcgga
tccgaaattgtgttgacacagtctccagccaccctgtctttgtctccaggggaaagagccaccctcacctgcagcgccacc
agcagtgttagctacatccactggtaccaacagaggcctggccagagccccaggctcctcatctatagcacctccaacctg
gccagcggcatcccagccaggttcagtggcagtgggtctgggagcgactacactctcaccatcagcagcctagagcctgaa
gattttgcagtttattactgtcagcagcgtagcagctcccctttcacctttggcagcggcaccaaagtggaaattaaagag
cccaaatcttgtgacaaaactcacacatgcccaccgtgcccagcacctgaactcctggggggaccgtcagtcttcctcttc
cccccaaaacccaaggacaccctcatgatctcccggacccctgaggtcacatgcgtggtggtggacgtgagccacgaagac
cctgaggtcaagttcaactggtacgtggacggcgtggaggtgcataatgccaagacaaagccgcgggaggagcagtacaac
agcacgtaccgtgtggtcagcgtcctcaccgtcctgcaccaggactggctgaatggcaaggagtacaagtgcaaggtctcc
aacaaagccctcccagcccccatcgagaaaaccatctccaaagccaaagggcagccccgagaaccacaggtgtacaccctg
cccccatcccgggaggagatgaccaagaaccaggtcagcctgacctgcctggtcaaaggcttctatcccagcgacatcgcc
gtggagtgggagagcaatgggcagccggagaacaactacaagaccacgcctcccgtgctggactccgacggctccttcttc
ctctacagcaagctcaccgtggacaagagcaggtggcagcaggggaacgtcttctcatgctccgtgatgcatgaggctctg
cacaaccactacacgcagaagagcctctccctgtctccgggtaaatgataa
(amino acids)
(SEQ ID NO: 257)
METDTLLLWVLLLWVPGSTGDAAQPAEVQLVESGGGLVKPGGSLRLSCAASGFTFSRYGMSWVRQAPGKRLEWVSTISGG
GTYIYYPDSVKGRFTISRDNAKNTLYLQMNSLRAEDTAVYYCTRDNYGRNYDYGMDYWGQGTLVTVSSGGGGSGGGGSGG
GGSEIVLTQSPATLSLSPGERATLTCSATSSVSYIHWYQQRPGQSPRLLIYSTSNLASGIPARFSGSGSGSDYTLTISSL
EPEDFAVYYCQQRSSSPFTFGSGTKVEIKEPKSCDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSRWQQGNVFSCS
VMHEALHNHYTQKSLSLSPGK**
E6 scFC-FC 1 gBLOCk sequence:
(SEQ ID NO: 258)
tgctctgggttccaggttccactggtgacgcggcccagccggccgaggtgcagctggtggagtctgggggaggcctggtca
agcctggggggtccctgagactctcctgtgcagcctctggattcaccttcagtaggtatggcatgagctgggtccgccagg
ctccagggaagaggctggagtgggtctcaaccattagtggcggaggcacctacatatactacccagactcagtgaagggcc
gattcaccatctccagagacaacgccaagaacaccctgtatctgcaaatgaacagcctgagagccgaggacacggctgtgt
attactgtaccagagataactatggccgcaactatgattatggcatggattattggggccagggcaccctggtgaccgtga
gcagcggcggtggcggatccggcggtggcggatccggcggtggcggatccgaaattgtgttgacacagtctccagccaccc
tgtctttgtc
E6 scFC-FC 2 gBLOCk sequence:
(SEQ ID NO: 259)
aattgtgttgacacagtctccagccaccctgtctttgtctccaggggaaagagccaccctcacctgcagcgccaccagcag
tgttagctacatccactggtaccaacagaggcctggccagagccccaggctcctcatctatagcacctccaacctggccag
cggcatcccagccaggttcagtggcagtgggtctgggagcgactacactctcaccatcagcagcctagagcctgaagattt
tgcagtttattactgtcagcagcgtagcagctcccctttcacctttggcagcggcaccaaagtggaaattaaagagcccaa
atcttgtgacaaaactcacacatgcccaccgtgcccagcacctgaactcctggggggaccgtcagtcttcctcttcccccc
aaaacccaaggacaccctcatgatctcccggacccctgaggtcacatgcgtggtggtggacgtgagccacgaagaccctga
ggtcaagttcaactggtacgtggacggcgtggaggtgcataatgccaagacaaagccgcgggaggagcagtacaacagcac
gtaccgtgtggtcagc
pSECTag2 C2 scFV-FC
(DNA)
(SEQ ID NO: 260)
atggagacagacacactcctgctatgggtactgctgctctgggttccaggttccactggtgacgcggcccagccggccgag
gtgcagctggtggagtctgggggaggcctggtcaagcctggggggtccctgagactctcctgtgcagcctctggattcacc
ttcagtggctatgccatgagctgggtccgccaggctccagggaaggggctggagtgggtctcaaccattagtagtggcgga
acctacatatactaccccgactcagtgaagggccgattcaccatctccagagacaacgccaagaactcactgtatctgcaa
atgaacagcctgagagccgaggacacggccgtgtattactgtgcgagacttgggggggataattactacgaatacttcgat
gtctggggcaaagggaccacggtcaccgtctcctccggcggtggcggatccggcggtggcggatccggcggtggcggatcc
gacattgtgctgacccagtctccagcctccttggccgtgtctccaggacagagggccaccatcacctgcagagccagtaag
agtgtcagtaccagcggatactcctacatgcactggtatcagcagaaaccaggacaacctcctaaactcctgatttacctg
gcatccaatctggagagcggggtcccagccaggttcagcggcagtgggtctgggaccgatttcaccctcacaattaatcct
gtggaagctaatgatactgcaaattattactgtcagcacagtagggagctgcctttcacattcggcggagggaccaaggtg
gagatcaaacgaactgagcccaaatcttgtgacaaaactcacacatgcccaccgtgcccagcacctgaactcctgggggga
ccgtcagtcttcctcttccccccaaaacccaaggacaccctcatgatctcccggacccctgaggtcacatgcgtggtggtg
gacgtgagccacgaagaccctgaggtcaagttcaactggtacgtggacggcgtggaggtgcataatgccaagacaaagccg
cgggaggagcagtacaacagcacgtaccgtgtggtcagcgtcctcaccgtcctgcaccaggactggctgaatggcaaggag
tacaagtgcaaggtctccaacaaagccctcccagcccccatcgagaaaaccatctccaaagccaaagggcagccccgagaa
ccacaggtgtacaccctgcccccatcccgggaggagatgaccaagaaccaggtcagcctgacctgcctggtcaaaggcttc
tatcccagcgacatcgccgtggagtgggagagcaatgggcagccggagaacaactacaagaccacgcctcccgtgctggac
tccgacggctccttcttcctctacagcaagctcaccgtggacaagagcaggtggcagcaggggaacgtcttctcatgctcc
gtgatgcatgaggctctgcacaaccactacacgcagaagagcctctccctgtctccgggtaaatgataa
(amino acids)
(SEQ ID NO: 261)
METDTLLLWVLLLWVPGSTGDAAQPAEVQLVESGGGLVKPGGSLRLSCAASGFTFSGYAMSWVRQAPGKGLEWVSTISSG
GTYIYYPDSVKGRFTISRDNAKNSLYLQMNSLRAEDTAVYYCARLGGDNYYEYFDVWGKGTTVTVSSGGGGSGGGGSGGG
GSDIVLTQSPASLAVSPGQRATITCRASKSVSTSGYSYMHWYQQKPGQPPKLLIYLASNLESGVPARFSGSGSGTDFTLT
INPVEANDTANYYCQHSRELPFTFGGGTKVEIKRTEPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLDSDGSFFLYSKLTVDKSRWQQG
NVFSCSVMHEALHNHYTQKSLSLSPGK**
C2 scFV-FC 1 gBLOCk sequence:
(DNA)
(SEQ ID NO: 262)
tgctctgggttccaggttccactggtgacgcggcccagccggccgaggtgcagctggtggagtctgggggaggcctggtca
agcctggggggtccctgagactctcctgtgcagcctctggattcaccttcagtggctatgccatgagctgggtccgccagg
ctccagggaaggggctggagtgggtctcaaccattagtagtggcggaacctacatatactaccccgactcagtgaagggcc
gattcaccatctccagagacaacgccaagaactcactgtatctgcaaatgaacagcctgagagccgaggacacggccgtgt
attactgtgcgagacttgggggggataattactacgaatacttcgatgtctggggcaaagggaccacggtcaccgtctcct
ccggcggtggcggatccggcggtggcggatccggcggtggcggatccgacattgtgctgacccagtctccagcctccttgg
c
C2 scFV-FC 2 gBLOCk sequence:
(DNA)
(SEQ ID NO: 263)
cattgtgctgacccagtctccagcctccttggccgtgtctccaggacagagggccaccatcacctgcagagccagtaagag
tgtcagtaccagcggatactcctacatgcactggtatcagcagaaaccaggacaacctcctaaactcctgatttacctggc
atccaatctggagagcggggtcccagccaggttcagcggcagtgggtctgggaccgatttcaccctcacaattaatcctgt
ggaagctaatgatactgcaaattattactgtcagcacagtagggagctgcctttcacattcggcggagggaccaaggtgga
gatcaaacgaactgagcccaaatcttgtgacaaaactcacacatgcccaccgtgcccagcacctgaactcctggggggacc
gtcagtcttcctcttccccccaaaacccaaggacaccctcatgatctcccggacccctgaggtcacatgcgtggtggtgga
cgtgagccacgaagaccctgaggtcaagttcaactggtacgtggacggcgtggaggtgcataatgccaagacaaagccgcg
ggaggagcagtacaacagcacgtaccgtgtggtcagc
pSECTag2 C3 scFV-FC
(DNA)
(SEQ ID NO: 264)
atggagacagacacactcctgctatgggtactgctgctctgggttccaggttccactggtgacgcggcccagccggcccag
gttcagctggtgcagtctggagctgaggtgaagaagcctggggcctcagtgaaggtctcctgcaaggcttctggttacacc
tttaccgactacgccatgaactgggtgcgacaggcccctggacaagggcttgagtggatgggagtgatcagcaccttcagc
ggtaacacaaacttcaaccagaagttcaagggcagagtcaccatgaccacagacacatccacgagcacagcctacatggag
ctgaggagcctgagatctgacgacacggccgtgtattactgtgcgagaagcgactactacggcccatacttcgactactgg
ggccagggcaccaccctgaccgtgtccagcggcggtggcggatccggcggtggcggatccggcggtggcggatccgatatt
gtgatgacccagactccactctctctgtccgtcacccctggacagccggcctccatctcctgcaggtctagtcagaccatt
gtccatagtaatggaaacacctatttggagtggtacctgcagaagccaggccagtctccacagctcctgatctataaggtt
tccaaccggttctctggagtgccagataggttcagtggcagcgggtcagggacagatttcacactgaaaatcagccgggtg
gaggctgaggatgttggggtttattactgcttccaaggtagccacgtgcctttcaccttcggcggagggaccaaggtggag
atcaaacgaactgagcccaaatcttgtgacaaaactcacacatgcccaccgtgcccagcacctgaactcctggggggaccg
tcagtcttcctcttccccccaaaacccaaggacaccctcatgatctcccggacccctgaggtcacatgcgtggtggtggac
gtgagccacgaagaccctgaggtcaagttcaactggtacgtggacggcgtggaggtgcataatgccaagacaaagccgcgg
gaggagcagtacaacagcacgtaccgtgtggtcagcgtcctcaccgtcctgcaccaggactggctgaatggcaaggagtac
aagtgcaaggtctccaacaaagccctcccagcccccatcgagaaaaccatctccaaagccaaagggcagccccgagaacca
caggtgtacaccctgcccccatcccgggaggagatgaccaagaaccaggtcagcctgacctgcctggtcaaaggcttctat
cccagcgacatcgccgtggagtgggagagcaatgggcagccggagaacaactacaagaccacgcctcccgtgctggactcc
gacggctccttcttcctctacagcaagctcaccgtggacaagagcaggtggcagcaggggaacgtcttctcatgctccgtg
atgcatgaggctctgcacaaccactacacgcagaagagcctctccctgtctccgggtaaatgataa
(amino acids)
(SEQ ID NO: 265)
METDTLLLWVLLLWVPGSTGDAAQPAQVQLVQSGAEVKKPGASVKVSCKASGYTFTDYAMNWVRQAPGQGLEWMGVISTF
SGNTNFNQKFKGRVTMTTDTSTSTAYMELRSLRSDDTAVYYCARSDYYGPYFDYWGQGTTLTVSSGGGGSGGGGSGGGGS
DIVMTQTPLSLSVTPGQPASISCRSSQTIVHSNGNTYLEWYLQKPGQSPQLLIYKVSNRFSGVPDRFSGSGSGTDFTLKI
SRVEAEDVGVYYCFQGSHVPFTFGGGTKVEIKRTEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENNYKTTPPVLDSDGSFFLYSKLTVDKSRWQQGN
VFSCSVMHEALHNHYTQKSLSLSPGK**
C3 GS scFV FC 1 gBLOCk sequence:
(DNA)
(SEQ ID NO: 266)
tgctctgggttccaggttccactggtgacgcggcccagccggcccaggttcagctggtgcagtctggagctgaggtgaaga
agcctggggcctcagtgaaggtctcctgcaaggcttctggttacacctttaccgactacgccatgaactgggtgcgacagg
cccctggacaagggcttgagtggatgggagtgatcagcaccttcagcggtaacacaaacttcaaccagaagttcaagggca
gagtcaccatgaccacagacacatccacgagcacagcctacatggagctgaggagcctgagatctgacgacacggccgtgt
attactgtgcgagaagcgactactacggcccatacttcgactactggggccagggcaccaccctgaccgtgtccagcggcg
gtggcggatccggcggtggcggatccggcggtggcggatccgatattgtgatgacccagactccactctctctgt
C3 scFV FC2 gBLOCk sequence:
(DNA)
(SEQ ID NO: 267)
tattgtgatgacccagactccactctctctgtccgtcacccctggacagccggcctccatctcctgcaggtctagtcagac
cattgtccatagtaatggaaacacctatttggagtggtacctgcagaagccaggccagtctccacagctcctgatctataa
ggtttccaaccggttctctggagtgccagataggttcagtggcagcgggtcagggacagatttcacactgaaaatcagccg
ggtggaggctgaggatgttggggtttattactgcttccaaggtagccacgtgcctttcaccttcggcggagggaccaaggt
ggagatcaaacgaactgagcccaaatcttgtgacaaaactcacacatgcccaccgtgcccagcacctgaactcctgggggg
accgtcagtcttcctcttccccccaaaacccaaggacaccctcatgatctcccggacccctgaggtcacatgcgtggtggt
ggacgtgagccacgaagaccctgaggtcaagttcaactggtacgtggacggcgtggaggtgcataatgccaagacaaagcc
gcgggaggagcagtacaacagcacgtaccgtgtggtcagc
pSECTag2 C8 scFV-FC
(DNA)
(SEQ ID NO: 268)
atggagacagacacactcctgctatgggtactgctgctctgggttccaggttccactggtgacgcggcccagccggccgag
gtgcagctggtggagtctgggggaggcctggtcaagcctggggggtccctgagactctcctgtgcagcctctggattcacc
ttcagtggctatgccatgagctgggtccgccaggctccagggaaggggctggagtgggtctcaaccattagtagtggcgga
acctacatatactaccctgactcagtgaagggccgattcaccatctccagagacaacgccaagaactcactgtatctgcaa
atgaacagcctgagagccgaggacacggccgtgtattactgtgcgagactgggcggcgataactattatgaatattggggc
aaagggaccacggtcaccgtctcctccggcggtggcggatccggcggtggcggatccggcggtggcggatccgacatcgtg
atgacccagtctccagactccctggctgtgtctctgggcgagagggccaccatcaactgcagggccagcaagagtgttagc
accagcggctacagctacatgcactggtaccagcagaaaccaggacagcctcctaagctgctcatttacctggtgtctaac
ctggaatccggggtccctgaccgattcagtggcagcgggtctgggacagatttcactctcaccatcagcagcctgcaggct
gaagatgtggcagtttattactgtcaacacattcgggaactgaccaggagtgaattcggcggagggaccaaggtggagatc
aaacgaactgagcccaaatcttgtgacaaaactcacacatgcccaccgtgcccagcacctgaactcctggggggaccgtca
gtcttcctcttccccccaaaacccaaggacaccctcatgatctcccggacccctgaggtcacatgcgtggtggtggacgtg
agccacgaagaccctgaggtcaagttcaactggtacgtggacggcgtggaggtgcataatgccaagacaaagccgcgggag
gagcagtacaacagcacgtaccgtgtggtcagcgtcctcaccgtcctgcaccaggactggctgaatggcaaggagtacaag
tgcaaggtctccaacaaagccctcccagcccccatcgagaaaaccatctccaaagccaaagggcagccccgagaaccacag
gtgtacaccctgcccccatcccgggaggagatgaccaagaaccaggtcagcctgacctgcctggtcaaaggcttctatccc
agcgacatcgccgtggagtgggagagcaatgggcagccggagaacaactacaagaccacgcctcccgtgctggactccgac
ggctccttcttcctctacagcaagctcaccgtggacaagagcaggtggcagcaggggaacgtcttctcatgctccgtgatg
catgaggctctgcacaaccactacacgcagaagagcctctccctgtctccgggtaaatgataa
(amino acids)
(SEQ ID NO: 269)
METDTLLLWVLLLWVPGSTGDAAQPAEVQLVESGGGLVKPGGSLRLSCAASGFTFSGYAMSWVRQAPGKGLEWVSTISSG
GTYIYYPDSVKGRFTISRDNAKNSLYLQMNSLRAEDTAVYYCARLGGDNYYEYWGKGTTVTVSSGGGGSGGGGSGGGGSD
IVMTQSPDSLAVSLGERATINCRASKSVSTSGYSYMHWYQQKPGQPPKLLIYLVSNLESGVPDRFSGSGSGTDFTLTISS
LQAEDVAVYYCQHIRELTRSEFGGGTKVEIKRTEPKSCDKTHTCPPCPAPELLGGPSVFLFPPKPKDTLMISRTPEVTCV
VVDVSHEDPEVKFNWYVDGVEVHNAKTKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFLYSKLTVDKSRWQQGNV
FSCSVMHEALHNHYTQKSLSLSPGK**
C8 scFV FC 1 gBLOCk sequence:
(DNA)
(SEQ ID NO: 270)
tgctctgggttccaggttccactggtgacgcggcccagccggccgaggtgcagctggtggagtctgggggaggcctggtca
agcctggggggtccctgagactctcctgtgcagcctctggattcaccttcagtggctatgccatgagctgggtccgccagg
ctccagggaaggggctggagtgggtctcaaccattagtagtggcggaacctacatatactaccctgactcagtgaagggcc
gattcaccatctccagagacaacgccaagaactcactgtatctgcaaatgaacagcctgagagccgaggacacggccgtgt
attactgtgcgagactgggcggcgataactattatgaatattggggcaaagggaccacggtcaccgtctcctccggcggtg
gcggatccggcggtggcggatccggcggtggcggatccgacatcgtgatgacccagtctccagactccctgg
C8 scFV FC2 gBLOCk sequence:
(DNA)
(SEQ ID NO: 271)
catcgtgatgacccagtctccagactccctggctgtgtctctgggcgagagggccaccatcaactgcagggccagcaagag
tgttagcaccagcggctacagctacatgcactggtaccagcagaaaccaggacagcctcctaagctgctcatttacctggt
gtctaacctggaatccggggtccctgaccgattcagtggcagcgggtctgggacagatttcactctcaccatcagcagcct
gcaggctgaagatgtggcagtttattactgtcaacacattcgggaactgaccaggagtgaattcggcggagggaccaaggt
ggagatcaaacgaactgagcccaaatcttgtgacaaaactcacacatgcccaccgtgcccagcacctgaactcctgggggg
accgtcagtcttcctcttccccccaaaacccaaggacaccctcatgatctcccggacccctgaggtcacatgcgtggtggt
ggacgtgagccacgaagaccctgaggtcaagttcaactggtacgtggacggcgtggaggtgcataatgccaagacaaagcc
gcgggaggagcagtacaacagcacgtaccgtgtggtcagc
Human IgG1 Fc sequence:
(DNA)
(SEQ ID NO: 272)
gagcccaaatcttgtgacaaaactcacacatgcccaccgtgcccagcacctgaactcctggggggaccgtcagtcttcctc
ttccccccaaaacccaaggacaccctcatgatctcccggacccctgaggtcacatgcgtggtggtggacgtgagccacgaa
gaccctgaggtcaagttcaactggtacgtggacggcgtggaggtgcataatgccaagacaaagccgcgggaggagcagtac
aacagcacgtaccgtgtggtcagcgtcctcaccgtcctgcaccaggactggctgaatggcaaggagtacaagtgcaaggtc
tccaacaaagccctcccagcccccatcgagaaaaccatctccaaagccaaagggcagccccgagaaccacaggtgtacacc
ctgcccccatcccgggaggagatgaccaagaaccaggtcagcctgacctgcctggtcaaaggcttctatcccagcgacatc
gccgtggagtgggagagcaatgggcagccggagaacaactacaagaccacgcctcccgtgctggactccgacggctccttc
ttcctctacagcaagctcaccgtggacaagagcaggtggcagcaggggaacgtcttctcatgctccgtgatgcatgaggct
ctgcacaaccactacacgcagaagagcctctccctgtctccgggtaaatgataa
(amino acids)
(SEQ ID NO: 273)
EPKSCDKTHTCPPCPAPELLGGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVFSCSVMHEALHNHYTQKSLSLSPGK**
Human IgG1 CH2-CH3 domain sequence:
(DNA)
(SEQ ID NO: 274)
ccgtgcccagcacctgaactcctggggggaccgtcagtcttcctcttccccccaaaacccaaggacaccctcatgatctcc
cggacccctgaggtcacatgcgtggtggtggacgtgagccacgaagaccctgaggtcaagttcaactggtacgtggacggc
gtggaggtgcataatgccaagacaaagccgcgggaggagcagtacaacagcacgtaccgtgtggtcagcgtcctcaccgtc
ctgcaccaggactggctgaatggcaaggagtacaagtgcaaggtctccaacaaagccctcccagcccccatcgagaaaacc
atctccaaagccaaagggcagccccgagaaccacaggtgtacaccctgcccccatcccgggaggagatgaccaagaaccag
gtcagcctgacctgcctggtcaaaggcttctatcccagcgacatcgccgtggagtgggagagcaatgggcagccggagaac
aactacaagaccacgcctcccgtgctggactccgacggctccttcttcctctacagcaagctcaccgtggacaagagcagg
tggcagcaggggaacgtcttctcatgctccgtgatgcatgaggctctgcacaaccactacacgcagaagagcctctccctg
tctccgggtaaatgataa
(amino acids)
(SEQ ID NO: 275)
PCPAPELLGGPSVFLFPPKPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTKNQVSLTCLVKGFYPSDIAVEWESNGQP
ENNYKTTPPVLDSDGSFFLYSKLTVDKSRWQQGNVFSCSVMHEALHNHYTQKSLSLSPGK**
Human IgG1 CH3 domain sequence:
(DNA)
(SEQ ID NO: 276)
gggcagccccgagaaccacaggtgtacaccctgcccccatcccgggaggagatgaccaagaaccaggtcagcctgacctgc
ctggtcaaaggcttctatcccagcgacatcgccgtggagtgggagagcaatgggcagccggagaacaactacaagaccacg
cctcccgtgctggactccgacggctccttcttcctctacagcaagctcaccgtggacaagagcaggtggcagcaggggaac
gtcttctcatgctccgtgatgcatgaggctctgcacaaccactacacgcagaagagcctctccctgtctccgggtaaatga
taa
(amino acids)
(SEQ ID NO: 277)
GQPREPQVYTLPPSREEMTKNQVSLTCLVKGFYPSDIAVEWESNGQPENNYKTTPPVLDSDGSFFLYSKLTVDKSRWQQG
NVFSCSVMHEALHNHYTQKSLSLSPGK**
Human IgG1 Fc Y407R sequence:
(DNA)
(SEQ ID NO: 278)
gagcccaaatcttgtgacaaaactcacacatgcccaccgtgcccagcacctgaactcctggggggaccgtcagtcttcctc
ttccccccaaaacccaaggacaccctcatgatctcccggacccctgaggtcacatgcgtggtggtggacgtgagccacgaa
gaccctgaggtcaagttcaactggtacgtggacggcgtggaggtgcataatgccaagacaaagccgcgggaggagcagtac
aacagcacgtaccgtgtggtcagcgtcctcaccgtcctgcaccaggactggctgaatggcaaggagtacaagtgcaaggtc
tccaacaaagccctcccagcccccatcgagaaaaccatctccaaagccaaagggcagccccgagaaccacaggtgtacacc
ctgcccccatcccgggaggagatgaccaagaaccaggtcagcctgacctgcctggtcaaaggcttctatcccagcgacatc
gccgtggagtgggagagcaatgggcagccggagaacaactacaagaccacgcctcccgtgctggactccgacggctccttc
ttcctcaggagcaagctcaccgtggacaagagcaggtggcagcaggggaacgtcttctcatgctccgtgatgcatgaggct
ctgcacaaccactacacgcagaagagcctctccctgtctccgggtaaatgataa
(amino acids)
(SEQ ID NO: 279)
EPKSCDKTHTCPPCPAPELLGGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSREEMTKNQVSLTCLVKGFYPS
DIAVEWESNGQPENNYKTTPPVLDSDGSFFLRSKLTVDKSRWQQGNVFSCSVMHEALHNHYTQKSLSLSPGK**
Human IgG1 Fc F405Q sequence:
(DNA)
(SEQ ID NO: 280)
gagcccaaatcttgtgacaaaactcacacatgcccaccgtgcccagcacctgaactcctggggggaccgtcagtcttcctc
ttccccccaaaacccaaggacaccctcatgatctcccggacccctgaggtcacatgcgtggtggtggacgtgagccacgaa
gaccctgaggtcaagttcaactggtacgtggacggcgtggaggtgcataatgccaagacaaagccgcgggaggagcagtac
aacagcacgtaccgtgtggtcagcgtcctcaccgtcctgcaccaggactggctgaatggcaaggagtacaagtgcaaggtc
tccaacaaagccctcccagcccccatcgagaaaaccatctccaaagccaaagggcagccccgagaaccacaggtgtacacc
ctgcccccatcccgggaggagatgaccaagaaccaggtcagcctgacctgcctggtcaaaggcttctatcccagcgacatc
gccgtggagtgggagagcaatgggcagccggagaacaactacaagaccacgcctcccgtgctggactccgacggctccttc
cagctctacagcaagctcaccgtggacaagagcaggtggcagcaggggaacgtcttctcatgctccgtgatgcatgaggct
ctgcacaaccactacacgcagaagagcctctccctgtctccgggtaaatgataa
(amino acids)
(SEQ ID NO: 281)
EPKSCDKTHTCPPCPAPELLGGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSREEMTKNQVSLTCLVKGFYPS
DIAVEWESNGQPENNYKTTPPVLDSDGSFQLYSKLTVDKSRWQQGNVFSCSVMHEALHNHYTQKSLSLSPGK**
Human IgG1 Fc T394D sequence:
(DNA)
(SEQ ID NO: 282)
gagcccaaatcttgtgacaaaactcacacatgcccaccgtgcccagcacctgaactcctggggggaccgtcagtcttcctc
ttccccccaaaacccaaggacaccctcatgatctcccggacccctgaggtcacatgcgtggtggtggacgtgagccacgaa
gaccctgaggtcaagttcaactggtacgtggacggcgtggaggtgcataatgccaagacaaagccgcgggaggagcagtac
aacagcacgtaccgtgtggtcagcgtcctcaccgtcctgcaccaggactggctgaatggcaaggagtacaagtgcaaggtc
tccaacaaagccctcccagcccccatcgagaaaaccatctccaaagccaaagggcagccccgagaaccacaggtgtacacc
ctgcccccatcccgggaggagatgaccaagaaccaggtcagcctgacctgcctggtcaaaggcttctatcccagcgacatc
gccgtggagtgggagagcaatgggcagccggagaacaactacaagaccgaccctcccgtgctggactccgacggctccttc
ttcctctacagcaagctcaccgtggacaagagcaggtggcagcaggggaacgtcttctcatgctccgtgatgcatgaggct
ctgcacaaccactacacgcagaagagcctctccctgtctccgggtaaatgataa
(amino acids)
(SEQ ID NO: 283)
EPKSCDKTHTCPPCPAPELLGGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSREEMTKNQVSLTCLVKGFYPS
DIAVEWESNGQPENNYKTDPPVLDSDGSFFLYSKLTVDKSRWQQGNVFSCSVMHEALHNHYTQKSLSLSPGK**
Human IgG1 Fc T366W/L368W sequence:
(DNA)
(SEQ ID NO: 284)
gagcccaaatcttgtgacaaaactcacacatgcccaccgtgcccagcacctgaactcctggggggaccgtcagtcttcctc
ttccccccaaaacccaaggacaccctcatgatctcccggacccctgaggtcacatgcgtggtggtggacgtgagccacgaa
gaccctgaggtcaagttcaactggtacgtggacggcgtggaggtgcataatgccaagacaaagccgcgggaggagcagtac
aacagcacgtaccgtgtggtcagcgtcctcaccgtcctgcaccaggactggctgaatggcaaggagtacaagtgcaaggtc
tccaacaaagccctcccagcccccatcgagaaaaccatctccaaagccaaagggcagccccgagaaccacaggtgtacacc
ctgcccccatcccgggaggagatgaccaagaaccaggtcagcctgtggtgctgggtcaaaggcttctatcccagcgacatc
gccgtggagtgggagagcaatgggcagccggagaacaactacaagaccacgcctcccgtgctggactccgacggctccttc
ttcctctacagcaagctcaccgtggacaagagcaggtggcagcaggggaacgtcttctcatgctccgtgatgcatgaggct
ctgcacaaccactacacgcagaagagcctctccctgtctccgggtaaatgataa
(amino acids)
(SEQ ID NO: 285)
EPKSCDKTHTCPPCPAPELLGGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSREEMTKNQVSLWCWVKGFYPS
DIAVEWESNGQPENNYKTTPPVLDSDGSFFLYSKLTVDKSRWQQGNVFSCSVMHEALHNHYTQKSLSLSPGK**
Human IgG1 Fc T364R/L368R sequence:
(DNA)
(SEQ ID NO: 286)
gagcccaaatcttgtgacaaaactcacacatgcccaccgtgcccagcacctgaactcctggggggaccgtcagtcttcctc
ttccccccaaaacccaaggacaccctcatgatctcccggacccctgaggtcacatgcgtggtggtggacgtgagccacgaa
gaccctgaggtcaagttcaactggtacgtggacggcgtggaggtgcataatgccaagacaaagccgcgggaggagcagtac
aacagcacgtaccgtgtggtcagcgtcctcaccgtcctgcaccaggactggctgaatggcaaggagtacaagtgcaaggtc
tccaacaaagccctcccagcccccatcgagaaaaccatctccaaagccaaagggcagccccgagaaccacaggtgtacacc
ctgcccccatcccgggaggagatgaccaagaaccaggtcaggctgacctgcagggtcaaaggcttctatcccagcgacatc
gccgtggagtgggagagcaatgggcagccggagaacaactacaagaccacgcctcccgtgctggactccgacggctccttc
ttcctctacagcaagctcaccgtggacaagagcaggtggcagcaggggaacgtcttctcatgctccgtgatgcatgaggct
ctgcacaaccactacacgcagaagagcctctccctgtctccgggtaaatgataa
(amino acids)
(SEQ ID NO: 287)
EPKSCDKTHTCPPCPAPELLGGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSREEMTKNQVRLTCRVKGFYPS
DIAVEWESNGQPENNYKTTPPVLDSDGSFFLYSKLTVDKSRWQQGNVFSCSVMHEALHNHYTQKSLSLSPGK**
Human IgG1 Fc hingeless sequence:
(DNA)
(SEQ ID NO: 288)
gcacctgaactcctggggggaccgtcagtcttcctcttccccccaaaacccaaggacaccctcatgatctcccggacccct
gaggtcacatgcgtggtggtggacgtgagccacgaagaccctgaggtcaagttcaactggtacgtggacggcgtggaggtg
cataatgccaagacaaagccgcgggaggagcagtacaacagcacgtaccgtgtggtcagcgtcctcaccgtcctgcaccag
gactggctgaatggcaaggagtacaagtgcaaggtctccaacaaagccctcccagcccccatcgagaaaaccatctccaaa
gccaaagggcagccccgagaaccacaggtgtacaccctgcccccatcccgggaggagatgaccaagaaccaggtcagcctg
acctgcctggtcaaaggcttctatcccagcgacatcgccgtggagtgggagagcaatgggcagccggagaacaactacaag
accacgcctcccgtgctggactccgacggctccttcttcctctacagcaagctcaccgtggacaagagcaggtggcagcag
gggaacgtcttctcatgctccgtgatgcatgaggctctgcacaaccactacacgcagaagagcctctccctgtctccgggt
aaatgataa
(amino acids)
(SEQ ID NO: 289)
APELLGGPSVFLFPPKPKDTLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKSLSLSPGK**
Human IgG1 G237A FC sequence:
(DNA)
(SEQ ID NO: 290)
gagcccaaatcttgtgacaaaactcacacatgcccaccgtgcccagcacctgaactcctgggggccccgtcagtcttcctc
ttccccccaaaacccaaggacaccctcatgatctcccggacccctgaggtcacatgcgtggtggtggacgtgagccacgaa
gaccctgaggtcaagttcaactggtacgtggacggcgtggaggtgcataatgccaagacaaagccgcgggaggagcagtac
aacagcacgtaccgtgtggtcagcgtcctcaccgtcctgcaccaggactggctgaatggcaaggagtacaagtgcaaggtc
tccaacaaagccctcccagcccccatcgagaaaaccatctccaaagccaaagggcagccccgagaaccacaggtgtacacc
ctgcccccatcccgggaggagatgaccaagaaccaggtcagcctgacctgcctggtcaaaggcttctatcccagcgacatc
gccgtggagtgggagagcaatgggcagccggagaacaactacaagaccacgcctcccgtgctggactccgacggctccttc
ttcctctacagcaagctcaccgtggacaagagcaggtggcagcaggggaacgtcttctcatgctccgtgatgcatgaggct
ctgcacaaccactacacgcagaagagcctctccctgtctccgggtaaa
(amino acids)
(SEQ ID NO: 291)
EPKSCDKTHTCPPCPAPELLGAPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSVMHEALHNHYTQKSLSLSPGK
Human IgG1 L234A/L235A FC sequence:
(DNA)
(SEQ ID NO: 292)
gagcccaaatcttgtgacaaaactcacacatgcccaccgtgcccagcacctgaagccgccgggggaccgtcagtcttcctc
ttccccccaaaacccaaggacaccctcatgatctcccggacccctgaggtcacatgcgtggtggtggacgtgagccacgaa
gaccctgaggtcaagttcaactggtacgtggacggcgtggaggtgcataatgccaagacaaagccgcgggaggagcagtac
aacagcacgtaccgtgtggtcagcgtcctcaccgtcctgcaccaggactggctgaatggcaaggagtacaagtgcaaggtc
tccaacaaagccctcccagcccccatcgagaaaaccatctccaaagccaaagggcagccccgagaaccacaggtgtacacc
ctgcccccatcccgggaggagatgaccaagaaccaggtcagcctgacctgcctggtcaaaggcttctatcccagcgacatc
gccgtggagtgggagagcaatgggcagccggagaacaactacaagaccacgcctcccgtgctggactccgacggctccttc
ttcctctacagcaagctcaccgtggacaagagcaggtggcagcaggggaacgtcttctcatgctccgtgatgcatgaggct
ctgcacaaccactacacgcagaagagcctctccctgtctccgggtaaa
(amino acids)
(SEQ ID NO: 293)
EPKSCDKTHTCPPCPAPEAAGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSVMHEALHNHYTQKSLSLSPGK
CAR-T E6 CD8/CD8/CD3z sequence:
N-CD8ls-huMNE6scFv-CD8ecd fragment- CD8 transmembrane- CD3zeta-C
(DNA)
(SEQ ID NO: 294)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggggggggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaaacgacaaccccggccccc
agaccaccaacgccagcccccaccatcgccagccaacccctgtctctgagaccagaagcctgtaggcctgccgccggtgga
gctgtgcacacaagaggactggatttcgcctgtgatatctacatttgggccccgctcgcaggcacatgtggagtgctcctc
ctctccctggtgattaccctgtactgccgcgttaagttctcccgatcagccgacgcgcctgcttacaagcagggccagaac
caactgtacaacgagctgaatctcggtagacgggaagagtacgacgtgttggacaaacggagaggccgcgacccagaaatg
ggcggcaagcctcgcaggaaaaacccccaggagggactgtacaatgagttgcagaaagataagatggcagaagcttatagc
gagatcggaatgaagggggaaaggagacgagggaaaggacacgacggcctttatcagggcctgtccacagcaacaaaagat
acgtatgacgccctccatatgcaggcacttccaccacggtgataa
(amino acids)
(SEQ ID NO: 295)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKTTTPAPRPPTPAPTIASQPLSLRPEACRPAAGGAVHTRGLDFACDIYIWAPLAGTC
GVLLLSLVITLYCRVKFSRSADAPAYKQGQNQLYNELNLGRREEYDVLDKRRGRDPEMGGKPRRKNPQEGLYNELQKDKM
AEAYSEIGMKGERRRGKGHDGLYQGLSTATKDTYDALHMQALPPR**
**
CAR-T E6 CD3z gBLOCK sequence:
(DNA)
(SEQ ID NO: 296)
tggagctgtgcacacaagaggactggatttcgcctgtgatatctacatttgggccccgctcgcaggcacatgtggagtgct
cctcctctccctggtgattaccctgtactgccgcgttaagttctcccgatcagccgacgcgcctgcttacaagcagggcca
gaaccaactgtacaacgagctgaatctcggtagacgggaagagtacgacgtgttggacaaacggagaggccgcgacccaga
aatgggcggcaagcctcgcaggaaaaacccccaggagggactgtacaatgagttgcagaaagataagatggcagaagctta
tagcgagatcggaatgaagggggaaaggagacgagggaaaggacacgacggcctttatcagggcctgtccacagcaacaaa
agatacgtatgacgccctccatatgcaggcacttccaccacggtgataagtttaaacccgctgatcagcctcgactgtgc
CAR-T E6 CD8/CD8/CD28/CD3z sequence:
N-CD8ls-huMNE6scFv-CD8ecd fragment- CD8 transmembrane- CD28- CD3zeta-C
(DNA)
(SEQ ID NO: 297)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggggggggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaaacgacaaccccggccccc
agaccaccaacgccagcccccaccatcgccagccaacccctgtctctgagaccagaagcctgtaggcctgccgccggtgga
gctgtgcacacaagaggactggatttcgcctgtgatatctacatttgggccccgctcgcaggcacatgtggagtgctcctc
ctctccctggtgattaccctgtactgcagaagcaagcggtctcggctcctgcattctgattacatgaacatgaccccaaga
agaccaggccccaccaggaaacattaccagccctacgctccgccacgcgacttcgctgcctaccggtcccgcgttaagttc
tcccgatcagccgacgcgcctgcttacaagcagggccagaaccaactgtacaacgagctgaatctcggtagacgggaagag
tacgacgtgttggacaaacggagaggccgcgacccagaaatgggcggcaagcctcgcaggaaaaacccccaggagggactg
tacaatgagttgcagaaagataagatggcagaagcttatagcgagatcggaatgaagggggaaaggagacgagggaaagga
cacgacggcctttatcagggcctgtccacagcaacaaaagatacgtatgacgccctccatatgcaggcacttccaccacgg
tgataa
(amino acids)
(SEQ ID NO: 298)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKTTTPAPRPPTPAPTIASQPLSLRPEACRPAAGGAVHTRGLDFACDIYIWAPLAGTC
GVLLLSLVITLYCRSKRSRLLHSDYMNMTPRRPGPTRKHYQPYAPPRDFAAYRSRVKFSRSADAPAYKQGQNQLYNELNL
GRREEYDVLDKRRGRDPEMGGKPRRKNPQEGLYNELQKDKMAEAYSEIGMKGERRRGKGHDGLYQGLSTATKDTYDALHM
QALPPR**
CAR-T E6 CD28/CD3z g BLOCK sequence:
(DNA)
(SEQ ID NO: 299)
tggagctgtgcacacaagaggactggatttcgcctgtgatatctacatttgggccccgctcgcaggcacatgtggagtgct
cctcctctccctggtgattaccctgtactgcagaagcaagcggtctcggctcctgcattctgattacatgaacatgacccc
aagaagaccaggccccaccaggaaacattaccagccctacgctccgccacgcgacttcgctgcctaccggtcccgcgttaa
gttctcccgatcagccgacgcgcctgcttacaagcagggccagaaccaactgtacaacgagctgaatctcggtagacggga
agagtacgacgtgttggacaaacggagaggccgcgacccagaaatgggcggcaagcctcgcaggaaaaacccccaggaggg
actgtacaatgagttgcagaaagataagatggcagaagcttatagcgagatcggaatgaagggggaaaggagacgagggaa
aggacacgacggcctttatcagggcctgtccacagcaacaaaagatacgtatgacgccctccatatgcaggcacttccacc
acggtgataagtttaaacccgctgatcagcctcgactgtgc
CAR-T E6 CD8/CD8/4-1BB/CD3z sequence:
N-CD8ls-huMNE6scFv-CD8ecd fragment- CD8 transmembrane- 4-1BB- CD3zeta-C
(DNA)
(SEQ ID NO: 300)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggggggggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaaacgacaaccccggccccc
agaccaccaacgccagcccccaccatcgccagccaacccctgtctctgagaccagaagcctgtaggcctgccgccggtgga
gctgtgcacacaagaggactggatttcgcctgtgatatctacatttgggccccgctcgcaggcacatgtggagtgctcctc
ctctccctggtgattaccctgtactgcaaaaggggccgcaaaaaactcctttacatttttaagcagccttttatgaggcca
gtacagacgactcaagaggaagacgggtgctcatgccgctttcctgaggaggaggaaggagggtgcgaactgcgcgttaag
ttctcccgatcagccgacgcgcctgcttacaagcagggccagaaccaactgtacaacgagctgaatctcggtagacgggaa
gagtacgacgtgttggacaaacggagaggccgcgacccagaaatgggcggcaagcctcgcaggaaaaacccccaggaggga
ctgtacaatgagttgcagaaagataagatggcagaagcttatagcgagatcggaatgaagggggaaaggagacgagggaaa
ggacacgacggcctttatcagggcctgtccacagcaacaaaagatacgtatgacgccctccatatgcaggcacttccacca
cggtgataa
(amino acids)
(SEQ ID NO: 301)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKTTTPAPRPPTPAPTIASQPLSLRPEACRPAAGGAVHTRGLDFACDIYIWAPLAGTC
GVLLLSLVITLYCKRGRKKLLYIFKQPFMRPVQTTQEEDGCSCRFPEEEEGGCELRVKFSRSADAPAYKQGQNQLYNELN
LGRREEYDVLDKRRGRDPEMGGKPRRKNPQEGLYNELQKDKMAEAYSEIGMKGERRRGKGHDGLYQGLSTATKDTYDALH
MQALPPR**
CAR-T E6 4-1BB/CD3z gBLOCK sequence:
(DNA)
(SEQ ID NO: 302)
tggagctgtgcacacaagaggactggatttcgcctgtgatatctacatttgggccccgctcgcaggcacatgtggagtgct
cctcctctccctggtgattaccctgtactgcaaaaggggccgcaaaaaactcctttacatttttaagcagccttttatgag
gccagtacagacgactcaagaggaagacgggtgctcatgccgctttcctgaggaggaggaaggagggtgcgaactgcgcgt
taagttctcccgatcagccgacgcgcctgcttacaagcagggccagaaccaactgtacaacgagctgaatctcggtagacg
ggaagagtacgacgtgttggacaaacggagaggccgcgacccagaaatgggcggcaagcctcgcaggaaaaacccccagga
gggactgtacaatgagttgcagaaagataagatggcagaagcttatagcgagatcggaatgaagggggaaaggagacgagg
gaaaggacacgacggcctttatcagggcctgtccacagcaacaaaagatacgtatgacgccctccatatgcaggcacttcc
accacggtgataagtttaaacccgctgatcagcctcgactgtgc
CAR-T E6 CD8/CD8/CD28/4-1BB/CD3z sequence:
N-CD8ls-huMNE6scFv-CD8ecd fragment- CD8 transmembrane- CD28- 4-1BB- CD3zeta-C
(DNA)
(SEQ ID NO: 303)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggggggggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaaacgacaaccccggccccc
agaccaccaacgccagcccccaccatcgccagccaacccctgtctctgagaccagaagcctgtaggcctgccgccggtgga
gctgtgcacacaagaggactggatttcgcctgtgatatctacatttgggccccgctcgcaggcacatgtggagtgctcctc
ctctccctggtgattaccctgtactgcagaagcaagcggtctcggctcctgcattctgattacatgaacatgaccccaaga
agaccaggccccaccaggaaacattaccagccctacgctccgccacgcgacttcgctgcctaccggtccaaaaggggccgc
aaaaaactcctttacatttttaagcagccttttatgaggccagtacagacgactcaagaggaagacgggtgctcatgccgc
tttcctgaggaggaggaaggagggtgcgaactgcgcgttaagttctcccgatcagccgacgcgcctgcttacaagcagggc
cagaaccaactgtacaacgagctgaatctcggtagacgggaagagtacgacgtgttggacaaacggagaggccgcgaccca
gaaatgggcggcaagcctcgcaggaaaaacccccaggagggactgtacaatgagttgcagaaagataagatggcagaagct
tatagcgagatcggaatgaagggggaaaggagacgagggaaaggacacgacggcctttatcagggcctgtccacagcaaca
aaagatacgtatgacgccctccatatgcaggcacttccaccacggtgataa
(amino acids)
(SEQ ID NO: 304)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKTTTPAPRPPTPAPTIASQPLSLRPEACRPAAGGAVHTRGLDFACDIYIWAPLAGTC
GVLLLSLVITLYCRSKRSRLLHSDYMNMTPRRPGPTRKHYQPYAPPRDFAAYRSKRGRKKLLYIFKQPFMRPVQTTQEED
GCSCRFPEEEEGGCELRVKFSRSADAPAYKQGQNQLYNELNLGRREEYDVLDKRRGRDPEMGGKPRRKNPQEGLYNELQK
DKMAEAYSEIGMKGERRRGKGHDGLYQGLSTATKDTYDALHMQALPPR**
CAR-T E6 CD28/4-1BB/CD3z gBLOCK sequence:
(DNA)
(SEQ ID NO: 305)
atagggagacccaagctggctagttaagcttggtaccgagggccaccatggccctgcccgtgaccgctttgctgctccccc
tggcgctgctgctgcacgccgccaggccagaggtccagctggttgagagtggcggtgggctggttaagcctggcggctccc
tgcggctgagctgcgccgcgagtggatttactttcagccgatatgggatgagttgggtgcggcaagctcccgggaagaggc
tggaatgggtctcaacaatctccggggggggcacttacatctattaccccgactcagtcaaggggagatttaccatttcac
gagacaacgctaagaataccctgtatttgcagatgaattctctgagagcagaggacacagctgtttactattgtacccgcg
acaactatggcaggaactacgactacggtatggactattggggacaagggacattggttacagtgagcagtggcggcgggg
gcagcggaggaggaggcagcggtggggggggcagcgagatagtgctcacgcagtcacccgcgactctcagtctctcacctg
gggaacgagctaccctgacgtgctctgctacctcctcagtgtcatatattcactggtatcagcaacggcccgggcagtccc
ctagattgctcatttatagtacctctaatctggcctcaggtatccctgcacgattttctggatctggttcaggttctgatt
acaccctcactatctctagcctggagcctgaagactttgccgtttattactgccagcagaggtctagctccccattcacct
ttgggagtgggaccaaggttgaaattaaaacgacaaccccggcccccagaccaccaacgccagcccccaccatcgccagcc
aacccctgtctctgagaccagaagcctgtaggcctgccgccggtggagctgtgcacacaagaggactggatttcgcctgtg
atatctacatttgggccccgctcgcaggcacatgtggagtgctcctcctctccctggtgattaccctgtactgcagaagca
agcggtctcggctcctgcattctgattacatgaacatgaccccaagaagaccaggccccaccaggaaacattaccagccct
acgctccgccacgcgacttcgctgcctaccggtccaaaaggggccgcaaaaaactcctttacatttttaagcagcctttta
tgaggccagtacagacgactcaagaggaagacgggtgctcatgccgctttcctgaggaggaggaaggagggtgcgaactgc
gcgttaagttctcccgatcagccgacgcgcctgcttacaagcagggccagaaccaactgtacaacgagctgaatctcggta
gacgggaagagtacgacgtgttggacaaacggagaggccgcgacccagaaatgggcggcaagcctcgcaggaaaaaccccc
aggagggactgtacaatgagttgcagaaagataagatggcagaagcttatagcgagatcggaatgaagggggaaaggagac
gagggaaaggacacgacggcctttatcagggcctgtccacagcaacaaaagatacgtatgacgccctccatatgcaggcac
ttccaccacggtgataagtttaaacccgctgatcagcctcgactgtgc
CAR-T C2 CD8/CD8/CD28/4-1BB/CD3z sequence:
N-CD8ls-huMNC2scFv-CD8ecd fragment- CD8 transmembrane- CD28- 4-1BB- CD3zeta-C
(DNA)
(SEQ ID NO: 306)
atggccttgccagtgacggccctgctgctgccattggctcttctgttgcacgctgccaggcctgaagtgcagctcgtagag
agtggcgggggactggtgaagcccggtggaagcctcagactcagttgcgccgcctcaggtttcactttttcaggttacgcc
atgtcctgggtaagacaggcaccggggaaaggactcgagtgggtgtctactatcagctcaggaggcacttatatatattat
cctgactctgtaaaaggccgatttacgatttctcgcgacaatgcaaagaactccctctacctccaaatgaacagtcttagg
gcagaagacactgctgtatactattgtgcacgcctcggcggcgacaactactacgagtactttgacgtgtgggggaaaggg
actaccgtgacagtttcaagcggaggaggtggctcaggtggaggcgggtcaggggggggaggaagtgatattgtgctcaca
caatccccagcctccctggctgtgtctcccggccaacgcgctacaattacatgtcgggcctccaaaagcgtgagcaccagc
ggctacagctacatgcactggtatcaacagaaaccaggacaaccccccaaactgttgatttatctcgcttcaaacttggag
tccggcgtgcctgcgcgcttttcagggagtgggagcggcacagattttacgctgactatcaaccccgtagaagcaaacgat
acagcgaattattattgtcaacattcccgggaactcccctttacgttcggcgggggcacaaaggtcgaaattaagagaacc
acgacaaccccggcccccagaccaccaacgccagcccccaccatcgccagccaacccctgtctctgagaccagaagcctgt
aggcctgccgccggtggagctgtgcacacaagaggactggatttcgcctgtgatatctacatttgggccccgctcgcaggc
acatgtggagtgctcctcctctccctggtgattaccctgtactgcagaagcaagcggtctcggctcctgcattctgattac
atgaacatgaccccaagaagaccaggccccaccaggaaacattaccagccctacgctccgccacgcgacttcgctgcctac
cggtccaaaaggggccgcaaaaaactcctttacatttttaagcagccttttatgaggccagtacagacgactcaagaggaa
gacgggtgctcatgccgctttcctgaggaggaggaaggagggtgcgaactgcgcgttaagttctcccgatcagccgacgcg
cctgcttacaagcagggccagaaccaactgtacaacgagctgaatctcggtagacgggaagagtacgacgtgttggacaaa
cggagaggccgcgacccagaaatgggcggcaagcctcgcaggaaaaacccccaggagggactgtacaatgagttgcagaaa
gataagatggcagaagcttatagcgagatcggaatgaagggggaaaggagacgagggaaaggacacgacggcctttatcag
ggcctgtccacagcaacaaaagatacgtatgacgccctccatatgcaggcacttccaccacggtgataa
(amino acids)
(SEQ ID NO: 307)
EVQLVESGGGLVKPGGSLRLSCAASGFTFSGYAMSWVRQAPGKGLEWVSTISSGGTYIYYPDSVKGRFTISRDNAKNSLYL
QMNSLRAEDTAVYYCARLGGDNYYEYFDVWGKGTTVTVSSGGGGSGGGGSGGGGSDIVLTQSPASLAVSPGQRATITCRAS
KSVSTSGYSYMHWYQQKPGQPPKLLIYLASNLESGVPARFSGSGSGTDFTLTINPVEANDTANYYCQHSRELPFTFGGGTK
VEIKRTTTTPAPRPPTPAPTIASQPLSLRPEACRPAAGGAVHTRGLDFACDIYIWAPLAGTCGVLLLSLVITLYCRSKRSR
LLHSDYMNMTPRRPGPTRKHYQPYAPPRDFAAYRSKRGRKKLLYIFKQPFMRPVQTTQEEDGCSCRFPEEEEGGCELRVKF
SRSADAPAYKQGQNQLYNELNLGRREEYDVLDKRRGRDPEMGGKPRRKNPQEGLYNELQKDKMAEAYSEIGMKGERRRGKG
HDGLYQGLSTATKDTYDALHMQALPPR**
CAR-T C2-1 gBLOCK sequence:
(DNA)
(SEQ ID NO: 308)
atagggagacccaagctggctagttaagcttggtaccgagggccaccatggccttgccagtgacggccctgctgctgccat
tggctcttctgttgcacgctgccaggcctgaagtgcagctcgtagagagtggcgggggactggtgaagcccggtggaagcc
tcagactcagttgcgccgcctcaggtttcactttttcaggttacgccatgtcctgggtaagacaggcaccggggaaaggac
tcgagtgggtgtctactatcagctcaggaggcacttatatatattatcctgactctgtaaaaggccgatttacgatttctc
gcgacaatgcaaagaactccctctacctccaaatgaacagtcttagggcagaagacactgctgtatactattgtgcacgcc
tcggcggcgacaactactacgagtactttgacgtgtgggggaaagggactaccgtgacagtttcaagcggaggaggtggct
caggtggaggcgggtcaggggggggaggaagtgatattgtgctcacacaatccccagcctccctggc
CAR-T C2-2 gBLOCK sequence:
(DNA)
(SEQ ID NO: 309)
aagtgatattgtgctcacacaatccccagcctccctggctgtgtctcccggccaacgcgctacaattacatgtcgggcctc
caaaagcgtgagcaccagcggctacagctacatgcactggtatcaacagaaaccaggacaaccccccaaactgttgattta
tctcgcttcaaacttggagtccggcgtgcctgcgcgcttttcagggagtgggagcggcacagattttacgctgactatcaa
ccccgtagaagcaaacgatacagcgaattattattgtcaacattcccgggaactcccctttacgttcggcgggggcacaaa
ggtcgaaattaagagaaccacgacaaccccggcccccagaccaccaacgccagcccccaccatcgccagccaacccctgtc
tctgagaccagaagcctgtaggcctgccgccggtggagctgtgcacacaagaggactggatttcgcctgtgatatctacat
ttgggccccgctcgcaggcacatgtggagtgc
CAR E6 Fc/8/4-1BB/CD3z sequence:
N-CD8ls-huMNE6scFv-Human IgG1 Fc- CD8 transmembrane- 4-1BB- CD3zeta-C
(DNA)
(SEQ ID NO: 310)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggcggaggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaagagcccaaatcttgtgac
aaaactcacacatgcccaccgtgcccagcacctgaactcctggggggaccgtcagtcttcctcttccccccaaaacccaag
gacaccctcatgatctcccggacccctgaggtcacatgcgtggtggtggacgtgagccacgaagaccctgaggtcaagttc
aactggtacgtggacggcgtggaggtgcataatgccaagacaaagccgcgggaggagcagtacaacagcacgtaccgtgtg
gtcagcgtcctcaccgtcctgcaccaggactggctgaatggcaaggagtacaagtgcaaggtctccaacaaagccctccca
gcccccatcgagaaaaccatctccaaagccaaagggcagccccgagaaccacaggtgtacaccctgcccccatcccgggag
gagatgaccaagaaccaggtcagcctgacctgcctggtcaaaggcttctatcccagcgacatcgccgtggagtgggagagc
aatgggcagccggagaacaactacaagaccacgcctcccgtgctggactccgacggctccttcttcctctacagcaagctc
accgtggacaagagcaggtggcagcaggggaacgtcttctcatgctccgtgatgcatgaggctctgcacaaccactacacg
cagaagagcctctccctgtctccgggtaaaatctacatttgggccccgctcgcaggcacatgtggagtgctcctcctctcc
ctggtgattaccctgtactgcaaaaggggccgcaaaaaactcctttacatttttaagcagccttttatgaggccagtacag
acgactcaagaggaagacgggtgctcatgccgctttcctgaggaggaggaaggagggtgcgaactgcgcgttaagttctcc
cgatcagccgacgcgcctgcttacaagcagggccagaaccaactgtacaacgagctgaatctcggtagacgggaagagtac
gacgtgttggacaaacggagaggccgcgacccagaaatgggcggcaagcctcgcaggaaaaacccccaggagggactgtac
aatgagttgcagaaagataagatggcagaagcttatagcgagatcggaatgaagggggaaaggagacgagggaaaggacac
gacggcctttatcagggcctgtccacagcaacaaaagatacgtatgacgccctccatatgcaggcacttccaccacggtga
taa
(amino acids)
(SEQ ID NO: 311)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLTVDKSRWQQGNVFSCSVMHEA
LHNHYTQKSLSLSPGKIYIWAPLAGTCGVLLLSLVITLYCKRGRKKLLYIFKQPFMRPVQTTQEEDGCSCRFPEEEEGGC
ELRVKFSRSADAPAYKQGQNQLYNELNLGRREEYDVLDKRRGRDPEMGGKPRRKNPQEGLYNELQKDKMAEAYSEIGMKG
ERRRGKGHDGLYQGLSTATKDTYDALHMQALPPR**
E6 CAR pCDH gBLOCK sequence:
(DNA)
(SEQ ID NO: 312)
acgctgttttgacctccatagaagattctagagctagctgtagagcttggtaccgagggccaccatggccctgcccgtgac
cgctttgctgctccccctggcgctgctgctgcacgccgccaggccagaggtccagctggttgagagtggcggtgggctggt
taagcctggcggctccctgcggctgagctgcgccgcgagtggatttactttcagccgatatgggatgagttgggtgcggca
agctcccgggaagaggctggaatgggtctcaacaatctccggggggggcacttacatctattaccccgactcagtcaaggg
gagatttaccatttcacgagacaacgctaagaataccctgtatttgcagatgaattctctgagagcagaggacacagctgt
ttactattgtacccgcgacaactatggcaggaactacgactacggtatggactattggggacaagggacattggttacagt
gagcagtggcggcgggggcagcggaggaggaggcagcggtggcggaggcagcgagatagtgctcacgcagtcacccgcgac
tctcagtctctcacctggggaacgagctaccctgacgtgctctgctacctcctcagtgtcatatattcactggtatcagca
acggcccgggcagtcccctagattgctcatttatagtacctctaatctggcctcaggtatccctgc
E6 CAR Fc pCDH gBLOCK sequence:
(DNA)
(SEQ ID NO: 313)
agtacctctaatctggcctcaggtatccctgcacgattttctggatctggttcaggttctgattacaccctcactatctct
agcctggagcctgaagactttgccgtttattactgccagcagaggtctagctccccattcacctttgggagtgggaccaag
gttgaaattaaagagcccaaatcttgtgacaaaactcacacatgcccaccgtgcccagcacctgaactcctggggggaccg
tcagtcttcctcttccccccaaaacccaaggacaccctcatgatctcccggacccctgaggtcacatgcgtggtggtggac
gtgagccacgaagaccctgaggtcaagttcaactggtacgtggacggcgtggaggtgcataatgccaagacaaagccgcgg
gaggagcagtacaacagcacgtaccgtgtggtcagcgtcctcaccgtcctgcaccaggactggctgaatggcaaggagtac
aagtgcaaggtctccaacaaagccctcccagcccccatcgagaaaaccatctccaaagccaaagggcagccccgagaacca
caggtgtacaccctgcccccatcccgggaggagatgaccaagaaccaggtcagcctgacctgcctggtcaaaggcttctat
cccagcgacatcgccgtggagtgggagagcaatgggcagccggagaacaactacaagaccacgcctcccgtgctg
E6 CAR 8BB3 pCDH gBLOCK sequence:
(DNA)
(SEQ ID NO: 314)
agaacaactacaagaccacgcctcccgtgctggactccgacggctccttcttcctctacagcaagctcaccgtggacaaga
gcaggtggcagcaggggaacgtcttctcatgctccgtgatgcatgaggctctgcacaaccactacacgcagaagagcctct
ccctgtctccgggtaaaatctacatttgggccccgctcgcaggcacatgtggagtgctcctcctctccctggtgattaccc
tgtactgcaaaaggggccgcaaaaaactcctttacatttttaagcagccttttatgaggccagtacagacgactcaagagg
aagacgggtgctcatgccgctttcctgaggaggaggaaggagggtgcgaactgcgcgttaagttctcccgatcagccgacg
cgcctgcttacaagcagggccagaaccaactgtacaacgagctgaatctcggtagacgggaagagtacgacgtgttggaca
aacggagaggccgcgacccagaaatgggcggcaagcctcgcaggaaaaacccccaggagggactgtacaatgagttgcaga
aagataagatggcagaagcttatagcgagatcggaatgaagggggaaaggagacgagggaaaggacacgacggcctttatc
agggcctgtccacagcaacaaaagatacgtatgacgccctccatatgcaggcacttccaccacggtgataagtttaaaccc
gctgatcaggcggccgcgaaggatctgcgatcgctccggtgcccgtcag
CAR E6 FcH/8/4-1BB/CD3z sequence:
N-CD8ls-huMNE6scFv-Human IgG1 hingeless Fc Y407R- CD8 transmembrane- 4-1BB-
CD3zeta-C
(DNA)
(SEQ ID NO: 315)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggcggaggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaagcacctgaactcctgggg
ggaccgtcagtcttcctcttccccccaaaacccaaggacaccctcatgatctcccggacccctgaggtcacatgcgtggtg
gtggacgtgagccacgaagaccctgaggtcaagttcaactggtacgtggacggcgtggaggtgcataatgccaagacaaag
ccgcgggaggagcagtacaacagcacgtaccgtgtggtcagcgtcctcaccgtcctgcaccaggactggctgaatggcaag
gagtacaagtgcaaggtctccaacaaagccctcccagcccccatcgagaaaaccatctccaaagccaaagggcagccccga
gaaccacaggtgtacaccctgcccccatcccgggaggagatgaccaagaaccaggtcagcctgacctgcctggtcaaaggc
ttctatcccagcgacatcgccgtggagtgggagagcaatgggcagccggagaacaactacaagaccacgcctcccgtgctg
gactccgacggctccttcttcctctacagcaagctcaccgtggacaagagcaggtggcagcaggggaacgtcttctcatgc
tccgtgatgcatgaggctctgcacaaccactacacgcagaagagcctctccctgtctccgggtaaaatctacatttgggcc
ccgctcgcaggcacatgtggagtgctcctcctctccctggtgattaccctgtactgcaaaaggggccgcaaaaaactcctt
tacatttttaagcagccttttatgaggccagtacagacgactcaagaggaagacgggtgctcatgccgctttcctgaggag
gaggaaggagggtgcgaactgcgcgttaagttctcccgatcagccgacgcgcctgcttacaagcagggccagaaccaactg
tacaacgagctgaatctcggtagacgggaagagtacgacgtgttggacaaacggagaggccgcgacccagaaatgggcggc
aagcctcgcaggaaaaacccccaggagggactgtacaatgagttgcagaaagataagatggcagaagcttatagcgagatc
ggaatgaagggggaaaggagacgagggaaaggacacgacggcctttatcagggcctgtccacagcaacaaaagatacgtat
gacgccctccatatgcaggcacttccaccacggtgataa
(amino acids)
(SEQ ID NO: 316)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKAPELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKSRWQQGNVFSCSVMHEALHNHYTQKSLSLSPG
KIYIWAPLAGTCGVLLLSLVITLYCKRGRKKLLYIFKQPFMRPVQTTQEEDGCSCRFPEEEEGGCELRVKFSRSADAPAY
KQGQNQLYNELNLGRREEYDVLDKRRGRDPEMGGKPRRKNPQEGLYNELQKDKMAEAYSEIGMKGERRRGKGHDGLYQGL
STATKDTYDALHMQALPPR**
E6 CAR FcH pCDH gBLOCK sequence:
(DNA)
(SEQ ID NO: 317)
agtacctctaatctggcctcaggtatccctgcacgattttctggatctggttcaggttctgattacaccctcactatctct
agcctggagcctgaagactttgccgtttattactgccagcagaggtctagctccccattcacctttgggagtgggaccaag
gttgaaattaaagcacctgaactcctggggggaccgtcagtcttcctcttccccccaaaacccaaggacaccctcatgatc
tcccggacccctgaggtcacatgcgtggtggtggacgtgagccacgaagaccctgaggtcaagttcaactggtacgtggac
ggcgtggaggtgcataatgccaagacaaagccgcgggaggagcagtacaacagcacgtaccgtgtggtcagcgtcctcacc
gtcctgcaccaggactggctgaatggcaaggagtacaagtgcaaggtctccaacaaagccctcccagcccccatcgagaaa
accatctccaaagccaaagggcagccccgagaaccacaggtgtacaccctgcccccatcccgggaggagatgaccaagaac
caggtcagcctgacctgcctggtcaaaggcttctatcccagcgacatcgccgtggagtgggagagcaatgggcagccggag
aacaactacaagaccacgcctcccgtgctg
CAR E6 Fc/4/4-1BB/CD3z sequence:
N-CD8ls-huMNE6scFv-Human IgG1 Fc- CD4 transmembrane- CD28- 4-1BB- CD3zeta-C
(DNA)
(SEQ ID NO: 318)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggcggaggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaagagcccaaatcttgtgac
aaaactcacacatgcccaccgtgcccagcacctgaactcctggggggaccgtcagtcttcctcttccccccaaaacccaag
gacaccctcatgatctcccggacccctgaggtcacatgcgtggtggtggacgtgagccacgaagaccctgaggtcaagttc
aactggtacgtggacggcgtggaggtgcataatgccaagacaaagccgcgggaggagcagtacaacagcacgtaccgtgtg
gtcagcgtcctcaccgtcctgcaccaggactggctgaatggcaaggagtacaagtgcaaggtctccaacaaagccctccca
gcccccatcgagaaaaccatctccaaagccaaagggcagccccgagaaccacaggtgtacaccctgcccccatcccgggag
gagatgaccaagaaccaggtcagcctgacctgcctggtcaaaggcttctatcccagcgacatcgccgtggagtgggagagc
aatgggcagccggagaacaactacaagaccacgcctcccgtgctggactccgacggctccttcttcctctacagcaagctc
accgtggacaagagcaggtggcagcaggggaacgtcttctcatgctccgtgatgcatgaggctctgcacaaccactacacg
cagaagagcctctccctgtctccgggtaaaatggccctgattgtgctggggggcgtcgccggcctcctgcttttcattggg
ctaggcatcttcttcaaaaggggccgcaaaaaactcctttacatttttaagcagccttttatgaggccagtacagacgact
caagaggaagacgggtgctcatgccgctttcctgaggaggaggaaggagggtgcgaactgcgcgttaagttctcccgatca
gccgacgcgcctgcttacaagcagggccagaaccaactgtacaacgagctgaatctcggtagacgggaagagtacgacgtg
ttggacaaacggagaggccgcgacccagaaatgggcggcaagcctcgcaggaaaaacccccaggagggactgtacaatgag
ttgcagaaagataagatggcagaagcttatagcgagatcggaatgaagggggaaaggagacgagggaaaggacacgacggc
ctttatcagggcctgtccacagcaacaaaagatacgtatgacgccctccatatgcaggcacttccaccacggtgataa
(amino acids)
(SEQ ID NO: 319)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLTVDKSRWQQGNVFSCSVMHEA
LHNHYTQKSLSLSPGKMALIVLGGVAGLLLFIGLGIFFKRGRKKLLYIFKQPFMRPVQTTQEEDGCSCRFPEEEEGGCEL
RVKFSRSADAPAYKQGQNQLYNELNLGRREEYDVLDKRRGRDPEMGGKPRRKNPQEGLYNELQKDKMAEAYSEIGMKGER
RRGKGHDGLYQGLSTATKDTYDALHMQALPPR**
E6 CAR 44BB3 pCDH gBLOCK sequence:
(DNA)
(SEQ ID NO: 320)
agaacaactacaagaccacgcctcccgtgctggactccgacggctccttcttcctctacagcaagctcaccgtggacaaga
gcaggtggcagcaggggaacgtcttctcatgctccgtgatgcatgaggctctgcacaaccactacacgcagaagagcctct
ccctgtctccgggtaaaatggccctgattgtgctggggggcgtcgccggcctcctgcttttcattgggctaggcatcttct
tcaaaaggggccgcaaaaaactcctttacatttttaagcagccttttatgaggccagtacagacgactcaagaggaagacg
ggtgctcatgccgctttcctgaggaggaggaaggagggtgcgaactgcgcgttaagttctcccgatcagccgacgcgcctg
cttacaagcagggccagaaccaactgtacaacgagctgaatctcggtagacgggaagagtacgacgtgttggacaaacgga
gaggccgcgacccagaaatgggcggcaagcctcgcaggaaaaacccccaggagggactgtacaatgagttgcagaaagata
agatggcagaagcttatagcgagatcggaatgaagggggaaaggagacgagggaaaggacacgacggcctttatcagggcc
tgtccacagcaacaaaagatacgtatgacgccctccatatgcaggcacttccaccacggtgataagtttaaacccgctgat
caggcggccgcgaaggatctgcgatcgctccggtgcccgtcag
CAR E6 FcH/4/4-1BB/CD3z sequence:
N-CD8ls-huMNE6scFv-Human IgG1 hingeless Fc Y407R- CD4 transmembrane- CD28-
4-1BB-CD3zeta-C
(DNA)
(SEQ ID NO: 321)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggcggaggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaagcacctgaactcctgggg
ggaccgtcagtcttcctcttccccccaaaacccaaggacaccctcatgatctcccggacccctgaggtcacatgcgtggtg
gtggacgtgagccacgaagaccctgaggtcaagttcaactggtacgtggacggcgtggaggtgcataatgccaagacaaag
ccgcgggaggagcagtacaacagcacgtaccgtgtggtcagcgtcctcaccgtcctgcaccaggactggctgaatggcaag
gagtacaagtgcaaggtctccaacaaagccctcccagcccccatcgagaaaaccatctccaaagccaaagggcagccccga
gaaccacaggtgtacaccctgcccccatcccgggaggagatgaccaagaaccaggtcagcctgacctgcctggtcaaaggc
ttctatcccagcgacatcgccgtggagtgggagagcaatgggcagccggagaacaactacaagaccacgcctcccgtgctg
gactccgacggctccttcttcctctacagcaagctcaccgtggacaagagcaggtggcagcaggggaacgtcttctcatgc
tccgtgatgcatgaggctctgcacaaccactacacgcagaagagcctctccctgtctccgggtaaaatggccctgattgtg
ctggggggcgtcgccggcctcctgcttttcattgggctaggcatcttcttcaaaaggggccgcaaaaaactcctttacatt
tttaagcagccttttatgaggccagtacagacgactcaagaggaagacgggtgctcatgccgctttcctgaggaggaggaa
ggagggtgcgaactgcgcgttaagttctcccgatcagccgacgcgcctgcttacaagcagggccagaaccaactgtacaac
gagctgaatctcggtagacgggaagagtacgacgtgttggacaaacggagaggccgcgacccagaaatgggcggcaagcct
cgcaggaaaaacccccaggagggactgtacaatgagttgcagaaagataagatggcagaagcttatagcgagatcggaatg
aagggggaaaggagacgagggaaaggacacgacggcctttatcagggcctgtccacagcaacaaaagatacgtatgacgcc
ctccatatgcaggcacttccaccacggtgataa
(amino acids)
(SEQ ID NO: 322)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKAPELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKSRWQQGNVFSCSVMHEALHNHYTQKSLSLSPG
KMALIVLGGVAGLLLFIGLGIFFKRGRKKLLYIFKQPFMRPVQTTQEEDGCSCRFPEEEEGGCELRVKFSRSADAPAYKQ
GQNQLYNELNLGRREEYDVLDKRRGRDPEMGGKPRRKNPQEGLYNELQKDKMAEAYSEIGMKGERRRGKGHDGLYQGLST
ATKDTYDALHMQALPPR**
CAR E6 IgD/8/4-1BB/CD3z sequence:
N-CD8ls-huMNE6scFv-IgD hinge region- CD8 transmembrane- 4-1BB- CD3zeta-C
(DNA)
(SEQ ID NO: 323)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggcggaggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaagagtctccaaaggcaca
ggcctcctcagtgcccactgcacaaccccaagcagagggcagcctcgccaaggcaaccacagccccagccaccacccgta
acacaggaagaggcggcgaagagaagaaaaaggagaaggagaaagaggaacaagaagagagagagacaaagacaccaatc
tacatttgggccccgctcgcaggcacatgtggagtgctcctcctctccctggtgattaccctgtactgcaaaaggggccgc
aaaaaactcctttacatttttaagcagccttttatgaggccagtacagacgactcaagaggaagacgggtgctcatgccgc
tttcctgaggaggaggaaggagggtgcgaactgcgcgttaagttctcccgatcagccgacgcgcctgcttacaagcagggc
cagaaccaactgtacaacgagctgaatctcggtagacgggaagagtacgacgtgttggacaaacggagaggccgcgaccca
gaaatgggcggcaagcctcgcaggaaaaacccccaggagggactgtacaatgagttgcagaaagataagatggcagaagct
tatagcgagatcggaatgaagggggaaaggagacgagggaaaggacacgacggcctttatcagggcctgtccacagcaaca
aaagatacgtatgacgccctccatatgcaggcacttccaccacggtgataa
(amino acids)
(SEQ ID NO: 324)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKESPKAQASSVPTAQPQAEGSLAKATTAPATTRNTGRGGEEKKKEKEKEEQEERETK
TPIYIWAPLAGTCGVLLLSLVITLYCKRGRKKLLYIFKQPFMRPVQTTQEEDGCSCRFPEEEEGGCELRVKFSRSADAPA
YKQGQNQLYNELNLGRREEYDVLDKRRGRDPEMGGKPRRKNPQEGLYNELQKDKMAEAYSEIGMKGERRRGKGHDGLYQG
LSTATKDTYDALHMQALPPR**
E6 CAR IgD8 pcDH gBLOCK sequence:
(DNA)
(SEQ ID NO: 325)
agtacctctaatctggcctcaggtatccctgcacgattttctggatctggttcaggttctgattacaccctcactatctct
agcctggagcctgaagactttgccgtttattactgccagcagaggtctagctccccattcacctttgggagtgggaccaag
gttgaaattaaagagtctccaaaggcacaggcctcctcagtgcccactgcacaaccccaagcagagggcagcctcgccaa
ggcaaccacagccccagccaccacccgtaacacaggaagaggcggcgaagagaagaaaaaggagaaggagaaagaggaac
aagaagagagagagacaaagacaccaatctacatttgggccccgctcgcaggcacatgtggagtgctcctcctctccctg
gtgattaccctgtactgcaaaaggggccgcaaaaaactcctttacatttttaagcagccttttatgaggccag
E6 CAR BB 3 pCDH gBLOCK sequence:
(DNA)
(SEQ ID NO: 326)
acatttttaagcagccttttatgaggccagtacagacgactcaagaggaagacgggtgctcatgccgctttcctgaggagg
aggaaggagggtgcgaactgcgcgttaagttctcccgatcagccgacgcgcctgcttacaagcagggccagaaccaactgt
acaacgagctgaatctcggtagacgggaagagtacgacgtgttggacaaacggagaggccgcgacccagaaatgggcggca
agcctcgcaggaaaaacccccaggagggactgtacaatgagttgcagaaagataagatggcagaagcttatagcgagatcg
gaatgaagggggaaaggagacgagggaaaggacacgacggcctttatcagggcctgtccacagcaacaaaagatacgtatg
acgccctccatatgcaggcacttccaccacggtgataagtttaaacccgctgatcaggcggccgcgaaggatctgcgatcg
ctccggtgcccgtcag
CAR E6 IgD/4/4-1BB/CD3z sequence:
N-CD8ls-huMNE6scFv-IgD hinge region- CD4 transmembrane- 4-1BB- CD3zeta-C
(DNA)
(SEQ ID NO: 327)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggcggaggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaagagtctccaaaggcaca
ggcctcctcagtgcccactgcacaaccccaagcagagggcagcctcgccaaggcaaccacagccccagccaccacccgta
acacaggaagaggcggcgaagagaagaaaaaggagaaggagaaagaggaacaagaagagagagagacaaagacaccaatg
gccctgattgtgctggggggcgtcgccggcctcctgcttttcattgggctaggcatcttcttcaaaaggggccgcaaaaaa
ctcctttacatttttaagcagccttttatgaggccagtacagacgactcaagaggaagacgggtgctcatgccgctttcct
gaggaggaggaaggagggtgcgaactgcgcgttaagttctcccgatcagccgacgcgcctgcttacaagcagggccagaac
caactgtacaacgagctgaatctcggtagacgggaagagtacgacgtgttggacaaacggagaggccgcgacccagaaatg
ggcggcaagcctcgcaggaaaaacccccaggagggactgtacaatgagttgcagaaagataagatggcagaagcttatagc
gagatcggaatgaagggggaaaggagacgagggaaaggacacgacggcctttatcagggcctgtccacagcaacaaaagat
acgtatgacgccctccatatgcaggcacttccaccacggtgataa
(amino acids)
(SEQ ID NO: 328)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKESPKAQASSVPTAQPQAEGSLAKATTAPATTRNTGRGGEEKKKEKEKEEQEERETK
TPMALIVLGGVAGLLLFIGLGIFFKRGRKKLLYIFKQPFMRPVQTTQEEDGCSCRFPEEEEGGCELRVKFSRSADAPAYK
QGQNQLYNELNLGRREEYDVLDKRRGRDPEMGGKPRRKNPQEGLYNELQKDKMAEAYSEIGMKGERRRGKGHDGLYQGLS
TATKDTYDALHMQALPPR**
E6 CAR IgD4 pcDH gBLOCK sequence:
(DNA)
(SEQ ID NO: 329)
agtacctctaatctggcctcaggtatccctgcacgattttctggatctggttcaggttctgattacaccctcactatctct
agcctggagcctgaagactttgccgtttattactgccagcagaggtctagctccccattcacctttgggagtgggaccaag
gttgaaattaaagagtctccaaaggcacaggcctcctcagtgcccactgcacaaccccaagcagagggcagcctcgccaa
ggcaaccacagccccagccaccacccgtaacacaggaagaggcggcgaagagaagaaaaaggagaaggagaaagaggaac
aagaagagagagagacaaagacaccaatggccctgattgtgctggggggcgtcgccggcctcctgcttttcattgggcta
ggcatcttcttcaaaaggggccgcaaaaaactcctttacatttttaagcagccttttatgaggccag
CAR E6 X4/8/4-1BB/CD3z sequence:
N-CD8ls-huMNE6scFv- X4 linker- CD8 transmembrane- 4-1BB- CD3zeta-C
(DNA)
(SEQ ID NO: 330)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggcggaggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaagacaagacgcacaccaag
ccacctaaaccagctccagaactgctcggaggtcctggcaccggaaccggaggacctaccatcaaaccacctaagccacct
aagcctgctcctaacctgctcggaggacctatctacatttgggccccgctcgcaggcacatgtggagtgctcctcctctcc
ctggtgattaccctgtactgcaaaaggggccgcaaaaaactcctttacatttttaagcagccttttatgaggccagtacag
acgactcaagaggaagacgggtgctcatgccgctttcctgaggaggaggaaggagggtgcgaactgcgcgttaagttctcc
cgatcagccgacgcgcctgcttacaagcagggccagaaccaactgtacaacgagctgaatctcggtagacgggaagagtac
gacgtgttggacaaacggagaggccgcgacccagaaatgggcggcaagcctcgcaggaaaaacccccaggagggactgtac
aatgagttgcagaaagataagatggcagaagcttatagcgagatcggaatgaagggggaaaggagacgagggaaaggacac
gacggcctttatcagggcctgtccacagcaacaaaagatacgtatgacgccctccatatgcaggcacttccaccacggtga
taa
(amino acids)
(SEQ ID NO: 331)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKDKTHTKPPKPAPELLGGPGTGTGGPTIKPPKPPKPAPNLLGGPIYIWAPLAGTCGV
LLLSLVITLYCKRGRKKLLYIFKQPFMRPVQTTQEEDGCSCRFPEEEEGGCELRVKFSRSADAPAYKQGQNQLYNELNLG
RREEYDVLDKRRGRDPEMGGKPRRKNPQEGLYNELQKDKMAEAYSEIGMKGERRRGKGHDGLYQGLSTATKDTYDALHMQ
ALPPR**
E6 CAR X48 pCDH gBLOCK sequence:
(DNA)
(SEQ ID NO: 332)
agtacctctaatctggcctcaggtatccctgcacgattttctggatctggttcaggttctgattacaccctcactatctct
agcctggagcctgaagactttgccgtttattactgccagcagaggtctagctccccattcacctttgggagtgggaccaag
gttgaaattaaagacaagacgcacaccaagccacctaaaccagctccagaactgctcggaggtcctggcaccggaaccgga
ggacctaccatcaaaccacctaagccacctaagcctgctcctaacctgctcggaggacctatctacatttgggccccgctc
gcaggcacatgtggagtgctcctcctctccctggtgattaccctgtactgcaaaaggggccgcaaaaaactcctttacatt
tttaagcagccttttatgaggccag
CAR E6 X4/4/4-1BB/CD3z sequence:
N-CD8ls-huMNE6scFv-X4 linker- CD4 transmembrane- 4-1BB- CD3zeta-C
(DNA)
(SEQ ID NO: 333)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggcggaggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaagacaagacgcacaccaag
ccacctaaaccagctccagaactgctcggaggtcctggcaccggaaccggaggacctaccatcaaaccacctaagccacct
aagcctgctcctaacctgctcggaggacctatggccctgattgtgctggggggcgtcgccggcctcctgcttttcattggg
ctaggcatcttcttcaaaaggggccgcaaaaaactcctttacatttttaagcagccttttatgaggccagtacagacgact
caagaggaagacgggtgctcatgccgctttcctgaggaggaggaaggagggtgcgaactgcgcgttaagttctcccgatca
gccgacgcgcctgcttacaagcagggccagaaccaactgtacaacgagctgaatctcggtagacgggaagagtacgacgtg
ttggacaaacggagaggccgcgacccagaaatgggcggcaagcctcgcaggaaaaacccccaggagggactgtacaatgag
ttgcagaaagataagatggcagaagcttatagcgagatcggaatgaagggggaaaggagacgagggaaaggacacgacggc
ctttatcagggcctgtccacagcaacaaaagatacgtatgacgccctccatatgcaggcacttccaccacggtgataa
(amino acids)
(SEQ ID NO: 334)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKDKTHTKPPKPAPELLGGPGTGTGGPTIKPPKPPKPAPNLLGGPMALIVLGGVAGLL
LFIGLGIFFKRGRKKLLYIFKQPFMRPVQTTQEEDGCSCRFPEEEEGGCELRVKFSRSADAPAYKQGQNQLYNELNLGRR
EEYDVLDKRRGRDPEMGGKPRRKNPQEGLYNELQKDKMAEAYSEIGMKGERRRGKGHDGLYQGLSTATKDTYDALHMQAL
PPR**
E6 CAR X44 pCDH gBLOCK sequence:
(DNA)
(SEQ ID NO: 335)
agtacctctaatctggcctcaggtatccctgcacgattttctggatctggttcaggttctgattacaccctcactatctct
agcctggagcctgaagactttgccgtttattactgccagcagaggtctagctccccattcacctttgggagtgggaccaag
gttgaaattaaagacaagacgcacaccaagccacctaaaccagctccagaactgctcggaggtcctggcaccggaaccgga
ggacctaccatcaaaccacctaagccacctaagcctgctcctaacctgctcggaggacctatggccctgattgtgctgggg
ggcgtcgccggcctcctgcttttcattgggctaggcatcttcttcaaaaggggccgcaaaaaactcctttacatttttaag
cagccttttatgaggccag
CAR E6 8 + 4/4/4-1BB/CD3z sequence:
N-CD8ls-huMNE6scFv- CD8ecd + CD4ecd fragment - CD4 transmembrane- 4-1BB-
CD3zeta-C
(DNA)
(SEQ ID NO: 336)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggcggaggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaaacgacaaccccggccccc
agaccaccaacgccagcccccaccatcgccagccaacccctgtctctgagaccagaagcctgtaggcctgccgccggtgga
gctgtgcacacaagaggactggatttcgcctgtgatatggccctgattgtgctggggggcgtcgccggcctcctgcttttc
attgggctaggcatcttcttcaaaaggggccgcaaaaaactcctttacatttttaagcagccttttatgaggccagtacag
acgactcaagaggaagacgggtgctcatgccgctttcctgaggaggaggaaggagggtgcgaactgcgcgttaagttctcc
cgatcagccgacgcgcctgcttacaagcagggccagaaccaactgtacaacgagctgaatctcggtagacgggaagagtac
gacgtgttggacaaacggagaggccgcgacccagaaatgggcggcaagcctcgcaggaaaaacccccaggagggactgtac
aatgagttgcagaaagataagatggcagaagcttatagcgagatcggaatgaagggggaaaggagacgagggaaaggacac
gacggcctttatcagggcctgtccacagcaacaaaagatacgtatgacgccctccatatgcaggcacttccaccacggtga
taa
(amino acids)
(SEQ ID NO: 337)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKTTTPAPRPPTPAPTIASQPLSLRPEACRPAAGGAVHTRGLDFACDMALIVLGGVAG
LLLFIGLGIFFKRGRKKLLYIFKQPFMRPVQTTQEEDGCSCRFPEEEEGGCELRVKFSRSADAPAYKQGQNQLYNELNLG
RREEYDVLDKRRGRDPEMGGKPRRKNPQEGLYNELQKDKMAEAYSEIGMKGERRRGKGHDGLYQGLSTATKDTYDALHMQ
ALPPR**
E6 CAR CD844 pCDH gBLOCK sequence:
(DNA)
(SEQ ID NO: 338)
agtacctctaatctggcctcaggtatccctgcacgattttctggatctggttcaggttctgattacaccctcactatctct
agcctggagcctgaagactttgccgtttattactgccagcagaggtctagctccccattcacctttgggagtgggaccaag
gttgaaattaaaacgacaaccccggcccccagaccaccaacgccagcccccaccatcgccagccaacccctgtctctgaga
ccagaagcctgtaggcctgccgccggtggagctgtgcacacaagaggactggatttcgcctgtgatatggccctgattgtg
ctggggggcgtcgccggcctcctgcttttcattgggctaggcatcttcttcaaaaggggccgcaaaaaactcctttacatt
tttaagcagccttttatgaggccag
Humanized C2 scFV sequence in CAR:
(DNA)
(SEQ ID NO: 339)
gagggccaccatggccttgccagtgacggccctgctgctgccattggctcttctgttgcacgctgccaggcctgaagtgca
gctcgtagagagtggcgggggactggtgaagcccggtggaagcctcagactcagttgcgccgcctcaggtttcactttttc
aggttacgccatgtcctgggtaagacaggcaccggggaaaggactcgagtgggtgtctactatcagctcaggaggcactta
tatatattatcctgactctgtaaaaggccgatttacgatttctcgcgacaatgcaaagaactccctctacctccaaatgaa
cagtcttagggcagaagacactgctgtatactattgtgcacgcctcggcggcgacaactactacgagtactttgacgtgtg
ggggaaagggactaccgtgacagtttcaagcggaggaggtggctcaggtggaggcgggtcaggggggggaggaagtgatat
tgtgctcacacaatccccagcctccctggctgtgtctcccggccaacgcgctacaattacatgtcgggcctccaaaagcgt
gagcaccagcggctacagctacatgcactggtatcaacagaaaccaggacaaccccccaaactgttgatttatctcgcttc
aaacttggagtccggcgtgcctgcgcgcttttcagggagtgggagcggcacagattttacgctgactatcaaccccgtaga
agcaaacgatacagcgaattattattgtcaacattcccgggaactcccctttacgttcggcgggggcacaaaggtcgaaat
taagagaacc
(amino acids)
(SEQ ID NO: 340)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRT
Humanized E6 scFV sequence in CAR:
(DNA)
(SEQ ID NO: 341)
gaggtccagctggttgagagtggcggtgggctggttaagcctggcggctccctgcggctgagctgcgccgcgagtggattt
actttcagccgatatgggatgagttgggtgcggcaagctcccgggaagaggctggaatgggtctcaacaatctccgggggg
ggcacttacatctattaccccgactcagtcaaggggagatttaccatttcacgagacaacgctaagaataccctgtatttg
cagatgaattctctgagagcagaggacacagctgtttactattgtacccgcgacaactatggcaggaactacgactacggt
atggactattggggacaagggacattggttacagtgagcagtggcggcgggggcagcggaggaggaggcagcggtgggggg
ggcagcgagatagtgctcacgcagtcacccgcgactctcagtctctcacctggggaacgagctaccctgacgtgctctgct
acctcctcagtgtcatatattcactggtatcagcaacggcccgggcagtcccctagattgctcatttatagtacctctaat
ctggcctcaggtatccctgcacgattttctggatctggttcaggttctgattacaccctcactatctctagcctggagcct
gaagactttgccgtttattactgccagcagaggtctagctccccattcacctttgggagtgggaccaaggttgaaattaaa
(amino acids)
(SEQ ID NO: 342)
EVQLVESGGGLVKPGGSLRLSCAASGFTFSRYGMSWVRQAPGKRLEWVSTISGGGTYIYYPDSVKGRFTISRDNAKNTLY
LQMNSLRAEDTAVYYCTRDNYGRNYDYGMDYWGQGTLVTVSSGGGGSGGGGSGGGGSEIVLTQSPATLSLSPGERATLTC
SATSSVSYIHWYQQRPGQSPRLLIYSTSNLASGIPARFSGSGSGSDYTLTISSLEPEDFAVYYCQQRSSSPFTFGSGTKV
EIK
CD8 leader sequence:
(DNA)
(SEQ ID NO: 343)
atggccctgcccgtgaccgctttgctgctccccctggcgctgctgctgcacgccgccaggcca
(amino acids)
(SEQ ID NO: 344)
MALPVTALLLPLALLLHAARP
CD8 hinge domain sequence:
(DNA)
(SEQ ID NO: 345)
acgacaaccccggcccccagaccaccaacgccagcccccaccatcgccagccaacccctgtctctgagaccagaagcctgt
aggcctgccgccggtggagctgtgcacacaagaggactggatttcgcctgtgat
(amino acids)
(SEQ ID NO: 346)
TTTPAPRPPTPAPTIASQPLSLRPEACRPAAGGAVHTRGLDFACD
CD4 hinge domain sequence:
(DNA)
(SEQ ID NO: 347)
tcgggacaggtcctgctggaatccaacatcaaggttctgcccacatggtccaccccggtgcagcca
(amino acids)
(SEQ ID NO: 348)
SGQVLLESNIKVLPTWSTPVQP
CD28 hinge domain sequence:
(DNA)
(SEQ ID NO: 349)
aaacacctttgtccaagtcccctatttcccggaccttctaagccc
(amino acids)
(SEQ ID NO: 350)
KHLCPSPLFPGPSKP
CD8 + CD4 hinge domain sequence:
(DNA)
(SEQ ID NO: 351)
acgacaaccccggcccccagaccaccaacgccagcccccaccatcgccagccaacccctgtctctgagaccagaagcctgt
aggcctgccgccggtggagctgtgcacacaagaggactggatttcgcctgtgattcgggacaggtcctgctggaatccaac
atcaaggttctgcccacatggtccaccccggtgcagcca
(amino acids)
(SEQ ID NO: 352)
TTTPAPRPPTPAPTIASQPLSLRPEACRPAAGGAVHTRGLDFACDSGQVLLESNIKVLPTWSTPVQP
CD8 + CD28 hinge domain sequence:
(DNA)
(SEQ ID NO: 353)
acgacaaccccggcccccagaccaccaacgccagcccccaccatcgccagccaacccctgtctctgagaccagaagcctgt
aggcctgccgccggtggagctgtgcacacaagaggactggatttcgcctgtgataaacacctttgtccaagtcccctatt
tcccggaccttctaagccc
(amino acids)
(SEQ ID NO: 354)
TTTPAPRPPTPAPTIASQPLSLRPEACRPAAGGAVHTRGLDFACDKHLCPSPLFPGPSKP
CD28 + CD4 hinge domain sequence:
(DNA)
(SEQ ID NO: 355)
aaacacctttgtccaagtcccctatttcccggaccttctaagccctcgggacaggtcctgctggaatccaacatcaaggt
tctgcccacatggtccaccccggtgcagcca
(amino acids)
(SEQ ID NO: 356)
KHLCPSPLFPGPSKPSGQVLLESNIKVLPTWSTPVQP
Human IgD hinge domain sequence:
(DNA)
(SEQ ID NO: 357)
gagtctccaaaggcacaggcctcctcagtgcccactgcacaaccccaagcagagggcagcctcgccaaggcaaccacagc
cccagccaccacccgtaacacaggaagaggcggcgaagagaagaaaaaggagaaggagaaagaggaacaagaagagagag
agacaaagacacca
(amino acids)
(SEQ ID NO: 358)
ESPKAQASSVPTAQPQAEGSLAKATTAPATTRNTGRGGEEKKKEKEKEEQEERETKTP
X4 linker (IgG1 and IgG2 modified hinge region) sequence:
(DNA)
(SEQ ID NO: 359)
gacaagacgcacaccaagccacctaaaccagctccagaactgctcggaggtcctggcaccggaaccggaggacctaccatc
aaaccacctaagccacctaagcctgctcctaacctgctcggaggacct
(amino acids)
(SEQ ID NO: 360)
DKTHTKPPKPAPELLGGPGTGTGGPTIKPPKPPKPAPNLLGGP
CD3 zeta transmembrane domain sequence:
(DNA)
(SEQ ID NO: 361)
ctctgctacctgctggatggaatcctcttcatctatggtgtcattctcactgccttgttcctg
(amino acids)
(SEQ ID NO: 362)
LCYLLDGILFIYGVILTALFL
CD8 transmembrane domain sequence:
(DNA)
(SEQ ID NO: 363)
atctacatttgggccccgctcgcaggcacatgtggagtgctcctcctctccctggtgattaccctgtactgc
(amino acids)
(SEQ ID NO: 364)
IYIWAPLAGTCGVLLLSLVITLYC
CD4 transmembrane domain sequence:
(DNA)
(SEQ ID NO: 365)
atggccctgattgtgctggggggcgtcgccggcctcctgcttttcattgggctaggcatcttcttc
(amino acids)
(SEQ ID NO: 366)
MALIVLGGVAGLLLFIGLGIFF
CD28 transmembrane domain sequence:
(DNA)
(SEQ ID NO: 367)
ttttgggtgctggtggtggttggtggagtcctggcttgctatagcttgctagtaacagtggcctttattattttctgggt
g
(amino acids)
(SEQ ID NO: 368)
FWVLVVVGGVLACYSLLVTVAFIIFWV
4-1BB transmembrane domain sequence:
(DNA)
(SEQ ID NO: 369)
atcatctccttctttcttgcgctgacgtcgactgcgttgctcttcctgctgttcttcctcacgctccgtttctctgttgt
t
(amino acids)
(SEQ ID NO: 370)
IISFFLALTSTALLFLLFFLTLRFSVV
OX40 transmembrane domain sequence:
(DNA)
(SEQ ID NO: 371)
gttgccgccatcctgggcctgggcctggtgctggggctgctgggccccctggccatcctgctggccctgtacctgctc
(amino acids)
(SEQ ID NO: 372)
VAAILGLGLVLGLLGPLAILLALYLL
CD3 zeta domain sequence:
(DNA)
(SEQ ID NO: 373)
cgcgttaagttctcccgatcagccgacgcgcctgcttacaagcagggccagaaccaactgtacaacgagctgaatctcggt
agacgggaagagtacgacgtgttggacaaacggagaggccgcgacccagaaatgggcggcaagcctcgcaggaaaaacccc
caggagggactgtacaatgagttgcagaaagataagatggcagaagcttatagcgagatcggaatgaagggggaaaggaga
cgagggaaaggacacgacggcctttatcagggcctgtccacagcaacaaaagatacgtatgacgccctccatatgcaggca
cttccaccacgg
(amino acids)
(SEQ ID NO: 374)
RVKFSRSADAPAYKQGQNQLYNELNLGRREEYDVLDKRRGRDPEMGGKPRRKNPQEGLYNELQKDKMAEAYSEIGMKGERR
RGKGHDGLYQGLSTATKDTYDALHMQALPPR
CD3 zeta domain variant sequence:
(DNA)
(SEQ ID NO: 375)
agagtgaagttcagcaggagcgcagacgcccccgcgtaccagcagggccagaaccagctctataacgagctcaatctagg
acgaagagaggagtacgatgttttggacaagagacgtggccgggaccctgagatggggggaaagccgagaaggaagaacc
ctcaggaaggcctgtacaatgaactgcagaaagataagatggcggaggcctacagtgagattgggatgaaaggcgagcgc
cggaggggcaaggggcacgatggcctttaccagggtctcagtacagccaccaaggacacctacgacgcccttcacatgca
ggccctgccccctcgc
(amino acids)
(SEQ ID NO: 376)
RVKFSRSADAPAYQQGQNQLYNELNLGRREEYDVLDKRRGRDPEMGGKPRRKNPQEGLYNELQKDKMAEAYSEIGMKGER
RRGKGHDGLYQGLSTATKDTYDALHMQALPPR
CD28 domain sequence:
(DNA)
(SEQ ID NO: 377)
agaagcaagcggtctcggctcctgcattctgattacatgaacatgaccccaagaagaccaggccccaccaggaaacattac
cagccctacgctccgccacgcgacttcgctgcctaccggtcc
(amino acids)
(SEQ ID NO: 378)
RSKRSRLLHSDYMNMTPRRPGPTRKHYQPYAPPRDFAAYRS
4-1BB domain sequence:
(DNA)
(SEQ ID NO: 379)
aaaaggggccgcaaaaaactcctttacatttttaagcagccttttatgaggccagtacagacgactcaagaggaagacggg
tgctcatgccgctttcctgaggaggaggaaggagggtgcgaactg
(amino acids)
(SEQ ID NO: 380)
KRGRKKLLYIFKQPFMRPVQTTQEEDGCSCRFPEEEEGGCEL
OX40 domain sequence:
(DNA)
(SEQ ID NO: 381)
cggagggaccagaggctgccccccgatgcccacaagccccctgggggaggcagtttccggacccccatccaagaggagcag
gccgacgcccactccaccctggccaagatc
(amino acids)
(SEQ ID NO: 382)
RRDQRLPPDAHKPPGGGSFRTPIQEEQADAHSTLAKI
Humanized anti CD3 scFV clone 12F6 (VH-VL) sequence:
(DNA)
(SEQ ID NO: 383)
caggtgcagctggtgcagagcggaggtggagtggtccaacctggaagatctctgagactgagctgtaaggctagcgggta
cacgttcacatcttacacgatgcactgggtgaggcaagcccccggtaagggcctggaatggatcggatatataaacccca
gctcagggtataccaaatataatcagaagttcaaagatcggttcacgatttctgctgataaaagtaagtccaccgctttc
ctgcagatggactcactcaggccagaagatactggtgtttatttctgtgcaaggtggcaggactacgacgtgtactttga
ctattgggggcaggggacgcctgtaacagtatcaagcggcggtggcggatccggcggtggcggatccggcggtggcggat
ccgatattcagatgacccagagcccgagcagcctgagcgcgagcgtgggcgatcgcgtgaccatgacctgccgcgcgagc
agcagcgtgagctatatgcattggtatcagcagaccccgggcaaagcgccgaaaccgtggatttatgcgaccagcaacct
ggcgagcggcgtgccgagccgctttagcggcagcggcagcggcaccgattataccctgaccattagcagcctgcagccgg
aagatattgcgacctattattgccagcagtggagcagcaacccgccgacctttggccagggcaccaaactgcagattacc
cgc
(amino acids)
(SEQ ID NO: 384)
QVQLVQSGGGVVQPGRSLRLSCKASGYTFTSYTMHWVRQAPGKGLEWIGYINPSSGYTKYNQKFKDRFTISADKSKSTAF
LQMDSLRPEDTGVYFCARWQDYDVYFDYWGQGTPVTVSSGGGGSGGGGSGGGGSDIQMTQSPSSLSASVGDRVTMTCRAS
SSVSYMHWYQQTPGKAPKPWIYATSNLASGVPSRFSGSGSGTDYTLTISSLQPEDIATYYCQQWSSNPPTFGQGTKLQIT
R
Humanized anti CD3 scFV clone 12F6 (VL-VH) sequence:
(DNA)
(SEQ ID NO: 385)
gatattcagatgacccagagcccgagcagcctgagcgcgagcgtgggcgatcgcgtgaccatgacctgccgcgcgagcag
cagcgtgagctatatgcattggtatcagcagaccccgggcaaagcgccgaaaccgtggatttatgcgaccagcaacctgg
cgagcggcgtgccgagccgctttagcggcagcggcagcggcaccgattataccctgaccattagcagcctgcagccggaa
gatattgcgacctattattgccagcagtggagcagcaacccgccgacctttggccagggcaccaaactgcagattacccg
cggcggtggcggatccggcggtggcggatccggcggtggcggatcccaggtgcagctggtgcagagcggaggtggagtgg
tccaacctggaagatctctgagactgagctgtaaggctagcgggtacacgttcacatcttacacgatgcactgggtgagg
caagcccccggtaagggcctggaatggatcggatatataaaccccagctcagggtataccaaatataatcagaagttcaa
agatcggttcacgatttctgctgataaaagtaagtccaccgctttcctgcagatggactcactcaggccagaagatactg
gtgtttatttctgtgcaaggtggcaggactacgacgtgtactttgactattgggggcaggggacgcctgtaacagtatca
agc
(amino acids)
(SEQ ID NO: 386)
DIQMTQSPSSLSASVGDRVTMTCRASSSVSYMHWYQQTPGKAPKPWIYATSNLASGVPSRFSGSGSGTDYTLTISSLQPE
DIATYYCQQWSSNPPTFGQGTKLQITRGGGGSGGGGSGGGGSQVQLVQSGGGVVQPGRSLRLSCKASGYTFTSYTMHWVR
QAPGKGLEWIGYINPSSGYTKYNQKFKDRFTISADKSKSTAFLQMDSLRPEDTGVYFCARWQDYDVYFDYWGQGTPVTVS
S
Humanized anti CD3 scFV clone OKT3 (VH-VL) sequence:
(DNA)
(SEQ ID NO: 387)
caggtgcagctggtgcagagcggaggcggagtggtgcagcctggaagaagcctgcgcctgagctgcaaagcgagcggcta
tacctttacccgctataccatgcattgggtgcgccaggcgccgggcaaaggcctggaatggattggctatattaacccga
gccgcggctataccaactataaccagaaagtgaaagatcgctttaccattagcaccgataaaagcaaaagcaccgcgttt
ctgcagatggatagcctgcgcccggaagataccgcggtgtattattgcgcgcgctattatgatgatcattattgcctgga
ttattggggccagggcaccaccctgaccgtgagcagcggcggtggcggatccggcggtggcggatccggcggtggcggat
ccgatattcagatgacccagagcccgagcagcctgagcgcgagcgtgggcgatcgcgtgaccattacctgcagcgcgagc
agcagcgtgagctatatgaactggtatcagcagaccccgggcaaagcgccgaaacgctggatttatgataccagcaaact
ggcgagcggcgtgccgagccgctttagcggcagcggcagcggcaccgattatacctttaccattagcagcctgcagccgg
aagatattgcgacctattattgccagcagtggagcagcaacccgtttacctttggccagggcaccaaactgcagattacc
cgc
(amino acids)
(SEQ ID NO: 388)
QVQLVQSGGGVVQPGRSLRLSCKASGYTFTRYTMHWVRQAPGKGLEWIGYINPSRGYTNYNQKVKDRFTISTDKSKSTAF
LQMDSLRPEDTAVYYCARYYDDHYCLDYWGQGTTLTVSSGGGGSGGGGSGGGGSDIQMTQSPSSLSASVGDRVTITCSAS
SSVSYMNWYQQTPGKAPKRWIYDTSKLASGVPSRFSGSGSGTDYTFTISSLQPEDIATYYCQQWSSNPFTFGQGTKLQIT
R
Humanized anti CD3 scFV clone OKT3 (VH-VL) sequence:
(DNA)
(SEQ ID NO: 389)
gatattcagatgacccagagcccgagcagcctgagcgcgagcgtgggcgatcgcgtgaccattacctgcagcgcgagcag
cagcgtgagctatatgaactggtatcagcagaccccgggcaaagcgccgaaacgctggatttatgataccagcaaactgg
cgagcggcgtgccgagccgctttagcggcagcggcagcggcaccgattatacctttaccattagcagcctgcagccggaa
gatattgcgacctattattgccagcagtggagcagcaacccgtttacctttggccagggcaccaaactgcagattacccg
cggcggtggcggatccggcggtggcggatccggcggtggcggatcccaggtgcagctggtgcagagcggaggcggagtgg
tgcagcctggaagaagcctgcgcctgagctgcaaagcgagcggctatacctttacccgctataccatgcattgggtgcgc
caggcgccgggcaaaggcctggaatggattggctatattaacccgagccgcggctataccaactataaccagaaagtgaa
agatcgctttaccattagcaccgataaaagcaaaagcaccgcgtttctgcagatggatagcctgcgcccggaagataccg
cggtgtattattgcgcgcgctattatgatgatcattattgcctggattattggggccagggcaccaccctgaccgtgagc
agc
(amino acids)
(SEQ ID NO: 390)
DIQMTQSPSSLSASVGDRVTITCSASSSVSYMNWYQQTPGKAPKRWIYDTSKLASGVPSRFSGSGSGTDYTFTISSLQPE
DIATYYCQQWSSNPFTFGQGTKLQITRGGGGSGGGGSGGGGSQVQLVQSGGGVVQPGRSLRLSCKASGYTFTRYTMHWVR
QAPGKGLEWIGYINPSRGYTNYNQKVKDRFTISTDKSKSTAFLQMDSLRPEDTAVYYCARYYDDHYCLDYWGQGTTLTVS
S
HumanizeE6 scFV (VH-VL) sequence:
(DNA)
(SEQ ID NO: 391)
gaggtgcagctggtggagtctgggggaggcctggtcaagcctggggggtccctgagactctcctgtgcagcctctggattc
accttcagtaggtatggcatgagctgggtccgccaggctccagggaagaggctggagtgggtctcaaccattagtggcgga
ggcacctacatatactacccagactcagtgaagggccgattcaccatctccagagacaacgccaagaacaccctgtatctg
caaatgaacagcctgagagccgaggacacggctgtgtattactgtaccagagataactatggccgcaactatgattatggc
atggattattggggccagggcaccctggtgaccgtgagcagcggcggtggcggatccggcggtggcggatccggcggtggc
ggatccgaaattgtgttgacacagtctccagccaccctgtctttgtctccaggggaaagagccaccctcacctgcagcgcc
accagcagtgttagctacatccactggtaccaacagaggcctggccagagccccaggctcctcatctatagcacctccaac
ctggccagcggcatcccagccaggttcagtggcagtgggtctgggagcgactacactctcaccatcagcagcctagagcct
gaagattttgcagtttattactgtcagcagcgtagcagctcccctttcacctttggcagcggcaccaaagtggaaattaaa
(amino acids)
(SEQ ID NO: 392)
EVQLVESGGGLVKPGGSLRLSCAASGFTFSRYGMSWVRQAPGKRLEWVSTISGGGTYIYYPDSVKGRFTISRDNAKNTLY
LQMNSLRAEDTAVYYCTRDNYGRNYDYGMDYWGQGTLVTVSSGGGGSGGGGSGGGGSEIVLTQSPATLSLSPGERATLTC
SATSSVSYIHWYQQRPGQSPRLLIYSTSNLASGIPARFSGSGSGSDYTLTISSLEPEDFAVYYCQQRSSSPFTFGSGTKV
EIK
HumanizeE6 scFV (VL-VH) sequence:
(DNA)
(SEQ ID NO: 393)
gaaattgtgttgacacagtctccagccaccctgtctttgtctccaggggaaagagccaccctcacctgcagcgccaccagc
agtgttagctacatccactggtaccaacagaggcctggccagagccccaggctcctcatctatagcacctccaacctggcc
agcggcatcccagccaggttcagtggcagtgggtctgggagcgactacactctcaccatcagcagcctagagcctgaagat
tttgcagtttattactgtcagcagcgtagcagctcccctttcacctttggcagcggcaccaaagtggaaattaaaggcggt
ggcggatccggcggtggcggatccggcggtggcggatccgaggtgcagctggtggagtctgggggaggcctggtcaagcct
ggggggtccctgagactctcctgtgcagcctctggattcaccttcagtaggtatggcatgagctgggtccgccaggctcca
gggaagaggctggagtgggtctcaaccattagtggcggaggcacctacatatactacccagactcagtgaagggccgattc
accatctccagagacaacgccaagaacaccctgtatctgcaaatgaacagcctgagagccgaggacacggctgtgtattac
tgtaccagagataactatggccgcaactatgattatggcatggattattggggccagggcaccctggtgaccgtgagcagc
(amino acids)
(SEQ ID NO: 394)
EIVLTQSPATLSLSPGERATLTCSATSSVSYIHWYQQRPGQSPRLLIYSTSNLASGIPARFSGSGSGSDYTLTISSLEPE
DFAVYYCQQRSSSPFTFGSGTKVEIKGGGGSGGGGSGGGGSEVQLVESGGGLVKPGGSLRLSCAASGFTFSRYGMSWVRQ
APGKRLEWVSTISGGGTYIYYPDSVKGRFTISRDNAKNTLYLQMNSLRAEDTAVYYCTRDNYGRNYDYGMDYWGQGTLVT
VSS
HumanizeC2 scFV (VH-VL) sequence:
(DNA)
(SEQ ID NO: 395)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaact
(amino acids)
(SEQ ID NO: 396)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRT
HumanizeE6 scFV (VL-VH) sequence:
(DNA)
(SEQ ID NO: 397)
gacattgtgctgacccagtctccagcctccttggccgtgtctccaggacagagggccaccatcacctgcagagccagtaag
agtgtcagtaccagcggatactcctacatgcactggtatcagcagaaaccaggacaacctcctaaactcctgatttacctg
gcatccaatctggagagcggggtcccagccaggttcagcggcagtgggtctgggaccgatttcaccctcacaattaatcct
gtggaagctaatgatactgcaaattattactgtcagcacagtagggagctgcctttcacattcggcggagggaccaaggtg
gagatcaaacgaactggcggtggcggatccggcggtggcggatccggcggtggcggatccgaggtgcagctggtggagtct
gggggaggcctggtcaagcctggggggtccctgagactctcctgtgcagcctctggattcaccttcagtggctatgccatg
agctgggtccgccaggctccagggaaggggctggagtgggtctcaaccattagtagtggcggaacctacatatactacccc
gactcagtgaagggccgattcaccatctccagagacaacgccaagaactcactgtatctgcaaatgaacagcctgagagcc
gaggacacggccgtgtattactgtgcgagacttgggggggataattactacgaatacttcgatgtctggggcaaagggacc
acggtcaccgtctcctcc
(amino acids)
(SEQ ID NO: 398)
DIVLTQSPASLAVSPGQRATITCRASKSVSTSGYSYMHWYQQKPGQPPKLLIYLASNLESGVPARFSGSGSGTDFTLTIN
PVEANDTANYYCQHSRELPFTFGGGTKVEIKRTGGGGSGGGGSGGGGSEVQLVESGGGLVKPGGSLRLSCAASGFTFSGY
AMSWVRQAPGKGLEWVSTISSGGTYIYYPDSVKGRFTISRDNAKNSLYLQMNSLRAEDTAVYYCARLGGDNYYEYFDVWG
KGTTVTVSS
G₄S₁ linker sequence:
(DNA)
(SEQ ID NO: 399)
ggcggtggcggatcc
(amino acids)
(SEQ ID NO: 400)
GGGGS
[G₄S₁]x3 linker sequence:
(DNA)
(SEQ ID NO: 401)
ggcggtggcggatccggcggtggcggatccggcggtggcggatcc
(amino acids)
(SEQ ID NO: 402)
GGGGSGGGGSGGGGS
8 aa GS linker sequence:
(DNA)
(SEQ ID NO: 403)
ggcggttccggcggtggatccgga
(amino acids)
(SEQ ID NO: 404)
GGSGGGSG
12 aa GS linker sequence:
(DNA)
(SEQ ID NO: 405)
ggcggttccggcggtggatccggcggtggcggatccgga
(amino acids)
(SEQ ID NO: 406)
GGSGGGSGGGSG
13 aa GS linker sequence:
(DNA)
(SEQ ID NO: 407)
ggcggtggatccggcggtggcggatccggcggtggatcc
(amino acids)
(SEQ ID NO: 408)
GGGSGGGGSGGGS
22 aa GS linker sequence:
(DNA)
(SEQ ID NO: 409)
ggcggtggaagcggcggtggcggatccggcagcggcggaagcggcggtggcggatccggcggtgga
(amino acids)
(SEQ ID NO: 4110)
GGGSGGGGSGSGGSGGGGSGGG
24 aa GS linker sequence:
(DNA)
(SEQ ID NO: 411)
ggcggttccggcggtggatccggcggtggcggatccggaggcggttccggcggtggatccggcggtggcggatccgga
(amino acids)
(SEQ ID NO: 412)
GGSGGGSGGGSGGGSGGGSGGGSG
Mouse C3 Heavy chain variable region sequence:
(DNA)
(SEQ ID NO: 413)
caggtccagctgcagcagtctgggcctgagctggtgaggcctggggtctcagtgaagatttcctgcaagggttccggctac
agattcactgattatgctatgaactgggtgaagcagagtcatgcaaagagtctagagtggattggagttattagtactttc
tctggtaatacaaacttcaaccagaagtttaagggcaaggccacaatgactgtagacaaatcctccagcacagcctatatg
gaacttgccagattgacatctgaggattctgccatgtattactgtgcaagatcggattactacggcccatactttgactac
tggggccaaggcaccactctcacagtctcctca
(amino acids)
(SEQ ID NO: 414)
QVQLQQSGPELVRPGVSVKISCKGSGYRFTDYAMNWVKQSHAKSLEWIGVISTFSGNTNFNQKFKGKATMTVDKSSSTAYM
ELARLTSEDSAMYYCARSDYYGPYFDYWGQGTTLTVSS
Mouse C3 heavy chain variable framework region 1 (FWR1) sequence:
(DNA)
(SEQ ID NO: 415)
caggtccagctgcagcagtctgggcctgagctggtgaggcctggggtctcagtgaagatttcctgcaagggttccggcta
cagattcact
(amino acids)
(SEQ ID NO: 416)
QVQLQQSGPELVRPGVSVKISCKGSGYRFT
Mouse C3 heavy chain variable complementarity determining regions 1 (CDR1)
sequence:
(DNA)
(SEQ ID NO: 417)
gattatgctatgaac
(amino acids)
(SEQ ID NO: 418)
DYAMN
Mouse C3 heavy chain variable framework region 2 (FWR2) sequence:
(DNA)
(SEQ ID NO: 419)
tgggtgaagcagagtcatgcaaagagtctagagtggattgga
(amino acids)
(SEQ ID NO: 420)
WVKQSHAKSLEWIG
Mouse C3 heavy chain variable complementarity determining regions 2 (CDR2)
sequence:
(DNA)
(SEQ ID NO: 421)
gttattagtactttctctggtaatacaaacttcaaccagaagtttaagggc
(amino acids)
(SEQ ID NO: 422)
VISTFSGNTNFNQKFKG
Mouse C3 heavy chain variable framework region 3 (FWR3) acid sequence:
(DNA)
(SEQ ID NO: 423)
aaggccacaatgactgtagacaaatcctccagcacagcctatatggaacttgccagattgacatctgaggattctgccat
gtattactgtgcaaga
(amino acids)
(SEQ ID NO: 424)
KATMTVDKSSSTAYMELARLTSEDSAMYYCAR
Mouse C3 heavy chain variable complementarity determining regions 3 (CDR3)
sequence:
(DNA)
(SEQ ID NO: 425)
tcggattactacggcccatactttgactac
(amino acids)
(SEQ ID NO: 426)
SDYYGPYFDY
IGHV1-18*04 heavy chain variable region sequence:
(DNA)
(SEQ ID NO: 427)
caggttcagctggtgcagtctggagctgaggtgaagaagcctggggcctcagtgaaggtctcctgcaaggcttctggttac
acctttaccagctacggtatcagctgggtgcgacaggcccctggacaagggcttgagtggatgggatggatcagcgcttac
aatggtaacacaaactatgcacagaagctccagggcagagtcaccatgaccacagacacatccacgagcacagcctacatg
gagctgaggagcctgagatctgacgacacggccgtgtattactgtgcgagaga
(amino acids)
(SEQ ID NO: 428)
QVQLVQSGAEVKKPGASVKVSCKASGYTFTSYGISWVRQAPGQGLEWMGWISAYNGNTNYAQKLQGRVTMTTDTSTSTAY
MELRSLRSDDTAVYYCAR
IGHV1-18*04 heavy chain variable framework region 1 (FWR1) sequence:
(DNA)
(SEQ ID NO: 429)
caggttcagctggtgcagtctggagctgaggtgaagaagcctggggcctcagtgaaggtctcctgcaaggcttctggtta
cacctttacc
(amino acids)
(SEQ ID NO: 430)
QVQLVQSGAEVKKPGASVKVSCKASGYTFT
IGHV1-18*04 heavy chain variable complementarity determining regions 1 (CDR1)
sequence:
(DNA)
(SEQ ID NO: 431)
agctacggtatcagc
(amino acids)
(SEQ ID NO: 432)
SYGIS
IGHV1-18*04 heavy chain variable framework region 2 (FWR2) sequence:
(DNA)
(SEQ ID NO: 433)
tgggtgcgacaggcccctggacaagggcttgagtggatggga
(amino acids)
(SEQ ID NO: 434)
WVRQAPGQGLEWMG
IGHV1-18*04 heavy chain variable complementarity determining regions 2 (CDR2)
sequence:
(DNA)
(SEQ ID NO: 435)
tggatcagcgcttacaatggtaacacaaactatgcacagaagctccagggc
(amino acids)
(SEQ ID NO: 436)
WISAYNGNTNYAQKLQG
IGHV1-18*04 heavy chain variable framework region 3 (FWR3) sequence:
(DNA)
(SEQ ID NO: 437)
agagtcaccatgaccacagacacatccacgagcacagcctacatggagctgaggagcctgagatctgacgacacggccgt
gtattactgtgcgaga
(amino acids)
(SEQ ID NO: 438)
RVTMTTDTSTSTAYMELRSLRSDDTAVYYCAR
Humanized C3 heavy chain variable region sequence:
(DNA)
(SEQ ID NO: 439)
caggttcagctggtgcagtctggagctgaggtgaagaagcctggggcctcagtgaaggtctcctgcaaggcttctggtta
cacctttaccgactacgccatgaactgggtgcgacaggcccctggacaagggcttgagtggatgggagtgatcagcacct
tcagcggtaacacaaacttcaaccagaagttcaagggcagagtcaccatgaccacagacacatccacgagcacagcctac
atggagctgaggagcctgagatctgacgacacggccgtgtattactgtgcgagaagcgactactacggcccatacttcga
ctactggggccagggcaccaccctgaccgtgtccagc
(amino acids)
(SEQ ID NO: 440)
QVQLVQSGAEVKKPGASVKVSCKASGYTFTDYAMNWVRQAPGQGLEWMGVISTFSGNTNFNQKFKGRVTMTTDTSTSTAY
MELRSLRSDDTAVYYCARSDYYGPYFDYWGQGTTLTVSS
Humanized C3 heavy chain variable framework region 1 (FWR1) acid sequence:
(DNA)
(SEQ ID NO: 441)
caggttcagctggtgcagtctggagctgaggtgaagaagcctggggcctcagtgaaggtctcctgcaaggcttctggtta
cacctttacc
(amino acids)
(SEQ ID NO: 442)
QVQLVQSGAEVKKPGASVKVSCKASGYTFT
Humanized C3 heavy chain variable complementarity determining regions 1 (CDR1)
sequence:
(DNA)
(SEQ ID NO: 443)
gactacgccatgaac
(amino acids)
(SEQ ID NO: 444)
DYAMN
Humanized C3 heavy chain variable framework region 2 (FWR2) acid sequence:
(DNA)
(SEQ ID NO: 445)
tgggtgcgacaggcccctggacaagggcttgagtggatggga
(amino acids)
(SEQ ID NO: 446)
WVRQAPGQGLEWMG
Humanized C3 heavy chain variable complementarity determining regions 2 (CDR2)
sequence:
(DNA)
(SEQ ID NO: 447)
gtgatcagcaccttcagcggtaacacaaacttcaaccagaagttcaagggc
(amino acids)
(SEQ ID NO: 448)
VISTFSGNTNFNQKFKG
Humanized C3 heavy chain variable framework region 3 (FWR3) acid sequence:
(DNA)
(SEQ ID NO: 449)
agagtcaccatgaccacagacacatccacgagcacagcctacatggagctgaggagcctgagatctgacgacacggccgtg
tattactgtgcgaga
(amino acids)
(SEQ ID NO: 450)
RVTMTTDTSTSTAYMELRSLRSDDTAVYYCAR
Humanized C3 heavy chain variable complementarity determining regions 3 (CDR3)
sequence:
(DNA)
(SEQ ID NO: 451)
agcgactactacggcccatacttcgactac
(amino acids)
(SEQ ID NO: 452)
SDYYGPYFDY
Humanized C3 IgG1 heavy chain sequence
(DNA)
(SEQ ID NO: 453)
caggttcagctggtgcagtctggagctgaggtgaagaagcctggggcctcagtgaaggtctcctgcaaggcttctggttac
acctttaccgactacgccatgaactgggtgcgacaggcccctggacaagggcttgagtggatgggagtgatcagcaccttc
agcggtaacacaaacttcaaccagaagttcaagggcagagtcaccatgaccacagacacatccacgagcacagcctacatg
gagctgaggagcctgagatctgacgacacggccgtgtattactgtgcgagaagcgactactacggcccatacttcgactac
tggggccagggcaccaccctgaccgtgtccagcgctagcaccaagggcccatcggtcttccccctggcaccctcctccaag
agcacctctgggggcacagcggccctgggctgcctggtcaaggactacttccccgaaccggtgacggtgtcgtggaactca
ggcgccctgaccagcggcgtgcacaccttcccggctgtcctacagtcctcaggactctactccctcagcagcgtggtgaca
gtgccctccagcagcttgggcacccagacctacatctgcaacgtgaatcacaagcccagcaacaccaaggtggacaagaaa
gttgagcccaaatcttgtgacaaaactcacacatgcccaccgtgcccagcacctgaactcctggggggaccgtcagtcttc
ctcttccccccaaaacccaaggacaccctcatgatctcccggacccctgaggtcacatgcgtggtggtggacgtgagccac
gaagaccctgaggtcaagttcaactggtacgtggacggcgtggaggtgcataatgccaagacaaagccgcgggaggagcag
tacaacagcacgtaccgtgtggtcagcgtcctcaccgtcctgcaccaggactggctgaatggcaaggagtacaagtgcaag
gtctccaacaaagccctcccagcccccatcgagaaaaccatctccaaagccaaagggcagccccgagaaccacaggtgtac
accctgcccccatcccgggaggagatgaccaagaaccaggtcagcctgacctgcctggtcaaaggcttctatcccagcgac
atcgccgtggagtgggagagcaatgggcagccggagaacaactacaagaccacgcctcccgtgctggactccgacggctcc
ttcttcctctacagcaagctcaccgtggacaagagcaggtggcagcaggggaacgtcttctcatgctccgtgatgcatgag
gctctgcacaaccactacacgcagaagagcctctccctgtctccgggtaaatgataa
(amino acids)
(SEQ ID NO: 454)
QVQLVQSGAEVKKPGASVKVSCKASGYTFTDYAMNWVRQAPGQGLEWMGVISTFSGNTNFNQKFKGRVTMTTDTSTSTAY
MELRSLRSDDTAVYYCARSDYYGPYFDYWGQGTTLTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKKVEPKSCDKTHTCPPCPAPELLGGP
SVFLFPPKPKDTLMISRTPEVTCVVVDVSHEDPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK**
Humanized C3 IgG2 heavy chain sequence
(DNA)
(SEQ ID NO: 455)
caggttcagctggtgcagtctggagctgaggtgaagaagcctggggcctcagtgaaggtctcctgcaaggcttctggttac
acctttaccgactacgccatgaactgggtgcgacaggcccctggacaagggcttgagtggatgggagtgatcagcaccttc
agcggtaacacaaacttcaaccagaagttcaagggcagagtcaccatgaccacagacacatccacgagcacagcctacatg
gagctgaggagcctgagatctgacgacacggccgtgtattactgtgcgagaagcgactactacggcccatacttcgactac
tggggccagggcaccaccctgaccgtgtccagcgcctccaccaagggcccatcggtcttccccctggcgccctgctccagg
agcacctccgagagcacagccgccctgggctgcctggtcaaggactacttccccgaaccggtgacggtgtcgtggaactca
ggcgctctgaccagcggcgtgcacaccttcccagctgtcctacagtcctcaggactctactccctcagcagcgtggtgacc
gtgccctccagcaacttcggcacccagacctacacctgcaacgtagatcacaagcccagcaacaccaaggtggacaagaca
gttgagcgcaaatgttgtgtcgagtgcccaccgtgcccagcaccacctgtggcaggaccgtcagtcttcctcttcccccca
aaacccaaggacaccctcatgatctcccggacccctgaggtcacgtgcgtggtggtggacgtgagccacgaagaccccgag
gtccagttcaactggtacgtggacggcgtggaggtgcataatgccaagacaaagccacgggaggagcagttcaacagcacg
ttccgtgtggtcagcgtcctcaccgttgtgcaccaggactggctgaacggcaaggagtacaagtgcaaggtctccaacaaa
ggcctcccagcccccatcgagaaaaccatctccaaaaccaaagggcagccccgagaaccacaggtgtacaccctgccccca
tcccgggaggagatgaccaagaaccaggtcagcctgacctgcctggtcaaaggcttctaccccagcgacatcgccgtggag
tgggagagcaatgggcagccggagaacaactacaagaccacacctcccatgctggactccgacggctccttcttcctctac
agcaagctcaccgtggacaagagcaggtggcagcaggggaacgtcttctcatgctccgtgatgcatgaggctctgcacaac
cactacacgcagaagagcctctccctgtctccgggtaaatagtaa
(amino acids)
(SEQ ID NO: 456)
QVQLVQSGAEVKKPGASVKVSCKASGYTFTDYAMNWVRQAPGQGLEWMGVISTFSGNTNFNQKFKGRVTMTTDTSTSTAY
MELRSLRSDDTAVYYCARSDYYGPYFDYWGQGTTLTVSSASTKGPSVFPLAPCSRSTSESTAALGCLVKDYFPEPVTVSW
NSGALTSGVHTFPAVLQSSGLYSLSSVVTVPSSNFGTQTYTCNVDHKPSNTKVDKTVERKCCVECPPCPAPPVAGPSVFL
FPPKPKDTLMISRTPEVTCVVVDVSHEDPEVQFNWYVDGVEVHNAKTKPREEQFNSTFRVVSVLTVVHQDWLNGKEYKCK
VSNKGLPAPIEKTISKTKGQPREPQVYTLPPSREEMTKNQVSLTCLVKGFYPSDIAVEWESNGQPENNYKTTPPMLDSDG
SFFLYSKLTVDKSRWQQGNVFSCSVMHEALHNHYTQKSLSLSPGK**
Humanized C3 heavy chain IgG1 gBLOCK sequence:
(DNA)
(SEQ ID NO: 457)
tgctctgggttccaggttccactggtgacgcggcccagccggcccaggttcagctggtgcagtctggagctgaggtgaaga
agcctggggcctcagtgaaggtctcctgcaaggcttctggttacacctttaccgactacgccatgaactgggtgcgacagg
cccctggacaagggcttgagtggatgggagtgatcagcaccttcagcggtaacacaaacttcaaccagaagttcaagggca
gagtcaccatgaccacagacacatccacgagcacagcctacatggagctgaggagcctgagatctgacgacacggccgtgt
attactgtgcgagaagcgactactacggcccatacttcgactactggggccagggcaccaccctgaccgtgtccagcgcta
gcaccaagggcccatcggtcttccccctggcaccctcctccaagagcacctctgggggcacagcggccctgggctgcctgg
tcaaggactacttccccgaaccggtgacggtgtcgtggaactcaggcgccctgaccagc
Mouse C3 Light Chain variable region sequence:
(DNA)
(SEQ ID NO: 458)
gatgttttgatgacccaaactccactctccctgcctgtcagtcttggagatcaagcctccatctcttgcagatctagtcag
accattgtacatagtaatggaaacacctatttagaatggtacctgcagaaaccaggccagtctccaaagctcctgatctac
aaagtttccaaccgattttctggggtcccagacaggttcagtggcagtggatcagggacagatttcacactcaagatcaac
agagtggaggctgaggatctgggagtttattactgctttcaaggttcacatgttccattcacgttcggctcggggacaaag
ttggaaataaaa
(amino acids)
(SEQ ID NO: 459)
DVLMTQTPLSLPVSLGDQASISCRSSQTIVHSNGNTYLEWYLQKPGQSPKLLIYKVSNRFSGVPDRFSGSGSGTDFTLKIN
RVEAEDLGVYYCFQGSHVPFTFGSGTKLEIK
Mouse C3 light chain variable framework region 1 (FWR1) sequence:
(DNA)
(SEQ ID NO: 460)
gatgttttgatgacccaaactccactctccctgcctgtcagtcttggagatcaagcctccatctcttgc
(amino acids)
(SEQ ID NO: 461)
DVLMTQTPLSLPVSLGDQASISC
Mouse C3 light chain variable complementarity determining regions 1 (CDR1)
sequence:
(DNA)
(SEQ ID NO: 462)
agatctagtcagaccattgtacatagtaatggaaacacctatttagaa
(amino acids)
(SEQ ID NO: 463)
RSSQTIVHSNGNTYLE
Mouse C3 light chain variable framework region 2 (FWR2) sequence:
(DNA)
(SEQ ID NO: 464)
tggtacctgcagaaaccaggccagtctccaaagctcctgatctac
(amino acids)
(SEQ ID NO: 465)
WYLQKPGQSPKLLIY
Mouse C3 light chain variable complementarity determining regions 2 (CDR2)
sequence:
(DNA)
(SEQ ID NO: 466)
aaagtttccaaccgattttct
(amino acids)
(SEQ ID NO: 467)
KVSNRFS
Mouse C3 light chain variable framework region 3 (FWR3) sequence:
(DNA)
(SEQ ID NO: 468)
ggggtcccagacaggttcagtggcagtggatcagggacagatttcacactcaagatcaacagagtggaggctgaggatct
gggagtttattactgc
(amino acids)
(SEQ ID NO: 469)
GVPDRFSGSGSGTDFTLKINRVEAEDLGVYYC
Mouse C3 light chain variable complementarity determining regions 3 (CDR3)
sequence:
(DNA)
(SEQ ID NO: 470)
tttcaaggttcacatgttccattcacg
(amino acids)
(SEQ ID NO: 471)
FQGSHVPFT
IGKV2-29*03 light chain variable region sequence:
(DNA)
(SEQ ID NO: 472)
gatattgtgatgacccagactccactctctctgtccgtcacccctggacagccggcctccatctcctgcaagtctagtca
gagcctcctgcatagtgatggaaagacctatttgtattggtacctgcagaagccaggccagtctccacagctcctgatct
atgaagtttccagccggttctctggagtgccagataggttcagtggcagcgggtcagggacagatttcacactgaaaatc
agccgggtggaggctgaggatgttggggtttattactgcatgcaaggtatacaccttcct
(amino acids)
(SEQ ID NO: 473)
DIVMTQTPLSLSVTPGQPASISCKSSQSLLHSDGKTYLYWYLQKPGQSPQLLIYEVSSRFSGVPDRFSGSGSGTDFTLKI
SRVEAEDVGVYYCMQGIHLP
IGKV2-29*03 light chain variable framework region 1 (FWR1) acid sequence:
(DNA)
(SEQ ID NO: 474)
gatattgtgatgacccagactccactctctctgtccgtcacccctggacagccggcctccatctcctgc
(amino acids)
(SEQ ID NO: 475)
DIVMTQTPLSLSVTPGQPASISC
IGKV2-29*03 light chain variable complementarity determining regions 1 (CDR1)
sequence:
(DNA)
(SEQ ID NO: 476)
aagtctagtcagagcctcctgcatagtgatggaaagacctatttgtat
(amino acids)
(SEQ ID NO: 477)
KSSQSLLHSDGKTYLY
IGKV2-29*03 light chain variable framework region 2 (FWR2) sequence:
(DNA)
(SEQ ID NO: 478)
tggtacctgcagaagccaggccagtctccacagctcctgatctat
(amino acids)
(SEQ ID NO: 479)
WYLQKPGQSPQLLIY
IGKV2-29*03 light chain variable complementarity determining regions 2 (CDR2)
sequence:
(DNA)
(SEQ ID NO: 480)
gaagtttccagccggttc
(amino acids)
(SEQ ID NO: 481)
EVSSRFS
IGKV2-29*03 light chain variable framework region 3 (FWR3) sequence:
(DNA)
(SEQ ID NO: 482)
ggagtgccagataggttcagtggcagcgggtcagggacagatttcacactgaaaatcagccgggtggaggctgaggatgt
tggggtttattactgc
(amino acids)
(SEQ ID NO: 483)
GVPDRFSGSGSGTDFTLKISRVEAEDVGVYYC
IGKV2-29*03 light chain variable complementarity determining regions3 (CDR3)
sequence:
(DNA)
(SEQ ID NO: 484)
atgcaaggtatacaccttcct
(amino acids)
(SEQ ID NO: 485)
MQGIHLP
Humanized C3 light chain variable region sequence:
(DNA)
(SEQ ID NO: 486)
gatattgtgatgacccagactccactctctctgtccgtcacccctggacagccggcctccatctcctgcaggtctagtcag
accattgtccatagtaatggaaacacctatttggagtggtacctgcagaagccaggccagtctccacagctcctgatctat
aaggtttccaaccggttctctggagtgccagataggttcagtggcagcgggtcagggacagatttcacactgaaaatcagc
cgggtggaggctgaggatgttggggtttattactgcttccaaggtagccacgtgcctttcaccttcggcggagggaccaag
gtggagatcaaacgaact
(amino acids)
(SEQ ID NO: 487)
DIVMTQTPLSLSVTPGQPASISCRSSQTIVHSNGNTYLEWYLQKPGQSPQLLIYKVSNRFSGVPDRFSGSGSGTDFTLKI
SRVEAEDVGVYYCFQGSHVPFTFGGGTKVEIKRT
Humanized C3 light chain variable framework region 1 (FWR1) acid sequence:
(DNA)
(SEQ ID NO: 488)
gatattgtgatgacccagactccactctctctgtccgtcacccctggacagccggcctccatctcctgc
(amino acids)
(SEQ ID NO: 489)
DIVMTQTPLSLSVTPGQPASISC
Humanized C3 light chain variable complementarity determining regions 1 (CDR1)
sequence:
(DNA)
(SEQ ID NO: 490)
ggtctagtcagaccattgtccatagtaatggaaacacctatttggag
(amino acids)
(SEQ ID NO: 491)
RSSQTIVHSNGNTYLE
Humanized C3 light chain variable framework region 2 (FWR2) acid sequence:
(DNA)
(SEQ ID NO: 492)
tggtacctgcagaagccaggccagtctccacagctcctgatctat
(amino acids)
(SEQ ID NO: 493)
WYLQKPGQSPQLLIY
Humanized C3 light chain variable complementarity determining regions 2 (CDR2)
sequence:
(DNA)
(SEQ ID NO: 494)
aaggtttccaaccggttctct
(amino acids)
(SEQ ID NO: 495)
KVSNRFS
Humanized C3 light chain variable framework region 3 (FWR3) acid sequence:
(DNA)
(SEQ ID NO: 496)
ggagtgccagataggttcagtggcagcgggtcagggacagatttcacactgaaaatcagccgggtggaggctgaggatgtt
ggggtttattactgc
(amino acids)
(SEQ ID NO: 497)
GVPDRFSGSGSGTDFTLKISRVEAEDVGVYYC
Humanized C3 light chain variable complementarity determining regions 3 (CDR3)
sequence:
(DNA)
(SEQ ID NO: 498)
ttccaaggtagccacgtgcctttcacc
(amino acids)
(SEQ ID NO: 499)
FQGSHVPFT
Humanized C3 lambda light chain sequence
(DNA)
(SEQ ID NO: 500)
gatattgtgatgacccagactccactctctctgtccgtcacccctggacagccggcctccatctcctgcaggtctagtcag
accattgtccatagtaatggaaacacctatttggagtggtacctgcagaagccaggccagtctccacagctcctgatctat
aaggtttccaaccggttctctggagtgccagataggttcagtggcagcgggtcagggacagatttcacactgaaaatcagc
cgggtggaggctgaggatgttggggtttattactgcttccaaggtagccacgtgcctttcaccttcggcggagggaccaag
gtggagatcaaacgaactggtcagcccaaggctgccccctcggtcactctgttcccgccctcctctgaggagcttcaagcc
aacaaggccacactggtgtgtctcataagtgacttctacccgggagccgtgacagtggcctggaaggcagatagcagcccc
gtcaaggcgggagtggagaccaccacaccctccaaacaaagcaacaacaagtacgcggccagcagctatctgagcctgacg
cctgagcagtggaagtcccacagaagctacagctgccaggtcacgcatgaagggagcaccgtggagaagacagtggcccct
acagaatgttcatagtaa
(amino acids)
(SEQ ID NO: 501)
DIVMTQTPLSLSVTPGQPASISCRSSQTIVHSNGNTYLEWYLQKPGQSPQLLIYKVSNRFSGVPDRFSGSGSGTDFTLKI
SRVEAEDVGVYYCFQGSHVPFTFGGGTKVEIKRTGQPKAAPSVTLFPPSSEELQANKATLVCLISDFYPGAVTVAWKADS
SPVKAGVETTTPSKQSNNKYAASSYLSLTPEQWKSHRSYSCQVTHEGSTVEKTVAPTECS**
Humanized C3 Kappa light chain
(DNA)
(SEQ ID NO: 502)
gatattgtgatgacccagactccactctctctgtccgtcacccctggacagccggcctccatctcctgcaggtctagtcag
accattgtccatagtaatggaaacacctatttggagtggtacctgcagaagccaggccagtctccacagctcctgatctat
aaggtttccaaccggttctctggagtgccagataggttcagtggcagcgggtcagggacagatttcacactgaaaatcagc
cgggtggaggctgaggatgttggggtttattactgcttccaaggtagccacgtgcctttcaccttcggcggagggaccaag
gtggagatcaaacgaactacggtggctgcaccatctgtcttcatcttcccgccatctgatgagcagttgaaatctggaact
gcctctgttgtgtgcctgctgaataacttctatcccagagaggccaaagtacagtggaaggtggataacgccctccaatcg
ggtaactcccaggagagtgtcacagagcaggacagcaaggacagcacctacagcctcagcagcaccctgacgctgagcaaa
gcagactacgagaaacacaaagtctacgcctgcgaagtcacccatcagggcctgagctcgcccgtcacaaagagcttcaac
aggggagagtgttagtaa
(amino acids)
(SEQ ID NO: 503)
DIVMTQTPLSLSVTPGQPASISCRSSQTIVHSNGNTYLEWYLQKPGQSPQLLIYKVSNRFSGVPDRFSGSGSGTDFTLKI
SRVEAEDVGVYYCFQGSHVPFTFGGGTKVEIKRTTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC**
Humanized C3 Kappa light gBLOCK sequence:
(DNA)
(SEQ ID NO: 504)
agctggctaggtaagcttggtaccgagctcggatccacgccaccatggagacagacacactcctgctatgggtactgctgc
tctgggttccaggttccactggtgacgatattgtgatgacccagactccactctctctgtccgtcacccctggacagccgg
cctccatctcctgcaggtctagtcagaccattgtccatagtaatggaaacacctatttggagtggtacctgcagaagccag
gccagtctccacagctcctgatctataaggtttccaaccggttctctggagtgccagataggttcagtggcagcgggtcag
ggacagatttcacactgaaaatcagccgggtggaggctgaggatgttggggtttattactgcttccaaggtagccacgtgc
ctttcaccttcggcggagggaccaaggtggagatcaaacgaactacggtggctgcaccatctgtcttcatcttcccgccat
ctgatgagcagttgaaatctggaactgcctctgttgtgtgcctgctgaataacttctatcccagagaggccaaagtacagt
ggaaggtggataacgccctccaatcgggtaactcccaggagagtgtcacagagcaggacagcaaggacagcacctacagcc
tcagcagcaccctgacgctgagcaaagcagactacgagaaacacaaagtctacgcctgcgaagtcacccatcagggcctga
gctcgcccgtcacaaagagcttcaacaggggagagtgttagtaagtttaaacccgctgatcagcctcgactgtgccttcta
gttgc
Mouse C8 heavy chain variable region sequence
(DNA)
(SEQ ID NO: 505)
gaagtgatggtcgtggaaagcggcggtggtctggtaaagccggggggatcccttaagctttcttgcgccgcatccgggttc
acgttctccggctatgccatgtcctgggtccgacagactcccgaaaagcgcttggaatgggtggccactatctcctccggg
gggacgtacatctactaccccgacagtgtgaaaggaagatttacaatatctcgcgacaacgcaaaaaataccttgtatctt
caaatgagctccctgcggtcagaggacactgccatgtactattgcgcccgcctgggcggcgacaattactatgagtat
(amino acids)
(SEQ ID NO: 506)
EVMVVESGGGLVKPGGSLKLSCAASGFTFSGYAMSWVRQTPEKRLEWVATISSGGTYIYYPDSVKGRFTISRDNAKNTLYL
QMSSLRSEDTAMYYCARLGGDNYYEY
Mouse C8 heavy chain variable complementarity determining region 1 (CDR1)
sequence:
(DNA)
(SEQ ID NO: 507)
ggctatgccatgtcc
(amino acids)
(SEQ ID NO: 508)
GYAMS
Mouse C8 heavy chain variable complementarity determining region 2 (CDR2)
sequence:
(DNA)
(SEQ ID NO: 509)
actatctcctccggggggacgtacatctactaccccgacagtgtgaaagga
(amino acids)
(SEQ ID NO: 510)
TISSGGTYIYYPDSVKG
Mouse C8 heavy chain variable complementarity determining region 3 (CDR3)
sequence:
(DNA)
(SEQ ID NO: 511)
ctgggcggcgacaattactatgagtat
(amino acids)
(SEQ ID NO: 512)
LGGDNYYEY
IGHV3-21*04 heavy chain variable region sequence:
(DNA)
(SEQ ID NO: 513)
gaggtgcagctggtggagtctgggggaggcctggtcaagcctggggggtccctgagactctcctgtgcagcctctggatt
caccttcagtagctatagcatgaactgggtccgccaggctccagggaaggggctggagtgggtctcatccattagtagta
gtagtagttacatatactacgcagactcagtgaagggccgattcaccatctccagagacaacgccaagaactcactgtat
ctgcaaatgaacagcctgagagccgaggacacggccgtgtattactgtgcga
(amino acids)
(SEQ ID NO: 514)
EVQLVESGGGLVKPGGSLRLSCAASGFTFSSYSMNWVRQAPGKGLEWVSSISSSSSYIYYADSVKGRFTISRDNAKNSLY
LQMNSLRAEDTAVYYCAR
IGHV3-21*04 heavy chain variable framework region 1 (FWR1) sequence:
(DNA)
(SEQ ID NO: 515)
gaggtgcagctggtggagtctgggggaggcctggtcaagcctggggggtccctgagactctcctgtgcagcctctggatt
caccttcagt
(amino acids)
(SEQ ID NO: 516)
EVQLVESGGGLVKPGGSLRLSCAASGFTFS
IGHV3-21*04 heavy chain variable complementarity determining regions 1 (CDR1)
sequence:
(DNA)
(SEQ ID NO: 517)
agctatagcatgaac
(amino acids)
(SEQ ID NO: 518)
SYSMN
IGHV3-21*04 heavy chain variable framework region 2 (FWR2) sequence:
(DNA)
(SEQ ID NO: 519)
tgggtccgccaggctccagggaaggggctggagtgggtc
(amino acids)
(SEQ ID NO: 520)
WVRQAPGKGLEWV
IGHV3-21*04 heavy chain variable complementarity determining regions 2 (CDR2)
sequence:
(DNA)
(SEQ ID NO: 521)
tcatccattagtagtagtagtagttacatatactacgcagactcagtgaagggc
(amino acids)
(SEQ ID NO: 522)
SSISSSSSYIYYADSVKG
IGHV3-21*04 heavy chain variable framework region 3 (FWR3) sequence:
(DNA)
(SEQ ID NO: 523)
cgattcaccatctccagagacaacgccaagaactcactgtatctgcaaatgaacagcctgagagccgaggacacggccgt
gtattactgtgcga
(amino acids)
(SEQ ID NO: 524)
RFTISRDNAKNSLYLQMNSLRAEDTAVYYCAR
Humanized C8 heavy chain variable region sequence:
(DNA)
(SEQ ID NO: 525)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccctgactcagtgaagggccgattcaccatctccagagacaacgccaagaactcactgtatctg
caaatgaacagcctgagagccgaggacacggccgtgtattactgtgcgagactgggcggcgataactattatgaatattgg
ggcaaagggaccacggtcaccgtctcctcc
(amino acids)
(SEQ ID NO: 526)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYWGKGTTVTVSS
Humanized C8 heavy chain variable framework region 1 (FWR1) sequence:
(DNA)
(SEQ ID NO: 527)
gaggtgcagctggtggagtctgggggaggcctggtcaagcctggggggtccctgagactctcctgtgcagcctctggattc
accttcagt
(amino acids)
(SEQ ID NO: 528)
EVQLVESGGGLVKPGGSLRLSCAASGFTFS
Humanized C8 heavy chain variable complementarity determining region 1 (CDR1)
sequence:
(DNA)
(SEQ ID NO: 529)
ggctatgccatgagc
(amino acids)
(SEQ ID NO: 530)
GYAMS
Humanized C8 heavy chain variable framework region 2 (FWR2) sequence:
(DNA)
(SEQ ID NO: 531)
tgggtccgccaggctccagggaaggggctggagtgggtctca
(amino acids)
(SEQ ID NO: 532)
WVRQAPGKGLEWVS
Humanized C8 heavy chain variable complementarity determining region 2 (CDR2)
sequence:
(DNA)
(SEQ ID NO: 533)
accattagtagtggcggaacctacatatactaccctgactcagtgaagggc
(amino acids)
(SEQ ID NO: 534)
TISSGGTYIYYPDSVKG
Humanized C8 heavy chain variable framework region 3 (FWR3) sequence:
(DNA)
(SEQ ID NO: 535)
cgattcaccatctccagagacaacgccaagaactcactgtatctgcaaatgaacagcctgagagccgaggacacggccgtg
tattactgtgcgaga
(amino acids)
(SEQ ID NO: 536)
RFTISRDNAKNSLYLQMNSLRAEDTAVYYCAR
Humanized C8 heavy chain variable complementarity determining region 3 (CDR3)
sequence:
(DNA)
(SEQ ID NO: 537)
ctgggcggcgataactattatgaatat
(amino acids)
(SEQ ID NO: 538)
LGGDNYYEY
Humanized C8 IgG1 heavy chain sequence
(DNA)
(SEQ ID NO: 539)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccctgactcagtgaagggccgattcaccatctccagagacaacgccaagaactcactgtatctg
caaatgaacagcctgagagccgaggacacggccgtgtattactgtgcgagactgggcggcgataactattatgaatattgg
ggcaaagggaccacggtcaccgtctcctccgctagcaccaagggcccatcggtcttccccctggcaccctcctccaagagc
acctctgggggcacagcggccctgggctgcctggtcaaggactacttccccgaaccggtgacggtgtcgtggaactcaggc
gccctgaccagcggcgtgcacaccttcccggctgtcctacagtcctcaggactctactccctcagcagcgtggtgacagtg
ccctccagcagcttgggcacccagacctacatctgcaacgtgaatcacaagcccagcaacaccaaggtggacaagaaagtt
gagcccaaatcttgtgacaaaactcacacatgcccaccgtgcccagcacctgaactcctggggggaccgtcagtcttcctc
ttccccccaaaacccaaggacaccctcatgatctcccggacccctgaggtcacatgcgtggtggtggacgtgagccacgaa
gaccctgaggtcaagttcaactggtacgtggacggcgtggaggtgcataatgccaagacaaagccgcgggaggagcagtac
aacagcacgtaccgtgtggtcagcgtcctcaccgtcctgcaccaggactggctgaatggcaaggagtacaagtgcaaggtc
tccaacaaagccctcccagcccccatcgagaaaaccatctccaaagccaaagggcagccccgagaaccacaggtgtacacc
ctgcccccatcccgggaggagatgaccaagaaccaggtcagcctgacctgcctggtcaaaggcttctatcccagcgacatc
gccgtggagtgggagagcaatgggcagccggagaacaactacaagaccacgcctcccgtgctggactccgacggctccttc
ttcctctacagcaagctcaccgtggacaagagcaggtggcagcaggggaacgtcttctcatgctccgtgatgcatgaggct
ctgcacaaccactacacgcagaagagcctctccctgtctccgggtaaatgataa
(amino acids)
(SEQ ID NO: 540)
EVQLVESGGGLVKPGGSLRLSCAASGFTFSGYAMSWVRQAPGKGLEWVSTISSGGTYIYYPDSVKGRFTISRDNAKNSLYL
QMNSLRAEDTAVYYCARLGGDNYYEYWGKGTTVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGFYPSDIAVEWESNGQPENNYKTTPPVLDSDGSF
FLYSKLTVDKSRWQQGNVFSCSVMHEALHNHYTQKSLSLSPGK**
Humanized C8 IgG2 heavy chain sequence
(DNA)
(SEQ ID NO: 541)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccctgactcagtgaagggccgattcaccatctccagagacaacgccaagaactcactgtatctg
caaatgaacagcctgagagccgaggacacggccgtgtattactgtgcgagactgggcggcgataactattatgaatattgg
ggcaaagggaccacggtcaccgtctcctccgcctccaccaagggcccatcggtcttccccctggcgccctgctccaggagc
acctccgagagcacagccgccctgggctgcctggtcaaggactacttccccgaaccggtgacggtgtcgtggaactcaggc
gctctgaccagcggcgtgcacaccttcccagctgtcctacagtcctcaggactctactccctcagcagcgtggtgaccgtg
ccctccagcaacttcggcacccagacctacacctgcaacgtagatcacaagcccagcaacaccaaggtggacaagacagtt
gagcgcaaatgttgtgtcgagtgcccaccgtgcccagcaccacctgtggcaggaccgtcagtcttcctcttccccccaaaa
cccaaggacaccctcatgatctcccggacccctgaggtcacgtgcgtggtggtggacgtgagccacgaagaccccgaggtc
cagttcaactggtacgtggacggcgtggaggtgcataatgccaagacaaagccacgggaggagcagttcaacagcacgttc
cgtgtggtcagcgtcctcaccgttgtgcaccaggactggctgaacggcaaggagtacaagtgcaaggtctccaacaaaggc
ctcccagcccccatcgagaaaaccatctccaaaaccaaagggcagccccgagaaccacaggtgtacaccctgcccccatcc
cgggaggagatgaccaagaaccaggtcagcctgacctgcctggtcaaaggcttctaccccagcgacatcgccgtggagtgg
gagagcaatgggcagccggagaacaactacaagaccacacctcccatgctggactccgacggctccttcttcctctacagc
aagctcaccgtggacaagagcaggtggcagcaggggaacgtcttctcatgctccgtgatgcatgaggctctgcacaaccac
tacacgcagaagagcctctccctgtctccgggtaaatagtaa
(amino acids)
(SEQ ID NO: 542)
EVQLVESGGGLVKPGGSLRLSCAASGFTFSGYAMSWVRQAPGKGLEWVSTISSGGTYIYYPDSVKGRFTISRDNAKNSLYL
QMNSLRAEDTAVYYCARLGGDNYYEYWGKGTTVTVSSASTKGPSVFPLAPCSRSTSESTAALGCLVKDYFPEPVTVSWNSG
ALTSGVHTFPAVLQSSGLYSLSSVVTVPSSNFGTQTYTCNVDHKPSNTKVDKTVERKCCVECPPCPAPPVAGPSVFLFPPK
PKDTLMISRTPEVTCVVVDVSHEDPEVQFNWYVDGVEVHNAKTKPREEQFNSTFRVVSVLTVVHQDWLNGKEYKCKVSNKG
LPAPIEKTISKTKGQPREPQVYTLPPSREEMTKNQVSLTCLVKGFYPSDIAVEWESNGQPENNYKTTPPMLDSDGSFFLYS
KLTVDKSRWQQGNVFSCSVMHEALHNHYTQKSLSLSPGK**
Mouse C8 light chain variable region sequence
(DNA)
(SEQ ID NO: 543)
gacatcgtcattacgcagacccctgccagtcttgccgtttctctgggccagagggccactatcagttacagggcgagtaag
tctgtgagtaccagcggctatagttacatgcattggaaccagcagaaaccgggacagccaccacgcctgcttatttatctg
gtgtctaatcttgagtccggggtgcccgccaggttcagcggcagcggctctgggaccgacttcacactcaacattcatcca
gtggaagaagaggacgctgctacatactactgtcaacacattcgggaactgaccaggagtgaa
(amino acids)
(SEQ ID NO: 544)
DIVITQTPASLAVSLGQRATISYRASKSVSTSGYSYMHWNQQKPGQPPRLLIYLVSNLESGVPARFSGSGSGTDFTLNIHP
VEEEDAATYYCQHIRELTRSE
Mouse C8 light chain variable complementarity determining region 1 (CDR1)
sequence:
(DNA)
(SEQ ID NO: 545)
agggcgagtaagtctgtgagtaccagcggctatagttacatgcat
(amino acids)
(SEQ ID NO: 546)
RASKSVSTSGYSYMH
Mouse C8 light chain variable complementarity determining region 2 (CDR2)
sequence:
(DNA)
(SEQ ID NO: 547)
ctggtgtctaatcttgagtcc
(amino acids)
(SEQ ID NO: 548)
LVSNLES
Mouse C8 light chain variable complementarity determining region 3 (CDR3)
sequence:
(DNA)
(SEQ ID NO: 549)
caacacattcgggaactgaccaggagtgaa
(amino acids)
(SEQ ID NO: 550)
QHIRELTRSE
NCBI germline z00023 light chain variable region sequence:
(DNA)
(SEQ ID NO: 551)
gacatcgtgatgacccagtctccagactccctggctgtgtctctgggcgagagggccaccatcaactgcaagtccagccag
agtgttttatacagctccaacaataagaactacttagcttggtaccagcagaaaccaggacagcctcctaagctgctcatt
tactgggcatctacccgggaatccggggtccctgaccgattcagtggcagcgggtctgggacagatttcactctcaccatc
agcagcctgcaggctgaagatgtggcagtttattactgtcagcaatattatagtactcct
(amino acids)
(SEQ ID NO: 552)
DIVMTQSPDSLAVSLGERATINCKSSQSVLYSSNNKNYLAWYQQKPGQPPKLLIYWASTRESGVPDRFSGSGSGTDFTLT
ISSLQAEDVAVYYCQQYYSTP
NCBI germline z00023 light chain variable framework region 1 (FWR1) acid
sequence:
(DNA)
(SEQ ID NO: 553)
gacatcgtgatgacccagtctccagactccctggctgtgtctctgggcgagagggccaccatcaactgc
(amino acids)
(SEQ ID NO: 554)
DIVMTQSPDSLAVSLGERATINC
NCBI germline z00023 light chain variable complementarity determining regions 1
(CDR1) sequence:
(DNA)
(SEQ ID NO: 555)
aagtccagccagagtgttttatacagctccaacaataagaactacttagct
(amino acids)
(SEQ ID NO: 556)
KSSQSVLYSSNNKNYLA
NCBI germline z00023 light chain variable framework region 2 (FWR2) sequence:
(DNA)
(SEQ ID NO: 557)
tggtaccagcagaaaccaggacagcctcctaagctgctcatttac
(amino acids)
(SEQ ID NO: 558)
WYQQKPGQPPKLLIY
NCBI germline z00023 light chain variable complementarity determining regions 2
(CDR2) sequence:
(DNA)
(SEQ ID NO: 559)
tgggcatctacccgggaatcc
(amino acids)
(SEQ ID NO: 560)
WASTRES
NCBI germline z00023 light chain variable framework region 3 (FWR3) sequence:
(DNA)
(SEQ ID NO: 561)
ggggtccctgaccgattcagtggcagcgggtctgggacagatttcactctcaccatcagcagcctgcaggctgaagatgt
ggcagtttattactgt
(amino acids)
(SEQ ID NO: 562)
GVPDRFSGSGSGTDFTLTISSLQAEDVAVYYC
NCBI germline z00023 light chain variable complementarity determining regions3
(CDR3) sequence:
(DNA)
(SEQ ID NO: 563)
cagcaatattatagtactcct
(amino acids)
(SEQ ID NO: 564)
QQYYSTP
Humanized C8 light chain variable region sequence
(DNA)
(SEQ ID NO: 565)
gacatcgtgatgacccagtctccagactccctggctgtgtctctgggcgagagggccaccatcaactgcagggccagcaag
agtgttagcaccagcggctacagctacatgcactggtaccagcagaaaccaggacagcctcctaagctgctcatttacctg
gtgtctaacctggaatccggggtccctgaccgattcagtggcagcgggtctgggacagatttcactctcaccatcagcagc
ctgcaggctgaagatgtggcagtttattactgtcaacacattcgggaactgaccaggagtgaattcggcggagggaccaag
gtggagatcaaacgaact
(amino acids)
(SEQ ID NO: 566)
DIVMTQSPDSLAVSLGERATINCRASKSVSTSGYSYMHWYQQKPGQPPKLLIYLVSNLESGVPDRFSGSGSGTDFTLTIS
SLQAEDVAVYYCQHIRELTRSEFGGGTKVEIKRT
Humanized C8 light chain variable framework region 1 (FWR1) sequence:
(DNA)
(SEQ ID NO: 567)
gacatcgtgatgacccagtctccagactccctggctgtgtctctgggcgagagggccaccatcaactgc
(amino acids)
(SEQ ID NO: 568)
DIVMTQSPDSLAVSLGERATINC
Humanized C8 light chain variable complementarity determining region 1 (CDR1)
sequence:
(DNA)
(SEQ ID NO: 569)
agggccagcaagagtgttagcaccagcggctacagctacatg
(amino acids)
(SEQ ID NO: 570)
RASKSVSTSGYSYM
Humanized C8 light chain variable framework region 2 (FWR2) sequence:
(DNA)
(SEQ ID NO: 571)
cactggtaccagcagaaaccaggacagcctcctaagctgctcatttac
(amino acids)
(SEQ ID NO: 572)
HWYQQKPGQPPKLLIY
Humanized C8 light chain variable complementarity determining region 2 (CDR2)
sequence:
(DNA)
(SEQ ID NO: 573)
ctggtgtctaacctggaatcc
(amino acids)
(SEQ ID NO: 574)
LVSNLES
Humanized C8 light chain variable framework region 3 (FWR3) sequence:
(DNA)
(SEQ ID NO: 575)
ggggtccctgaccgattcagtggcagcgggtctgggacagatttcactctcaccatcagcagcctgcaggctgaagatgtg
gcagtttattactgt
(amino acids)
(SEQ ID NO: 576)
GVPDRFSGSGSGTDFTLTISSLQAEDVAVYYC
Humanized C8 light chain variable complementarity determining region 3 (CDR3)
sequence:
(DNA)
(SEQ ID NO: 577)
caacacattcgggaactgaccaggagtgaa
(amino acids)
(SEQ ID NO: 578)
QHIRELTRSE
Humanized C8 Lambda light chain sequence
(DNA)
(SEQ ID NO: 579)
gacatcgtgatgacccagtctccagactccctggctgtgtctctgggcgagagggccaccatcaactgcagggccagcaag
agtgttagcaccagcggctacagctacatgcactggtaccagcagaaaccaggacagcctcctaagctgctcatttacctg
gtgtctaacctggaatccggggtccctgaccgattcagtggcagcgggtctgggacagatttcactctcaccatcagcagc
ctgcaggctgaagatgtggcagtttattactgtcaacacattcgggaactgaccaggagtgaattcggcggagggaccaag
gtggagatcaaacgaactggtcagcccaaggctgccccctcggtcactctgttcccgccctcctctgaggagcttcaagcc
aacaaggccacactggtgtgtctcataagtgacttctacccgggagccgtgacagtggcctggaaggcagatagcagcccc
gtcaaggcgggagtggagaccaccacaccctccaaacaaagcaacaacaagtacgcggccagcagctatctgagcctgacg
cctgagcagtggaagtcccacagaagctacagctgccaggtcacgcatgaagggagcaccgtggagaagacagtggcccct
acagaatgttcatagtaa
(amino acids)
(SEQ ID NO: 580)
DIVMTQSPDSLAVSLGERATINCRASKSVSTSGYSYMHWYQQKPGQPPKLLIYLVSNLESGVPDRFSGSGSGTDFTLTISS
LQAEDVAVYYCQHIRELTRSEFGGGTKVEIKRTGQPKAAPSVTLFPPSSEELQANKATLVCLISDFYPGAVTVAWKADSSP
VKAGVETTTPSKQSNNKYAASSYLSLTPEQWKSHRSYSCQVTHEGSTVEKTVAPTECS**
Humanized C8 Kappa light chain sequence
(DNA)
(SEQ ID NO: 581)
gacatcgtgatgacccagtctccagactccctggctgtgtctctgggcgagagggccaccatcaactgcagggccagcaag
agtgttagcaccagcggctacagctacatgcactggtaccagcagaaaccaggacagcctcctaagctgctcatttacctg
gtgtctaacctggaatccggggtccctgaccgattcagtggcagcgggtctgggacagatttcactctcaccatcagcagc
ctgcaggctgaagatgtggcagtttattactgtcaacacattcgggaactgaccaggagtgaattcggcggagggaccaag
gtggagatcaaacgaactacggtggctgcaccatctgtcttcatcttcccgccatctgatgagcagttgaaatctggaact
gcctctgttgtgtgcctgctgaataacttctatcccagagaggccaaagtacagtggaaggtggataacgccctccaatcg
ggtaactcccaggagagtgtcacagagcaggacagcaaggacagcacctacagcctcagcagcaccctgacgctgagcaaa
gcagactacgagaaacacaaagtctacgcctgcgaagtcacccatcagggcctgagctcgcccgtcacaaagagcttcaac
aggggagagtgttagtaa
(amino acids)
(SEQ ID NO: 582)
DIVMTQSPDSLAVSLGERATINCRASKSVSTSGYSYMHWYQQKPGQPPKLLIYLVSNLESGVPDRFSGSGSGTDFTLTISS
LQAEDVAVYYCQHIRELTRSEFGGGTKVEIKRTTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPVTKSFNRGEC**
Humanized C8 Kappa light chain gBLOCk sequence:
(DNA)
(SEQ ID NO: 583)
agctggctaggtaagcttggtaccgagctcggatccacgccaccatggagacagacacactcctgctatgggtactgctgc
tctgggttccaggttccactggtgacgacatcgtgatgacccagtctccagactccctggctgtgtctctgggcgagaggg
ccaccatcaactgcagggccagcaagagtgttagcaccagcggctacagctacatgcactggtaccagcagaaaccaggac
agcctcctaagctgctcatttacctggtgtctaacctggaatccggggtccctgaccgattcagtggcagcgggtctggga
cagatttcactctcaccatcagcagcctgcaggctgaagatgtggcagtttattactgtcaacacattcgggaactgacca
ggagtgaattcggcggagggaccaaggtggagatcaaacgaactacggtggctgcaccatctgtcttcatcttcccgccat
ctgatgagcagttgaaatctggaactgcctctgttgtgtgcctgctgaataacttctatcccagagaggccaaagtacagt
ggaaggtggataacgccctccaatcgggtaactcccaggagagtgtcacagagcaggacagcaaggacagcacctacagcc
tcagcagcaccctgacgctgagcaaagcagactacgagaaacacaaagtctacgcctgcgaagtcacccatcagggcctga
gctcgcccgtcacaaagagcttcaacaggggagagtgttagtaagtttaaacccgctgatcagcctcgactgtgccttcta
gttgc
CAR-T E6 CD8 sequence:
(DNA)
(SEQ ID NO: 584)
gaggtccagctggttgagagtggcggtgggctggttaagcctggcggctccctgcggctgagctgcgccgcgagtggattt
actttcagccgatatgggatgagttgggtgcggcaagctcccgggaagaggctggaatgggtctcaacaatctccgggggg
ggcacttacatctattaccccgactcagtcaaggggagatttaccatttcacgagacaacgctaagaataccctgtatttg
cagatgaattctctgagagcagaggacacagctgtttactattgtacccgcgacaactatggcaggaactacgactacggt
atggactattggggacaagggacattggttacagtgagcagtggcggcgggggcagcggaggaggaggcagcggtgggggg
ggcagcgagatagtgctcacgcagtcacccgcgactctcagtctctcacctggggaacgagctaccctgacgtgctctgct
acctcctcagtgtcatatattcactggtatcagcaacggcccgggcagtcccctagattgctcatttatagtacctctaat
ctggcctcaggtatccctgcacgattttctggatctggttcaggttctgattacaccctcactatctctagcctggagcct
gaagactttgccgtttattactgccagcagaggtctagctccccattcacctttgggagtgggaccaaggttgaaattaaa
acgacaaccccggcccccagaccaccaacgccagcccccaccatcgccagccaacccctgtctctgagaccagaagcctgt
aggcctgccgccggtggagctgtgcacacaagaggactggatttcgcctgtgatatctacatttgggccccgctcgcaggc
acatgtggagtgctcctcctctccctggtgattaccctgtactgctgataa
(amino acids)
(SEQ ID NO: 585)
EVQLVESGGGLVKPGGSLRLSCAASGFTFSRYGMSWVRQAPGKRLEWVSTISGGGTYIYYPDSVKGRFTISRDNAKNTLY
LQMNSLRAEDTAVYYCTRDNYGRNYDYGMDYWGQGTLVTVSSGGGGSGGGGSGGGGSEIVLTQSPATLSLSPGERATLTC
SATSSVSYIHWYQQRPGQSPRLLIYSTSNLASGIPARFSGSGSGSDYTLTISSLEPEDFAVYYCQQRSSSPFTFGSGTKV
EIKTTTPAPRPPTPAPTIASQPLSLRPEACRPAAGGAVHTRGLDFACDIYIWAPLAGTCGVLLLSLVITLYC**
CAR-T C2 CD8 CD8 sequence:
N-CD8ls-huMNC2scFv-CD8ecd fragment- CD8 transmembrane-C
(DNA)
(SEQ ID NO: 586)
gaagtgcagctcgtagagagtggcgggggactggtgaagcccggtggaagcctcagactcagttgcgccgcctcaggtttc
actttttcaggttacgccatgtcctgggtaagacaggcaccggggaaaggactcgagtgggtgtctactatcagctcagga
ggcacttatatatattatcctgactctgtaaaaggccgatttacgatttctcgcgacaatgcaaagaactccctctacctc
caaatgaacagtcttagggcagaagacactgctgtatactattgtgcacgcctcggcggcgacaactactacgagtacttt
gacgtgtgggggaaagggactaccgtgacagtttcaagcggaggaggtggctcaggtggaggcgggtcaggggggggagga
agtgatattgtgctcacacaatccccagcctccctggctgtgtctcccggccaacgcgctacaattacatgtcgggcctcc
aaaagcgtgagcaccagcggctacagctacatgcactggtatcaacagaaaccaggacaaccccccaaactgttgatttat
ctcgcttcaaacttggagtccggcgtgcctgcgcgcttttcagggagtgggagcggcacagattttacgctgactatcaac
cccgtagaagcaaacgatacagcgaattattattgtcaacattcccgggaactcccctttacgttcggcgggggcacaaag
gtcgaaattaagagaaccacgacaaccccggcccccagaccaccaacgccagcccccaccatcgccagccaacccctgtct
ctgagaccagaagcctgtaggcctgccgccggtggagctgtgcacacaagaggactggatttcgcctgtgatatctacatt
tgggccccgctcgcaggcacatgtggagtgctcctcctctccctggtgattaccctgtactgctgataa
(amino acids)
(SEQ ID NO: 587)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRTTTTPAPRPPTPAPTIASQPLSLRPEACRPAAGGAVHTRGLDFACDIYIWAPLAGTCGVLLLSLVITLYC**
CD8/4-1BB sequence
N- CD8 transmembrane- 4-1BB-C
(DNA)
(SEQ ID NO: 588)
acgacaaccccggcccccagaccaccaacgccagcccccaccatcgccagccaacccctgtctctgagaccagaagcctgt
aggcctgccgccggtggagctgtgcacacaagaggactggatttcgcctgtgatatctacatttgggccccgctcgcaggc
acatgtggagtgctcctcctctccctggtgattaccctgtactgcaaaaggggccgcaaaaaactcctttacatttttaag
cagccttttatgaggccagtacagacgactcaagaggaagacgggtgctcatgccgctttcctgaggaggaggaaggaggg
tgcgaactgtgataa
(amino acids)
(SEQ ID NO: 589)
TTTPAPRPPTPAPTIASQPLSLRPEACRPAAGGAVHTRGLDFACDIYIWAPLAGTCGVLLLSLVITLYCKRGRKKLLYIF
KQPFMRPVQTTQEEDGCSCRFPEEEEGGCEL**
CD8/CD28 sequence
N- CD8 transmembrane- CD28-C
(DNA)
(SEQ ID NO: 590)
acgacaaccccggcccccagaccaccaacgccagcccccaccatcgccagccaacccctgtctctgagaccagaagcctgt
aggcctgccgccggtggagctgtgcacacaagaggactggatttcgcctgtgatatctacatttgggccccgctcgcaggc
acatgtggagtgctcctcctctccctggtgattaccctgtactgcagaagcaagcggtctcggctcctgcattctgattac
atgaacatgaccccaagaagaccaggccccaccaggaaacattaccagccctacgctccgccacgcgacttcgctgcctac
cggtcctgataa
(amino acids)
(SEQ ID NO: 591)
TTTPAPRPPTPAPTIASQPLSLRPEACRPAAGGAVHTRGLDFACDIYIWAPLAGTCGVLLLSLVITLYCRSKRSRLLHSD
YMNMTPRRPGPTRKHYQPYAPPRDFAAYRS**
CD8/CD3z sequence:
N- CD8 transmembrane- CD3zeta-C
(DNA)
(SEQ ID NO: 592)
acgacaaccccggcccccagaccaccaacgccagcccccaccatcgccagccaacccctgtctctgagaccagaagcctgt
aggcctgccgccggtggagctgtgcacacaagaggactggatttcgcctgtgatatctacatttgggccccgctcgcaggc
acatgtggagtgctcctcctctccctggtgattaccctgtactgccgcgttaagttctcccgatcagccgacgcgcctgct
tacaagcagggccagaaccaactgtacaacgagctgaatctcggtagacgggaagagtacgacgtgttggacaaacggaga
ggccgcgacccagaaatgggcggcaagcctcgcaggaaaaacccccaggagggactgtacaatgagttgcagaaagataag
atggcagaagcttatagcgagatcggaatgaagggggaaaggagacgagggaaaggacacgacggcctttatcagggcctg
tccacagcaacaaaagatacgtatgacgccctccatatgcaggcacttccaccacggtgataa
(amino acids)
(SEQ ID NO: 593)
TTTPAPRPPTPAPTIASQPLSLRPEACRPAAGGAVHTRGLDFACDIYIWAPLAGTCGVLLLSLVITLYCRVKFSRSADAP
AYKQGQNQLYNELNLGRREEYDVLDKRRGRDPEMGGKPRRKNPQEGLYNELQKDKMAEAYSEIGMKGERRRGKGHDGLYQ
GLSTATKDTYDALHMQALPPR**
CD8/CD28/CD3z sequence:
N- CD8 transmembrane- CD28- CD3zeta-C
(DNA)
(SEQ ID NO: 594)
acgacaaccccggcccccagaccaccaacgccagcccccaccatcgccagccaacccctgtctctgagaccagaagcctgt
aggcctgccgccggtggagctgtgcacacaagaggactggatttcgcctgtgatatctacatttgggccccgctcgcaggc
acatgtggagtgctcctcctctccctggtgattaccctgtactgcagaagcaagcggtctcggctcctgcattctgattac
atgaacatgaccccaagaagaccaggccccaccaggaaacattaccagccctacgctccgccacgcgacttcgctgcctac
cggtcccgcgttaagttctcccgatcagccgacgcgcctgcttacaagcagggccagaaccaactgtacaacgagctgaat
ctcggtagacgggaagagtacgacgtgttggacaaacggagaggccgcgacccagaaatgggcggcaagcctcgcaggaaa
aacccccaggagggactgtacaatgagttgcagaaagataagatggcagaagcttatagcgagatcggaatgaagggggaa
aggagacgagggaaaggacacgacggcctttatcagggcctgtccacagcaacaaaagatacgtatgacgccctccatatg
caggcacttccaccacggtgataa
(amino acids)
(SEQ ID NO: 595)
TTTPAPRPPTPAPTIASQPLSLRPEACRPAAGGAVHTRGLDFACDIYIWAPLAGTCGVLLLSLVITLYCRSKRSRLLHSD
YMNMTPRRPGPTRKHYQPYAPPRDFAAYRSRVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDTYDALHMQALPPR**
CD8/4-1BB/CD3z sequence:
N- CD8 transmembrane- 4-1BB- CD3zeta-C
(DNA)
(SEQ ID NO: 596)
acgacaaccccggcccccagaccaccaacgccagcccccaccatcgccagccaacccctgtctctgagaccagaagcctgt
aggcctgccgccggtggagctgtgcacacaagaggactggatttcgcctgtgatatctacatttgggccccgctcgcaggc
acatgtggagtgctcctcctctccctggtgattaccctgtactgcaaaaggggccgcaaaaaactcctttacatttttaag
cagccttttatgaggccagtacagacgactcaagaggaagacgggtgctcatgccgctttcctgaggaggaggaaggaggg
tgcgaactgcgcgttaagttctcccgatcagccgacgcgcctgcttacaagcagggccagaaccaactgtacaacgagctg
aatctcggtagacgggaagagtacgacgtgttggacaaacggagaggccgcgacccagaaatgggcggcaagcctcgcagg
aaaaacccccaggagggactgtacaatgagttgcagaaagataagatggcagaagcttatagcgagatcggaatgaagggg
gaaaggagacgagggaaaggacacgacggcctttatcagggcctgtccacagcaacaaaagatacgtatgacgccctccat
atgcaggcacttccaccacggtgataa
(amino acids)
(SEQ ID NO: 597)
TTTPAPRPPTPAPTIASQPLSLRPEACRPAAGGAVHTRGLDFACDIYIWAPLAGTCGVLLLSLVITLYCKRGRKKLLYIF
KQPFMRPVQTTQEEDGCSCRFPEEEEGGCELRVKFSRSADAPAYKQGQNQLYNELNLGRREEYDVLDKRRGRDPEMGGKP
RRKNPQEGLYNELQKDKMAEAYSEIGMKGERRRGKGHDGLYQGLSTATKDTYDALHMQALPPR**
CD8/CD28/4-1BB/CD3z sequence:
N- CD8 transmembrane- CD28- 4-1BB- CD3zeta-C
(DNA)
(SEQ ID NO: 598)
acgacaaccccggcccccagaccaccaacgccagcccccaccatcgccagccaacccctgtctctgagaccagaagcctgt
aggcctgccgccggtggagctgtgcacacaagaggactggatttcgcctgtgatatctacatttgggccccgctcgcaggc
acatgtggagtgctcctcctctccctggtgattaccctgtactgcagaagcaagcggtctcggctcctgcattctgattac
atgaacatgaccccaagaagaccaggccccaccaggaaacattaccagccctacgctccgccacgcgacttcgctgcctac
cggtccaaaaggggccgcaaaaaactcctttacatttttaagcagccttttatgaggccagtacagacgactcaagaggaa
gacgggtgctcatgccgctttcctgaggaggaggaaggagggtgcgaactgcgcgttaagttctcccgatcagccgacgcg
cctgcttacaagcagggccagaaccaactgtacaacgagctgaatctcggtagacgggaagagtacgacgtgttggacaaa
cggagaggccgcgacccagaaatgggcggcaagcctcgcaggaaaaacccccaggagggactgtacaatgagttgcagaaa
gataagatggcagaagcttatagcgagatcggaatgaagggggaaaggagacgagggaaaggacacgacggcctttatcag
ggcctgtccacagcaacaaaagatacgtatgacgccctccatatgcaggcacttccaccacggtgataa
(amino acids)
(SEQ ID NO: 599)
TTTPAPRPPTPAPTIASQPLSLRPEACRPAAGGAVHTRGLDFACDIYIWAPLAGTCGVLLLSLVITLYCRSKRSRLLHSD
YMNMTPRRPGPTRKHYQPYAPPRDFAAYRSKRGRKKLLYIFKQPFMRPVQTTQEEDGCSCRFPEEEEGGCELRVKFSRSA
DAPAYKQGQNQLYNELNLGRREEYDVLDKRRGRDPEMGGKPRRKNPQEGLYNELQKDKMAEAYSEIGMKGERRRGKGHDG
LYQGLSTATKDTYDALHMQALPPR**
CAR-T C3 CD8/CD8/4-1BB/CD3z sequence:
N-CD8ls-huMNC3scFv-CD8ecd fragment- CD8 transmembrane- 4-1BB- CD3zeta-C
(DNA)
(SEQ ID NO: 600)
atggccctgcccgtgaccgctttgctgctccccctggcgctgctgctgcacgccgccaggccacaggttcagctggtgcag
tctggagctgaggtgaagaagcctggggcctcagtgaaggtctcctgcaaggcttctggttacacctttaccgactacgcc
atgaactgggtgcgacaggcccctggacaagggcttgagtggatgggagtgatcagcaccttcagcggtaacacaaacttc
aaccagaagttcaagggcagagtcaccatgaccacagacacatccacgagcacagcctacatggagctgaggagcctgaga
tctgacgacacggccgtgtattactgtgcgagaagcgactactacggcccatacttcgactactggggccagggcaccacc
ctgaccgtgtccagcggcggtggcggatccggcggtggcggatccggcggtggcggatccgatattgtgatgacccagact
ccactctctctgtccgtcacccctggacagccggcctccatctcctgcaggtctagtcagaccattgtccatagtaatgga
aacacctatttggagtggtacctgcagaagccaggccagtctccacagctcctgatctataaggtttccaaccggttctct
ggagtgccagataggttcagtggcagcgggtcagggacagatttcacactgaaaatcagccgggtggaggctgaggatgtt
ggggtttattactgcttccaaggtagccacgtgcctttcaccttcggcggagggaccaaggtggagatcaaacgaactacg
acaaccccggcccccagaccaccaacgccagcccccaccatcgccagccaacccctgtctctgagaccagaagcctgtagg
cctgccgccggtggagctgtgcacacaagaggactggatttcgcctgtgatatctacatttgggccccgctcgcaggcaca
tgtggagtgctcctcctctccctggtgattaccctgtactgcaaaaggggccgcaaaaaactcctttacatttttaagcag
ccttttatgaggccagtacagacgactcaagaggaagacgggtgctcatgccgctttcctgaggaggaggaaggagggtgc
gaactgcgcgttaagttctcccgatcagccgacgcgcctgcttacaagcagggccagaaccaactgtacaacgagctgaat
ctcggtagacgggaagagtacgacgtgttggacaaacggagaggccgcgacccagaaatgggcggcaagcctcgcaggaaa
aacccccaggagggactgtacaatgagttgcagaaagataagatggcagaagcttatagcgagatcggaatgaagggggaa
aggagacgagggaaaggacacgacggcctttatcagggcctgtccacagcaacaaaagatacgtatgacgccctccatatg
caggcacttccaccacggtgataa
(amino acids)
(SEQ ID NO: 601)
QVQLVQSGAEVKKPGASVKVSCKASGYTFTDYAMNWVRQAPGQGLEWMGVISTFSGNTNFNQKFKGRVTMTTDTSTSTAY
MELRSLRSDDTAVYYCARSDYYGPYFDYWGQGTTLTVSSGGGGSGGGGSGGGGSDIVMTQTPLSLSVTPGQPASISCRSS
QTIVHSNGNTYLEWYLQKPGQSPQLLIYKVSNRFSGVPDRFSGSGSGTDFTLKISRVEAEDVGVYYCFQGSHVPFTFGGG
TKVEIKRTTTTPAPRPPTPAPTIASQPLSLRPEACRPAAGGAVHTRGLDFACDIYIWAPLAGTCGVLLLSLVITLYCKRG
RKKLLYIFKQPFMRPVQTTQEEDGCSCRFPEEEEGGCELRVKFSRSADAPAYKQGQNQLYNELNLGRREEYDVLDKRRGR
DPEMGGKPRRKNPQEGLYNELQKDKMAEAYSEIGMKGERRRGKGHDGLYQGLSTATKDTYDALHMQALPPR**
C3 CAR gBLOCK 1 sequence:
(DNA)
(SEQ ID NO: 602)
atccacgctgttttgacctccatagaagattctagagctagctgtagagcttggtaccgagggccaccatggccctgcccg
tgaccgctttgctgctccccctggcgctgctgctgcacgccgccaggccacaggttcagctggtgcagtctggagctgagg
tgaagaagcctggggcctcagtgaaggtctcctgcaaggcttctggttacacctttaccgactacgccatgaactgggtgc
gacaggcccctggacaagggcttgagtggatgggagtgatcagcaccttcagcggtaacacaaacttcaaccagaagttca
agggcagagtcaccatgaccacagacacatccacgagcacagcctacatggagctgaggagcctgagatctgacgacacgg
ccgtgtattactgtgcgagaagcgactactacggcccatacttcgactactggggccagggcaccaccctgaccgtgtcca
gcggcggtggcggatccggcggtggcggatccggcggtggcggatccgatattgtgatgacccagactccactctctctgt
C3 CAR gBLOCK 2 sequence:
(DNA)
(SEQ ID NO: 603)
tattgtgatgacccagactccactctctctgtccgtcacccctggacagccggcctccatctcctgcaggtctagtcagac
cattgtccatagtaatggaaacacctatttggagtggtacctgcagaagccaggccagtctccacagctcctgatctataa
ggtttccaaccggttctctggagtgccagataggttcagtggcagcgggtcagggacagatttcacactgaaaatcagccg
ggtggaggctgaggatgttggggtttattactgcttccaaggtagccacgtgcctttcaccttcggcggagggaccaaggt
ggagatcaaacgaactacgacaaccccggcccccagaccaccaacgccagcccccaccatcgccagccaacccctgtctct
gagaccagaagcctgtaggcctgccgccggtggagctgtgcacacaagaggactggatttcgcctgtgatatctacatttg
ggccccgctcgcaggcacatgtg
E6 scFV gBLOCK 1 sequence:
(DNA)
(SEQ ID NO: 604)
tgctctgggttccaggttccactggtgacgcggcccagccggccgaggtgcagctggtggagtctgggggaggcctggtca
agcctggggggtccctgagactctcctgtgcagcctctggattcaccttcagtaggtatggcatgagctgggtccgccagg
ctccagggaagaggctggagtgggtctcaaccattagtggcggaggcacctacatatactacccagactcagtgaagggcc
gattcaccatctccagagacaacgccaagaacaccctgtatctgcaaatgaacagcctgagagccgaggacacggctgtgt
attactgtaccagagataactatggccgcaactatgattatggcatggattattggggccagggcaccctggtgaccgtga
gcagcggcggtggcggatccggcggtggcggatccggcggtggcggatcc
E6 scFV gBLOCK 2 sequence:
(DNA)
(SEQ ID NO: 605)
ggcggtggcggatccggcggtggcggatccggcggtggcggatccgaaattgtgttgacacagtctccagccaccctgtct
ttgtctccaggggaaagagccaccctcacctgcagcgccaccagcagtgttagctacatccactggtaccaacagaggcct
ggccagagccccaggctcctcatctatagcacctccaacctggccagcggcatcccagccaggttcagtggcagtgggtct
gggagcgactacactctcaccatcagcagcctagagcctgaagattttgcagtttattactgtcagcagcgtagcagctcc
cctttcacctttggcagcggcaccaaagtggaaattaaaaccggtcatcatcaccatcaccactgataagtttaaacccgc
tgatcagcctcgactgtgccttctagt
CAR-T C2 CD8/CD8/CD3z sequence:
N-CD8ls-huMNC2scFv-CD8ecd fragment- CD8 transmembrane- CD3zeta-C
(DNA)
(SEQ ID NO: 606)
atggccttgccagtgacggccctgctgctgccattggctcttctgttgcacgctgccaggcctgaagtgcagctcgtagag
agtggcgggggactggtgaagcccggtggaagcctcagactcagttgcgccgcctcaggtttcactttttcaggttacgcc
atgtcctgggtaagacaggcaccggggaaaggactcgagtgggtgtctactatcagctcaggaggcacttatatatattat
cctgactctgtaaaaggccgatttacgatttctcgcgacaatgcaaagaactccctctacctccaaatgaacagtcttagg
gcagaagacactgctgtatactattgtgcacgcctcggcggcgacaactactacgagtactttgacgtgtgggggaaaggg
actaccgtgacagtttcaagcggaggaggtggctcaggtggaggcgggtcaggggggggaggaagtgatattgtgctcaca
caatccccagcctccctggctgtgtctcccggccaacgcgctacaattacatgtcgggcctccaaaagcgtgagcaccagc
ggctacagctacatgcactggtatcaacagaaaccaggacaaccccccaaactgttgatttatctcgcttcaaacttggag
tccggcgtgcctgcgcgcttttcagggagtgggagcggcacagattttacgctgactatcaaccccgtagaagcaaacgat
acagcgaattattattgtcaacattcccgggaactcccctttacgttcggcgggggcacaaaggtcgaaattaagagaacc
acgacaaccccggcccccagaccaccaacgccagcccccaccatcgccagccaacccctgtctctgagaccagaagcctgt
aggcctgccgccggtggagctgtgcacacaagaggactggatttcgcctgtgatatctacatttgggccccgctcgcaggc
acatgtggagtgctcctcctctccctggtgattaccctgtactgccgcgttaagttctcccgatcagccgacgcgcctgct
tacaagcagggccagaaccaactgtacaacgagctgaatctcggtagacgggaagagtacgacgtgttggacaaacggaga
ggccgcgacccagaaatgggcggcaagcctcgcaggaaaaacccccaggagggactgtacaatgagttgcagaaagataag
atggcagaagcttatagcgagatcggaatgaagggggaaaggagacgagggaaaggacacgacggcctttatcagggcctg
tccacagcaacaaaagatacgtatgacgccctccatatgcaggcacttccaccacggtgataa
(amino acids)
(SEQ ID NO: 607)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTTTTPAPRPPTPAPTIASQPLSLRPEACRPAAGGAVHTRGLDFACDIYIWA
PLAGTCGVLLLSLVITLYCRVKFSRSADAPAYKQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPR**
CAR-T C2 CD8/CD8/CD28/CD3z sequence:
N-CD8ls-huMNC2scFv-CD8ecd fragment- CD8 transmembrane- CD28- CD3zeta-C
(DNA)
(SEQ ID NO: 608)
atggccttgccagtgacggccctgctgctgccattggctcttctgttgcacgctgccaggcctgaagtgcagctcgtagag
agtggcgggggactggtgaagcccggtggaagcctcagactcagttgcgccgcctcaggtttcactttttcaggttacgcc
atgtcctgggtaagacaggcaccggggaaaggactcgagtgggtgtctactatcagctcaggaggcacttatatatattat
cctgactctgtaaaaggccgatttacgatttctcgcgacaatgcaaagaactccctctacctccaaatgaacagtcttagg
gcagaagacactgctgtatactattgtgcacgcctcggcggcgacaactactacgagtactttgacgtgtgggggaaaggg
actaccgtgacagtttcaagcggaggaggtggctcaggtggaggcgggtcaggggggggaggaagtgatattgtgctcaca
caatccccagcctccctggctgtgtctcccggccaacgcgctacaattacatgtcgggcctccaaaagcgtgagcaccagc
ggctacagctacatgcactggtatcaacagaaaccaggacaaccccccaaactgttgatttatctcgcttcaaacttggag
tccggcgtgcctgcgcgcttttcagggagtgggagcggcacagattttacgctgactatcaaccccgtagaagcaaacgat
acagcgaattattattgtcaacattcccgggaactcccctttacgttcggcgggggcacaaaggtcgaaattaagagaacc
acgacaaccccggcccccagaccaccaacgccagcccccaccatcgccagccaacccctgtctctgagaccagaagcctgt
aggcctgccgccggtggagctgtgcacacaagaggactggatttcgcctgtgatatctacatttgggccccgctcgcaggc
acatgtggagtgctcctcctctccctggtgattaccctgtactgcagaagcaagcggtctcggctcctgcattctgattac
atgaacatgaccccaagaagaccaggccccaccaggaaacattaccagccctacgctccgccacgcgacttcgctgcctac
cggtcccgcgttaagttctcccgatcagccgacgcgcctgcttacaagcagggccagaaccaactgtacaacgagctgaat
ctcggtagacgggaagagtacgacgtgttggacaaacggagaggccgcgacccagaaatgggcggcaagcctcgcaggaaa
aacccccaggagggactgtacaatgagttgcagaaagataagatggcagaagcttatagcgagatcggaatgaagggggaa
aggagacgagggaaaggacacgacggcctttatcagggcctgtccacagcaacaaaagatacgtatgacgccctccatatg
caggcacttccaccacggtgataa
(amino acids)
(SEQ ID NO: 609)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTTTTPAPRPPTPAPTIASQPLSLRPEACRPAAGGAVHTRGLDFACDIYIWA
PLAGTCGVLLLSLVITLYCRSKRSRLLHSDYMNMTPRRPGPTRKHYQPYAPPRDFAAYRSRVKFSRSADAPAYKQGQNQL
YNELNLGRREEYDVLDKRRGRDPEMGGKPRRKNPQEGLYNELQKDKMAEAYSEIGMKGERRRGKGHDGLYQGLSTATKDT
YDALHMQALPPR**
CAR-T C2 CD8/CD8/4-1BB/CD3z sequence #13:
N-CD8ls-huMNC2scFv-CD8ecd fragment- CD8 transmembrane- 4-1BB- CD3zeta-C
(DNA)
(SEQ ID NO: 610)
atggccttgccagtgacggccctgctgctgccattggctcttctgttgcacgctgccaggcctgaagtgcagctcgtagag
agtggcgggggactggtgaagcccggtggaagcctcagactcagttgcgccgcctcaggtttcactttttcaggttacgcc
atgtcctgggtaagacaggcaccggggaaaggactcgagtgggtgtctactatcagctcaggaggcacttatatatattat
cctgactctgtaaaaggccgatttacgatttctcgcgacaatgcaaagaactccctctacctccaaatgaacagtcttagg
gcagaagacactgctgtatactattgtgcacgcctcggcggcgacaactactacgagtactttgacgtgtgggggaaaggg
actaccgtgacagtttcaagcggaggaggtggctcaggtggaggcgggtcaggggggggaggaagtgatattgtgctcaca
caatccccagcctccctggctgtgtctcccggccaacgcgctacaattacatgtcgggcctccaaaagcgtgagcaccagc
ggctacagctacatgcactggtatcaacagaaaccaggacaaccccccaaactgttgatttatctcgcttcaaacttggag
tccggcgtgcctgcgcgcttttcagggagtgggagcggcacagattttacgctgactatcaaccccgtagaagcaaacgat
acagcgaattattattgtcaacattcccgggaactcccctttacgttcggcgggggcacaaaggtcgaaattaagagaacc
acgacaaccccggcccccagaccaccaacgccagcccccaccatcgccagccaacccctgtctctgagaccagaagcctgt
aggcctgccgccggtggagctgtgcacacaagaggactggatttcgcctgtgatatctacatttgggccccgctcgcaggc
acatgtggagtgctcctcctctccctggtgattaccctgtactgcaaaaggggccgcaaaaaactcctttacatttttaag
cagccttttatgaggccagtacagacgactcaagaggaagacgggtgctcatgccgctttcctgaggaggaggaaggaggg
tgcgaactgcgcgttaagttctcccgatcagccgacgcgcctgcttacaagcagggccagaaccaactgtacaacgagctg
aatctcggtagacgggaagagtacgacgtgttggacaaacggagaggccgcgacccagaaatgggcggcaagcctcgcagg
aaaaacccccaggagggactgtacaatgagttgcagaaagataagatggcagaagcttatagcgagatcggaatgaagggg
gaaaggagacgagggaaaggacacgacggcctttatcagggcctgtccacagcaacaaaagatacgtatgacgccctccat
atgcaggcacttccaccacggtgataa
(amino acids)
(SEQ ID NO: 611)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTTTTPAPRPPTPAPTIASQPLSLRPEACRPAAGGAVHTRGLDFACDIYIWA
PLAGTCGVLLLSLVITLYCKRGRKKLLYIFKQPFMRPVQTTQEEDGCSCRFPEEEEGGCELRVKFSRSADAPAYKQGQNQ
LYNELNLGRREEYDVLDKRRGRDPEMGGKPRRKNPQEGLYNELQKDKMAEAYSEIGMKGERRRGKGHDGLYQGLSTATKD
TYDALHMQALPPR**
CAR-T C2 CD8/CD8/OX40/CD3z sequence:
N-CD8ls-huMNC2scFv-CD8ecd fragment- CD8 transmembrane- OX40- CD3zeta-C
(DNA)
(SEQ ID NO: 612)
atggccttgccagtgacggccctgctgctgccattggctcttctgttgcacgctgccaggcctgaagtgcagctcgtagag
agtggcgggggactggtgaagcccggtggaagcctcagactcagttgcgccgcctcaggtttcactttttcaggttacgcc
atgtcctgggtaagacaggcaccggggaaaggactcgagtgggtgtctactatcagctcaggaggcacttatatatattat
cctgactctgtaaaaggccgatttacgatttctcgcgacaatgcaaagaactccctctacctccaaatgaacagtcttagg
gcagaagacactgctgtatactattgtgcacgcctcggcggcgacaactactacgagtactttgacgtgtgggggaaaggg
actaccgtgacagtttcaagcggaggaggtggctcaggtggaggcgggtcaggggggggaggaagtgatattgtgctcaca
caatccccagcctccctggctgtgtctcccggccaacgcgctacaattacatgtcgggcctccaaaagcgtgagcaccagc
ggctacagctacatgcactggtatcaacagaaaccaggacaaccccccaaactgttgatttatctcgcttcaaacttggag
tccggcgtgcctgcgcgcttttcagggagtgggagcggcacagattttacgctgactatcaaccccgtagaagcaaacgat
acagcgaattattattgtcaacattcccgggaactcccctttacgttcggcgggggcacaaaggtcgaaattaagagaacc
acgacaaccccggcccccagaccaccaacgccagcccccaccatcgccagccaacccctgtctctgagaccagaagcctgt
aggcctgccgccggtggagctgtgcacacaagaggactggatttcgcctgtgatatctacatttgggccccgctcgcaggc
acatgtggagtgctcctcctctccctggtgattaccctgtactgccggagggaccagaggctgccccccgatgcccacaag
ccccctgggggaggcagtttccggacccccatccaagaggagcaggccgacgcccactccaccctggccaagatccgcgtt
aagttctcccgatcagccgacgcgcctgcttacaagcagggccagaaccaactgtacaacgagctgaatctcggtagacgg
gaagagtacgacgtgttggacaaacggagaggccgcgacccagaaatgggcggcaagcctcgcaggaaaaacccccaggag
ggactgtacaatgagttgcagaaagataagatggcagaagcttatagcgagatcggaatgaagggggaaaggagacgaggg
aaaggacacgacggcctttatcagggcctgtccacagcaacaaaagatacgtatgacgccctccatatgcaggcacttcca
ccacggtgataa
(amino acids)
(SEQ ID NO: 613)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTTTTPAPRPPTPAPTIASQPLSLRPEACRPAAGGAVHTRGLDFACDIYIWA
PLAGTCGVLLLSLVITLYCRRDQRLPPDAHKPPGGGSFRTPIQEEQADAHSTLAKIRVKFSRSADAPAYKQGQNQLYNEL
NLGRREEYDVLDKRRGRDPEMGGKPRRKNPQEGLYNELQKDKMAEAYSEIGMKGERRRGKGHDGLYQGLSTATKDTYDAL
HMQALPPR**
CAR-T C2 CD8/CD8/CD28/OX40/CD3z sequence:
N-CD8ls-huMNC2scFv-CD8ecd fragment- CD8 transmembrane- CD28- OX40- CD3zeta-C
(DNA)
(SEQ ID NO: 614)
atggccttgccagtgacggccctgctgctgccattggctcttctgttgcacgctgccaggcctgaagtgcagctcgtagag
agtggcgggggactggtgaagcccggtggaagcctcagactcagttgcgccgcctcaggtttcactttttcaggttacgcc
atgtcctgggtaagacaggcaccggggaaaggactcgagtgggtgtctactatcagctcaggaggcacttatatatattat
cctgactctgtaaaaggccgatttacgatttctcgcgacaatgcaaagaactccctctacctccaaatgaacagtcttagg
gcagaagacactgctgtatactattgtgcacgcctcggcggcgacaactactacgagtactttgacgtgtgggggaaaggg
actaccgtgacagtttcaagcggaggaggtggctcaggtggaggcgggtcaggggggggaggaagtgatattgtgctcaca
caatccccagcctccctggctgtgtctcccggccaacgcgctacaattacatgtcgggcctccaaaagcgtgagcaccagc
ggctacagctacatgcactggtatcaacagaaaccaggacaaccccccaaactgttgatttatctcgcttcaaacttggag
tccggcgtgcctgcgcgcttttcagggagtgggagcggcacagattttacgctgactatcaaccccgtagaagcaaacgat
acagcgaattattattgtcaacattcccgggaactcccctttacgttcggcgggggcacaaaggtcgaaattaagagaacc
acgacaaccccggcccccagaccaccaacgccagcccccaccatcgccagccaacccctgtctctgagaccagaagcctgt
aggcctgccgccggtggagctgtgcacacaagaggactggatttcgcctgtgatatctacatttgggccccgctcgcaggc
acatgtggagtgctcctcctctccctggtgattaccctgtactgcagaagcaagcggtctcggctcctgcattctgattac
atgaacatgaccccaagaagaccaggccccaccaggaaacattaccagccctacgctccgccacgcgacttcgctgcctac
cggtcccggagggaccagaggctgccccccgatgcccacaagccccctgggggaggcagtttccggacccccatccaagag
gagcaggccgacgcccactccaccctggccaagatccgcgttaagttctcccgatcagccgacgcgcctgcttacaagcag
ggccagaaccaactgtacaacgagctgaatctcggtagacgggaagagtacgacgtgttggacaaacggagaggccgcgac
ccagaaatgggcggcaagcctcgcaggaaaaacccccaggagggactgtacaatgagttgcagaaagataagatggcagaa
gcttatagcgagatcggaatgaagggggaaaggagacgagggaaaggacacgacggcctttatcagggcctgtccacagca
acaaaagatacgtatgacgccctccatatgcaggcacttccaccacggtgataa
(amino acids)
(SEQ ID NO: 615)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTTTTPAPRPPTPAPTIASQPLSLRPEACRPAAGGAVHTRGLDFACDIYIWA
PLAGTCGVLLLSLVITLYCRSKRSRLLHSDYMNMTPRRPGPTRKHYQPYAPPRDFAAYRSRRDQRLPPDAHKPPGGGSFR
TPIQEEQADAHSTLAKIRVKFSRSADAPAYKQGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPR**
CAR-T E6 CD8/CD8/OX40/CD3z sequence:
N-CD8ls-huMNE6scFv-CD8ecd fragment- CD8 transmembrane- OX40- CD3zeta-C
(DNA)
(SEQ ID NO: 616)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggggggggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaaacgacaaccccggccccc
agaccaccaacgccagcccccaccatcgccagccaacccctgtctctgagaccagaagcctgtaggcctgccgccggtgga
gctgtgcacacaagaggactggatttcgcctgtgatatctacatttgggccccgctcgcaggcacatgtggagtgctcctc
ctctccctggtgattaccctgtactgccggagggaccagaggctgccccccgatgcccacaagccccctgggggaggcagt
ttccggacccccatccaagaggagcaggccgacgcccactccaccctggccaagatccgcgttaagttctcccgatcagcc
gacgcgcctgcttacaagcagggccagaaccaactgtacaacgagctgaatctcggtagacgggaagagtacgacgtgttg
gacaaacggagaggccgcgacccagaaatgggcggcaagcctcgcaggaaaaacccccaggagggactgtacaatgagttg
cagaaagataagatggcagaagcttatagcgagatcggaatgaagggggaaaggagacgagggaaaggacacgacggcctt
tatcagggcctgtccacagcaacaaaagatacgtatgacgccctccatatgcaggcacttccaccacggtgataa
(amino acids)
(SEQ ID NO: 617)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKTTTPAPRPPTPAPTIASQPLSLRPEACRPAAGGAVHTRGLDFACDIYIWAPLAGTC
GVLLLSLVITLYCRRDQRLPPDAHKPPGGGSFRTPIQEEQADAHSTLAKIRVKFSRSADAPAYKQGQNQLYNELNLGRRE
EYDVLDKRRGRDPEMGGKPRRKNPQEGLYNELQKDKMAEAYSEIGMKGERRRGKGHDGLYQGLSTATKDTYDALHMQALP
PR**
CAR-T E6 CD8/CD8/CD28/OX40/CD3z sequence:
N-CD8ls-huMNC2scFv-CD8ecd fragment- CD8 transmembrane- CD28- OX40- CD3zeta-C
(DNA)
(SEQ ID NO: 618)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggggggggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaaacgacaaccccggccccc
agaccaccaacgccagcccccaccatcgccagccaacccctgtctctgagaccagaagcctgtaggcctgccgccggtgga
gctgtgcacacaagaggactggatttcgcctgtgatatctacatttgggccccgctcgcaggcacatgtggagtgctcctc
ctctccctggtgattaccctgtactgcagaagcaagcggtctcggctcctgcattctgattacatgaacatgaccccaaga
agaccaggccccaccaggaaacattaccagccctacgctccgccacgcgacttcgctgcctaccggtcccggagggaccag
aggctgccccccgatgcccacaagccccctgggggaggcagtttccggacccccatccaagaggagcaggccgacgcccac
tccaccctggccaagatccgcgttaagttctcccgatcagccgacgcgcctgcttacaagcagggccagaaccaactgtac
aacgagctgaatctcggtagacgggaagagtacgacgtgttggacaaacggagaggccgcgacccagaaatgggcggcaag
cctcgcaggaaaaacccccaggagggactgtacaatgagttgcagaaagataagatggcagaagcttatagcgagatcgga
atgaagggggaaaggagacgagggaaaggacacgacggcctttatcagggcctgtccacagcaacaaaagatacgtatgac
gccctccatatgcaggcacttccaccacggtgataa
(amino acids)
(SEQ ID NO: 619)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKTTTPAPRPPTPAPTIASQPLSLRPEACRPAAGGAVHTRGLDFACDIYIWAPLAGTC
GVLLLSLVITLYCRSKRSRLLHSDYMNMTPRRPGPTRKHYQPYAPPRDFAAYRSRRDQRLPPDAHKPPGGGSFRTPIQEE
QADAHSTLAKIRVKFSRSADAPAYKQGQNQLYNELNLGRREEYDVLDKRRGRDPEMGGKPRRKNPQEGLYNELQKDKMAE
AYSEIGMKGERRRGKGHDGLYQGLSTATKDTYDALHMQALPPR**
MUC1 truncated cytoplasmic sequence
(amino acids)
(SEQ ID NO: 620)
SNIKFRPGSVVVQLTLAFREGTINVHDVETQFNQYKTEAASRY
MUC1 truncated cytoplasmic sequence
(amino acids)
(SEQ ID NO: 621)
SVVVQLTLAFREGTINVHDVETQFNQYKTEAASRY
MUC1 truncated cytoplasmic sequence
(amino acids)
(SEQ ID NO: 622)
VQLTLAFREGTINVHDVETQFNQY
MUC1 truncated cytoplasmic sequence
(amino acids)
(SEQ ID NO: 623)
SNIKFRPGSVVVQLTLAFREGTIN
Primers
(SEQ ID NO: 624)
attctaagcttgggccaccatggaactg
(SEQ ID NO: 625)
tctagagtttaaacttactatttacccggagacagggagag
(SEQ ID NO: 626)
agtatggcccagccggccgaggtgcagctggtggagtctgg
(SEQ ID NO: 627)
tagaaggcacagtcgaggctgatcag
(SEQ ID NO: 628)
attctaagcttgggccaccatggaagc
(SEQ ID NO: 629)
tctagagtttaaacttactaacactctcccctgttgaagc
(SEQ ID NO: 630)
agtatggcccagccggccgaaattgtgttgacacagtctccag
(SEQ ID NO: 631)
tagaaggcacagtcgaggctgatcag
(SEQ ID NO: 632)
actgtcatatggaggtgcagctggtggagtctg
(SEQ ID NO: 633)
actgtctcgagtttaatttccactttggtgccgctgc
(SEQ ID NO: 634)
actgtcatatggaggtgcagctggtggagtctg
(SEQ ID NO: 635)
actgtaccggttttaatttccactttggtgccgctgc
(SEQ ID NO: 636)
cttcttcctcaggagcaagctcaccgtgg
(SEQ ID NO: 637)
gagccgtcggagtccagc
(SEQ ID NO: 638)
gcacctgaactcctgggg
(SEQ ID NO: 639)
tttaatttccactttggtgccg
(SEQ ID NO: 640)
cgcggctagcttaagcttggtaccgagggcca
(SEQ ID NO: 641)
cgcggcggccgcctgatcagcgggtttaaacttatc
MMP9
(DNA)
(SEQ ID NO: 642)
atgagcctctggcagcccctggtcctggtgctcctggtgctgggctgctgctttgctgcccccagacagcgccagtccacc
cttgtgctcttccctggagacctgagaaccaatctcaccgacaggcagctggcagaggaatacctgtaccgctatggttac
actcgggtggcagagatgcgtggagagtcgaaatctctggggcctgcgctgctgcttctccagaagcaactgtccctgccc
gagaccggtgagctggatagcgccacgctgaaggccatgcgaaccccacggtgcggggtcccagacctgggcagattccaa
acctttgagggcgacctcaagtggcaccaccacaacatcacctattggatccaaaactactcggaagacttgccgcgggcg
gtgattgacgacgcctttgcccgcgccttcgcactgtggagcgcggtgacgccgctcaccttcactcgcgtgtacagccgg
gacgcagacatcgtcatccagtttggtgtcgcggagcacggagacgggtatcccttcgacgggaaggacgggctcctggca
cacgcctttcctcctggccccggcattcagggagacgcccatttcgacgatgacgagttgtggtccctgggcaagggcgtc
gtggttccaactcggtttggaaacgcagatggcgcggcctgccacttccccttcatcttcgagggccgctcctactctgcc
tgcaccaccgacggtcgctccgacggcttgccctggtgcagtaccacggccaactacgacaccgacgaccggtttggcttc
tgccccagcgagagactctacacccaggacggcaatgctgatgggaaaccctgccagtttccattcatcttccaaggccaa
tcctactccgcctgcaccacggacggtcgctccgacggctaccgctggtgcgccaccaccgccaactacgaccgggacaag
ctcttcggcttctgcccgacccgagctgactcgacggtgatggggggcaactcggcgggggagctgtgcgtcttccccttc
actttcctgggtaaggagtactcgacctgtaccagcgagggccgcggagatgggcgcctctggtgcgctaccacctcgaac
tttgacagcgacaagaagtggggcttctgcccggaccaaggatacagtttgttcctcgtggcggcgcatgagttcggccac
gcgctgggcttagatcattcctcagtgccggaggcgctcatgtaccctatgtaccgcttcactgaggggccccccttgcat
aaggacgacgtgaatggcatccggcacctctatggtcctcgccctgaacctgagccacggcctccaaccaccaccacaccg
cagcccacggctcccccgacggtctgccccaccggaccccccactgtccacccctcagagcgccccacagctggccccaca
ggtcccccctcagctggccccacaggtccccccactgctggcccttctacggccactactgtgcctttgagtccggtggac
gatgcctgcaacgtgaacatcttcgacgccatcgcggagattgggaaccagctgtatttgttcaaggatgggaagtactgg
cgattctctgagggcagggggagccggccgcagggccccttccttatcgccgacaagtggcccgcgctgccccgcaagctg
gactcggtctttgaggagcggctctccaagaagcttttcttcttctctgggcgccaggtgtgggtgtacacaggcgcgtcg
gtgctgggcccgaggcgtctggacaagctgggcctgggagccgacgtggcccaggtgaccggggccctccggagtggcagg
gggaagatgctgctgttcagcgggcggcgcctctggaggttcgacgtgaaggcgcagatggtggatccccggagcgccagc
gaggtggaccggatgttccccggggtgcctttggacacgcacgacgtcttccagtaccgagagaaagcctatttctgccag
gaccgcttctactggcgcgtgagttcccggagtgagttgaaccaggtggaccaagtgggctacgtgacctatgacatcctg
cagtgccctgaggacgattacaaggatgacgacgataagtgataa
(amino acids)
(SEQ ID NO: 643)
MSLWQPLVLVLLVLGCCFAAPRQRQSTLVLFPGDLRTNLTDRQLAEEYLYRYGYTRVAEMRGESKSLGPALLLLQKQLSL
PETGELDSATLKAMRTPRCGVPDLGRFQTFEGDLKWHHHNITYWIQNYSEDLPRAVIDDAFARAFALWSAVTPLTFTRVY
SRDADIVIQFGVAEHGDGYPFDGKDGLLAHAFPPGPGIQGDAHFDDDELWSLGKGVVVPTRFGNADGAACHFPFIFEGRS
YSACTTDGRSDGLPWCSTTANYDTDDRFGFCPSERLYTQDGNADGKPCQFPFIFQGQSYSACTTDGRSDGYRWCATTANY
DRDKLFGFCPTRADSTVMGGNSAGELCVFPFTFLGKEYSTCTSEGRGDGRLWCATTSNFDSDKKWGFCPDQGYSLFLVAA
HEFGHALGLDHSSVPEALMYPMYRFTEGPPLHKDDVNGIRHLYGPRPEPEPRPPTTTTPQPTAPPTVCPTGPPTVHPSER
PTAGPTGPPSAGPTGPPTAGPSTATTVPLSPVDDACNVNIFDAIAEIGNQLYLFKDGKYWRFSEGRGSRPQGPFLIADKW
PALPRKLDSVFEERLSKKLFFFSGRQVWVYTGASVLGPRRLDKLGLGADVAQVTGALRSGRGKMLLFSGRRLWRFDVKAQ
MVDPRSASEVDRMFPGVPLDTHDVFQYREKAYFCQDRFYWRVSSRSELNQVDQVGYVTYDILQCPEDDYKDDDDK**
MMP9 catalytic domain
(DNA)
(SEQ ID NO: 644)
atgttccaaacctttgagggcgacctcaagtggcaccaccacaacatcacctattggatccaaaactactcggaagacttg
ccgcgggcggtgattgacgacgcctttgcccgcgccttcgcactgtggagcgcggtgacgccgctcaccttcactcgcgtg
tacagccgggacgcagacatcgtcatccagtttggtgtcgcggagcacggagacgggtatcccttcgacgggaaggacggg
ctcctggcacacgcctttcctcctggccccggcattcagggagacgcccatttcgacgatgacgagttgtggtccctgggc
aagggcgtcgtggttccaactcggtttggaaacgcagatggcgcggcctgccacttccccttcatcttcgagggccgctcc
tactctgcctgcaccaccgacggtcgctccgacggcttgccctggtgcagtaccacggccaactacgacaccgacgaccgg
tttggcttctgccccagcgagagactctacacccaggacggcaatgctgatgggaaaccctgccagtttccattcatcttc
caaggccaatcctactccgcctgcaccacggacggtcgctccgacggctaccgctggtgcgccaccaccgccaactacgac
cgggacaagctcttcggcttctgcccgacccgagctgactcgacggtgatggggggcaactcggcgggggagctgtgcgtc
ttccccttcactttcctgggtaaggagtactcgacctgtaccagcgagggccgcggagatgggcgcctctggtgcgctacc
acctcgaactttgacagcgacaagaagtggggcttctgcccggaccaaggatacagtttgttcctcgtggcggcgcatgag
ttcggccacgcgctgggcttagatcattcctcagtgccggaggcgctcatgtaccctatgtaccgcttcactgaggggccc
cccttgcataaggacgacgtgaatggcatccggcacctctatggtcctcgccctgaacctgattacaaggatgacgacgat
aagtgataa
(amino acids)
(SEQ ID NO: 645)
MFQTFEGDLKWHHHNITYWIQNYSEDLPRAVIDDAFARAFALWSAVTPLTFTRVYSRDADIVIQFGVAEHGDGYPFDGKD
GLLAHAFPPGPGIQGDAHFDDDELWSLGKGVVVPTRFGNADGAACHFPFIFEGRSYSACTTDGRSDGLPWCSTTANYDTD
DRFGFCPSERLYTQDGNADGKPCQFPFIFQGQSYSACTTDGRSDGYRWCATTANYDRDKLFGFCPTRADSTVMGGNSAGE
LCVFPFTFLGKEYSTCTSEGRGDGRLWCATTSNFDSDKKWGFCPDQGYSLFLVAAHEFGHALGLDHSSVPEALMYPMYRF
TEGPPLHKDDVNGIRHLYGPRPEPDYKDDDDK**
NFATc1 Promoter (NFATc1P)
(DNA)
(SEQ ID NO: 646)
aggcaggaggaagaggaaaggggcgcagggcgctcggggagcagagccgggggcccgcggtggccgcagaggccgggccgg
ggcgcagaggccgggcgagctggccgcgctctgggccgccgcctccggaactccctgcgcctggcgcgcggccaccgtgg
tcccggcaacggcattaaacagagggaaacagacccgggattccgtcacccgggcggggggataaggacggctttgagag
cagacaggaaaagggagcttttctgcatggggtgaaaaaattatttattgaaggaggaggaggcggcagcggaggaaggg
gaggggcgggaggaggaggaagagccggccgcccccgccccggccccggctcctcaggagccaagggcagcctcgccagg
tcggtcccgggctcgaggaccgcggctggggtcgaggggctcagtctcccacgtgaccggctgggcgcgccccgccagac
ccggcctcgggattccctcctcccggcgagtctccgcccgccccgtcctggaggtggggagaaggagggcggggcggggg
ggacggaaactctccccgccaaatcctggccccaggcctggggacactcgcggcgggaagatttggaggggaggggaggg
ggaggggcgtgggggcgcggcctcgctggagtccccctgaccccccgacccccgcccaccggcctgggcgtcctcccgcg
gcccctcctcccctcccggcgcccggtgctctggggcgcgtgccacgcctggctcggcgccgtaggggcccccgcaggta
gagacccctggaaatggcctcgacgccgcaggagcgaggcggccaccaccccgctaatccgggcacgtctctccaggccg
aggcctgcggtggaaaagccggggttccatttgtgctgagtcggggcggccgaatggagccaggcctcgggacgcgggac
ggacgggctctggccgcgcaccttcgcgggctctgcagcgcccgaccgcctcccccggcagggaggaggcgcttgtgggg
ggcacccacggggcacagtgatccctgggggtctgcggacctcctgggccccgcagcagacacgagtttagcctttgggt
ttagtttaaatcacataagggtgtcgtgcaatcgatttatggtttctacacaccagacactttaacctccaacccccccc
atccaagccaacaagaaaatgcggtgccgtgttggcagctgagctgcgcccgaagagacgcagggagacgtaagagagga
aagtgtgagtggccggggggcctccccccgtcagaagtcgcgcagtcgcgcccataaaacgccccctccgggcggctagg
gcaggtgagcgcgtccccgggcctccccacgccggcccctgccacagagccgtctaggtcgagcagatatttacagaata
aaaatgacaataactcgacgtcccgggacggccacgcaatctgttagtaatttagcgggatgggaatttcctttctaggg
cctgccagtgaagcgcttttccaaatttccacagcgggggaagcctgcgattttacataatgacttcagcatgccgggct
ttctcgacacccctccccggcccccggcccccgccccccgccccttttccagcagggccgggctccctccggacacccgc
gtggactcaggcgtcccgtctggcccgttcgcccccgtttcccccgccagccccagcgcccccctgcccggcccccggat
tccccgttcccgcccctacgcccccatcccctccccgtgcgcccctccccgtgcgcccccctccccgtgcgccccccctc
cccgtgcgcccccctccccgtgcgccccccctccccgggcgcccccctccccgggcgccccccctccccgtgcgcccccc
cctccccgtgcgccccccctccccgtgcgcgccccgcctcttgcgcccctgcccccaggcgagcggctgccgcggcgcgg
ggaggggcgggcgctcggcgactcgtccccggggccccgcgcgggcccgggcagcaggggcgtgatgtcacggcagggag
ggggcgcgggagccgccgggccggcggggaggcgggggaggtgttttccagctttaaaaaggcaggaggcagagcgcggc
cctgcgtcagagcgagactcagagg
NFATc1P-MMP9
(DNA)
(SEQ ID NO: 647)
aggcaggaggaagaggaaaggggcgcagggcgctcggggagcagagccgggggcccgcggtggccgcagaggccgggccgg
ggcgcagaggccgggcgagctggccgcgctctgggccgccgcctccggaactccctgcgcctggcgcgcggccaccgtgg
tcccggcaacggcattaaacagagggaaacagacccgggattccgtcacccgggcggggggataaggacggctttgagag
cagacaggaaaagggagcttttctgcatggggtgaaaaaattatttattgaaggaggaggaggcggcagcggaggaaggg
gaggggcgggaggaggaggaagagccggccgcccccgccccggccccggctcctcaggagccaagggcagcctcgccagg
tcggtcccgggctcgaggaccgcggctggggtcgaggggctcagtctcccacgtgaccggctgggcgcgccccgccagac
ccggcctcgggattccctcctcccggcgagtctccgcccgccccgtcctggaggtggggagaaggagggcggggcggggg
ggacggaaactctccccgccaaatcctggccccaggcctggggacactcgcggcgggaagatttggaggggaggggaggg
ggaggggcgtgggggcgcggcctcgctggagtccccctgaccccccgacccccgcccaccggcctgggcgtcctcccgcg
gcccctcctcccctcccggcgcccggtgctctggggcgcgtgccacgcctggctcggcgccgtaggggcccccgcaggta
gagacccctggaaatggcctcgacgccgcaggagcgaggcggccaccaccccgctaatccgggcacgtctctccaggccg
aggcctgcggtggaaaagccggggttccatttgtgctgagtcggggcggccgaatggagccaggcctcgggacgcgggac
ggacgggctctggccgcgcaccttcgcgggctctgcagcgcccgaccgcctcccccggcagggaggaggcgcttgtgggg
ggcacccacggggcacagtgatccctgggggtctgcggacctcctgggccccgcagcagacacgagtttagcctttgggt
ttagtttaaatcacataagggtgtcgtgcaatcgatttatggtttctacacaccagacactttaacctccaacccccccc
atccaagccaacaagaaaatgcggtgccgtgttggcagctgagctgcgcccgaagagacgcagggagacgtaagagagga
aagtgtgagtggccggggggcctccccccgtcagaagtcgcgcagtcgcgcccataaaacgccccctccgggcggctagg
gcaggtgagcgcgtccccgggcctccccacgccggcccctgccacagagccgtctaggtcgagcagatatttacagaata
aaaatgacaataactcgacgtcccgggacggccacgcaatctgttagtaatttagcgggatgggaatttcctttctaggg
cctgccagtgaagcgcttttccaaatttccacagcgggggaagcctgcgattttacataatgacttcagcatgccgggct
ttctcgacacccctccccggcccccggcccccgccccccgccccttttccagcagggccgggctccctccggacacccgc
gtggactcaggcgtcccgtctggcccgttcgcccccgtttcccccgccagccccagcgcccccctgcccggcccccggat
tccccgttcccgcccctacgcccccatcccctccccgtgcgcccctccccgtgcgcccccctccccgtgcgccccccctc
cccgtgcgcccccctccccgtgcgccccccctccccgggcgcccccctccccgggcgccccccctccccgtgcgcccccc
cctccccgtgcgccccccctccccgtgcgcgccccgcctcttgcgcccctgcccccaggcgagcggctgccgcggcgcgg
ggaggggcgggcgctcggcgactcgtccccggggccccgcgcgggcccgggcagcaggggcgtgatgtcacggcagggag
ggggcgcgggagccgccgggccggcggggaggcgggggaggtgttttccagctttaaaaaggcaggaggcagagcgcggc
cctgcgtcagagcgagactcagaggtctagagccaccatgagcctctggcagcccctggtcctggtgctcctggtgctgg
gctgctgctttgctgcccccagacagcgccagtccacccttgtgctcttccctggagacctgagaaccaatctcaccgaca
ggcagctggcagaggaatacctgtaccgctatggttacactcgggtggcagagatgcgtggagagtcgaaatctctggggc
ctgcgctgctgcttctccagaagcaactgtccctgcccgagaccggtgagctggatagcgccacgctgaaggccatgcgaa
ccccacggtgcggggtcccagacctgggcagattccaaacctttgagggcgacctcaagtggcaccaccacaacatcacct
attggatccaaaactactcggaagacttgccgcgggcggtgattgacgacgcctttgcccgcgccttcgcactgtggagcg
cggtgacgccgctcaccttcactcgcgtgtacagccgggacgcagacatcgtcatccagtttggtgtcgcggagcacggag
acgggtatcccttcgacgggaaggacgggctcctggcacacgcctttcctcctggccccggcattcagggagacgcccatt
tcgacgatgacgagttgtggtccctgggcaagggcgtcgtggttccaactcggtttggaaacgcagatggcgcggcctgcc
acttccccttcatcttcgagggccgctcctactctgcctgcaccaccgacggtcgctccgacggcttgccctggtgcagta
ccacggccaactacgacaccgacgaccggtttggcttctgccccagcgagagactctacacccaggacggcaatgctgatg
ggaaaccctgccagtttccattcatcttccaaggccaatcctactccgcctgcaccacggacggtcgctccgacggctacc
gctggtgcgccaccaccgccaactacgaccgggacaagctcttcggcttctgcccgacccgagctgactcgacggtgatgg
ggggcaactcggcgggggagctgtgcgtcttccccttcactttcctgggtaaggagtactcgacctgtaccagcgagggcc
gcggagatgggcgcctctggtgcgctaccacctcgaactttgacagcgacaagaagtggggcttctgcccggaccaaggat
acagtttgttcctcgtggcggcgcatgagttcggccacgcgctgggcttagatcattcctcagtgccggaggcgctcatgt
accctatgtaccgcttcactgaggggccccccttgcataaggacgacgtgaatggcatccggcacctctatggtcctcgcc
ctgaacctgagccacggcctccaaccaccaccacaccgcagcccacggctcccccgacggtctgccccaccggacccccca
ctgtccacccctcagagcgccccacagctggccccacaggtcccccctcagctggccccacaggtccccccactgctggcc
cttctacggccactactgtgcctttgagtccggtggacgatgcctgcaacgtgaacatcttcgacgccatcgcggagattg
ggaaccagctgtatttgttcaaggatgggaagtactggcgattctctgagggcagggggagccggccgcagggccccttcc
ttatcgccgacaagtggcccgcgctgccccgcaagctggactcggtctttgaggagcggctctccaagaagcttttcttct
tctctgggcgccaggtgtgggtgtacacaggcgcgtcggtgctgggcccgaggcgtctggacaagctgggcctgggagccg
acgtggcccaggtgaccggggccctccggagtggcagggggaagatgctgctgttcagcgggcggcgcctctggaggttcg
acgtgaaggcgcagatggtggatccccggagcgccagcgaggtggaccggatgttccccggggtgcctttggacacgcacg
acgtcttccagtaccgagagaaagcctatttctgccaggaccgcttctactggcgcgtgagttcccggagtgagttgaacc
aggtggaccaagtgggctacgtgacctatgacatcctgcagtgccctgaggacgattacaaggatgacgacgataagtgat
aa
NFATc1P-MMP9cat
(DNA)
(SEQ ID NO: 648)
aggcaggaggaagaggaaaggggcgcagggcgctcggggagcagagccgggggcccgcggtggccgcagaggccgggccgg
ggcgcagaggccgggcgagctggccgcgctctgggccgccgcctccggaactccctgcgcctggcgcgcggccaccgtgg
tcccggcaacggcattaaacagagggaaacagacccgggattccgtcacccgggcggggggataaggacggctttgagag
cagacaggaaaagggagcttttctgcatggggtgaaaaaattatttattgaaggaggaggaggcggcagcggaggaaggg
gaggggcgggaggaggaggaagagccggccgcccccgccccggccccggctcctcaggagccaagggcagcctcgccagg
tcggtcccgggctcgaggaccgcggctggggtcgaggggctcagtctcccacgtgaccggctgggcgcgccccgccagac
ccggcctcgggattccctcctcccggcgagtctccgcccgccccgtcctggaggtggggagaaggagggcggggcggggg
ggacggaaactctccccgccaaatcctggccccaggcctggggacactcgcggcgggaagatttggaggggaggggaggg
ggaggggcgtgggggcgcggcctcgctggagtccccctgaccccccgacccccgcccaccggcctgggcgtcctcccgcg
gcccctcctcccctcccggcgcccggtgctctggggcgcgtgccacgcctggctcggcgccgtaggggcccccgcaggta
gagacccctggaaatggcctcgacgccgcaggagcgaggcggccaccaccccgctaatccgggcacgtctctccaggccg
aggcctgcggtggaaaagccggggttccatttgtgctgagtcggggcggccgaatggagccaggcctcgggacgcgggac
ggacgggctctggccgcgcaccttcgcgggctctgcagcgcccgaccgcctcccccggcagggaggaggcgcttgtgggg
ggcacccacggggcacagtgatccctgggggtctgcggacctcctgggccccgcagcagacacgagtttagcctttgggt
ttagtttaaatcacataagggtgtcgtgcaatcgatttatggtttctacacaccagacactttaacctccaacccccccc
atccaagccaacaagaaaatgcggtgccgtgttggcagctgagctgcgcccgaagagacgcagggagacgtaagagagga
aagtgtgagtggccggggggcctccccccgtcagaagtcgcgcagtcgcgcccataaaacgccccctccgggcggctagg
gcaggtgagcgcgtccccgggcctccccacgccggcccctgccacagagccgtctaggtcgagcagatatttacagaata
aaaatgacaataactcgacgtcccgggacggccacgcaatctgttagtaatttagcgggatgggaatttcctttctaggg
cctgccagtgaagcgcttttccaaatttccacagcgggggaagcctgcgattttacataatgacttcagcatgccgggct
ttctcgacacccctccccggcccccggcccccgccccccgccccttttccagcagggccgggctccctccggacacccgc
gtggactcaggcgtcccgtctggcccgttcgcccccgtttcccccgccagccccagcgcccccctgcccggcccccggat
tccccgttcccgcccctacgcccccatcccctccccgtgcgcccctccccgtgcgcccccctccccgtgcgccccccctc
cccgtgcgcccccctccccgtgcgccccccctccccgggcgcccccctccccgggcgccccccctccccgtgcgcccccc
cctccccgtgcgccccccctccccgtgcgcgccccgcctcttgcgcccctgcccccaggcgagcggctgccgcggcgcgg
ggaggggcgggcgctcggcgactcgtccccggggccccgcgcgggcccgggcagcaggggcgtgatgtcacggcagggag
ggggcgcgggagccgccgggccggcggggaggcgggggaggtgttttccagctttaaaaaggcaggaggcagagcgcggc
cctgcgtcagagcgagactcagaggtctagagccaccatgttccaaacctttgagggcgacctcaagtggcaccaccaca
acatcacctattggatccaaaactactcggaagacttgccgcgggcggtgattgacgacgcctttgcccgcgccttcgcac
tgtggagcgcggtgacgccgctcaccttcactcgcgtgtacagccgggacgcagacatcgtcatccagtttggtgtcgcgg
agcacggagacgggtatcccttcgacgggaaggacgggctcctggcacacgcctttcctcctggccccggcattcagggag
acgcccatttcgacgatgacgagttgtggtccctgggcaagggcgtcgtggttccaactcggtttggaaacgcagatggcg
cggcctgccacttccccttcatcttcgagggccgctcctactctgcctgcaccaccgacggtcgctccgacggcttgccct
ggtgcagtaccacggccaactacgacaccgacgaccggtttggcttctgccccagcgagagactctacacccaggacggca
atgctgatgggaaaccctgccagtttccattcatcttccaaggccaatcctactccgcctgcaccacggacggtcgctccg
acggctaccgctggtgcgccaccaccgccaactacgaccgggacaagctcttcggcttctgcccgacccgagctgactcga
cggtgatggggggcaactcggcgggggagctgtgcgtcttccccttcactttcctgggtaaggagtactcgacctgtacca
gcgagggccgcggagatgggcgcctctggtgcgctaccacctcgaactttgacagcgacaagaagtggggcttctgcccgg
accaaggatacagtttgttcctcgtggcggcgcatgagttcggccacgcgctgggcttagatcattcctcagtgccggagg
cgctcatgtaccctatgtaccgcttcactgaggggccccccttgcataaggacgacgtgaatggcatccggcacctctatg
gtcctcgccctgaacctgattacaaggatgacgacgataagtgataa
NFAT response element
(DNA)
(SEQ ID NO: 649)
ggaggaaaaactgtttcatacagaaggcgt
NFAT response element repeats
(DNA)
(SEQ ID NO: 650)
ggaggaaaaactgtttcatacagaaggcgtggaggaaaaactgtttcatacagaaggcgtggaggaaaaactgtttcatac
agaaggcgt
CMV minimal promoter
(DNA)
(SEQ ID NO: 651)
aggtaggcgtgtacggtgggaggtctatataagcagagctggtttagtgaaccgtcagatc
NFATREmCMV-MMP9
(DNA)
(SEQ ID NO: 652)
ggaggaaaaactgtttcatacagaaggcgtggaggaaaaactgtttcatacagaaggcgtggaggaaaaactgtttcatac
agaaggcgtagatctagactcaggtaggcgtgtacggtgggaggtctatataagcagagctggtttagtgaaccgtcagat
ctctagagccaccatgagcctctggcagcccctggtcctggtgctcctggtgctgggctgctgctttgctgcccccagaca
gcgccagtccacccttgtgctcttccctggagacctgagaaccaatctcaccgacaggcagctggcagaggaatacctgta
ccgctatggttacactcgggtggcagagatgcgtggagagtcgaaatctctggggcctgcgctgctgcttctccagaagca
actgtccctgcccgagaccggtgagctggatagcgccacgctgaaggccatgcgaaccccacggtgcggggtcccagacct
gggcagattccaaacctttgagggcgacctcaagtggcaccaccacaacatcacctattggatccaaaactactcggaaga
cttgccgcgggcggtgattgacgacgcctttgcccgcgccttcgcactgtggagcgcggtgacgccgctcaccttcactcg
cgtgtacagccgggacgcagacatcgtcatccagtttggtgtcgcggagcacggagacgggtatcccttcgacgggaagga
cgggctcctggcacacgcctttcctcctggccccggcattcagggagacgcccatttcgacgatgacgagttgtggtccct
gggcaagggcgtcgtggttccaactcggtttggaaacgcagatggcgcggcctgccacttccccttcatcttcgagggccg
ctcctactctgcctgcaccaccgacggtcgctccgacggcttgccctggtgcagtaccacggccaactacgacaccgacga
ccggtttggcttctgccccagcgagagactctacacccaggacggcaatgctgatgggaaaccctgccagtttccattcat
cttccaaggccaatcctactccgcctgcaccacggacggtcgctccgacggctaccgctggtgcgccaccaccgccaacta
cgaccgggacaagctcttcggcttctgcccgacccgagctgactcgacggtgatggggggcaactcggcgggggagctgtg
cgtcttccccttcactttcctgggtaaggagtactcgacctgtaccagcgagggccgcggagatgggcgcctctggtgcgc
taccacctcgaactttgacagcgacaagaagtggggcttctgcccggaccaaggatacagtttgttcctcgtggcggcgca
tgagttcggccacgcgctgggcttagatcattcctcagtgccggaggcgctcatgtaccctatgtaccgcttcactgaggg
gccccccttgcataaggacgacgtgaatggcatccggcacctctatggtcctcgccctgaacctgagccacggcctccaac
caccaccacaccgcagcccacggctcccccgacggtctgccccaccggaccccccactgtccacccctcagagcgccccac
agctggccccacaggtcccccctcagctggccccacaggtccccccactgctggcccttctacggccactactgtgccttt
gagtccggtggacgatgcctgcaacgtgaacatcttcgacgccatcgcggagattgggaaccagctgtatttgttcaagga
tgggaagtactggcgattctctgagggcagggggagccggccgcagggccccttccttatcgccgacaagtggcccgcgct
gccccgcaagctggactcggtctttgaggagcggctctccaagaagcttttcttcttctctgggcgccaggtgtgggtgta
cacaggcgcgtcggtgctgggcccgaggcgtctggacaagctgggcctgggagccgacgtggcccaggtgaccggggccct
ccggagtggcagggggaagatgctgctgttcagcgggcggcgcctctggaggttcgacgtgaaggcgcagatggtggatcc
ccggagcgccagcgaggtggaccggatgttccccggggtgcctttggacacgcacgacgtcttccagtaccgagagaaagc
ctatttctgccaggaccgcttctactggcgcgtgagttcccggagtgagttgaaccaggtggaccaagtgggctacgtgac
ctatgacatcctgcagtgccctgaggacgattacaaggatgacgacgataagtgataa
NFATREmCMV-MMP9cat
(DNA)
(SEQ ID NO: 653)
ggaggaaaaactgtttcatacagaaggcgtggaggaaaaactgtttcatacagaaggcgtggaggaaaaactgtttcatac
agaaggcgtagatctagactcaggtaggcgtgtacggtgggaggtctatataagcagagctggtttagtgaaccgtcagat
ctctagagccaccatgttccaaacctttgagggcgacctcaagtggcaccaccacaacatcacctattggatccaaaacta
ctcggaagacttgccgcgggcggtgattgacgacgcctttgcccgcgccttcgcactgtggagcgcggtgacgccgctcac
cttcactcgcgtgtacagccgggacgcagacatcgtcatccagtttggtgtcgcggagcacggagacgggtatcccttcga
cgggaaggacgggctcctggcacacgcctttcctcctggccccggcattcagggagacgcccatttcgacgatgacgagtt
gtggtccctgggcaagggcgtcgtggttccaactcggtttggaaacgcagatggcgcggcctgccacttccccttcatctt
cgagggccgctcctactctgcctgcaccaccgacggtcgctccgacggcttgccctggtgcagtaccacggccaactacga
caccgacgaccggtttggcttctgccccagcgagagactctacacccaggacggcaatgctgatgggaaaccctgccagtt
tccattcatcttccaaggccaatcctactccgcctgcaccacggacggtcgctccgacggctaccgctggtgcgccaccac
cgccaactacgaccgggacaagctcttcggcttctgcccgacccgagctgactcgacggtgatggggggcaactcggcggg
ggagctgtgcgtcttccccttcactttcctgggtaaggagtactcgacctgtaccagcgagggccgcggagatgggcgcct
ctggtgcgctaccacctcgaactttgacagcgacaagaagtggggcttctgcccggaccaaggatacagtttgttcctcgt
ggcggcgcatgagttcggccacgcgctgggcttagatcattcctcagtgccggaggcgctcatgtaccctatgtaccgctt
cactgaggggccccccttgcataaggacgacgtgaatggcatccggcacctctatggtcctcgccctgaacctgattacaa
ggatgacgacgataagtgataa
C2 scFir
(DNA)
(SEQ ID NO: 654)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaact
(amino acids)
(SEQ ID NO: 655)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRT
CD8 transmembrane domain
(DNA)
(SEQ ID NO: 656)
atctacatctgggcgcccttggccgggacttgtggggtccttctcctgtcactggttatcaccctttactgc
(amino acids)
(SEQ ID NO: 657)
IYIWAPLAGTCGVLLLSLVITLYC
4-1BB domain
(DNA)
(SEQ ID NO: 658)
aaacggggcagaaagaaactcctgtatatattcaaacaaccatttatgagaccagtacaaactactcaagaggaagatggc
tgtagctgccgatttccagaagaagaagaaggaggatgtgaactg
(amino acids)
(SEQ ID NO: 659)
KRGRKKLLYIFKQPFMRPVQTTQEEDGCSCRFPEEEEGGCEL
CD3 zeta domain
(DNA)
(SEQ ID NO: 660)
agagtgaagttcagcaggagcgcagacgcccccgcgtacaagcagggccagaaccagctctataacgagctcaatctagga
cgaagagaggagtacgatgttttggacaagagacgtggccgggaccctgagatggggggaaagccgagaaggaagaaccct
caggaaggcctgtacaatgaactgcagaaagataagatggcggaggcctacagtgagattgggatgaaaggcgagcgccgg
aggggcaaggggcacgatggcctttaccagggtctcagtacagccaccaaggacacctacgacgcccttcacatgcaggcc
ctgccccctcgc
(amino acids)
(SEQ ID NO: 661)
RVKFSRSADAPAYKQGQNQLYNELNLGRREEYDVLDKRRGRDPEMGGKPRRKNPQEGLYNELQKDKMAEAYSEIGMKGER
RRGKGHDGLYQGLSTATKDTYDALHMQALPPR
Human IgG1 Fc linker
(DNA)
(SEQ ID NO: 662)
gagcccaaatcttgtgacaaaactcacacatgcccaccgtgcccagcacctgaactcctggggggaccgtcagtcttcctc
ttccccccaaaacccaaggacaccctcatgatctcccggacccctgaggtcacatgcgtggtggtggacgtgagccacgaa
gaccctgaggtcaagttcaactggtacgtggacggcgtggaggtgcataatgccaagacaaagccgcgggaggagcagtac
aacagcacgtaccgtgtggtcagcgtcctcaccgtcctgcaccaggactggctgaatggcaaggagtacaagtgcaaggtc
tccaacaaagccctcccagcccccatcgagaaaaccatctccaaagccaaagggcagccccgagaaccacaggtgtacacc
ctgcccccatcccgggaggagatgaccaagaaccaggtcagcctgacctgcctggtcaaaggcttctatcccagcgacatc
gccgtggagtgggagagcaatgggcagccggagaacaactacaagaccacgcctcccgtgctggactccgacggctccttc
ttcctctacagcaagctcaccgtggacaagagcaggtggcagcaggggaacgtcttctcatgctccgtgatgcatgaggct
ctgcacaaccactacacgcagaagagcctctccctgtctccgggtaaa
(amino acids)
(SEQ ID NO: 663)
EPKSCDKTHTCPPCPAPELLGGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVFSCSVMHEALHNHYTQKSLSLSPGK
C2 CAR FC linker
(DNA)
(SEQ ID NO: 664)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaactgagcccaaatcttgtgacaaaactcacacatgcccaccgtgcccagcacctgaactcctgggg
ggaccgtcagtcttcctcttccccccaaaacccaaggacaccctcatgatctcccggacccctgaggtcacatgcgtggtg
gtggacgtgagccacgaagaccctgaggtcaagttcaactggtacgtggacggcgtggaggtgcataatgccaagacaaag
ccgcgggaggagcagtacaacagcacgtaccgtgtggtcagcgtcctcaccgtcctgcaccaggactggctgaatggcaag
gagtacaagtgcaaggtctccaacaaagccctcccagcccccatcgagaaaaccatctccaaagccaaagggcagccccga
gaaccacaggtgtacaccctgcccccatcccgggaggagatgaccaagaaccaggtcagcctgacctgcctggtcaaaggc
ttctatcccagcgacatcgccgtggagtgggagagcaatgggcagccggagaacaactacaagaccacgcctcccgtgctg
gactccgacggctccttcttcctctacagcaagctcaccgtggacaagagcaggtggcagcaggggaacgtcttctcatgc
tccgtgatgcatgaggctctgcacaaccactacacgcagaagagcctctccctgtctccgggtaaaatctacatctgggcg
cccttggccgggacttgtggggtccttctcctgtcactggttatcaccctttactgcaaacggggcagaaagaaactcctg
tatatattcaaacaaccatttatgagaccagtacaaactactcaagaggaagatggctgtagctgccgatttccagaagaa
gaagaaggaggatgtgaactgagagtgaagttcagcaggagcgcagacgcccccgcgtacaagcagggccagaaccagctc
tataacgagctcaatctaggacgaagagaggagtacgatgttttggacaagagacgtggccgggaccctgagatgggggga
aagccgagaaggaagaaccctcaggaaggcctgtacaatgaactgcagaaagataagatggcggaggcctacagtgagatt
gggatgaaaggcgagcgccggaggggcaaggggcacgatggcctttaccagggtctcagtacagccaccaaggacacctac
gacgcccttcacatgcaggccctgccccctcgctgataa
(amino acids)
(SEQ ID NO: 665)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRTEPKSCDKTHTCPPCPAPELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKSRWQQGNVFSCSVMHEALHNHYTQKSLSLSPG
KIYIWAPLAGTCGVLLLSLVITLYCKRGRKKLLYIFKQPFMRPVQTTQEEDGCSCRFPEEEEGGCELRVKFSRSADAPAY
KQGQNQLYNELNLGRREEYDVLDKRRGRDPEMGGKPRRKNPQEGLYNELQKDKMAEAYSEIGMKGERRRGKGHDGLYQGL
STATKDTYDALHMQALPPR**
IgD/Fc linker
(DNA)
(SEQ ID NO: 666)
gagtctccaaaggcacaggcctcctcagtgcccactgcacaaccccaagcagagggcagcctcgccaaggcaaccacagcc
ccagccaccacccgtaacacaggaagaggcggcgaagagaagaaaaaggagaaggagaaagaggaacaagaagagagagag
acaaagacaccagagcccaaatcttgtgacaaaactcacacatgcccaccgtgcccagcacctgaactcctggggggaccg
tcagtcttcctcttccccccaaaacccaaggacaccctcatgatctcccggacccctgaggtcacatgcgtggtggtggac
gtgagccacgaagaccctgaggtcaagttcaactggtacgtggacggcgtggaggtgcataatgccaagacaaagccgcgg
gaggagcagtacaacagcacgtaccgtgtggtcagcgtcctcaccgtcctgcaccaggactggctgaatggcaaggagtac
aagtgcaaggtctccaacaaagccctcccagcccccatcgagaaaaccatctccaaagccaaagggcagccccgagaacca
caggtgtacaccctgcccccatcccgggaggagatgaccaagaaccaggtcagcctgacctgcctggtcaaaggcttctat
cccagcgacatcgccgtggagtgggagagcaatgggcagccggagaacaactacaagaccacgcctcccgtgctggactcc
gacggctccttcttcctctacagcaagctcaccgtggacaagagcaggtggcagcaggggaacgtcttctcatgctccgtg
atgcatgaggctctgcacaaccactacacgcagaagagcctctccctgtctccgggtaaa
(amino acids)
(SEQ ID NO: 667)
ESPKAQASSVPTAQPQAEGSLAKATTAPATTRNTGRGGEEKKKEKEKEEQEERETKTPEPKSCDKTHTCPPCPAPELLGG
PSVFLFPPKPKDTLMISRTPEVTCVVVDVSHEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
C2 CAR IgD/FC linker
(DNA)
(SEQ ID NO: 668)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaactgagtctccaaaggcacaggcctcctcagtgcccactgcacaaccccaagcagagggcagcctc
gccaaggcaaccacagccccagccaccacccgtaacacaggaagaggcggcgaagagaagaaaaaggagaaggagaaagag
gaacaagaagagagagagacaaagacaccagagcccaaatcttgtgacaaaactcacacatgcccaccgtgcccagcacct
gaactcctggggggaccgtcagtcttcctcttccccccaaaacccaaggacaccctcatgatctcccggacccctgaggtc
acatgcgtggtggtggacgtgagccacgaagaccctgaggtcaagttcaactggtacgtggacggcgtggaggtgcataat
gccaagacaaagccgcgggaggagcagtacaacagcacgtaccgtgtggtcagcgtcctcaccgtcctgcaccaggactgg
ctgaatggcaaggagtacaagtgcaaggtctccaacaaagccctcccagcccccatcgagaaaaccatctccaaagccaaa
gggcagccccgagaaccacaggtgtacaccctgcccccatcccgggaggagatgaccaagaaccaggtcagcctgacctgc
ctggtcaaaggcttctatcccagcgacatcgccgtggagtgggagagcaatgggcagccggagaacaactacaagaccacg
cctcccgtgctggactccgacggctccttcttcctctacagcaagctcaccgtggacaagagcaggtggcagcaggggaac
gtcttctcatgctccgtgatgcatgaggctctgcacaaccactacacgcagaagagcctctccctgtctccgggtaaaatc
tacatctgggcgcccttggccgggacttgtggggtccttctcctgtcactggttatcaccctttactgcaaacggggcaga
aagaaactcctgtatatattcaaacaaccatttatgagaccagtacaaactactcaagaggaagatggctgtagctgccga
tttccagaagaagaagaaggaggatgtgaactgagagtgaagttcagcaggagcgcagacgcccccgcgtacaagcagggc
cagaaccagctctataacgagctcaatctaggacgaagagaggagtacgatgttttggacaagagacgtggccgggaccct
gagatggggggaaagccgagaaggaagaaccctcaggaaggcctgtacaatgaactgcagaaagataagatggcggaggcc
tacagtgagattgggatgaaaggcgagcgccggaggggcaaggggcacgatggcctttaccagggtctcagtacagccacc
aaggacacctacgacgcccttcacatgcaggccctgccccctcgctgataa
(amino acids)
(SEQ ID NO: 669)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRTESPKAQASSVPTAQPQAEGSLAKATTAPATTRNTGRGGEEKKKEKEKEEQEERETKTPEPKSCDKTHTCPP
CPAPELLGGPSVFLFPPKPKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGKIYIWAPLAGTCGVLLLSLVIT
LYCKRGRKKLLYIFKQPFMRPVQTTQEEDGCSCRFPEEEEGGCELRVKFSRSADAPAYKQGQNQLYNELNLGRREEYDVL
DKRRGRDPEMGGKPRRKNPQEGLYNELQKDKMAEAYSEIGMKGERRRGKGHDGLYQGLSTATKDTYDALHMQALPPR**
Fc hingeless Y407R linker
(DNA)
(SEQ ID NO: 670)
gcacctgaactcctggggggaccgtcagtcttcctcttccccccaaaacccaaggacaccctcatgatctcccggacccct
gaggtcacatgcgtggtggtggacgtgagccacgaagaccctgaggtcaagttcaactggtacgtggacggcgtggaggtg
cataatgccaagacaaagccgcgggaggagcagtacaacagcacgtaccgtgtggtcagcgtcctcaccgtcctgcaccag
gactggctgaatggcaaggagtacaagtgcaaggtctccaacaaagccctcccagcccccatcgagaaaaccatctccaaa
gccaaagggcagccccgagaaccacaggtgtacaccctgcccccatcccgggaggagatgaccaagaaccaggtcagcctg
acctgcctggtcaaaggcttctatcccagcgacatcgccgtggagtgggagagcaatgggcagccggagaacaactacaag
accacgcctcccgtgctggactccgacggctccttcttcctcaggagcaagctcaccgtggacaagagcaggtggcagcag
gggaacgtcttctcatgctccgtgatgcatgaggctctgcacaaccactacacgcagaagagcctctccctgtctccgggt
aaa
(amino acids)
(SEQ ID NO: 671)
APELLGGPSVFLFPPKPKDTLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMTKNQVSLTCLVKGFYPSDIAVEWESNGQPENN
YKTTPPVLDSDGSFFLRSKLTVDKSRWQQGNVFSCSVMHEALHNHYTQKSLSLSPGK
C2 CAR FCHingeless/Y407R linker
(DNA)
(SEQ ID NO: 672)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaactgcacctgaactcctggggggaccgtcagtcttcctcttccccccaaaacccaaggacaccctc
atgatctcccggacccctgaggtcacatgcgtggtggtggacgtgagccacgaagaccctgaggtcaagttcaactggtac
gtggacggcgtggaggtgcataatgccaagacaaagccgcgggaggagcagtacaacagcacgtaccgtgtggtcagcgtc
ctcaccgtcctgcaccaggactggctgaatggcaaggagtacaagtgcaaggtctccaacaaagccctcccagcccccatc
gagaaaaccatctccaaagccaaagggcagccccgagaaccacaggtgtacaccctgcccccatcccgggaggagatgacc
aagaaccaggtcagcctgacctgcctggtcaaaggcttctatcccagcgacatcgccgtggagtgggagagcaatgggcag
ccggagaacaactacaagaccacgcctcccgtgctggactccgacggctccttcttcctcaggagcaagctcaccgtggac
aagagcaggtggcagcaggggaacgtcttctcatgctccgtgatgcatgaggctctgcacaaccactacacgcagaagagc
ctctccctgtctccgggtaaaatctacatctgggcgcccttggccgggacttgtggggtccttctcctgtcactggttatc
accctttactgcaaacggggcagaaagaaactcctgtatatattcaaacaaccatttatgagaccagtacaaactactcaa
gaggaagatggctgtagctgccgatttccagaagaagaagaaggaggatgtgaactgagagtgaagttcagcaggagcgca
gacgcccccgcgtacaagcagggccagaaccagctctataacgagctcaatctaggacgaagagaggagtacgatgttttg
gacaagagacgtggccgggaccctgagatggggggaaagccgagaaggaagaaccctcaggaaggcctgtacaatgaactg
cagaaagataagatggcggaggcctacagtgagattgggatgaaaggcgagcgccggaggggcaaggggcacgatggcctt
taccagggtctcagtacagccaccaaggacacctacgacgcccttcacatgcaggccctgccccctcgctgataa
(amino acids)
(SEQ ID NO: 673)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRTAPELLGGPSVFLFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSREEMTKNQVSLTCLVKGFYPSDIAVEW
ESNGQPENNYKTTPPVLDSDGSFFLRSKLTVDKSRWQQGNVFSCSVMHEALHNHYTQKSLSLSPGKIYIWAPLAGTCGVL
LLSLVITLYCKRGRKKLLYIFKQPFMRPVQTTQEEDGCSCRFPEEEEGGCELRVKFSRSADAPAYKQGQNQLYNELNLGR
REEYDVLDKRRGRDPEMGGKPRRKNPQEGLYNELQKDKMAEAYSEIGMKGERRRGKGHDGLYQGLSTATKDTYDALHMQA
LPPR**
IgD/FCHingeless/Y407R linker
(DNA)
(SEQ ID NO: 674)
gagtctccaaaggcacaggcctcctcagtgcccactgcacaaccccaagcagagggcagcctcgccaaggcaaccacagcc
ccagccaccacccgtaacacaggaagaggcggcgaagagaagaaaaaggagaaggagaaagaggaacaagaagagagagag
acaaagacaccagcacctgaactcctggggggaccgtcagtcttcctcttccccccaaaacccaaggacaccctcatgatc
tcccggacccctgaggtcacatgcgtggtggtggacgtgagccacgaagaccctgaggtcaagttcaactggtacgtggac
ggcgtggaggtgcataatgccaagacaaagccgcgggaggagcagtacaacagcacgtaccgtgtggtcagcgtcctcacc
gtcctgcaccaggactggctgaatggcaaggagtacaagtgcaaggtctccaacaaagccctcccagcccccatcgagaaa
accatctccaaagccaaagggcagccccgagaaccacaggtgtacaccctgcccccatcccgggaggagatgaccaagaac
caggtcagcctgacctgcctggtcaaaggcttctatcccagcgacatcgccgtggagtgggagagcaatgggcagccggag
aacaactacaagaccacgcctcccgtgctggactccgacggctccttcttcctcaggagcaagctcaccgtggacaagagc
aggtggcagcaggggaacgtcttctcatgctccgtgatgcatgaggctctgcacaaccactacacgcagaagagcctctcc
ctgtctccgggtaaa
(amino acids)
(SEQ ID NO: 675)
ESPKAQASSVPTAQPQAEGSLAKATTAPATTRNTGRGGEEKKKEKEKEEQEERETKTPAPELLGGPSVFLFPPKPKDTLM
ISRTPEVTCVVVDVSHEDPEVKFNWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNYKTTPPVLDSDGSFFLRSKLTV
DKSRWQQGNVFSCSVMHEALHNHYTQKSLSLSPGK
C2 CAR IgD/FCHingeless/Y407R linker
(DNA)
(SEQ ID NO: 676)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaactgagtctccaaaggcacaggcctcctcagtgcccactgcacaaccccaagcagagggcagcctc
gccaaggcaaccacagccccagccaccacccgtaacacaggaagaggcggcgaagagaagaaaaaggagaaggagaaagag
gaacaagaagagagagagacaaagacaccagcacctgaactcctggggggaccgtcagtcttcctcttccccccaaaaccc
aaggacaccctcatgatctcccggacccctgaggtcacatgcgtggtggtggacgtgagccacgaagaccctgaggtcaag
ttcaactggtacgtggacggcgtggaggtgcataatgccaagacaaagccgcgggaggagcagtacaacagcacgtaccgt
gtggtcagcgtcctcaccgtcctgcaccaggactggctgaatggcaaggagtacaagtgcaaggtctccaacaaagccctc
ccagcccccatcgagaaaaccatctccaaagccaaagggcagccccgagaaccacaggtgtacaccctgcccccatcccgg
gaggagatgaccaagaaccaggtcagcctgacctgcctggtcaaaggcttctatcccagcgacatcgccgtggagtgggag
agcaatgggcagccggagaacaactacaagaccacgcctcccgtgctggactccgacggctccttcttcctcaggagcaag
ctcaccgtggacaagagcaggtggcagcaggggaacgtcttctcatgctccgtgatgcatgaggctctgcacaaccactac
acgcagaagagcctctccctgtctccgggtaaaatctacatctgggcgcccttggccgggacttgtggggtccttctcctg
tcactggttatcaccctttactgcaaacggggcagaaagaaactcctgtatatattcaaacaaccatttatgagaccagta
caaactactcaagaggaagatggctgtagctgccgatttccagaagaagaagaaggaggatgtgaactgagagtgaagttc
agcaggagcgcagacgcccccgcgtacaagcagggccagaaccagctctataacgagctcaatctaggacgaagagaggag
tacgatgttttggacaagagacgtggccgggaccctgagatggggggaaagccgagaaggaagaaccctcaggaaggcctg
tacaatgaactgcagaaagataagatggcggaggcctacagtgagattgggatgaaaggcgagcgccggaggggcaagggg
cacgatggcctttaccagggtctcagtacagccaccaaggacacctacgacgcccttcacatgcaggccctgccccctcgc
tgataa
(amino acids)
(SEQ ID NO: 677)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRTESPKAQASSVPTAQPQAEGSLAKATTAPATTRNTGRGGEEKKKEKEKEEQEERETKTPAPELLGGPSVFLF
PPKPKDTLMISRTPEVTCVVVDVSHEDPEVKFNWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGFYPSDIAVEWESNGQPENNYKTTPPVLDSDGS
FFLRSKLTVDKSRWQQGNVFSCSVMHEALHNHYTQKSLSLSPGKIYIWAPLAGTCGVLLLSLVITLYCKRGRKKLLYIFK
QPFMRPVQTTQEEDGCSCRFPEEEEGGCELRVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDTYDALHMQALPPR**
IgD linker
(DNA)
(SEQ ID NO: 678)
gagtctccaaaggcacaggcctcctcagtgcccactgcacaaccccaagcagagggcagcctcgccaaggcaaccacagcc
ccagccaccacccgtaacacaggaagaggcggcgaagagaagaaaaaggagaaggagaaagaggaacaagaagagagagag
acaaagacacca
(amino acids)
(SEQ ID NO: 679)
ESPKAQASSVPTAQPQAEGSLAKATTAPATTRNTGRGGEEKKKEKEKEEQEERETKTP
C2 CAR IgD linker
(DNA)
(SEQ ID NO: 680)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaactgagtctccaaaggcacaggcctcctcagtgcccactgcacaaccccaagcagagggcagcctc
gccaaggcaaccacagccccagccaccacccgtaacacaggaagaggcggcgaagagaagaaaaaggagaaggagaaagag
gaacaagaagagagagagacaaagacaccaatctacatctgggcgcccttggccgggacttgtggggtccttctcctgtca
ctggttatcaccctttactgcaaacggggcagaaagaaactcctgtatatattcaaacaaccatttatgagaccagtacaa
actactcaagaggaagatggctgtagctgccgatttccagaagaagaagaaggaggatgtgaactgagagtgaagttcagc
aggagcgcagacgcccccgcgtacaagcagggccagaaccagctctataacgagctcaatctaggacgaagagaggagtac
gatgttttggacaagagacgtggccgggaccctgagatggggggaaagccgagaaggaagaaccctcaggaaggcctgtac
aatgaactgcagaaagataagatggcggaggcctacagtgagattgggatgaaaggcgagcgccggaggggcaaggggcac
gatggcctttaccagggtctcagtacagccaccaaggacacctacgacgcccttcacatgcaggccctgccccctcgctga
taa
(amino acids)
(SEQ ID NG: 681)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRTESPKAQASSVPTAQPQAEGSLAKATTAPATTRNTGRGGEEKKKEKEKEEQEERETKTPIYIWAPLAGTCGV
LLLSLVITLYCKRGRKKLLYIFKQPFMRPVQTTQEEDGCSCRFPEEEEGGCELRVKFSRSADAPAYKQGQNQLYNELNLO
RREEYDVLDKRRGRDPEMGGKPRRKNPQEGLYNELQKDKMAEAYSEIGMKGERRRGKGHDGLYQGLSTATKDTYDALHMQ
ALPPR**
X4 linker
(DNA)
(SEQ ID NO: 682)
gacaagacgcacaccaagccacctaaaccagctccagaactgctcggaggtcctggcaccggaaccggaggacctaccatc
aaaccacctaagccacctaagcctgctcctaacctgctcggaggacct
(amino acids)
(SEQ ID NO: 683)
DKTHTKPPKPAPELLGGPGTGTGGPTIKPPKPPKPAPNLLGGP
C2 CAR X4 linker
(DNA)
(SEQ ID NO: 684)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaactgacaagacgcacaccaagccacctaaaccagctccagaactgctcggaggtcctggcaccgga
accggaggacctaccatcaaaccacctaagccacctaagcctgctcctaacctgctcggaggacctatctacatctgggcg
cccttggccgggacttgtggggtccttctcctgtcactggttatcaccctttactgcaaacggggcagaaagaaactcctg
tatatattcaaacaaccatttatgagaccagtacaaactactcaagaggaagatggctgtagctgccgatttccagaagaa
gaagaaggaggatgtgaactgagagtgaagttcagcaggagcgcagacgcccccgcgtacaagcagggccagaaccagctc
tataacgagctcaatctaggacgaagagaggagtacgatgttttggacaagagacgtggccgggaccctgagatgggggga
aagccgagaaggaagaaccctcaggaaggcctgtacaatgaactgcagaaagataagatggcggaggcctacagtgagatt
gggatgaaaggcgagcgccggaggggcaaggggcacgatggcctttaccagggtctcagtacagccaccaaggacacctac
gacgcccttcacatgcaggccctgccccctcgctgataa
(amino acids)
(SEQ ID NO: 685)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRTDKTHTKPPKPAPELLGGPGTGTGGPTIKPPKPPKPAPNLLGGPIYIWAPLAGTCGVLLLSLVITLYCKRGR
KKLLYIFKQPFMRPVQTTQEEDGCSCRFPEEEEGGCELRVKFSRSADAPAYKQGQNQLYNELNLGRREEYDVLDKRRGRD
PEMGGKPRRKNPQEGLYNELQKDKMAEAYSEIGMKGERRRGKGHDGLYQGLSTATKDTYDALHMQALPPR**
OKT3 scFv
(DNA)
(SEQ ID NO: 686)
caggtgcagctggtgcagagcggaggcggagtggtgcagcctggaagaagcctgcgcctgagctgcaaagcgagcggctat
acctttacccgctataccatgcattgggtgcgccaggcgccgggcaaaggcctggaatggattggctatattaacccgagc
cgcggctataccaactataaccagaaagtgaaagatcgctttaccattagcaccgataaaagcaaaagcaccgcgtttctg
cagatggatagcctgcgcccggaagataccgcggtgtattattgcgcgcgctattatgatgatcattattgcctggattat
tggggccagggcaccaccctgaccgtgagcagcggcggtggcggatccggcggtggcggatccggcggtggcggatccgat
attcagatgacccagagcccgagcagcctgagcgcgagcgtgggcgatcgcgtgaccattacctgcagcgcgagcagcagc
gtgagctatatgaactggtatcagcagaccccgggcaaagcgccgaaacgctggatttatgataccagcaaactggcgagc
ggcgtgccgagccgctttagcggcagcggcagcggcaccgattatacctttaccattagcagcctgcagccggaagatatt
gcgacctattattgccagcagtggagcagcaacccgtttacctttggccagggcaccaaactgcagattacccgctgataa
(amino acids)
(SEQ ID NO: 687)
QVQLVQSGGGVVQPGRSLRLSCKASGYTFTRYTMHWVRQAPGKGLEWIGYINPSRGYTNYNQKVKDRFTISTDKSKSTAF
LQMDSLRPEDTAVYYCARYYDDHYCLDYWGQGTTLTVSSGGGGSGGGGSGGGGSDIQMTQSPSSLSASVGDRVTITCSAS
SSVSYMNWYQQTPGKAPKRWIYDTSKLASGVPSRFSGSGSGTDYTFTISSLQPEDIATYYCQQWSSNPFTFGQGTKLQIT
R**
C2-FC-OKT3
(DNA)
(SEQ ID NO: 688)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaactgagcccaaatcttgtgacaaaactcacacatgcccaccgtgcccagcacctgaactcctgggg
ggaccgtcagtcttcctcttccccccaaaacccaaggacaccctcatgatctcccggacccctgaggtcacatgcgtggtg
gtggacgtgagccacgaagaccctgaggtcaagttcaactggtacgtggacggcgtggaggtgcataatgccaagacaaag
ccgcgggaggagcagtacaacagcacgtaccgtgtggtcagcgtcctcaccgtcctgcaccaggactggctgaatggcaag
gagtacaagtgcaaggtctccaacaaagccctcccagcccccatcgagaaaaccatctccaaagccaaagggcagccccga
gaaccacaggtgtacaccctgcccccatcccgggaggagatgaccaagaaccaggtcagcctgacctgcctggtcaaaggc
ttctatcccagcgacatcgccgtggagtgggagagcaatgggcagccggagaacaactacaagaccacgcctcccgtgctg
gactccgacggctccttcttcctctacagcaagctcaccgtggacaagagcaggtggcagcaggggaacgtcttctcatgc
tccgtgatgcatgaggctctgcacaaccactacacgcagaagagcctctccctgtctccgggtaaaggcggtggcggatcc
caggtgcagctggtgcagagcggaggcggagtggtgcagcctggaagaagcctgcgcctgagctgcaaagcgagcggctat
acctttacccgctataccatgcattgggtgcgccaggcgccgggcaaaggcctggaatggattggctatattaacccgagc
cgcggctataccaactataaccagaaagtgaaagatcgctttaccattagcaccgataaaagcaaaagcaccgcgtttctg
cagatggatagcctgcgcccggaagataccgcggtgtattattgcgcgcgctattatgatgatcattattgcctggattat
tggggccagggcaccaccctgaccgtgagcagcggcggtggcggatccggcggtggcggatccggcggtggcggatccgat
attcagatgacccagagcccgagcagcctgagcgcgagcgtgggcgatcgcgtgaccattacctgcagcgcgagcagcagc
gtgagctatatgaactggtatcagcagaccccgggcaaagcgccgaaacgctggatttatgataccagcaaactggcgagc
ggcgtgccgagccgctttagcggcagcggcagcggcaccgattatacctttaccattagcagcctgcagccggaagatatt
gcgacctattattgccagcagtggagcagcaacccgtttacctttggccagggcaccaaactgcagattacccgctgataa
(amino acids)
(SEQ ID NO: 689)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRTEPKSCDKTHTCPPCPAPELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPREPQVYTLPPSREEMTKNQVSLT
CLVKGFYPSDIAVEWESNGQPENNYKTTPPVLDSDGSFFLYSKLTVDKSRWQQGNVFSCSVMHEALHNHYTQKSLSLSPG
KGGGGSQVQLVQSGGGVVQPGRSLRLSCKASGYTFTRYTMHWVRQAPGKGLEWIGYINPSRGYTNYNQKVKDRFTISTDK
SKSTAFLQMDSLRPEDTAVYYCARYYDDHYCLDYWGQGTTLTVSSGGGGSGGGGSGGGGSDIQMTQSPSSLSASVGDRVT
ITCSASSSVSYMNWYQQTPGKAPKRWIYDTSKLASGVPSRFSGSGSGTDYTFTISSLQPEDIATYYCQQWSSNPFTFGQG
TKLQITR**
C2-IgD/FC-OKT3
(DNA)
(SEQ ID NO: 690)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaactgagtctccaaaggcacaggcctcctcagtgcccactgcacaaccccaagcagagggcagcctc
gccaaggcaaccacagccccagccaccacccgtaacacaggaagaggcggcgaagagaagaaaaaggagaaggagaaagag
gaacaagaagagagagagacaaagacaccagagcccaaatcttgtgacaaaactcacacatgcccaccgtgcccagcacct
gaactcctggggggaccgtcagtcttcctcttccccccaaaacccaaggacaccctcatgatctcccggacccctgaggtc
acatgcgtggtggtggacgtgagccacgaagaccctgaggtcaagttcaactggtacgtggacggcgtggaggtgcataat
gccaagacaaagccgcgggaggagcagtacaacagcacgtaccgtgtggtcagcgtcctcaccgtcctgcaccaggactgg
ctgaatggcaaggagtacaagtgcaaggtctccaacaaagccctcccagcccccatcgagaaaaccatctccaaagccaaa
gggcagccccgagaaccacaggtgtacaccctgcccccatcccgggaggagatgaccaagaaccaggtcagcctgacctgc
ctggtcaaaggcttctatcccagcgacatcgccgtggagtgggagagcaatgggcagccggagaacaactacaagaccacg
cctcccgtgctggactccgacggctccttcttcctctacagcaagctcaccgtggacaagagcaggtggcagcaggggaac
gtcttctcatgctccgtgatgcatgaggctctgcacaaccactacacgcagaagagcctctccctgtctccgggtaaaggc
ggtggcggatcccaggtgcagctggtgcagagcggaggcggagtggtgcagcctggaagaagcctgcgcctgagctgcaaa
gcgagcggctatacctttacccgctataccatgcattgggtgcgccaggcgccgggcaaaggcctggaatggattggctat
attaacccgagccgcggctataccaactataaccagaaagtgaaagatcgctttaccattagcaccgataaaagcaaaagc
accgcgtttctgcagatggatagcctgcgcccggaagataccgcggtgtattattgcgcgcgctattatgatgatcattat
tgcctggattattggggccagggcaccaccctgaccgtgagcagcggcggtggcggatccggcggtggcggatccggcggt
ggcggatccgatattcagatgacccagagcccgagcagcctgagcgcgagcgtgggcgatcgcgtgaccattacctgcagc
gcgagcagcagcgtgagctatatgaactggtatcagcagaccccgggcaaagcgccgaaacgctggatttatgataccagc
aaactggcgagcggcgtgccgagccgctttagcggcagcggcagcggcaccgattatacctttaccattagcagcctgcag
ccggaagatattgcgacctattattgccagcagtggagcagcaacccgtttacctttggccagggcaccaaactgcagatt
acccgctgataa
(amino acids)
(SEQ ID NO: 691)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRTESPKAQASSVPTAQPQAEGSLAKATTAPATTRNTGRGGEEKKKEKEKEEQEERETKTPEPKSCDKTHTCPP
CPAPELLGGPSVFLFPPKPKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPGKGGGGSQVQLVQSGGGVVQPGR
SLRLSCKASGYTFTRYTMHWVRQAPGKGLEWIGYINPSRGYTNYNQKVKDRFTISTDKSKSTAFLQMDSLRPEDTAVYYC
ARYYDDHYCLDYWGQGTTLTVSSGGGGSGGGGSGGGGSDIQMTQSPSSLSASVGDRVTITCSASSSVSYMNWYQQTPGKA
PKRWIYDTSKLASGVPSRFSGSGSGTDYTFTISSLQPEDIATYYCQQWSSNPFTFGQGTKLQITR**
C2-FCHingeless/Y407R-OKT3
(DNA)
(SEQ ID NO: 692)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaactgcacctgaactcctggggggaccgtcagtcttcctcttccccccaaaacccaaggacaccctc
atgatctcccggacccctgaggtcacatgcgtggtggtggacgtgagccacgaagaccctgaggtcaagttcaactggtac
gtggacggcgtggaggtgcataatgccaagacaaagccgcgggaggagcagtacaacagcacgtaccgtgtggtcagcgtc
ctcaccgtcctgcaccaggactggctgaatggcaaggagtacaagtgcaaggtctccaacaaagccctcccagcccccatc
gagaaaaccatctccaaagccaaagggcagccccgagaaccacaggtgtacaccctgcccccatcccgggaggagatgacc
aagaaccaggtcagcctgacctgcctggtcaaaggcttctatcccagcgacatcgccgtggagtgggagagcaatgggcag
ccggagaacaactacaagaccacgcctcccgtgctggactccgacggctccttcttcctcaggagcaagctcaccgtggac
aagagcaggtggcagcaggggaacgtcttctcatgctccgtgatgcatgaggctctgcacaaccactacacgcagaagagc
ctctccctgtctccgggtaaaggcggtggcggatcccaggtgcagctggtgcagagcggaggcggagtggtgcagcctgga
agaagcctgcgcctgagctgcaaagcgagcggctatacctttacccgctataccatgcattgggtgcgccaggcgccgggc
aaaggcctggaatggattggctatattaacccgagccgcggctataccaactataaccagaaagtgaaagatcgctttacc
attagcaccgataaaagcaaaagcaccgcgtttctgcagatggatagcctgcgcccggaagataccgcggtgtattattgc
gcgcgctattatgatgatcattattgcctggattattggggccagggcaccaccctgaccgtgagcagcggcggtggcgga
tccggcggtggcggatccggcggtggcggatccgatattcagatgacccagagcccgagcagcctgagcgcgagcgtgggc
gatcgcgtgaccattacctgcagcgcgagcagcagcgtgagctatatgaactggtatcagcagaccccgggcaaagcgccg
aaacgctggatttatgataccagcaaactggcgagcggcgtgccgagccgctttagcggcagcggcagcggcaccgattat
acctttaccattagcagcctgcagccggaagatattgcgacctattattgccagcagtggagcagcaacccgtttaccttt
ggccagggcaccaaactgcagattacccgctgataa
(amino acids)
(SEQ ID NO: 693)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRTAPELLGGPSVFLFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSREEMTKNQVSLTCLVKGFYPSDIAVEW
ESNGQPENNYKTTPPVLDSDGSFFLRSKLTVDKSRWQQGNVFSCSVMHEALHNHYTQKSLSLSPGKGGGGSQVQLVQSGG
GVVQPGRSLRLSCKASGYTFTRYTMHWVRQAPGKGLEWIGYINPSRGYTNYNQKVKDRFTISTDKSKSTAFLQMDSLRPE
DTAVYYCARYYDDHYCLDYWGQGTTLTVSSGGGGSGGGGSGGGGSDIQMTQSPSSLSASVGDRVTITCSASSSVSYMNWY
QQTPGKAPKRWIYDTSKLASGVPSRFSGSGSGTDYTFTISSLQPEDIATYYCQQWSSNPFTFGQGTKLQITR**
C2-IgD/FCHingeless/Y407R-OKT3
(DNA)
(SEQ ID NO: 694)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaactgagtctccaaaggcacaggcctcctcagtgcccactgcacaaccccaagcagagggcagcctc
gccaaggcaaccacagccccagccaccacccgtaacacaggaagaggcggcgaagagaagaaaaaggagaaggagaaagag
gaacaagaagagagagagacaaagacaccagcacctgaactcctggggggaccgtcagtcttcctcttccccccaaaaccc
aaggacaccctcatgatctcccggacccctgaggtcacatgcgtggtggtggacgtgagccacgaagaccctgaggtcaag
ttcaactggtacgtggacggcgtggaggtgcataatgccaagacaaagccgcgggaggagcagtacaacagcacgtaccgt
gtggtcagcgtcctcaccgtcctgcaccaggactggctgaatggcaaggagtacaagtgcaaggtctccaacaaagccctc
ccagcccccatcgagaaaaccatctccaaagccaaagggcagccccgagaaccacaggtgtacaccctgcccccatcccgg
gaggagatgaccaagaaccaggtcagcctgacctgcctggtcaaaggcttctatcccagcgacatcgccgtggagtgggag
agcaatgggcagccggagaacaactacaagaccacgcctcccgtgctggactccgacggctccttcttcctcaggagcaag
ctcaccgtggacaagagcaggtggcagcaggggaacgtcttctcatgctccgtgatgcatgaggctctgcacaaccactac
acgcagaagagcctctccctgtctccgggtaaaggcggtggcggatcccaggtgcagctggtgcagagcggaggcggagtg
gtgcagcctggaagaagcctgcgcctgagctgcaaagcgagcggctatacctttacccgctataccatgcattgggtgcgc
caggcgccgggcaaaggcctggaatggattggctatattaacccgagccgcggctataccaactataaccagaaagtgaaa
gatcgctttaccattagcaccgataaaagcaaaagcaccgcgtttctgcagatggatagcctgcgcccggaagataccgcg
gtgtattattgcgcgcgctattatgatgatcattattgcctggattattggggccagggcaccaccctgaccgtgagcagc
ggcggtggcggatccggcggtggcggatccggcggtggcggatccgatattcagatgacccagagcccgagcagcctgagc
gcgagcgtgggcgatcgcgtgaccattacctgcagcgcgagcagcagcgtgagctatatgaactggtatcagcagaccccg
ggcaaagcgccgaaacgctggatttatgataccagcaaactggcgagcggcgtgccgagccgctttagcggcagcggcagc
ggcaccgattatacctttaccattagcagcctgcagccggaagatattgcgacctattattgccagcagtggagcagcaac
ccgtttacctttggccagggcaccaaactgcagattacccgctgataa
(amino acids)
(SEQ ID NO: 695)
EVQLVESGGGLVKPGGSLRLSCAASGFTFSGYAMSWVRQAPGKGLEWVSTISSGGTYIYYPDSVKGRFTISRDNAKNSLYL
QMNSLRAEDTAVYYCARLGGDNYYEYFDVWGKGTTVTVSSGGGGSGGGGSGGGGSDIVLTQSPASLAVSPGQRATITCRAS
KSVSTSGYSYMHWYQQKPGQPPKLLIYLASNLESGVPARFSGSGSGTDFTLTINPVEANDTANYYCQHSRELPFTFGGGTK
VEIKRTESPKAQASSVPTAQPQAEGSLAKATTAPATTRNTGRGGEEKKKEKEKEEQEERETKTPAPELLGGPSVFLFPPKP
KDTLMISRTPEVTCVVVDVSHEDPEVKFNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNGQPENNYKTTPPVLDSDGSFFLRSK
LTVDKSRWQQGNVFSCSVMHEALHNHYTQKSLSLSPGKGGGGSQVQLVQSGGGVVQPGRSLRLSCKASGYTFTRYTMHWVR
QAPGKGLEWIGYINPSRGYTNYNQKVKDRFTISTDKSKSTAFLQMDSLRPEDTAVYYCARYYDDHYCLDYWGQGTTLTVSS
GGGGSGGGGSGGGGSDIQMTQSPSSLSASVGDRVTITCSASSSVSYMNWYQQTPGKAPKRWIYDTSKLASGVPSRFSGSGS
GTDYTFTISSLQPEDIATYYCQQWSSNPFTFGQGTKLQITR**
C2-IgD-OKT3
(DNA)
(SEQ ID NO: 696)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaactgagtctccaaaggcacaggcctcctcagtgcccactgcacaaccccaagcagagggcagcctc
gccaaggcaaccacagccccagccaccacccgtaacacaggaagaggcggcgaagagaagaaaaaggagaaggagaaagag
gaacaagaagagagagagacaaagacaccaggcggtggcggatcccaggtgcagctggtgcagagcggaggcggagtggtg
cagcctggaagaagcctgcgcctgagctgcaaagcgagcggctatacctttacccgctataccatgcattgggtgcgccag
gcgccgggcaaaggcctggaatggattggctatattaacccgagccgcggctataccaactataaccagaaagtgaaagat
cgctttaccattagcaccgataaaagcaaaagcaccgcgtttctgcagatggatagcctgcgcccggaagataccgcggtg
tattattgcgcgcgctattatgatgatcattattgcctggattattggggccagggcaccaccctgaccgtgagcagcggc
ggtggcggatccggcggtggcggatccggcggtggcggatccgatattcagatgacccagagcccgagcagcctgagcgcg
agcgtgggcgatcgcgtgaccattacctgcagcgcgagcagcagcgtgagctatatgaactggtatcagcagaccccgggc
aaagcgccgaaacgctggatttatgataccagcaaactggcgagcggcgtgccgagccgctttagcggcagcggcagcggc
accgattatacctttaccattagcagcctgcagccggaagatattgcgacctattattgccagcagtggagcagcaacccg
tttacctttggccagggcaccaaactgcagattacccgctgataa
(amino acids)
(SEQ ID NO: 697)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRTESPKAQASSVPTAQPQAEGSLAKATTAPATTRNTGRGGEEKKKEKEKEEQEERETKTPGGGGSQVQLVQSG
GGVVQPGRSLRLSCKASGYTFTRYTMHWVRQAPGKGLEWIGYINPSRGYTNYNQKVKDRFTISTDKSKSTAFLQMDSLRP
EDTAVYYCARYYDDHYCLDYWGQGTTLTVSSGGGGSGGGGSGGGGSDIQMTQSPSSLSASVGDRVTITCSASSSVSYMNW
YQQTPGKAPKRWIYDTSKLASGVPSRFSGSGSGTDYTFTISSLQPEDIATYYCQQWSSNPFTFGQGTKLQITR**
C2-X4-OKT3
(DNA)
(SEQ ID NO: 698)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaactgacaagacgcacaccaagccacctaaaccagctccagaactgctcggaggtcctggcaccgga
accggaggacctaccatcaaaccacctaagccacctaagcctgctcctaacctgctcggaggacctggcggtggcggatcc
caggtgcagctggtgcagagcggaggcggagtggtgcagcctggaagaagcctgcgcctgagctgcaaagcgagcggctat
acctttacccgctataccatgcattgggtgcgccaggcgccgggcaaaggcctggaatggattggctatattaacccgagc
cgcggctataccaactataaccagaaagtgaaagatcgctttaccattagcaccgataaaagcaaaagcaccgcgtttctg
cagatggatagcctgcgcccggaagataccgcggtgtattattgcgcgcgctattatgatgatcattattgcctggattat
tggggccagggcaccaccctgaccgtgagcagcggcggtggcggatccggcggtggcggatccggcggtggcggatccgat
attcagatgacccagagcccgagcagcctgagcgcgagcgtgggcgatcgcgtgaccattacctgcagcgcgagcagcagc
gtgagctatatgaactggtatcagcagaccccgggcaaagcgccgaaacgctggatttatgataccagcaaactggcgagc
ggcgtgccgagccgctttagcggcagcggcagcggcaccgattatacctttaccattagcagcctgcagccggaagatatt
gcgacctattattgccagcagtggagcagcaacccgtttacctttggccagggcaccaaactgcagattacccgctgataa
(amino acids)
(SEQ ID NO: 699)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRTDKTHTKPPKPAPELLGGPGTGTGGPTIKPPKPPKPAPNLLGGPGGGGSQVQLVQSGGGVVQPGRSLRLSCK
ASGYTFTRYTMHWVRQAPGKGLEWIGYINPSRGYTNYNQKVKDRFTISTDKSKSTAFLQMDSLRPEDTAVYYCARYYDDH
YCLDYWGQGTTLTVSSGGGGSGGGGSGGGGSDIQMTQSPSSLSASVGDRVTITCSASSSVSYMNWYQQTPGKAPKRWIYD
TSKLASGVPSRFSGSGSGTDYTFTISSLQPEDIATYYCQQWSSNPFTFGQGTKLQITR**
C2-MMP9
(DNA)
(SEQ ID NO: 700)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaactggcggtggcggatccagcctctggcagcccctggtcctggtgctcctggtgctgggctgctgc
tttgctgcccccagacagcgccagtccacccttgtgctcttccctggagacctgagaaccaatctcaccgacaggcagctg
gcagaggaatacctgtaccgctatggttacactcgggtggcagagatgcgtggagagtcgaaatctctggggcctgcgctg
ctgcttctccagaagcaactgtccctgcccgagaccggtgagctggatagcgccacgctgaaggccatgcgaaccccacgg
tgcggggtcccagacctgggcagattccaaacctttgagggcgacctcaagtggcaccaccacaacatcacctattggatc
caaaactactcggaagacttgccgcgggcggtgattgacgacgcctttgcccgcgccttcgcactgtggagcgcggtgacg
ccgctcaccttcactcgcgtgtacagccgggacgcagacatcgtcatccagtttggtgtcgcggagcacggagacgggtat
cccttcgacgggaaggacgggctcctggcacacgcctttcctcctggccccggcattcagggagacgcccatttcgacgat
gacgagttgtggtccctgggcaagggcgtcgtggttccaactcggtttggaaacgcagatggcgcggcctgccacttcccc
ttcatcttcgagggccgctcctactctgcctgcaccaccgacggtcgctccgacggcttgccctggtgcagtaccacggcc
aactacgacaccgacgaccggtttggcttctgccccagcgagagactctacacccaggacggcaatgctgatgggaaaccc
tgccagtttccattcatcttccaaggccaatcctactccgcctgcaccacggacggtcgctccgacggctaccgctggtgc
gccaccaccgccaactacgaccgggacaagctcttcggcttctgcccgacccgagctgactcgacggtgatggggggcaac
tcggcgggggagctgtgcgtcttccccttcactttcctgggtaaggagtactcgacctgtaccagcgagggccgcggagat
gggcgcctctggtgcgctaccacctcgaactttgacagcgacaagaagtggggcttctgcccggaccaaggatacagtttg
ttcctcgtggcggcgcatgagttcggccacgcgctgggcttagatcattcctcagtgccggaggcgctcatgtaccctatg
taccgcttcactgaggggccccccttgcataaggacgacgtgaatggcatccggcacctctatggtcctcgccctgaacct
gagccacggcctccaaccaccaccacaccgcagcccacggctcccccgacggtctgccccaccggaccccccactgtccac
ccctcagagcgccccacagctggccccacaggtcccccctcagctggccccacaggtccccccactgctggcccttctacg
gccactactgtgcctttgagtccggtggacgatgcctgcaacgtgaacatcttcgacgccatcgcggagattgggaaccag
ctgtatttgttcaaggatgggaagtactggcgattctctgagggcagggggagccggccgcagggccccttccttatcgcc
gacaagtggcccgcgctgccccgcaagctggactcggtctttgaggagcggctctccaagaagcttttcttcttctctggg
cgccaggtgtgggtgtacacaggcgcgtcggtgctgggcccgaggcgtctggacaagctgggcctgggagccgacgtggcc
caggtgaccggggccctccggagtggcagggggaagatgctgctgttcagcgggcggcgcctctggaggttcgacgtgaag
gcgcagatggtggatccccggagcgccagcgaggtggaccggatgttccccggggtgcctttggacacgcacgacgtcttc
cagtaccgagagaaagcctatttctgccaggaccgcttctactggcgcgtgagttcccggagtgagttgaaccaggtggac
caagtgggctacgtgacctatgacatcctgcagtgccctgaggacgattacaaggatgacgacgataagtgataa
(amino acids)
(SEQ ID NO: 701)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRTGGGGSSLWQPLVLVLLVLGCCFAAPRQRQSTLVLFPGDLRTNLTDRQLAEEYLYRYGYTRVAEMRGESKSL
GPALLLLQKQLSLPETGELDSATLKAMRTPRCGVPDLGRFQTFEGDLKWHHHNITYWIQNYSEDLPRAVIDDAFARAFAL
WSAVTPLTFTRVYSRDADIVIQFGVAEHGDGYPFDGKDGLLAHAFPPGPGIQGDAHFDDDELWSLGKGVVVPTRFGNADG
AACHFPFIFEGRSYSACTTDGRSDGLPWCSTTANYDTDDRFGFCPSERLYTQDGNADGKPCQFPFIFQGQSYSACTTDGR
SDGYRWCATTANYDRDKLFGFCPTRADSTVMGGNSAGELCVFPFTFLGKEYSTCTSEGRGDGRLWCATTSNFDSDKKWGF
CPDQGYSLFLVAAHEFGHALGLDHSSVPEALMYPMYRFTEGPPLHKDDVNGIRHLYGPRPEPEPRPPTTTTPQPTAPPTV
CPTGPPTVHPSERPTAGPTGPPSAGPTGPPTAGPSTATTVPLSPVDDACNVNIFDAIAEIGNQLYLFKDGKYWRFSEGRG
SRPQGPFLIADKWPALPRKLDSVFEERLSKKLFFFSGRQVWVYTGASVLGPRRLDKLGLGADVAQVTGALRSGRGKMLLF
SGRRLWRFDVKAQMVDPRSASEVDRMFPGVPLDTHDVFQYREKAYFCQDRFYWRVSSRSELNQVDQVGYVTYDILQCPED
DYKDDDDK**
C2-MMP9cat
(DNA)
(SEQ ID NO: 702)
gaggtgcagctggtggagtctgggggaggcctggtcaagcctggggggtccctgagactctcctgtgcagcctctggattc
accttcagtggctatgccatgagctgggtccgccaggctccagggaaggggctggagtgggtctcaaccattagtagtggc
ggaacctacatatactaccccgactcagtgaagggccgattcaccatctccagagacaacgccaagaactcactgtatctg
caaatgaacagcctgagagccgaggacacggccgtgtattactgtgcgagacttgggggggataattactacgaatacttc
gatgtctggggcaaagggaccacggtcaccgtctcctccggcggtggcggatccggcggtggcggatccggcggtggcgga
tccgacattgtgctgacccagtctccagcctccttggccgtgtctccaggacagagggccaccatcacctgcagagccagt
aagagtgtcagtaccagcggatactcctacatgcactggtatcagcagaaaccaggacaacctcctaaactcctgatttac
ctggcatccaatctggagagcggggtcccagccaggttcagcggcagtgggtctgggaccgatttcaccctcacaattaat
cctgtggaagctaatgatactgcaaattattactgtcagcacagtagggagctgcctttcacattcggcggagggaccaag
gtggagatcaaacgaactggcggtggcggatccttccaaacctttgagggcgacctcaagtggcaccaccacaacatcacc
tattggatccaaaactactcggaagacttgccgcgggcggtgattgacgacgcctttgcccgcgccttcgcactgtggagc
gcggtgacgccgctcaccttcactcgcgtgtacagccgggacgcagacatcgtcatccagtttggtgtcgcggagcacgga
gacgggtatcccttcgacgggaaggacgggctcctggcacacgcctttcctcctggccccggcattcagggagacgcccat
ttcgacgatgacgagttgtggtccctgggcaagggcgtcgtggttccaactcggtttggaaacgcagatggcgcggcctgc
cacttccccttcatcttcgagggccgctcctactctgcctgcaccaccgacggtcgctccgacggcttgccctggtgcagt
accacggccaactacgacaccgacgaccggtttggcttctgccccagcgagagactctacacccaggacggcaatgctgat
gggaaaccctgccagtttccattcatcttccaaggccaatcctactccgcctgcaccacggacggtcgctccgacggctac
cgctggtgcgccaccaccgccaactacgaccgggacaagctcttcggcttctgcccgacccgagctgactcgacggtgatg
gggggcaactcggcgggggagctgtgcgtcttccccttcactttcctgggtaaggagtactcgacctgtaccagcgagggc
cgcggagatgggcgcctctggtgcgctaccacctcgaactttgacagcgacaagaagtggggcttctgcccggaccaagga
tacagtttgttcctcgtggcggcgcatgagttcggccacgcgctgggcttagatcattcctcagtgccggaggcgctcatg
taccctatgtaccgcttcactgaggggccccccttgcataaggacgacgtgaatggcatccggcacctctatggtcctcgc
cctgaacctgattacaaggatgacgacgataagtgataa
(amino acids)
(SEQ ID NO: 703)
EVQLVESGGGLVKPGGSLRLSCAASGFTFSGYAMSWVRQAPGKGLEWVSTISSGGTYIYYPDSVKGRFTISRDNAKNSLY
LQMNSLRAEDTAVYYCARLGGDNYYEYFDVWGKGTTVTVSSGGGGSGGGGSGGGGSDIVLTQSPASLAVSPGQRATITCR
ASKSVSTSGYSYMHWYQQKPGQPPKLLIYLASNLESGVPARFSGSGSGTDFTLTINPVEANDTANYYCQHSRELPFTFGG
GTKVEIKRTGGGGSFQTFEGDLKWHHHNITYWIQNYSEDLPRAVIDDAFARAFALWSAVTPLTFTRVYSRDADIVIQFGV
AEHGDGYPFDGKDGLLAHAFPPGPGIQGDAHFDDDELWSLGKGVVVPTRFGNADGAACHFPFIFEGRSYSACTTDGRSDG
LPWCSTTANYDTDDRFGFCPSERLYTQDGNADGKPCQFPFIFQGQSYSACTTDGRSDGYRWCATTANYDRDKLFGFCPTR
ADSTVMGGNSAGELCVFPFTFLGKEYSTCTSEGRGDGRLWCATTSNFDSDKKWGFCPDQGYSLFLVAAHEFGHALGLDHS
SVPEALMYPMYRFTEGPPLHKDDVNGIRHLYGPRPEPDYK
DDDDK**
Other linkers for BiTES between two scFvs and between C2 and MMP9 include but
are not limited to those shown as SEQ ID NOS: 705, 707, 709, 711, 713, 715 and
717.
[G₄S₁]x2 linker sequence:
(DNA)
(SEQ ID NO: 704)
ggcggtggcggatccggcggtggcggatcc
(amino acids)
(SEQ ID NO: 705)
GGGGSGGGGS
[G₄S₁]x3 linker sequence:
(DNA)
(SEQ ID NO: 706)
ggcggtggcggatccggcggtggcggatccggcggtggcggatcc
(amino acids)
(SEQ ID NO: 707)
GGGGSGGGGSGGGGS
Long GS linker sequence:
(DNA)
(SEQ ID NO: 708)
ggcggtggaagcggcggtggcggatccggcagcggcggaagcggcggtggcggatccggcggtgga
(amino acids)
(SEQ ID NO: 709)
GGGSGGGGSGSGGSGGGGSGGG
13 aa GS linker sequence:
(DNA)
(SEQ ID NO: 710)
ggcggtggatccggcggtggcggatccggcggtggatcc
(amino acids)
(SEQ ID NO: 711)
GGGSGGGGSGGGS
8 aa GS linker sequence:
(DNA)
(SEQ ID NO: 712)
ggcggttccggcggtggatccgga
(amino acids)
(SEQ ID NO: 713)
GGSGGGSG
12 aa GS linker sequence:
(DNA)
(SEQ ID NO: 714)
ggcggttccggcggtggatccggcggtggcggatccgga
(amino acids)
(SEQ ID NO: 715)
GGSGGGSGGGSG
24 aa GS linker sequence:
(DNA)
(SEQ ID NO: 716)
ggcggttccggcggtggatccggcggtggcggatccggaggcggttccggcggtggatccggcggtggcggatccgga
(amino acids)
(SEQ ID NO: 717)
GGSGGGSGGGSGGGSGGGSGGGSG
CAR-T C2 CD8/CD8/4-1BB/CD3z #44
N-CD8ls-huMNC2scFv-CD8ecd fragment- CD8 transmembrane- 4-1BB- CD3zeta-C
(DNA)
(SEQ ID NO: 718)
atggccttaccagtgaccgccttgctcctgccgctggccttgctgctccacgccgccaggccggaggtgcagctggtgga
gtctgggggaggcctggtcaagcctggggggtccctgagactctcctgtgcagcctctggattcaccttcagtggctatg
ccatgagctgggtccgccaggctccagggaaggggctggagtgggtctcaaccattagtagtggcggaacctacatatac
taccccgactcagtgaagggccgattcaccatctccagagacaacgccaagaactcactgtatctgcaaatgaacagcct
gagagccgaggacacggccgtgtattactgtgcgagacttgggggggataattactacgaatacttcgatgtctggggca
aagggaccacggtcaccgtctcctccggcggtggcggatccggcggtggcggatccggcggtggcggatccgacattgtg
ctgacccagtctccagcctccttggccgtgtctccaggacagagggccaccatcacctgcagagccagtaagagtgtcag
taccagcggatactcctacatgcactggtatcagcagaaaccaggacaacctcctaaactcctgatttacctggcatcca
atctggagagcggggtcccagccaggttcagcggcagtgggtctgggaccgatttcaccctcacaattaatcctgtggaa
gctaatgatactgcaaattattactgtcagcacagtagggagctgcctttcacattcggcggagggaccaaggtggagat
caaacgaactacaacaacccctgcccccagacctcctaccccagcccctacaattgccagccagcctctgagcctgaggc
ccgaggcttgtagacctgctgctggcggagccgtgcacaccagaggactggatttcgcctgcgacatctacatctgggcg
cccttggccgggacttgtggggtccttctcctgtcactggttatcaccctttactgcaaacggggcagaaagaaactcct
gtatatattcaaacaaccatttatgagaccagtacaaactactcaagaggaagatggctgtagctgccgatttccagaag
aagaagaaggaggatgtgaactgagagtgaagttcagcaggagcgcagacgcccccgcgtacaagcagggccagaaccag
ctctataacgagctcaatctaggacgaagagaggagtacgatgttttggacaagagacgtggccgggaccctgagatggg
gggaaagccgagaaggaagaaccctcaggaaggcctgtacaatgaactgcagaaagataagatggcggaggcctacagtg
agattgggatgaaaggcgagcgccggaggggcaaggggcacgatggcctttaccagggtctcagtacagccaccaaggac
acctacgacgcccttcacatgcaggccctgccccctcgctgataa
(amino acids)
(SEQ ID NO: 719)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTTTTPAPRPPTPAPTIASQPLSLRPEACRPAAGGAVHTRGLDFACDIYIWA
PLAGTCGVLLLSLVITLYCKRGRKKLLYIFKQPFMRPVQTTQEEDGCSCRFPEEEEGGCELRVKFSRSADAPAYKQGQNQ
LYNELNLGRREEYDVLDKRRGRDPEMGGKPRRKNPQEGLYNELQKDKMAEAYSEIGMKGERRRGKGHDGLYQGLSTATKD
TYDALHMQALPPR**
CAR-T IgK C2 CD8/CD8/4-1BB/CD3z #45
N-IgKls-huMNC2scFv-CD8ecd fragment- CD8 transmembrane- 4-1BB- CD3zeta-C
(DNA)
(SEQ ID NO: 720)
atggagacagacacactcctgctatgggtactgctgctctgggttccaggttccactggtgaggtgcagctggtggagtc
tgggggaggcctggtcaagcctggggggtccctgagactctcctgtgcagcctctggattcaccttcagtggctatgcca
tgagctgggtccgccaggctccagggaaggggctggagtgggtctcaaccattagtagtggcggaacctacatatactac
cccgactcagtgaagggccgattcaccatctccagagacaacgccaagaactcactgtatctgcaaatgaacagcctgag
agccgaggacacggccgtgtattactgtgcgagacttgggggggataattactacgaatacttcgatgtctggggcaaag
ggaccacggtcaccgtctcctccggcggtggcggatccggcggtggcggatccggcggtggcggatccgacattgtgctg
acccagtctccagcctccttggccgtgtctccaggacagagggccaccatcacctgcagagccagtaagagtgtcagtac
cagcggatactcctacatgcactggtatcagcagaaaccaggacaacctcctaaactcctgatttacctggcatccaatc
tggagagcggggtcccagccaggttcagcggcagtgggtctgggaccgatttcaccctcacaattaatcctgtggaagct
aatgatactgcaaattattactgtcagcacagtagggagctgcctttcacattcggcggagggaccaaggtggagatcaa
acgaactacaacaacccctgcccccagacctcctaccccagcccctacaattgccagccagcctctgagcctgaggcccg
aggcttgtagacctgctgctggcggagccgtgcacaccagaggactggatttcgcctgcgacatctacatctgggcgccc
ttggccgggacttgtggggtccttctcctgtcactggttatcaccctttactgcaaacggggcagaaagaaactcctgta
tatattcaaacaaccatttatgagaccagtacaaactactcaagaggaagatggctgtagctgccgatttccagaagaag
aagaaggaggatgtgaactgagagtgaagttcagcaggagcgcagacgcccccgcgtacaagcagggccagaaccagctc
tataacgagctcaatctaggacgaagagaggagtacgatgttttggacaagagacgtggccgggaccctgagatgggggg
aaagccgagaaggaagaaccctcaggaaggcctgtacaatgaactgcagaaagataagatggcggaggcctacagtgaga
ttgggatgaaaggcgagcgccggaggggcaaggggcacgatggcctttaccagggtctcagtacagccaccaaggacacc
tacgacgcccttcacatgcaggccctgccccctcgctgataa
(amino acids)
(SEQ ID NO: 721)
METDTLLLWVLLLWVPGSTGEVQLVESGGGLVKPGGSLRLSCAASGFTFSGYAMSWVRQAPGKGLEWVSTISSGGTYIYY
PDSVKGRFTISRDNAKNSLYLQMNSLRAEDTAVYYCARLGGDNYYEYFDVWGKGTTVTVSSGGGGSGGGGSGGGGSDIVL
TQSPASLAVSPGQRATITCRASKSVSTSGYSYMHWYQQKPGQPPKLLIYLASNLESGVPARFSGSGSGTDFTLTINPVEA
NDTANYYCQHSRELPFTFGGGTKVEIKRTTTTPAPRPPTPAPTIASQPLSLRPEACRPAAGGAVHTRGLDFACDIYIWAP
LAGTCGVLLLSLVITLYCKRGRKKLLYIFKQPFMRPVQTTQEEDGCSCRFPEEEEGGCELRVKFSRSADAPAYKQGQNQL
YNELNLGRREEYDVLDKRRGRDPEMGGKPRRKNPQEGLYNELQKDKMAEAYSEIGMKGERRRGKGHDGLYQGLSTATKDT
YDALHMQALPPR**
CAR-T C2 op CD8/CD8/4-1BB/CD3z #46
N-CD8ls-huMNC2scFv codon optimized-CD8ecd fragment- CD8 transmembrane- 4-1BB-
CD3zeta-C
(DNA)
(SEQ ID NO: 722)
atggccttaccagtgaccgccttgctcctgccgctggccttgctgctccacgccgccaggccggaagtgcagctggtgga
atctggcggcggactcgtgaagcctggcggctctctgagactgagctgtgccgccagcggcttcacctttagcggctacg
ccatgagctgggtgcgccaggctcctggcaaaggcctggaatgggtgtccaccatctctagcggcggcacctacatctac
taccccgacagcgtgaagggccggttcaccatcagccgggacaacgccaagaacagcctgtacctgcagatgaactccct
gcgggccgaggacaccgccgtgtactattgtgctagactgggcggcgacaactactacgagtacttcgacgtgtggggca
agggcaccaccgtgacagtgtctagcggaggcggaggatcaggcggcggaggaagtggcggagggggatctgatatcgtg
ctgacccagagccctgccagcctggctgtgtctcctggacagagggccaccatcacctgtcgggccagcaagagcgtgtc
cacctccggctacagctacatgcactggtatcagcagaagcccggccagccccccaagctgctgatctacctggccagca
acctggaaagcggcgtgcccgctagattttccggctctggcagcggcaccgacttcaccctgaccatcaaccccgtggaa
gccaacgacaccgccaattactactgccagcacagcagagagctgcccttcaccttcggcggaggcaccaaggtggaaat
caagcggaccacaacaacccctgcccccagacctcctaccccagcccctacaattgccagccagcctctgagcctgaggc
ccgaggcttgtagacctgctgctggcggagccgtgcacaccagaggactggatttcgcctgcgacatctacatctgggcg
cccttggccgggacttgtggggtccttctcctgtcactggttatcaccctttactgcaaacggggcagaaagaaactcct
gtatatattcaaacaaccatttatgagaccagtacaaactactcaagaggaagatggctgtagctgccgatttccagaag
aagaagaaggaggatgtgaactgagagtgaagttcagcaggagcgcagacgcccccgcgtacaagcagggccagaaccag
ctctataacgagctcaatctaggacgaagagaggagtacgatgttttggacaagagacgtggccgggaccctgagatggg
gggaaagccgagaaggaagaaccctcaggaaggcctgtacaatgaactgcagaaagataagatggcggaggcctacagtg
agattgggatgaaaggcgagcgccggaggggcaaggggcacgatggcctttaccagggtctcagtacagccaccaaggac
acctacgacgcccttcacatgcaggccctgccccctcgctgataa
(amino acids)
(SEQ ID NO: 723)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTTTTPAPRPPTPAPTIASQPLSLRPEACRPAAGGAVHTRGLDFACDIYIWA
PLAGTCGVLLLSLVITLYCKRGRKKLLYIFKQPFMRPVQTTQEEDGCSCRFPEEEEGGCELRVKFSRSADAPAYKQGQNQ
LYNELNLGRREEYDVLDKRRGRDPEMGGKPRRKNPQEGLYNELQKDKMAEAYSEIGMKGERRRGKGHDGLYQGLSTATKD
TYDALHMQALPPR**
CAR-T IgK C2 op CD8/CD8/4-1BB/CD3z #47
N-IgKls-huMNC2scFv codon optimized-CD8ecd fragment- CD8 transmembrane- 4-1BB-
CD3zeta-C
(DNA)
(SEQ ID NO: 724)
atggagacagacacactcctgctatgggtactgctgctctgggttccaggttccactggtgaagtgcagctggtggaatc
tggcggcggactcgtgaagcctggcggctctctgagactgagctgtgccgccagcggcttcacctttagcggctacgcca
tgagctgggtgcgccaggctcctggcaaaggcctggaatgggtgtccaccatctctagcggcggcacctacatctactac
cccgacagcgtgaagggccggttcaccatcagccgggacaacgccaagaacagcctgtacctgcagatgaactccctgcg
ggccgaggacaccgccgtgtactattgtgctagactgggcggcgacaactactacgagtacttcgacgtgtggggcaagg
gcaccaccgtgacagtgtctagcggaggcggaggatcaggcggcggaggaagtggcggagggggatctgatatcgtgctg
acccagagccctgccagcctggctgtgtctcctggacagagggccaccatcacctgtcgggccagcaagagcgtgtccac
ctccggctacagctacatgcactggtatcagcagaagcccggccagccccccaagctgctgatctacctggccagcaacc
tggaaagcggcgtgcccgctagattttccggctctggcagcggcaccgacttcaccctgaccatcaaccccgtggaagcc
aacgacaccgccaattactactgccagcacagcagagagctgcccttcaccttcggcggaggcaccaaggtggaaatcaa
gcggaccacaacaacccctgcccccagacctcctaccccagcccctacaattgccagccagcctctgagcctgaggcccg
aggcttgtagacctgctgctggcggagccgtgcacaccagaggactggatttcgcctgcgacatctacatctgggcgccc
ttggccgggacttgtggggtccttctcctgtcactggttatcaccctttactgcaaacggggcagaaagaaactcctgta
tatattcaaacaaccatttatgagaccagtacaaactactcaagaggaagatggctgtagctgccgatttccagaagaag
aagaaggaggatgtgaactgagagtgaagttcagcaggagcgcagacgcccccgcgtacaagcagggccagaaccagctc
tataacgagctcaatctaggacgaagagaggagtacgatgttttggacaagagacgtggccgggaccctgagatgggggg
aaagccgagaaggaagaaccctcaggaaggcctgtacaatgaactgcagaaagataagatggcggaggcctacagtgaga
ttgggatgaaaggcgagcgccggaggggcaaggggcacgatggcctttaccagggtctcagtacagccaccaaggacacc
tacgacgcccttcacatgcaggccctgccccctcgctgataa
(amino acids)
(SEQ ID NO: 725)
METDTLLLWVLLLWVPGSTGEVQLVESGGGLVKPGGSLRLSCAASGFTFSGYAMSWVRQAPGKGLEWVSTISSGGTYIYY
PDSVKGRFTISRDNAKNSLYLQMNSLRAEDTAVYYCARLGGDNYYEYFDVWGKGTTVTVSSGGGGSGGGGSGGGGSDIVL
TQSPASLAVSPGQRATITCRASKSVSTSGYSYMHWYQQKPGQPPKLLIYLASNLESGVPARFSGSGSGTDFTLTINPVEA
NDTANYYCQHSRELPFTFGGGTKVEIKRTTTTPAPRPPTPAPTIASQPLSLRPEACRPAAGGAVHTRGLDFACDIYIWAP
LAGTCGVLLLSLVITLYCKRGRKKLLYIFKQPFMRPVQTTQEEDGCSCRFPEEEEGGCELRVKFSRSADAPAYKQGQNQL
YNELNLGRREEYDVLDKRRGRDPEMGGKPRRKNPQEGLYNELQKDKMAEAYSEIGMKGERRRGKGHDGLYQGLSTATKDT
YDALHMQALPPR**
CAR-T C2 CD8/CD8/4-1BB/CD3z op #48
N-CD8ls-huMNC2scFv -CD8ecd fragment- CD8 transmembrane- 4-1BB- CD3zeta (all
domains codon optimized)-C
(DNA)
(SEQ ID NO: 726)
atggccttaccagtgaccgccttgctcctgccgctggccttgctgctccacgccgccaggccggaagtgcagctggtgga
atctggcggcggactcgtgaagcctggcggctctctgagactgagctgtgccgccagcggcttcacctttagcggctacg
ccatgagctgggtgcgccaggctcctggcaaaggcctggaatgggtgtccaccatctctagcggcggcacctacatctac
taccccgacagcgtgaagggccggttcaccatcagccgggacaacgccaagaacagcctgtacctgcagatgaactccct
gcgggccgaggacaccgccgtgtactattgtgctagactgggcggcgacaactactacgagtacttcgacgtgtggggca
agggcaccaccgtgacagtgtctagcggaggcggaggatcaggcggcggaggaagtggcggagggggatctgatatcgtg
ctgacccagagccctgccagcctggctgtgtctcctggacagagggccaccatcacctgtcgggccagcaagagcgtgtc
cacctccggctacagctacatgcactggtatcagcagaagcccggccagccccccaagctgctgatctacctggccagca
acctggaaagcggcgtgcccgctagattttccggctctggcagcggcaccgacttcaccctgaccatcaaccccgtggaa
gccaacgacaccgccaattactactgccagcacagcagagagctgcccttcaccttcggcggaggcaccaaggtggaaat
caagcggaccacaacaacccctgcccccagacctcctaccccagcccctacaattgccagccagcctctgagcctgaggc
ccgaggcttgtagacctgctgctggcggagccgtgcacaccagaggactggatttcgcctgcgacatctacatctgggcc
cctctggccggcacatgtggcgtgctgctgctgagcctcgtgatcaccctgtactgcaagcggggcagaaagaagctgct
gtacatcttcaagcagcccttcatgcggcccgtgcagaccacccaggaagaggacggctgctcctgcagattccccgagg
aagaagaaggcggctgcgagctgagagtgaagttcagcagatccgccgacgcccctgcctacaagcagggccagaaccag
ctgtacaacgagctgaacctgggcagacgggaagagtacgacgtgctggacaagcggagaggcagggaccctgagatggg
cggcaagcccagaagaaagaacccccaggaaggcctgtataacgaactgcagaaagacaagatggccgaggcctacagcg
agatcggaatgaagggcgagcggagaagaggcaagggccacgatggcctgtaccagggcctgagcaccgccaccaaggac
acctatgacgccctgcacatgcaggccctgcctcccagatgataa
(amino acids)
(SEQ ID NO: 727)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTTTTPAPRPPTPAPTIASQPLSLRPEACRPAAGGAVHTRGLDFACDIYIWA
PLAGTCGVLLLSLVITLYCKRGRKKLLYIFKQPFMRPVQTTQEEDGCSCRFPEEEEGGCELRVKFSRSADAPAYKQGQNQ
LYNELNLGRREEYDVLDKRRGRDPEMGGKPRRKNPQEGLYNELQKDKMAEAYSEIGMKGERRRGKGHDGLYQGLSTATKD
TYDALHMQALPPR**
CAR-T IgK C2 CD8/CD8/4-1BB/CD3z op #49
N-IgKls-huMNC2scFv -CD8ecd fragment- CD8 transmembrane- 4-1BB- CD3zeta (all
domains codon optimized)-C
(DNA)
(SEQ ID NO: 728)
atggagacagacacactcctgctatgggtactgctgctctgggttccaggttccactggtgaagtgcagctggtggaatc
tggcggcggactcgtgaagcctggcggctctctgagactgagctgtgccgccagcggcttcacctttagcggctacgcca
tgagctgggtgcgccaggctcctggcaaaggcctggaatgggtgtccaccatctctagcggcggcacctacatctactac
cccgacagcgtgaagggccggttcaccatcagccgggacaacgccaagaacagcctgtacctgcagatgaactccctgcg
ggccgaggacaccgccgtgtactattgtgctagactgggcggcgacaactactacgagtacttcgacgtgtggggcaagg
gcaccaccgtgacagtgtctagcggaggcggaggatcaggcggcggaggaagtggcggagggggatctgatatcgtgctg
acccagagccctgccagcctggctgtgtctcctggacagagggccaccatcacctgtcgggccagcaagagcgtgtccac
ctccggctacagctacatgcactggtatcagcagaagcccggccagccccccaagctgctgatctacctggccagcaacc
tggaaagcggcgtgcccgctagattttccggctctggcagcggcaccgacttcaccctgaccatcaaccccgtggaagcc
aacgacaccgccaattactactgccagcacagcagagagctgcccttcaccttcggcggaggcaccaaggtggaaatcaa
gcggaccacaacaacccctgcccccagacctcctaccccagcccctacaattgccagccagcctctgagcctgaggcccg
aggcttgtagacctgctgctggcggagccgtgcacaccagaggactggatttcgcctgcgacatctacatctgggcccct
ctggccggcacatgtggcgtgctgctgctgagcctcgtgatcaccctgtactgcaagcggggcagaaagaagctgctgta
catcttcaagcagcccttcatgcggcccgtgcagaccacccaggaagaggacggctgctcctgcagattccccgaggaag
aagaaggcggctgcgagctgagagtgaagttcagcagatccgccgacgcccctgcctacaagcagggccagaaccagctg
tacaacgagctgaacctgggcagacgggaagagtacgacgtgctggacaagcggagaggcagggaccctgagatgggcgg
caagcccagaagaaagaacccccaggaaggcctgtataacgaactgcagaaagacaagatggccgaggcctacagcgaga
tcggaatgaagggcgagcggagaagaggcaagggccacgatggcctgtaccagggcctgagcaccgccaccaaggacacc
tatgacgccctgcacatgcaggccctgcctcccagatgataa
(amino acids)
(SEQ ID NO: 729)
METDTLLLWVLLLWVPGSTGEVQLVESGGGLVKPGGSLRLSCAASGFTFSGYAMSWVRQAPGKGLEWVSTISSGGTYIYY
PDSVKGRFTISRDNAKNSLYLQMNSLRAEDTAVYYCARLGGDNYYEYFDVWGKGTTVTVSSGGGGSGGGGSGGGGSDIVL
TQSPASLAVSPGQRATITCRASKSVSTSGYSYMHWYQQKPGQPPKLLIYLASNLESGVPARFSGSGSGTDFTLTINPVEA
NDTANYYCQHSRELPFTFGGGTKVEIKRTTTTPAPRPPTPAPTIASQPLSLRPEACRPAAGGAVHTRGLDFACDIYIWAP
LAGTCGVLLLSLVITLYCKRGRKKLLYIFKQPFMRPVQTTQEEDGCSCRFPEEEEGGCELRVKFSRSADAPAYKQGQNQL
YNELNLGRREEYDVLDKRRGRDPEMGGKPRRKNPQEGLYNELQKDKMAEAYSEIGMKGERRRGKGHDGLYQGLSTATKDT
YDALHMQALPPR**
CAR-T C2 CD4/CD4/4-1BB/CD3z #50
N-CD8ls-huMNC2scFv-CD4ecd fragment- CD4 transmembrane- 4-1BB- CD3zeta-C
(DNA)
(SEQ ID NO: 730)
atggccttaccagtgaccgccttgctcctgccgctggccttgctgctccacgccgccaggccggaggtgcagctggtgga
gtctgggggaggcctggtcaagcctggggggtccctgagactctcctgtgcagcctctggattcaccttcagtggctatg
ccatgagctgggtccgccaggctccagggaaggggctggagtgggtctcaaccattagtagtggcggaacctacatatac
taccccgactcagtgaagggccgattcaccatctccagagacaacgccaagaactcactgtatctgcaaatgaacagcct
gagagccgaggacacggccgtgtattactgtgcgagacttgggggggataattactacgaatacttcgatgtctggggca
aagggaccacggtcaccgtctcctccggcggtggcggatccggcggtggcggatccggcggtggcggatccgacattgtg
ctgacccagtctccagcctccttggccgtgtctccaggacagagggccaccatcacctgcagagccagtaagagtgtcag
taccagcggatactcctacatgcactggtatcagcagaaaccaggacaacctcctaaactcctgatttacctggcatcca
atctggagagcggggtcccagccaggttcagcggcagtgggtctgggaccgatttcaccctcacaattaatcctgtggaa
gctaatgatactgcaaattattactgtcagcacagtagggagctgcctttcacattcggcggagggaccaaggtggagat
caaacgaacttcgggacaggtcctgctggaatccaacatcaaggttctgcccacatggtccaccccggtgcagccaatgg
ccctgattgtgctggggggcgtcgccggcctcctgcttttcattgggctaggcatcttcttcaaacggggcagaaagaaa
ctcctgtatatattcaaacaaccatttatgagaccagtacaaactactcaagaggaagatggctgtagctgccgatttcc
agaagaagaagaaggaggatgtgaactgagagtgaagttcagcaggagcgcagacgcccccgcgtacaagcagggccaga
accagctctataacgagctcaatctaggacgaagagaggagtacgatgttttggacaagagacgtggccgggaccctgag
atggggggaaagccgagaaggaagaaccctcaggaaggcctgtacaatgaactgcagaaagataagatggcggaggccta
cagtgagattgggatgaaaggcgagcgccggaggggcaaggggcacgatggcctttaccagggtctcagtacagccacca
aggacacctacgacgcccttcacatgcaggccctgccccctcgctgataa
(amino acids)
(SEQ ID NO: 731)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTSGQVLLESNIKVLPTWSTPVQPMALIVLGGVAGLLLFIGLGIFFKRGRKK
LLYIFKQPFMRPVQTTQEEDGCSCRFPEEEEGGCELRVKFSRSADAPAYKQGQNQLYNELNLGRREEYDVLDKRRGRDPE
MGGKPRRKNPQEGLYNELQKDKMAEAYSEIGMKGERRRGKGHDGLYQGLSTATKDTYDALHMQALPPR**
CAR-T C2 FC/CD8/4-1BB/CD3z “Fc” CAR53
N-CD8ls-huMNC2scFv-Human IgG1 Fc- CD8 transmembrane- 4-1BB- CD3zeta-C
(DNA)
(SEQ ID NO: 732)
atggccttaccagtgaccgccttgctcctgccgctggccttgctgctccacgccgccaggccggaggtgcagctggtgga
gtctgggggaggcctggtcaagcctggggggtccctgagactctcctgtgcagcctctggattcaccttcagtggctatg
ccatgagctgggtccgccaggctccagggaaggggctggagtgggtctcaaccattagtagtggcggaacctacatatac
taccccgactcagtgaagggccgattcaccatctccagagacaacgccaagaactcactgtatctgcaaatgaacagcct
gagagccgaggacacggccgtgtattactgtgcgagacttgggggggataattactacgaatacttcgatgtctggggca
aagggaccacggtcaccgtctcctccggcggtggcggatccggcggtggcggatccggcggtggcggatccgacattgtg
ctgacccagtctccagcctccttggccgtgtctccaggacagagggccaccatcacctgcagagccagtaagagtgtcag
taccagcggatactcctacatgcactggtatcagcagaaaccaggacaacctcctaaactcctgatttacctggcatcca
atctggagagcggggtcccagccaggttcagcggcagtgggtctgggaccgatttcaccctcacaattaatcctgtggaa
gctaatgatactgcaaattattactgtcagcacagtagggagctgcctttcacattcggcggagggaccaaggtggagat
caaacgaactgagcccaaatcttgtgacaaaactcacacatgcccaccgtgcccagcacctgaactcctggggggaccgt
cagtcttcctcttccccccaaaacccaaggacaccctcatgatctcccggacccctgaggtcacatgcgtggtggtggac
gtgagccacgaagaccctgaggtcaagttcaactggtacgtggacggcgtggaggtgcataatgccaagacaaagccgcg
ggaggagcagtacaacagcacgtaccgtgtggtcagcgtcctcaccgtcctgcaccaggactggctgaatggcaaggagt
acaagtgcaaggtctccaacaaagccctcccagcccccatcgagaaaaccatctccaaagccaaagggcagccccgagaa
ccacaggtgtacaccctgcccccatcccgggaggagatgaccaagaaccaggtcagcctgacctgcctggtcaaaggctt
ctatcccagcgacatcgccgtggagtgggagagcaatgggcagccggagaacaactacaagaccacgcctcccgtgctgg
actccgacggctccttcttcctctacagcaagctcaccgtggacaagagcaggtggcagcaggggaacgtcttctcatgc
tccgtgatgcatgaggctctgcacaaccactacacgcagaagagcctctccctgtctccgggtaaaatctacatctgggc
gcccttggccgggacttgtggggtccttctcctgtcactggttatcaccctttactgcaaacggggcagaaagaaactcc
tgtatatattcaaacaaccatttatgagaccagtacaaactactcaagaggaagatggctgtagctgccgatttccagaa
gaagaagaaggaggatgtgaactgagagtgaagttcagcaggagcgcagacgcccccgcgtacaagcagggccagaacca
gctctataacgagctcaatctaggacgaagagaggagtacgatgttttggacaagagacgtggccgggaccctgagatgg
ggggaaagccgagaaggaagaaccctcaggaaggcctgtacaatgaactgcagaaagataagatggcggaggcctacagt
gagattgggatgaaaggcgagcgccggaggggcaaggggcacgatggcctttaccagggtctcagtacagccaccaagga
cacctacgacgcccttcacatgcaggccctgccccctcgctgataa
(amino acids)
(SEQ ID NO: 733)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTEPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTPPVLDSDGSFFLYSKLTVDKSRWQQGNVFSC
SVMHEALHNHYTQKSLSLSPGKIYIWAPLAGTCGVLLLSLVITLYCKRGRKKLLYIFKQPFMRPVQTTQEEDGCSCRFPE
EEEGGCELRVKFSRSADAPAYKQGQNQLYNELNLGRREEYDVLDKRRGRDPEMGGKPRRKNPQEGLYNELQKDKMAEAYS
EIGMKGERRRGKGHDGLYQGLSTATKDTYDALHMQALPPR**
CAR-T C2 IgD/FC/CD8/4-1BB/CD3z “IgD-Fc” CAR54
N-CD8ls-huMNC2scFv- IgD hinge- Human IgG1 Fc- CD8 transmembrane- 4-1BB-
CD3zeta-C
(DNA)
(SEQ ID NO: 734)
atggccttaccagtgaccgccttgctcctgccgctggccttgctgctccacgccgccaggccggaggtgcagctggtgga
gtctgggggaggcctggtcaagcctggggggtccctgagactctcctgtgcagcctctggattcaccttcagtggctatg
ccatgagctgggtccgccaggctccagggaaggggctggagtgggtctcaaccattagtagtggcggaacctacatatac
taccccgactcagtgaagggccgattcaccatctccagagacaacgccaagaactcactgtatctgcaaatgaacagcct
gagagccgaggacacggccgtgtattactgtgcgagacttgggggggataattactacgaatacttcgatgtctggggca
aagggaccacggtcaccgtctcctccggcggtggcggatccggcggtggcggatccggcggtggcggatccgacattgtg
ctgacccagtctccagcctccttggccgtgtctccaggacagagggccaccatcacctgcagagccagtaagagtgtcag
taccagcggatactcctacatgcactggtatcagcagaaaccaggacaacctcctaaactcctgatttacctggcatcca
atctggagagcggggtcccagccaggttcagcggcagtgggtctgggaccgatttcaccctcacaattaatcctgtggaa
gctaatgatactgcaaattattactgtcagcacagtagggagctgcctttcacattcggcggagggaccaaggtggagat
caaacgaactgagtctccaaaggcacaggcctcctcagtgcccactgcacaaccccaagcagagggcagcctcgccaagg
caaccacagccccagccaccacccgtaacacaggaagaggcggcgaagagaagaaaaaggagaaggagaaagaggaacaa
gaagagagagagacaaagacaccagagcccaaatcttgtgacaaaactcacacatgcccaccgtgcccagcacctgaact
cctggggggaccgtcagtcttcctcttccccccaaaacccaaggacaccctcatgatctcccggacccctgaggtcacat
gcgtggtggtggacgtgagccacgaagaccctgaggtcaagttcaactggtacgtggacggcgtggaggtgcataatgcc
aagacaaagccgcgggaggagcagtacaacagcacgtaccgtgtggtcagcgtcctcaccgtcctgcaccaggactggct
gaatggcaaggagtacaagtgcaaggtctccaacaaagccctcccagcccccatcgagaaaaccatctccaaagccaaag
ggcagccccgagaaccacaggtgtacaccctgcccccatcccgggaggagatgaccaagaaccaggtcagcctgacctgc
ctggtcaaaggcttctatcccagcgacatcgccgtggagtgggagagcaatgggcagccggagaacaactacaagaccac
gcctcccgtgctggactccgacggctccttcttcctctacagcaagctcaccgtggacaagagcaggtggcagcagggga
acgtcttctcatgctccgtgatgcatgaggctctgcacaaccactacacgcagaagagcctctccctgtctccgggtaaa
atctacatctgggcgcccttggccgggacttgtggggtccttctcctgtcactggttatcaccctttactgcaaacgggg
cagaaagaaactcctgtatatattcaaacaaccatttatgagaccagtacaaactactcaagaggaagatggctgtagct
gccgatttccagaagaagaagaaggaggatgtgaactgagagtgaagttcagcaggagcgcagacgcccccgcgtacaag
cagggccagaaccagctctataacgagctcaatctaggacgaagagaggagtacgatgttttggacaagagacgtggccg
ggaccctgagatggggggaaagccgagaaggaagaaccctcaggaaggcctgtacaatgaactgcagaaagataagatgg
cggaggcctacagtgagattgggatgaaaggcgagcgccggaggggcaaggggcacgatggcctttaccagggtctcagt
acagccaccaaggacacctacgacgcccttcacatgcaggccctgccccctcgctgataa
(amino acids)
(SEQ ID NO: 735)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTESPKAQASSVPTAQPQAEGSLAKATTAPATTRNTGRGGEEKKKEKEKEEQ
EERETKTPEPKSCDKTHTCPPCPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRWQQGNVFSCSVMHEALHNHYTQKSLSLSPGK
IYIWAPLAGTCGVLLLSLVITLYCKRGRKKLLYIFKQPFMRPVQTTQEEDGCSCRFPEEEEGGCELRVKFSRSADAPAYK
QGQNQLYNELNLGRREEYDVLDKRRGRDPEMGGKPRRKNPQEGLYNELQKDKMAEAYSEIGMKGERRRGKGHDGLYQGLS
TATKDTYDALHMQALPPR**
CAR-T C2 FCHingeless Y407R/CD8/4-1BB/CD3z “FcH” CAR55
N-CD8ls-huMNC2scFv-Human IgG1 hingeless Fc Y407R- CD8 transmembrane- 4-1BB-
CD3zeta-C
(DNA)
(SEQ ID NO: 736)
atggccttaccagtgaccgccttgctcctgccgctggccttgctgctccacgccgccaggccggaggtgcagctggtgga
gtctgggggaggcctggtcaagcctggggggtccctgagactctcctgtgcagcctctggattcaccttcagtggctatg
ccatgagctgggtccgccaggctccagggaaggggctggagtgggtctcaaccattagtagtggcggaacctacatatac
taccccgactcagtgaagggccgattcaccatctccagagacaacgccaagaactcactgtatctgcaaatgaacagcct
gagagccgaggacacggccgtgtattactgtgcgagacttgggggggataattactacgaatacttcgatgtctggggca
aagggaccacggtcaccgtctcctccggcggtggcggatccggcggtggcggatccggcggtggcggatccgacattgtg
ctgacccagtctccagcctccttggccgtgtctccaggacagagggccaccatcacctgcagagccagtaagagtgtcag
taccagcggatactcctacatgcactggtatcagcagaaaccaggacaacctcctaaactcctgatttacctggcatcca
atctggagagcggggtcccagccaggttcagcggcagtgggtctgggaccgatttcaccctcacaattaatcctgtggaa
gctaatgatactgcaaattattactgtcagcacagtagggagctgcctttcacattcggcggagggaccaaggtggagat
caaacgaactgcacctgaactcctggggggaccgtcagtcttcctcttccccccaaaacccaaggacaccctcatgatct
cccggacccctgaggtcacatgcgtggtggtggacgtgagccacgaagaccctgaggtcaagttcaactggtacgtggac
ggcgtggaggtgcataatgccaagacaaagccgcgggaggagcagtacaacagcacgtaccgtgtggtcagcgtcctcac
cgtcctgcaccaggactggctgaatggcaaggagtacaagtgcaaggtctccaacaaagccctcccagcccccatcgaga
aaaccatctccaaagccaaagggcagccccgagaaccacaggtgtacaccctgcccccatcccgggaggagatgaccaag
aaccaggtcagcctgacctgcctggtcaaaggcttctatcccagcgacatcgccgtggagtgggagagcaatgggcagcc
ggagaacaactacaagaccacgcctcccgtgctggactccgacggctccttcttcctcaggagcaagctcaccgtggaca
agagcaggtggcagcaggggaacgtcttctcatgctccgtgatgcatgaggctctgcacaaccactacacgcagaagagc
ctctccctgtctccgggtaaaatctacatctgggcgcccttggccgggacttgtggggtccttctcctgtcactggttat
caccctttactgcaaacggggcagaaagaaactcctgtatatattcaaacaaccatttatgagaccagtacaaactactc
aagaggaagatggctgtagctgccgatttccagaagaagaagaaggaggatgtgaactgagagtgaagttcagcaggagc
gcagacgcccccgcgtacaagcagggccagaaccagctctataacgagctcaatctaggacgaagagaggagtacgatgt
tttggacaagagacgtggccgggaccctgagatggggggaaagccgagaaggaagaaccctcaggaaggcctgtacaatg
aactgcagaaagataagatggcggaggcctacagtgagattgggatgaaaggcgagcgccggaggggcaaggggcacgat
ggcctttaccagggtctcagtacagccaccaaggacacctacgacgcccttcacatgcaggccctgccccctcgctgata
a
(amino acids)
(SEQ ID NO: 737)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLRSKLTVDKSRWQQGNVFSCSVMHEALHNHYTQKS
LSLSPGKIYIWAPLAGTCGVLLLSLVITLYCKRGRKKLLYIFKQPFMRPVQTTQEEDGCSCRFPEEEEGGCELRVKFSRS
ADAPAYKQGQNQLYNELNLGRREEYDVLDKRRGRDPEMGGKPRRKNPQEGLYNELQKDKMAEAYSEIGMKGERRRGKGHD
GLYQGLSTATKDTYDALHMQALPPR**
CAR-T C2 IgD/FCHingeless Y407R/CD8/4-1BB/CD3z “IgD FcH” CAR56
N-CD8ls- huMNC2scFv- IgD hinge- Human IgG1 hingeless Fc Y407R- CD8
transmembrane- 4-1BB- CD3zeta-C
(DNA)
(SEQ ID NO: 738)
atggccttaccagtgaccgccttgctcctgccgctggccttgctgctccacgccgccaggccggaggtgcagctggtgga
gtctgggggaggcctggtcaagcctggggggtccctgagactctcctgtgcagcctctggattcaccttcagtggctatg
ccatgagctgggtccgccaggctccagggaaggggctggagtgggtctcaaccattagtagtggcggaacctacatatac
taccccgactcagtgaagggccgattcaccatctccagagacaacgccaagaactcactgtatctgcaaatgaacagcct
gagagccgaggacacggccgtgtattactgtgcgagacttgggggggataattactacgaatacttcgatgtctggggca
aagggaccacggtcaccgtctcctccggcggtggcggatccggcggtggcggatccggcggtggcggatccgacattgtg
ctgacccagtctccagcctccttggccgtgtctccaggacagagggccaccatcacctgcagagccagtaagagtgtcag
taccagcggatactcctacatgcactggtatcagcagaaaccaggacaacctcctaaactcctgatttacctggcatcca
atctggagagcggggtcccagccaggttcagcggcagtgggtctgggaccgatttcaccctcacaattaatcctgtggaa
gctaatgatactgcaaattattactgtcagcacagtagggagctgcctttcacattcggcggagggaccaaggtggagat
caaacgaactgagtctccaaaggcacaggcctcctcagtgcccactgcacaaccccaagcagagggcagcctcgccaagg
caaccacagccccagccaccacccgtaacacaggaagaggcggcgaagagaagaaaaaggagaaggagaaagaggaacaa
gaagagagagagacaaagacaccagcacctgaactcctggggggaccgtcagtcttcctcttccccccaaaacccaagga
caccctcatgatctcccggacccctgaggtcacatgcgtggtggtggacgtgagccacgaagaccctgaggtcaagttca
actggtacgtggacggcgtggaggtgcataatgccaagacaaagccgcgggaggagcagtacaacagcacgtaccgtgtg
gtcagcgtcctcaccgtcctgcaccaggactggctgaatggcaaggagtacaagtgcaaggtctccaacaaagccctccc
agcccccatcgagaaaaccatctccaaagccaaagggcagccccgagaaccacaggtgtacaccctgcccccatcccggg
aggagatgaccaagaaccaggtcagcctgacctgcctggtcaaaggcttctatcccagcgacatcgccgtggagtgggag
agcaatgggcagccggagaacaactacaagaccacgcctcccgtgctggactccgacggctccttcttcctcaggagcaa
gctcaccgtggacaagagcaggtggcagcaggggaacgtcttctcatgctccgtgatgcatgaggctctgcacaaccact
acacgcagaagagcctctccctgtctccgggtaaaatctacatctgggcgcccttggccgggacttgtggggtccttctc
ctgtcactggttatcaccctttactgcaaacggggcagaaagaaactcctgtatatattcaaacaaccatttatgagacc
agtacaaactactcaagaggaagatggctgtagctgccgatttccagaagaagaagaaggaggatgtgaactgagagtga
agttcagcaggagcgcagacgcccccgcgtacaagcagggccagaaccagctctataacgagctcaatctaggacgaaga
gaggagtacgatgttttggacaagagacgtggccgggaccctgagatggggggaaagccgagaaggaagaaccctcagga
aggcctgtacaatgaactgcagaaagataagatggcggaggcctacagtgagattgggatgaaaggcgagcgccggaggg
gcaaggggcacgatggcctttaccagggtctcagtacagccaccaaggacacctacgacgcccttcacatgcaggccctg
ccccctcgctgataa
(amino acids)
(SEQ ID NO: 739)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTESPKAQASSVPTAQPQAEGSLAKATTAPATTRNTGRGGEEKKKEKEKEEQ
EERETKTPAPELLGGPSVFLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSREEMTKNQVSLTCLVKGFYPSDIAVEWE
SNGQPENNYKTTPPVLDSDGSFFLRSKLTVDKSRWQQGNVFSCSVMHEALHNHYTQKSLSLSPGKIYIWAPLAGTCGVLL
LSLVITLYCKRGRKKLLYIFKQPFMRPVQTTQEEDGCSCRFPEEEEGGCELRVKFSRSADAPAYKQGQNQLYNELNLGRR
EEYDVLDKRRGRDPEMGGKPRRKNPQEGLYNELQKDKMAEAYSEIGMKGERRRGKGHDGLYQGLSTATKDTYDALHMQAL
PPR**
CAR-T C2 IgD/CD8/4-1BB/CD3z “IgD” CAR57
N-CD8ls-huMNC2scFv- IgD hinge- CD8 transmembrane- 4-1BB- CD3zeta-C
(DNA)
(SEQ ID NO: 740)
atggccttaccagtgaccgccttgctcctgccgctggccttgctgctccacgccgccaggccggaggtgcagctggtgga
gtctgggggaggcctggtcaagcctggggggtccctgagactctcctgtgcagcctctggattcaccttcagtggctatg
ccatgagctgggtccgccaggctccagggaaggggctggagtgggtctcaaccattagtagtggcggaacctacatatac
taccccgactcagtgaagggccgattcaccatctccagagacaacgccaagaactcactgtatctgcaaatgaacagcct
gagagccgaggacacggccgtgtattactgtgcgagacttgggggggataattactacgaatacttcgatgtctggggca
aagggaccacggtcaccgtctcctccggcggtggcggatccggcggtggcggatccggcggtggcggatccgacattgtg
ctgacccagtctccagcctccttggccgtgtctccaggacagagggccaccatcacctgcagagccagtaagagtgtcag
taccagcggatactcctacatgcactggtatcagcagaaaccaggacaacctcctaaactcctgatttacctggcatcca
atctggagagcggggtcccagccaggttcagcggcagtgggtctgggaccgatttcaccctcacaattaatcctgtggaa
gctaatgatactgcaaattattactgtcagcacagtagggagctgcctttcacattcggcggagggaccaaggtggagat
caaacgaactgagtctccaaaggcacaggcctcctcagtgcccactgcacaaccccaagcagagggcagcctcgccaagg
caaccacagccccagccaccacccgtaacacaggaagaggcggcgaagagaagaaaaaggagaaggagaaagaggaacaa
gaagagagagagacaaagacaccaatctacatctgggcgcccttggccgggacttgtggggtccttctcctgtcactggt
tatcaccctttactgcaaacggggcagaaagaaactcctgtatatattcaaacaaccatttatgagaccagtacaaacta
ctcaagaggaagatggctgtagctgccgatttccagaagaagaagaaggaggatgtgaactgagagtgaagttcagcagg
agcgcagacgcccccgcgtacaagcagggccagaaccagctctataacgagctcaatctaggacgaagagaggagtacga
tgttttggacaagagacgtggccgggaccctgagatggggggaaagccgagaaggaagaaccctcaggaaggcctgtaca
atgaactgcagaaagataagatggcggaggcctacagtgagattgggatgaaaggcgagcgccggaggggcaaggggcac
gatggcctttaccagggtctcagtacagccaccaaggacacctacgacgcccttcacatgcaggccctgccccctcgctg
ataa
(amino acids)
(SEQ ID NO: 741)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTESPKAQASSVPTAQPQAEGSLAKATTAPATTRNTGRGGEEKKKEKEKEEQ
EERETKTPIYIWAPLAGTCGVLLLSLVITLYCKRGRKKLLYIFKQPFMRPVQTTQEEDGCSCRFPEEEEGGCELRVKFSR
SADAPAYKQGQNQLYNELNLGRREEYDVLDKRRGRDPEMGGKPRRKNPQEGLYNELQKDKMAEAYSEIGMKGERRRGKGH
DGLYQGLSTATKDTYDALHMQALPPR**
CAR-T C2 X4/CD8/4-1BB/CD3z “X4” CAR58
N-CD8ls-huMNC2scFv- X4 linker- CD8 transmembrane- 4-1BB- CD3zeta-C
(DNA)
(SEQ ID NO: 742)
atggccttaccagtgaccgccttgctcctgccgctggccttgctgctccacgccgccaggccggaggtgcagctggtgga
gtctgggggaggcctggtcaagcctggggggtccctgagactctcctgtgcagcctctggattcaccttcagtggctatg
ccatgagctgggtccgccaggctccagggaaggggctggagtgggtctcaaccattagtagtggcggaacctacatatac
taccccgactcagtgaagggccgattcaccatctccagagacaacgccaagaactcactgtatctgcaaatgaacagcct
gagagccgaggacacggccgtgtattactgtgcgagacttgggggggataattactacgaatacttcgatgtctggggca
aagggaccacggtcaccgtctcctccggcggtggcggatccggcggtggcggatccggcggtggcggatccgacattgtg
ctgacccagtctccagcctccttggccgtgtctccaggacagagggccaccatcacctgcagagccagtaagagtgtcag
taccagcggatactcctacatgcactggtatcagcagaaaccaggacaacctcctaaactcctgatttacctggcatcca
atctggagagcggggtcccagccaggttcagcggcagtgggtctgggaccgatttcaccctcacaattaatcctgtggaa
gctaatgatactgcaaattattactgtcagcacagtagggagctgcctttcacattcggcggagggaccaaggtggagat
caaacgaactgacaagacgcacaccaagccacctaaaccagctccagaactgctcggaggtcctggcaccggaaccggag
gacctaccatcaaaccacctaagccacctaagcctgctcctaacctgctcggaggacctatctacatctgggcgcccttg
gccgggacttgtggggtccttctcctgtcactggttatcaccctttactgcaaacggggcagaaagaaactcctgtatat
attcaaacaaccatttatgagaccagtacaaactactcaagaggaagatggctgtagctgccgatttccagaagaagaag
aaggaggatgtgaactgagagtgaagttcagcaggagcgcagacgcccccgcgtacaagcagggccagaaccagctctat
aacgagctcaatctaggacgaagagaggagtacgatgttttggacaagagacgtggccgggaccctgagatggggggaaa
gccgagaaggaagaaccctcaggaaggcctgtacaatgaactgcagaaagataagatggcggaggcctacagtgagattg
ggatgaaaggcgagcgccggaggggcaaggggcacgatggcctttaccagggtctcagtacagccaccaaggacacctac
gacgcccttcacatgcaggccctgccccctcgctgataa
(amino acids)
(SEQ ID NO: 743)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTDKTHTKPPKPAPELLGGPGTGTGGPTIKPPKPPKPAPNLLGGPIYIWAPL
AGTCGVLLLSLVITLYCKRGRKKLLYIFKQPFMRPVQTTQEEDGCSCRFPEEEEGGCELRVKFSRSADAPAYKQGQNQLY
NELNLGRREEYDVLDKRRGRDPEMGGKPRRKNPQEGLYNELQKDKMAEAYSEIGMKGERRRGKGHDGLYQGLSTATKDTY
DALHMQALPPR**
CAR-T E6 CD8/CD4/41BB/CD3z CAR37
N-CD8ls-huMNE6scFv- CD8ecd- CD4 transmembrane- 4-1BB- CD3zeta-C
(DNA)
(SEQ ID NO: 744)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggcggaggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaaacgacaaccccggccccc
agaccaccaacgccagcccccaccatcgccagccaacccctgtctctgagaccagaagcctgtaggcctgccgccggtgga
gctgtgcacacaagaggactggatttcgcctgtgatatggccctgattgtgctggggggcgtcgccggcctcctgcttttc
attgggctaggcatcttcttcaaaaggggccgcaaaaaactcctttacatttttaagcagccttttatgaggccagtacag
acgactcaagaggaagacgggtgctcatgccgctttcctgaggaggaggaaggagggtgcgaactgcgcgttaagttctcc
cgatcagccgacgcgcctgcttacaagcagggccagaaccaactgtacaacgagctgaatctcggtagacgggaagagtac
gacgtgttggacaaacggagaggccgcgacccagaaatgggcggcaagcctcgcaggaaaaacccccaggagggactgtac
aatgagttgcagaaagataagatggcagaagcttatagcgagatcggaatgaagggggaaaggagacgagggaaaggacac
gacggcctttatcagggcctgtccacagcaacaaaagatacgtatgacgccctccatatgcaggcacttccaccacggtga
taa
(amino acids)
(SEQ ID NO: 745)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKTTTPAPRPPTPAPTIASQPLSLRPEACRPAAGGAVHTRGLDFACDMALIVLGGVAG
LLLFIGLGIFFKRGRKKLLYIFKQPFMRPVQTTQEEDGCSCRFPEEEEGGCELRVKFSRSADAPAYKQGQNQLYNELNLG
RREEYDVLDKRRGRDPEMGGKPRRKNPQEGLYNELQKDKMAEAYSEIGMKGERRRGKGHDGLYQGLSTATKDTYDALHMQ
ALPPR**
CAR-T E6 CD4/CD4/CD3z sequence CAR23:
N-CD8ls-huMNE6scFv- CD4ecd- CD4 transmembrane- CD3zeta-C
(DNA)
(SEQ ID NO: 746)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggggggggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaatcgggacaggtcctgctg
gaatccaacatcaaggttctgcccacatggtccaccccggtgcagccaatggccctgattgtgctggggggcgtcgccggc
ctcctgcttttcattgggctaggcatcttcttccgcgttaagttctcccgatcagccgacgcgcctgcttacaagcagggc
cagaaccaactgtacaacgagctgaatctcggtagacgggaagagtacgacgtgttggacaaacggagaggccgcgaccca
gaaatgggcggcaagcctcgcaggaaaaacccccaggagggactgtacaatgagttgcagaaagataagatggcagaagct
tatagcgagatcggaatgaagggggaaaggagacgagggaaaggacacgacggcctttatcagggcctgtccacagcaaca
aaagatacgtatgacgccctccatatgcaggcacttccaccacggtgataa
(amino acids)
(SEQ ID NO: 747)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKSGQVLLESNIKVLPTWSTPVQPMALIVLGGVAGLLLFIGLGIFFRVKFSRSADAPA
YKQGQNQLYNELNLGRREEYDVLDKRRGRDPEMGGKPRRKNPQEGLYNELQKDKMAEAYSEIGMKGERRRGKGHDGLYQG
LSTATKDTYDALHMQALPPR**
CAR-T E6 CD4/CD4/CD28/CD3z sequence CAR25:
N-CD8ls-huMNE6scFv- CD4ecd- CD4 transmembrane- CD28- CD3zeta-C
(DNA)
(SEQ ID NO: 748)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggggggggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaatcgggacaggtcctgctg
gaatccaacatcaaggttctgcccacatggtccaccccggtgcagccaatggccctgattgtgctggggggcgtcgccggc
ctcctgcttttcattgggctaggcatcttcttcagaagcaagcggtctcggctcctgcattctgattacatgaacatgacc
ccaagaagaccaggccccaccaggaaacattaccagccctacgctccgccacgcgacttcgctgcctaccggtcccgcgtt
aagttctcccgatcagccgacgcgcctgcttacaagcagggccagaaccaactgtacaacgagctgaatctcggtagacgg
gaagagtacgacgtgttggacaaacggagaggccgcgacccagaaatgggcggcaagcctcgcaggaaaaacccccaggag
ggactgtacaatgagttgcagaaagataagatggcagaagcttatagcgagatcggaatgaagggggaaaggagacgaggg
aaaggacacgacggcctttatcagggcctgtccacagcaacaaaagatacgtatgacgccctccatatgcaggcacttcca
ccacggtgataa
(amino acids)
(SEQ ID NO: 749)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKSGQVLLESNIKVLPTWSTPVQPMALIVLGGVAGLLLFIGLGIFFRSKRSRLLHSDY
MNMTPRRPGPTRKHYQPYAPPRDFAAYRSRVKFSRSADAPAYKQGQNQLYNELNLGRREEYDVLDKRRGRDPEMGGKPRR
KNPQEGLYNELQKDKMAEAYSEIGMKGERRRGKGHDGLYQGLSTATKDTYDALHMQALPPR**
CAR-T E6 CD4/CD4/4-1BB/CD3z sequence CAR31:
N-CD8ls-huMNE6scFv- CD4ecd- CD4 transmembrane- 4-1BB- CD3zeta-C
(DNA)
(SEQ ID NO: 750)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggggggggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaatcgggacaggtcctgctg
gaatccaacatcaaggttctgcccacatggtccaccccggtgcagccaatggccctgattgtgctggggggcgtcgccggc
ctcctgcttttcattgggctaggcatcttcttcaaaaggggccgcaaaaaactcctttacatttttaagcagccttttatg
aggccagtacagacgactcaagaggaagacgggtgctcatgccgctttcctgaggaggaggaaggagggtgcgaactgcgc
gttaagttctcccgatcagccgacgcgcctgcttacaagcagggccagaaccaactgtacaacgagctgaatctcggtaga
cgggaagagtacgacgtgttggacaaacggagaggccgcgacccagaaatgggcggcaagcctcgcaggaaaaacccccag
gagggactgtacaatgagttgcagaaagataagatggcagaagcttatagcgagatcggaatgaagggggaaaggagacga
gggaaaggacacgacggcctttatcagggcctgtccacagcaacaaaagatacgtatgacgccctccatatgcaggcactt
ccaccacggtgataa
(amino acids)
(SEQ ID NO: 751)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKSGQVLLESNIKVLPTWSTPVQPMALIVLGGVAGLLLFIGLGIFFKRGRKKLLYIFK
QPFMRPVQTTQEEDGCSCRFPEEEEGGCELRVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDTYDALHMQALPPR**
CAR-T E6 CD4/CD4/OX40/CD3z sequence:
N-CD8ls-huMNE6scFv- CD4ecd- CD4 transmembrane- OX40- CD3zeta-C
(DNA)
(SEQ ID NO: 752)
atggccctgcccgtgaccgctttgctgctccccctggcgctgctgctgcacgccgccaggccagaggtccagctggttga
gagtggcggtgggctggttaagcctggcggctccctgcggctgagctgcgccgcgagtggatttactttcagccgatatg
ggatgagttgggtgcggcaagctcccgggaagaggctggaatgggtctcaacaatctccggggggggcacttacatctat
taccccgactcagtcaaggggagatttaccatttcacgagacaacgctaagaataccctgtatttgcagatgaattctct
gagagcagaggacacagctgtttactattgtacccgcgacaactatggcaggaactacgactacggtatggactattggg
gacaagggacattggttacagtgagcagtggcggcgggggcagcggaggaggaggcagcggtggggggggcagcgagata
gtgctcacgcagtcacccgcgactctcagtctctcacctggggaacgagctaccctgacgtgctctgctacctcctcagt
gtcatatattcactggtatcagcaacggcccgggcagtcccctagattgctcatttatagtacctctaatctggcctcag
gtatccctgcacgattttctggatctggttcaggttctgattacaccctcactatctctagcctggagcctgaagacttt
gccgtttattactgccagcagaggtctagctccccattcacctttgggagtgggaccaaggttgaaattaaatcgggaca
ggtcctgctggaatccaacatcaaggttctgcccacatggtccaccccggtgcagccaatggccctgattgtgctggggg
gcgtcgccggcctcctgcttttcattgggctaggcatcttcttccggagggaccagaggctgccccccgatgcccacaag
ccccctgggggaggcagtttccggacccccatccaagaggagcaggccgacgcccactccaccctggccaagatccgcgt
taagttctcccgatcagccgacgcgcctgcttacaagcagggccagaaccaactgtacaacgagctgaatctcggtagac
gggaagagtacgacgtgttggacaaacggagaggccgcgacccagaaatgggcggcaagcctcgcaggaaaaacccccag
gagggactgtacaatgagttgcagaaagataagatggcagaagcttatagcgagatcggaatgaagggggaaaggagacg
agggaaaggacacgacggcctttatcagggcctgtccacagcaacaaaagatacgtatgacgccctccatatgcaggcac
ttccaccacggtgataa
(amino acids)
(SEQ ID NO: 753)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKSGQVLLESNIKVLPTWSTPVQPMALIVLGGVAGLLLFIGLGIFFRRDQRLPPDAHK
PPGGGSFRTPIQEEQADAHSTLAKIRVKFSRSADAPAYKQGQNQLYNELNLGRREEYDVLDKRRGRDPEMGGKPRRKNPQ
EGLYNELQKDKMAEAYSEIGMKGERRRGKGHDGLYQGLSTATKDTYDALHMQALPPR**
CAR-T E6 CD4/CD4/CD28/4-1BB/CD3z sequence CAR38:
N-CD8ls-huMNE6scFv- CD4ecd- CD4 transmembrane- CD28- 4-1BB- CD3zeta-C
(DNA)
(SEQ ID NO: 754)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggggggggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaatcgggacaggtcctgctg
gaatccaacatcaaggttctgcccacatggtccaccccggtgcagccaatggccctgattgtgctggggggcgtcgccggc
ctcctgcttttcattgggctaggcatcttcttcagaagcaagcggtctcggctcctgcattctgattacatgaacatgacc
ccaagaagaccaggccccaccaggaaacattaccagccctacgctccgccacgcgacttcgctgcctaccggtccaaaagg
ggccgcaaaaaactcctttacatttttaagcagccttttatgaggccagtacagacgactcaagaggaagacgggtgctca
tgccgctttcctgaggaggaggaaggagggtgcgaactgcgcgttaagttctcccgatcagccgacgcgcctgcttacaag
cagggccagaaccaactgtacaacgagctgaatctcggtagacgggaagagtacgacgtgttggacaaacggagaggccgc
gacccagaaatgggcggcaagcctcgcaggaaaaacccccaggagggactgtacaatgagttgcagaaagataagatggca
gaagcttatagcgagatcggaatgaagggggaaaggagacgagggaaaggacacgacggcctttatcagggcctgtccaca
gcaacaaaagatacgtatgacgccctccatatgcaggcacttccaccacggtgataa
(amino acids)
(SEQ ID NO: 755)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKSGQVLLESNIKVLPTWSTPVQPMALIVLGGVAGLLLFIGLGIFFRSKRSRLLHSDY
MNMTPRRPGPTRKHYQPYAPPRDFAAYRSKRGRKKLLYIFKQPFMRPVQTTQEEDGCSCRFPEEEEGGCELRVKFSRSAD
APAYKQGQNQLYNELNLGRREEYDVLDKRRGRDPEMGGKPRRKNPQEGLYNELQKDKMAEAYSEIGMKGERRRGKGHDGL
YQGLSTATKDTYDALHMQALPPR**
CAR-T E6 CD4/CD4/CD28/OX40/CD3z sequence:
N-CD8ls-huMNE6scFv- CD4ecd- CD4 transmembrane- CD28- OX40- CD3zeta-C
(DNA)
(SEQ ID NO: 756)
atggccctgcccgtgaccgctttgctgctccccctggcgctgctgctgcacgccgccaggccagaggtccagctggttga
gagtggcggtgggctggttaagcctggcggctccctgcggctgagctgcgccgcgagtggatttactttcagccgatatg
ggatgagttgggtgcggcaagctcccgggaagaggctggaatgggtctcaacaatctccggggggggcacttacatctat
taccccgactcagtcaaggggagatttaccatttcacgagacaacgctaagaataccctgtatttgcagatgaattctct
gagagcagaggacacagctgtttactattgtacccgcgacaactatggcaggaactacgactacggtatggactattggg
gacaagggacattggttacagtgagcagtggcggcgggggcagcggaggaggaggcagcggtggggggggcagcgagata
gtgctcacgcagtcacccgcgactctcagtctctcacctggggaacgagctaccctgacgtgctctgctacctcctcagt
gtcatatattcactggtatcagcaacggcccgggcagtcccctagattgctcatttatagtacctctaatctggcctcag
gtatccctgcacgattttctggatctggttcaggttctgattacaccctcactatctctagcctggagcctgaagacttt
gccgtttattactgccagcagaggtctagctccccattcacctttgggagtgggaccaaggttgaaattaaatcgggaca
ggtcctgctggaatccaacatcaaggttctgcccacatggtccaccccggtgcagccaatggccctgattgtgctggggg
gcgtcgccggcctcctgcttttcattgggctaggcatcttcttcagaagcaagcggtctcggctcctgcattctgattac
atgaacatgaccccaagaagaccaggccccaccaggaaacattaccagccctacgctccgccacgcgacttcgctgccta
ccggtcccggagggaccagaggctgccccccgatgcccacaagccccctgggggaggcagtttccggacccccatccaag
aggagcaggccgacgcccactccaccctggccaagatccgcgttaagttctcccgatcagccgacgcgcctgcttacaag
cagggccagaaccaactgtacaacgagctgaatctcggtagacgggaagagtacgacgtgttggacaaacggagaggccg
cgacccagaaatgggcggcaagcctcgcaggaaaaacccccaggagggactgtacaatgagttgcagaaagataagatgg
cagaagcttatagcgagatcggaatgaagggggaaaggagacgagggaaaggacacgacggcctttatcagggcctgtcc
acagcaacaaaagatacgtatgacgccctccatatgcaggcacttccaccacggtgataa
(amino acids)
(SEQ ID NO: 757)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKSGQVLLESNIKVLPTWSTPVQPMALIVLGGVAGLLLFIGLGIFFRSKRSRLLHSDY
MNMTPRRPGPTRKHYQPYAPPRDFAAYRSRRDQRLPPDAHKPPGGGSFRTPIQEEQADAHSTLAKIRVKFSRSADAPAYK
QGQNQLYNELNLGRREEYDVLDKRRGRDPEMGGKPRRKNPQEGLYNELQKDKMAEAYSEIGMKGERRRGKGHDGLYQGLS
TATKDTYDALHMQALPPR**
CAR-T C2 CD4/CD4/CD3z sequence:
N-CD8ls-huMNC2scFv- CD4ecd- CD4 transmembrane- CD3zeta-C
(DNA)
(SEQ ID NO: 758)
atggccttgccagtgacggccctgctgctgccattggctcttctgttgcacgctgccaggcctgaagtgcagctcgtagag
agtggcgggggactggtgaagcccggtggaagcctcagactcagttgcgccgcctcaggtttcactttttcaggttacgcc
atgtcctgggtaagacaggcaccggggaaaggactcgagtgggtgtctactatcagctcaggaggcacttatatatattat
cctgactctgtaaaaggccgatttacgatttctcgcgacaatgcaaagaactccctctacctccaaatgaacagtcttagg
gcagaagacactgctgtatactattgtgcacgcctcggcggcgacaactactacgagtactttgacgtgtgggggaaaggg
actaccgtgacagtttcaagcggaggaggtggctcaggtggaggcgggtcaggggggggaggaagtgatattgtgctcaca
caatccccagcctccctggctgtgtctcccggccaacgcgctacaattacatgtcgggcctccaaaagcgtgagcaccagc
ggctacagctacatgcactggtatcaacagaaaccaggacaaccccccaaactgttgatttatctcgcttcaaacttggag
tccggcgtgcctgcgcgcttttcagggagtgggagcggcacagattttacgctgactatcaaccccgtagaagcaaacgat
acagcgaattattattgtcaacattcccgggaactcccctttacgttcggcgggggcacaaaggtcgaaattaagagaacc
tcgggacaggtcctgctggaatccaacatcaaggttctgcccacatggtccaccccggtgcagccaatggccctgattgtg
ctggggggcgtcgccggcctcctgcttttcattgggctaggcatcttcttccgcgttaagttctcccgatcagccgacgcg
cctgcttacaagcagggccagaaccaactgtacaacgagctgaatctcggtagacgggaagagtacgacgtgttggacaaa
cggagaggccgcgacccagaaatgggcggcaagcctcgcaggaaaaacccccaggagggactgtacaatgagttgcagaaa
gataagatggcagaagcttatagcgagatcggaatgaagggggaaaggagacgagggaaaggacacgacggcctttatcag
ggcctgtccacagcaacaaaagatacgtatgacgccctccatatgcaggcacttccaccacggtgataa
(amino acids)
(SEQ ID NO: 759)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTSGQVLLESNIKVLPTWSTPVQPMALIVLGGVAGLLLFIGLGIFFRVKFSR
SADAPAYKQGQNQLYNELNLGRREEYDVLDKRRGRDPEMGGKPRRKNPQEGLYNELQKDKMAEAYSEIGMKGERRRGKGH
DGLYQGLSTATKDTYDALHMQALPPR**
CAR-T C2 CD4/CD4/CD28/CD3z sequence:
N-CD8ls-huMNC2scFv- CD4ecd- CD4 transmembrane- CD28- CD3zeta-C
(DNA)
(SEQ ID NO: 760)
atggccttgccagtgacggccctgctgctgccattggctcttctgttgcacgctgccaggcctgaagtgcagctcgtaga
gagtggcgggggactggtgaagcccggtggaagcctcagactcagttgcgccgcctcaggtttcactttttcaggttacg
ccatgtcctgggtaagacaggcaccggggaaaggactcgagtgggtgtctactatcagctcaggaggcacttatatatat
tatcctgactctgtaaaaggccgatttacgatttctcgcgacaatgcaaagaactccctctacctccaaatgaacagtct
tagggcagaagacactgctgtatactattgtgcacgcctcggcggcgacaactactacgagtactttgacgtgtggggga
aagggactaccgtgacagtttcaagcggaggaggtggctcaggtggaggcgggtcaggggggggaggaagtgatattgtg
ctcacacaatccccagcctccctggctgtgtctcccggccaacgcgctacaattacatgtcgggcctccaaaagcgtgag
caccagcggctacagctacatgcactggtatcaacagaaaccaggacaaccccccaaactgttgatttatctcgcttcaa
acttggagtccggcgtgcctgcgcgcttttcagggagtgggagcggcacagattttacgctgactatcaaccccgtagaa
gcaaacgatacagcgaattattattgtcaacattcccgggaactcccctttacgttcggcgggggcacaaaggtcgaaat
taagagaacctcgggacaggtcctgctggaatccaacatcaaggttctgcccacatggtccaccccggtgcagccaatgg
ccctgattgtgctggggggcgtcgccggcctcctgcttttcattgggctaggcatcttcttcagaagcaagcggtctcgg
ctcctgcattctgattacatgaacatgaccccaagaagaccaggccccaccaggaaacattaccagccctacgctccgcc
acgcgacttcgctgcctaccggtcccgcgttaagttctcccgatcagccgacgcgcctgcttacaagcagggccagaacc
aactgtacaacgagctgaatctcggtagacgggaagagtacgacgtgttggacaaacggagaggccgcgacccagaaatg
ggcggcaagcctcgcaggaaaaacccccaggagggactgtacaatgagttgcagaaagataagatggcagaagcttatag
cgagatcggaatgaagggggaaaggagacgagggaaaggacacgacggcctttatcagggcctgtccacagcaacaaaag
atacgtatgacgccctccatatgcaggcacttccaccacggtgataa
(amino acids)
(SEQ ID NO: 761)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTSGQVLLESNIKVLPTWSTPVQPMALIVLGGVAGLLLFIGLGIFFRSKRSR
LLHSDYMNMTPRRPGPTRKHYQPYAPPRDFAAYRSRVKFSRSADAPAYKQGQNQLYNELNLGRREEYDVLDKRRGRDPEM
GGKPRRKNPQEGLYNELQKDKMAEAYSEIGMKGERRRGKGHDGLYQGLSTATKDTYDALHMQALPPR**
CAR-T C2 CD4/CD4/4-1BB/CD3z sequence:
N-CD8ls-huMNC2scFv- CD4ecd- CD4 transmembrane- 4-1BB- CD3zeta-C
(DNA)
(SEQ ID NO: 762)
atggccttgccagtgacggccctgctgctgccattggctcttctgttgcacgctgccaggcctgaagtgcagctcgtagag
agtggcgggggactggtgaagcccggtggaagcctcagactcagttgcgccgcctcaggtttcactttttcaggttacgcc
atgtcctgggtaagacaggcaccggggaaaggactcgagtgggtgtctactatcagctcaggaggcacttatatatattat
cctgactctgtaaaaggccgatttacgatttctcgcgacaatgcaaagaactccctctacctccaaatgaacagtcttagg
gcagaagacactgctgtatactattgtgcacgcctcggcggcgacaactactacgagtactttgacgtgtgggggaaaggg
actaccgtgacagtttcaagcggaggaggtggctcaggtggaggcgggtcaggggggggaggaagtgatattgtgctcaca
caatccccagcctccctggctgtgtctcccggccaacgcgctacaattacatgtcgggcctccaaaagcgtgagcaccagc
ggctacagctacatgcactggtatcaacagaaaccaggacaaccccccaaactgttgatttatctcgcttcaaacttggag
tccggcgtgcctgcgcgcttttcagggagtgggagcggcacagattttacgctgactatcaaccccgtagaagcaaacgat
acagcgaattattattgtcaacattcccgggaactcccctttacgttcggcgggggcacaaaggtcgaaattaagagaacc
tcgggacaggtcctgctggaatccaacatcaaggttctgcccacatggtccaccccggtgcagccaatggccctgattgtg
ctggggggcgtcgccggcctcctgcttttcattgggctaggcatcttcttcaaaaggggccgcaaaaaactcctttacatt
tttaagcagccttttatgaggccagtacagacgactcaagaggaagacgggtgctcatgccgctttcctgaggaggaggaa
ggagggtgcgaactgcgcgttaagttctcccgatcagccgacgcgcctgcttacaagcagggccagaaccaactgtacaac
gagctgaatctcggtagacgggaagagtacgacgtgttggacaaacggagaggccgcgacccagaaatgggcggcaagcct
cgcaggaaaaacccccaggagggactgtacaatgagttgcagaaagataagatggcagaagcttatagcgagatcggaatg
aagggggaaaggagacgagggaaaggacacgacggcctttatcagggcctgtccacagcaacaaaagatacgtatgacgcc
ctccatatgcaggcacttccaccacggtgataa
(amino acids)
(SEQ ID NO: 763)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTSGQVLLESNIKVLPTWSTPVQPMALIVLGGVAGLLLFIGLGIFFKRGRKK
LLYIFKQPFMRPVQTTQEEDGCSCRFPEEEEGGCELRVKFSRSADAPAYKQGQNQLYNELNLGRREEYDVLDKRRGRDPE
MGGKPRRKNPQEGLYNELQKDKMAEAYSEIGMKGERRRGKGHDGLYQGLSTATKDTYDALHMQALPPR**
CAR-T C2 CD4/CD4/OX40/CD3z sequence:
N-CD8ls-huMNC2scFv- CD4ecd- CD4 transmembrane- OX40- CD3zeta-C
(DNA)
(SEQ ID NO: 764)
atggccttgccagtgacggccctgctgctgccattggctcttctgttgcacgctgccaggcctgaagtgcagctcgtaga
gagtggcgggggactggtgaagcccggtggaagcctcagactcagttgcgccgcctcaggtttcactttttcaggttacg
ccatgtcctgggtaagacaggcaccggggaaaggactcgagtgggtgtctactatcagctcaggaggcacttatatatat
tatcctgactctgtaaaaggccgatttacgatttctcgcgacaatgcaaagaactccctctacctccaaatgaacagtct
tagggcagaagacactgctgtatactattgtgcacgcctcggcggcgacaactactacgagtactttgacgtgtggggga
aagggactaccgtgacagtttcaagcggaggaggtggctcaggtggaggcgggtcaggggggggaggaagtgatattgtg
ctcacacaatccccagcctccctggctgtgtctcccggccaacgcgctacaattacatgtcgggcctccaaaagcgtgag
caccagcggctacagctacatgcactggtatcaacagaaaccaggacaaccccccaaactgttgatttatctcgcttcaa
acttggagtccggcgtgcctgcgcgcttttcagggagtgggagcggcacagattttacgctgactatcaaccccgtagaa
gcaaacgatacagcgaattattattgtcaacattcccgggaactcccctttacgttcggcgggggcacaaaggtcgaaat
taagagaacctcgggacaggtcctgctggaatccaacatcaaggttctgcccacatggtccaccccggtgcagccaatgg
ccctgattgtgctggggggcgtcgccggcctcctgcttttcattgggctaggcatcttcttccggagggaccagaggctg
ccccccgatgcccacaagccccctgggggaggcagtttccggacccccatccaagaggagcaggccgacgcccactccac
cctggccaagatccgcgttaagttctcccgatcagccgacgcgcctgcttacaagcagggccagaaccaactgtacaacg
agctgaatctcggtagacgggaagagtacgacgtgttggacaaacggagaggccgcgacccagaaatgggcggcaagcct
cgcaggaaaaacccccaggagggactgtacaatgagttgcagaaagataagatggcagaagcttatagcgagatcggaat
gaagggggaaaggagacgagggaaaggacacgacggcctttatcagggcctgtccacagcaacaaaagatacgtatgacg
ccctccatatgcaggcacttccaccacggtgataa
(amino acids)
(SEQ ID NO: 765)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTSGQVLLESNIKVLPTWSTPVQPMALIVLGGVAGLLLFIGLGIFFRRDQRL
PPDAHKPPGGGSFRTPIQEEQADAHSTLAKIRVKFSRSADAPAYKQGQNQLYNELNLGRREEYDVLDKRRGRDPEMGGKP
RRKNPQEGLYNELQKDKMAEAYSEIGMKGERRRGKGHDGLYQGLSTATKDTYDALHMQALPPR**
CAR-T C2 CD4/CD4/CD28/4-1BB/CD3z sequence:
N-CD8ls-huMNC2scFv- CD4ecd- CD4 transmembrane- CD28- 4-1BB- CD3zeta-C
(DNA)
(SEQ ID NO: 766)
atggccttgccagtgacggccctgctgctgccattggctcttctgttgcacgctgccaggcctgaagtgcagctcgtaga
gagtggcgggggactggtgaagcccggtggaagcctcagactcagttgcgccgcctcaggtttcactttttcaggttacg
ccatgtcctgggtaagacaggcaccggggaaaggactcgagtgggtgtctactatcagctcaggaggcacttatatatat
tatcctgactctgtaaaaggccgatttacgatttctcgcgacaatgcaaagaactccctctacctccaaatgaacagtct
tagggcagaagacactgctgtatactattgtgcacgcctcggcggcgacaactactacgagtactttgacgtgtggggga
aagggactaccgtgacagtttcaagcggaggaggtggctcaggtggaggcgggtcaggggggggaggaagtgatattgtg
ctcacacaatccccagcctccctggctgtgtctcccggccaacgcgctacaattacatgtcgggcctccaaaagcgtgag
caccagcggctacagctacatgcactggtatcaacagaaaccaggacaaccccccaaactgttgatttatctcgcttcaa
acttggagtccggcgtgcctgcgcgcttttcagggagtgggagcggcacagattttacgctgactatcaaccccgtagaa
gcaaacgatacagcgaattattattgtcaacattcccgggaactcccctttacgttcggcgggggcacaaaggtcgaaat
taagagaacctcgggacaggtcctgctggaatccaacatcaaggttctgcccacatggtccaccccggtgcagccaatgg
ccctgattgtgctggggggcgtcgccggcctcctgcttttcattgggctaggcatcttcttcagaagcaagcggtctcgg
ctcctgcattctgattacatgaacatgaccccaagaagaccaggccccaccaggaaacattaccagccctacgctccgcc
acgcgacttcgctgcctaccggtccaaaaggggccgcaaaaaactcctttacatttttaagcagccttttatgaggccag
tacagacgactcaagaggaagacgggtgctcatgccgctttcctgaggaggaggaaggagggtgcgaactgcgcgttaag
ttctcccgatcagccgacgcgcctgcttacaagcagggccagaaccaactgtacaacgagctgaatctcggtagacggga
agagtacgacgtgttggacaaacggagaggccgcgacccagaaatgggcggcaagcctcgcaggaaaaacccccaggagg
gactgtacaatgagttgcagaaagataagatggcagaagcttatagcgagatcggaatgaagggggaaaggagacgaggg
aaaggacacgacggcctttatcagggcctgtccacagcaacaaaagatacgtatgacgccctccatatgcaggcacttcc
accacggtgataa
(amino acids)
(SEQ ID NO: 767)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTSGQVLLESNIKVLPTWSTPVQPMALIVLGGVAGLLLFIGLGIFFRSKRSR
LLHSDYMNMTPRRPGPTRKHYQPYAPPRDFAAYRSKRGRKKLLYIFKQPFMRPVQTTQEEDGCSCRFPEEEEGGCELRVK
FSRSADAPAYKQGQNQLYNELNLGRREEYDVLDKRRGRDPEMGGKPRRKNPQEGLYNELQKDKMAEAYSEIGMKGERRRG
KGHDGLYQGLSTATKDTYDALHMQALPPR**
CAR-T C2 CD4/CD4/CD28/OX40/CD3z sequence:
N-CD8ls-huMNC2scFv- CD4ecd- CD4 transmembrane- CD28- OX40- CD3zeta-C
(DNA)
(SEQ ID NO: 768)
atggccttgccagtgacggccctgctgctgccattggctcttctgttgcacgctgccaggcctgaagtgcagctcgtaga
gagtggcgggggactggtgaagcccggtggaagcctcagactcagttgcgccgcctcaggtttcactttttcaggttacg
ccatgtcctgggtaagacaggcaccggggaaaggactcgagtgggtgtctactatcagctcaggaggcacttatatatat
tatcctgactctgtaaaaggccgatttacgatttctcgcgacaatgcaaagaactccctctacctccaaatgaacagtct
tagggcagaagacactgctgtatactattgtgcacgcctcggcggcgacaactactacgagtactttgacgtgtggggga
aagggactaccgtgacagtttcaagcggaggaggtggctcaggtggaggcgggtcaggggggggaggaagtgatattgtg
ctcacacaatccccagcctccctggctgtgtctcccggccaacgcgctacaattacatgtcgggcctccaaaagcgtgag
caccagcggctacagctacatgcactggtatcaacagaaaccaggacaaccccccaaactgttgatttatctcgcttcaa
acttggagtccggcgtgcctgcgcgcttttcagggagtgggagcggcacagattttacgctgactatcaaccccgtagaa
gcaaacgatacagcgaattattattgtcaacattcccgggaactcccctttacgttcggcgggggcacaaaggtcgaaat
taagagaacctcgggacaggtcctgctggaatccaacatcaaggttctgcccacatggtccaccccggtgcagccaatgg
ccctgattgtgctggggggcgtcgccggcctcctgcttttcattgggctaggcatcttcttcagaagcaagcggtctcgg
ctcctgcattctgattacatgaacatgaccccaagaagaccaggccccaccaggaaacattaccagccctacgctccgcc
acgcgacttcgctgcctaccggtcccggagggaccagaggctgccccccgatgcccacaagccccctgggggaggcagtt
tccggacccccatccaagaggagcaggccgacgcccactccaccctggccaagatccgcgttaagttctcccgatcagcc
gacgcgcctgcttacaagcagggccagaaccaactgtacaacgagctgaatctcggtagacgggaagagtacgacgtgtt
ggacaaacggagaggccgcgacccagaaatgggcggcaagcctcgcaggaaaaacccccaggagggactgtacaatgagt
tgcagaaagataagatggcagaagcttatagcgagatcggaatgaagggggaaaggagacgagggaaaggacacgacggc
ctttatcagggcctgtccacagcaacaaaagatacgtatgacgccctccatatgcaggcacttccaccacggtgataa
(amino acids)
(SEQ ID NO: 769)
MALPVTALLLPLALLLHAARPEVQLVESGGGLVKPGGSLRLSCAASGFTFSGYAMSWVRQAPGKGLEWVSTISSGGTYIY
YPDSVKGRFTISRDNAKNSLYLQMNSLRAEDTAVYYCARLGGDNYYEYFDVWGKGTTVTVSSGGGGSGGGGSGGGGSDIV
LTQSPASLAVSPGQRATITCRASKSVSTSGYSYMHWYQQKPGQPPKLLIYLASNLESGVPARFSGSGSGTDFTLTINPVE
ANDTANYYCQHSRELPFTFGGGTKVEIKRTSGQVLLESNIKVLPTWSTPVQPMALIVLGGVAGLLLFIGLGIFFRSKRSR
LLHSDYMNMTPRRPGPTRKHYQPYAPPRDFAAYRSRRDQRLPPDAHKPPGGGSFRTPIQEEQADAHSTLAKIRVKFSRSA
DAPAYKQGQNQLYNELNLGRREEYDVLDKRRGRDPEMGGKPRRKNPQEGLYNELQKDKMAEAYSEIGMKGERRRGKGHDG
LYQGLSTATKDTYDALHMQALPPR**
CAR-T E6 IgD/FC/CD8/4-1BB/CD3z
N-CD8ls-huMNE6scFv- IgD hinge- Human IgG1 Fc- CD8 transmembrane- 4-1BB- CD3zeta-
C
(DNA)
(SEQ ID NO: 770)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggcggaggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaagagtctccaaaggcacag
gcctcctcagtgcccactgcacaaccccaagcagagggcagcctcgccaaggcaaccacagccccagccaccacccgtaac
acaggaagaggcggcgaagagaagaaaaaggagaaggagaaagaggaacaagaagagagagagacaaagacaccagagccc
aaatcttgtgacaaaactcacacatgcccaccgtgcccagcacctgaactcctggggggaccgtcagtcttcctcttcccc
ccaaaacccaaggacaccctcatgatctcccggacccctgaggtcacatgcgtggtggtggacgtgagccacgaagaccct
gaggtcaagttcaactggtacgtggacggcgtggaggtgcataatgccaagacaaagccgcgggaggagcagtacaacagc
acgtaccgtgtggtcagcgtcctcaccgtcctgcaccaggactggctgaatggcaaggagtacaagtgcaaggtctccaac
aaagccctcccagcccccatcgagaaaaccatctccaaagccaaagggcagccccgagaaccacaggtgtacaccctgccc
ccatcccgggaggagatgaccaagaaccaggtcagcctgacctgcctggtcaaaggcttctatcccagcgacatcgccgtg
gagtgggagagcaatgggcagccggagaacaactacaagaccacgcctcccgtgctggactccgacggctccttcttcctc
tacagcaagctcaccgtggacaagagcaggtggcagcaggggaacgtcttctcatgctccgtgatgcatgaggctctgcac
aaccactacacgcagaagagcctctccctgtctccgggtaaaatctacatctgggcgcccttggccgggacttgtggggtc
cttctcctgtcactggttatcaccctttactgcaaacggggcagaaagaaactcctgtatatattcaaacaaccatttatg
agaccagtacaaactactcaagaggaagatggctgtagctgccgatttccagaagaagaagaaggaggatgtgaactgaga
gtgaagttcagcaggagcgcagacgcccccgcgtacaagcagggccagaaccagctctataacgagctcaatctaggacga
agagaggagtacgatgttttggacaagagacgtggccgggaccctgagatggggggaaagccgagaaggaagaaccctcag
gaaggcctgtacaatgaactgcagaaagataagatggcggaggcctacagtgagattgggatgaaaggcgagcgccggagg
ggcaaggggcacgatggcctttaccagggtctcagtacagccaccaaggacacctacgacgcccttcacatgcaggccctg
ccccctcgctgataa
(amino acids)
(SEQ ID NO: 771)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKESPKAQASSVPTAQPQAEGSLAKATTAPATTRNTGRGGEEKKKEKEKEEQEERETK
TPEPKSCDKTHTCPPCPAPELLGGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNVFSCSVMHEALHNHYTQKSLSLSPGKIYIWAP
LAGTCGVLLLSLVITLYCKRGRKKLLYIFKQPFMRPVQTTQEEDGCSCRFPEEEEGGCELRVKFSRSADAPAYKQGQNQL
YNELNLGRREEYDVLDKRRGRDPEMGGKPRRKNPQEGLYNELQKDKMAEAYSEIGMKGERRRGKGHDGLYQGLSTATKDT
YDALHMQALPPR**
CAR-T E6 IgD/FCHingeless Y407R/CD8/4-1BB/CD3z
N-CD8ls- huMNE6scFv- IgD hinge- Human IgG1 hingeless Fc Y407R- CD8
transmembrane- 4-1BB- CD3zeta-C
(DNA)
(SEQ ID NO: 772)
atggccctgcccgtgaccgctttgctgctccccctggcgctgctgctgcacgccgccaggccagaggtccagctggttgag
agtggcggtgggctggttaagcctggcggctccctgcggctgagctgcgccgcgagtggatttactttcagccgatatggg
atgagttgggtgcggcaagctcccgggaagaggctggaatgggtctcaacaatctccggggggggcacttacatctattac
cccgactcagtcaaggggagatttaccatttcacgagacaacgctaagaataccctgtatttgcagatgaattctctgaga
gcagaggacacagctgtttactattgtacccgcgacaactatggcaggaactacgactacggtatggactattggggacaa
gggacattggttacagtgagcagtggcggcgggggcagcggaggaggaggcagcggtggcggaggcagcgagatagtgctc
acgcagtcacccgcgactctcagtctctcacctggggaacgagctaccctgacgtgctctgctacctcctcagtgtcatat
attcactggtatcagcaacggcccgggcagtcccctagattgctcatttatagtacctctaatctggcctcaggtatccct
gcacgattttctggatctggttcaggttctgattacaccctcactatctctagcctggagcctgaagactttgccgtttat
tactgccagcagaggtctagctccccattcacctttgggagtgggaccaaggttgaaattaaagagtctccaaaggcacag
gcctcctcagtgcccactgcacaaccccaagcagagggcagcctcgccaaggcaaccacagccccagccaccacccgtaac
acaggaagaggcggcgaagagaagaaaaaggagaaggagaaagaggaacaagaagagagagagacaaagacaccagcacct
gaactcctggggggaccgtcagtcttcctcttccccccaaaacccaaggacaccctcatgatctcccggacccctgaggtc
acatgcgtggtggtggacgtgagccacgaagaccctgaggtcaagttcaactggtacgtggacggcgtggaggtgcataat
gccaagacaaagccgcgggaggagcagtacaacagcacgtaccgtgtggtcagcgtcctcaccgtcctgcaccaggactgg
ctgaatggcaaggagtacaagtgcaaggtctccaacaaagccctcccagcccccatcgagaaaaccatctccaaagccaaa
gggcagccccgagaaccacaggtgtacaccctgcccccatcccgggaggagatgaccaagaaccaggtcagcctgacctgc
ctggtcaaaggcttctatcccagcgacatcgccgtggagtgggagagcaatgggcagccggagaacaactacaagaccacg
cctcccgtgctggactccgacggctccttcttcctcaggagcaagctcaccgtggacaagagcaggtggcagcaggggaac
gtcttctcatgctccgtgatgcatgaggctctgcacaaccactacacgcagaagagcctctccctgtctccgggtaaaatc
tacatctgggcgcccttggccgggacttgtggggtccttctcctgtcactggttatcaccctttactgcaaacggggcaga
aagaaactcctgtatatattcaaacaaccatttatgagaccagtacaaactactcaagaggaagatggctgtagctgccga
tttccagaagaagaagaaggaggatgtgaactgagagtgaagttcagcaggagcgcagacgcccccgcgtacaagcagggc
cagaaccagctctataacgagctcaatctaggacgaagagaggagtacgatgttttggacaagagacgtggccgggaccct
gagatggggggaaagccgagaaggaagaaccctcaggaaggcctgtacaatgaactgcagaaagataagatggcggaggcc
tacagtgagattgggatgaaaggcgagcgccggaggggcaaggggcacgatggcctttaccagggtctcagtacagccacc
aaggacacctacgacgcccttcacatgcaggccctgccccctcgctgataa
(amino acids)
(SEQ ID NO: 773)
MALPVTALLLPLALLLHAARPEVQLVESGGGLVKPGGSLRLSCAASGFTFSRYGMSWVRQAPGKRLEWVSTISGGGTYIY
YPDSVKGRFTISRDNAKNTLYLQMNSLRAEDTAVYYCTRDNYGRNYDYGMDYWGQGTLVTVSSGGGGSGGGGSGGGGSEI
VLTQSPATLSLSPGERATLTCSATSSVSYIHWYQQRPGQSPRLLIYSTSNLASGIPARFSGSGSGSDYTLTISSLEPEDF
AVYYCQQRSSSPFTFGSGTKVEIKESPKAQASSVPTAQPQAEGSLAKATTAPATTRNTGRGGEEKKKEKEKEEQEERETK
TPAPELLGGPSVFLFPPKPKDTLMISRTPEVTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREEMTKNQVSLTCLVKGFYPSDIAVEWESNGQPE
NNYKTTPPVLDSDGSFFLRSKLTVDKSRWQQGNVFSCSVMHEALHNHYTQKSLSLSPGKIYIWAPLAGTCGVLLLSLVIT
LYCKRGRKKLLYIFKQPFMRPVQTTQEEDGCSCRFPEEEEGGCELRVKFSRSADAPAYKQGQNQLYNELNLGRREEYDVL
DKRRGRDPEMGGKPRRKNPQEGLYNELQKDKMAEAYSEIGMKGERRRGKGHDGLYQGLSTATKDTYDALHMQALPPR**
NFATc1P2-MMP9
(DNA)
(SEQ ID NO: 774)
caggcctggggacactcgcggcgggaagatttggaggggaggggagggggaggggcgtgggggcgcggcctcgctggagt
ccccctgaccccccgacccccgcccaccggcctgggcgtcctcccgcggcccctcctcccctcccggcgcccggtgctct
ggggcgcgtgccacgcctggctcggcgccgtaggggcccccgcaggtagagacccctggaaatggcctcgacgccgcagg
agcgaggcggccaccaccccgctaatccgggcacgtctctccaggccgaggcctgcggtggaaaagccggggttccattt
gtgctgagtcggggcggccgaatggagccaggcctcgggacgcgggacggacgggctctggccgcgcaccttcgcgggct
ctgcagcgcccgaccgcctcccccggcagggaggaggcgcttgtggggggcacccacggggcacagtgatccctgggggt
ctgcggacctcctgggccccgcagcagacacgagtttagcctttgggtttagtttaaatcacataagggtgtcgtgcaat
cgatttatggtttctacacaccagacactttaacctccaaccccccccatccaaagccaacaagaaaatgcggtgccgtg
ttggcagctgagctgcgcccgaagagacgcagggagacgtaagagaggaaagtgtgagtggccggggggcctccccccgt
cagaagtcgcgcagtcgcgcccataaaacgccccctccgggcggctagggcaggtgagcgcgtccccgggcctccccacg
ccggcccctgccacaggccgtctaggtcgagcagatatttacagaataaaaatgacaataactcgacgtcccgggacggc
cacgcaatctgttagtaatttagcgggatgggaatttcctttctagggcctgccagtgaagcgcttttccaaatttccac
agcgggggaagcctgcgattttacataatgacttcagcatgccgggctttctcgacacccctccccggcccccggccccc
gccccccgccccttttccagcagggccgggctccctccggacacccgcgtggactcaggcgtcccgtctggcccgttcgc
ccccgtttcccccgccagccccagcgcccccctgcccggcccccggattccccgttcccgcccctacgcccccatcccct
ccccgtgcgcccctccccgtgcgcccccctccccgtgcgccccccctccccgtgcgcccccctccccgtgcgccccccct
ccccgggcgcccccctccccgggcgccccccctccccgtgcgcccccccctccccgtgcgccccccctccccgtgcgcgc
cccgcctcttgcgcccctgcccccaggcgagcggctgccgcggcgcggggaggggcgggcgctcggcgactcgtccccgg
ggccccgcgcgggcccgggcagcaggggcgtgatgtcacggcagggagggggcgcgggagccgccgggccggcggggagg
cgggggaggtgttttccagctttaaaaaggcaggaggcagagcgcggccctgcgtcagagcgagactcagaggctccgaa
ctcgccggcggagtcgccgcgccagatcccagcagcagggcgcggaagcttctctcgacattcgtttctagagccaccat
gagcctctggcagcccctggtcctggtgctcctggtgctgggctgctgctttgctgcccccagacagcgccagtccaccct
tgtgctcttccctggagacctgagaaccaatctcaccgacaggcagctggcagaggaatacctgtaccgctatggttacac
tcgggtggcagagatgcgtggagagtcgaaatctctggggcctgcgctgctgcttctccagaagcaactgtccctgcccga
gaccggtgagctggatagcgccacgctgaaggccatgcgaaccccacggtgcggggtcccagacctgggcagattccaaac
ctttgagggcgacctcaagtggcaccaccacaacatcacctattggatccaaaactactcggaagacttgccgcgggcggt
gattgacgacgcctttgcccgcgccttcgcactgtggagcgcggtgacgccgctcaccttcactcgcgtgtacagccggga
cgcagacatcgtcatccagtttggtgtcgcggagcacggagacgggtatcccttcgacgggaaggacgggctcctggcaca
cgcctttcctcctggccccggcattcagggagacgcccatttcgacgatgacgagttgtggtccctgggcaagggcgtcgt
ggttccaactcggtttggaaacgcagatggcgcggcctgccacttccccttcatcttcgagggccgctcctactctgcctg
caccaccgacggtcgctccgacggcttgccctggtgcagtaccacggccaactacgacaccgacgaccggtttggcttctg
ccccagcgagagactctacacccaggacggcaatgctgatgggaaaccctgccagtttccattcatcttccaaggccaatc
ctactccgcctgcaccacggacggtcgctccgacggctaccgctggtgcgccaccaccgccaactacgaccgggacaagct
cttcggcttctgcccgacccgagctgactcgacggtgatggggggcaactcggcgggggagctgtgcgtcttccccttcac
tttcctgggtaaggagtactcgacctgtaccagcgagggccgcggagatgggcgcctctggtgcgctaccacctcgaactt
tgacagcgacaagaagtggggcttctgcccggaccaaggatacagtttgttcctcgtggcggcgcatgagttcggccacgc
gctgggcttagatcattcctcagtgccggaggcgctcatgtaccctatgtaccgcttcactgaggggccccccttgcataa
ggacgacgtgaatggcatccggcacctctatggtcctcgccctgaacctgagccacggcctccaaccaccaccacaccgca
gcccacggctcccccgacggtctgccccaccggaccccccactgtccacccctcagagcgccccacagctggccccacagg
tcccccctcagctggccccacaggtccccccactgctggcccttctacggccactactgtgcctttgagtccggtggacga
tgcctgcaacgtgaacatcttcgacgccatcgcggagattgggaaccagctgtatttgttcaaggatgggaagtactggcg
attctctgagggcagggggagccggccgcagggccccttccttatcgccgacaagtggcccgcgctgccccgcaagctgga
ctcggtctttgaggagcggctctccaagaagcttttcttcttctctgggcgccaggtgtgggtgtacacaggcgcgtcggt
gctgggcccgaggcgtctggacaagctgggcctgggagccgacgtggcccaggtgaccggggccctccggagtggcagggg
gaagatgctgctgttcagcgggcggcgcctctggaggttcgacgtgaaggcgcagatggtggatccccggagcgccagcga
ggtggaccggatgttccccggggtgcctttggacacgcacgacgtcttccagtaccgagagaaagcctatttctgccagga
ccgcttctactggcgcgtgagttcccggagtgagttgaaccaggtggaccaagtgggctacgtgacctatgacatcctgca
gtgccctgaggacgattacaaggatgacgacgataagtgataa
NFATc1P2-MMP9cat
(DNA)
(SEQ ID NO: 775)
caggcctggggacactcgcggcgggaagatttggaggggaggggagggggaggggcgtgggggcgcggcctcgctggagt
ccccctgaccccccgacccccgcccaccggcctgggcgtcctcccgcggcccctcctcccctcccggcgcccggtgctct
ggggcgcgtgccacgcctggctcggcgccgtaggggcccccgcaggtagagacccctggaaatggcctcgacgccgcagg
agcgaggcggccaccaccccgctaatccgggcacgtctctccaggccgaggcctgcggtggaaaagccggggttccattt
gtgctgagtcggggcggccgaatggagccaggcctcgggacgcgggacggacgggctctggccgcgcaccttcgcgggct
ctgcagcgcccgaccgcctcccccggcagggaggaggcgcttgtggggggcacccacggggcacagtgatccctgggggt
ctgcggacctcctgggccccgcagcagacacgagtttagcctttgggtttagtttaaatcacataagggtgtcgtgcaat
cgatttatggtttctacacaccagacactttaacctccaaccccccccatccaaagccaacaagaaaatgcggtgccgtg
ttggcagctgagctgcgcccgaagagacgcagggagacgtaagagaggaaagtgtgagtggccggggggcctccccccgt
cagaagtcgcgcagtcgcgcccataaaacgccccctccgggcggctagggcaggtgagcgcgtccccgggcctccccacg
ccggcccctgccacaggccgtctaggtcgagcagatatttacagaataaaaatgacaataactcgacgtcccgggacggc
cacgcaatctgttagtaatttagcgggatgggaatttcctttctagggcctgccagtgaagcgcttttccaaatttccac
agcgggggaagcctgcgattttacataatgacttcagcatgccgggctttctcgacacccctccccggcccccggccccc
gccccccgccccttttccagcagggccgggctccctccggacacccgcgtggactcaggcgtcccgtctggcccgttcgc
ccccgtttcccccgccagccccagcgcccccctgcccggcccccggattccccgttcccgcccctacgcccccatcccct
ccccgtgcgcccctccccgtgcgcccccctccccgtgcgccccccctccccgtgcgcccccctccccgtgcgccccccct
ccccgggcgcccccctccccgggcgccccccctccccgtgcgcccccccctccccgtgcgccccccctccccgtgcgcgc
cccgcctcttgcgcccctgcccccaggcgagcggctgccgcggcgcggggaggggcgggcgctcggcgactcgtccccgg
ggccccgcgcgggcccgggcagcaggggcgtgatgtcacggcagggagggggcgcgggagccgccgggccggcggggagg
cgggggaggtgttttccagctttaaaaaggcaggaggcagagcgcggccctgcgtcagagcgagactcagaggctccgaa
ctcgccggcggagtcgccgcgccagatcccagcagcagggcgcggaagcttctctcgacattcgtttctagagccaccat
gagcctctggcagcccctggtcctggtgctcctggtgctgggctgctgctttgctttccaaacctttgagggcgacctcaa
gtggcaccaccacaacatcacctattggatccaaaactactcggaagacttgccgcgggcggtgattgacgacgcctttgc
ccgcgccttcgcactgtggagcgcggtgacgccgctcaccttcactcgcgtgtacagccgggacgcagacatcgtcatcca
gtttggtgtcgcggagcacggagacgggtatcccttcgacgggaaggacgggctcctggcacacgcctttcctcctggccc
cggcattcagggagacgcccatttcgacgatgacgagttgtggtccctgggcaagggcgtcgtggttccaactcggtttgg
aaacgcagatggcgcggcctgccacttccccttcatcttcgagggccgctcctactctgcctgcaccaccgacggtcgctc
cgacggcttgccctggtgcagtaccacggccaactacgacaccgacgaccggtttggcttctgccccagcgagagactcta
cacccaggacggcaatgctgatgggaaaccctgccagtttccattcatcttccaaggccaatcctactccgcctgcaccac
ggacggtcgctccgacggctaccgctggtgcgccaccaccgccaactacgaccgggacaagctcttcggcttctgcccgac
ccgagctgactcgacggtgatggggggcaactcggcgggggagctgtgcgtcttccccttcactttcctgggtaaggagta
ctcgacctgtaccagcgagggccgcggagatgggcgcctctggtgcgctaccacctcgaactttgacagcgacaagaagtg
gggcttctgcccggaccaaggatacagtttgttcctcgtggcggcgcatgagttcggccacgcgctgggcttagatcattc
ctcagtgccggaggcgctcatgtaccctatgtaccgcttcactgaggggccccccttgcataaggacgacgtgaatggcat
ccggcacctctatggtcctcgccctgaacctgattacaaggatgacgacgataagtgataa
NFAT response element 2
(DNA)
(SEQ ID NO: 776)
aagaggaaaatttgtttcatacagaaggcgtt
NFAT response element 2 repeats
(DNA)
(SEQ ID NO: 777)
aagaggaaaatttgtttcatacagaaggcgttaagaggaaaatttgtttcatacagaaggcgttaagaggaaaatttgttt
catacagaaggcgttaagaggaaaatttgtttcatacagaaggcgtt
CMV minimal promoter 2
(DNA)
(SEQ ID NO: 778)
taggcgtgtacggtgggaggcctatataagcagagctcgtttagtgaaccgtcagatcgcctggagacgccatccacgctg
ttttgacctccatagaagacaccgggaccgatccagc
NFATRE2mCMV2-MMP9
(DNA)
(SEQ ID NO: 779)
aagaggaaaatttgtttcatacagaaggcgttaagaggaaaatttgtttcatacagaaggcgttaagaggaaaatttgttt
catacagaaggcgttaagaggaaaatttgtttcatacagaaggcgttactagttaggcgtgtacggtgggaggcctatata
agcagagctcgtttagtgaaccgtcagatcgcctggagacgccatccacgctgttttgacctccatagaagacaccgggac
cgatccagcctctcgacattcgtttctagagccaccatgagcctctggcagcccctggtcctggtgctcctggtgctgggc
tgctgctttgctgcccccagacagcgccagtccacccttgtgctcttccctggagacctgagaaccaatctcaccgacagg
cagctggcagaggaatacctgtaccgctatggttacactcgggtggcagagatgcgtggagagtcgaaatctctggggcct
gcgctgctgcttctccagaagcaactgtccctgcccgagaccggtgagctggatagcgccacgctgaaggccatgcgaacc
ccacggtgcggggtcccagacctgggcagattccaaacctttgagggcgacctcaagtggcaccaccacaacatcacctat
tggatccaaaactactcggaagacttgccgcgggcggtgattgacgacgcctttgcccgcgccttcgcactgtggagcgcg
gtgacgccgctcaccttcactcgcgtgtacagccgggacgcagacatcgtcatccagtttggtgtcgcggagcacggagac
gggtatcccttcgacgggaaggacgggctcctggcacacgcctttcctcctggccccggcattcagggagacgcccatttc
gacgatgacgagttgtggtccctgggcaagggcgtcgtggttccaactcggtttggaaacgcagatggcgcggcctgccac
ttccccttcatcttcgagggccgctcctactctgcctgcaccaccgacggtcgctccgacggcttgccctggtgcagtacc
acggccaactacgacaccgacgaccggtttggcttctgccccagcgagagactctacacccaggacggcaatgctgatggg
aaaccctgccagtttccattcatcttccaaggccaatcctactccgcctgcaccacggacggtcgctccgacggctaccgc
tggtgcgccaccaccgccaactacgaccgggacaagctcttcggcttctgcccgacccgagctgactcgacggtgatgggg
ggcaactcggcgggggagctgtgcgtcttccccttcactttcctgggtaaggagtactcgacctgtaccagcgagggccgc
ggagatgggcgcctctggtgcgctaccacctcgaactttgacagcgacaagaagtggggcttctgcccggaccaaggatac
agtttgttcctcgtggcggcgcatgagttcggccacgcgctgggcttagatcattcctcagtgccggaggcgctcatgtac
cctatgtaccgcttcactgaggggccccccttgcataaggacgacgtgaatggcatccggcacctctatggtcctcgccct
gaacctgagccacggcctccaaccaccaccacaccgcagcccacggctcccccgacggtctgccccaccggaccccccact
gtccacccctcagagcgccccacagctggccccacaggtcccccctcagctggccccacaggtccccccactgctggccct
tctacggccactactgtgcctttgagtccggtggacgatgcctgcaacgtgaacatcttcgacgccatcgcggagattggg
aaccagctgtatttgttcaaggatgggaagtactggcgattctctgagggcagggggagccggccgcagggccccttcctt
atcgccgacaagtggcccgcgctgccccgcaagctggactcggtctttgaggagcggctctccaagaagcttttcttcttc
tctgggcgccaggtgtgggtgtacacaggcgcgtcggtgctgggcccgaggcgtctggacaagctgggcctgggagccgac
gtggcccaggtgaccggggccctccggagtggcagggggaagatgctgctgttcagcgggcggcgcctctggaggttcgac
gtgaaggcgcagatggtggatccccggagcgccagcgaggtggaccggatgttccccggggtgcctttggacacgcacgac
gtcttccagtaccgagagaaagcctatttctgccaggaccgcttctactggcgcgtgagttcccggagtgagttgaaccag
gtggaccaagtgggctacgtgacctatgacatcctgcagtgccctgaggacgattacaaggatgacgacgataagtgataa
NFATRE2mCMV2-MMP9cat
(DNA)
(SEQ ID NO: 780)
aagaggaaaatttgtttcatacagaaggcgttaagaggaaaatttgtttcatacagaaggcgttaagaggaaaatttgttt
catacagaaggcgttaagaggaaaatttgtttcatacagaaggcgttactagttaggcgtgtacggtgggaggcctatata
agcagagctcgtttagtgaaccgtcagatcgcctggagacgccatccacgctgttttgacctccatagaagacaccgggac
cgatccagcctcgagctctcgacattcgtttctagagccaccatgagcctctggcagcccctggtcctggtgctcctggtg
ctgggctgctgctttgctttccaaacctttgagggcgacctcaagtggcaccaccacaacatcacctattggatccaaaac
tactcggaagacttgccgcgggcggtgattgacgacgcctttgcccgcgccttcgcactgtggagcgcggtgacgccgctc
accttcactcgcgtgtacagccgggacgcagacatcgtcatccagtttggtgtcgcggagcacggagacgggtatcccttc
gacgggaaggacgggctcctggcacacgcctttcctcctggccccggcattcagggagacgcccatttcgacgatgacgag
ttgtggtccctgggcaagggcgtcgtggttccaactcggtttggaaacgcagatggcgcggcctgccacttccccttcatc
ttcgagggccgctcctactctgcctgcaccaccgacggtcgctccgacggcttgccctggtgcagtaccacggccaactac
gacaccgacgaccggtttggcttctgccccagcgagagactctacacccaggacggcaatgctgatgggaaaccctgccag
tttccattcatcttccaaggccaatcctactccgcctgcaccacggacggtcgctccgacggctaccgctggtgcgccacc
accgccaactacgaccgggacaagctcttcggcttctgcccgacccgagctgactcgacggtgatggggggcaactcggcg
ggggagctgtgcgtcttccccttcactttcctgggtaaggagtactcgacctgtaccagcgagggccgcggagatgggcgc
ctctggtgcgctaccacctcgaactttgacagcgacaagaagtggggcttctgcccggaccaaggatacagtttgttcctc
gtggcggcgcatgagttcggccacgcgctgggcttagatcattcctcagtgccggaggcgctcatgtaccctatgtaccgc
ttcactgaggggccccccttgcataaggacgacgtgaatggcatccggcacctctatggtcctcgccctgaacctgattac
aaggatgacgacgataagtgataa
NFATc1 Promoter fragment (P1)
(DNA)
(SEQ ID NO: 781)
aggcaggaggaagaggaaaggggcgcagggcgctcggggagcagagccgggggcccgcggtggccgcagaggccgggccgg
ggcgcagaggccgggcgagctggccgcgctctgggccgccgcctccggaactccctgcgcctggcgcgcggccaccgtgg
tcccggcaacggcattaaacagagggaaacagacccgggattccgtcacccgggcggggggataaggacggctttgagag
cagacaggaaaagggagcttttctgcatggggtgaaaaaattatttattgaaggaggaggaggcggcagcggaggaaggg
gaggggcgggaggaggaggaagagccggccgcccccgccccggccccggctcctcaggagccaagggcagcctcgccagg
tcggtcccgggctcgaggaccgcggctggggtcgaggggctcagtctcccacgtgaccggctgggcgcgccccgccagac
ccggcctcgggattccctcctcccggcgagtctccgcccgccccgtcctggaggtggggagaaggagggcggggcggggg
ggacggaaactctccccgccaaatcctggccccaggcctggggacactcgcggcgggaagatttggaggggaggggaggg
ggaggggcgtgggggcgcggcctcgctggagtccccctgaccccccgacccccgcccaccggcctgggcgtcctcccgcg
gcccctcctcccctcccggcgcccggtgctctggggcgcgtgccacgcctggctcggcgccgtaggggcccccgcaggta
gagacccctggaaatggcctcgacgccgcaggagcgaggcggccaccaccccgctaatccgggcacgtctctccaggccg
aggcctgcggtggaaaagccggggttccatttgtgctgagtcggggcggccgaatggagccaggcctcgggacgcgggac
ggacgggctctggccgcgcaccttcgcgggctctgcagcgcccgaccgcctcccccggcagggaggaggcgcttgtgggg
ggcacccacggggcacagtgatccctgggggtctgcggacctcctgggccccgcagcagacacgagtttagcctttgggt
ttagtttaaatcacataagggtgtcgtgcaatcgatttatggtttctacacaccagacactttaacctccaacccccccc
atccaagccaacaagaaaatgcggtgccgtgttggcagctgagctgcgcccgaagagacgcagggagacgtaagagagga
aagtgtgagtggccggggggcctccccccgtcagaagtcgcgcagtcgcgcccataaaacgccccctccgggcggctagg
gcaggtgagcgcgtccccgggcctccccacgccggcccctgccacagagccgtctaggtcgagcagatatttacagaata
aaaatgacaataactcgacgtcccgggacggccacgcaatctgttagtaatttagcgggatgggaatttcctttctaggg
cctgccagtgaagcgcttttccaaatttccacagcgggggaagcctgcgattttacataatgacttcagcatgccgggct
ttctcgacacccctccccggcccccggcccccgccccccgccccttttccagcagggccgggctccctccggacacccgc
gtggactcaggcgtcccgtctggcccgttcgcccccgtttcccccgccagccccagcgcccccctgcccggcccccggat
tccccgttcccgcccctacgcccccatcccctccccgtgcgcccctccccgtgcgcccccctccccgtgcgccccccctc
cccgtgcgcccccctccccgtgcgccccccctccccgggcgcccccctccccgggcgccccccctccccgtgcgcccccc
cctccccgtgcgccccccctccccgtgcgcgccccgcctcttgcgcccctgcccccaggcgagcggctgccgcggcgcgg
ggaggggcgggcgctcggcgactcgtccccggggccccgcgcgggcccgggcagcaggggcgtgatgtcacggcagggag
ggggcgcgggagccgccgggccggcggggaggcgggggaggtgttttccagctttaaaaaggcaggaggcagagcgcggc
cctgcgtcagagcgagactcagaggctccgaactcgccggcggagtcgccgcgccagatcccagcagcagggcgcgg
NFATc1 Promoter fragment (P2)
(DNA)
(SEQ ID NO: 782)
aggcaggaggaagaggaaaggggcgcagggcgctcggggagcagagccgggggcccgcggtggccgcagaggccgggccgg
ggcgcagaggccgggcgagctggccgcgctctgggccgccgcctccggaactccctgcgcctggcgcgcggccaccgtgg
tcccggcaacggcattaaacagagggaaacagacccgggattccgtcacccgggcggggggataaggacggctttgagag
cagacaggaaaagggagcttttctgcatggggtgaaaaaattatttattgaaggaggaggaggcggcagcggaggaaggg
gaggggcgggaggaggaggaagagccggccgcccccgccccggccccggctcctcaggagccaagggcagcctcgccagg
tcggtcccgggctcgaggaccgcggctggggtcgaggggctcagtctcccacgtgaccggctgggcgcgccccgccagac
ccggcctcgggattccctcctcccggcgagtctccgcccgccccgtcctggaggtggggagaaggagggcggggcggggg
ggacggaaactctccccgccaaatcctggccccaggcctggggacactcgcggcgggaagatttggaggggaggggaggg
ggaggggcgtgggggcgcggcctcgctggagtccccctgaccccccgacccccgcccaccggcctgggcgtcctcccgcg
gcccctcctcccctcccggcgcccggtgctctggggcgcgtgccacgcctggctcggcgccgtaggggcccccgcaggta
gagacccctggaaatggcctcgacgccgcaggagcgaggcggccaccaccccgctaatccgggcacgtctctccaggccg
aggcctgcggtggaaaagccggggttccatttgtgctgagtcggggcggccgaatggagccaggcctcgggacgcgggac
ggacgggctctggccgcgcaccttcgcgggctctgcagcgcccgaccgcctcccccggcagggaggaggcgcttgtgggg
ggcacccacggggcacagtgatccctgggggtctgcggacctcctgggccccgcagcagacacgagtttagcctttgggt
ttagtttaaatcacataagggtgtcgtgcaatcgatttatggtttctacacaccagacactttaacctccaacccccccc
atccaagccaacaagaaaatgcggtgccgtgttggcagctgagctgcgcccgaagagacgcagggagacgtaagagagga
aagtgtgagtggccggggggcctccccccgtcagaagtcgcgcagtcgcgcccataaaacgccccctccgggcggctagg
gcaggtgagcgcgtccccgggcctccccacgccggcccctgccacagagccgtctaggtcgagcagatatttacagaata
aaaatgacaataactcgacgtcccgggacggccacgcaatctgttagtaatttagcgggatgggaatttcctttctaggg
cctgccagtgaagcgcttttccaaatttccacagcgggggaagcctgcgattttacataatgacttcagcatgccgggct
ttctcgacacccctccccggcccccggcccccgccccccgccccttttccagcagggccgggctccctccggacacccgc
gtggactcaggcgtcccgtctggcccgttcgcccccgtttcccccgccagccccagcgcccccctgcccggcccccggat
tccccgttcccgcccctacgcccccatcccctccccgtgcgcccctccccgtgcgcccccctccccgtgcgccccccctc
cccgtgcgcccccctccccgtgcgccccccctccccgggcgcccccctccccgggcgccccccctccccgtgcgcccccc
cctccccgtgcgccccccctccccgtgcgcgccccgcctcttgcgcccctgcccccaggcgagcggctgccgcggcgcgg
ggaggggcgggcgctcggcgactcgtccccggggccccgcgcgggcccgggcagcaggggcgtgatgtcacggcagggag
ggggcgcgggagccgccgggccggcggggaggcgggggaggtgttttccagctttaaaaaggcaggaggcagagcgcggc
cctgcgtcagagcgagactcagagg
NFATc1 Promoter fragment (P3)
(DNA)
(SEQ ID NO: 783)
caggcctggggacactcgcggcgggaagatttggaggggaggggagggggaggggcgtgggggcgcggcctcgctggagt
ccccctgaccccccgacccccgcccaccggcctgggcgtcctcccgcggcccctcctcccctcccggcgcccggtgctct
ggggcgcgtgccacgcctggctcggcgccgtaggggcccccgcaggtagagacccctggaaatggcctcgacgccgcagg
agcgaggcggccaccaccccgctaatccgggcacgtctctccaggccgaggcctgcggtggaaaagccggggttccattt
gtgctgagtcggggcggccgaatggagccaggcctcgggacgcgggacggacgggctctggccgcgcaccttcgcgggct
ctgcagcgcccgaccgcctcccccggcagggaggaggcgcttgtggggggcacccacggggcacagtgatccctgggggt
ctgcggacctcctgggccccgcagcagacacgagtttagcctttgggtttagtttaaatcacataagggtgtcgtgcaat
cgatttatggtttctacacaccagacactttaacctccaaccccccccatccaaagccaacaagaaaatgcggtgccgtg
ttggcagctgagctgcgcccgaagagacgcagggagacgtaagagaggaaagtgtgagtggccggggggcctccccccgt
cagaagtcgcgcagtcgcgcccataaaacgccccctccgggcggctagggcaggtgagcgcgtccccgggcctccccacg
ccggcccctgccacaggccgtctaggtcgagcagatatttacagaataaaaatgacaataactcgacgtcccgggacggc
cacgcaatctgttagtaatttagcgggatgggaatttcctttctagggcctgccagtgaagcgcttttccaaatttccac
agcgggggaagcctgcgattttacataatgacttcagcatgccgggctttctcgacacccctccccggcccccggccccc
gccccccgccccttttccagcagggccgggctccctccggacacccgcgtggactcaggcgtcccgtctggcccgttcgc
ccccgtttcccccgccagccccagcgcccccctgcccggcccccggattccccgttcccgcccctacgcccccatcccct
ccccgtgcgcccctccccgtgcgcccccctccccgtgcgccccccctccccgtgcgcccccctccccgtgcgccccccct
ccccgggcgcccccctccccgggcgccccccctccccgtgcgcccccccctccccgtgcgccccccctccccgtgcgcgc
cccgcctcttgcgcccctgcccccaggcgagcggctgccgcggcgcggggaggggcgggcgctcggcgactcgtccccgg
ggccccgcgcgggcccgggcagcaggggcgtgatgtcacggcagggagggggcgcgggagccgccgggccggcggggagg
cgggggaggtgttttccagctttaaaaaggcaggaggcagagcgcggccctgcgtcagagcgagactcagaggctccgaa
ctcgccggcggagtcgccgcgccagatcccagcagcagggcgcgg
pNFAT-MMP9cat-1 gBLOCK sequence
(DNA)
(SEQ ID NO: 784)
aagaggaaaatttgtttcatacagaaggcgttactagttaggcgtgtacggtgggaggcctatataagcagagctcgttta
gtgaaccgtcagatcgcctggagacgccatccacgctgttttgacctccatagaagacaccgggaccgatccagcctctcg
acattcgtttctagagccaccatgagcctctggcagcccctggtcctggtgctcctggtgctgggctgctgctttgctttc
caaacctttgagggcgacctcaagtggcaccaccacaacatcacctattggatccaaaactactcggaagacttgccgcgg
gcggtgattgacgacgcctttgcccgcgccttcgcactgtggagcgcggtgacgccgctcaccttcactcgcgtgtacagc
cgggacgcagacatcgtcatccagtttggtgtcgcggagcacggagacgggtatcccttcgacgggaaggacgggctcctg
gcacacgcctttcctcctggccccggcattcagggagacgcccatttcgacgatgacgagttgtggtccctgggcaagggc
gtcgtggttccaactcggtttggaaacgcagatggcgcggcctgccacttccccttcatcttcgagggccgctcctactct
gcctgcaccaccgacggtcgctccgacggcttgccctggtgcagtaccacggccaactacgacaccgacgaccggtttggc
ttctgccccagcgagagactctacacccaggacggcaatgctgatgggaaaccctgccagtttccattcatcttccaaggc
caatcctactccgcctgcaccacggacggtcgctccgacggctaccgctggtgcgccaccaccgccaactacgaccgggac
aagctcttcggcttctgcccgacccgagctgactcg
pNFAT-MMP9cat-2 gBLOCK sequence:
(DNA)
(SEQ ID NO: 785)
ttcggcttctgcccgacccgagctgactcgacggtgatggggggcaactcggcgggggagctgtgcgtcttccccttcact
ttcctgggtaaggagtactcgacctgtaccagcgagggccgcggagatgggcgcctctggtgcgctaccacctcgaacttt
gacagcgacaagaagtggggcttctgcccggaccaaggatacagtttgttcctcgtggcggcgcatgagttcggccacgcg
ctgggcttagatcattcctcagtgccggaggcgctcatgtaccctatgtaccgcttcactgaggggccccccttgcataag
gacgacgtgaatggcatccggcacctctatggtcctcgccctgaacctgattacaaggatgacgacgataagtgataagct
agctcgactcgacaatcaacctctggattacaaaatttgtgaaagattgactggtattcttaactatgttgctccttttac
gctatgtggatacgctgctttaatgcctttgtatcatgctattgcttcccgtatggctttcattttctcctccttgtataa
atcctggttgctgtctctttatgaggagttgtggcccgttgtcaggcaacgtggcgtggtgtgcactgtgtttgctgacgc
aacccccactggttggggcattgccaccacctgtcagctcctttccgggactttcgctttccccctccctattgccacggc
ggaactcatcgccgcctgccttgcccgctgctggacaggggctcggctgttgggcactgacaattccgtggtgttgtcggg
gaaatcatcgtcctttccttggctgctcgcctgtgttgccacctggattctgcgcgggacgtccttctgctacgtcccttc
ggccctcaatccagcggaccttccttcccgcggcctgctgccggctctgcggcctcttccgcatcttcgccttcgccctca
gacgagtcggatctccctttgggccgcctccccgcctggaattaattcgagctcggtacctttaagaccaatgacttacaa
ggcagctgtag
Primer
(DNA)
(SEQ ID NO: 786)
tagatggtaccaagaggaaaatttgtttcatacag
Primer
(DNA)
(SEQ ID NO: 787)
tagataagcttgctggatcggtcccggtgtc
Primer
(DNA)
(SEQ ID NO: 788)
tcatacagaaggcgttactagttaggcgtgtacggtgg
Primer
(DNA)
(SEQ ID NO: 789)
acagtaccggattgccaagcttttatcacttatcgtcgtcatccttg
Primer
(DNA)
(SEQ ID NO: 790)
aagttggtaccgttccaaacctttgagggcgacc
Primer
(DNA)
(SEQ ID NO: 791)
aagttctcgagcaggttcagggcgaggaccatag
Primer
(DNA)
(SEQ ID NO: 792)
attgactcgagctctcgacattcgtttctagagc
Primer
(DNA)
(SEQ ID NO: 793)
attgaaagcttttatcacttatcgtcgtcatccttg
Primer
(DNA)
(SEQ ID NO: 794)
tagcaaaataggctgtccc
Primer
(DNA)
(SEQ ID NO: 795)
attgactcgaggctggatcggtcccggtgtc
Primer
(DNA)
(SEQ ID NO: 796)
aagacaccgggaccgatccagcctcgagagacccaagctggctagccacc
Primer
(DNA)
(SEQ ID NO: 797)
ttaccaacagtaccggattgccaagcttttatcacttatcgtcgtcatcc
Primer
(DNA)
(SEQ ID NO: 798)
attgaaagcttctctcgacattcgtttctagagc
Primer
(DNA)
(SEQ ID NO: 799)
attgagagctcttatcacttatcgtcgtcatc
NFAT modif 1 gBLOCK sequence:
(DNA)
(SEQ ID NO: 800)
attctgtggataaccgtattaccgctagcatggatctcggggacgtctaactactaagcgagagtagggaactgccaggc
atcaaataaaacgaaaggctcagtcggaagactgggcctttcgttttatctgttgtttgtcggtgaacgctctcctgagt
aggacaaatccgccgggagcggatttgaacgttgtgaagcaacggcccggagggtggcgggcaggacgcccgccataaac
tgccaggcatcaaactaagcagaaggccatcctgacggatggcctttttgcgtttctacaaactcttcctgttagttagt
tacttaagctcgggccccaaattatgattttgttctgactgatagtgacctgttcgttgcaacaaattgataagcaatgc
ttttttataatgccaactttgtacaaaaaagcaggcttcgctgtgccttctagttgccagccatctgttgtttgcccctc
ccccgtgccttccttgaccctggaaggtgccactcccactgtcctttcctaataaaatgaggaaattgcatcgcattgtc
tgagtaggtgtcattctattctggggggtggggtggggcaggacagcaagggggaggattgggaagacaatagcaggcat
gctggggatgcggtgggctctatggttcgaaggagatagaaccagatcttgactagtggtaccgaattccaggcctgggg
acactcgcggcgggaa
NFAT modif 2 gBLOCK sequences:
(DNA)
(SEQ ID NO: 801)
acaaggatgacgacgataagtgataagagctcgctagcgatatcgccaccatgggggtaaaagttcttttcgcgcttatct
gtatcgcggttgcagaagctaaaccaacagaaaataatgaagactttaacattgttgccgtggcatcgaacttcgccacaa
ccgatttggacgctgatcgcgggaaactgcccggcagccaccacggctcgcaaagctcggtttgcccggacttgcaccgtt
gtgggcgcgggcaagcgcatcgtgagcagcgtcgcttgcagcaccgtgggcgcggccagcaattacgcgaccacggaagcc
gttgttaaccacgcgaagtagcacgtcaagaagctgccgctggaggtgctcaaagagctggaagccaatgcccggaaagct
ggctgcaccaggggctgtctgatctgcctgtcccacatcaagtgcacgcccaagatgaagaagttcatcccaggacgctgc
cacacctacgaaggcgacaaagagtccgcacagggcggcataggcgaggcgatcgtcgacattcctgagattcctgggttc
aaggacttggagcccctggagcagttcatcgcacaggtcgatctgtgtgtggactgcacaactggctgcctcaaagggctt
gccaacgtgcagtgttctgacctgctcaagaagtggctgccgcaacgctgtgcgacctttgccagcaagatccagggccag
gtggacaagatcaagggggccggtggtgactaagcggccgctcgagcatgcatctagaaataattcttactgt
Primer
(DNA)
(SEQ ID NO: 802)
acaaaattcaaaattttatcgatactagttggcctaactggccggtaccaag
Primer
(DNA)
(SEQ ID NO: 803)
atccgatttaaattcgaattcgctagcttatcacttatcgtcgtcatcc
NFAT consensus sequence:
(SEQ ID NO: 804)
(A/T)GGAAA(A/N)(A/T/C)N
Current NFAT RE (Form System Biosciences. The sequence is from the mouse IL2
promoter
(DNA)
(SEQ ID NO: 805)
aagaggaaaatttgtttcatacagaaggcgtt
Mouse IL2 Promoter (highlighted in green the NFAT RE used, highlighted in
yellow is the start codon)
(DNA)
(SEQ ID NO: 806)
aactagagacatataaaataacaccaacatccttagatacaacccttcctgagaatttattggacatcatactcttttta
aaaagcataataaacatcaagacacttacacaaaatatgttaaattaaatttaaaacaacaacgacaaaatagtacctca
agctcaacaagcattttaggtgtccttagcttactatttctctggctaactgtatgaagccatctatcaccctgtgtgca
attagctcattgtgtagataagaaggtaaaaccatcttgaaacaggaaaccaatatccttcctgtctaatcaacaaatct
aaaagatttattcttttcatctatctcctcttgcgtttgtccaccacaacaggctgcttacaggttcaggatggttttga
caaagagaacattttcatgagttacttttgtgtctccaccccaaagaggaaaatttgtttcatacagaaggcgttcattg
tatgaattaaaactgccacctaagtgtgggctaacccgaccaagagggatttcacctaaatccattcagtcagtgtatgg
gggtttaaagaaattccagagagtcatcagaagaggaaaaacaaaaggtaatgctttctgccacacaggtagactctttg
aaaatatgtgtaatatgtaaaacatcgtgacacccccatattatttttccagcattaacagtataaattgcctcccatgc
tgaagagctgcctatcacccttgctaatcactcctcacagtgacctcaagtcctgcaggcatgtacagcatgcagctcgc
atcctgtgtcac
NFAT RE (Form PRomega. The sequence is from the humane IL2 promoter
(DNA)
(SEQ ID NO: 807)
ggaggaaaaactgtttcatacagaaggcgt
Possible NFAT RE from ET-1 promoter
(DNA)
(SEQ ID NO: 808)
tccagggaaaatcggagtagaacaagagggatg
Possible NFAT RE from ET-1 promoter
(DNA)
(SEQ ID NO: 809)
actgttggaaaacgtaaacacgttattaaacggt
Possible NFAT RE from human CD3γ
(DNA)
(SEQ ID NO: 810)
tccttaacggaaaaacaaaa
Possible NFAT RE from human CD3γ
(DNA)
(SEQ ID NO: 811)
aaaggaaaaagtatatgttc
Possible NFAT RE from human IL3 promoter
(DNA)
(SEQ ID NO: 812)
atgccatggaaagggtg
Possible NFAT RE from human GPC6
(DNA)
(SEQ ID NO: 813)
aaggggaaatgttgagtctaga
Possible NFAT RE from human growth hormone-releasing hormone
(DNA)
(SEQ ID NO: 814)
AACTTGGAAAAGCATAG
NFATc1 promoter large
(DNA)
(SEQ ID NO: 815)
ttatgccgtctagaggagacatactttctactcaaagctacacacatagactacaacgatgggaaaagacgacacaccaa
cagcgacttcaggaaagctggagtggctgctaatgttagacaaaataggctttttaaaaaaggttttattaaagaggaat
gtttcgtaatgataaaagcactaatctgtgagaaagatacaacaatgataaacatacgtgcagctaataagagagctcca
aaatctatgaagcaaaaactcacagaatgaggggagaagcagttctacaacagagaatggggacttcgatactccacttt
caataatggatacaacaaccaggcagataacaaggcaacagaaggcctgaacaacagtataaaccaattagacctaccag
atatctatagctagcacactccacccaacgacagcagaatacacattcttctcaagcgcacaagtaacatcctccaggat
gggccatgttctaggccatcaaacaaactcaggtggtttgaggccagaggcctctcttttaaccaccacactagggcctt
cggaggaggcaagcagagagttgtcaaagaggccctcaggactgggtgcagtggctcatgactgtaatcccagcacttta
gaaggctgaggcacaaggatcttttgagctcaggagttcaagaaatgagcacttatccactgggcgcggtggctcacgcc
agtaatccagcactttgggaggcttaggcgggcggatcaagaggtcagaagctcaagaccagcctgaccaacatggtgaa
accccgtctctactaaaagtacaaaaattagccgggcgtggtggcgcacacctgtaatcccagctacttgggaggctgag
gcaggagaatcacttgaacccgggaggtggaggttgcagtgagtggagatcacaccattgcaccccagcctgggcaacag
agcgagactccgtctcaaaaaaaaaaaaaaaaaaaaagaaagaaagaaaaagaaaaaaaaagtgagcatgtattttgcca
gagtctggagattagaattaaattagcaaaccagaattatagaaaaagctatttacttttaagtaaacagctgagatttt
tttttttaagtcagtgtgaatgaagctcacagccatggttggagctgagaaagaaggatttccctttagttatgcacctg
tgtcagcaccttctgactttccttctaaagtctggggtgttcctgaggatccgtaagtttggggttcagggtttctacag
catgctgttacttgtgaaacatctctttaaccatgtcccagagttgcccaggagtttaagaccagcctgagcaacatagc
aagacctcatctcaacaacaacaaaaattagaaataaattagccaggtgtggtgacatgtgcctgtagtcccagctactc
agaaggctgaggcatgaggatcacttgggcccaggaagttggggctgcagggagccctgttcatgccgctgcactccagc
ctgcaagacagagcagaaaaaaagaatcaggatcctgggcagagggaggagaggggaccggggtccagcaagcacttggg
gattgactgaatggcgttggggagagatgactccaaagtcctggagtgggtgagaatgactgcgagtggcttttaggtgg
ggaggttcctgcctggccactccgggaggggacgtggggctgaagggtatcaggtgccgtgctgagcagtttggccttga
tcctaatgccctggacacacgtctagggtaggaaagttgactgatccattggtgatctgagtttttagacatggtggtag
tccatgaggtgggtgttcatgctaagagtttagacagggaaacctatgaagcccttagcaaccctccagggaaggggcgt
ggttaaagagatgtttcataagtaacagcatggtatagaaactctgaaccccaaatgtatgggtcctcaggaacacccca
gactttagaaggaaagtcagaaggtgctgacacgggtgtataactaaagggaaatccttctttctcagctccaaccatgg
ctgtgaggttcattcacactgacttaaaaaaaaaaatctcagtttacttaaaagtaaatagctttttctataattctggt
ttgctaatttaatcctagtctccagaccctggctaaataaatgcccatttctccagatggtctcaagagtctctggacat
cgtgggggcccttccctgttggttggaaggtgcctcaggaagaagggggtggattctgagttgagtcaaaacctcaaaga
cccctgatgggaaaagctctcaagtgaccaccgctgtgggccagaatgcaaaactgcaggaacagaacattcgcaggaac
agaacacagtcgtattaagtgattttcccgagcaggaagtggcatctggcctgcggttcagtagggggaggaaagggtgg
gcgcacctgcccctggctggcgcacctgccaggtagccccacgcggcaccgcgtgtgccgagcgcccctgaggatggaaa
gccccacgcggggcaggtggcacccaccctccgaagacgggacgggatggagcgttgagcttcggggcagctccggcccg
gcccgcgctggagacgcccgcatctgccaggatggcgtctcatagccctggtgctcacacatgacgccaggaagccccag
caacagtgaccgcccaggctctagaaaatattggacggggtggatgaacacccaagtgcgctccaggagaagggatttgg
caccccaaggggcttttaaaacggtaagcttctaggggtgtctttgcccccaataatccatagaaacaacagtcatctaa
aaatagtcttgttttctgtcctaagctccttttaactttgttagtcatcaccaatcctaaaataaaacccgtgtaacgtc
tcccctagtagcggctataaacaaacctacgaggaggcaggaggaagaggaaaggggcgcagggcgctcggggagcagag
ccgggggcccgcggtggccgcagaggccgggccggggcgcagaggccgggcgagctggccgcgctctgggccgccgcctc
cggaactccctgcgcctggcgcgcggccaccgtggtcccggcaacggcattaaacagagggaaacagacccgggattccg
tcacccgggcggggggataaggacggctttgagagcagacaggaaaagggagcttttctgcatggggtgaaaaaattatt
tattgaaggaggaggaggcggcagcggaggaaggggaggggcgggaggaggaggaagagccggccgcccccgccccggcc
ccggctcctcaggagccaagggcagcctcgccaggtcggtcccgggctcgaggaccgcggctggggtcgaggggctcagt
ctcccacgtgaccggctgggcgcgccccgccagacccggcctcgggattccctcctcccggcgagtctccgcccgccccg
tcctggaggtggggagaaggagggcggggcgggggggacggaaactctccccgccaaatcctggccccaggcctggggac
actcgcggcgggaagatttggaggggaggggagggggaggggcgtgggggcgcggcctcgctggagtccccctgaccccc
cgacccccgcccaccggcctgggcgtcctcccgcggcccctcctcccctcccggcgcccggtgctctggggcgcgtgcca
cgcctggctcggcgccgtaggggcccccgcaggtagagacccctggaaatggcctcgacgccgcaggagcgaggcggcca
ccaccccgctaatccgggcacgtctctccaggccgaggcctgcggtggaaaagccggggttccatttgtgctgagtcggg
gcggccgaatggagccaggcctcgggacgcgggacggacgggctctggccgcgcaccttcgcgggctctgcagcgcccga
ccgcctcccccggcagggaggaggcgcttgtggggggcacccacggggcacagtgatccctgggggtctgcggacctcct
gggccccgcagcagacacgagtttagcctttgggtttagtttaaatcacataagggtgtcgtgcaatcgatttatggttt
ctacacaccagacactttaacctccaaccccccccatccaaagccaacaagaaaatgcggtgccgtgttggcagctgagc
tgcgcccgaagagacgcagggagacgtaagagaggaaagtgtgagtggccggggggcctccccccgtcagaagtcgcgca
gtcgcgcccataaaacgccccctccgggcggctagggcaggtgagcgcgtccccgggcctccccacgccggcccctgcca
caggccgtctaggtcgagcagatatttacagaataaaaatgacaataactcgacgtcccgggacggccacgcaatctgtt
agtaatttagcgggatgggaatttcctttctagggcctgccagtgaagcgcttttccaaatttccacagcgggggaagcc
tgcgattttacataatgacttcagcatgccgggctttctcgacacccctccccggcccccggcccccgccccccgcccct
tttccagcagggccgggctccctccggacacccgcgtggactcaggcgtcccgtctggcccgttcgcccccgtttccccc
gccagccccagcgcccccctgcccggcccccggattccccgttcccgcccctacgcccccatcccctccccgtgcgcccc
tccccgtgcgcccccctccccgtgcgccccccctccccgtgcgcccccctccccgtgcgccccccctccccgggcgcccc
cctccccgggcgccccccctccccgtgcgcccccccctccccgtgcgccccccctccccgtgcgcgccccgcctcttgcg
cccctgcccccaggcgagcggctgccgcggcgcggggaggggcgggcgctcggcgactcgtccccggggccccgcgcggg
cccgggcagcaggggcgtgatgtcacggcagggagggggcgcgggagccgccgggccggcggggaggcgggggaggtgtt
ttccagctttaaaaaggcaggaggcagagcgcggccctgcgtcagagcgagactcagaggctccgaactcgccggcggag
tcgccgcgccagatcccagcagcagggcgcgggcaccggggcgcgggcagggctcggagccaccgcgcaggtcctagggc
cgcggccgggccccgccacgcgcgcacacgcccctcgatg
NFATc3 promoter sequence
(DNA)
(SEQ ID NO: 816)
gcagccaggcagggtgggcgcgcgtagggggcggggccgggcgcgcggcagggcgcgagagcgcacccgcggcggcggtg
gcggcgactgtgggggggcggcggggaacattggctaagccgacagtggaggcttaggcaccggtggcgggcggctgcgg
ttcctggtgctgctcggcgcgcggccagctttcggaacggaacgctcggcgtcgcgggccccgcccggaaagtttgccgt
ggagtcgcgacctcttggcccgcgcggcccggcatgaagcggcgttgaggagctgctgccgccgcttgccgctgccgccg
ccgccgcctgaggaggagctgcagcaccctgggccacgccg
NFATc2 promoter sequence 1
(DNA)
(SEQ ID NO: 817)
cagagagaggctgcgttcagactggggcactgccatcccctccgcatca
tggggtctgtggaccaaggtaactgactctcgatcccttccagccttttccgctcgctcctcccggccctttcctgctgc
tcccgtcccgggcagcactttcagctcccggcagaggtcggtgcgggaggcctggggaccccgctcgccctcggcgcaca
ggtagcggggcccgcggaggggcgcccgcgccccggccagggaagggacacttgggaaggcgactttggacaactttacg
cgggggcagggaagtgtcccaggccgggattccctaggccagtctgtcgggaggattttcctctccacgggacaccggga
gggattctcgctactaaccgctggctgtttaaccgtttcagcactcggcttttgacagcaa
NFATc2 promoter sequence 2
(DNA)
(SEQ ID NO: 818)
catcatggggtctgtggaccaaggtaactgactctcgatcccttccagccttttccgctcgctc
NFATc1 response element consensus
(DNA)
(SEQ ID NO: 819)
cattttttccat
NFATc1 response element consensus
(DNA)
(SEQ ID NO: 820)
tttttcca
NFAT response elements contained within the Foxp3 enhancer region
(DNA)
(SEQ ID NO: 821)
acttgaaaatgagataaatgttcacctatgttggcttctagtctcttttatggcttcatt
ttttccatttactatagaggttaagagtgtgggtactggagccagactgtctgggacaa
muE6 IgD/CD8/41BB/CD3z
(DNA)
(SEQ ID NO: 822)
atggccctgcccgtgaccgctttgctgctccccctggcgctgctgctgcacgccgccaggccagaggtgaaggtggtggag
tctgggggagacttagtgaagcctggagggtccctgaaactctcctgtgtagtctctggattcactttcagtagatatggc
atgtcttgggttcgccagactccaggcaagaggctggagtgggtcgcaaccattagtggtggcggtacttacatctactat
ccagacagtgtgaaggggcgattcaccatctccagagacaatgccaagaacaccctgtacctgcaaatgagcagtctgaag
tctgaggacacagccatgtatcactgtacaagggataactacggtaggaactacgactacggtatggactactggggtcaa
ggaacctcagtcaccgtctcctcaggcggtggcggatccggcggtggcggatccggcggtggcggatcccaaattgttctc
acccagtctccagcaatcatgtctgcatctccaggggaggaggtcaccctaacctgcagtgccacctcaagtgtaagttac
atacactggttccagcagaggccaggcacttctcccaaactctggatttatagcacatccaacctggcttctggagtccct
gttcgcttcagtggcagtggatatgggacctcttactctctcacaatcagccgaatggaggctgaagatgctgccacttat
tactgccagcaaaggagtagttccccattcacgttcggctcggggacaaagttggaaataaaagagtctccaaaggcaca
ggcctcctcagtgcccactgcacaaccccaagcagagggcagcctcgccaaggcaaccacagccccagccaccacccgta
acacaggaagaggcggcgaagagaagaaaaaggagaaggagaaagaggaacaagaagagagagagacaaagacaccaatc
tacatttgggccccgctcgcaggcacatgtggagtgctcctcctctccctggtgattaccctgtactgcaaaaggggccgc
aaaaaactcctttacatttttaagcagccttttatgaggccagtacagacgactcaagaggaagacgggtgctcatgccgc
tttcctgaggaggaggaaggagggtgcgaactgcgcgttaagttctcccgatcagccgacgcgcctgcttacaagcagggc
cagaaccaactgtacaacgagctgaatctcggtagacgggaagagtacgacgtgttggacaaacggagaggccgcgaccca
gaaatgggcggcaagcctcgcaggaaaaacccccaggagggactgtacaatgagttgcagaaagataagatggcagaagct
tatagcgagatcggaatgaagggggaaaggagacgagggaaaggacacgacggcctttatcagggcctgtccacagcaaca
aaagatacgtatgacgccctccatatgcaggcacttccaccacggtgataa
(amino acids)
(SEQ ID NO: 823)
MALPVTALLLPLALLLHAARPEVKVVESGGDLVKPGGSLKLSCVVSGFTFSRYGMSWVRQTPGKRLEWVATISGGGTYIY
YPDSVKGRFTISRDNAKNTLYLQMSSLKSEDTAMYHCTRDNYGRNYDYGMDYWGQGTSVTVSSGGGGSGGGGSGGGGSQI
VLTQSPAIMSASPGEEVTLTCSATSSVSYIHWFQQRPGTSPKLWIYSTSNLASGVPVRFSGSGYGTSYSLTISRMEAEDA
ATYYCQQRSSSPFTFGSGTKLEIKESPKAQASSVPTAQPQAEGSLAKATTAPATTRNTGRGGEEKKKEKEKEEQEERETK
TPIYIWAPLAGTCGVLLLSLVITLYCKRGRKKLLYIFKQPFMRPVQTTQEEDGCSCRFPEEEEGGCELRVKFSRSADAPA
YKQGQNQLYNELNLGRREEYDVLDKRRGRDPEMGGKPRRKNPQEGLYNELQKDKMAEAYSEIGMKGERRRGKGHDGLYQG
LSTATKDTYDALHMQALPPR**
muC2 IgD/CD8/41BB/CD3z
(DNA)
(SEQ ID NO: 824)
atggccctgcccgtgaccgctttgctgctccccctggcgctgctgctgcacgccgccaggccagaggtccagctggaggag
tcagggggaggcttagtgaagcctggagggtccctgaaactctcctgtgcagcctctggattcactttcagtggctatgcc
atgtcttgggttcgccagactccggagaagaggctggagtgggtcgcaaccattagtagtggtggtacttatatctactat
ccagacagtgtgaaggggcgattcaccatctccagagacaatgccaagaacaccctgtacctgcaaatgagcagtctgagg
tctgaggacacggccatgtattactgtgcaagacttgggggggataattactacgaatacttcgatgtctggggcgcaggg
accacggtcaccgtctcctccggcggtggcggatccggcggtggcggatccggcggtggcggatccgacattgtgatcaca
cagtctacagcttccttaggtgtatctctggggcagagggccaccatctcatgcagggccagcaaaagtgtcagtacatct
ggctatagttatatgcactggtaccaacagagaccaggacagccacccaaactcctcatctatcttgcatccaacctagaa
tctggggtccctgccaggttcagtggcagtgggtctgggacagacttcaccctcaacatccatcctgtggaggaggaggat
gctgcaacctattactgtcagcacagtagggagcttccgttcacgttcggaggggggaccaagctggagataaaagagtc
tccaaaggcacaggcctcctcagtgcccactgcacaaccccaagcagagggcagcctcgccaaggcaaccacagccccag
ccaccacccgtaacacaggaagaggcggcgaagagaagaaaaaggagaaggagaaagaggaacaagaagagagagagaca
aagacaccaatctacatttgggccccgctcgcaggcacatgtggagtgctcctcctctccctggtgattaccctgtactg
caaaaggggccgcaaaaaactcctttacatttttaagcagccttttatgaggccagtacagacgactcaagaggaagacgg
gtgctcatgccgctttcctgaggaggaggaaggagggtgcgaactgcgcgttaagttctcccgatcagccgacgcgcctgc
ttacaagcagggccagaaccaactgtacaacgagctgaatctcggtagacgggaagagtacgacgtgttggacaaacggag
aggccgcgacccagaaatgggcggcaagcctcgcaggaaaaacccccaggagggactgtacaatgagttgcagaaagataa
gatggcagaagcttatagcgagatcggaatgaagggggaaaggagacgagggaaaggacacgacggcctttatcagggcct
gtccacagcaacaaaagatacgtatgacgccctccatatgcaggcacttccaccacggtgataa
(amino acids)
(SEQ ID NO: 825)
MALPVTALLLPLALLLHAARPEVQLEESGGGLVKPGGSLKLSCAASGFTFSGYAMSWVRQTPEKRLEWVATISSGGTYIY
YPDSVKGRFTISRDNAKNTLYLQMSSLRSEDTAMYYCARLGGDNYYEYFDVWGAGTTVTVSSGGGGSGGGGSGGGGSDIV
ITQSTASLGVSLGQRATISCRASKSVSTSGYSYMHWYQQRPGQPPKLLIYLASNLESGVPARFSGSGSGTDFTLNIHPVE
EEDAATYYCQHSRELPFTFGGGTKLEIKESPKAQASSVPTAQPQAEGSLAKATTAPATTRNTGRGGEEKKKEKEKEEQEE
RETKTPIYIWAPLAGTCGVLLLSLVITLYCKRGRKKLLYIFKQPFMRPVQTTQEEDGCSCRFPEEEEGGCELRVKFSRSA
DAPAYKQGQNQLYNELNLGRREEYDVLDKRRGRDPEMGGKPRRKNPQEGLYNELQKDKMAEAYSEIGMKGERRRGKGHDG
LYQGLSTATKDTYDALHMQALPPR**
muE6 CD28/CD28/CD28/CD3z
(DNA)
(SEQ ID NO: 826)
atggccctgcccgtgaccgctttgctgctccccctggcgctgctgctgcacgccgccaggccagaggtgaaggtggtgga
gtctgggggagacttagtgaagcctggagggtccctgaaactctcctgtgtagtctctggattcactttcagtagatatg
gcatgtcttgggttcgccagactccaggcaagaggctggagtgggtcgcaaccattagtggtggcggtacttacatctac
tatccagacagtgtgaaggggcgattcaccatctccagagacaatgccaagaacaccctgtacctgcaaatgagcagtct
gaagtctgaggacacagccatgtatcactgtacaagggataactacggtaggaactacgactacggtatggactactggg
gtcaaggaacctcagtcaccgtctcctcaggcggtggcggatccggcggtggcggatccggcggtggcggatcccaaatt
gttctcacccagtctccagcaatcatgtctgcatctccaggggaggaggtcaccctaacctgcagtgccacctcaagtgt
aagttacatacactggttccagcagaggccaggcacttctcccaaactctggatttatagcacatccaacctggcttctg
gagtccctgttcgcttcagtggcagtggatatgggacctcttactctctcacaatcagccgaatggaggctgaagatgct
gccacttattactgccagcaaaggagtagttccccattcacgttcggctcggggacaaagttggaaataaaaaaacacct
ttgtccaagtcccctatttcccggaccttctaagcccttttgggtgctggtggtggttggtggagtcctggcttgctata
gcttgctagtaacagtggcctttattattttctgggtgagaagcaagcggtctcggctcctgcattctgattacatgaac
atgaccccaagaagaccaggccccaccaggaaacattaccagccctacgctccgccacgcgacttcgctgcctaccggtc
ccgcgttaagttctcccgatcagccgacgcgcctgcttacaagcagggccagaaccaactgtacaacgagctgaatctcg
gtagacgggaagagtacgacgtgttggacaaacggagaggccgcgacccagaaatgggcggcaagcctcgcaggaaaaac
ccccaggagggactgtacaatgagttgcagaaagataagatggcagaagcttatagcgagatcggaatgaagggggaaag
gagacgagggaaaggacacgacggcctttatcagggcctgtccacagcaacaaaagatacgtatgacgccctccatatgc
aggcacttccaccacggtgataa
(amino acids)
(SEQ ID NO: 827)
MALPVTALLLPLALLLHAARPEVKVVESGGDLVKPGGSLKLSCVVSGFTFSRYGMSWVRQTPGKRLEWVATISGGGTYIY
YPDSVKGRFTISRDNAKNTLYLQMSSLKSEDTAMYHCTRDNYGRNYDYGMDYWGQGTSVTVSSGGGGSGGGGSGGGGSQI
VLTQSPAIMSASPGEEVTLTCSATSSVSYIHWFQQRPGTSPKLWIYSTSNLASGVPVRFSGSGYGTSYSLTISRMEAEDA
ATYYCQQRSSSPFTFGSGTKLEIKKHLCPSPLFPGPSKPFWVLVVVGGVLACYSLLVTVAFIIFWVRSKRSRLLHSDYMN
MTPRRPGPTRKHYQPYAPPRDFAAYRSRVKFSRSADAPAYKQGQNQLYNELNLGRREEYDVLDKRRGRDPEMGGKPRRKN
PQEGLYNELQKDKMAEAYSEIGMKGERRRGKGHDGLYQGLSTATKDTYDALHMQALPPR**
muC2 CD28/CD28/CD28/CD3z
(DNA)
(SEQ ID NO: 828)
atggccctgcccgtgaccgctttgctgctccccctggcgctgctgctgcacgccgccaggccagaggtccagctggaggag
tcagggggaggcttagtgaagcctggagggtccctgaaactctcctgtgcagcctctggattcactttcagtggctatgcc
atgtcttgggttcgccagactccggagaagaggctggagtgggtcgcaaccattagtagtggtggtacttatatctactat
ccagacagtgtgaaggggcgattcaccatctccagagacaatgccaagaacaccctgtacctgcaaatgagcagtctgagg
tctgaggacacggccatgtattactgtgcaagacttgggggggataattactacgaatacttcgatgtctggggcgcaggg
accacggtcaccgtctcctccggcggtggcggatccggcggtggcggatccggcggtggcggatccgacattgtgatcaca
cagtctacagcttccttaggtgtatctctggggcagagggccaccatctcatgcagggccagcaaaagtgtcagtacatct
ggctatagttatatgcactggtaccaacagagaccaggacagccacccaaactcctcatctatcttgcatccaacctagaa
tctggggtccctgccaggttcagtggcagtgggtctgggacagacttcaccctcaacatccatcctgtggaggaggaggat
gctgcaacctattactgtcagcacagtagggagcttccgttcacgttcggaggggggaccaagctggagataaaaaaacac
ctttgtccaagtcccctatttcccggaccttctaagcccttttgggtgctggtggtggttggtggagtcctggcttgctat
agcttgctagtaacagtggcctttattattttctgggtgagaagcaagcggtctcggctcctgcattctgattacatgaac
atgaccccaagaagaccaggccccaccaggaaacattaccagccctacgctccgccacgcgacttcgctgcctaccggtcc
cgcgttaagttctcccgatcagccgacgcgcctgcttacaagcagggccagaaccaactgtacaacgagctgaatctcggt
agacgggaagagtacgacgtgttggacaaacggagaggccgcgacccagaaatgggcggcaagcctcgcaggaaaaacccc
caggagggactgtacaatgagttgcagaaagataagatggcagaagcttatagcgagatcggaatgaagggggaaaggaga
cgagggaaaggacacgacggcctttatcagggcctgtccacagcaacaaaagatacgtatgacgccctccatatgcaggca
cttccaccacggtgataa
(amino acids)
(SEQ ID NO: 829)
MALPVTALLLPLALLLHAARPEVQLEESGGGLVKPGGSLKLSCAASGFTFSGYAMSWVRQTPEKRLEWVATISSGGTYIY
YPDSVKGRFTISRDNAKNTLYLQMSSLRSEDTAMYYCARLGGDNYYEYFDVWGAGTTVTVSSGGGGSGGGGSGGGGSDIV
ITQSTASLGVSLGQRATISCRASKSVSTSGYSYMHWYQQRPGQPPKLLIYLASNLESGVPARFSGSGSGTDFTLNIHPVE
EEDAATYYCQHSRELPFTFGGGTKLEIKKHLCPSPLFPGPSKPFWVLVVVGGVLACYSLLVTVAFIIFWVRSKRSRLLHS
DYMNMTPRRPGPTRKHYQPYAPPRDFAAYRSRVKFSRSADAPAYKQGQNQLYNELNLGRREEYDVLDKRRGRDPEMGGKP
RRKNPQEGLYNELQKDKMAEAYSEIGMKGERRRGKGHDGLYQGLSTATKDTYDALHMQALPPR**
CD19 IgD/CD8/41BB/CD3z
(DNA)
(SEQ ID NO: 830)
atggccctgcccgtgaccgctttgctgctccccctggcgctgctgctgcacgccgccaggccagacatacagatgacgca
gacgaccagcagcctttccgcttccctgggcgaccgagtaaccattagttgtagagcatctcaggatatttctaagtatc
tgaattggtaccaacagaaacctgatggcactgtcaagctcttgatatatcacaccagtcgactccattcaggcgtccct
tccagattcagtgggagtggcagcgggactgattactccctcactatctctaacctggaacaggaagacatcgctacata
cttctgtcagcagggaaacactctcccctatacctttgggggaggaaccaagttggaaataacaggcggtggcggatccg
gcggtggcggatccggcggtggcggatccgaggtgaaactgcaggagtcaggacctggcctggtggcgccctcacagagc
ctgtccgtcacatgcactgtctcaggggtctcattacccgactatggtgtaagctggattcgccagcctccacgaaaggg
tctggagtggctgggagtaatatggggtagtgaaaccacatactataattcagctctcaaatccagactgaccatcatca
aggacaactccaagagccaagttttcttaaaaatgaacagtctgcaaactgatgacacagccatttactactgtgccaaa
cattattactacggtggtagctatgctatggactactggggccaaggaacctcagtcaccgtctcctcagagtctccaaa
ggcacaggcctcctcagtgcccactgcacaaccccaagcagagggcagcctcgccaaggcaaccacagccccagccacca
cccgtaacacaggaagaggcggcgaagagaagaaaaaggagaaggagaaagaggaacaagaagagagagagacaaagaca
ccaatctacatttgggccccgctcgcaggcacatgtggagtgctcctcctctccctggtgattaccctgtactgcaaaag
gggccgcaaaaaactcctttacatttttaagcagccttttatgaggccagtacagacgactcaagaggaagacgggtgctc
atgccgctttcctgaggaggaggaaggagggtgcgaactgcgcgttaagttctcccgatcagccgacgcgcctgcttacaa
gcagggccagaaccaactgtacaacgagctgaatctcggtagacgggaagagtacgacgtgttggacaaacggagaggccg
cgacccagaaatgggcggcaagcctcgcaggaaaaacccccaggagggactgtacaatgagttgcagaaagataagatggc
agaagcttatagcgagatcggaatgaagggggaaaggagacgagggaaaggacacgacggcctttatcagggcctgtccac
agcaacaaaagatacgtatgacgccctccatatgcaggcacttccaccacggtgataa
(amino acids)
(SEQ ID NO: 831)
MALPVTALLLPLALLLHAARPDIQMTQTTSSLSASLGDRVTISCRASQDISKYLNWYQQKPDGTVKLLIYHTSRLHSGVP
SRFSGSGSGTDYSLTISNLEQEDIATYFCQQGNTLPYTFGGGTKLEITGGGGSGGGGSGGGGSEVKLQESGPGLVAPSQS
LSVTCTVSGVSLPDYGVSWIRQPPRKGLEWLGVIWGSETTYYNSALKSRLTIIKDNSKSQVFLKMNSLQTDDTAIYYCAK
HYYYGGSYAMDYWGQGTSVTVSSESPKAQASSVPTAQPQAEGSLAKATTAPATTRNTGRGGEEKKKEKEKEEQEERETKT
PIYIWAPLAGTCGVLLLSLVITLYCKRGRKKLLYIFKQPFMRPVQTTQEEDGCSCRFPEEEEGGCELRVKFSRSADAPAY
KQGQNQLYNELNLGRREEYDVLDKRRGRDPEMGGKPRRKNPQEGLYNELQKDKMAEAYSEIGMKGERRRGKGHDGLYQGL
STATKDTYDALHMQALPPR**

Those skilled in the art will recognize, or be able to ascertain using no more than routine experimentation, many equivalents to the specific embodiments of the invention specifically described herein.

1.-97. (canceled)

98. An immune cell transfected or transduced with a cleavage enzyme for the treatment of cancer.

99. The immune cell of claim 98, wherein the cancer is a MUC1 positive cancer.

100. The immune cell of claim 98, wherein the immune cell is a T cell or NK cell.

101. The immune cell of claim 100, wherein the cell is derived from the patient to be treated.

102. The immune cell of claim 98, wherein the cleavage enzyme is an MMP or ADAM family member or a catalytically active fragment thereof.

103. The immune cell of claim 102, wherein the cleavage enzyme is MMP2, MMP9, MMP3, MMP14, ADAM17, ADAM28, or ADAM TS16.

104. An immune cell transfected or transduced with both a CAR comprising an antibody fragment, and a cleavage enzyme or catalytically active fragment thereof for the treatment of cancer.

105. The immune cell of claim 104, wherein the cancer is a MUC1 positive cancer.

106. The immune cell of claim 104, wherein the immune cell is a T cell or NK cell.

107. The immune cell of claim 104, wherein the antibody fragment of the CAR on the T cell directs the cell to a MUC1* positive tumor.

108. The immune cell of claim 104, wherein the antibody fragment of the CAR transduced into the immune cell recognizes a form of MUC1 after it is cleaved by the cleavage enzyme that is also transduced into the immune cell.

109. The immune cell of claim 108, wherein the antibody fragment of the CAR is derived from MNC2 or MNE6 and the cleavage enzyme is MMP9 or fragment of MMP9 or an activated form of MMP9.

110. The immune cell of claim 104, which is further transfected or transduced with an activator of the cleavage enzyme.

111. The immune cell of claim 110, wherein the cleavage enzyme is MMP9, and the activator of the cleavage enzyme is MMP3.

112. The immune cell of claim 104, wherein nucleic acid encoding the cleavage enzyme is linked to an inducible promoter.

113. The immune cell of claim 112, wherein expression of the cleavage enzyme is induced by an event that occurs specifically when the immune cell mounts an immune response to a target tumor cell.

114. The immune cell of claim 112, wherein the inducible promoter is induced by the presence of a NFAT family protein so as to express the cleavage enzyme, or the inducible promoter is a promoter for expression of NFAT protein.

115. The immune cell of claim 114, wherein the NFAT protein is NFATc1 also known as NFAT 2.

116. The immune cell of claim 112, wherein the immune cell is a T cell or NK cell.

117. The immune cell of claim 104, wherein the antibody fragment recognizes a form of MUC1 or MUC1* that is created when the cleavage enzyme cleaves MUC1 or MUC1*.

118. The immune cell of claim 112, wherein the antibody fragment is part of a CAR.

119. The immune cell of claim 112, wherein expression of the cleavage enzyme on the inducible promoter is induced when the antibody fragment of the CAR engages or binds to a MUC1 or MUC1* on the tumor.

120. (canceled)

121. A nucleic acid construct comprising nucleic acid encoding a cleavage enzyme or catalytically active fragment thereof located downstream of an NFAT promoter.

122. The nucleic acid construct of claim 121, wherein the NFAT is NFATc1.

123. The nucleic acid construct of claim 121, wherein the sequence of the promoter is any of the promoter sequence set forth as SEQ ID NOS:781-783, or a fragment or mutation thereof, which retains the activity of inducing expression of the cleavage enzyme or catalytically active fragment thereof.

124. The nucleic acid construct of claim 121, wherein the cleavage enzyme is MMP9.

125. The nucleic acid construct of claim 121, which is a plasmid.

126. An immune cell transfected or transduced with the nucleic acid construct of claim 121.

127. The immune cell according to claim 126, which is T cell or NK cell.

128. A method of treating or preventing cancer comprising administering to a patient the cell of claim 126.

129. The nucleic acid construct comprising nucleic acid encoding a cleavage enzyme or catalytically active fragment thereof located downstream of at least one NFAT response element.

130. The nucleic acid construct according to claim 129, wherein the NFAT response element comprises at least 2, 3, 4 response elements.

131. The nucleic acid construct of claim 129, wherein the sequence of the response element is any of the sequence set forth as SEQ ID NOS: 804-816, or a fragment or mutation thereof, which retains the activity of inducing expression of the cleavage enzyme or catalytically active fragment thereof.

132. The nucleic acid construct of claim 129, wherein the cleavage enzyme is MMP9.

133. The nucleic acid construct of claim 129, which is a plasmid.

134. An immune cell transfected or transduced with the nucleic acid construct of claim 129.

135. The immune cell according to claim 134, which is T cell or NK cell.

136. A method of treating or preventing cancer comprising administering to a patient the cell of claim 134.

137. A method of pre-activating an immune cell transduced or transfected with nucleic acid encoding CAR that is specific for MUC1* and/or MUC1 specific cleavage enzyme by co-culturing the immune cell in vitro on a surface that presents a peptide having the sequence of truncated extracellular domain of MUC1, thus obtaining pre-activated immune cell.

138. The method of claim 137, wherein the immune cell is T cell.

139. The method of claim 137, comprising further administering to a patient a composition comprising the obtained pre-activated immune cell.

140. The method of claim 137, wherein the surface is a bead, a cell culture plate or a cell.

141. The method of claim 140, wherein the cell is a MUC1* expressing cell

142. The method of claim 141, wherein the cell is a MUC1* expressing cancer cell.

143. The method of claim 142, wherein the cell is derived from the patient.

144. The method of claim 137, comprising removing the surface before administering the composition to the patient.

145. The method of claim 144, wherein when the surface is a cell, the cell is treated with ultraviolet light or is chemically inactivated such that the cell replicates a few times, and then administering to the patient.

146. An immune cell transfected or transduced with a plasmid encoding a CAR and a plasmid encoding a non-CAR species that is expressed from an inducible promoter.

147. The immune cell of claim 146 wherein the CAR comprises an antibody fragment, scFv or a peptide that binds to a tumor antigen.

148. The immune cell of claim 147, wherein tumor antigen is MUC1*.

149. The immune cell of claim 147, wherein the antibody fragment is derived from non-human, human or humanized MNC2, MNE6, MNC3 or MNC8.

150. The immune cell of claim 147, wherein the antibody fragment, scFv or peptide binds to a surface antigen of a B cell or a B cell precursor, CD19, CD20, CD22, BCMA, CD30, CD138, CD123, CD33 or LeY antigen.

151. The immune cell of claim 146, wherein the non-CAR species is expressed from an inducible promoter that is activated by elements of an activated immune cell.

152. The immune cell of claim 146, wherein the non-CAR species is expressed from an NFAT inducible promoter.

153. The immune cell of claim 152, wherein the NFAT is NFATc1, NFATc3 or NFATc2.

154. The immune cell of claim 146, wherein the non-CAR species is a cleavage enzyme.

155. The immune cell of claim 146, wherein the cleavage enzyme is MMP2, MMP3, MMP9, MMP13, MMP14, MMP16, ADAM10, ADAM17, or ADAM28, or a catalytically active fragment thereof.

156. The immune cell of claim 146, wherein the non-CAR species is a cytokine.

157. The immune cell of claim 156, wherein the cytokine is IL-7, IL-15 or IL-7 and IL-15.

158. A method of treating or preventing cancer comprising administering to a patient thereof, the immune cell of claim 146.
